SEQUENCE LISTING (258 sequences)

SEQ ID NO: 1 - DNA, 25 bp, Homo sapiens
  source 1..25: mol_type = other DNA
gctagggtggtcttttagaaatgca

SEQ ID NO: 2 - DNA, 25 bp, Homo sapiens
  source 1..25: mol_type = other DNA
ggtcaagctagggtggtcttttaga

SEQ ID NO: 3 - DNA, 25 bp, Homo sapiens
  source 1..25: mol_type = other DNA
ctgcaggaaatggtcaagctagggt

SEQ ID NO: 4 - DNA, 25 bp, Homo sapiens
  source 1..25: mol_type = other DNA
gaagtggtgagtgacgctcctgcag

SEQ ID NO: 5 - DNA, 25 bp, Homo sapiens
  source 1..25: mol_type = other DNA
gttaggaagacagaagtggtgagtg

SEQ ID NO: 6 - DNA, 25 bp, Homo sapiens
  source 1..25: mol_type = other DNA
atcctggcaaaaactgttaggaaga

SEQ ID NO: 7 - DNA, 25 bp, Homo sapiens
  source 1..25: mol_type = other DNA
gtgggattcaggagcccgcaggatc

SEQ ID NO: 8 - DNA, 25 bp, Homo sapiens
  source 1..25: mol_type = other DNA
ggtgacattgtgggattcaggagcc

SEQ ID NO: 9 - DNA, 25 bp, Homo sapiens
  source 1..25: mol_type = other DNA
ggagccaaaggtgacattgtgggat

SEQ ID NO: 10 - DNA, 25 bp, Homo sapiens
  source 1..25: mol_type = other DNA
ggtcacaaaggagccaaaggtgaca

SEQ ID NO: 11 - DNA, 25 bp, Homo sapiens
  source 1..25: mol_type = other DNA
acagtgcagggtcacaaaggagcca

SEQ ID NO: 12 - DNA, 25 bp, Homo sapiens
  source 1..25: mol_type = other DNA
ctgttgctgtacagtgcagggtcac

SEQ ID NO: 13 - DNA, 25 bp, Homo sapiens
  source 1..25: mol_type = other DNA
gggacaggaatgcctgttgctgtac

SEQ ID NO: 14 - DNA, 25 bp, Homo sapiens
  source 1..25: mol_type = other DNA
caggtgatggtggggacaggaatgc

SEQ ID NO: 15 - DNA, 25 bp, Homo sapiens
  source 1..25: mol_type = other DNA
ccgttttcaatccaggtgatggtgg

SEQ ID NO: 16 - DNA, 25 bp, Homo sapiens
  source 1..25: mol_type = other DNA
tgacactcacagcatttccgttttc

SEQ ID NO: 17 - DNA, 25 bp, Homo sapiens
  source 1..25: mol_type = other DNA
aagtccccacacacatgacactcac

SEQ ID NO: 18 - DNA, 25 bp, Homo sapiens
  source 1..25: mol_type = other DNA
ggtcttccccagacaagtccccaca

SEQ ID NO: 19 - DNA, 25 bp, Homo sapiens
  source 1..25: mol_type = other DNA
actatgtcagtagatttgaagggaa

SEQ ID NO: 20 - DNA, 25 bp, Homo sapiens
  source 1..25: mol_type = other DNA
tcccactatactatgtcagtagatt

SEQ ID NO: 21 - DNA, 25 bp, Homo sapiens
  source 1..25: mol_type = other DNA
tcagtcaaggatttcccactatact

SEQ ID NO: 22 - DNA, 25 bp, Homo sapiens
  source 1..25: mol_type = other DNA
aaaagaactcagtcaaggatttccc

SEQ ID NO: 23 - DNA, 25 bp, Homo sapiens
  source 1..25: mol_type = other DNA
gtaaaggaaaataaaagaactcagt

SEQ ID NO: 24 - DNA, 25 bp, Homo sapiens
  source 1..25: mol_type = other DNA
aacctgacagagtaaaggaaaataa

SEQ ID NO: 25 - DNA, 25 bp, Homo sapiens
  source 1..25: mol_type = other DNA
ggacccagaagaaacctgacagagt

SEQ ID NO: 26 - DNA, 25 bp, Homo sapiens
  source 1..25: mol_type = other DNA
cactctcttgaatggacccagaaga

SEQ ID NO: 27 - DNA, 25 bp, Homo sapiens
  source 1..25: mol_type = other DNA
cactcggtctttcacactctcttga

SEQ ID NO: 28 - DNA, 25 bp, Homo sapiens
  source 1..25: mol_type = other DNA
gtcttgagtcaatcactcggtcttt

SEQ ID NO: 29 - DNA, 25 bp, Homo sapiens
  source 1..25: mol_type = other DNA
gataaacagctgcagtcttgagtca

SEQ ID NO: 30 - DNA, 25 bp, Homo sapiens
  source 1..25: mol_type = other DNA
agtcctggcttggtgataaacagct

SEQ ID NO: 31 - DNA, 25 bp, Homo sapiens
  source 1..25: mol_type = other DNA
atgtgtagagtcctggcttggtgat

SEQ ID NO: 32 - DNA, 25 bp, Homo sapiens
  source 1..25: mol_type = other DNA
gtagctatgcatgtgtagagtcctg

SEQ ID NO: 33 - DNA, 25 bp, Homo sapiens
  source 1..25: mol_type = other DNA
tgcttattggtagctatgcatgtgt

SEQ ID NO: 34 - DNA, 25 bp, Homo sapiens
  source 1..25: mol_type = other DNA
acttctccccatgcttattggtagc

SEQ ID NO: 35 - DNA, 25 bp, Homo sapiens
  source 1..25: mol_type = other DNA
ccttggcagtactgaacttctcccc

SEQ ID NO: 36 - DNA, 25 bp, Homo sapiens
  source 1..25: mol_type = other DNA
cctgctatgctgatggtggctgcag

SEQ ID NO: 37 - DNA, 25 bp, Homo sapiens
  source 1..25: mol_type = other DNA
gggcatcctacctgctatgctgatg

SEQ ID NO: 38 - DNA, 25 bp, Homo sapiens
  source 1..25: mol_type = other DNA
gcaaatgtgaaggggcatcctacct

SEQ ID NO: 39 - DNA, 25 bp, synthetic construct
  source 1..25: mol_type = other DNA
tgcatttctaaaagaccaccctagc

SEQ ID NO: 40 - DNA, 25 bp, synthetic construct
  source 1..25: mol_type = other DNA
tctaaaagaccaccctagcttgacc

SEQ ID NO: 41 - DNA, 25 bp, synthetic construct
  source 1..25: mol_type = other DNA
accctagcttgaccatttcctgcag

SEQ ID NO: 42 - DNA, 25 bp, synthetic construct
  source 1..25: mol_type = other DNA
ctgcaggagcgtcactcaccacttc

SEQ ID NO: 43 - DNA, 25 bp, synthetic construct
  source 1..25: mol_type = other DNA
cactcaccacttctgtcttcctaac

SEQ ID NO: 44 - DNA, 25 bp, synthetic construct
  source 1..25: mol_type = other DNA
tcttcctaacagtttttgccaggat

SEQ ID NO: 45 - DNA, 25 bp, synthetic construct
  source 1..25: mol_type = other DNA
gatcctgcgggctcctgaatcccac

SEQ ID NO: 46 - DNA, 25 bp, synthetic construct
  source 1..25: mol_type = other DNA
ggctcctgaatcccacaatgtcacc

SEQ ID NO: 47 - DNA, 25 bp, synthetic construct
  source 1..25: mol_type = other DNA
atcccacaatgtcacctttggctcc

SEQ ID NO: 48 - DNA, 25 bp, synthetic construct
  source 1..25: mol_type = other DNA
tgtcacctttggctcctttgtgacc

SEQ ID NO: 49 - DNA, 25 bp, synthetic construct
  source 1..25: mol_type = other DNA
tggctcctttgtgaccctgcactgt

SEQ ID NO: 50 - DNA, 25 bp, synthetic construct
  source 1..25: mol_type = other DNA
gtgaccctgcactgtacagcaacag

SEQ ID NO: 51 - DNA, 25 bp, synthetic construct
  source 1..25: mol_type = other DNA
gtacagcaacaggcattcctgtccc

SEQ ID NO: 52 - DNA, 25 bp, synthetic construct
  source 1..25: mol_type = other DNA
gcattcctgtccccaccatcacctg

SEQ ID NO: 53 - DNA, 25 bp, synthetic construct
  source 1..25: mol_type = other DNA
ccaccatcacctggattgaaaacgg

SEQ ID NO: 54 - DNA, 25 bp, synthetic construct
  source 1..25: mol_type = other DNA
gaaaacggaaatgctgtgagtgtca

SEQ ID NO: 55 - DNA, 25 bp, synthetic construct
  source 1..25: mol_type = other DNA
gtgagtgtcatgtgtgtggggactt

SEQ ID NO: 56 - DNA, 25 bp, synthetic construct
  source 1..25: mol_type = other DNA
tgtggggacttgtctggggaagacc

SEQ ID NO: 57 - DNA, 25 bp, synthetic construct
  source 1..25: mol_type = other DNA
ttcccttcaaatctactgacatagt

SEQ ID NO: 58 - DNA, 25 bp, synthetic construct
  source 1..25: mol_type = other DNA
aatctactgacatagtatagtggga

SEQ ID NO: 59 - DNA, 25 bp, synthetic construct
  source 1..25: mol_type = other DNA
agtatagtgggaaatccttgactga

SEQ ID NO: 60 - DNA, 25 bp, synthetic construct
  source 1..25: mol_type = other DNA
gggaaatccttgactgagttctttt

SEQ ID NO: 61 - DNA, 25 bp, synthetic construct
  source 1..25: mol_type = other DNA
actgagttcttttattttcctttac

SEQ ID NO: 62 - DNA, 25 bp, synthetic construct
  source 1..25: mol_type = other DNA
ttattttcctttactctgtcaggtt

SEQ ID NO: 63 - DNA, 25 bp, synthetic construct
  source 1..25: mol_type = other DNA
actctgtcaggtttcttctgggtcc

SEQ ID NO: 64 - DNA, 25 bp, synthetic construct
  source 1..25: mol_type = other DNA
tcttctgggtccattcaagagagtg

SEQ ID NO: 65 - DNA, 25 bp, synthetic construct
  source 1..25: mol_type = other DNA
tcaagagagtgtgaaagaccgagtg

SEQ ID NO: 66 - DNA, 25 bp, synthetic construct
  source 1..25: mol_type = other DNA
aaagaccgagtgattgactcaagac

SEQ ID NO: 67 - DNA, 25 bp, synthetic construct
  source 1..25: mol_type = other DNA
tgactcaagactgcagctgtttatc

SEQ ID NO: 68 - DNA, 25 bp, synthetic construct
  source 1..25: mol_type = other DNA
agctgtttatcaccaagccaggact

SEQ ID NO: 69 - DNA, 25 bp, synthetic construct
  source 1..25: mol_type = other DNA
atcaccaagccaggactctacacat

SEQ ID NO: 70 - DNA, 25 bp, synthetic construct
  source 1..25: mol_type = other DNA
caggactctacacatgcatagctac

SEQ ID NO: 71 - DNA, 25 bp, synthetic construct
  source 1..25: mol_type = other DNA
acacatgcatagctaccaataagca

SEQ ID NO: 72 - DNA, 25 bp, synthetic construct
  source 1..25: mol_type = other DNA
gctaccaataagcatggggagaagt

SEQ ID NO: 73 - DNA, 25 bp, synthetic construct
  source 1..25: mol_type = other DNA
ggggagaagttcagtactgccaagg

SEQ ID NO: 74 - DNA, 25 bp, synthetic construct
  source 1..25: mol_type = other DNA
ctgcagccaccatcagcatagcagg

SEQ ID NO: 75 - DNA, 25 bp, synthetic construct
  source 1..25: mol_type = other DNA
catcagcatagcaggtaggatgccc

SEQ ID NO: 76 - DNA, 25 bp, synthetic construct
  source 1..25: mol_type = other DNA
aggtaggatgccccttcacatttgc

SEQ ID NO: 77 - DNA, 144768 bp, Homo sapiens
  source 1..144768: mol_type = other DNA
tatacagtcatttatcacttaacatcagggatatgttctgagaaatgcatccatagttcattttgttattgtgtgaacatcatagagtatacttacagaaaccttgatggtatagcccactgcacacctaggctgtatggatagcctattgcccgtaggctacaaacctgtcagcatgttactacactgaatactgtaggcaattgtagcacaatggtacgtatttgtgtatctaaacatagaaaacatacagtaaaaatacagtataaaagataaaaatggtacacctgcattgggcacttaccatgaacgaagatttcagcactgaagtttgctctgggtaagtcagtgagtgagtgatgtgtgaatgtgaaggcctaggacattactgtacactgctgtaaactttataaatactgtacacttagactacgctaaatttatttttaaaatatttttctttctttaataataaattaaccttagcttaccacaactattttattttataaacttttaaaatttgtaatctttttgactcttttgtaataatactctgtttgaaacacaaatatattgtacagttatacaaaaatagtttttcttcatatccttaagctcttttctatcttaaaattttatttttattttttactttctaaactttttttgttaaacactaagacaaaaacacacaatagccttggcctacacagggtcaggatcaaaacatcactgcctttcaacagcttgtcctactggaaggtcctcaggggcaataacacacatggagctgccatctcctatggtaacaatgtattcttctagaatacctgcctgaggctgttttacaatcaattttttatttatttttattttttatgtttttaaataagtacagggtatacacagtaaaataataataaaaatatagtacattaaaaaaaccagcaacatggtcatttattatcattatcaactattatgtactctacataattgtatgtgcttttttattcaaaaagaaaatcattaagaacacagcaatattaatatttattcttttttttagaagaacctgatttatttatttaatttttttattatactttaagttctgggatacatgtgcaaaacgtgtaggtttgttacacaggtatacatgtgccatggtggtttgctgtatccatcaacccatcatctacattaggcatttctcctaatgctatccctcccctagccccccaccccttgacaggccccagtatgtgattctcccctccctgtgtccatgtgttctcattgttagctcccacttatgagtgagaacatgtggtgtttggttttctgttcctgtattagtttgctgagaatgatggtttccagcttcatccatgtccctgcaaaggatataaactcattgttttttatggctgcatagtattccgtagtgtatatgtgccacattttctttatctagtctatcattgattggcatttgggttggttccaagtctttgccattgtgaatagtgctgaaataaacatatatgtgcatgtgtctttatagtagaatgatttatagtcctttgggtatatacccagtaatgagattgctgggtcaaatggtatttctggttctaatccttgaagaattgccacactgtctcccacaatggttgaactaatttacactcccacaaacagtgtaaaagcattcctatttttccacattctctccagcatcggttgcttcctgactttttaatgattgccactctaacaggcatgagatggtatctcattgtggttttgatttgcatttctctaatgaccaataatgatgagctttatttcatatgtttgttggctgcataaatgtcttattttgagaagtgtctgttcatatccttcgcccactttttgatggggttgtttgtttttttcttgtaaatttgtttaagttctttgtagattctggatattagccctttgtcagatggatagattgcaaaaattttctcccactctgtaggttgcctgctcactctaatgatagttccttttgctgagcagatggtctttagtttaattaggtcccatttgtcaattttgacttttgttgccattgcttttggggttttagtcatgaagtctttgcccatgcctatgtcctgaatggtattgcctaggttttcttctagggtttttatggttttaggttttacatttaagtatttaatccatcttgagttaatttttgtatgagatataaggaaggggtccagttccagttttccgcatatggctagccagttttcccaacaccatttattaaatagggaatcctttccctactgcttgtttttgtcaggtttgtcaaagatcagatggttgtagatgtgtggcattatttcttaggcctctgttctgttccattggtctatatatctgttttggtaccagtaccatgctgttttggcaaaccaggaagaagctgaatccctgaatataccaataagaagttctgaaattgaggcagtaattaatagcctaccgagcaaaaaaaaaaaaaaaaaaaaaaaaatccaggaccagacggattcacagccgaattctaccagaggtacaaagaggagctggtaccattccttctgaaactattccaatcaatagaaaaagagggaatcctctctaactaattttatgagtccagcatcatcctgataccaaaacctggcagaggcacaaccaaaaacagaaaatttcaggccaatatccctgatgaatattgatgtgaaaattctcaataaaatactggcaaactgaatccaacagcacatcaaaaagcttacccaccacgatcaagttggcttcatccctgggatgcaaggctggttaaacatatgcaaatcaataaatgtaatccatcacataaacagaaccagtgacaaaaaccacatgattatctcaatagatgcagaaaaggctttcaataaaatttaacacccctttatgctaaaaactctcaataaactaggtattgatggaacccatcgaaaaaaaatgagctatttatgacaaacccacagccaatgtcacactgaatgagaaaaagctggaagtattccctttgaaaactggcacgagacaaggatgccctctctcaccactcgtattcaacctactattggaagttctggccagggcaatcaggcaagagaaagaaataaagcgtattcaaataggaagagagaaagtcaaattgtctctgtttgcagatgacatgattgtatatttagaaaaccccatcatctcagccccaaatctccttaagctgataagcaacttcagcaaagtcccaggatacaaaatcaatgtgcaaagatcacaagattcctatataccaataacagacaaacagagagccaaatcatgagtgaactcccattcacaattgctacaaagagaataaaatacctaggaatccaacttacaagggatatgaaggacctcttcaaggagaactacaaaccactgctcaagggaataaaagaggacacaaataaatggaaaaacattccatgctcatgataggaagaatcaatattgtgaaaatggctgtactgcccaaggtaatttatagattcaatgctatccccatcaagctaccattgactttcttcacagaattagaaaaaactactttaaatttcatatggaaccaaaaaagagcccatatagccaaggcaatcctaagcaaaaagaacaaagctggcggcatcacactacctgactctaaagttctataaatgaactcacgaggccaaagaatgaagaagacaccaacacaaaggggaaaaaatcaaaacaatatggtcattgcaatggtagagatatgcacagtatattatggaaacccagagaagacaaacgtaacacagctttgggaagatatggaatttttctcccaggaggagaggatgatcaagctgagagatgtgtgaactgagtggaaaggaggggagtagtattccaagagaagcaaacagtatgagcaattcatggaggtaggaaagctgtatacagggaaccatcagcaggttgttgaataaaggaagctgaatgaggctgggcacagtagctcacgcctgcattcccagcactttgagaggccgaggtggaatcatcatttgaggccagcagtttgagaccagcctgggtaacatagcctgaccacatctctccaaaaaataaaaataaattagcctgctgtagtggcagtgcctgtagtcctagctactctgaaaggatcacttgagtccaggaggctgaggttgcagtgagctatgattaccccattgcactccagcctgggcaacagagcaagaccctgactctgaaaaaaaaaaaaaacaaaaaacaaaaacaggatcgtaaaagacaatactaacatacaacaaagcatgcagattccagtaaagcctgtcatattaggcagattaggcaggtttaaatatatttttatgtgatgatgagcccctaaaggcttttgagcaggagggactatctgaaaaaagatatagaatggttaagacccaagacagagagaaggtacaaggactatttggttgataaaatctgtaggaattggaatgggctgaatgtgggagtgggggtggggtgagagatggggaggactgaagactgaatagacccagatttctggtttgagtccgggagaggtggggtgctgaattcgaaggtcaggacacctatacctctgggaatacagcgatggttgcaaaaggcatctggtattgtccttctgccactgtcttaaaagggattctaaagggattttcttgcccttgttcctccagccctacacttgcattactggcctacgtggatgctcaccagctggctgtgtccatcattgcagccaagtctgagaattcctgattatacttagagcctgtggagctgctgacacaaacagtcattagcagacaacccttttgcaacaaagtatgctttaaaatgtaaactgtggagccattttccttgcgttgtccagaaggaacttcgtcctgcgtgagcctggattaatcatgagagagctcgtcaacattccactggtacatattcttactctggttgccttcagcggaactgagaaacttccaaaaggttggtttgagcaatcgtgtcttcttgttgtcttgtcatggttgtaaagtgtgtgtatatgagatatgaccaagactgatttatagtatggtgggcttgggttttacttgaagaagtaagggtgaaatgtatgatgagggaggaaattatatacataagcaattggttgatggtctctctaccaatattttactccgaagttctgcaataaaaggcttagccacatagtgcatgcagagctctgacttctagggcaaaacataagcatgtacactgcaagttatcaaggtctattttgtttatgcgaatgtcaaatttcagggtggtctaagtggatgatgacaaccttttcgcatcaggtttgccaggaatagaatcaaccccattctcagacatccagagcatttgcttctgaataaaaatcacttttattttcagttttagctttgccaaactcttaataaatattttagttgctaaaaattggaaatacagaaagacgtacacaatttgcctgtgtaacaaacccggaagacaatggattttaaataactttgaggctggagtcatgatttggcttggcaagagactgcaaaagtttttttcagagcctttggaatgtaccggaaataacaccacctcaggggaaaggaaagcaacttaagaaactgacaaacacctgttgcagtagcaagaatgtccagtgatatagtttctggccagtgtattatgaaactgggactagcgtatttgaaatcagaaagactggaaagtctgtgatttgcaaagtgactaagggtatcactgaaatctttttttaaaaagacatgagaaagcagatcgagtggtatcaatacaagagttttcaaatttaatgttataccatagagtaaaaagagatttttgcttcaggacatgttcaaattaatcattttccctttatattaatcaaagcactgaaacagtttgtattgtgtagataaatgaaggtggtatctgtatatagatgcaaaatgtatctttagattttctggttcactaaaaattctggagccataaggtcaaagttcacatgcaaataagatgcgggtttcttcacaaacaatctgatttcacagatttttttgtttcattttgttgcttctctttttccattttacttgagtataatggtatcttcaaatgtatttaaacatttaatttctaatgaatactttaaggcatctacagataaaagagactctagatcttttcaaacagctaataatagtcagaaagacttaacattggcttcagattattttaatacttcctgatcaaacaacggatgtccggtaaggccccacctggatgtttgaacgtatgggttgtcttaagtattatttgtatatttctaaaccacgcagttacataaaaatgatagggttaaatactaaaatacagtcttataaagagcaggacaattgattttgatgattcaaataaggatttgttgtacacacacacaaaacccccaaaatctctctatctgaataagtcttaatttatattctagaaagaattgattttcatttccaaactttacaaataaagcccttaattcaaatgcacactatctctattttgaaaatattgaaatatagtggcatagagataacaaagccaaacaccctgacttttaaataaactaaccagatcattcacccaatttcatttcttataatttatttactactagaattaggttttaaaatcaaaaaatttaagcaattttttctcagtctattgtactgtaaaatctaaattagtatgagtttaacatcactcttagccaaaataccatgtctaggagaacaaacatttatttactacatacagcagttctgagaataactaatatcatgaaagcataatccttaaaattttaaatgttgaagggtgaggaatacattggtagaaaagaaccacaataaaattttattattttcattattattattattatttgagacagagtctcgttctgttgccaggctgcagtgcagtggtgtgatctcagctcactgcaacctcctcctcccaggttcaagctatactcctgcctcagactcgcaaatacctgggactacaggtgcaagccaccacactcggctaatttttatatttttagtagagacagggtttcaccatgttggccaagatggtctccatctcttgacctcgtgatctgcctgcctcggcctcccaaagtgctgggattacaggcatgagccactgcgccagccattatactactgaaactaataaaaatggcccacccaaactatttctactatttctagaaaactactgttttccctattcttggcagacggtaataacaaaaagagttttttagataatatacttcttcctaagtcctttatcatgaatgcataaaggaaattgtacaatatgtattagctaataaattgacatatttaaaataacacaaaagtattcagtggattattttctgactttctgcacatcattttttgataaaaattaatatccaactttgtagaattaattgttcatagttttcattttagtttaattaaggctactaatgcccaatgataaggggaagaatcattgtctttctgaatcatttcaactagtgatattttcaatgatgaattcagtgccataacccaagtagtagaatttcacaaactgtcatttatgaaagtagccaatgttccactggataattggcttaactacatacatcaataacatttaatgctttattagggagtattaaatgtatggtgcaatgttagattctgcaggagcataagagttcatgtaagcagctctggatttgcttaatatgcacaaagaactatcaaaagtctcactttcaaaacacccgacttactacaatacgttgaagtgtttgagcgtacgtgattttatctttagatagatttgaataacctcctcttttaaattgtatgtatataaggataggattcacagatttgacatatgactaagtcatgactgacacagctgtcattgaaatccaggacacacagtcctggatgaatattttatattgacatgcattgaaagagttactcattgcatgtttgagtaaacagagttgcttcaaatattattgaccttaaagaaaagaaggaaggagcagaaatttcaagatctctttaaactctaccttatttccaaaggtaaatgaagtaggcagcttctcagtgaaataaaacctgatgtcgttgaaggagagtaacaagtgaggggtggaaagagggcctgaaaggagtggctctattcaggaagatgagaggatcacatgggccatgctgaagacccagctggggctaggaactgcagctttctttatagtgcccctgacctgaacatttactgcactatacccagcagtgctatgtgaacatagggtagggaggacagatggctggattaattcaggattggcactttccagggtcagtacagctaaagaactagagagtaaaggagttggagttatatggcagcgtaccatgagatacagattggatcaggaagaaagtaaaacaaggatatttaatgcttggaagagaaggttaaaaagtttaaaccaaccccaaagtgttttgttgtactaaaaaaaaaatactgcattcataatgttagagaaacatgaatattttccaagtcagagccctaattttactaaaatagaaaggagcaaatacaccttgtttctttatgtacccaatcattgggtcatgtcaatccaaaaataatcaatcaaaagttggtaagtcaatagtcatatgcgagagcccataaaatacaaaggaaaggtaatgtcatgtccttctcatcaagcagttgatatccaagtcaagcttttcttcgggtccagaaaaggattgtgttcattttcatgtatttgttattcatgtagtccttggattgagagaagaaatagtgccctagtttaaacgagctctggctgagatgaaagctggatagtagaatgaaagtaaacaagagacagtgtgctgagtcaacaaatatcattaaatacaactgatccatgtgattgtgtcagtgaaaaccggaaagcagatttggataactggaaatatggacaatgtggacttggcattctgtgtgtagaaactgatattgatgtacatcttctcttatccatctgtgcataatcagtatgatcttgaagatgcagtatgtcttagagaaaggaagtataaaccaactttaaccctgaagttcagctagttcagctctatcacttcctatttttgtgatatgtagacaagttgtgcaaccatttttagtccccatttctggtctgcaagtggaaatattaatagtatctaccttttttgtgttaaataaaaagataatgaataaaaagtataatgcatggcacataccagattcttaatcacttttagatgctaatattagtaaaaacagtcaaacttcacacaagactgttatgtcactgaatcttatgggaaggtgttaacttaataaagttcttagtaacaccttccatatttctccatgtctattttccaagacattctaagactcacttctttgatcttaactgatctcaacagttcatgtgacatctccagtgtgtcacctcattggaaagataacattaatagtaagtctattacctctgccacttctgcatctgtgtattgagtgaaaataacactagtgcataaagtttctctcttataaaagtagcacatggtatttaagattgattttgaaaatgcaaaatacataaagtttaagacagaagaagtcatccatacttccaggactattaacacattggcatattttcttccatttttttctacatatttttaaagtccttattaacattgtgctgcctgctatttttacttagcactaaagcatgagcatattctatattattagtaatcttccaaacgtcatgtgtggtgactaaataatattctgtttagataacagtgtgctagtatatgtttaatattcacctcactgagaaaaaaaaagacgtggatttgttgctcttgtttatttctgcagtaaaatactctatagtaaaaaactctattgtgtctgtggccagtctcaagctactatcaaataatgagcactgagttgggaaatagcatgtgtagtagcatccttcatatattatttctaccacacagacacattacagataacctcaagaacttatataataataaaatgtagttaaatcattgggaatttcaagtattaatactttgtttttaatatatttgattataaatgtatacaacttagtttttaataatgattgtatataacaatcacctctcaaaattcctaaaaaattaccaagtggctcttgcaaacctgcggaagacaaaacttgtgtgcctcaatcttctcccacttgactacgattaatagaatttagtacaattgatctgttttctgcagtaaaagtcacctatatgcctttctatatgtgagattatttccttaggatagattcccagagatgatgatgatgatgatgatgatattattattattttagagatagagtcttgctctgtcacacaggctggagtacagaggtgcaatcacagctcactgaaactttgaactcctaggctcagttgatcctcccacctcagcttcttcccaagtagctgggattacaaagtgtgtgccaccatgccggctaatttttaaattttttgtagagatgtggtttctctgtgctgcccagcctggtctcaaactcctaggctcaagtgatcctcccacctcggcctcaaaagtgctgggattacagacatgagccaccaaccacacccagcctgagataaaatattaacggtataatacatttgaatgtaataaagacaaggaaaacaatcaaacagcctgggtttctttttctaaattatagaatcaagtaacatttgcacagtgctccatgtggaccaaggacaattttaaatggtttccatttatttattccttgtgacaagttatgtggaggctatacattatctccgtctatagctgagacactgaggttaagtgtggttaagtgacttgcagagtcggtaagtgatggagctgggatgtgaattcaagtcctgtgccacattgcctcttttttttttttttttttttttttgagacagagtcttgcactgtcgcccaggctggagtgcagtgtcgcgatctcagctcattacaagctccgcctccctggttcacgccattctcctgcctcagcctcccgagttgctgggactacaggcacccgccaccacgcccggctaattttttgtatttttagtagagatggggtttcaccctgttagccaggatggtctcgatctcctgacctcgtgatccgcccgcctcggcctctcaaagtgctaggattacaggcgtgagccaccgtgcctggccgctacattgcctcttaatgtgtagttgctcaatagtcttccctttttttttttttttttttttttttgggactgagtctcactctgttgcccaggctggagtgcagcggtgcggtctcggctcactgcaatctccacctcctgggttcaagtgattctcctgcctcagcctcccgagtagctgggattacaggcctgcaccaccacacccagctaatttttgtatttttagtagagatgggatttcaccatgttggccaggaatctcttgaactcgtggtcctccccgcctcggcctcccaaagtgctcggattacaggcgtgagccaccgcacctggcctagtctttctttttaaacagtaaaagcattcaacatcatcaattaacctttgggctcattgttaaccacactccacaagcttaataaaatattgtttttgttctttatctctcaatatctagatgattatactattaattgcttatttcattcattcgttgtttagaaaataactttttattgagattgactttctcaaactttatgaattataagtttgcctccattgcattttgaaaattaacacttctttggcattctatctgctacaatttttttctattatgaaagcaatatgtattcatttttttaaaaaatcctgtaagatacagaaaaacaaaaatataaaattacacataatgctcatagcaagggataagtgctcttagcattttgctattcattctttcagtctctttctctcatatatgtgtgtgtgtatatgtaagtgtatatatgttgtatatatacacatacacacacatatattatatattatatattatatatgtgtgtgtatgtgtatgtaattttattttgtaaattttaagttccggggtacaagtgcaggatgtgcagttttgttacataggtaaacgtgtgccatggtggtttgctgcacctgtcacccatcacctaaataactgggctcagcatgtattagctatttttcataatgctctccctcccccaatcccacctgctgacaggccccagtgtgtgttgttcccttccctgtgtccatgtgttctcattgttcagttcccacttataagtgaaaacgtggtgtttgcttttctgttcctgcattagtttgctgaagagaatggcttccagctctatccatgtccctgcaaaggacataatctcattcctttttatggctgcatagtattccatggtgtatatgtatcacattttctttatccagtctatcattgatggtcacttgggttgcttccatgtctttgctattgtgaatagtgctgcagtgaacataggtgtgcatgtatctttacaaatttcagaggctgttatttcattgaggaaagagaagcatttaaataagaagctctctcttcagctgcctaccagcaaaggggaaaaacctacctctctgcatccatcttgtcctccttctccctcactgtgctttaagaggactttctcttcctatccgtggttaacccgtctttcttcctctgagccccacataaatctgctttctcagggacctatttccctcaattatctcctgtctttctccaatgtcctacatttttgcttctactaggtgcttccagtcagcatttaaacccactcttgtcaccatactcacctccagcaccatgcagcccctttcttcccttttattgttaaactctgctgcaaaaagatgtcttccctctcctcttagtttccacttccttatcacaacctaacccccaccactgcaatctggcttccgccaaatctgcaagcaccaaaatcaacatgacctcctcatcaggaaactcagtgggcacttttaggtccttatttaacttaacgtctcggcagcatttgattctccctcttttgtgaaacgtgtgctactcttgggctctgccaatagcctccactggctttgcttacaactctgatttcttcctctcagtctcttttctaaactcttcattctcccacccatcttttataaatgtttgtgttttcttctaattggcttagggcaccagcctccctccgtcttcaggctttcacgggtaccgtttcctctgcctggaacactcttctctctccactgcaccaagccaaatttgaatcatcctgtgaatgtgtgtgaatgtattgtgtagtttttttttattcctagttttatcaagttcagtggttgagaaacattatttatcatgttaaagaaatactattatattccgactctactaatggttttcaagtagggaatggatagttaactctgttactgcctttttgactgtgataggtatcttttaaactatgactatagtaaatcctgtttaaaggacttttctattaaattatccttatatttttgcaaggattttttttctgcctaaatgcatagtaataattttgaaatttatgatttaatttaatttgctttttaaaattttactttagggctttacatcctgcttgacttacattagcattgttttattccaaagattgatttgagaagctttctatactttatattccctatagcttttgtttgtttgtttttgagacaaggtctttctctgtcacccaggctggagtgcagtggtgcaatcacagctcactgcagccttgacctcctgggctcaagtgatcctcctgcctcaggctcccaagtagctgggactacagatgtgtgccacctgtacccggctaatttttttatttttgtattttttgtagagatgcagtcttgccatgttgcccaggctggtctcaaaacacctggatccttcagtggatccttctgggctcaatggatcatcctgcctcagcctcccaaagtgttgagattacaggtatgagccactgcacctggctccttgtagcttttatgtaactggagacttaattctttcttaagagttttttaaaacttatctgaaaaactttggggcattatatttttttcagaagtgttctttgctaatctaatctttttcttctgtgagtattgttgatgattttaccccaccctttactacatgtgaacaattttgtttttaaaattcttaattctttagattttcatctttaaatattcattactgtttagctctgttgaattgtggtccaagaatgtgacatataatctgtttttaaaatttatttaagctttaattttcatgggcatttgaaaatatgtgtcatctatcagggcttacagttgatgtgcatatctgtttaattaattttactaattctgctattctgttttactacatatataattatatttttgtcttttgatctataaacaggattgggattttttgtttttttgctatctttttattgtctatttcttacagattttattatggttagaaaatatagactatataaaatcaattctttgagctgggttaaaatctcctatgtagcttggtatattttctttatgtttgaatgtttcatctatgcatcatttacaaaatgtattctgtaattgtttcattgttttataaatgtacattatatctagtgttaattttgatgtttatatcttctatatctttactgtagttctgtttgatatattagtgtctgaaggagatctcttaaagggttacactatgttaaaacatttctccatgtactcttgtgataattttacttttgggtttgagtccatatttttaaataattaagtttcatgattgtcatatctttttagggcttgttccttttatcattaagcaatgtttctttttttcttataaatgcatatgcccctatattacatttttcttatattactattgctatgtcactttctttgggatatcgtttgctttatacatattttccctcccctttatttttaacatttctgttttattttatttaagtgtgtcttttgtgtttgatagcatctgtcatttcataggtcagcttataccattggcacttactgtgatttctggtatatttggacttgtttcttccatattattttatgtcttctgttaaccatgctttttcttggcttatttattcctcctcttcttcctgtttgtttttgcttttgtttttgattgataaagcctttctggtacctctttttttctctgttgttttggaatttttgaattttatttattttttagtagtaaccattaataaggttttaacataatcatttaaccatagatgcttctttctgtttgaagttattcaatatttctgtcctcttgtttatggcaaagagcttagcattttttaattacttagcattttaaacttctttcctgaactttgatcttatagttgtttgtctagaatttaattccactctttttaaacacaaaaatttgtgattttttaatattccatagtgtgttaaaattgcaatggtatttcaccaccatttatgctattgttttttataactcacccctttctttctggtataaattattccttagtgagtttatttactctagtaactatttcagagaaagtctgtgagtggtaaacactctcaatatttgtttataagtgttctgagtttaaaattattacctaagctagctgtgtaattctaagctaacagttatttttacgcagtcatgtaaaggtattagcctattgccttttggtatcaattgttgttgctgaataatctcaggttacccaaaattggcattcctattaaggaattaaactttttctttctggtagagtttaagattttatttatctttaatttttacagttttaattcaatgtgtccaagtatgaatttctttctttttttctttttttttttttcttctgagacagagtctccctctatcactcaggctggagtgcagtggtgcgatctcagctcactgcagcctccacctcctgggttcaagcgattatagtacttcagcctcccacatagctgggactacaggtgtgcaccaccatgtccagctaaattttttgtatttttagtagagacagggtttcaccatgttagccaggctggtcttgaactcctgagctcaagtgatctgccctcctcggcctcccaaagtgctgggatgacaggtgtgagccaccatgcccagctgtatttcttttttaaatctgtcttgtcatttggtgggtactttcgaccttaatattccagggtttgtttttcagttttgaaaaattatcagccatttatctctttatatattgcttctaggctgctcccctcttttatttttctcaaactcctatcagaggtgtattagtgcctgtcactgtatactttatatctcctaactattctttaatagtgtccatgtctgtatttctttgtattatgttctgtatattataatgatccttatgatatatatatcatatatatgatatatataatgatccttataatatattatattatatataatatatattatatattatatattatataatatatattatatattatatatataatattatatatattatataatatatattatataatatatattatatataatatatattatatatattataaggatcgttataatatatattataaaaattattataatatatattataacaatccttataatatatattataatgattattctgttgaaaagctgggaagaaagtggccctgatgactgtgtataagatgtcattttacataaaaattctgctttcttatttcaactactatgtttcaacttttttatttactccttttgtgaaggccttataaatacagttttgttcatttaaacagtacatcagtcactgtgctcaccctgggaattaaatgatgtctaaaacatgatccctgacctaaagctcaaggttttaataattgagtgtgggtcaaagacgtaaacagatcattttaatagtatctgtgtgacaagtggcattataagaatgacatttcaggataacataaggttaaggaaagaggcatttgggtctgcctagcatgtcaggggaagtggaaggagatggatgcttaaactgaatcgtaaaagacaaaataacattagcaagggagatatggggcaagtaaaacgctcccactcaccattttttttcactctcagcagatgagcttgcctttgactttaccaaaaaataataataataataaggtttattcagaaagtggatgtatcttttatggccaaacaacgtgccagtctgttctagatgctctataagtctgtcactgactatttcacttctaaatgtcaatcatactacttatttggctttgatgtataccttgccctttaactgaagggaccatataatttactgtataaatgcaggtagccctcatttcacagaggagtgagaaaaaagaaaggacgatgtctatagcctttcctcctgttgtcttaaaaaaaataaaatagaacaaaacagctttgagatctaattcacataccgtgcgctttacccattaaagtgtacaattcacaaagtggtgtctcttcattttctagttgtatgcccaagtgaattctctatgaatctgatgaagcttaagcttcagattctctcatttgcaggagccctttctaagaccctcagtgggctttagcaatgtgcttttatgagtgtctatttttataaaattaggggaaaattgttcttaaaaaacaattttttaaattattatccttttccaccctgacttcccttcagactcacttccctttatgctgatgatattggggtggacacagaaattttgtattcataattttatattttttctttaaaaagtacaaattgcacaaacttgggccccataaaacttggatctgcctctcctcgtatatcataaactctaagccctgctctcctgagagacttctcttttgattttcaaatgcctagtttggaagtagaaacaacattctttacataagctcttcctttcttttggcaaatttctactctggggaaacagaattctcattcaggtatgtaatggcttctgactgcttaaggaagggttagtaaacaaaattctagaatgttctcttttgatttctcctttcctttcagctcctgtcatcaccactcctcttgaaacagtggatgccttagttgaagaagtggctactttcatgtgtgcagtggaatcctacccccagcctgagatttcctggactagaaataaaattctcattaagtaagtattccacattttaatttttttaaatttttgtgggtatatagtaggtgtatatatatgtatatatttatgaagtagatgagatgttttgatacaggcatgcaatgtaaaataagtacctcatggagaatggggtatccatcccctcaagcattaatcatttgaattacaaacaatccaattacacactttaagttattttaaaatgtacaactaagttattgttgactatagtcactctgttgtcttatcaaatagtaggactttttcattctttctattttttttgcacccactaaacatcccctactccccccgatccctgtactaccctttccagcctctggtaaccatccttttacgctccatgtccatgagttcaattgttttgatgtttagatcccacaaataagtgagaacatgcaatgtttgtctttctgtgcctggctaatttcatttaacataatgatctccagttccatccatgtctttgcaaatgactggatctcattctttttatggctgaatagtactccattgtgtatatgtaccacattttctttatccattcatctgttaatgaacacataggttgctttcaaatcttagttactgaaaacagtgctgcaacaaacataggagtatagatatctctttgatatactgatttcctttcatttgcttatatatgcagcagtggggttgctggatcatatggtagctcaatttttagttttttgaggaaacttcaaactattctccatagtggttgtaccaatttacattcccacatcaacagtgtacaaggattcccttatctacacatccttgcgagcatttgttattgcctgtcttttggctctaagccattttcactgaggtgagatgatatcttattgtagtttttatttgcatttctctgatgatcagtgatgttgagcactttttcatatgcctgtttgccatctgcatgtcttctttcgagaaattgttcaaacttttttgcctatttttgattggattaatatattttttactatagagttgtttgagctccatatatattctggttattaatcctttgtcagatgggtagtttgcaaatatttcctcccattctatttgttgtctcttcactttgtttattgtatcctttgctgtgcagaaggccttttaacttgatgtgatcccatttgtccatttttgttttggttgcctatgcctgtgggtatctctcaagaaatttttccccagaccaatgtcctggagagtttccccaatgttttcttggagtagtttcatagattgaagtcttagatttaaatctttaatccattttgatttgatttttgtatatggagagagataggggtctagttttattcttctgtacatggatatccagttttccacacatcatttactgaagagactgtctttttcccagtgtatgttcttggtacctctgtcaaaaatgaaatgagttcactgcaggtgcatgaatttatttctgggttctctgttttttcccattggtctatgtgtctcttttgatgaaagtactgtgctgttttggttactctagctctgtagtatgatttgaagtaaggcaatgtgattcctccagtttttttcttttttcttttggctattctgggcctttcgtggtttcacataaattttaggattttttttttctatttctgtgaagaatgtcatcggtattttgataggaattgcatggaatctatagactgcttttggtagtatgaacattttaataatattgattcttccaatccattaatatggaatatttttccatttttttggtgtcctcatgaatttctttcattgatgtcttacagttttcattatagagatctttcacttctttgattaattcctagctatttaattttatgtgtggctattgtaaataggattatttttaaatttatttttcatattgttcattgttggtatatacaaatgttactgatttttgtacattgattttgtatcctgcaacttcattgaatttatcagttctaatagttttctgtggagtatttaagtttttccaaatataagagcatatcatttgtaaacaaagataattcttcccttccaatttggatggcccttaatatctttctcttgtctgattgctctaggtaggacttccaggactgtgttgaattaacagtggtgacagtgagcatccttgttgcattccagactagaggaaaggctttcagtttttccccattcagtatgatactactagctgtgggtctgtcatatatggcttttattattttgaggcattttccatctattttttgagggtttttatcacaaagggatgctgaatttttatcaaatgctttttcagcatatattgaaataatcgtatgtttcttacccttcatctgttgatatgatgtatcacattgattgatttgcatatgttgaactatccttgtatcccagggataagtttcacttggtgatgatatatgatctttctaatgtattgttgaatttggtttgctagtattttgtgcagtctggataggccaataatcccaaatcatcaagtcctggttccttttttgcttagcagtttttcccacttatcttttcctcttgaattttactataaccagaaaaaagaaatgaggccgcagcttcgacacatttcttagaaatttcctcagctaaatatctgagttcatcattcacaagctttgttttccacataactgcaggacaccattcagctaagttctctgtcctgcataacaaggatccccctttcttcttttcccagtagcatgctcctcatttccactcaagtcctcaccagtagtgctttgaacatacatatttctacgaatagtctgttcatgataatttaggtattctttaagatgatgtagattttctctaccatggaactctcttccttccaattcctcaggagcagaatcattaacacacgtatttctattaacagtctctttgaggaaatacaggctttttctagcatgcttttcaaatttttttcggttctaactattcatcaattgcaaagccacatccacatgtttaggtatttgttgcagcagcaccacacttccaggtaccaaaacctgtatttatttattatccttgctgtaacaaattaccacaaacttagtggcctaaaacaacataaacgtactatgttacagttctggagttcacacgtatgaaataaatctctctgggctaaaatcaaagtgttggcagggctgagttccttctggaggctctagggaagaatctgatttcttgcctttcctagcttctagaggccacctgcgttccttggccccctactccacttataagtctagcaatggcactactctgacctctgcctccattgtcatacctccctttatcaatctgcctctctcttccatcttttaaggatacttattatcacacagggaccattcgaatagtccagaacactccattttaagattcttaacatctgcaaaataaggtaacatattgacagattttggtgattaaaacaagaacctctgtgggagtgttattctgcttaccacacgcatttggcataaaaaaactcttatttggttaaaagaaactctcttttatattgctgcttctcttcctatttctttctactgccctaattcccttctcaacaaatggtttgcatataattaaataggtaatatatgtaaagcatatatttttatagttggaaattgttttcctcccttatgtatcccaagtaactagaagtgtgcctggcacttgttagcaatcagtaagtaattgtttaatgaaagaataaatattcaactaatcattagttacttctataagtaattaaccctttctgttcatcgtatttatacatatgcatgttctttatctattaatagctgctacttcccagaacttattgggagaagacacctatgtcttggttttatatgcttttctgaataaagtaaactttacattttgtcttatcagtgggcatcaccctttgaaagcatttttgaaggactatggtactttgactattggagcctaagaaagatgagaatataatgttagctttatattatcaagtaaggttattctattattcagaagtcactcaagtttcctctcacctttattctacttcctcattaacaagtcatcggtttgatacattaatcatggcaaaaaaatcacagaggaagcactaatctgtcatttttttctgtgacctgcagactctttgacacccggtacagcatccgggagaatgggcagctcctcaccatcctgagtgtggaagacagtgatgatggcatttactgctgcacggccaacaatggtgtgggaggagctgtggagagttgtggagccctgcaagtgaagatgagtgagtgggaaacagattcgtctattgatttgaaagttgacttggtacacttagttttgtatttattttttacattttttttttgagacagagtctcactctgttgctcaggctggagtgcagtggcatgatctcggttcactgcaacctccacctcctgggttcaagtgattctcctgcctcaggctcctgagtagctgggactacaggtgcgtgccaccaagctcagctaatttttctattttcagaatatacagggtttcactatgttggacaggctggtctcgaactcctgacctctggtgatccgcccaccttggcatcctgaagtgctggaattacaggcgtgagccactgtgcccccgctgacttggtacactttaaaacagtcagtaaatagagattttctcaggcctggtgattcagcactgagacaagttcaagttcaagcacaaccctgaggccaattaatcccacacctcttatgcctgctatcaaccactaccctaaggcaagcctctcaattctctatcctctcccattttcttaccttcactcatctcctgacttcttccaccctgaattctttcaccaggaaaacttcctctgaaacatttaccctacacctgtgaaacacaaccaaacacactttttatttctaacctctttgcagacacttttgccttatttcaaatttgatattcttctgagtgattttctgcttcagtggaagctgttctattttctccacttcatgtctacaagactgccaaacactagtaggtaatctcccagccctcagtgttgcttttaaaccattctattttgtattcctatacaaaatctttcctctgatgagggcttatgccattctgacctatcttttctctaactgtattcatcatgtcatctgctcttctaggcacttccccatatttattaaaaactttgattccaggcttactatctttctatccatctctactcctgcaattaccctaagaaattttaacagccatgaaatagaaccactagccacttaattctcataattccttgacctgcagaaatccagtgatgtttgtctttttttttcaattttaaggctcatacttttaatctctatgtacacacgtatgtagtcgttaaggtaagtctgccactgtgaggaaaggtagtttcattttatttttttaactttcattttgagttcaggggtacatctgcagaatgtgcagttgttttacataggtaaacatgtgccatggtggtatactgcacagatcatcccatcacctaggtattaagcctagcatccattctattcttcttgatgttctccctccccacaccaacacccccgacaggccccgtgtgtgctgtttccccccatgtgttcatgtgttcccatcgttagctcccacttctaagtgagaacatgcagtgtttggttatctgttcctgcattagtttactgaggataatggcttccaactccatccatgtccctgcaaaggacatgatttcattccattttatggctctgtagtattccatggtgtttaggtaccacattttctttatccagtccatcattaatgggcatttaggttgtttgtgttaactgttttattgtttttttgttatatatatttatatataaatatatgtaaacatatatttaaatataaatacatacatatatttaaatatatacaaatatatacttaaatatataaaaacatatttaaatataaatacgtataactatatatttaaatatataaatatatagctatagttatatatagttatatatttacatttaaatatatagttttataatttatttatataaactacataaataaactatatatttatataaaatataaataaactatatatttatataaatatatcatatatatattcatattttatataatatataatatataaatatataaatatataaactatatatttatatattgtatagttatatttatatataaatatatagctatatatttatatttaaatatatattaaatatatatttaaatataaatatataaatatatataaatacatatttatatatatgtaaaaaatataaaaatatgtgaaaaatacatatttatatataatatatgtaaaaaatattaaaatatgtaaaaaatacatatttatatatatgtaaaaaatataaaaatatgtaaaaaatacatatttatatataaatatatacaactatatataaatatatagttatatatatttatatattatatatatttatatattttttactacataatatataacatattatatattttagtatatatattatatattagtatatattagtaatataccatataatatacatagtatattatatataactatatatagttatatataaatatataactatatatgttatatatagtttatatataatattatatattatatataaactatatatatcacatatagtttatatattttttaatatataatatataactatatatttaaatatataaatatgtaactatatatttatatataactatatatttaaatataaatatatatttaaatataaattatataaatatacataaaaatacatatttaaatataatatataaatatatatttaaatataaatatatatttatatattatattataaatattataattatatattatatattataaatatataatatatattatatattaatataagtataaatatataaatatatattatgtaagtataaatatataaatatatattatataagtataaatatagaaatatatattatataagtataaatatagaaatatatatttaagtataaatatataaatatataaatatatatttaagtatatataaatatatataaatatatatttaagtataaatatatatttaagtatatataaatatatataaatatatatttaagtacaaatatataaatatatataaatatatatttaagtataaatatagaaatatatatttaaatataagtatatatatatttaaatataagtatatacataaatatatatatttaaatataagtatatacataaatatatatatttaaatataaatatatatttcaatataagtatatataaatatatatttaaatataagtatatatataaatatatatttaaatataagtatatatataaatatatatttaaatataagtatatatataaatatatatttaaatataagtatatatataaatatatatatttaaatataagtatatatataaatatatatatttaaatataagtatatatataaatatatatatttaaatataagtatatatataaatatatatatttaaatataagtatatatataaatatatatatttaaatataagtatatatataaatatatatatttaaatataagtatatatataaatatatatatttaaatataagtatatatataaatatatatatttaaatataagtatatatataaatatatataaatataagtatatatataaatatatatatttaaataaatatatatatattttttgagacggagtctcgctcaggctggagtgcagtggcatgatcatggctcactgcaacctctgcctcctgagttcaagcaattctcctgcctcagcctcccaagtagctgagattacaggcatgtgccaccggaccctgctaatttttgtatttttggtagagtcagagtttcgccaagttggccaggccagtctcaaactcctaaccttgagtaatctgcccactttggcctcccaaagttctgggattacaggcatgagcctgtttgtgttaactctttatcagcgtagccaatcgatgactcactcttggccttgtcacccactggaagtagtctgcatttaaaatcttcagcttcagtatgtcactctcatcatggacattttttggtacagccttgtcagatgagcaaaacagtttcccatactcatgatttgcctccaagtagagtttgtgggcacaaacttccattgtctccttctcagctaacaacagtgtctcagtctctctcattctcctactcttgctatacaacctcaacatcaccagcatcatcatgttctcttaccttgattcgtctctgttctcctttccattgattccccaatggggcttttatttccttccatacttagttctttcttctacctggtgatgtatcatcaaacactgccttaccagcatctttaaatctcatggcctcctgtcctgtaagcatctgctacttgcaaactgtcaatcttacaatgatgtaaccttcatcttctcttctctgacatgcttataaccatacatacgggcaccacacaaattagaggaatttaggtctacttgggacatttatttttagtttttgtgttgttgtctttcctcattatttcttatcttttgttattttgctggaggaaatcattaggtacttttttcagagtgtgaagatgtaaaatatttcctgaatttgtgcacgattggaagtcttcattttgctcgcattgttggataataattttgctgcatgcagaatgagagttttaaaatcccttttctttagttttagtagatatcctctgttgcagatgagaagtctttttcttatttctttacaagtaaatctgatgtctctcttaaaaagtttaggatttttcttttttttctagagttcataaatttcacaaattgacaagaatatattaggtgtatttttaagaattattgctactcagttttaggtgggccttgtttttttgtttgtttggttggttggtttttgttttgttttggtttttttgagacaagatctcactctgtaactcaggctgtagtgcagttgtgtgaccatggctcactgcagccttgactgcttgggctaaagcaatcctcctgcttcagcctcccaagtagctaggactacaggcatgagccaccataccaggctacttttatttatttatttacttttttttggtagagataagttctcagtgtgttgcacatactgagtcttgaagtcctgtgctcaagtgatcctcccaccttggccttccaaagtatcgagattataggagtgagccactgcacccagcctaggtgggcctttttgatctaaagaccaaagctttttagaaaggtacattttctctattttttttcttttttctttctctatgacttcaaatttttcttctcatattttctgcatgtcttaatattctactctcttgaagattccttttactttttcttcatattatgaatgtggtctccagtatgactattctgtcattctgagtgttttggtcttaaaaactatatttttaatttctaagaatttccttgatgtctctgttgtcataacaaactcacttgtatgatacaatattcacttaagcttctaaggatatcactagtgcatcttttaaaaagacattttcttttttcccctgcattacttttatttccttcagaaaaaaaaaaattgattttgtttgggttccatccttttatgtttacaatttttctcaaattgatcttttattttttgatcataataaatatacatggagtgttcagttaattaggataaaaagctggtgggaatagcatgactttatggaatactgatttggtacttactgtgagtaagtaggtggggatctgcctatatgcaggcttcccttcagtatgcagagaagtaagaaatcaaataaggaagttggaatgctctcatttgctaaataatgaagacttcattctgaggatgaaacacattcaagtatttccttactttgtgacactacctttcttttgaatatttttgcatattctctctggcccaattgttcttgatttgggttctatactaaatccccttttcttttcttgccttcttttttccaacctctcctttggagatctcatccattcatgtggatatacaccggtgtggccattccaattgctaaaattttggaatgcttccatacttcttggaaagatccgttttataaggctcccgagatcccccaacccctatcactactccagctctcttaagccctcccccaagagctccctcccctaaattcctaatggtccatcagctaaagtctcaatgctggcacagcgggatacttccagcatcctgctttaaggtgaccagttctgattggtcagtacttatgactccaccctgaatcatgttcccttacagtggggatccatggctatgcgtcaataccctagtcattcctagttgactttacattcaatttcccaaaagggaattcctcacccctcacctaacagatgaccaagacatcaagtcactgagaagataggagatttttgctttaagcaaaggtctttacctaagacattatctgtgcctcagctttaccatagagatcttgttcagggaacctatggaaggcatagataacactttatttgactgtcataggaatatcaagtgctcagctaagtttttcccagcaactgtatgacaatacaactcacaattgaggaacctggccagtactgtatttacatgtaggtatttacagtttattagaaggagagttgtatgctatagatagcaggcaaagtaataaagttgttattttcctatattttaaagcctttcttgtatttttgctataacacagggccgcaggaactctaaatgacagtgaatgcttagattaaaaaccaacaggtctcacgcctgtaatcccggcactttgggaggccgaggcgggcagaacacgaggtcaggagatcaagatcatcctgacaacagggtgaaaccccgtctccactaaaaatacaaaaaaaaaaaaaaaaaaaaattagccgggtgtggtggtgggcgcctgtagtcccagctactcgggaagctgaggcaggacaatggcatgaacccgggaggcggaacttgcagtgggccgagatcgcgccactgcactccagcctgggcgacagagcgagactccgtctcaaaaaaaaaaaaaaaaaaaaacaaaaaaacaaaacccaacaggtcatgaaggctttgcaagtatctcttaccaccaacaacaccaaagcccccattcctctccctcacattgaagtagaagaaaacaaagtccatatttcatgcgttcctttgtcaaaataacaaattggttacggcacatgtcctgcaagaaataaaatgtgtccattgctcagtagtctctcttgcgaagtccccctctgtatctgtaacgtaaaattcctaaggacataaatgtagttttaactattttacgaatagtactaaaataactggcatcctgaacttatcaattcttttgtatattagatttaataactttctttgaaagtttcatatttgagaataaaaattaactggcataaactatttcttctgaaagattaaaagtgtttgaacagtaaattacatgtttgagtccccccaaattgtatgacagaatttcttctgattgttaatataataatatctggtttactttctagagaatgaatgtatttaattaataatcataaaatttaatagtcaaattaagatggaagctaaagggaaaataggtcataatttctaactaggtaagaatatagttatatcaaaattttcttttcaaatgttttatgtctatcagtatgaattaaataatgttgaattgctatggtgacagtctatgttaatcagcttttattaactatcttttaaaatgaagaatgctatggtaacttcacactgttaatatttttcataaatttgtgttttgctagtaagattctaataaagcagagacaaccaagtgtttctaaactgatttctgtttgagatctactgaggaaaactgtctttgcatttggtgaattttgtaatgtaacatgcctcaaatgattctcaaaacagatgtttagaattaaattgaaagttagaaactctaggtttaataaagccatattgccttatttattttgaattttcatttctttttagaacctaaaataactcgtcctcccataaatgtgaaaataatagagggattaaaagcagtcctaccatgtactacaatgggtaatcccaaaccatcagtgtcttggataaagggagacagccctctcagggtaagtggttatgatgttaaaacacatatataaaatgtattttaaaatataacatttctttacacactcagttacacacttacaaacttctagtataaagtagtttccatgtacattttaaccaaatgtaaatatttctgtttgcatatatgcaaactgcatggcatttcatttgggtctggctagggaaaaaagagatgggaaccaataatgttaaagatagaaaaggaggcaatgaaaaagcaaacctggaatcctttgtatttgttggctggctaaatcatttatttttttttaatgttgaatctgagacttaggggatgaaaaagaaaaactaaaacccaggctttagaactgacagtttctggaaggagggagggagacaaggttcggaagaaaagaaatgagatctctggagagaagttaggcgaaaagactcaagactcatacacaaataccttggcaaatccctcctgagtcatgaagagtagtcacgcctgtaattccagcacttcaggatgccaaggcctgaggtcagaagttcaagaccagcctggccaacatggcgaaatcctgtctctactaaaaatacaaaaattagctgggtgtggtggtgtgtgcctataatcccagctactcaggaggctgaggcaggagaataacttgaacccgagaggcagaggttgcaagtgagccaagatctcaccactgcactccagcctgggcaacagaatgagactccatctcaaaaaaaaaaaaagttattatccatatgcttattttgtactgcctctgtatcttatccccacatttattgataaattattatgaggcatcattattgtctttccatctttatatcacctaaaagattacagactatgtttttcatttaacaatgcatagtatattttaagaggaatacaaaggtgtcactttatgtcaattaactgaactaaggtataaaattgctcagctttgctttatttaaaataaaataaatattgctaatttttaatgattaattatctggctttcctcttgttataaatcttggtgcatttcctacaatgacaaacaagggtggttgtacttatgttactgaatttagggttcaaaacagaaaagacagatgtaatatttaaactccaaagagtaattttaatattatgtgtgaaattttctagtatttcaaaggacatgtactcttctaaagataccatctagggtgcaaggtaaacctgatagttaaaagcaatgtgtttatttattcatatttcaatagcttttggggtactagtggtttttggttgcaaggatgaactgtatagtggccaagtctgagattttagcgtgcctattacctgagtgctgtacattatacccagtatatagttttgtatgtctcaccaccccactctgcaacatttcccccttctaaaagcaatgcttttaatcagatttagctgtgactttggggaagcaaaataagcccatatacatatatatattatacatatacatattatatatataatatatatatacacacactcatgggatgaaagaaaataaaaataaactatacctcataggaccctggtgaagattaatttaggtactcttgcactatttcatttaatattgtatgttttacataataagtggccaataaaggtcaaagcgatatctttgatgatgataatagtgatgacaataagttgatgatatttggcaaatgtatccttgatagacccataaacatttttgaattcacgtccctatctctggcaggaaaattcccgaattgcagttcttgaatctgggagcttgaggattcataacgtacaaaaggaagatgcaggacagtatcgatgtgtggcaaaaaacagcctcgggacagcatattccaaagtggtgaagctggaagttgagggtaaggagctgcatttcttcccctgactgtgtgaccaggggcctcactgtctactgtgaaggctgcacctgggcttgggagagaagagatgtgggcagcccacttccctcagttgttccagatattaggccataagttcaagcgtatgataacagaaaaggaaaactgaaaaaagaaagaaattttcactagtataaaaggtcatattccattgattactcacaatcactggtaatttttaccaaaacagactaaatttactgtaacagtaaaataatagaaacccattttagaatgccttcctggtttggacaattcttccctcatttaaagacttgggcataataattttaatctttttaatcatacagaacaactttaaaaagatgtgataaggaggaaaaagtagctattttcagatagtgttctatcacaaagcttgaaaaaaaaaacattaactcaaatcgtaaagccaaaaataatcccagttctgtgagttatgttatcaaaatcgtttttcttttctttctgaaaatattttttaatcagctaattcaataacttggtgtgaaaggtcttcactgagaaatcagaatccaaataggtctaggtgttctatttgcttgttagtcgggtccttcttgacctaaaattgcaccacattttgcttaaaaagaactatcagcacacctctattaggcgaataaactgagtacaaacccttcctagctcttcctgttgtatgccagagaattaacatttttggctcctagctttctcacctgttaagtggggtttgctctatttaatgaagttaggtgttgatagtgtttagcagagcatctggtcctttgtgcaccctcaataaatgttggctataagaattatcatctatttttctgcttttctcttatctgattgataagtgttagagtatcacatactggcacaaattacagttcaatcaggaaaagacttgaagagttctccatattttactttgattctgtcctactttccttaggccaaaatctatctttttctgcatatttccttctcattcatatagttttctattaactcctttaagattatttttatttccttgatggacattcctgtcacagctttaatgatctcgtttcccacgtttgcctccaattgaggtaacatctttattatgtcattgtaccaattgctcataaaattttcattaattcctcatggaaaataatgaggttggtgggaatagtggctgtcagaatataaaaatacattttcatcaagttgctgtaatttaatatcttctaaataatcagtgctatttgaagctccgtattcaacaaccagttgatatgatttattcagtaaaaactaaataaacctacaaacccacccagacacacattcagactcactgaatatctgtctgcctagatccagtgttttcttgttctgtctaaggaatgatgcagaattaatgctatccatttgtataaaatatcacaaagagtttttctctttaagatattaaaaataagctttctgtattataatatttaatactgatcatttaggtcactagcttcatagatattaaaacaacttgctttacctttgacctagagaaaattatcaccgaactccattagttggagttattatacagtttggttttgtgttaccatttaattttgtatcatgcctgtcagtaacattgaatactgcctcttttctcgtaacctttaaatggtcatgcttagtgctttaaaagttaatatatgctagagatagtattaaatttgctagaactgagagctcataatatcatttaaagttattctaactgtaggtaagatgattttcgcacacatttagcacacatgcctatttctaccctcatcttagtccctattaaaatagccaaaatatgtgacatgcttagcacaattgctggtacataataagcatttaataaatggaatctactatacttattatgattaaaacataaaattgtaacaaatttgctttggctctggaagctctgaaaggttgacttccacttgaataaatagttgaggtattttcataagatataatgtagacgtgacctatttagaggtaaggctaactcaacctacaactcagaacaagtttttttttaaaaaaaagcaactgagaaatgactggaaaaacctcaaaagtacatgcagatgcctgctagtttggcaatgatgaggactgaagatgatgaggcagggccatcgtcagttggggcagacgagcatatgtatgtatcaagtcgtaactctgcaaattagttatgcagtgactatatcgaatcagaactctatggtgccatttgatgctgggctacatgtaacacagatgggcatacaggaaaagatagagttttgtcattgatagcctctaataaagataaacatgccaggccatggattgggtaccatgtgttaactaaatctttgttgttacaactgtgaaagaaacaccctaccacacagaagtaaagctccctttccagattgccattctcagactgctggacctctgcaatggaaatgtaaaggtatcctatactttggtcttgccttaaaatgttcaaacggtgacatacataattattgatggaaccatctaactcaagcgtggtgcagcatggatgtctttctgaaaggatgtccaatgggaaggctaacattttaaaaagttccattgaggctaaatgttttatgtggattatcttgtttaattctcactaaaagtctgtaacaagggccctatattctgccattttaggaaacagaggaatagagaggatagctaacttacacaaggctgcacagatagctgtgtctaagaatttgagtcaggctgaatactgtctgttctattaaagaatgagatagtcctgtgtgacattgtcaagagaagtatctagatgaaaatgaggggaaaaccagaattctgcaaatgaaggagaggatgggagctgaaaaaaaatagacaggaggtattgacaaattttttgataaatttttcagagaaaagggaggagtcaaaaaaggaagcagtagctggagctatgtttggtgtgtagatcacgttgcattgcaagatttctcttcgaaaaatattgaaatgtaaaaaaatttaaatgttaaaatattaatgacactgagctcagagagaagaaaaatttgaacatacataagagaacaaaatactcaatgaaatgtggtgctggataatgcacaagggttgtatttgggggccaagtagaatgataaactgtatacagagggtgaccacatgttctggttttcccaggacagttctggtttacacctgttgtcctggaatcattattaatagagtctctcttttactttcagaagtatccaagcttccacgataaattatatggttactctccttagacaggaagacagaatgtccttgtaataggaagaaaagagaagagggaagttttcagtataaatcagttcataagtatgatggatggaatttgagaaaattctgttcttatggtctctatttcctctaggaaggagaaattagggttatcttttgaaaacacttttgttaaagggtcagggaagggggtgaggatgaagtgagttctaaaatagctgtggaatgggagagatgactcacaaagtaaaccaggattgtagggcagcattaaagttttagtagagagcaggaccgtgaatatggagtagcatcaatccacatggttgcagggtttttccctccagtgttgctctgctgtacaggtgtaagttcagaaaatagcagaaagctaaattcaaaggcaagacataaatgttggagttttgtcaggaaggaaaatagggcaaaggatttgagactattgcaaaagagagattgaagcatgaatcatggaatctttaggtaccaaatacacctataatttctctttcttttcttttctttctttccgtctttcttgtttgtttgttttgttttgtgacaaagtctcactctgttgctcaggctggagtgcaatggcacaatcatagcttactatagcctcaaactcctaggcttaagtgattctcccacctcagcttcttgattagctggggctacaggtgtgcactactgtgctcagctatttattttatttattttattttattttttttactttactttattttattttattttattttattttattttattttattttattttattttattttattttattttattttattttattttattttattttattttattttattttattttattttattttattttattttattttattttattttattttattttattttattttattttattttattttattttattttattttattttattttattttattttattttattttattttatttcatagagacagggtcttgctatgttgcccaggctggtctcaaactcctcaagcaatcctccagccttgacctcccaaaatgttgggattataggcatgagctaccatgcccagccatacctatgatttaatatccaaatatgaaatatcctcggatatgagcctaggattaaacactatagctccagcaatataacagacaagataatatgaaaatcttccaactagcccggaaatgctgggctagttgtataactgataagaataaaatataaatctctggaagctcaaaatggacaggaactgcaaaaaccataagctcaatgagctggtttcttattgttctaaaggtaatgggacaggtgctcatctccataacctaggagattggttttaagacccttaaaggggcaggagacagggtcttggcccatgtgaaatggaggttagcattcaaaaccttgtataaagctgagaaccacaaaagtagaaagatgggccagaaattccatcttcaggcacaaaaacacaacacaatggcttgtctgtctttagtcgtgagggagaaaaaaccaggtcatcttaggagtgaatattcaacactcaggactatagcttccataggcttgatattgacttaataccatcagcacagtctgggaattctcagctggaaaaatgaacatacaaactggtttcaggctgggcacagtggctcacccctgtaatcccagcacattgggaggctgatggcttgagcactggagaccaacctgggcgacatggcaaaactccatctctacaaaaaatataaaaattagccaggtgtggtggtacatgcctctagtctcaactatttgggagcctgaagtgggaggatggcttgaacctgggaggtggaggctgcagtaagccaagattgtgccactgcacttcagcctgggcaacagagtgagaccctgtctcaaaataaaaaaataataataaaaaacctacatacacataaaacattggttccaggccaaaaaacccacacacacacaaaacattggttccaggccaaaaatttaacaagaaaaaaaagctaaaagaagaagcactttacccaagttacataggcttttcatggatatacctacactgaggatgagttcacaatgcaaaattgtcacacctacagaaaacaacctaccttgagtgagtgttgtccaacataatatacaaagaaaattcagttaaccacataacatcatagaaattataaaataagtatgcttaaagtaattaaaaatgcaaaaggaggaataaggaaaacaaaaatacattagggccaggcacagtgactcacgcctgtaatcccagaactttgggaggccaaggcaggcagattacttgaggtcaggagttcaagaccagcctggccaacattgtgaaaccctgtctctatacaaaaattagccgggcgtggtggtgcacgcctgtagttccagctactcagaaggctgagacaaaaggatctcttaaacccaggaggcagaggttgcagtgagccaagattgcaccactacactccagcctgggcaacagagtgagactttgtctaaaaaaaaaaattaaataaataaataaattaggaagacaggcggttttgataaataattaaacacaattttaacacataaaaatataattaaaattaaaattcaatggatgagtaatagtatattagagataaagagataattcagccattagaaatcagctcttcagaatttagaattcagtgcagaaagattttgaaagcatagaaaagatgaaattgaatttaagaaatgataaaagatacattacagaaagagaaaatacagaaaatatgagaggcaatactcaaaaacatgatgactaagaactttccagcattgatgaaagacttaaatcttcaagtacgtgaagcataccaagtcccaatcggtaaaaatgaagagcagagaaataagcatttcacacccagtcaaatcataaggaaacatcaaagacaaagagattatcttaaaatattgaagacagattatttataagggacaacaattaggataacaatagagtgtggggaaactatcactttcatctatccttacaatttggtatagccgcatgaaggtttatgaaaaataaaaatcaaactttaaaatgtatatagatttgtctcagcaatttaacttctgacaatttatctaaaagaatatttgcacaagtgtatgttcaagattatttgttacaaagttacttagtaaaaagttgtaaagaacttaaatttgcttcaataggacaccatctaaatatgtgatgtaattccatgcagccattaaaaagaatgagatggatttatatacattaacttctaaaattatctaccgtatatgtgacttgggtctgctttccatctctgagcctcagcttcctcttctctaaaatagagataaaagcagaacctacctcttaggatgcttgattattagttattatagacacatagtgtgtactatataattatttgctgttttattatttatttcatgtatttaataagtacttgtatagtgcttaccatgtgcaaggtactgttctaagtgctttagaatattaattcattatcaatataatatgttcataagtgaaaaagtaagtaaagaacagtatatcttattattatatattagagtacatgtatatcgaactcttaagtcatcatctctgcatagagaattatatggggggtcccggcacgatggctcacgcctataatcccagcactttgggaggccgaggtgggcgaatcacgaggttaggagtttgagaccagcctggccaacatggtgaaaccccatctctactaaaaatacaaaaaattatctgggcatggtggcaggcacctgaaattctagctactcaggaggctgaggcaggaaaatcacttgaacccaggaggcagaggttgcagtgagcagagattgtgccactgcactccagcccaggcaacagtgcaagattccatctcaaaaaaaaaaaaaagaaagaaagaaaattatatggggggtaacttctattttatactttatgtaggcctatactttttgcatctttataaagagaactttataattaggaagaattaatacatgtatttatgctttggaaaaaaaacacttcttccaaatattatactatttttgataaagtaacatgaatctttctgttcattagtttgatatttttgcagttctgttgctccattgatctccaagacaaactcagggatgggaaaagttttgtcaaaggtaattgattcctatgttgcttaggttttcacaactcttggatattggctgaagaattttgaaggtggaggttctgatgaacaggacatagaacatgccatttctttagggaaggaatcaatccataaagctgctaagcttgagacctacaagaggaagctggcagagtcacctagatggaggaagtgaagggggtcagagaacagcagctgctgctgaagtctccactcatctgactcttcttttctatgtatagatggaaatctgaaatttatgattttatagtctggaatagtttgcaggatccagcacaaaagttttgttagggttctgatttctcagtgaacattttccacactttgttgggtgttactgacttacagcataaagggaaaagtaaaacaaagggaagttcctataggaaagggaagcaccgctaggtaagaactatttaggtatactgatttgacattgacagggaagcctcagtcatctgggaggcagaagaaagttttcagtctcacaactgtggaggctcttctggaaaggttaagccctgtctcaggcccccatgtgccaaagggctctaggagaggattcacggcaggatgtgaccagggaacatcttattgtctgatgtcaccagagaaatctacatatcatatttcatacattctaagatgctcattatttcatattttggcatatctgggttacatctcatcattaatcccttacaatcacaattgaataaattttagtttctttgttgcacattttaatatctctgaaattgagatttttcttacaggtgctaaaggcattcacacccagtttgtggcttctgccctccagctaagatcgttccttagctgtccccaaatatgagaatgaatatacaatgataaaacaaaaataaatcaaccatattggctcataaagctttctttatttttaactagaaattttccttctgcttgagatcatttcatttgttcctcacagaaatgaaagcattattttcagtgagatgatgccacctagcctttttccctcaaaaccttttatttgctgcctattcattttattaggttgaaatagagactaagtggcttttcagggccatatagaggaaattatcttttaagagacaattttgtctgcttctctaatccacttcacattctactttaggtatggtgtgtagccaaagagtgatctatgttgtttttcttatatatctgaaaccattttctcctgagtacctctaaaggacaggaagacagatactcaggggcagagggatatggcagtggaacatgctgatttgaatggcagggctggaattttcagtgagggtttgctatcatgattgcttgggacaacttttgaaattctgattttatttccctgttttgaccagtggcatatgccccctttaaaacgctagaccagagggccaaccatagtggctcacacctgtcatctcggcactttgggaggtcgaggcaggcggatcacttgaggtcaggagttagagaccagcctgggcaacatggtgaagccacgtctctactaaaaatacaaaaattagccgggcatggtggtgcacacctgtaatcccagctactcaggaggctgaggcaggagaactgcttgagcctgggaggtggaggttgcagtgagctgagatggagccactgaactccagcctgggcaacagattgagaccctgtctcaaaaaaatagagagagagagagagaaacaaataaagagagagagagagagagaattaactcctctgaccagtgcaggggctcatgcctgtaatcccaatacattggaaggccaaggtaggtgaacctcttgaatccaggagttcgagacaagcctgggtgacatggcaaaaacctcatctctacactgaagtaaaataaagtaaaataaaaataaaataccagaccatacaaatcaaatcttttaaggtgataacataattactgttttttatagctggggggtgtctttgttataagattttttaaacattgcaaaagactcatttaaaatttataaatatctaattttcttttttcccctcacattctctgccttaactaagattttttaaaatgcacttatttatttgaggctgaatgtggctcttactacattctgtgaatatcacattcagatgaatactgctttgtatatagaaactggctgaatgtagtgtacttgagacccaaattaaaatagatggatgctggagtgtatcaagctcttgttaattgcattttctattattatcttgtttacccttgaagctttaagccattaaggaaaatattgggcagaagctggaatcaagagagggctgtcagtcagcaattgctggtgggaagaggtgtcttggcaccaatacaaaactgcaaataagagccagggagggtctagaagcagaacaaaaagaagagagtccttctgggtacagggttattgtagggctccttcatgtttaaacaactccttatttcctcagggctttgaaaagcagcctcttgtcaaaacgtggagaggcattttggggagtagcaagggaaggcaagtcatatggagaatttaagtggcttcagtgcatgagtctccatcacttgtcaggacacctgcgtgctgccagcccagtttctttgcaatctgaaaggctgcactatctacccttaaggccaacaaatctattcagaaactttttatctgaagatatttttgcagacatatgaaataggtaagacaagtagaattccaaaagatgaaaataaataattcctatctatctatctatctatctatctatctatctatctatctatctatcatgtcacagaagcagatgtcaatagacataactataaaagatcagcattgtcttcaaaattttagacttctggctgccttctttcctagagatcaaatttacttgttgcatctacaaaagtggaagcaatcatgtaatgtttataccacatttgatttgctttttcctttgtttatgctgcagctcttaatatcacaaatgccacagagagaggtatttaaaaccccaccgtgtgctgtgctctcagtgattgcagttttaataaaatttgcagtggtcctgttcccggcatcagtcctaattgtttgatgccactaacaggtttgatttgctgtcatgagtacattgtgcatttgaaaaccaaacaaagaaaaaaatcacaaacctctgatatccctggaggctatcattgggaaaattaaatctgcatgatttaataggatgcatgttctaaaagaggagcttgacccattgattttgactttaggggatattatggtatattatggtatggtttcatctaaaaagttctataaggctttcagagaagaatggtaaagtggtcctttagttgaacagttgtcactgcatttctctaagaaaaagtcacttgtataataatgatagcactgaatagataatacataatgcttcaagtcaacacatagttaggccctcttaaagtgcagtgttaaagaaaatgtactttaaagtaattaaattgtttagtataatgttgacattatattatttgaaatgaaaagttatcgtgacctgaaatacatatactctaattggagtgagtttttaatatcagttttcagatattaaaatgagatgcataagttcaagcatacatacacagtaaaggaaaattgctttctattcaatgcattagttcatccaacaaagacacactgagcaatgttccaggtgcgacatgagacagagaaggccatgcctccactgggcttaatttcaaaatgtgactattttgattcatgcaaagatatcatggaaagctattagaattctatggcaagaaggagttcaagtaattttatattcagttattttcttcagatatcaaatacatgactctgagactgtaattgttatatttatttccgtgaacaacaaactatacaaggcattatttttggagaggacttaatatagtggttaataacatgggtggtaggatctcaagaaaactgcatgggctctagagtctcaggtaaacttcttgagtccaaatcctggttctaccacctatcacatgtgtaacttccagtaggttacttatctctttgtgcttcagtttccctacttgtaaaattaggttaataatactatctacttcataaattgaagaatgtagaagtagatgatatatttatagctcttacaagagtgactgacacagagtaaacactcaaatagaaattatcaatgttagttctagggctaatataacttctgtgcaacagaagcagtgatttacgcacttgtgcagacaattatagctcccttttccatgtgtcatctttatctctgaggtacaagcaataaagtccaatggcaaaaactaaatttttatttttaatagtttatttgtattaattttggttctagcaaaagaataataaccaactttaaatctgcaaggagaccccaaagtcatatagttcaattccttagtttgtagataaagaaatttgagactaggggcagtggctcacgcctgtaatcccagccctttgggaggctgaggcaggaggattgcttgaggccaggagttcgaggccagcctgggaaacatagtgagacccccctctctaaaaaaagtatttatttaaaaatatttaaaaaaaataaatttgagaccggaaacatcttttattttatagataaagacacttgagaccaaacaaattgtgactttgcccaggttggcaaaggaacttgagtgtcttattcctagtctaggacatgttccactacctattccgattctttcctctttttaatatttatgttgtgtagataatactctgcagagttcacatgctttagtatcagacagacctgggtgttaatcctgactgtgctgcttaccagttatgtgaggtaggataatttacttgacctctgcaagtctatttcctcatttgtgaaatggggggtaatattatctagaacacagagttcttaaaatggactcagatagcatattcacagtgtctatgcagtagctctttgataagtatttgctgttgttggtattgttgtctgttgtttacattgttataaagctggatgacatctatccagagaataccactagtcaagcttcttgaatttcataaatagaatgtcaacattaacaactgctcatctggtctccaaagaacataaatctactttcaatcagcaatgctttgtacctgcattcttgttacttgttagagattataactttggtgtggcttcaccgtgacaagtgataaagggaagcttatctaagaagctgtgtaaagaggtgcctttaatggctaacaggagagaggctatttggagtcaacctgttaaaaaaaaaaatagtacaacaacataaaagtgaaggcggccttcgtgtgtatcctgagcttatatcatattttattcactatagaaatagatatttgccaaaaatcttgttgaaaatactgatccattctagcactcactgctcccaagtaaatcccaagtgagaaatcactttaaaaatttagtgccataaaaaatgatgagttcatgtcctttgtagggacatagatgaagctggaaaccatcattctcagcaaactattgcaaggacaaaaaaccaaacaccgcatgttctcactcataggtgggaattgaacaatgagaacacttggacacaggaagggggacatcacacaccggagcctgttgtggggtcgggggagaggggagggatagatagcattaggagatatacctaatgtaaatgatgagttaatgggtgcagcacaccaacatggcacatgtatacatatgtaacaaacctgcacgttgtgcacaggtaccctagaacttaaagtataataataataaaaaaaatttagtgcctggctgaagctctgtgaacaatggctgcagaggagcttcctgccagcctggggaagccatagcctgcacatgatctgtcccttttagtgaactaaggttgacttgatggagccaatgctggcaaaggtctctgaggaaaactgacctggatttatacaaggtccagccttgcaggttgtaagggaggtcactttcatggcaggccaagatccttgagatgttttgaaaatttgaaggagaaaggaattaggccaaaatgaatagatactgcagacgaaatcttatggagaaatttccttggacttacatccttagcctcagcacagcaaattaatagaagcttgcagaagaccaagctgagatttcttatagagacctccagtggaacaaacttaaagaaggcccatatggttaacgttcttgatgcccatatggttaacattgttgatgcacctgtgtaaagaaccaggctaaatctaataaggccatactcactttgtcgtcaagaatctttgagattctttttaatcaaaagtggggagactataaggaacaatttgtgcttaaatggaaaattgtgtgttgtcattctagagcacacctgtgttgtgtgattccaattctgtgggagctgttttaaagctttataaattgtagataactggcagcaatgctaaatcgttctcctccatagacttgcaaatgaattctgtccagtggaaaagcaaccaaaaaacccaatctggctctgtttgcacagtcattttaatattcctgatctataaatgggtctgtccttgggattctcttttcacctggttgcaacacttcactggtttccttgtgaatgagttaaataaaacctttaactcatgtttattctgaaaaaaaaaaaaaaaatcagtgcctgggttggggagaagggtctctgctgagaaactgacattttatccattcctatagtaagtactgtgtactttattttcttttactttattttttgctcggcctggcatgtatcttagaatccaatttgaagcttagtcatagtatctgttacaccaaaccatccagaggttttctccctgatcctaatatattgttattcatttaatttatggccatatcaatataccttccttaagatctctatggaaagagaaagaaataactaagttgacggattcatggaacttctcctccaagtggttctcaaatgccagtgcacatcagaagcatctggagatcttactaaatgacacagcttcaggatgcattccccagttattcatatttgataggtttatgttagtgctttttaacaacaccccaaaggatccttaatgcagtgacccatgaaccacactttgaaaaacactgacttcaggttttctacttagagggactttattccagatgaatctaaacaattcaaggagaaagcttatctgcaaatagtaagatatcaatgttaggatagcttcaaattcatccatttgtggaatgcctactatgttctatgcaatgaggtaattcttaaggatacagacgaaagagagatgtggttcatttcttgaaaagtgttcaaattcagtgaggaagatggatatgcaaacaagtaaaatgcaatatatttacaatgatagaagtttgtgtagggttatatacacaggacagaggagatagggaaaggtcagggaaatcttcagagaggtgaaggccaacagggctttgattgatgaagagtgaggagctcttggtgaggatcatatcacattgagggaggaaaaagaaaatagaggaaaaaattcagatttaccatgcaagcacagcctgacactgactcataaccagggagtaaaagaaggattagagagtttgggacagtatctattagtggtgctataaaaacagaataaaccatgatttgactcacaggctaacatggagatcatcttcttggtatcttctcttggacctttttttctttttttttttttttgagacagagtctctctctgtcattcaggctgaagtgcagtggtgcaatctctgctcactgcaacctccgcctcccaggttcaagcgattcttatgcttcagcctcctgagtagctaggattacaggcatgtgccaccatgcccagctaattcttgtatttttagtggagatggggcttcaccatgttggccagtctgctctcaagctcctggcctcaagtgatccacctgcctcagtcttccaaagtgctaggattacaggtgtgagccaccatgcctagccctctcttagacataattttaaacgggtagatacctaacatcctgacaatgtgggaacctatggtttggaggcatatggccttatctggcatccagaattccatctaaaaggttacagaaatgttcccaacccaagccactttaggcaactgggatactgtggtatgatttaagattaagtttatggcttctatttatttttccaactaaaatttccggttggctaaaacaaaaatcttattcttaatagacctcagctcgtttaacgcatgtcagagtgcaggcatcttccagagcttagatcaaaaaagttatttaaaagtaaactaagattttgataaacgtaccatggttatacaggatgctaacattaggggaagttgggtgaacagtacatggaaaccttctatactatctttgcaactcttatgtaaatctaaaattatttcaaaataaaaatgttttaaaaaggtaaactaagaaatacttctttgtcctggtaattaagcagtttagcatctcctcccatcaaaaacagaaaatgaactttctttaagaagaaaaaagtgaatcaggaatgaagtcacatattttattggctgatgattaaacttctaatatattttcagaaccagtttaacaaaagtgcggttgctttatatacacctaagactgaaacaaaaacaagtaaactttgtgtagctcttgtctgatttacatggtaaacatttcttgctcgtacccactattcaccatccatatttacaagtgagaaaaacgttaccatctcaaaggccacagaaatgttcccaacccaagccatgggagggaatcccacatcattgaggcaggcagcccttattttgtcctggagaagagagtcactgtatttgaggatgatgtgtatgttttctaaaggagcttgccttctgactccttcagagttggagccacagtgggtaaacctcttcaggcattgccctgcttcaaagcagacagaaacgtaaacagtggcagtacaccaggatcgggtttctttgctttagctaccaagagtcaggaaagagaaaccaggtatttatttaggtttgtcattattgctaggttttcttttaatgataagggtttgagaaaagaagtatctgttcagaaacaaagtatcacagagcctcaataggattactttttcaaccacccagggccacgtgttttttaagtcaaatataaaaacaatttaacccattttgttctttgtagtccttgccttctaacaggcatctccaactcagttattcttaaaatatgatgctagatgagacaaatagtctatttaagagcagattcaaataatctgtatagatgagcatctatgttttaataacagggtcgaaagaatttctctctctacagagtgatttctctgtctcaaataattcagtttatgaaaggagttaaggatatgctttttaacaccggaaaagggtacttaacatgatcaatatctaggatacagccagctaaagcagattgacgttacaaaataaaataaagccttctccctttgaagtccgctaaaggaacagctttcatgactcagaaagatatcctagagatcaaaaaagctctggagaaagaaaaagaactagaagacctggcttcaaaatttacctctagtacttcccagttatgagttagctgctatattaattaattgctttatataaatcatgtcatttaatcttctccacaattctatgagttgaaagttaataactacgttttctggataaaaaacctgaagttcagagaagttgaacagcaactcagaaaaaacagctagtgagagtgaagtgaggatccaattctaccacctggattgctgttagggtattgtacatgagtgtaaggaaagtatcctactactaagattttgacttcccaacctctgagtttgtcctttgaaaaagccctgcaatggtgttcttatttaccaaatggtcagagattttctcaagatattgactataaggaaaatgataaacaacttaatgaaatggcactatgaaaataaaataatctaggaacaaatattattttattttcatagtgccatttcattaaattgcagcactattcacaatagcaaagacttggaaccaacctaaaagcccatcaatgatagactggataaagaaaatgtggtacatatatgccatggaatactatgcagccataaaaaggaaagagatcatgccctttgaagggacatagatggagctggaagccattatcctcagcaaactaacacaggaacacaaaaccaaacaccacatgttgtcacttataagtgggagctgaacgatgagaacacatggacacatggggggaacaatacacactggggcctgttggaggctgggagtgggaggaaggagagcttcaggaagagtagctaacggatgctaggcttattacctaagtgatgggctgatctgtgcagcaaaccaccgtggcacacgtttacctatgtaacaaacctgcacatcctgcacttgtacccctgaacttaaaatagaagttgggggaaaaaaaagaaaaaagaaaatattagcttggtgcaaaagaaataatggcaaaaactgcaattactttttttgcactaacctaataaagtgatcttcagaaaacagtctacttttttaaaaggcaaaaaacagagtggtttatataattggaataatttttacatctctttttgatacacacttcttattttctaaaattttcacaaatcatcttaaacattaatgctgtcaccaaataaattctcacaaagtaatagaaagctcaggaaattaatatttgagaagtaaatttagaggcaaggataacttaaactgagaagcatttataattttccattttgagaaatactctgtagcatttataaacattatattttaattttctatttcaataaattaatattgtaccagaataccaaactgcatttggaatcataggcaactgttatgtttgtcctttcttgatatttgaaaaaaatgaccttccttttgaatgctgccctcataatttaaaattggccttctccttcacaggggacattcacgataaactctcaataggctttaatttggagaattttagttttgccttagcagcttttcttatatcttctcttaattctataagtttaggctttatatgttttgaaattatattgttaagtgcatacacattaattattgttgtaattcctcctctgcagattgcatttttaacctttggatgatgttcctcttcatcccttttttataataccttttagcataattctgtattgtttgatattaatatcattaccacatttttcctttgttttgtgttttcctgtgctctttgtttggtattcttctatccctttcttccaacctccccacaatcttgtaagcagaatatagataaatacttttgtaattcaatccaaaagactttatttttagtagataagtgtaggtctttgtatttattctgaaaatttatttctattattctttagacgatttctatcatgtattatgctgtttcttttctctgcttctttttttaattccttttctgttttctggtagatcattatatatttttttaattctccactttaacccacaccacaaccactttgctgattttgaaattgtggatcacaatgttatatttcagtgattacttttacattttaatatacatatatgatgctataattctaacagtttaagatcatgtaggatcttctccagtctctttaactaaagcaagcatcttatcattctttaaaatcttatctaacagcaccttccctcatcctctgtcactttccttggcattatccaaaattttatttatcttaaaacactgaattattttatggtccataaatagttctatcaatatatgccaccaattgctgtgttcatcattcaattttcatgtgccttttttcttctgtttccaatttcttatttctgaaacatattattcattagttcattcaattagatctgtgagcagatatctttgtctttgtctttgtatgtttgaaaatgccactattttaccctgatcattcaggtgaacattgaaatctaagttgacagatattttccctcagtgttgtcctatgtcttcttacatcaattgatgagaagtgttctgtcattgtaactataattctttggtaggtaacagagttcttctatctggtctttttacctttgattaccagtacttttaccataatttgtcatagaatctgtattagttatctgtttctctgtaacgaattacacagaaactcaatgttttaaaacaataaacgattatcacttcatagtttctataactcaggaagtacagagtgactttcccaggccaatgtatggattatgtttgcagtaaagatgttggccacttctgcagtcatctgaaagcttgaatgggctgaaggatctaattcaaatgtggttgactcataagttggtataaattgatgctggccagtgcagagcctccctttgtccccatgtgggtctctcctcaggctgcttgagtgtccccacactatggtgccttccttccctaagagtagatgatctaatagagaacaaggaggagttagtaatgtttttcatggtctagcttcaaaagccacacacagtcacttctgccacatcctatttgttagaagtgagtctaagtccagcctacttccaaggagagcagaggaactatactctacttttccaagggagaatttgtggacaggttttaaaactacaatagtgtcaatgcaaaatgcaccagaaagtctcagtaacagaatcacacaagcagaagaaagaacttcagagcttgaagacaaggcttgaactaaccaaatccatcaaagacaaagaaaaaataatttttaaaaatggacaaagcctccaagaagtttaggactatgttaaaagtcaaaacctaagaatagttggaaggaagaagagaaagaagataaatctaaaagtctggaaaacatatttgaggaaataatcaaggaaaacttccctggcctcgctagagatctagacatccgaatacaagaagctcaaagaacacctgggaaattcattgcaaaaaaatcattgcctagacacatagttatcaggttatctagaatcaagacaaagaaaagaatcttgagagctgtgaggcaaaagcatcaggcatcttataaaggaaaatctatcagagtaacagcagatttctcagcagaaaccctacaagctagaagaaattggggtcctatttttagcctcctaaaacaaagcaattatcagccaagaattttgtatccagtaaaactaagcttcataaatgaaggaaagatacagtctttccagacaaatgctgaattagccaccacaaagccagcactacaatctcaaatgaactctaaatcttgaaacagattctggaaatacaccaaaatagaacctctttaaagtttacatctcacaggacctatataacagtaacacaatgaaaacaacaacaacaacaacaacaaggtattcaggcaacaaatagcacaatgaatagaatagtacctctcatctcaatactaacgtggaatgtaaatggcctaagtgctccacttaaaagatacagaatagcagaatggataagaatttcaccaaccaagtttctactgtcttcaagagactcacctaacacataaggactaacataaacttaaggtaaaggggtagaaaaacatattccatacaaacagacaccaaaagcgagcaggagtcgctattcttatatcagacaaaacaaactatagagcaatagcagttaagaaagacaaagagggagattatataatgataaaaacactagttcaacaggaaaatatcacaatcctaaatatatatacaccttaacactggagctcccaaatttataaaacaattattactagaccttaaaaataagatagatggcaacacactaacagtgagggactttaatactacactgatagcactagacagatcatcaggacagaaagtcaacaaataaacaatggaattaaactataccctacaacaaatggacctaacagatatttacagaacattctacccaacaaatgcagaatatacattgtaatcatcagcacatggaacattctccaagacagaccatatgataggccacaaaacaagtctcagtgaatttgagaaaatagaaattatatcaaatattctctcacaccacagtgaaataaaactggaaatcaactccaaaaggaaccctcaaaaccatgaaaatacatggaaattaaatgacctgctcctgaatgattgttgggtcaacaatgaagtcaagatggaaatttaaaaattatttggactgaacgataatagtaacaaatagtgagacaacctatcaaaacctctgggatacagcaaaaacggtactaagaggaaaattcatagcattatctgaaaaagcacatatagacaatccaaggccacacctccaattctccatgcaattggtgaaacaagaacaatctaaacacaaatgcagaagaagaaaagaaatataaaagatgagagcagaactaaatgaaattgaaatggaaaaaaatacaaaagataaatgaaacaaaaagctggttctttgaaaagataaataaaatcgatagaccattagctagattaaccaagaaaagaagacagaaaattcaaataagctttattagaaatgaaatggaaggtattacaactgatatcacagaaatatgaaacattattcaaggctaatatgaacacctttatggacataaactagaaaacctagaggagatggataaactcctggaagtatacaaccctcctagattaaaccaggaagatgtagaaactctgagcagaccaataacaagcagaaagattgaaatggtaattttaaaattgccaagaaaaagcagtccaggaccagacagattcatagctgaattgtatcagacattcaaagaagaattggtaccaatcctattgacactattccacaagatagacaaagagggaatcctccctaaattattctatgaaggcagtatcaccctaatactaaaccaggaaagagcataaccaaaaaagaaaactacagaccaatacacctgatgaacacagatgcaaaaattctcaacaaagtactagtgaactgaatccaacaacataccaaaaatataatctaccatgatcaagtgggtttcataccagggatgcagggatggtttaacatatgtaactcaataaatgtgatacaccacataaacagaatcaaaaacaaaaatcacgatcatctcagtagacacagaaaaagcatttgacaaaattaagcatcccattatgattaaaaccctcagcaaaatcagcatagaaaagacataccttaaggtaataaaagccatctatgacaaacccacagccaacgttatactgaatggggaaaagctgaaagtgttacccctgagaactgaaacaagacaaggatgcccactttcaccacttctattcaacatagtactggaagtcctagccagagcaatcagacaagagaaagaaataaagggcatccaaatcagtaaagaggaagtcagaatgtcactgtttgctgatgatataatcttatacctagaaaaccctaaagactcatccaaaaaactcttagaactggtagatgaattcagcaaagtttcaggatacaaaattaatgtacacaaatcagtagttctcctatacagcaacagtgaccaagctgaaaatcaaatcaagaactcaactgcttttacaatagctacaaaaataaaataaaatacttaagaatatatctaaccaaggaggtgaaagacctctacaaggaaaactacaaaatactgctgaaagaaatcatagatggcacaaagaaatggaaacacatcccgtgctcatagataggtagaatcaatattgtgaaaatgaccatactgcctaaagcaatctataattcaatgcaattcccatggaaataccacgattactctttacagaactagaaaaagcaatcctaaaattcatatggaacctaaaaagagcccacatggccaaagcaagactaagcaaaaagaacaaatctggatgcattaccttacccaacttcaaaccatactacaacgccacagacaccaaaacagcatggtactggtataaaaatgggcacagagatcagtggaactgaattgtgaacccagaaatgaagccaaatacttacagttaaccgatctttgacaaagcaaacaaaagtataaagtggggaaaggacaccctattcaataaatggtgctgggataattggcaagccacatgtagaagaatgaaactggatcctcacctctcaccttttacaaaaatcaactcaagatagatcaaggacttaaattgaagacctggaaccataaaaattctagaagataacatcagaaaaactcttctagacattggcttaggcaaagacttcataaccaagaactaaaagcaaatgcaacaaaaacaaagaaaaatagatgggacttggccgggtgcattggctcatgcctgtaatccctgcactttgggaggccaaggctggcagatcacttaaggtcaggagttgaagaccagcctggccaatatggtgaaaccccatctctactaaaaatacaaaaattagctgggtgtggtggtacgtgcctgtagtctcagctgccctggaggctgaggcagaggaatcacttgaaactgggaggtggaggttgcagtgatcctagatagtgcaactgcactccagcctgggcaacagagcgagacaccatcatgggaagaaaaaaaaaaacatgggacttaattaaactaaaaagcttctgtacagcaaaagaaataatcagcagagttaacagacaacccccagagtgggagaaaattttgcaaataatggatctgaccaaggagtaatattcagaatctacaaagaactgaaacagcaagaaaaaaaaaatcccatcaaaaagtgggctaaggacatgaacagacaatctcaaaagaagatatacaaatggccaacaagcatggaaaaatgctccacatcactaattatcagggaaatgcaaatcaaaaccacaacgtgatatcaccttactcctgcaagaatggctataataaaaaataatagatgttggcatgggatgtggtgaaaagggaacacttttatattgttgttgggaacgtaaactagtacaaccactatggaaaacagtgtggtgattctttaaagaactaaaagtagatctaccatttgatccagcaatcccactactaggcatgtacccagaggaaaagaagtgattatacaaaaaagatacctgcacatgcatgttcatagcagcacaatttgcaattgcaaaaatatagaaccagcccaaatgctcatcaatcaaccagtggataaagaaaatgtgagacatataaatacacacacatacatacatatacacacacaccatggaatactactacatatacatacacacacacacacacacacacacacacatacataccatggaatactacttatccataaaaaggaatgaaataatggcattcacagcaacctggttggaattggagatcattattctaagtgaagtaactcaggaatggaaaaccaaacattgtatgttttcatgcataagtgggagctaagataatgaggatgcaaaggcataagaacgacacattagactttggggactcaggggaaaagagtggaagggggatgagggataaaagactatatattacgtacagtttaccctgctcaggtgatgggtgcaccaaaatctcagaaatgaaaaaattttaaaaactacaacaatgtcaaattgtttgtattaatcctcctatgcaatcccagtgccacttgaaaggcagtcttcaagtacttctgaaaaatactcatccccaaaacacaggcaaaaaattttgattcagtaacctaaattgtgtgtctggcatttaattgtttgtttgtttgcttgcttaaacttcataagtgattcaatgaatttcgagttttaaaagccaccactttaactgatctggggtttcatgtattgcttcaagcctagaaaatctccaccactatgtcttcaaatataacctctctgctcttccttctcttctctactgctcacactcctgtaagcatatattagagctctaaatctgttcttcccatcttttagttaatctttcacatttctgtccatctttctctctggcctcagggctgtatgatggcttaagtgccatcttctgattcattaattcttcacttgaccttgtccaagcttgagtttatcatatcttttgtgtctctgcttgttattatcaatttctagcatttctactattatatgtatttcaattttgcctgtttgttttataatttatttttatacatatactattatttaaggttcttaaactccttttgaacttctttgttttcttccagagtaaattaatcccccagttactgaattcattaagttgaaagtcaatttattattgcttagcaattgttttcctaatgtgctttgaaattatgatttgccactgtaaatcttatcatcatctctctgaaatgatttattccttttccagtaattttgggggttgccctctacttccttcattcatataagatatttcagggagcaattttaggactctattctgtaatgttttgacattcaatccagacccagtcactaaaccgtcaaaagcataacacatgttctgactacgatactatgactgctttggtttcaagcagcgagcctccaatcccccatctcatatagtacacttctagagaaatcaaacttcaagcccctttctctgtttatagaattggaactcagcagtgactacttatctgttgtgttttgtctactttcttatacactgagatattcatcttgttttttaatatggctttgtctttttaggtagcaaaaaattatatcttcattattgctatgtacttatattctacctagatgactgaattgtaatatcataacaccaacttgagtagtactctttgtgctataattattatatatcatcatataaattctatccagctgaatatataaaaataaaaatttatattattgtttgaaagaggtcaaaagtattgatgtttaaaagtgatatttttatccatctagaaaacacaagagtatcaactccaaagctactttaactaatatgataattaatcctatttacaaataaaatgcacttatgtacacatatattaacagttttcctatatagcagaaataacgcattagaaattaattgaaataatggcattcacagcaacctggttggaattggagatcattattctaagtgaagtaactcaggaatggaaaaccaaacattgtatgttttcatgcataagtgggagctaagataatgaggatgcaaaggcataagaatgacacattagactttggggactcaggggaaaagagtggaagggggatgagggataaaagactacatattacgtacagtttcattcttgttaggtggatagaagtgggaaaagggtcttatagatagtaagtataaagagattggttttggtttgattttgatttacagatattctctctcctttacaacacagttgatcaaaatggattgctactatggtactgagtttctcataaataaaacttttcaagaaaattatggattactatttaccttggatgttgaagtgaagtgaataaattatatctcagattagtccaataagagcagtcattgattattctaaataccctattggacagttccaacaaaataggatgaaaatgccccactaatgcaactggcaaataacttgtttaacaataaatgtgttcttctttctacaagagactgtgctggactttagaggaaaataaagcagcaacaaaagtctgatactaatcccagtttaattctagccctaacctccctaaagaagattacaaatcaatttggaagacaagaacaatgcattacatataaagcagttaaataaaaatttagtaattaaatgggaataaagactatgaagcagaatattttaaaaatgataagtggtaaaacaataagttcagaagtcaagagtgatcctagtaggctaggcgagcaggatatgtggaaaaactaggactactgtttgagctcgaaatgaccaaaaaagtcatacagcgaataactatacctgagcttaaataggcctatagacttttcttgcataattttctctaatttaaagtaacctcaattgtgtgtgcatgcatgtaaatgtgtgtatgttaatgcataaacatatataaatttgtatctataaagaggtataatatatttgcattagaaagaaattttgatagtctataactgaaactataatttctggtagaacttctaagcttcctcccaggaaaatgacaaatcttagacattctttcatgtcttgagttcatccactgtttacttacagtggatgaaataggtgtattctttcaaaattttgcactgcttccaatcccaaataaggcaaaatgaacaatagtagcaacaataacatcaaactgaaaaatttcaaaggaaaattaatattatctaaaattgagcctacgctcaaagtagagttcaagtatagtcctgtatagatcatggagtgaaactgaaacgagactcagaattgacctgcagttgcccttgaaccctgacaaagaacactctaagagaagaaaatgacaagtgaatataattctcaaaatttactaatggaaaaagtttactgatactctttaactttagtggaataaagttttggagataccacagttgcagaataaagacacttaatatccctataaaaacaccatggccatcccaaccactctaagttacttggctgataaaggaaatagaaaaagagagagagggagagagtttttgttttgttttgttttgtttgtttgtttttccaaacttagaaacaaacacattgttctggggctctagaaagatgtgtccagcccgaaggcattcaaatgcaatttttcaagtctaatctggcccagtcatggaccttctttcatttctcctgattgaggtatgtgatacagacagggaatctgcagtttcctaatgtccagatgaccagttttaaatatgaaagaaactaatatttatccaatacctatgctttaccaggaaccgttacgaatattgtaaaaaaaagatttcttcatttgaaggtacatttgacagacgacctctaaatctccttttaacgataaatatattcaattcagaagttttaaatagtttttgaacttgatgcttataaacaaaaaccaagaatgtaccactgagttgatggaggtagggaaagttcagacagtagaatctaaatgaggaatcacaaattcacttaaatgtaaggggattgggtgttaagtgtaaatatgagaccttctcattgggaagactcaatgtctgaaaagggggttgaatttctggctctgcacctgcagcaaatctccaagacttagatcctttgtccggaaaatgggctaattttggcggggaggaagtgaagggtttcactatttatttatgacaaatattcaacactcacaattctctccaatcagaagttctttgagatgctgttggtcttgaccagtcagagaatggagtcatctgactcacccattctgagaatggtcctacagacactataggttttaaattggccattaaacctgcctataaccttgtcaaccttggctaagttcatctggatggatgcatggtccttggcacccatgatgcggttgctggcggaacatttccactgcatgttcaggtccacaaactcaggctggataatatcaattatgttcaattacagagacatggggagagtcaaatgagatcgagtaaaaaatgtgcatgaaaatatccacaatctgtgaaagactatgcaagtgtattattgtcattgaatgttgttagcaatagcatggccattttttacttacaaataaatattgcagttgacatattactaactctaagaaaggttcaaatttacatgaacaaataaaacttcaggaaagaaaactagagttccacttggggactacgtctgacatggttatattaaacattttggaacaattaatttggttttgttgtctctcttctaggatttttaaacatttataaattctcaattctttctattcaatctaaacattttacaatatcctgtggaccttacttccttagtagattcccagaaggtaatgatttgctcctttctgctctccaagcagcttctgtgttggtgcagagttgcagatctctgttggtggtaggttctattctgcagaaaccccctaatcctctgtttaccacaacatgagtctattgccatagaaatgatagctgagatcccagattgaacaccactgacttcacagcagggcctggtaagaggagactctaagctacaaggggggaacgcaatgtatgagaatagtaggcactctgtagccttgaaagaaagggtaactgtcaacattagaataaaaattcatgaaagagctacattgttaggtatgctgtagtttattttctgtataatactgcctggctaaaagtaatcattctgaaatcttatgtcatttaacagtttgctttcaaaaggtctgcagttttccttgattgcatttgtttcttattgaaataagaaacaagcagcttctaattaatcttgttgcacaactgatgtttctgtggtcaaagatttcccttttcatgatgtgttgttttgtttgcgtgtttattgtgtgtgtttgttttgtcttgtttttattaacagaagaaagtgaacccgaacaagatactaaaggtatttttttttctttctgcatggcttctttttaattgtgtagctcttttcaattgtttatttttgttgaccttataaacagctctaagtgccatttttaaagaaataacaaacaatgtatggggaatattaagtcacagcacttaacggtgttattagataggcttctttattatataatatgcttactgaccatttgcatttcttaagcaagattgagttaaaagtaatgtttaaatttggtgtctctggaaatacaaacaaaatgaaatgaaataaatcagcatgagagtataaatcccacatagattggaaaagaaagataccctttgactaaaagacttttggtgggggttggggggctttacagtctttggataaatgttaggatgtcaaggtgccaatttggtttttattgtgatgatttcaacaagatcaggagtattatgcaattgctccaaatcaataaaaattaagttattttaattgcacagagacatatgtttgtagtggaatatgtttagaggaaaatagctggaaattaaatttgtatgggaaaaattctactttttccccaacagttttctgtcaacagtttctgttttctgtaaaaaaaaaaaaaaaaaagaaaaaagaaaaaaaaaaaaggtaccttgcattctgattaagccccaaactttgtgctcttctaacctaaaatttgatatttgggggatgaaatttgtgagttctcatgtaatagagttgactgtaggaattaatctcaaactggtattatttaaaacagtacctccttatcttagtatctcatataaagtacagataatgttttcttatttttaaataccaggttgcttacaattaaatacaggtaaaaaagcattttacctggtcctatgtgaattttttttcttaatttattcctatcagtcattgctggtttgaatcatgtaacctaaaaacccttttcaaaggaagctagaaaataacaaaagaggaagggaaaataaaatccatgggagctccttgtgtcctttgaaataactgctcagaaatagaaattgataagagacttaagtattcctctggcactgctcccttaccaagaaaagagaggtagttcatctccagccaaaccactcatttatttcaggacttaacatggatgccctgtcccttgggctttttattacactccagtgcaatttcagagtgaattatgcctttatctttttactataccttactccttaagatgacaatcaataataaaactgattacatatctgaaagtgctttgaaataaggagagagagagaatataatgtgggtatgttataaaagattttagaattgctggcttcttgagcccaattttgaatataggattctccacttgcagttagcctgcaagtctgctcaagagacattaaagccccaaactcatgaatagatcccatgagagaaaacccagagttaaatatattaccttggtctattttccatgttgcagatgcacttgcttacccacaggaaaccaagtggtttagggctcacagtgaattggagaaggatggataagactatgtacagaaagaggggtttgatgatttttttgtttaaaaatccatatcaaaacagttataaatgctttccaacttactaagtaatttaatgttccttctattaagtggtttaattgtttatgtaacaaaaacacttttatattcttttatgcagataaaacacaaaatagggatattctatgcccattttatttaataagaaacatgtcatataactttttaaattgatgacctagaatgcattttcagattgtgactaccactgaaataatgggatttactgttgacttgtgtccaagtttgttattgccggtgtattattattcatggaggttaatctggcatttatttttaattgcaactgactttattgtttacaatgcttgaaaaacatgtcaaaatttccctcatcacctttttacctttcatatttgaaaaatggtaaatttgagtattatgcaattgctcagatagagttgggtaaaaacatatcagctttattgctgataaggctggaatgaaaaataaagatttctattccatgtgaggattagactaacataacttatgtttcctcatcctcttcctccccaccccactctttccccttgactcttagttgtaggatgaagaagccctgacctggtaaacttacttacacacatcaaccctatatccttcttagatgcaagagagctgggagactgatagctcagaataggattgagatacacataacatatccaagtcccttcccccaaccttaccagagaagtcatcatgttcgttcatttatttattcgttcactcagcaaatattcattgagtgcctaccatatatatataccatgcagggttctagaaatagagacagatatgtagatagatatatagattgtatcagtcagctcatgttaaaaagcaaagcaaaccttgcccttgtgatgcttacattctagagaggagagactgacaaaaaacaaacaagcaaacaaataaatcagtaattaccaaatgattcagatagtggtgatatgatacaatataaacaagggtaaggggaatggggatgagaaagtactatgataaggaaacatttgagtagaaacccagaagacatctgctatctcctttgagaagggaggaagactgggaggaagggtggaaactaactttcaatactgaaaaaagggggttgagtttctattttatcatttagagagcatgtgagagtgcaggagaaagcagcttccctttaatccctggccttattgtcctcttaacctaatccactgcacttaatgaaacccatggtcagccatagcctatctgcccccttcagggagaagtttatcagcagtctcatattcatgaagctacctataatgtcagcagtactcctgtacctgcctgtgccacactggagcccccagaaagttcccatggagggcagagtattacattagttaaattaattccaaagtcacgttgaattgactctgttttatttaccataccacactatgcttagttgtgttgtgcaatgtggataatcagaggtttcacaataagcccaaatgatattccaaatataataacagccaaaagaaaaaaaactgctataaattaggacaaaataaaacctttccaagaagaatttaaagtccccaaattaaaactcttggcttcataaattaaagcagcaaaacataatcgaaattacattattctccaattttttattctgctcatcttcagccagaaggctttcattcaaaaggcgtttttgattcaaaaagttcaagggtatagtcaatgttactagaacagttttctagcacatgtgaggttgaaatgtgctagattttccaaatctctcaaagatcctgcttgtttcagattctcgggcagtgggatccatagatggtcatgcagtagtcccacactcaaaaaatttaggaaacacaagcatagccccaacccctacaagagttattttagccagatgatgcacatggactctaagtctccattattgtaaccagaaaacattagaagagtacctggacgcaccctcactacgatgcctgtcagtgttagttgtcaagagtctgtaacgtgtgaatttagattaagttgggagaataagaatccaagcagaggcacacattgcatttgggtgatatgtctcttaagtctcttttaatctataatggatcactctttcttttttaccccctaatgctaatgattttccctgttaaatatctgggtttgattgaattgcatctccatagtgttctttgaaatgttcctcaaccctttaatttttgtagattgttaattggaccccaggacttacttaaaatcaggctcaattttgcttctgggggacagattcagcttcattttaaacatttgatagcattctttgttattttttaataatctctttttgccttgaagacagcaccttccttgcatttcttttctcctggttttgggacccacatttctatatctaaaatatcaatatcaaaagataggaagtcaaattaatttaaatctgcaaaaagcccccaatagtcaaacaaatcaaatgttccttaatctctttaaattatgcaatgctttttaagaggtactcaacaatgagaccaactggaaacactactctggacctgttctatcattgtcctgcgtatctccttatgtagcacatgtggtctcccatactttagtttagaatccaatattcacaaatttcaatttaactagaattaagtttcaaagcattgtgctaattggggcttcatactctaatattatattctgtgcttaagtcctagaaatcaaaaagtaggagcataaaatatacttacagtataataggccatccaaaagattccgtaatagtttaaacaaacactgaatttgaagtcagaagacttgagtccaggtttctcactcatcattcttgttaccttgggaaaccaacttacttaaattaacaaagatttggttttcactgctataagataagaacaggaatctagccaatctctggagtctcttccaagtttaatgattctgtgaatctaagtaccctccaatttgtaatgcattgtgagcctcaatcccactgtttagttaaaggtaaaatttcttcctacatcaaaataacctcctcttacttaatcagtggggattcagggtcagaaaggaatagaaattactcattctgctgcctacactttatctaaaatatccttttttttttttgcccaatatatcatctgaaaagtttgacacaatttgtctgccctcccactatgatacagttgagggtgtaaagtgtaattatttgattaattcaataaaatactatcctgtgcttgctctggtcaggaactctgcctgaaacaatacagcagtcaacgaaaggcaaggcttttggcttcaaacaatctaatgggaaaggggaataatagacaagtaaaaaataaataaggtaatttcagatagttgtaaggtctgtaaagacaataatgtaggctgatgaaaagtgaatggggggaggattacacttgatgttgtgtgagtaagaaggacctttctgagaaaatgtcacttaaacagagacttgaatgattcaaggaacctactacttgaggatctgggggatgagtggtctaggcagaggaagaacaagggcaagtttccatgttaagaacaagctcagtgtgtccttgagtggtgagaaatatagggttataacataaagggcttggaggctcggccatggcatgtggtattatccttaaaacagagcaaacactgaagaactgcacacaggggaacatggtctaatttgcatttctaaaagaccaccctagcttgaccatttcctgcaggagcgtcactcaccacttctgtcttcctaacagtttttgccaggatcctgcgggctcctgaatcccacaatgtcacctttggctcctttgtgaccctgcactgtacagcaacaggcattcctgtccccaccatcacctggattgaaaacggaaatgctgtgagtgtcatgtgtgtggggacttgtctggggaagacccattggtggtgaacttcaggatagaccatatagttgtagttacccagatctctgtcattggtctcacctacaccacttttcaaagcctcccaatatctttgtcctagaagccattgccatagaaatcaaactccaagagtttgcttccttccatgtatcatgaggaatactagaaaaccaatttctttctttgtgcctttgtgtaaatgggaagtggtaataatatcttcccatataagtaaaataattttcattaaactcatcaccgaaagattagttccctgctagaggctactttgatggaaagtaatccggagttagaatcagtgtcagttcagcattcttttcttcattttcaatagtggtaataataactgatgtatattaagtagttattttcagccaggctctattccaagcactttacctacatacatttatttatttatttattcattctacaaatatgtattgagcttctgaacatcaaacactattctaggccctgtggatacaggagtaaacaaaacaggcaaagttctctgccctcaaagagcttaccttctcatggggaaatacgggaaaatatatgttgtatgtgtatggtgtcaaatgccatgcagaaaaataaatcaggaaaaggagacagaaagttcaaccttaggagaaatcagaactctcctacactgttcatgggaatgggaattactacagtcactatggaaaacagtatggcggttactcaaaaagctaaaaatggaattgccatatgatccaccaatccactactgggtatacagtcaaaagaaagaaaatcagtatatctaagaggtatctgcactcccatgtttattgcggcactattcacaataaccaagatatagaatcaaactaagtatccatcaatggatgaatggatacagaaaatgtggtacatatacacaatgaaagaaatattactaatccataagaagaataaaatcctgttatttgcaacaacatggatggaaccgcaggacattatgttaagtgaaataaaccagacacagcaagaaaagtatttcatgttctctttcataactaaaaaaattgatctcatggagatagagagtagaatgatggatatcagaggctgggaaaggtagtggggaagggaggataaagaggggttcgttcatgggtacataagttagaaggaacaatattgagtgcttagtagctttattagtcagttctcgcatcgcgataaagaaatacctgagactgggtaatttataaagaaaagaggtttaattggctcatggttctgcaggctattcaggaagcataatggcttctgcttctggggaggcctccagaaacttacaatcatggcagaaagcaaagtgggagcaagacatctcacatggccagagcaggaaggagaggaaaggtgccacacacgtttaaacaaccagatctcacaattcactcactcactatcatgagaacagcactgaggggatggtgttaacccattcatgagacttctgtccccatgatccaatcaccccacgccaggcccaactccaacactgggaattacaattcgacttgagatttggtggggacacagatctaaaccatagcaatagcacaacaggacaactatacatagttcacgatattgaactacatagcacaatacaatatttaagtatttcaaaatatcaagaagagtggaattggaatgttcctagcacaaaaaaatgacaaattacttaggtgatggatatcccaattatcctaatttgatcattacacattgtatgcttatatcaaaatttcacatgtaccccataaatatgtacaactattatatatccataaaattttaaattaagaaacattttaaacagaaagttagacctcaggaggaagattatatttttaaaagaatgatcagtgaaggctcatttggacaaagacatttgaacaaagatctgaaggagttgaagggtgaagttacatagataactgggggaacataactagacaaagtctctgagataatggtatacatggcatgtcagaaaaacagcaaggagaccaatgtggttagtggggagtaagcaaagggtcaagagatgagaggcaggcattgtggatcctataggccgtgtggtcatttagacaactttgcttttatttgagtgagatgaggtgccactgtaggtctgtttctgttgtctgttttcccactgattcttgttcattttgtgttgtttccttgtgtgcctagctatctttgattacctgctgtgcattgttttgtataaaaatatcaggaactattttgaggtctagatgaaggtattttcctccaaagagaattttgcttgtttctgccagggacaagaggttactgctaatcttgtaccaccttcaactaagctcaaggtatgaagctccttgggcatcccagataattataacatgaccagtaattcattatgtggactatttaacttgtagttcacactgatagctccctgatatctagttcttttatatcttaatttattatatctcttattagactcttaagggcatgaagttttgatttatctcctgactccctaaggccatccaaaggaaagttcaaattttctgggtttggaaaactctctaagggtaaataagattttcttagtttgttacatctgttaaaaatttttttttcaaaattttaattttctcactgtgtagctttgtctgaataacctaacatcaacatctgagacagaaacctaccatggatttttattgtcattatatagactttaaccactttggtttgcaccgagtctttagtttctgatactctttatgaggttaagaaaatgtatttttattcttaatttgataattatttttaatataaatatattttaaaatcaataaaacacttcatcatcaattacatcctctttttgatgtcatttttcactttaaattgcaaagtttcccttatattgaaccattctttcatttctgaatacattgcttttttgtttaattttttaattgtgttgtttcaattttctgtatttctttcaggattatttgcatctattttcaataacagaaatggttttgtcttttagtaggctatcatgatcaggctttgttaatatttttccaatcttatagaatagactgccaagtttacatagtttcctttgatacagagcttgaggtgagaaagttgagatgcaagtctttcttatactccaagacattttcctctactataccaataattactgcttctctcttctccacatcctttctatgtagactccattgaaatgaatttctttcaaaacttcaatctttctcttctttatgctctgcatatgtttgtggaggaaatagggggccctattttatctgtcaggttattctcaagctctagtgtccaccatgattgtcaactataggaacccacaacagaaaatctcattcgcaatctctatctcacaggtagatgcctcagcctggtgccatatgcatacattatagctcccagagttcttcaattactttgctgactttcttaaagttggctcctgcttacagcccctgctatctgaagtttgaaattttccatgactttgtagggaaaatgtatccattctcctctcagtaaacctcttcttttgtgctaggtttcagctacttagttgatgttaatttttctatctatcaaatcacctctttttatcttccagaatttgctggaaatatctgatcctttgattataataccatctttctatatatttacttttcttaattttgtatttatcttttaggatttcaggtccataaacaagggttaaacatgtgagctaagattattattactgtaactctgatgttaatgaacatgacttgcatagagcactatcttattattttctttgtcatttaaaaatattcagcagagcaagttcttttgctactatctaagcctaccttaagcaggctctgtgatttttctggttactattagatgatttgaaatcattaacattatcccaacttagtcatttaagtgtctagtcctaagtaataaccaaagcatccaatggcattcacagttcctcccttctcagggatatttcagcttaactttcatataagaaaaaattgcagttggcttcttacctcatgctcactcattcttcccccatttcctagctgctctttatgaccccagaaggggtgggtgggaggtagggaggagcaataactggtgcaagagagctctaattcaactgatgcacttgtaagttggctttataccttctgtgcctgccagatatttaaaactgactcttaccctcatagacctattcatgattcttcagaaatcagggaacattaagatgataccagtgatgcattttcctcaggctatccatctttggccacattagcaagtccccaatttgttagttgatggaaagtctctttgtgttggggtctaatcagtcccccaaaaagtcatcttgggaggactgaaaatgactcccaatcagatcttctataaaacctttttattgacaaactctggaagcccagaccattggactctgtcttctttgttccttccattggagctgccagacatcctttagattttacaggtgggaattacatacttgtctaatgtgtctccaaattgcaagtgtgttagagtccccaagatcacattcatgtctgatgattcatgagaaggactcacagattttagataagctgttatgattatggcttattgcaggaaaaggatatagattaaaatcatcaaagaggcatcagttttcctcttctagtgaagttgcatggacagcacttcattctcccagcaacaatgtgtgacagcatgcatgaagtattgtcaaccaaggaggctcaccctaaccttggtgtccagagtttttactggagctcagttacatagatgtggagtgctcacataactagtcttagctagtttccagcccctccaaaggtcaaactaatacaacgtggcctaggattccaagtattcactatcattcacattattatcataaaccatccagcattgcccaaaatctcagatatataaagatattcttattacacaggatattccaaggggcagaggtcatctctcaggagccaaaggccagacctttctttggaatgtgtagggtttgagcacctcgagcccattgagttagcccttcaactttttactgcacagcagtaaaacacatttataactctccgggtggtcccatggaggatacttttatcccttaaggagatgggtgcatgtccaacccagtcctttgggtggattgagaatggctttcaatatggctgaacaacccaaacctttataactcatctaactggtggctattttcagttttcatttcctccaggacaggctgtccaactttttggtatcttggctgtagtgaacatatattatcatattacaatatattaatggtaccatttggagatcaaagaagtgtcccaaatcgggacactttaatatccacatcatcccaaattgtgtttaatgcaagcatcacaacaagaaaatgggggacttctttttaaggaaggcaaaaccttatggtataattcaaatgatattaagtataatttctacccaatccaaggcacattccttcatggacttgaactatttcatgtcttcatgactttctcttctccaggattacattagtatttagggctgccataatagaataccacagatagggtgtcttaaaaaacagaattttttttcctacagttctaaaagctggaagtccaaggtcaaggtgctatcagggttggtttctggtaaggcctctcttcctggcttgcagatggcttcacatggttatctccctgtattctcacagggctttctctccttgtgtgtgcacttctggtgtctgtgcctacaatgatgagcgccaaaaatcccagagtgttttttcataacacagttctcattccaaaatagtttttattcctctcttttatagaccattataaggtttcttcctctaaattaaataacagtgactaaaactaaggcaaatactctggagcaaaataaaaactaaaaaacagatagccttaaagcaggaagttaccctgtcactagggcttggcatactgtaggctttagaagcatttcgcggcatgaaagaatggtaaccctgaggcatctccatgtaataagttaatttgtatactgcttttaaataaagttaaacaacttacagtttcagaaagatagaagggaagagcattcggaagactgggagtgaagtttcccgggcctgaacccttattcaaaggcgtctcgacatggataacctcgagaacattatgctaagtgaaataagccagatacagaaaggcaaataccaaatgatctcacttatatgtggaatctaagagttgaactcatagaagcacagaatagaatagtggctgtcagggatgggaggatggggagatgatcatcaaagggtacaaagtttcagtcatgcaagatgaattaagtactgtagatgtaatgtacaatatggtgactacagctaacaacactgtattgcgcacttgaaatttgctaagagggtagatcctaagtgttctcaaaacacacacacaaaggtaaccatgtgagatgatacttcactagcttgattgcagtaatcatttcataatatacatatatatctcaaaacatcaccttgttcaccttaaatatatacaatttctatttgtgagttataccaccaataagctaaaaaaaattaaaagtctaaatttttttaaaggagtttcaaaaactaatgctttgctctatacttgtacctggccccacccttgtctctctaaagcagggtgccctaggaagactgattggctgtagccccaaaaggtgagaactaggacataagataagaaagggaagaagggaaagggagagaaggaagtggggaacaaagtgcgtcaatgtgtgaaattttagttggatgttgtaaaacaggaaaaaaaatgccaaattctatggctaccccaataacaaagaatgtcttatggcaatggcaccctccaagtcatataattcatttattattgtcactttcactattgttttcgctgaagtcttttttctgttacttgaacaaaatcatttttttcgtaatttgttaataaaattgtttcattttatctgtctatcctacatcccttcaaatacagtacacaaacaaatgaacgtgcttttccagcttgtaacttgaggcatggtagaatgagaattagatggggagttggaagactgaggttgtaaatgcccctctttagttaatgtgattttggggtccgtttacaactcagacactgattttctcatatctatctacactaacacagacgtcaggtggctgtcaaagatgatctctaatggtataattagagacatcagggctaatgttctctaattttgtccagttatgataaagtaacttggagtgcctgttctttacctctctaagaacgtttcaagtgtcatttaaaatttgattcttatttgtcaatgtgttttttggctcctctgtggaaactgttatagtcttaagcctgtcaagggagtataaagacatcctaagaaaatttggagactgggccgggtgcggtgactcactcctgtaatcccagcacttttggaggccaaggtgggaggattatgaggtcaggatggagaccatcctggctaacacggtgaaaacctcgtatctactaaaaatacaaaaaatttagtcaggcgtggtggcacatgtctgtagtcccagctacttgggaggctgaggcagaagaatcacttgaatccaggaggtggaggttacagtgagccaagattgcgccactgcactccagtctgggcaacagagagaaactccatctcaaaaaataaacaaaataaaattgggagactgacaatataacttcactaatttgtctatgtaaaaatagtaataaagaaaatggaaaggtatatactttttaaatttctagtattttcaaaagattcatatgaggttataaatggaagataaatgattattctctgtcgaagtttaggaataacaagagtaggatagtttaagatagttgaggccttattgagaccccaacttattcattcaacatgtgactcacaaaaagtgaaaggcaaaatagatttatcacctcagattctaaaacaggaataaaacaaacaaagtgactttcatccaggaagaaagcaaaagtacatccgcagttcttacggtcagagagtgtgtgctgctcttagagccaagtttagttcttggtaattctacccaagtctgaaaggaaatcaagatgactgtccacattgacgcaaatcacaggacatggaggcaatgtcaggcatttaatgttgtaaaagttgcaaaccagaaatcccatggaggagaacacagagaatcctatagtcctgatatcatttttggtggaatgaacaaagacgaacgcagagctgggagatatttggcacagaatgaaacagagtttgaactgtggctcttcactcactacgagttagtaaactaatgccatagcacgctgctcagtacaatgggtactctgaaagaagtgagtgctccctttctcccctgctgtgaacctgccagtgaaccaaaacccttaacagttagagccaaagagcaggagaaagtccagcaacgtgtcccaaagtgattagaaactatcttgctttcctcctatggaaataccaagcattttttgtggaaatctgaattcacagatgaagttcagagtgaatagtaacattccttttgttttacaatcttggaaatagtagtttggcatggaaagagcagagcttactttttaaaaagaaaattatttgtatttttataaccagatttatttatagttataccagtaatatctctgttattttggcatgcaactaaaaaatatagaaaaatttcatgtccctcttgctttgtccaataaatataatttctaaaatattgtagaagttgaataattttaacttgttagaagaattttataaaataaacagccaaatcacctaggttgaaggttgtcttttagaattttcttaaagggaggcatatctatatttagacatatttttaagaggcagtagttttgctttaaaatatatgacctgtgaagtccttttatcttactagctgtgtaaaccaaaataattacatgtaacaagtcttatgtggaataattcagcagccagtttgaaagaggctaattaatatttgtggctacattaatcagaaaaactatggaaatgtctcttggtttattatccctttttctatgattcataaacagacattattacaactgcataaacatgaaccctactgggcaacatccttttttttttttttatttttttccccaagatggagtcttgctctgtcatgaggctggagtgcagtggcgcgatctcagctcactgcaacctccacctcctgggttcaagcaatgctcctgcctcagcctctggagtagctgggactacaggtgggtgccatcaagcccagctaactttttgtattcttagtagagacagggtttcaccatgtgagccaggatggtcttgatctcctaacctcgtgatctgcctgccttggtcttccaaagcgctgggattacaggcatgagccactgcacccggccctgggccatattctaacagttccacaggatatttccagccagctggaactatctgcaagacagggccattgaggccagaatacctttcagaggtcttccctagctaaggtcaaatcataaaattatcggattatccatagctgggggtggattctctcaagaccgcattagtaatgtattaatgatacattaagaagtactccacaggtctataactgctcaatcaccttcaaagagtttttacacacattatgtcaggcatgaattattatgcctatttaatagagcttcctatcctactgcctctttttagaaaaaaaagttttattgtgatctttattgtatacatttaaggtacaaaacatgatgtttagataaacatatacagtgaaatgattactatagtcaagcaaattaacaagtccatctcctcacatagttacagctaacttagaaagacagaacatcacacccagagcagggagctgtgtctccagctccctcaggtccatatacctgagtgacttggagaaaatcatgcatttcctgtaactaccagacgtagtcacctcaattaagtaatctgttttgcttaggcataccttggtaaaagctcacagtactaatctggacccacccctttgatctttgaaacctgaggtgcgaaaaaatatcaaaatggagggcagagggatttgtttttattttcagatcatcaaaaatactaaactgttgatcagaaccatccatgaagaaagaaaaacaaattcaaactatgactaatggggcctgctgtgcgtgagtcacaaactgaatcaagcaaggcctctggaagcagggggaaaagcattcaggagtcactttgtactcaagcatgaacctcctaaagtgttggtctctttgctgaactatttcaacacctggatgaagtccaggagaagagacgtcaggcagtttaaaaattgcttctctttcctactaaggatgttttcagtgaccttgaggaggtgtttaagaatgactttcttaggtgtgaacatgaaaacagtgggacttagggcaacatttggggagattccttgcttcaaagttgtctctaaggcattgcacattcctacttcttgcaggagggaactgtctttccaaaggattttgcaaactgttctgcttttgcttatctaaactggaaagagatatataaacaaaatataaaatacaaaataacataaagtctgttgattgagcatagaggcttggggtctttagctttttttcttattgttgctttgtattaaaattcaagggagtccaaatattttcagtccccttgatctaactgggatactcatacaatgagacattgcacttctgataaatatgataatgatgagtttagtttgaattcccaactgagtgcatgtctcttaaaaacatttttctcttcactttgtaaatttcttatagagtctaggttgctcatctggggaaaatgccacaatacttgatctgacctggaaagattcccagtgaaggtaaattgatactcacataagccagtttttatcataagaattgagactagtactgaaggtaaagaataaaaagctagttggacaaacgttcactgcatgattgctacacagtgcttggtgccagggatagccagccacctggcagctaggaagaaagctggccttacctcagagctttcttcttcctggctagtcaaactgtgaaaatcctttctcatgcaacacaataaaataattggctccaaaatgtctcattctcagcctcattccctaatactggacacaggaatctctttcagccaagcagccaagctcaggccaacatgggacgtacctccactcctttaagataaagtcaaagcataacaccagctgattccttaactgtcatttcactccagtttccctcaattggcatcatgtttccctgatataagcagcgttaattaaagattagttattgaaaaggtggtattgtgctcaagaaaacagtgaaaaaagaaccaccatttaaaaaaaatgccaccaccttaacaatgacatttgtcagatatgtattttgttcatggttggttgatttgttaaggtagttgttactcttgttttctaatgactgctcatttgttctctcttctaggactagggaaggcactgtaacataatgattaaaagcataggctttggtgtcagacacgctaggatgcaaattctaactatgcaaccttgtaaaatgtgctgggcagctatctttcctacagagttggagtaatgttgactaatatatatgcatttgtataaattaagctgttaattgtgtgtcaatattgcattgatatcttggctctttaaggacatttgattttctggtttaagtagtacctcctgcatgaacttcctgacgtgttgaactatttcaacacctggatgaagtccaggagaagagaggtcaggcagtttaaaaactgcttctgtttcctactaaggatgttttcggtgaccttgaggtattatgtatgtattatgtatgtcatgcctcagagacagacttggcttcacaaccatttttcctgacaaccatccacctatctgacatgcatttatggggaactgaacaggagttatgaaaataagtccttaagtctgcaacctctcatacttaactgtcatctgtaggacactgaagacatatatcagaggatccacaccgtatataattcatgccttgtattggttatctatggttgtatcacaaatcagcccaaagcttagtgacttaaaccaataactattagtgctcacaagtctgtggttcaactgggtggttttgctgatttggttttgctgctgggcaggactgcctgatcttggctggtcaagttcctgtgatcggcagattgaaaaggtggtagttggttcagaatagccttatgcgtaccttggatggttgttgttggttcccacctggggcagaggggataaacgggccaggtgtgtttcatcttctaccatgctggctcttgcttgtttataggcagtcccaggttccataaacaagaaagcaagccacaatgtgcaagaacgttagtttctgctcattcattccaaaacacatcacaaggctaatctagattcaaattatggaggaaaatgctacttcttgagggaagagctacaaagtcacattgcaagggcatggacacagggagggaaaatcttttggctatgtttgcagtgtaccacatgcacttgattataatcttaatgcttttcccttcaaatctactgacatagtatagtgggaaatccttgactgagttcttttattttcctttactctgtcaggtttcttctgggtccattcaagagagtgtgaaagaccgagtgattgactcaagactgcagctgtttatcaccaagccaggactctacacatgcatagctaccaataagcatggggagaagttcagtactgccaaggctgcagccaccatcagcatagcaggtaggatgccccttcacatttgcgttgttccaggagaggggagagttgatactattctacaatatcgagcattggagaaaatctcccttttcttttatctcttatttcagttgcatttgggctatttcctcccccatggggatactccggaggtgacctagatatggattttggcagatttgctgcctggaaaactaaatcctcaatacctacccaagtggtaggtaacagcttccaggaactcacttcttccttccttcttgtttttcttatcttttttccttttctccatttctctttcctccctccctccttccctccctcgctcccttttttccttccttctttccttccttcccttattccctccttcattcctccctccttccctccctccctcttcccttccctccctccctcttttccttttctttaatttcctttccttttccccactcctcgttctctcttttggttgatcaaactttccaagtctctcatggagaaagttggcagacattcaacatggtcatgccaatttctgcatggctatttcttgcgggcatttttacctgtgagcatgatccctgtggtctttcccttccctgcttcagcaagccacagcaggtcttacaggtgttttcaagaaaaaggctgacacacggatttctaggatgtttgtgttgagttctaaaggtttatttttaaaatgtaaatgtcccaaaggcttctgagaacatttatagaattcctagagatggaaataatccctgtatcaggaaatgccaggcatattagattcatagcgtgcctagggctcaagggtcaggagagaggtgcttggcctgacatgtaaggcaagcacggatagagcttgtgcctagggccaactccccacagcctgctgtttcttattacagtgccacctctttgatcttccctgatggggaactttccagtaatgtctgtgatttgtaatatgaggtgaataaaaaccacagataaatgataaggagcactggacaaggctggccctctgggactgcctatgaaggaaaaggaagacttcaaaattaaggttccctctcctttctccccccagtgatatctctgcctaccatgtcttcagacaccatatcagttattctagcatttccacaggtgggaaattatttctcccagaaaatgaaaagtcttctaaaagtaaattcaactcaatttaacaaataataataatatcacataataagtaatagtataatcatataatataaatagtaataagaattactatttacatagcactttgctgtttataaagcacttttacatgcatttatcaaatactctagatgacgcccattgtgttgggtagagtaagagatgaaaagttaatttaaaaatgggtcctgccttccagggactctgcatctagaaggaagtcagtcagtcacgtatattatggcactggctgtattctattgcagaagcatacacaaaatacaacagcagaacaagagaggcaggatgtcagggaatccttggaaaagaggtatcattggagcagaagatgtggatatgagatgactaggttgaagtcaaacctttcaaacctgttcaaaaaacagcaaatcagtggtcaaggctagaccacgagagagacagcagggaggtggaaagtggaaacaaaagttgaaacatgggctctagccaggttatgaagagcctgcataggtttagtatattatttcattgatgagaagtcatttaatgatttagaagaaataagtgcaatagatatatcctctagcaaattaacagatgctgaggtgttaggaggttgagaagtgcggcaagctgtcaggaggctcttccaggtgtctcaaaaagtcatgaaggtctgaacagcagcagcagcattggttcaaagggaagaggagagatgcacaggacaaatcaggatagaaataaggtttgcaaatgcttgaaaatggcagtgggctagaaggatgagttcagatgtgtctgattttgaaaggtgcccaggacaagcaataatatataaacacaaagcatatgaacaggagaaggtccaggagataatagaatactcaaaaatgttgatgcctttgaattacctggaattattctggtctcactgtctttgtattatttgacatttgatcaagacctactgttaaataaacatatatttataaaaatctatcaatatatatttatataacatatatgtatgtatgtgtgtttatatacacacacatggagagagagagagaatcacatttaaatatgaatactccatccaagaaaataaactgatctactttaatttaacctgcactttcactggcagaagaaaaatactaatgttactaatcttaggagagtctcatttcaggtcattattcagtcactcaaagatacccgtgccagagctctttctcccttttcttccttctttctctttccctagatggcaaaggagaagaggatttgtatttaggcagtggtgtcttctggacaccccccacaccactgctaacccccggatctctagtgaagtctgcagctgaacttgtgattttatctcttggcttggttagggcccaggggacctccctggctggcttagcctgtttgagacatcttttggagcagcacagccctctgcatctcagccaaatccttcatcatgccattgtctacactctgctctgcaacctcagaagctaggtctccctgtctgtccctactgtggccctgtggctggcctgctgaattccaactcagctccttcagtactcctcacagggcctgaagcaacttctagatcactttcaaggcatacagaattcaggagagatttgggcttgctaactcagttctgctgtctgcccaaccttgtcatgctaccattttcacattaatgttgccatccatgttggagtaagggatccctgtggggtgttttacttatagcatcctaaataagaagttagaaaaactccatctgcttttggctttttccttaattgatcaatacacctctattctaggatttgcattcagaaaagggatctataaaatctataaaatatccaaatttttaatctccctaccttcctccttttcacagcatatgaagctggaagagcaggtggtcaggaatctcccccaaccttcagtgcagtaatctttagagcctactcatcataagtacaaaggtggttgtgaaggcagtgctataaccactgaggggaacctaataaaatgggtatatctactccacacaagggaagattcatcccatggacacaatctttaagatgccgagaggactacagggcatgattgccaccatactatatgcagcaacatcatgacttcattcctgtcctagcccagatgagaacctgatccaactccataaacggaacaccaacagggaaatctgtaaccttaaaaaaacggcatcttccagtggtagaaagagcctaaaggtctgagacaggcagtataggaaggccatgtcctagggtgtagggagaggtcatcttagatatcagtgttgagaaagaccagaatgaggatggggacaatcagcaatggccacccaggaaaaataacagtggaatttgcatgggggaagggatggtgggagcaatgcaaagattggcatgtggcagcagagagcaacttgcaatctgctgcagcagcagaggtgagtgcctatgaagagcagttatctctgtaggaataatggttgtgtagactagaaccagctatgacttttcatgagcttgacaatggcagtgcgatggaggtcagctttagagtggggagaagttggggaccctggaagtgaggcttgaagccactctggacaggggtatgtttacagtagctacccaaggaacttgggtagaggtagagaaggcagcataaggagagggttaacctataactgggacctgggactttgctcttctgaaccacaaaggcaacaattaaatctgaccatatacagaatcatagtgctgcacgatgttcggagtcaagtaaagaaatgagagttgttactcgaaaactgaaaaatggaaaagactaaaaaggtctagagtggatatgataaatgagaaacatgcctattaattaaaaaattgctacacatctagtgttattccctggaataagacccttttgtccaacccctgatttgtttgtctggttgattctcactgattattaggggacttgacactcctcgacttttcccaaaatttcccgaggcagacctaataagggtgtgtcatgagtgtgattgaataagacttctgaactcccatgagacaacttccattttctcttcctctttgacctgctcttgtgagttagacctgctagaagccccatgaaggtccctctggaatcactgaggaaactacctcccaacactggggtgctccgtggactgatattttagggaaatgaggctacttcgatgaaaaagggatccttggagcctctttcattcagggtcatttgaaagaagcatggccttatgctccccagatctcccagaactcaagtatggtatctgctttctccatttctgcttttggcccctctctcggcatccgatgaggctggccagaagtccagaacttcaagtcagacatggcgcctcttttgccattctggcaatgcttccttaatgccaaactctgctgtctcatttaaccatctccatcctcccctatgtgactagtacccctagtagtcaacagccccctctgtcatgttcctcgtgtttccctttgaaatttctactccatctgtttatttgctcttcccagcaggaaaactggaaatggaaaaagctgatcttatgcttatgcttaggattatgtctaatgatatttctagtgtttgaaggacaacaaaaaataaagatcaggcttcatttttccaaaacccttccatgtgtctgcccaaatccttatgcattgttttagaaatgtgatatatttctttctgactcatttacatttactgttagtcatcaaaaggagtcagaaaaacttttggaacctctccctatgaaccaggaagttgcatttcccctccagtcacaaccccataaagcctcaagttgcaaagtaaacttgccaggatgctgggcatttggcaagatgcttctctttgtcatcagaaccctcagccctgtggaagaaagcaagacagcctatgataccaatagtgagcatagtcctaaaacagccggagaccatgaaaatggacacagggattcagaagagaaaaatacaggccgggtgtggtgactcacacctgtaaccccaacactttgggagtccgaggtgggcagatcacctgaagtcaggagttcgagaccagcttgggcaacatggtgaaactccatctctactaaaaaatacaaaaattagccaggtatggtgacgcatgcctgtaatcccagctacttgggaggccgaggcatgagagtcacttgcacctgggagatggaggctgcaatgagctgaaattgcaccactgcaccacggcactccagcctgagcaatagagtgagacttcatctcaaaaaggaaaaaaaaaaaaaagagagagagagagagagagaaataaaatacatatttgtatttcccattctcaatggcaccaacaggtccttcatcagagagtgcagtccatggttctatccatgtttatccagtctcagagtacagggacgagcctatggtcacacacccctctaatgtaagccgctccccattctaccaacaacccccactttctatattgccccttctttgaatcgacctcagggtctaagccttttggtcactaaatttaaaagtcacatgaatacaagtagtatcaaaactgagcaggagggaaaaatctgatgtggtaatactggagtctcccaaactccccagatgaaccacatggttttatttttcttatttcaaaagaggtgatgtttcacactactacagaaaaaaaaaatggaactttgaacactatttcataatcagtatagctgattgaatgtactgacatgtttcatgctctttctcatgtctgatagccacactcctgtttcctcccaatgttggagataattcttgagacttggtaacagagcagtagcacgtaatttgctttaccactcagtgcttcaaccatcataggtaaagagtgttcttatcaacaaaacagacaccatccctggcattccatgctggctttctctggttttaaattaattaaagtcattttgtttttgttcttaataaaagggaaaaggataaatgagtaacagccagtagccacattaaattgctaccagatgttctgcagttaggcataaaaggtaagcatccttttacaatcaggcagctttcttatattagaacagttctgtttcagaagagagccaatgtttttgctgacttctagataattttaaagatactataatattggtggatattttaagaatttttaaatactacaaatattggtgggatattttgagaattttttaaaattgaattctgagcttaaagcactttatagttgcaaggttcttggaccccttcctcatctcctagccatacacccccacaaaaataaaagaaaatgcaagtggagacatttcctatagaagctcattatcccctcccaggtactatggctggattctcttttgcattggcttatctccatgctccttccacattacaaaatgagggctctatcatttacataaccttggcccccttgcttttcaatagatcttcctgctcagctgctattagaatctgcctttgtatttccaaattatatgccattttctctggaagaattttccagtgttccactgtctttcttcccactattgtatttgtataatggcagaactgccatgagatttgggggcatgaaggcacttctttcctgagttcagcagtgacatagagttctcttcattctctttcatagcctttggtcattttaattaagagactcttgcaggcctctagcccggttcactaaaatttaaattctctctaatttataatttgttgacatcattggcatcacctcttagatgatcaaaatgacaatcctataacccagcctgcctatctcagtaaaacagaaagaatctactagatgtttttcagaatgtaaatagatataacatttataatgattttcctattgtccagcaaattatctcgcagttggagtcctgcaaatgttagagacatcttgatgtttccttcatagtcactccagagtctgttagttcttatgtataaagaaatttcacaaagaaatccagcttttctcactgctaaaaggatgtggccttctgtccctaagcaaagtgacccatttaggggagggataaaattttagagcttattattggctaaaaccctactctggatcaaaagtttgtttttaaagtgcattgtagattgcagaacaactatgaatcactcataaagagaggtaacaaagacctgaggctatatgtgtaaccagtgtattgaacaatttaaacctgacaggtacacagagaaaatggtgttttctccaaccaatggggactgaggaattatgccttttagaatctccttgcccttctcaagagcaaaccctttacaaactttcaggcagattccataatttccaggtaatgtttagagattaaaaacccagcctgatctagtttaaaactctcctcttacataatttaaacctcatctgcttcactctctccactgccagccaatcttccattcaggaacatggagagtagacaaaatgggtctgctactattatacacccttcccttcttccctttcctcctgttcctcttcatctctggcatatttaaggagaagaggatttacaaggaaggcggcaaatgtgttttcctaaatggaatatgttttagttctttctctgttaattgttattgcttctctgactaataataataatgttaacaattgcaaggtacattctcagtgcccagtgccatatatatatacatatatatatatatatacacatatatatatacatatatatatatacatatatatatatatatatgaatttatttaacacccacaactcaatgaaatggatacttttattatccccatttcacagatgagcaaactaatgctcagaaaggtgcccagtctgacacagctagtaaatggcagaaccggtattgaaacccagcaggtgcagcatccaggcttttaatgccttgatgggttattaatgtctctctattgcaggtggtacctgaatactgactgtctcctaggacatttgggttctttggagggtccttcggaagactgtgtattgcacagtgccctgatgtgggagattctttactggttcaacctctcataatccccagtccttgatctcaatccctattgaggtaacctgcaattgcttatgactggggttctttgtctggtgtccagtcttcttggtaacttagctaccttcatagcatccactgaacacacaggaatgtcccatgtccctaccatcccaatggtggggagttctattcctggaccctctcactctgccctcagattgttgcaattctttttagacagtaatattattaaaaaaaaaaaaagacaaaacaaatgctttccttgtctctgagtcctagtctcttaattcttaggcagttgaaaaatgactccatgagatgtgttgttaccatttacattgtatcattcaagaatgagaagatctagggaaataattcatcccctttctccttatatttgctgagatgcccttcatctttacttgaaggctgagaaccaaagctatttgtctggttatagggcctacaaattgcaacaaaggttggggtgattgtgccttttagtaaattccaaggaaaaatgtcaagttgtatgtgtgtgtgtgtctgtgtatgtttacatatttcctacgtgtgtgtgtctgtgtgtgtgtgtttacatgtttcctataatcatttgtcttgctttttgagggcattccaactactggtggaaaaaataaataaatacaagggggtccatgctgtgagtagttgaaatgaaaatactggtgttttgatgggattaattcagtacatttataatagactaattcacccaaaatcaacttctaaaaccagtttgccttaactcacttaactcaatttcagttagcctgatgactattgcatctaatgttgaaaattcagaattaaaaatgaaatattggctggatacagtggctcatgcctgtatcccagcactttgggaggctgaggcgggtggataatttgaggttgggagttcgagactagcctggccaacatggtgataccccatctctactaaaaatacaaaaattagccaggcagggcagcaggtgcctgtagtcccagctactcaggaggctgaagcaggataatcgcttgaacctgggaggcaaaggttacagtgagccaagattgtgccactgagctccagcctgggcaacagagtgagactctgtcttgaaaaaaaaaaaaaagaaaaagaaagaaagaaacatttaccaaataatatggctgagtatttatgactaatttttgtattgctaatactttatcacatatagggaatcatgagattgttacatattgttgtgactgtctttttctgttccagtcattttgaacacttgcattattctttagccaatttgtcaaaaaaaaaaagtctttttctttacaattttgaatttctgctaaaaatatttagtggaatgaaatttattccattaaaaaaacgaaccttttgattattattgtctccttgctactttcctttattataagagtctatgaactctacatgtatgtgtatatatatacatatgtatgcattaaccaaattagcatactctcagagctcatttctgtttagtgaggtattcgtattttaaaagaataaagaaaaatgggtccctcttttgatcaccattccatgttatttatcaagagatgtaattgataaaggttttattttgttttgttttgagacagagtctctctctcttcctcccaagttggaatgcagtggtgtgtgatctcggctcattgcagcctctgcctgagctcaagagatcctcctgccacagccttccaaatagctgggactacaggtgtgtgccataacgcccagctaatttttgtattttttgtaaagaaggagtttcaccatgttgctcagactggtcttgaattcctgggctcaaatgatcctcctgcctgggtctcccaaagtgctggaattacaggcatgagccaccatgcctggccctaattgataaagttttaaaatgcaaaatgtgcatcatttccaaatgaatttatagtgagaatcataacattttgaaaaacagttttcaaaatgccactggagtgttaacaaattaataccatctgtgatgacttttccattctgccaaaacctgataaagctctgctgaagtaaagaaattggtttcacagagccagcacactgaatgggcaaaagctggaagtattccccttgaaaaccagaataagacaaggatgcccactctcaccactcctattcaacatagtactagaagccctggccatagcaatcagataagggaaagaagtaaaaggcatccaaataggaagaggggaactcaaagtatccctgtttgcagataacatgattctatatctagaaaaccccatagtctctgcccaaaagctccttgagctgttaagcaacatcagcaaagtttcagggtacaaaattaatgtacaaaagtcagtaaattcctatataccaacaatcaccaagccaaatcagaaacacaatcccattcacgattgccacaaaagaataaataccgaggaatacagctaaccaggaaggtgaaagatctctgcaataagaattacaaaacactgctcaatgtgcttaaagcaatttatagattcaatgctactgctatcaaacaaccaacgacattcttcacagaactagaaacaaaactattctaaaattcatatagaatcaaaaaagggcccaaacagccaaggcaaccctaaacaaaaagaacaaagctggaggaatcatgctatcttacttcaaactatactacaggactgcagtaaccaaaacagcatggtactggtacaaaaacagacatatagaccactggaagagaatagagagctcagaaataaaaccacacacttacaaccatctagtcttaggcaaagctggcaaaaacaggcaatggggaaagaatccctatttaataaatggtgctgtgatagcaggctagccatatgtaaaagattgaagctggatcccttccttacaccatatacaaaaatcaactcaagatggattatagacctaataaaacctaaaacagtaaacctgaaagataacttaggaaatacattttggccataggaactggcaaaaatttcatgatgaaaatgccaaaagcacttgcagccaaagcaaatctgacaaatggaacttaattaaacttaagagcttctacatagcaaaagaaactatcaacagagtaaacaggcaacttacagagtggagaaaatatttacaaaccatacatctgacaaaggtctaatatttcagaatctacagggaaattatacatacaaggaaaaaaacaaacaacctcatttaaaagtgagcaaaggacataaacagacacttttgaaaagaagacatacatgcagccaacaagcatatgaaaaaatgctcaatatcactaatcattagagaaatgcaaatcaaaaccacaatgagataccatgtcactccagttagagtggctattattaaaaagtcaaaacataacagatgctgtctgagaccagcctggacaacatggcaaaacctcgtctctactagaaatccaaaagttagccaggtgtggtggcacatgcctgtaatcccagctgcttgggaggctgaggcaggaaaataacttgaacccaggaggcagaggctgcagtgagccaagatcacaccactgcactccctcctgggccacacagtgagactccacctcaaaagaaataaaaaattaaaaaataacagacactggcgaggttgctgagaaaagggaactcttatacactgctgctggtggtgtaaattagttcaaccactgtggaaagcagtgtggtaagtcctaagataacaaaaacagaactaccattcgacccagcaattccgctactgagtatatattcaaaggaatataaatcagtctaccataaagacacatgcatgcaaatgttcgttgcagcactattcataatagccaagatgtggaatcaacctaaatgcctatcaatgacagattggacaaaggaaatgtggcacatatacaccatggaatactacgtggtcataaaaaataataagatcatgtcctttgcaggaacatgtatggaactggaggccactatccttagcaaactaatgcaggaacaggaaactaaatactgcatgttcccacttataagtgggagctaaataatgagaacacatggacagaaagaggggaacaacagacactgggatctacctgagggtagagggttggagaaaggagaggattaggaaaaaaaaaaatagaacaactttcgggcactatggttagtacgcgggtgatgaaataatctatacaccaaactcgcaagtcacaagtttacctacacaacaaacctgaattatgtacccccgaacctaaaatcaaagttaaaatattcttcaggatcactgaataaaaagctcataaagattcaaaaaagtcatacactcaaagttataaaaaccttctagttaccaaccctcaccaatctcaaagagaatgattagaaatacaagctcattgtattttaagaagattgacctctcagagaaattcatgttacagcccttaaaaatcccccccaaaataacacccaaatggatctaaaaccttcaaaacggagttagacctcaacattaggagaaattttaatatggcaaattggttttagacaaagcatatgttttactgtttattttcgtaatgagggttgagaggacaggacgaagtaaccataaagcacttggtaaagagtgtgaagtttttaatttacaagaaagaaaatggaataattcagaaatgctggttatatcacacccttgccgggattctcatgccttggcagagtctgagttggggctgtttgtcctgggaatcacataccaccagaggagaggcgtattctcctcagagccctgagtttttacatccgtgagcatgggcagctacataatttgcagggctaggtgttccaaaaacggtaaggatttcaaggtagggagagcagaacattaaattaagcccgaggcccttctgagtgcagatgccatgcagctacccagggcacacagccttgaggcagccaggtctatgagaacaaccattgcctactttctcttctaccctctctcctctgcatgttcttttcttttctccttctccttcttttttctttttttccaagtctgctttatgcatcttctctgttaacctccacatcttgcttttttttttttttttttttttttttgagacggagtctcgctgtcgcccaggctggagtgcactggcgcgatcttggttcactgcaggctccgccccccgggggttcacgccattctcctgcctcagcctcccacgtagcgggattacaggcgcccgccacctcgcccggctaattttttgtatttttagtagagatggggtttcaccgtgttagccaggatggtctcgatctcctgacctcatgatccgcccacctcggccttccaaagtgctgggattacaggcgtgagccaccgcgcccggcccacatcttgatttcttaatgtaaagtccctgggtagctaatggagggaaattctgagcttagatattgccttggtggatgttacccatgattgactcagtttgaaaagaacctgcttcctaaagatactcaatattttcttcatttcctccgaatctgcccattaatgcagcatctggcctcctagcagaagcggagaacttttcagaaacttagagatcagtagtatcaaaacaatctcagccaaagcataagatgaaaatgtactaacgttctttcttttctcctttaatttgacttcctgtgggaacttcctttcctgttaatagaatggaggtaagaaactgttattgtaacaattgtttccaatgttttgttttgtagggatttcgtttgtttgtttttgtctgtgagagggtcttgtgttgcccaggctggagtgcggtggagtatgttttgtttttgttatttgacatgccatttattgagtgtttactatttgctgagcactgtaacattttagaaactttctaatttctaatccttttagtaagtatgatgaagtgaaagacaggtaacctctactgaatacttactatggatcgggcactgtgctgagcttcacatgcattatttaaatctatcttcaagacatccccatggaatgggtattattgttatctctattttgcagaaaaaaaaaataaaaaaacctaagaaagtttctttgaactggagagttcaagtggcttacacagccagtaaatgataaagttagaaatggaacccagaactgccaaactcccccttatagctctaacctatatataagaaggtatcccttgcaatataaccttttgcaacttacagtgttttatttgcagtacttttaaaactgaccttcataaaactctatggagtgtaattaattttaagacagcattcttagcacatagcctacgtttaaaaacaggcagcaggatacaaaattttggttagatagcaaaaataagttcaagagatttagagtacaatacagtgactatcattaataacaatgtattgtattcttgacaaccactgagaatagattttaagtgttctcatcatcagaaaatggtaagtatgtgaggtaatgcatttgttaattagctcaatttagtcatcccacaatgcgtacatgttttaaaacatcatgtggtacacaataaaaaaaacagttttttttaactaaaaacaccttttaaagttttactgttaaaaaggtcttaagaaataccatttggatgtatatattgctctgaaaagcagtgaaaatcatgatacacattatatgaaaaattaaactagttttaagtatataggatagtgatttgtcataattgactgcattccagtttgttaatgtggtatcaaaaattctcagttcaaaaaaaagaaaaaattctgagtccattttttaatctaaatattgctttaataatttttattaacaattttggagctactgccttagcagagtaaagtttatctaaactaaaattaataaaagtgccatttaataaccctccttctgaaaaaaacaggcagcaggccaaatttgtcctgtgggttgtacttgctgacccctgccttggagaataaattatatacaaagacactagacgcgaaagaatagaaacagttcccgtttgctttgaaggttggaagtagcacagaatctgtcattacctttgttgggaatgttcaaggagagataaggcagttgctaaaaaagggaagatttgtctgaagcagaggagaggtacagctgtcaagtgtgcctgtgcagggagggagggaagtaaggtgtggagtttccactagagtcttggggttgtcataacacaaacatgccatggctggtcatctggaatgcccttgggacaaaatattatgtgttcaaagggagtagacatctccctcagagaaacttgaagataacattttaaaatgttctattgaatgtgattcatgcgtttcaaagtagagagaaataggagtaagaggtgcagtgagaattctttacctactccctttgaccacatagggttgtctgttataaaagaagttgaaaaggagatccgagatcaaatatccattattgcctctacaagaaagggaacttttctgaaagagaagtcaactgagtttatattgtaaggaggcaagagagggactgtttccatgtgcaatattaactagcattattagaagctagtctggctgatgattccgaaataaccagcattctgtgaatggatccctgtgacctctgtcccacggttctaatgagcaacaggaccagctgcttctagtgttgccccatcagcagtaccacgaactgcaggcagagaaacacaactttagctggacatggaggggtcttgtcacttacaaaagacaacagaagacaccaatggaaattatttgcatttatctcattccccagttcttgctgtgagctccttttaaaaatgtttgaagtgatagatttaggggtttgagggacaaaatgttccagttggcctctgtgtcttgcatctagaatttgatcactgtgtaaaattgaaggcttatatttctgcacaagaggagaggaaaaggaagtggttgaaacactgaaactataggtagttgatagataaacaaattcagatatatataaatttagatagatagatagatagattagatagatagatagatagatagatagatagataggtaggtagatcatcggtcttgtgtgtgccttaccttttagagctaatccaagcactgatataataccaaagccaagtccttaattattccaggaacatcatagaaatgaaatgtagaaaactaataataatcatatactttgtatgtgtatgttttttcccagcacataaagggtttgtataagctagtggtcatcaagctgttaggaagaaaaagcaaaattaaaactaagatcttctcatttctattcaaaacctattgtctttttttctttttcatttttattttgcattatatacttaatgctgtaaaactaatgaggattcaatactagcagaaattcagacagaggggttcattattagttgattgttagtaatccttatctctgaagttattttcattaatttttcttttgtgtactttttcttagtttccagttttttaaaaaagtgtgttttgatgtataattaaaggaaacatcatgaaatgtctattatttttaaaaatactcatggaaaatgagtaacagcatcattgaatagttaacacatgacagcaaaacctgaatgaataattctcagctcggagacactgagaaatatttaatttttacatggtggagttttttctgttaagcgtaaagagtcacacatgctcttattcatccccctttatgccgagatattacgtcaacatgtgagaccatttcgtaaaccagtttgagatatttctggttactccttattatatagtttcttaaagcacagtgtagaagctggttagggattaattcctttccttcccatcctaacagtcatggctgacacctatataacaatgacaggttaacaagagaagagcataacaaatatgtttgatcacagttttacataacatgggacccttcagaataaagaccccagatacagggaaagcaatcctttttttctgagacagagtctcgctctgtcacccaggccggagtgcagaggtgcaatctcagctcactgcaacctctgccccccagattcaagtgattctcctgcctcagcccctcgagtagctgggattacagatgtccgccaccacgcccggctaatttttatgtttttagtagagactgggtttctcgatgtcggccaggctggtctcaaactcctgacctcaggtgatccacccatctcggcctcccaaagtgctgggattacaggtgtgagccaccacactcagccaaagcaatccatttttatgcttaggttcaatgaagtatgaacagctgtgtagaaatatgattagacacaaagggtatgatctaatgctaacaggctgagtggggaaactagcccatcctgcttgttcagatttttctcagcccctctgtgcagcactcactcctcccaggcaagaggtaggatccctctggaatgagggtctgaatttctttatggccaggtgttacacagaaaagctgggggtgggtagagtaaatgtttaggttttatggctggccttgaggaaaaggcgttctggtgtctgatctgacttggagaagagggattctagtgtctaaggtttgcctgagggaagaaagagaggagagacaggagggcagaaggtctgtttctgaggctgcttcttaggccttcaccttggggtgtcgtttactgagcctcaacaacagcatccttgtttctgtctaattttctctgggggttttctcacctttaccttgagaaagtttttgtggagttttgtgctttcttattcaaggccttcattttaaatattcccaacaaacaaagggatacagtaaaactcatgttggctaattagaaaaaggccagtatgaattaaaggaagactctatattacaaaatattgttttaaagtatagctgtgtttcttggaagttcatcattttacctgaatgtattatatctcattccaatgaatttgctttaaagacctgccagtggtggtttgtaattatagtattaattttttaaattcaattggggtcacgtaacaattttaaaatttgacacaaattctaatttgtttaattttcttactttaaattctttatataaagacaatgaaaaactaaattcgatagttgtacaaatctaaatgagaatataacttaagagttttcttaagtcattactaacactatggaagatctgatagaacacataagaatatactgagaccatcacagtgcctacacataataagtattcaatatatgtttgtcaggtgagaatatgagttgttataattcccagcattatattaagggaacaagtagatgtgtgaagagagtaccaatagcattttccattctcccaattgtcttcagttattatagcagataagtttttaatcctatgtttgtgctagcactagcatttacaggtttctccctgatatatggcccattaacacacaataatccgagttcagagtaattacttacatcattcaacagaatagatcactgaggcactgggtgaccagttgactttccatgtgatgagacatgatgagaaaacagcaaagctgaaggtgctacttggctcctctggaagccatatttctgctttctcctccctacatcactttctttttctttaatggaattctaggaagatgtgtgagtgggagaatctgagtgtggaaggtgttgcaaagatgtatatgttgtcaaaaattaaggtaaactatcataacacataaataaactatgttcctaaagctgcaaactttttaggtgagagcaattattcttatgctcaattatttgttaaccatcatgatgaaatgtcttccagatgcatctttgcttatatcagcttattaactaagagtagtcagattctatttataatgtaaaatattaaaaatacccaaatctattcagtccttcatagttcccaaagctttccacatctatcgtcacatttggccttctcaatagccctgtggagtagctagagatgatataattgcagctggaaatgaagacaatattcaaatggagcgtgtcaaccaatttactagtacaattttctcctatttctgagacacagatgtgaaaaccaaaaaaaaaaagaaaagcagaatgaatgccaagaaatagcatgtgattagaaatgttgttcatttcttctttcagtaaaccacagaaagataacaaaggctactgcgcccagtacagaggggaggtgtgtaatgcagtcctggcaaaagatgctcttgtttttctcaacacctcctatgcggaccctgaggaggcccaagagctactggtccacacggcctggaatgaactgaaagtagtgagcccagtctgccggccagctgctgaggctttgttgtgtaaccacatcttccaggagtgcagtcctggagtagtgcctactcctattcccatttgcaggtaaaatctcaaaaataagtcaaaggaaaaattccatttttctatctgcacaacagaaggagtttttgaaaaatgttgtaatatgcaacagtaaaaggaaaagctggttttcatccaaaataggaggttaatgcctgggtgcagtggctcatgcctgtaatcccagcacttcgggaggccgaagtgggtggatcacttgaggtcaggagtttaacaccagcctgtccaacatggtgaaaccccgtctctactaaacatacaaaaattagccaggtgtggtggtgcacttgtaatcccagctactcgggaggctgaggcaggagaatcacttgaactggggaggcagaggttgcagtgagccgagattgcaccactgcactccagcctgagtgttgagactctgtctcaaacaaacaaacaaataggaggtaattaatcagacaatatttctaccatataaatcactaaagataacctaaaatacaagtacttagaacctctagaaaatatcataaatggtgtaatattccattactcttgttattgttcacattattatcatatccataaatatcccagatttctatcagatgaaaggagaaatcttaaacatggacctcattagtaagccaagatagagaaaataaaaactgtacattatttgtcaataaatgagatcatgccaagcatctctgaatgttattttaaagtacattccatccaaaattctaagttctgatgctgttgcctcatttgggtggttataggaaggaattagattataaatagatattttgttaaactcccatactttttaaatattgaaattgataaatttatgttcaatagaattatcttcatctttaattacttcttatgtagaaaaaaaagaatctagtttttgagctaaatagaacaaagttcaaatactgttagttctgctttctcgatgtgtgtggtcttggctaaacttcagtttcctcatctgcacggtagaattgagaggattaaaggaaaaatcacactcatggaatatgtcagaaactagatagacattgttattcctaaattttctgttgaggaaaccccatgacaacaaccaacacagcacatttccacttctattttgataaaaccagaaactatttaaatattgcaataatttgaggacactctctatttagcattcaataacaacagtatggtcattaaattagagttttgtattcctcaatttactgcaaagtttgagggtttggagacactataagagtaatataatttgtttattcatctaagaagattgtattgtgatcaggcatgttatctaaaaatctctagggacttcagcattgacagaataagaaggggaaagagaaggagagaaaaagtaaaattactgcaaaaaaaaatgggaagagtgggtgaggggcagtaaaatttgttttaatagaacttaagaaatactgatgctatttcaactgtccttgcagttatttgaaaaaatagtgtctctgttaattatcacttctttagtttagatttctctttcttgggcttctttataggattctccaaaattgtttatttgacgtactgctcaacagtgaaaatccttttgctagaaagtagcacgtgtataataaacagaatgtaaccagcaaaataaagtgacagtagattctatcagcttttcctttagtactactaacataatttgtggtggccacgggatagttatcatacttacacagtgacactcaggcaaatcagctcaacaccatggatagatgcttgagagaaataataattagattagaaaaattctctgtgtgtgtgacagccattgtggttttctgattacaaaacccacaagactccattgtataaaaaaagaacacagtactttcattgctaaaacccagaaagttggtcactactgaggtgtgtgtttacttatatttacttatatctatatttctgtactctgaattctatttcatggatggtttctctatttttgcattagagactttgggtttaattactgtctttataattaatttcaatttgtgctacgaatttcgcaggttttcatcttttaaaaaattattgtttactcattatataattatttttattacatgttgacattggcaaaatgtttctaaatttcatatttaaaaataattatataagttatataatacaattatatattaacttaacaataattatcaataataattaataattattatattaataataaataatacttaatatgatataactaattataagctatataattatatattatagtatataacttatataattgatataatttacaattatatattaattatataacatataattaatatataattataattatagtttataataatataattatatattatatagctcactatataattatttttaaatataaaatttagaaacattttgccaatgtccacatgtaaataccatttagaatttgatcagaatgatgctaagtttatacaattaacttagggcacagagctatatttacattactgagtattctcatttaggagtatggtatgccattctttttatgtagatcttgtattatgcctgctagtaaaatttgatgatttctttcttacttttttttaattaaatttatgactaggtgtgttttatagtcattgctgttgttgcaagagtattaattataatatagttattacaggaggctagaatgtccataaaacatagagatgcaattaattcccttgttccttattttaataaaactgccattaatttctttagaatttgtacattttagagttgaactatgtaattttttggatcatattaatcagatttctaaaaatgtattcttttttttgaggtagttagttgatagaagtataatttatatacaagaaactgtgccccttttaaccatatagttcagagtttgacaaacgtatacaacaatgtaaccactaccccaatcaagacatagaatgttttcaccacccataaatttctcttatgctcatttccttcttttttttttttttttttttttgtccctgggttcaagcaattctcctgtctcagcctcccaagtagctgggactacaggcacctgtcaccaggtatttttgtatttttagtagagatggggtttcaccttgttggtcaggctggtctcgaactcctgacctcaggtgattcacctgcctcggtctcccaaagtgctgagattacaggcgtgagccaccgcgcccagccctcttatgctcatttctaatcaacttcccattatctctaaaggcatcgtcgttctgatttctctgacaactagttttgcttgttcctgaatctcatactcatgaaattgtataatatatactgctttgtgtctgacttcttttgttcaacatgtttttgatattcatccacattgctacatgtaacagtggtttgttccttttcattgcagcttttatttgtgtggttgagatggaaatagttatgtgaagaagtaggtagggtcaagctccccttcatgctgcctaagaattataaagcaattaatttgaacaactgcagtatcaaaaggaaagttagtatagaaatgttaggccactcaagatgacagactcatcagccacaacaaaaaagtcagataccagctttagacacaaaggatcaaccaaataatttattcctgcaatttgtagtttattccaattttgtttggtattatacctcctaattattgagaggaatcaagtctcatcaatcataaatcattaatccttgccaacaaatgtgtattattttccattttgttaacgagttgttactactatataataaagccatttcattttattatatttatatacaacaaccgctttacagtattctattagatttttgattgatagtctatggttcctctgatggaaaaatatggtctataaattattatatatagtttatatataatataaaactataaatcttaattagattctactctttcttaattgaaatttcagaaatttttactagaattaaaattaaattttatcttcatgtgctagcacaaaatacatttattaactttccagcatttatgtcccagatcagtttacatgcctgagccacagatgttctttctaatcttgaaaataattcacctttttgaaagaaaaaaatccctttcattgaaatgttaaatttattagaaaatcatagttatttcctaaatctatgtcattcacagtatacatttaaattttttttctaattcccattgttatttaaataagtagttttattgggaaaatgttattttttaaaatcctcatcatcaaatattttgttcccattgaagtaagaaaatataatctaaaaatttttacattttaatattttatagaggttttttctctgatatataataataaaaattggcatttataaagacttagtctcttccattaacatacaaaattcatatatttatgttcaatcctttctgctcttaaatgagtattagctatttggtccaatagtttaatagtgatgcgtcaaaattttcacaattgtgtttccaagaatgttttcttgcaattctgactcctgctttttgcaccaagaaatattaaactaaaattgaatttactgctgataactggtaagtttgatcttcatttttttgttatatcttttgaaaaagtctctacctttattcatcaaactagttcagaagttgtcaaccattttatattctacttctacttgcttgtgatatttttaaaagcgtgctatcactcatggttttctaattattaggttggtacaaaagtaattgtgttttttttccattaaaagtaatggcaagaaccgcaattacttttgccccaacctaacataagcagtcattcaatgatgtgtgcctcttttcatgaacgacatggaattttattgtctaacaaatatttattgatctccttctgggctccaagtactacactgagcatttgggatatattaatgaacataataaacaacttatatataactttagaaggtgttaaatgtcatggaaaaagaaaaaaggaatgctttaaggaagaccagaaataacaaacggagaggagaaagttgtaactttaaaaataaggtgttcagggtagacttaattaagttagcataattgtactgaatcttccacacttgttctttgaaactttacctgaaattatcaaccgtatttgtattgttcaattatattttatttgcatatgctcaatttttggaacattattcttagcttttacatttgatttcataatcatgtataagtagatacttggaggagcttatactatgttacttatcattgatactcattgtcattcatttcaaataaaatatttctttttcttaaattcctaatttaaattcacttcttgttcattcatctcatctttactcatctctcattcagctggagtaggtgtttttcacatgtaaaaaaaaattctgtcattgtacatatgtgtttttgactgggtatttaagtctgtgttaacacaagtgttagcttttgcgttagttctcctcacttgtgttacttatcctcaaaactgtggactctgaccaattattttctggccatgttgtaataaagaggaagaagaaagcaatcctgacttttgttcctttgtaaggaacattttcctctctccagattgtagaaagattatttctttaattgttgagattcaaaatatttgtaaaattcatttggggatagatttattctcattgttttctttcatgtaatataaataatataaaaataatgggtaatatgccctaaacattgtgcccttttcatcttaaaaatgttttcttataccacatctttgattattttctattttttatgtgctctggttctcttgctaagaaacactattatttgattttgaacttcttacttgctccaccttatcagttatttcacacattattttcatctgtttagcccttcttgcattcagttaggtcctttctttctgcattcaattgaatatttaattcaaatttgtttatttcttgcagtgccatttgttttctattttcaatgaagattgttaagtgttggttttattttttgtggtttctattttctgtagttacttccatgttcaaattatcctactttttagtctatagttcatcttgatttgttttttcttgtgaaaaactctgctatagagggcatgctttagtgcatggcaatgaaaacaaagcaaaccaaaagccattcattaatgtgttcttctgtttagaataaaaaaatttcccaggtatttgcttcttctgcctaatgtggagtatggagttgttttcatatgtcccatattgatttacactttaactatgctgtctgcgtgcaaaaaaatgtgcttatacaacccaggggtggaaccaagctaagcaactctcctcaggtcctttacctgctcagaagtcttgaaagccccattatactcccttctcccttacgcacaccaagaattataaatcccattgttttcttcatataagaatatatttgcatctcatcacattcatatgcacagccttttattctatgcatactcaattagcacatattggcaagttaggaaagtaggagtgtgggaataagtatgaaaaaagacaccattgtggcatatatatacacacacacacacacacacacacactcttacaatttcctcctctctgaatccttcagatactgaataaaggacatctgctattattatgtttaatttatttaattatctttaatttcctcttattattaaccccaaataccttaatttgaaattaaccaggtttttcctatgcagtttgtcctattttctatcaggcttaaacaaatactaatctgcaaaattccaagtagtccaattagtgtttcttcagccccttcactcataatacaacatgtagatgaacataacagcttcatattcaaacagataatactttggctttctcagatggaagcacgcagagcaatgggacaggattccgtgcctcaaagtaagggctgagtaaggacccagatgccctcctataagctgccagggaggtggtggtgttctgtttcagaggacaaagcacacagaggcccagactgtgttcctcgagccctacccattagccccaagatcatagaaaaatcttccaagctggaaactggttgacaatgatcctgactttcagtaggtgtgttttttaaactctgtatacctttttcctctacctgtgttgttatttgaatatgaagttgggagaagacatatcagagattcccaatctaatacgttttaaatttgacgtttttttcccttagactagcattaattgtgtttcacaacaaaacgagatagcatttcatctcacgggacagtacactagatttcatgaggtctcatattatttacagtaaaaagtttctttcataaacgcgcttttttcaagaacaatttaccatgatgatgtcttgaaaacacagaatttaggctctgccacaaatttgctttaacatgtaatgattcatcaagttttgttctccatatactattttagagagtactgcttggcagtaaaggagctcttctgcgcaaaagaatggctggtaatggaagagaagacccacagaggactctacagatccgagatgcatttgctgtccgtgccagaatgcagcaagcttcccagcatgcattgggaccccacggcctgtgccagactgccacatctaggtaacacagagttctcccaagactttggaggttaagagaaatttactattttgaagaaactaaggtgtgaacttaaagcttctaatatttctaggcatttctaattttttttttttgagttctgccttccttagatacctactagcggaatcctaatgctatcctaattgtacagattagcgtaaagatttatttcatttgctctaaaataaatgaaacagcacttttaattactgcttgacattttagattctttcatagggaatcttatgagagcttgtaacattttctggagtgtgtagaaaaatggctaagaaacatcttatattttgtcagcagagtggaataaaggaataaagtcctttttcttagccaagaaagcatgctaaaataagacacatgtttgaaaatgctgatctcacaaaacaaaattgaattgcttcatgttgctttaatgaactttgtttgtaaggaactatgttgttatccctaacagctggtttaatttcaaagacccaattctttcacattcgaatgcaaagaatgttttccagttgtatattaaaaagctgagagagaacttgcatttcttagcactcactctctcacagaatatgtgagatatctggatgctcacttaaaacaaatttttatcctttccccttcagattataacaaagaaaacctaaaaagtaagtaattgtgtttgtgcccttgaaaccttatgtgtatgtaattggattcatgcatgtgtgtttacacttatatttcctgatgcccagaacagaatgtacagccgctctcaaagtcttctcaccatactcatccagggtataacatgagggaaaatcatggaaggtctcagtttataacttttaattagaccgtagagagtaccatatttagtgagtcatacaaatgctatctcccacctcaaggtttttagtctgttttatttcttatatggctataagtaagccaaatttaaaatgtcaccttttgattttatattcttgattcagtataatctaattatgtatagatgaaacccaacatttatttcttcattgagccaattcatgatcttcatttgtgacaatagatccctcttttggaagttaggtatttttttaagacagctgatttccagtataaagtttataggatcaaatcaaatggctttaaaaaggtcatcacaaaaatctctgggaaagaattcaagaaggttgcttgaaaagggatgtcgtacaaaataacttgtatcagatagtgctttaacctgtcagaatgcagaaatcctcaggatgaaattagaaaagcttttcatgtcaactaacttcctctggtctcagcgttctaaggcttaaatcacacttacttcctgtaatacatattccagccctaatctcctctgcatttgactaggtgccaggacttgattttaccagcttgcttaaaggaccaacacttaaaagtattaaaagcaagaatgctactggtcctagttgaattactcagaggtattaaacaagaagagttatggaaaccttaaaaatctattattctggtagcctgtcaaattgttattcccaaatcgaaggaataaaaaaaaatttcaattctttgttatatgagcagccaaagtttactttgattggactttgaattgattactgtgctctttgttatccatcgattgctcactcatccattcaaacattcattgagggtttgctgtgtggaaggaccagaccattcatcttaaattagacaggaacaatacacaaaaaagcatccacatttctgcctttcacactaattataaaggaattttgagcagcacaaactagctacaataataaattttctagtctttaaaaaacagctttatgaaatcttcgatacatgtaagaaaatgagctcctaatgtcatgaactaaccatttaaactgaaagtagggcagggaatatctgaaagtatgctccttgagttaaggcaattattattaaaagagttggctttagcaggtcaatagtggagcaaagaaaacatccttattaggcagtgtcccatattttataggggtgtgtaaagaccttaagtattttccaaagagtgtcttgatttctgagtaaactctaaagaatatcaccagctcatttggaggggtcacaaaagagctgttaatcatttgtttatctttgcattattttcttcttccttgagcatagggactagaatttcagtagtttttcttttttataatttctttggctgaagtgaaacatctttttgagcaggcaatgcctttgtgctatttacattatttgtaagcaaggattacactattcctctcttccgtgagacttcacatactttattacagaaacactcagcagtgaatctgtcttctatttcagagcttctgtggagaaaaggttaatttgtcttaatcattgaaataattacttttctcttgaattctctttttccttccatctttgctctttctatacatatacatggagaataaaggacccaatgaatgtacccaaatccaaactgactagtgtgatacatacattatctctcttaatactcccaatagttctacaaaattaagtactatgaacgtcctccattttacttacaacccaacatgagaaagagagattaagcaatgtgtccaattttacccatctagtaaggtgataagccagaatttgaactgaaatctatctgattctggctctgtcttcagagccagaatgttatacctgatgcatttctgttattactaatgtttcttaaaaaattccaagacctcatgtgtaataaagcttaactgattgtttaactgattcattcttgaataaattccactaccttttcacatttgttataattgattattcttaagaagctctaattgataaaaattttattgttcacaattttaaatcacccaggccacttgatgatttgactttttgttaacatttaaatgaatacatgctttgtgtcccagaccatttttccataatgatcagctactattttgcccacgaatgaaatcatgaagcgtcagtgtctctgtgtgtgtgtgtgtgtgtgtgtgtctgtgtaaaactcatgaattatttcataatttgaaatgtggtttgggtaatttttgtggtaatattaaagttacaagttcttagaaaattggagtttaaagtaactttaacagttatttaatctaaagtctcccacaatgaaagaatcttttctagagcatctctgacagaaacattccattttccctcctgaatttatttctcaattcctttgtagaatacattaattatttcaacaatttagctatgtagaactaaattctggttctctagaactgtcaacaactgatcttcactctatttctttgatttatttataatctgtatgatagtccttaaaatatttggagacaggcactcaatctctctgtagtcttctcttcttcacattaaatatctccaatttttaactttttcttttgcaactgacttccaaaatcctcacctccatgtttactttttactggctgccacaagcttgagaatatccagaactgaacacaaagcacagaacatgatctgatcagaattgcaatgcaaccatattttctgtgataaggccttgttcttttatgagtatagattaaactcatgctagctgtctaataatttcatgacattaatagcccttctatacacatatgggtttactgatttccttgattttctaataaattttctactagaccaatccaattcataattatttttattacattcttctagcatttataataatttgtactttacatgtttagttactatgttctatgaggcatagtgatccaagacacttttctttttatatgaagctctttaccaatgtaaaatgtcactcttgtcttgttttagtgcttttagctttaaatttctagtattaataatggtaattttttccttgtatttatgttaacctagcacctctttgttcctcttttatttttcttttcttataaacaacatatacctgggtttactccaatcagatagccattttctattaataggaggatttagcctgttcatattttatatcacaactgatatactctattttatccctactatcttaactgtatattttctattcgctttacgtttttgcttcttctttttctttttctttacttgttactggttatgttttctccactgctaagtgaaagtcagacaccaattctttctacacatcgcagtgctctatttattcttcatttctgcccaccccatgggactttcagaatctccaggagatcctcagactgcttggatcttcttccttacctcccctggtactcttaggtttctctttgcagagtgattctcaatattgagtcttacctagcgaattagccaagactccacccatcacagccccttcacatgtatcctgaagttattttctgattcaattgttattaagagtctcctcatgtatttccttagaagggaaagtgtcttgggagacagaatccctgaagccttgtgtgttatcaaattgttttcttttatttgggtagttgaaaagtgtattaactgggtatggaactcctgagtgacatttctttgggtggtttatagatgtgatttcactcttttctagcttctagtgttggaagcaggtagtctggtgtgaatttatttttttcctgtaagtaatttattctttctatctaggcagaaactttttgatttttccctttatccgtgatatctgtatgataaaaattgtttaatcatgtgctattgaacatctctttccaactccatgatagggcttcatgttgggagcttgagataagctatgatgggaatctttatcccattgaaatcaggatggtagacagacctttctaatgcaaaaagttgatcatgctattccacctcgtaaaacactcctataagaccctccatttgctgcagggcatagtccaaattacttaaattggtatacaaggacttttatcactaaacctgatatctgcagctttacctatagatactccccaactccaacaatgaaacttctaggtgcacttttagaaaggcatcacatctgcttagttcagatcttggtatatgctattacttctttatagaatactatctctctaccctgactccattctactctcttaattcccccatcccacacacacgtttgtgtgtgtttttttgttttgttttgttttgttttgttttgttttgtttgagacggagtctcactctgtcgcccaggctggagtgcagtggcacaatcttggttcactgcaagctccgcctcccgggttcacgccatcctcttgcctcagcctcccgagtagctgggactacaggcgcccaccaccatgcccagctaattttttgtatttttagtagagatggggattcaccgtgttagccaggatggtctcgatctcctgacctcgtgattcacccgccttagcctcccaaagtgctgggattacaggcgtgagctactgctcccagcccacaccttttccttagtctgttcaggttgctataacaaaatgccacagactgggtggcttatacacagaaatttatttctcacagttctggagtccaagaagtccaagatcaaggtgccagcagatttggtatctggtgaggacccattcctcatagacatagctgtcttctccctgtgtcctcacatggcagaacgggcgagggagctttcttggacctcttttataagggcattaattccatttatcaggactctacccttattcatttcaacacatgaatgtgggggaaagacacaaagactcagatcatcataaccttcaactggttactggctactcaccatttatgtttaacttacagttactcactctagaaaaccttctcagtatgtctaaacctgagttagtgccagccctgaacgagactcacaagcatcgtgttcttactactcctataatgcttcttatattttgttttatttctccatttattcacttgtgtctcatgacaagcaaagacttctcatgttcaccacagtgttctggatacctaacagtcactattgatttagtgaatgcatttacatatggatgaatgagtgtataaactcccgtagtgttcagaggaaaagaatcaagacagaaaattggtgagagaaaagataaaaaggaaaaagaatgacgattcaaagtgatcctttagacccaaagagatcattgatgtagttcaagggaagatcagatcaaatcagaagcagtcatcaaggaataacaaaacaaaactatcttgaaggaaagaaacatttaaatccataaagcaaaattattgttccaactatgtcatttcttgcattttaaaaaaactcactaaatcatcatagatttttcttttaacatgacagtaaattctactttctgattctttttagaaaaactaaccttatatttaccagtgaaattacactaccaactttactaattacattatattccctcacatgttatatatggtgtgagatgctctgtaaactccaaatacaaaaatagttatttctccctatcttttaataattgttccaactttattattagcagaaagcaaagctcgtcaatttaagccaccccctaccaccaccacccacccatcttagatcgagaaaacttgggatgactcagtccaaagagaatcagactagctttcaagtcagtcagagaggaacctggtcagtctgttgagtccaccactcctgtggtgccggatgaggcatgctgtcactgatcttgcgcagatgttttattgctgtgctgatgttcttccagttccaggtgcctgcctgagtcacagtggtgaccaggcctggcatccgcaggcttcagaatcacttgcttagccattggtactcagggcataatcactgggtccacttcatgtatgactaagaagtcatacatgagcgtctcttgtagcctccaattttgcaagtgccctatttcaaatgctttaggcatctactgccataaaggttttattttgactttgtaatatgttatattaatatgtccccttctattaaatcattattctctgactatgatgtagtcagttgctaatatttcgtttaggatcttctaaacttaggttgtataatctatgtatgctcttttcttctgtgctaacattgccaggttgtgatattggtattataataatttctaaaataatttgaaagaattttctcttactggaacatattataaatatggaaattttatctctttcaagactaaaagaatgtcttttataatgtgatgacttaaaaatctagtttcctgcctatggtaatatttatattttctaatgcttcaaatttggggattaatatgttcctatcattttccttaaggtgtgaaatataataattacacattattttaattaactttgaatctgttataacttttttatcatttctaattttctgtttgtaccttttctctttaattaaaattccattgattagactactatatgaatatttataaaaccaactattgatcttttaattctatcatttgtgttacgtgtattctaacttattaacttctgcttgtgtatggctgttactttattacctattatcttttgttttctgtagttattgtttcttacccaaagtcttgggttaaatgttcagttcatttactttcattcttttttatctagtggaaggagataacactatgactttatctttgagcacaactttggctaaataaaatacacactattaagaggcttgtgtctaatctataactatagttactctctagtttttgatcaaaaagtcattgatttttttctttttaaaaatgtcccattggattttatttaatctattattaattgcactttggccattaagtagatatgcataatttctgcctttaaaatttattgagattaatccacaaatatttgaaaggaaggtttattctctatttttgggtacaaaggtcattcttagctattaaaacattgttgctgattattttaaaatactctgtatccctgtttttgtctttactcaatctgttaagaggatttaaatgtgattaataaggttcttttaatacatatacaatggactttgtactctacaaatagtggtatgagtatggcatcttttggtttccaacagtaatgctgattttactttatacatatcacatcggccttttaaattgaagtttgacaggagttcagtttttagatgaattaacttctcatttgtctcctaatattttcatttttcatcttctttcatgatttttcttctgtttgtaaactgacagtaaatgtatataaaagctcagctctagacaatcctgatgtcaagtgtaattatgtgaaaatattttatttgataatttccatgatttatgtcaggtcatagataatatgattcagtataaatggcaccagcccaagatctacatttatctcagaaaaaaaaaacctatttcctctctctgagtctcatctttaaaattctattattctataagctggatttacttacaactagaccttactgaactttataaatatataaccagggagaacctgatacataaatatattattatcatgaaatttattttactgattgcttaaatacaaaaaattgcttcttaaaggtttgagaatgaatgaaataattattctttacttacagcattcccaccaatgacgtcctcaaagccaagtgtggacattccaaatctgccttcctcctcctcttcttccttctctgtctcacctacatactccatgactgtaataatctccatcatgtccagctttgcaatatttgtgcttcttaccataactactctctattgctgccgaagaagaaaacaatggaaaaataagaaaaggtgagattctagtttcagctaaccatgtgtagtaatattttaaagccttgtgttttatggagttggtatgacgtgaggaatatatatgacactgcaactctctaaatatatccgtagccctgctttaaaattaagcacccccatcatgatctttacaaatgtcaatgactttttggcatgggatcaaatcaaatgccctagggttactgtttcacaaaaggtaaaatctaaacaataatttggaggaaaatggctattcctgctccttgtcttctcctcaggcctgttgccattgctggcttggagagctaaacatttctgagaaaatgtgccttttgactctagaccaaacaaaatattttattgctctcaaaacgaaggatctttggctgccttagtataacttgttttcattactcttaaatgccatatttcttagaatctaagtctaagtacaaagatctaacctgcttctgagctcattcctgatcttgcctgtcttgcctgcagagaatcagcagcagtaaccctcaccacactgccttctgagctcttactagatagacttcatcccaaccccatgtaccagaggatgccgctccttctgaaccccaaattgctcagcctggagtatccaaggaataacattgaatatgtgagagacatcggagagggagcgtttggaagggtgtttcaagcaaggtaaagttacctatggaaaaaaaaactccattgaaatatgttatgtctgaaaagctaccaaactattagcttggtatatatataatattagctttatatatatatacacacacatatatatatatatatcaagctaatgatatagtacatatatatgtacactgtgtgtgtatatatatatacacacacacatatacatatatacacacagtgtaatatacagtattatatagtattaatacaatattatatatgtattatatggtaataatatactatataaaatagtttttatcaagttagttaattctggccaggtgcggtggctcgtgcctgtaatcccagcactttgggaggctgaggcgggaggatcaccttagtcaggaattcaagaccagcctggccaacatggcgaaaccccgtctctactaaaaatacaaaattgagccaggcatggtggtgcacaactgtagtcctagctactcgggagtctgaggcagaagaattgcttgaagccggaagacagaggttgcagtgagccaagatcgcgccactgcactccagcctgggtgacagagcaagactctgtctcaaaaaaaaaaaaaaaaagaaaagaaaaactagttaattctttacatcattaaccttatttgtattgcataatacacttcaaatcttaatttcccctctttgctggaaaacagtagcctatatggagataatatattttactgcacttaaaataggtaatttattggagatgtaaatgtgtcttcacttctacgatttttaaattataagaaattagtaatatagttcaataatatttccttttcaggaaagaatttaccatatatttttaatcataatttcttacattgttttaaataactccttacattataatataatgtatagatattaagtagatgttcatgaaatgacaaacagaaaaggaataaaaaggcaaaaatttataaaaaatgagctcaagaaaggagtatgtattagggccaatattagcctatgtatcacctttgtgcgaattggaataaaataccctttctgctgcatggatatggctccacagacacacagctgagccagagaatatgaatttggttttaatctccaactggcacttcggcctggtgctcttgggcaagacacaacttaatctccacccatggtggccctggcgtagaggttacatgaatagtgaaaatctcacacaaacatgtcttggaaaccaaaatttgcaaggcaccactttaggctcaatggagggtacaaagatgagtaagccatagttcctacgtcatggaatttagattctggacaaagattaaaaggttaggatgtggctgggcgcgatggctcacacctgtaatccccgcagtttgggaggccgaggtggtcatatcacgaggtcaggagattgagaccatcctggctaacacagtgaaaccctgtctctactaaaaatacaaaaaattagccaggtgtggtggcgggtgcctgtagacccagctactcaggaggctgaggcaggagaatggtgtgaacccagtatgtggagcttgcagtgagcctagatcatgccactgcactccagcctggatgacagagtgagactctgtctcaaaaaaaaaaaaaaaaaaaaaggttaggatggcaatgataatcctattgggggtgtatacttcagaattcctataatttgtaaataatgcatctttcaaacttcattgtttgaaattgttgaaaataacaccagtgatttttattagttgacatcaaggagttttaagtatgttgtctttctttgattctgtggctctgggaaaggctgcgtgagacttaaccagccttttacacaggggaggtgggaggatatgtataaatgtgggtaggtataaagatatgatgtccatgatctttggtttctttttcaataaatgtatctctcagggcaccaggcttacttccctatgaacctttcactatggtggcagtaaagatgctcaaagaagaagcctcggcagatatgcaagcggactttcagagggaggcagccctcatggcagaatttgacaaccctaacattgtgaagctattaggtatgaaaatacaagtgaggatatgtatttcatcagaaaacagaggctttccaagtttttctccccttgttcgtgctttttccttttctccttgatcagtgagacgtttcagttccttttcaaatttagcttcttcaccttggtctatcccaaccctataggacattcatggcttaatggattatccctctttaaattcatcacttcaatttgtctctgtctttgctgcctcctatttataaaaatgtttaaaatgtccccattgtaaaaaattaatactgcttctccatcacaaaatttatcaatattgaaaagtctatatattttacttattttgtttctcctctttcttccctaactagaatgtcatgagcagcgatttgtatcttttctgttcattgttgagttccagtttattcccagattcataaattcaacacaaattttacaaatatttagtttgtattaattacacagcaattaataaaacttttcaaaaatctgtgctctcggctgggagcagtggctcatgcctataatctcagcactttaggaggccgaggcaggcagatcacttgaggtcaggagtttgacaccagcctggcgaacaaggtgaaatcccatctctactaaaaatagaaaaattagctgggtgtggtggtgtgggcctgtaatcctagctactcgggaggctgaggcatgagaatcgcttacaccgggggggcagaggttgcagtaagccaagattgcaccactgcaccacagcctgggtgacagagtgagattccatctttaaaaaaggaagaaaaaaaaaatctgtgctctcatagagcatacattcactctatatgaggagacagataataaaaaaaaaacaaaaatatgggatgctggatattaagttctacagactttttaaagtgttaaagcagagaagttggaatgtgagctacaaagtttgaagacttcagtgaaaatattacatttatataaagacctggcagaggcaagggaataacagtgtggatatcttgggaaagaacattccaaaaagaatgagaagcatttgcaaaggtactgtggcaagagcagatgtggcatgcttgaggaatagcaaggagatgaatgtggctggagcagagtgaaagatggggagagttatcaaagatgagatcagagaggtaatgggggaggtaggcaggtcctgtcaggccttattaaaaacattggcttttaatctaagttgggaaacaaatggagagtttcagccaagacatgacatgacacaactcacattttttcagatcttctctggctgctttcttgagcattgcatgaagggaagcaatgaaatcaggaagacagtaaacaggtattgcaatgatgcagcaagagaagatgatggcatgggccaggtggtggtagtgaagatcgtgagaagtgattctcttccatgtgtaatgtgaaagtagagccaaataatttgctgacagaccaaatatgagatgtgcaaaaaagggaaaagtcaaagatagtatcgaagtatttggctgtgcagatgagtgaatggaggtgccattggctgagttgaggaatactcagaaggaacaggttcctttagtggagagagtgggaattcatttaaagatgtggtttttgtccaggcactgtggctcacgcctgtaatttcaacactttgtgaggccgaggcgagcagatcacctgaggtcaggaattcgagactagcctggccaacatggtgaaaccccatctctactaaaaatacaaaaattagccgggtgtggtggcacacgcttgtaatcccagctgcttgagaggctgaagcaggagaattgcttgaacctgggaggcggaggttgcagtgagccgagattgtgccattgcactccagcctgtgtgacagagtgagactccatctgggaaaaaaaaaagacgtgttttttggcgatgcttattcaacatccacatgatccacatgaagttaaggatgcatgtggatatggaggcctagagttctggtaaggatgatctggttagacatgaatccaaggctattgcagtacacagagtatttaaagccatgaggttgagtaagacacctaagtatggagaaagcaagccagaacagagaagaggtccaaggaccaaggcctggagcattccagtatttagtgtgagaaagaggagatgaacaagaaaaacaaactaaaaacgagcaaccaatgaaatagaagtaaatcaagagagtgtggtgtcctggatgtcaaatgaagtattttaaataggtgaaagtgagcatgtcatatgcattaagtcaagtgagaggaggactataaagtgaccttgaatttgtaaacatggatgttcactggtaaccccgagaagagaggtttcttaggaatgttgggggcagaggaaatgtaagtgagctcaaaagaagaggaagagaaaatctggagaaaaagtataaacaacttgttttgccataaaggtgaaccaaggaacatggatagagctgaagggggcagaaagaagcatgcttcctttcatgtggaagtaatagcacatttgtaaattaatgagaatgatcccgtaaagagaaaaattgatgacagggttggcaaatagtggaagtaacatctttgaacagtgagagggaatggaatcttggcactgttggatggattggcctttgtaaagagaatgctcagatcaaccatagtaataagagagaattggctggagattgtagccagtctcttctgattgatttagttttctctgtgaggtaggaatcaagatctcatctgagggtgaggaatgggagaagagttgaagatttgaggaaagagaagaaataaattgtcatctaagaaaatccatttttacataaaatataaaatggactagagaaacataatatgattggcaagaacattaaaagcttacttgaaattagagattacacatttaacatgagactggtcagcatggttgtgtatttatccaaatgctgccagctgggcatagactcgttaagaggagttaaattttaccagaattagcattttgccacacaagtaagttgaagtcaaattgaggcaaataagagaggtgtatgcaaaggagtgattattatgattgatcaaggaacttaagctgtttaaggaaggaagacaaaacgtgaagaaagtagggatcattaaaagggggtaggatcaatagattaaaagtttcagggggaggagccaagatggccgaataggaacagctccggtctacagctcccagcgtgagcgacgcagaagacgggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggacgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagaccaaactgcaaggcggcaacggggctgggggaggggcgcccgccattgcccaggcttgcttaggtaaacaaagcagccaggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacagcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaatgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgctgagagattttgtcaccaccaggcctgccctaaaagagctcctgaaggaagcactaaacatggaaaggaacaaccggtaccagccgctgcaaaatcatgccaaaatgtaaagaccatcgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcatgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctgagtgacctacaaagagacttagactcccacacattaataatgggagactttaacaccccactgtcaacattagacagatcaacaagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccactcaactacatggaaactgaacaacctgctcctgaatgactactgggtatagaacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagctgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaattggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgagtgaacttccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcatggtactggtaccaaaacagagatatagatcaatggaagagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttagacctaaaaccataaaaaccctagaagaaaacctaggcattaccattcaggacataggcgtgggcaaggacttcatgtccaaaacaccaaaagcaatggcaacaaaagccaaaattgacaaatgggatctaattaaactcaagagcttctgcacagcaaaagaaactaccatcagagtgaacaggcaacctacaaaatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcaggggaggggggagggatagcattgggagatatacctaatgctagatgacacgttagtgggtgcagcgcaccagcatggcacatgtatacatatgtaactaacctgcacaatgtgcacatgtaccctaaaacttagagtataacaaaaaaataaaaaaataaaaaataaaaaataaatgcatgaatacccaaaaaaaaaaaaaaaaaaaaaaaaaagaaggcatttcagcatgcaaacaagtaatacttttaggaattacactgacttcattgtaatgcataggaaaaataaataattcacaaataaaaaacaaacaaacaaaaaaaaagtttcagtggggtcaaaggatttttagagttagggcagtcgaggcagtgaactagggtgaaaaagggaggaagtgatgaacaacaggtagagtaactgaaaatgagattatgggaggtatacaaatgtatggaatgacaaagtcaggggtatgacaatgtgagtgattagctgaagttgggcagaggtctaaggaaaggaagtaaagtaatttagtggtcaggatattggaaggaatctagataaatattaaaagcaagaatttctatagtagtatctcgaatagtttctggcacattgtaggcactctataaatatttgttcaataaatgaataaacgatattaagtacataacaattcatcttgaaaacacttcttccatttcctcaactcctaaacattcatttattcattcttcatcacacaaaaatctgaatctggcttctgccccctaccctccttatttactttctcaaatagtatacatttagtcattaaacaaacatacatacatgcatattttcaagattatcaagcaattcctgtttcaacttttcttaaaatgttgactctgtattcgtatacagtttataacatcctgaattcctgaaattgattcaaacagaaactatgtacagttttccaatttaaccacagtagcatgcttatacatcactacttggagttttctctcatttttattgtccaacaagaactgaaacttatgttgccgtttgttaaatgctatatctaaattccatttggatatccaatgaacacaggttctattttttatcaagtatacataagcagtaatcattcatgcaagtgattccactactctcatagtttcttgtaagttaatcccagtgaaataagatgacaatagtggaagaaaggggaattgggaaaatccataaaattcacttctatcaataaaagaaatctactttgttttatatatttgatacatcagccacttattgaacatgttcgtatcttaaaaattgtttcccttttattgaaatcctagtggataatggttttgtagtcataaattgggaaaaatggtataactaaagaaatatgcagaattttagaatgggagtaggcattgaatattatagactcttacggaatattctggcagtggagcaaacagtagaacaatcaggtgagattattcattgaagatgtcatgctaaagtttaaagacatggcttgggctaggcatgaggttttcttatgcccatccatctatgcatccatccttaaatctatccatgcatccacctatgcattaattcatccatccacccatcaacatatattgaaaacccatagtgagccatgtgtagtattagacctgaggttacaaggatgaataaaatacagtcttcctctcaataattattatattttgaagtcccacctaagagttgggttggagtaattacaatatagaaaacattttgaggtcttaaataaatgtatatacatatataattgtaaattatatctatacacatgtatatataattgttcataataatcacaaagtagaaatcttttgtatagtattttctttatcagagtggaaaatttcattaaatcaatttagttcaaaactttatttctcagagaaatattttgtcatttttttccagtataagaaattatttttaaatcaactcaaccagcataacaatggtaaagccagttgatgagataggtaagtccctgttaactaaagattatccaaaaaggatcccacagaagaaacttctaagtattatccagttggtatttctttattttttccctttagtttacagaaatgtgctaattgaggtccttggaattaaaaattaatgccaaaataaataagccaagatagcagtattcctcatttctcctctttttcaaacagatacttttaacttttgcctgaaaaaacccaatagatctaataatctatttttaagatgatgtgaaattatgcccatcttatctaatattccaagttctccagtttccctctcaaatatatcaagactttaagatttgaaaatgatcttctggctttaaatctaagactttatttatattaacttttttggtagcatgaacattgtaaaacctaaatttcaaaatgttttactgcagcaacttaaatacttcatctaagacacttgaattgtatttgttattgtttccaaacttgatacatctttcttattaacatccttcatagaagtaaacagaaacagacagcaattacattacatactcagagcaagcatccattcataaattaactcaccaaacattatgaggcatctgctctgccagacacgtggtaggtgtggggaatttttaaagaagaataaggttcaatctctcctttcaaagtgctaaaacgttggggagaaacctcaaatagtcaaatgattataaaataggatggtaggttctctgtgaaaggatgacagtaggaaaatctaacagttactggaaggatcaggagctgaggcaccctagggagagtgataaactgaatcttaaaggcaaagaggagatgtttaggcagagaaatagggtcagggttcaaccatggtgttgtcaacaaaaaaccagggcctccagatttcagagagaagagccactccagagaactgatacaccatgtacttatatgcaccatgaacttgatgcaccatgtacgatgtacttatgacaagtgcccaggacaacagcgctctgctggaagtcctcaaaaactttagccaagggacagaaaccccactacccagagtgcatttcctagctgagactccttttgtgttgtgattaaaaggatacaaatattaaaaaacaaacatacgacaacaacaacaaaaaacagggcttcatatgttctgacatggtcgtttgcttttggagtgctctttccattgtttcattgtagaaactgagactaacagggatggtcttttggttccaggagtgtgtgctgtcgggaagccaatgtgcctgctctttgaatacatggcctatggtgacctcaatgagttcctccgcagcatgtcccctcacaccgtgtgcagcctcagtcacagtgacttgtctatgagggctcaggtctccagccctgggcccccacccctctcctgtgctgagcagctttgcattgccaggcaggtggcagctggcatggcttacctctcagaacgtaagtttgttcaccgagatttagccaccaggaactgcctggtgggcgagaacatggtggtgaaaattgccgactttggcctctccaggaacatctactcagcagactactacaaagctaatgaaaacgacgctatccctatccgttggatgccaccagagtccattttttataaccgctacactacagagtctgatgtgtgggcctatggcgtggtcctctgggagatcttctcctatggcctgcagccctactatgggatggcccatgaggaggtcatttactacgtgcgagatggcaacatcctctcctgccctgagaactgccccgtggagctgtacaatctcatgcgtctatgttggagcaagctgcctgcagacagacccagtttcaccagtattcaccgaattctggaacgcatgtgtgagagggcagagggaactgtgagtgtctaaggttgaagacgttcaaataaaatgctgcagtttcctctcagactctgtgagccaggggaatcctacaccagaggcccaacaagacccacataggaaagagtaagagtaaacatgagtagtgtgttgtttgcttcccagggagagcaaagacagtgcaaaacccatgtggtagacggacccattgaaagccagtgattggaaacacaggctaggaaatgtgtcagataatggagacaactattcttcttcatgaaggtttgtaatacacactgcacagggaagaatgtcatcctgttcagtttccaaagtagctggtcacagagtaaagctctgctgtattaaattttaatatgaaatgggattcagagcttcattttttaattgagttaattcctgtccattgtgaaagtagcttaatcgtcatgtaagacacttaggtgaagtacagaaaactaaaaagaaggaaaagccactcattctgtctccccccaaagacgagtcttagtgttttattaaatctttcttgcatttaatgcataattttatatttgtttgtattctgcagagttgtggttacattttattgatagagggtttctaaggaatgaaacagcaaatcagcaagcctgtaactctgagttcccttttctgtcctgtgcccctccttttttcctttctgtgcatttgtcatgaattaagtctgcttattttattcctccagttttcttgaaacaagtttttgtatcttactgatttaccttttcctttaaaaattgtattggtatcttttccattgctttaatctttatttcttcttctttccaggacttctgtccttttttagttattaagctatttttttcttttatttcattcatgtaataaacactctggcagaaataataacattttaaataccattgtagattgatatgtatatgccttttgcaaagtattactatttttcctgcatttgcctttgaagccacacatttcaacattctcacctattaatattattactagagtctatttaaatctcttaaaccataatttataatatgagatttcacaagatacacttgtggctctgagttatttcaaaactgatatttctgtgcattctgacattttattccttttagaaacaaattcagttggctgtagaattaatatcgttctgcttaacaactttgtgcatgacatattaactgattttgttgtatttcatgatttaaaaaaaaaagcagcagctctggaactcagattcctgggggttcagattttttctcagtggatcctcacacaatacatctttatctaagtccatacatgatctgagtttatttctgatatagcaggaacccaaattctcctgaaaacgaacagggaagagaaaataacagtctactctataccagcttatcttaattcagactaattggtagttagggcattggaggaatggtgtgaattcttgaaagtagattttcgctgtctgctggaaaactctgcctgaaatccccaaatctgcctcatgtaacagcttgacatctttggtgggaaacctgccatttcttattcatagtgtctccaggcaaccaagacaaaaatgtagaaattttcctagactttgccctctctttcatcatcagttttctattagttttacttcctaaaatcctcctgctgacatcctcttcccttcaacccaagagccaatgactcaacctctaccgttgcccaccctgtctattagctggccttcctctcatttcagacctgaacagattctgtaggatacaacgacagtaaggcggtccttagtcacagttctggttcataaatagcgaggtcagcatttaggactgagacctaaagaagtgcttcagataaaaatcatgagtctaagtccaaaattgatactgtaatccaaatgactgaataaagaataaagttgcagcgtgtgtctaaagcagaaagtcaagatgccaacaaagcagaaaagttggagagggccaccagcaagtctagaacttgagcatagatatccatggcttattttagatggttcatgtatgctctgcaaaattcaaagacagcttcgggctttctccgctgataaagttttgttgtaaaagacagacatagaagtcaggctgacagaaaatagagtaacagtaatcataaaaattataatatcagctagcatttattgaaatcttaccatgtgtattctgaggcctttaaatatatgaacttaaacctcacaacatccctgtaatgttggtactatcattatatacattttagttttgctttgttttgttttgtttttgagacggagtctcgctctgtcgcccaggctggagtgcagtggcacgatcttggctcactgcaagctcagcctccaacagtcacagagagattgtgcaactggcccgagtgcacacagcaacagagcctactttttgaccaaaagaggccgaattctcaaccactatactgtagagcctctgtacatgatagaagcaataactgaacctcagtaactctgtggaagtgatttgtaatttgcatgtcgtttcaagcacactctatatctatattttattctattaacttcctaagtctttttaatgaatagcaaaaaaagtatatttcagcattatcaatttaagttaatcataacatctaataggtgacatgtttattttttcatgagttctaattacattttcaagttaagatcactgacttattccctaagtgctcgatctaatgttctgaatcacaggagtatagatgcattattcaccaatattgtttacttttttaaataagattttttttcatggtagaatcatcatatacattcaaaaaggtagacaaaagagagacactcacagtgtcactgttaccaagtgccaggacagatatcagtaaggcaccagcagtcatttttagtgatcaaataccaaacatggtaagataccagagtatttttcacatgacacccaaaaacatattctctgctatcgtggtagagcagaaaaagaacactttcttttcattttacagtatattacgaacctgttccacgtcattaaatattcttctaaaaacccaattttggaataatattctattttattcatatactgtaatttagctaatcaatcttgtatttttgaacatttaggttttttccaatttttatcattataaaaaatgctgtaattaacatccttatttgttcatgtccacatttctttttatgaaaattcagagaaaggaaatgtctggaccaaatggcatgtacaatattaacacttctgttatgtggtttcatattctgcagaaatattactgccacttctacattgtacaagagatctcatagccctaaacatttgctttcatagataattttctgttttttagaaatcttttgtctctttggttatagaaaaagagaacctctttttaattttattttttaaattactagtcaggttgaatgtttcttgttcctaggtcattaacagtttttattttggcaattgtctacagcccatttttctgctgtgttcattttctaatcgaattatgggatttatcttgtaaaaagcctattaagcctttttcatactttacacaagagatcacatctacttactttaccttaaaataacatttacttattttcttataaaagtaatatattatgtttgaaaagaaaaataatatgcaataatatacaataatatgtataatcataatagacaataataaaaatgaaaaaacaccaaccagaggtaattaacattaacatcataatattcttccaggctttttcagatagacaggtaggtaagtaggttggtaggtaaatagatagatagatgatacatagagagagatagatagataatttaaacaaaaataagatcatactgctacattacttcaagccttgcttttatcacttaatatatcttaaacatcttctacatgaacaagtattcatgtgcaacattaattttgatggttgaatagtattatacagatttatcgtaattgacataactattctactgttagacatttggtttatttctatttttccattataaaatgtaatgtttggatcatcctgttgctaaattttgtaagcatttatcttaagtgatttctttagctataactcctagaagtgaaatttctagattaaaggataaatgcacttttaatactttttaaatatattaaaaaattaccttcctgaattattttattaacatgtacctctcctagtcagataagacagcccatttatttcagtctcaccaatactgagtatagcattttaatcattgccaatataatagctaatagcttaaagttatttcatagcttatatttacacttctttaaatactaataaaattgaacattgtttaatgttttgaggatacttgattggtgatttgcctcctactattatttttctactgggtatatctttttcttgttgctttgtaagagcactttatatattaaggagttaaattttgtgatatatgtttcaactacttttatcatgttaaaaatatagacttctatttgtcttttattgtaggttttatcaatcagaaaagaatgttgacttttccgtccattcagatgattgtggcttttttccctaatggatttgttaatatgttcaattgtatgaatttatttcctaatgtagcgatatccttgtcttctaggaataaactttttgggcgaatcatatcaatcttctacttaactaccaaattaatttacatatatttgcatcaatattaagttcataaaattaaaagtttatcattttcatctctgttagagttttgtattggcattagcatttgtattagcattgtattaggaaaaactcaggagtttttctctataatcttggttagtttatttttaaataattgttatttatgaaagtaatacataaaaatgttgtttaaaaaaactcacaagagggtaaaaattaaaaaaataataaaagttcccttcttccttttcatctctagtcccagcctaagtgctaaccacttcacccatttatagtgttagttcttctggtgattaccagtgcaattctaacaaataggtttgcatttctctttctgaattgatcaattttttaaaaatatgcagcactctgactcaaactccctttctttttcctgagatatgttaacttctaatgttatattgtttatcacaactttcacaaatatttctaaataaaggttacctactgattctggattgtgctacataaaattagagcttccaaatttccctccatcacttctccctgctctctactctcacaatcagtcaactatacacctatttctacgctgtcaactatatgttcatttctatgaatctgtaatatttgcattttgttctggaacaaattaagatttaggtgctttgactagattgattctaaacatcaaaaaacaaaaataaaatatctgcaattatgtgatcatgtatttatgcattgccaaataatggtattccattattttaatttgataagataaatttaatatacattaattcttatttcctctattttatggaagaaatatttttaaaaatcacaaaacttttacattcgcttctcttcctgatatttatttttcctttttcttctgcctctcttctttcgtgcccttttttccgaaaattattttgatgattttttttattcaccatacataattacaaatgaagagttgctctggttcgtattggtagtagaagtggggtccctttgctgttttgttggtatattcacccagtgtgacctcttttccgtgggatggctacctctgggctctgattatgtggctcatagggaagtataggcagcctgagtcagaatccccacagttccaaagtgatggaagactgtactctgagatacaatattttgacgtatttcactcccagggagagctacttgctattctttcactgcttccattacccaaaagagtatgatattctccccattttctgtattgcctcacacctaggacccttcaaacccacaccaacgacctctcaggcaaattgatctattcagatagcagtttacagggatagacatgaggcctcttggcccacctgctctatacacaatcttctagtcgaccatcctgattgacaacccacagcccacccctgtctctgtatttgctgactcagagcatggagactttatacatgtgaaatcacatctcccctcccctcacgcttggcttgtgtgtattgcaaaagctgcaatttagtttttcctgtgccttgtcttacccatggataagaacactaagatactttccgctttttgaatatttgtcaaagtgatggcacttggattggtttcagcatgtacatagattgattcttattcatatacatttactgtcatattggggaaatcttgaggggaaaagaaaagcctcttactaccatgttcatcaagagtcctctctggaatagttgaaatagtttaggagttatcggttcactccttcgtcttgtttgcctccagatttcttttttccttcttttcctgtttcttaatgttggaacagcccacacctcaatccttggatgacatctcccttttatctgtattcatccctagtgatctcttctggttgcaaggctataaatgccatctgtatgctgagatttttttatttttagttcaaacctctctccccaactttacactcacatttctaactgtgtgttatagatgatagctttagattctcctcctcaggactaggctttctgcttgatctaggagcaaagctgaaaactgctatggagcttggtgttgccttgataccgaggaggagtctcagagcctctccagtgcaaatacacacttgcagtgtcctagtgctgctgacttacgtactcattctaaagagggtagattgtatccattttaatagctctcaatacccttgcccattcccattacagctggcagaaattccccagctgcaaatgggttgcatttatctgttacccattcctgatttgcagcacgattgactgtttccttttctgcgtggtctatgtgtcactccatgagaatctgggagaggagaactcgatatggtgctcattccatcattttaaccatagtgttctttattgtgaagaggtatcccggttagagtggagtcaaagctttatagcaagtgtatttcaggtaactattcttttacttgtttcagatctatttcagtagcacagccaccaagatagaacacttataagcccttattttggtatagatcaccagagaaaccactcaccactcgcacttggccttcagtactgagtcattacatattggaaggtcagaaactacatcaacaggaaagacccataggtgtgggggtctgatatatttgccagtacatttcttatcattacaaacaaagatttatgtgtgtctagatgt

SEQ ID NO: 78 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gctagggtggtcttttagaaatgca

SEQ ID NO: 79 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggtcaagctagggtggtcttttaga

SEQ ID NO: 80 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ctgcaggaaatggtcaagctagggt

SEQ ID NO: 81 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gaagtggtgagtgacgctcctgcag

SEQ ID NO: 82 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gttaggaagacagaagtggtgagtg

SEQ ID NO: 83 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
atcctggcaaaaactgttaggaaga

SEQ ID NO: 84 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gtgggattcaggagcccgcaggatc

SEQ ID NO: 85 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggtgacattgtgggattcaggagcc

SEQ ID NO: 86 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggagccaaaggtgacattgtgggat

SEQ ID NO: 87 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggtcacaaaggagccaaaggtgaca

SEQ ID NO: 88 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
acagtgcagggtcacaaaggagcca

SEQ ID NO: 89 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ctgttgctgtacagtgcagggtcac

SEQ ID NO: 90 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gggacaggaatgcctgttgctgtac

SEQ ID NO: 91 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
caggtgatggtggggacaggaatgc

SEQ ID NO: 92 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ccgttttcaatccaggtgatggtgg

SEQ ID NO: 93 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
tgacactcacagcatttccgttttc

SEQ ID NO: 94 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
aagtccccacacacatgacactcac

SEQ ID NO: 95 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggtcttccccagacaagtccccaca

SEQ ID NO: 96 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
actatgtcagtagatttgaagggaa

SEQ ID NO: 97 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
tcccactatactatgtcagtagatt

SEQ ID NO: 98 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
tcagtcaaggatttcccactatact

SEQ ID NO: 99 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
aaaagaactcagtcaaggatttccc

SEQ ID NO: 100 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gtaaaggaaaataaaagaactcagt

SEQ ID NO: 101 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
aacctgacagagtaaaggaaaataa

SEQ ID NO: 102 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ggacccagaagaaacctgacagagt

SEQ ID NO: 103 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
cactctcttgaatggacccagaaga

SEQ ID NO: 104 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
cactcggtctttcacactctcttga

SEQ ID NO: 105 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gtcttgagtcaatcactcggtcttt

SEQ ID NO: 106 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gataaacagctgcagtcttgagtca

SEQ ID NO: 107 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
agtcctggcttggtgataaacagct

SEQ ID NO: 108 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
atgtgtagagtcctggcttggtgat

SEQ ID NO: 109 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gtagctatgcatgtgtagagtcctg

SEQ ID NO: 110 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
tgcttattggtagctatgcatgtgt

SEQ ID NO: 111 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
acttctccccatgcttattggtagc

SEQ ID NO: 112 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ccttggcagtactgaacttctcccc

SEQ ID NO: 113 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
cctgctatgctgatggtggctgcag

SEQ ID NO: 114 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gggcatcctacctgctatgctgatg

SEQ ID NO: 115 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gcaaatgtgaaggggcatcctacct

SEQ ID NO: 116 - protein, 91 aa, Homo sapiens
ARILRAPESHNVTFGSFVTLHCTATGIPVPTITWIENGNAVSSGSIQESVKDRVIDSRLQLFITKPGLYTCIATNKHGEKFSTAKAAATIS

SEQ ID NO: 117 - protein, 91 aa, Mus musculus
ARILRAPESHNVTFGSFVTLRCTAIGIPVPTISWIENGNAVSSGSIQESVKDRVIDSRLQLFITKPGLYTCIATNKHGEKFSTAKAAATVS

SEQ ID NO: 118 - DNA, 25 bp, synthetic construct
  source 1..25: mol_type = other DNA
cctgcaagtgaagatgaaacctaaa

SEQ ID NO: 119 - DNA, 21 bp, synthetic construct
  source 1..21: mol_type = other DNA
atgaatcctcaagctcccaga

SEQ ID NO: 120 - DNA, 20 bp, synthetic construct
  source 1..20: mol_type = other DNA
ggctcctgaatcccacaatg

SEQ ID NO: 121 - DNA, 23 bp, synthetic construct
  source 1..23: mol_type = other DNA
gaatggacccagaagaaacagca

SEQ ID NO: 122 - DNA, 20 bp, synthetic construct
  source 1..20: mol_type = other DNA
cctcaacgaccactttgtca

SEQ ID NO: 123 - DNA, 20 bp, synthetic construct
  source 1..20: mol_type = other DNA
ttactccttggaggccatgt

SEQ ID NO: 124 - DNA, 20 bp, synthetic construct
  source 1..20: mol_type = other DNA
cgaagctgaagcaggagaag

SEQ ID NO: 125 - DNA, 20 bp, synthetic construct
  source 1..20: mol_type = other DNA
tttgtgggacagcatggatg

SEQ ID NO: 126 - RNA, 65 nt, synthetic construct
  source 1..65: mol_type = other RNA
actctgtcaggtttcttctgggtccattcaagagagtgtgaaagaccgagtgattgactcaagac

SEQ ID NO: 127 - DNA, 20 bp, Homo sapiens
  source 1..20: mol_type = other DNA
ttgaatggacccagaagaaa

SEQ ID NO: 128 - DNA, 20 bp, Homo sapiens
  source 1..20: mol_type = other DNA
cttgaatggacccagaagaa

SEQ ID NO: 129 - DNA, 20 bp, Homo sapiens
  source 1..20: mol_type = other DNA
tcttgaatggacccagaaga

SEQ ID NO: 130 - DNA, 20 bp, Homo sapiens
  source 1..20: mol_type = other DNA
ctcttgaatggacccagaag

SEQ ID NO: 131 - DNA, 20 bp, Homo sapiens
  source 1..20: mol_type = other DNA
tctcttgaatggacccagaa

SEQ ID NO: 132 - DNA, 20 bp, Homo sapiens
  source 1..20: mol_type = other DNA
ctctcttgaatggacccaga

SEQ ID NO: 133 - DNA, 20 bp, Homo sapiens
  source 1..20: mol_type = other DNA
actctcttgaatggacccag

SEQ ID NO: 134 - DNA, 20 bp, Homo sapiens
  source 1..20: mol_type = other DNA
cactctcttgaatggaccca

SEQ ID NO: 135 - DNA, 20 bp, Homo sapiens
  source 1..20: mol_type = other DNA
acactctcttgaatggaccc

SEQ ID NO: 136 - DNA, 20 bp, Homo sapiens
  source 1..20: mol_type = other DNA
cacactctcttgaatggacc

SEQ ID NO: 137 - DNA, 20 bp, Homo sapiens
  source 1..20: mol_type = other DNA
tcacactctcttgaatggac

SEQ ID NO: 138 - DNA, 20 bp, Homo sapiens
  source 1..20: mol_type = other DNA
ttcacactctcttgaatgga

SEQ ID NO: 139 - DNA, 20 bp, Homo sapiens
  source 1..20: mol_type = other DNA
tttcacactctcttgaatgg

SEQ ID NO: 140 - DNA, 20 bp, Homo sapiens
  source 1..20: mol_type = other DNA
ctttcacactctcttgaatg

SEQ ID NO: 141 - DNA, 20 bp, Homo sapiens
  source 1..20: mol_type = other DNA
tctttcacactctcttgaat

SEQ ID NO: 142 - DNA, 20 bp, Homo sapiens
  source 1..20: mol_type = other DNA
gtctttcacactctcttgaa

SEQ ID NO: 143 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
ttgaatggacccagaagaaa

SEQ ID NO: 144 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
cttgaatggacccagaagaa

SEQ ID NO: 145 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
tcttgaatggacccagaaga

SEQ ID NO: 146 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
ctcttgaatggacccagaag

SEQ ID NO: 147 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
tctcttgaatggacccagaa

SEQ ID NO: 148 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
ctctcttgaatggacccaga

SEQ ID NO: 149 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
actctcttgaatggacccag

SEQ ID NO: 150 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
cactctcttgaatggaccca

SEQ ID NO: 151 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
acactctcttgaatggaccc

SEQ ID NO: 152 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
cacactctcttgaatggacc

SEQ ID NO: 153 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
tcacactctcttgaatggac

SEQ ID NO: 154 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
ttcacactctcttgaatgga

SEQ ID NO: 155 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
tttcacactctcttgaatgg

SEQ ID NO: 156 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
ctttcacactctcttgaatg

SEQ ID NO: 157 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
tctttcacactctcttgaat

SEQ ID NO: 158 - DNA, 20 bp, Homo sapiens
  source 1..20: mol_type = other DNA
  modified_base 1..20: mod_base = OTHER; note = Phosphorothioate linkage
  modified_base 1..20: mod_base = OTHER; note = 2-prime-MOE modified sugar
gtctttcacactctcttgaa

SEQ ID NO: 159 - DNA, 25 bp, Homo sapiens
  source 1..25: mol_type = other DNA
ccagaagaaacctgacagagtaaag

SEQ ID NO: 160 - DNA, 25 bp, Homo sapiens
  source 1..25: mol_type = other DNA
acccagaagaaacctgacagagtaa

SEQ ID NO: 161 - DNA, 25 bp, Homo sapiens
  source 1..25: mol_type = other DNA
atggacccagaagaaacctgacaga

SEQ ID NO: 162 - DNA, 25 bp, Homo sapiens
  source 1..25: mol_type = other DNA
gaatggacccagaagaaacctgaca

SEQ ID NO: 163 - DNA, 25 bp, Homo sapiens
  source 1..25: mol_type = other DNA
cttgaatggacccagaagaaacctg

SEQ ID NO: 164 - DNA, 25 bp, Homo sapiens
  source 1..25: mol_type = other DNA
ctcttgaatggacccagaagaaacc

SEQ ID NO: 165 - DNA, 25 bp, Homo sapiens
  source 1..25: mol_type = other DNA
ctctcttgaatggacccagaagaaa

SEQ ID NO: 166 - DNA, 25 bp, Homo sapiens
  source 1..25: mol_type = other DNA
cacactctcttgaatggacccagaa

SEQ ID NO: 167 - DNA, 25 bp, Homo sapiens
  source 1..25: mol_type = other DNA
ttcacactctcttgaatggacccag

SEQ ID NO: 168 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ccagaagaaacctgacagagtaaag

SEQ ID NO: 169 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
acccagaagaaacctgacagagtaa

SEQ ID NO: 170 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
atggacccagaagaaacctgacaga

SEQ ID NO: 171 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
gaatggacccagaagaaacctgaca

SEQ ID NO: 172 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
cttgaatggacccagaagaaacctg

SEQ ID NO: 173 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ctcttgaatggacccagaagaaacc

SEQ ID NO: 174 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ctctcttgaatggacccagaagaaa

SEQ ID NO: 175 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
cacactctcttgaatggacccagaa

SEQ ID NO: 176 - DNA, 25 bp, Homo sapiens
  source 1..25: mol_type = other DNA
  modified_base 1..25: mod_base = OTHER; note = Phosphorothioate linkage
  modified_base 1..25: mod_base = OTHER; note = 2-prime-MOE modified sugar
ttcacactctcttgaatggacccag

SEQ ID NO: 177 - DNA, 23 bp, Homo sapiens
  source 1..23: mol_type = other DNA
acccagaagaaacctgacagagt

SEQ ID NO: 178 - DNA, 21 bp, Homo sapiens
  source 1..21: mol_type = other DNA
ccagaagaaacctgacagagt

SEQ ID NO: 179 - DNA, 23 bp, Homo sapiens
  source 1..23: mol_type = other DNA
ggacccagaagaaacctgacaga

SEQ ID NO: 180 - DNA, 21 bp, Homo sapiens
  source 1..21: mol_type = other DNA
acccagaagaaacctgacaga

SEQ ID NO: 181 - DNA, 21 bp, Homo sapiens
  source 1..21: mol_type = other DNA
ggacccagaagaaacctgaca

SEQ ID NO: 182 - DNA, 23 bp, Homo sapiens
  source 1..23: mol_type = other DNA
gaatggacccagaagaaacctga

SEQ ID NO: 183 - DNA, 21 bp, Homo sapiens
  source 1..21: mol_type = other DNA
atggacccagaagaaacctga

SEQ ID NO: 184 - DNA, 23 bp, Homo sapiens
  source 1..23: mol_type = other DNA
ttgaatggacccagaagaaacct

SEQ ID NO: 185 - DNA, 21 bp, Homo sapiens
  source 1..21: mol_type = other DNA
gaatggacccagaagaaacct

SEQ ID NO: 186 - DNA, 21 bp, Homo sapiens
  source 1..21: mol_type = other DNA
ttgaatggacccagaagaaac

SEQ ID NO: 187 - DNA, 23 bp, Homo sapiens
  source 1..23: mol_type = other DNA
ctcttgaatggacccagaagaaa

SEQ ID NO: 188 - DNA, 21 bp, Homo sapiens
  source 1..21: mol_type = other DNA
cttgaatggacccagaagaaa

SEQ ID NO: 189 - DNA, 23 bp, Homo sapiens
  source 1..23: mol_type = other DNA
ctctcttgaatggacccagaaga

SEQ ID NO: 190 - DNA, 21 bp, Homo sapiens
  source 1..21: mol_type = other DNA
ctctcttgaatggacccagaa

SEQ ID NO: 191 - DNA, 21 bp, Homo sapiens
  source 1..21: mol_type = other DNA
ctcttgaatggacccagaaga

SEQ ID NO: 192 - DNA, 23 bp, Homo sapiens
  source 1..23: mol_type = other DNA
cactctcttgaatggacccagaa

SEQ ID NO: 193 - DNA, 21 bp, Homo sapiens
  source 1..21: mol_type = other DNA
cactctcttgaatggacccag

SEQ ID NO: 194 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
acccagaagaaacctgacagagt

SEQ ID NO: 195 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
ccagaagaaacctgacagagt

SEQ ID NO: 196 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
ggacccagaagaaacctgacaga

SEQ ID NO: 197 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
acccagaagaaacctgacaga

SEQ ID NO: 198 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
ggacccagaagaaacctgaca

SEQ ID NO: 199 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
gaatggacccagaagaaacctga

SEQ ID NO: 200 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
atggacccagaagaaacctga

SEQ ID NO: 201 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
ttgaatggacccagaagaaacct

SEQ ID NO: 202 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
gaatggacccagaagaaacct

SEQ ID NO: 203 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
ttgaatggacccagaagaaac

SEQ ID NO: 204 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
ctcttgaatggacccagaagaaa

SEQ ID NO: 205 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
cttgaatggacccagaagaaa

SEQ ID NO: 206 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
ctctcttgaatggacccagaaga

SEQ ID NO: 207 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
ctctcttgaatggacccagaa

SEQ ID NO: 208 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
ctcttgaatggacccagaaga

SEQ ID NO: 209 - DNA, 23 bp, Homo sapiens
  source 1..23: mol_type = other DNA
  modified_base 1..23: mod_base = OTHER; note = Phosphorothioate linkage
  modified_base 1..23: mod_base = OTHER; note = 2-prime-MOE modified sugar
cactctcttgaatggacccagaa

SEQ ID NO: 210 - DNA, 21 bp, Homo sapiens
  source 1..21: mol_type = other DNA
  modified_base 1..21: mod_base = OTHER; note = Phosphorothioate linkage
  modified_base 1..21: mod_base = OTHER; note = 2-prime-MOE modified sugar
cactctcttgaatggacccag

SEQ ID NO: 211 - RNA, 71 nt, synthetic construct
  source 1..71: mol_type = other RNA
ggggagaagttcagtactgccaaggctgcagccaccatcagcatagcaggtaggatgccccttcacatttg

SEQ ID NO: 212 - DNA, 20 bp, synthetic construct
  source 1..20: mol_type = other DNA
tttcttctgggtccattcaa

SEQ ID NO: 213 - DNA, 20 bp, synthetic construct
  source 1..20: mol_type = other DNA
ttcttctgggtccattcaag

SEQ ID NO: 214 - DNA, 20 bp, synthetic construct
  source 1..20: mol_type = other DNA
tcttctgggtccattcaaga

SEQ ID NO: 215 - DNA, 20 bp, synthetic construct
  source 1..20: mol_type = other DNA
cttctgggtccattcaagag

SEQ ID NO: 216 - DNA, 20 bp, synthetic construct
  source 1..20: mol_type = other DNA
ttctgggtccattcaagaga

SEQ ID NO: 217 - DNA, 20 bp, synthetic construct
  source 1..20: mol_type = other DNA
tctgggtccattcaagagag

SEQ ID NO: 218 - DNA, 20 bp, synthetic construct
  source 1..20: mol_type = other DNA
ctgggtccattcaagagagt

SEQ ID NO: 219 - DNA, 20 bp, synthetic construct
  source 1..20: mol_type = other DNA
tgggtccattcaagagagtg

SEQ ID NO: 220 - DNA, 20 bp, synthetic construct
  source 1..20: mol_type = other DNA
gggtccattcaagagagtgt

SEQ ID NO: 221 - DNA, 20 bp, synthetic construct
  source 1..20: mol_type = other DNA
ggtccattcaagagagtgtg

SEQ ID NO: 222 - DNA, 20 bp, synthetic construct
  source 1..20: mol_type = other DNA
gtccattcaagagagtgtga

SEQ ID NO: 223 - DNA, 20 bp, synthetic construct
  source 1..20: mol_type = other DNA
tccattcaagagagtgtgaa

SEQ ID NO: 224 - DNA, 20 bp, synthetic construct
  source 1..20: mol_type = other DNA
ccattcaagagagtgtgaaa

SEQ ID NO: 225 - DNA, 20 bp, synthetic construct
  source 1..20: mol_type = other DNA
cattcaagagagtgtgaaag

SEQ ID NO: 226 - DNA, 20 bp, synthetic construct
  source 1..20: mol_type = other DNA
attcaagagagtgtgaaaga

SEQ ID NO: 227 - DNA, 20 bp, synthetic construct
  source 1..20: mol_type = other DNA
ttcaagagagtgtgaaagac

SEQ ID NO: 228 - DNA, 25 bp, synthetic construct
  source 1..25: mol_type = other DNA
ctttactctgtcaggtttcttctgg

SEQ ID NO: 229 - DNA, 25 bp, synthetic construct
  source 1..25: mol_type = other DNA
ttactctgtcaggtttcttctgggt

SEQ ID NO: 230 - DNA, 25 bp, synthetic construct
  source 1..25: mol_type = other DNA
tctgtcaggtttcttctgggtccat

SEQ ID NO: 231 - DNA, 25 bp, synthetic construct
  source 1..25: mol_type = other DNA
tgtcaggtttcttctgggtccattc

SEQ ID NO: 232 - DNA, 25 bp, synthetic construct
  source 1..25: mol_type = other DNA
caggtttcttctgggtccattcaag

SEQ ID NO: 233 - DNA, 25 bp, synthetic construct
  source 1..25: mol_type = other DNA
ggtttcttctgggtccattcaagag

SEQ ID NO: 234 - DNA, 25 bp, synthetic construct
  source 1..25: mol_type = other DNA
tttcttctgggtccattcaagagag

SEQ ID NO: 235 - DNA, 25 bp, synthetic construct
  source 1..25: mol_type = other DNA
ttctgggtccattcaagagagtgtg

SEQ ID NO: 236 - DNA, 25 bp, synthetic construct
  source 1..25: mol_type = other DNA
ctgggtccattcaagagagtgtgaa

SEQ ID NO: 237 - DNA, 23 bp, synthetic construct
  source 1..23: mol_type = other DNA
actctgtcaggtttcttctgggt

SEQ ID NO: 238 - DNA, 21 bp, synthetic construct
  source 1..21: mol_type = other DNA
actctgtcaggtttcttctgg

SEQ ID NO: 239 - DNA, 23 bp, synthetic construct
  source 1..23: mol_type = other DNA
tctgtcaggtttcttctgggtcc

SEQ ID NO: 240 - DNA, 21 bp, synthetic construct
  source 1..21: mol_type = other DNA
tctgtcaggtttcttctgggt

SEQ ID NO: 241 - DNA, 21 bp, synthetic construct
  source 1..21: mol_type = other DNA
tgtcaggtttcttctgggtcc

SEQ ID NO: 242 - DNA, 23 bp, synthetic construct
  source 1..23: mol_type = other DNA
tcaggtttcttctgggtccattc

SEQ ID NO: 243 - DNA, 21 bp, synthetic construct
  source 1..21: mol_type = other DNA
tcaggtttcttctgggtccat

SEQ ID NO: 244 - DNA, 23 bp, synthetic construct
  source 1..23: mol_type = other DNA
aggtttcttctgggtccattcaa

SEQ ID NO: 245 - DNA, 21 bp, synthetic construct
  source 1..21: mol_type = other DNA
aggtttcttctgggtccattc

SEQ ID NO: 246 - DNA, 21 bp, synthetic construct
  source 1..21: mol_type = other DNA
gtttcttctgggtccattcaa

SEQ ID NO: 247 - DNA, 23 bp, synthetic construct
  source 1..23: mol_type = other DNA
tttcttctgggtccattcaagag

SEQ ID NO: 248 - DNA, 21 bp, synthetic construct
  source 1..21: mol_type = other DNA
tttcttctgggtccattcaag

SEQ ID NO: 249 - DNA, 23 bp, synthetic construct
  source 1..23: mol_type = other DNA
tcttctgggtccattcaagagag

SEQ ID NO: 250 - DNA, 21 bp, synthetic construct
  source 1..21: mol_type = other DNA
ttctgggtccattcaagagag

SEQ ID NO: 251 - DNA, 21 bp, synthetic construct
  source 1..21: mol_type = other DNA
tcttctgggtccattcaagag

SEQ ID NO: 252 - DNA, 23 bp, synthetic construct
  source 1..23: mol_type = other DNA
ttctgggtccattcaagagagtg

SEQ ID NO: 253 - DNA, 21 bp, synthetic construct
  source 1..21: mol_type = other DNA
ctgggtccattcaagagagtg

SEQ ID NO: 254 - RNA, 256 nt, Homo sapiens
  source 1..256: mol_type = other RNA
atggtctaatttgcatttctaaaagaccaccctagcttgaccatttcctgcaggagcgtcactcaccacttctgtcttcctaacagtttttgccaggatcctgcgggctcctgaatcccacaatgtcacctttggctcctttgtgaccctgcactgtacagcaacaggcattcctgtccccaccatcacctggattgaaaacggaaatgctgtgagtgtcatgtgtgtggggacttgtctggggaagacccattgg

SEQ ID NO: 255 - RNA, 272 nt, Homo sapiens
  source 1..272: mol_type = other RNA
atgcttttcccttcaaatctactgacatagtatagtgggaaatccttgactgagttcttttattttcctttactctgtcaggtttcttctgggtccattcaagagagtgtgaaagaccgagtgattgactcaagactgcagctgtttatcaccaagccaggactctacacatgcatagctaccaataagcatggggagaagttcagtactgccaaggctgcagccaccatcagcatagcaggtaggatgccccttcacatttgcgttgttcc

SEQ ID NO: 256 - RNA, 96 nt, Homo sapiens
  source 1..96: mol_type = other RNA
gactgagttcttttattttcctttactctgtcaggtttcttctgggtccattcaagagagtgtgaaagaccgagtgattgactcaagactgcagct

SEQ ID NO: 257 - RNA, 176 nt, Homo sapiens
  source 1..176: mol_type = other RNA
gactgagttcttttattttcctttactctgtcaggtttcttctgggtccattcaagagagtgtgaaagaccgagtgattgactcaagactgcagctggggagaagttcagtactgccaaggctgcagccaccatcagcatagcaggtaggatgccccttcacatttgcgttgttcc

SEQ ID NO: 258 - DNA, 55 bp, synthetic construct
  source 1..55: mol_type = other DNA
agtggtgaagctggaagttgaggaatggagtaaaccacagaaagataacaaaggc

(19) United States
(12) Patent Application Publication
(10) Pub. No.: US 2025/0257359 A1
(43) Pub. Date: Aug. 14, 2025

(54) MUSK-TARGETING OLIGONUCLEOTIDES

(71) Applicant: Bolden Therapeutics, Inc., Providence, RI (US)

(72) Inventors: John Page, Providence, RI (US); Anne Valat, Cambridge, MA (US); Sudhir Agrawal, Shrewsbury, MA (US); Duncan Brown, Berkeley, CA (US)

(21) Appl. No.: 18/730,084

(22) PCT Filed: Jan. 20, 2023
(86) PCT No.: PCT/US23/11286

§ 371 Date: Jul. 18, 2024

Related U.S. Application Data
(60) Provisional application No. 63/301,416, filed on Jan. 20, 2022.

(51) Int. Cl. C12N 15/113

(52) U.S. Cl. CPC C12N 15/1138; C12Y 207/10001; C12N 2310/14; C12N 2310/313; C12N 2310/321; C12N 2320/33

(57) ABSTRACT

The present disclosure provides, among other things, MuSK-targeting oligonucleotide compositions that alter the splicing of MuSK transcripts through exon skipping, and methods of treating diseases with said compositions.

### CROSS REFERENCE TO RELATED APPLICATIONS

This application is a National Phase entry of International Application No. PCT/US23/11286, filed Jan. 20, 2023, which claims the benefit of U.S. Provisional Application No. 63/301,416, filed Jan. 20, 2022, the contents of which are hereby incorporated by reference in their entirety.

### BACKGROUND

Neurodegenerative diseases represent a major public health challenge, expected to impact one in five people in their lifetimes. Alzheimer's disease (AD) is the most common cause of age-related dementia. There is a critical and urgent need for therapeutics to prevent and treat AD and other neurodegenerative conditions.

Skeletal muscle has the ability to regenerate after injury. Muscle regeneration is dependent upon resident stem cells, referred to as muscle satellite cells. In mature muscle tissue, satellite cells constitute a small, scattered population of mitotically and physiologically quiescent cells. Satellite cells are also implicated in normal muscle growth and maintenance throughout life, indicating that they could be exploited to treat muscle wasting conditions.

Skeletal muscle makes up about 35% of body weight and is essential for metabolism, locomotion, and breathing, which highlights its importance in human health. Muscle wasting reduces mobility, metabolism, and quality of life for the majority of cancer patients, elderly patients, and many others with no history of neuromuscular dysfunction. In addition, muscular dystrophies are an often fatal group of genetic diseases leading to severe muscle loss, including Duchenne Muscular Dystrophy which affects children.

Neurogenesis and muscle regeneration are dependent on neural stem cells (NSCs) and muscle satellite cells. A roadblock to development of treatments of neurodegeneration and muscle wasting is that the signaling that regulates neural stem cells and satellite cells and their regeneration is poorly understood. Accordingly, there is a need for compositions and methods for promoting neurogenesis and muscle regeneration.

### SUMMARY

Among other things, the present disclosure provides an insight that presence and/or activity of a particular form(s) of the muscle-specific tyrosine kinase (MuSK) protein, specifically lacking a functional Ig3 domain, may achieve or contribute to beneficial biological events including, for example, neurogenesis and/or muscle regeneration.

The present disclosure provides certain technologies for enhancing neurogenesis, including in particular in adult humans. In some embodiments, technologies provided herein may be useful in medicine, including specifically treatment of diseases, disorders or conditions associated with neurodegeneration, or otherwise with low or reduced neuronal activity (e.g., neuronal activity in an adult hippocampus and/or in subventricular zone(s)). For example, in some embodiments, technologies provided herein may be useful in the treatment of one or more of Alzheimer's Disease (AD), Parkinson's disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy, Hippocampal Sclerosis, Niemann Pick Type C, Diabetes-mediated hippocampal neuronal loss, brain injury (e.g., traumatic and/or anoxic brain injury), and Huntington's disease.

Bone morphogenetic protein (BMP) signaling regulates at least two important NSC decision points: 1) quiescence, where proliferating stem cells exit the cell cycle and return to replenish a reserve pool that can supply fresh stem cells; and: 2) differentiation into mature progeny (Mira et al., 2010). The present disclosure contemplates that manipulating the BMP pathway in NSCs is an attractive target for regulating neurogenesis in the adult brain. Additionally, BMP signaling regulates skeletal muscle stem cell activity in both normal and pathological states. The present disclosure provides technologies for increasing level and/or activity of MuSK form(s) that functionally participate in neurogenesis and/or muscle regeneration, including, in some embodiments, by reducing alternative splicing that would otherwise generate MuSK form(s) that do not so participate. In some embodiments, such increase is in a relevant tissue such as muscle. Alternatively or additionally, in some embodiments, such increase is in a tissues such as a brain tissue (e.g., hippocampal and/or subventricular) and/or lung tissue.

Among other things, the present disclosure provides an insight that presence and/or activity of a particular form(s) of the muscle-specific tyrosine kinase (MuSK) protein, specifically lacking a functional Ig3 domain, may achieve or contribute to neurogenesis in adult humans, or otherwise provide neurological benefit(s). The MuSK transcript can be alternatively spliced, including to generate at least one form (i.e., ΔIg3-MuSK) that lacks the Ig3 domain. The present disclosure appreciates that increasing presence and/or level of ΔIg3-MuSK, and/or of other functional form(s) in which its Ig3 domain is altered (e.g., mutated, blocked, etc.) or removed, may provide benefits as described herein.

In some embodiments, the present disclosure provides technologies for increasing level and/or activity of one or more forms of MuSK whose Ig3 domain is altered (e.g., mutated, blocked, removed, etc.) for example so that it fails to effectively participate in interaction(s) with BMP. In some embodiments, the present disclosure provides technologies for reducing Ig3⁺ MuSK, for example by reducing level and/or activity of one or more forms of MuSK whose Ig3 domain effectively participates in interaction(s) with BMP.

In some embodiments, an agent that targets the MuSK Ig3 domain, as described herein (e.g., a MuSK-targeting oligonucleotide), so that level and/or activity of a MuSK/BMP complex is reduced is useful in contexts of neurogenesis and/or muscle regeneration and/or of muscle growth.

In some embodiments, provided agent(s) may enhance muscle growth. In some embodiments, muscle growth occurs in uninjured tissue. In some embodiments, muscle growth occurs in injured tissue. In some embodiments, enhanced and/or increased muscle growth is determined by a decrease in satellite cell number and/or increase in muscle fiber size. In this regard, muscle growth can be characterized by a decrease in satellite cell number and/or increase in muscle fiber size, which is indicative of satellite cells differentiating and fusing into/augmenting existing muscle fibers and forming new muscle fibers.

Embodiments of the present invention provide methods of enhancing neurogenesis and/or muscle regeneration and/or growth, for example in a subject in need thereof, by administering a composition that downregulates MuSK Ig3 domain protein expression, MuSK Ig3 domain gene expression, and/or MuSK Ig3 activation of BMP signaling, thereby upregulating muscle satellite cells which results in enhancement of muscle regeneration and/or growth. In some embodiments, such a composition can comprise and/or deliver a MuSK-targeting oligonucleotide (e.g., a MuSK Ig3-targeting exon-skipping oligonucleotide).

In some embodiments, enhancing neurogenesis is used in the context of treating a disease or disorder associated with reduced Adult Hippocampal Neurogenesis (AHN). As AHN occurs throughout life in humans and is dramatically reduced in AD (Moreno-Jimenez et al., 2019: Steiner et al., 2019), the present disclosure provides compositions and methods for promoting AHN, enhancing cognitive function and combating neurodegeneration (e.g., Alzheimer's disease).

In some embodiments, enhancing neurogenesis is used in the context of treating a disease or disorder associated with reduced Subventricular Zone Neurogenesis. As NSCs reside in the subventricular zone (SVZ) lining the lateral ventricles and generate astrocytes and oligodendrocytes that support the existing circuitry as well as neurons in the olfactory bulb that are critical for olfactory discrimination. The present disclosure provides compositions and methods for compensating for the degeneration of neurons in the SVZ through enhancing endogenous neurogenesis. In some embodiments, the present disclosure provides compositions and methods for treating diseases specifically associated with striatal neurogenesis such as Parkinson's disease (which could benefit both from increasing AHN and striatal neurogenesis in the SVZ: Pitcher et al. 2012: Sterling et al. 2013) and Huntington's disease (Sassone et al., 2018). In some embodiments, the present disclosure provides compositions and methods for treating other diseases including addiction (e.g., chronic cocaine use and lifelong cigarette smoking).

In some embodiments, a subject of interest can be at risk of, or afflicted with, a disease or disorder including, but not limited to, neuromuscular dysfunction, neurodegenerative disorder, cardiac disorder (e.g., myocardial infarction, cardiomyopathy), or genetic diseases characterized by muscle wasting. Alternatively or additionally, in some embodiments, a subject of interest can be at risk of, or afflicted with a disease or disorder associated with lung damage, including, for example, idiopathic pulmonary fibrosis (IPF), acute respiratory distress syndrome (ARDS), pneumonia, and/or certain infections, including viral infections including coronaviral infections such as COVID19.

Exemplary neuromuscular dysfunctions or disorders that can be treated by technologies of the present invention include, but are not limited to, Becker muscular dystrophy, Congenital muscular dystrophy, Distal muscular dystrophy, Duchenne muscular dystrophy, Emery-Dreifuss muscular dystrophy, Facioscapulohumeral muscular dystrophy, Limb-girdle muscular dystrophy, Myotonic muscular dystrophy, and Oculo-pharyngeal muscular dystrophy.

In some embodiments, enhancing muscle growth is used in the context of treating a disease or disorder associated with muscle atrophy or muscle wasting. Muscle atrophy or muscle wasting may be observed in connection with various diseases and conditions described herein, such as neuromuscular disorders, or direct or indirectly caused by prolonged inactivity, bed rest, hospitalization, aging, malnutrition, cancer cachexia, chronic inflammatory diseases, etc. Example chronic inflammatory diseases include rheumatoid arthritis, chronic heart failure, and chronic obstructive pulmonary disease (COPD).

Duration of hospitalization and type and severity of the illness can affect the extent of muscle wasting in a subject, and muscle wasting is common in patients suffering from sepsis, organ failure, hyperglycemia, and diseases associated with chronic and systemic inflammation or oxidative stress. Additionally, hospitalization requiring complete immobilization/bed rest contributes significantly to muscle wasting.

Additional disorders associated with muscle atrophy/wasting include disorders associate with decreased mobility, such as rheumatoid arthritis, osteoarthritis, and injury. (2016 Powers, Scott K., et al. Medicine and science in sports and exercise 48(11): 2307). Thus, in some embodiments, the present disclosure provides therapies for preventing/treating muscle wasting or muscle atrophy related to or as a result of a number of diseases or conditions described herein.

In some embodiments, methods of the present invention can also be used when a subject is in need of enhanced muscle regeneration and muscle growth following surgery, trauma and/or prolonged immobilization (e.g., from bed-rest or casting). As muscle stem cell activity is known to decrease with age, methods of the present invention can also be used to prevent or reverse sarcopenia in patients that are otherwise healthy and could lead to significant improvements in quality of life and autonomy.

Embodiments of the present invention also provide methods of preventing or treating neurodegenerative diseases (e.g., AD) and/or muscle fibrosis, e.g., in a subject in need thereof, by administering a composition that downregulates the MuSK Ig3 domain protein expression, the MuSK Ig3 domain gene expression, and/or the MuSK Ig3 activation of BMP signaling. The composition can comprise, e.g., a MuSK-targeting oligonucleotide (e.g., a MuSK Ig3-targeting exon-skipping oligonucleotide). The subject can be at risk of, or afflicted with, various neurodegenerative diseases, such as Alzheimer's Disease (AD), Parkinson's disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy; Hippocampal Sclerosis, Niemann Pick Type C, Diabetes-mediated hippocampal neuronal loss, brain injury (e.g., traumatic and/or anoxic brain injury), and Huntington's disease or muscle fibrosis resulting from a disease or condition including, but not limited to, trauma, heritable disease, muscle disorder and aging. The trauma can result from, for example, radiation treatment, crush injury, laceration, and amputation. The heritable disease or muscle disorder include, but are not limited to, Congenital Muscular Dystrophy, Duchenne Muscular Dystrophy, Becker's Muscular Dystrophy: Amyotrophic Lateral Sclerosis (ALS), and age-associate sarcopenia.

The present invention features, inter alia, an oligonucleotide composition comprising plurality of oligonucleotides, the oligonucleotide composition being characterized in that, when it is contacted with a MuSK transcript in a transcript splicing system, relative amounts of transcripts that do and do not include Ig3 domain-encoding sequences are altered as compared with such relative amounts observed under reference conditions selected from the group consisting of absence of the composition, presence of a reference composition, and combinations thereof.

In some embodiments, the oligonucleotides mediate skipping of at least one exon of the MuSK gene. In some embodiments, the exon skipping lowers levels of mRNAs encoding MuSK protein form that participate in BMP signaling compared with levels observed absent the exon skipping. In some embodiments, the MuSK protein form participating in BMP signaling is or comprises a MuSK protein form that forms a MuSK/BMP complex.

In some embodiments, the exon skipping reduces the level and/or activity of a MuSK/BMP complex. In some embodiments, at least one skipped exon is selected from the group consisting of exons 3, 4, 6, and 7. In some embodiments, the relative amounts are amounts of transcripts including exons 6 and 7 relative to those lacking exons 6 and 7. In some embodiments, relative amounts are amounts of transcripts including exons 3 and 4 relative to those lacking exons 6 and 7.

In some embodiments, the alteration comprises skipping one or more of exons 6 and 7 of MuSK. In some embodiments, the alteration comprises skipping one or more of exons 3 and 4 of MuSK. In some embodiments, the alteration comprises skipping one or more of exons 6 and 7 of MuSK, but skipping none of exons 3 and 4 of MuSK.

In some embodiments, MuSK splicing is altered in that level of MuSK transcripts including exons 6 and 7 is decreased or level of MuSK protein forms including sequences encoded by exons 6 and 7 is decreased, or both. In some embodiments, MuSK splicing is altered in that level of MuSK transcripts including exons 3 and 4 is decreased or level of MuSK protein forms including sequences encoded by exons 3 and 4 is decreased, or both. In some embodiments, MuSK splicing is altered in that level of MuSK transcripts including exons 6 and 7 is increased or level of MuSK protein forms including sequences encoded by exons 6 and 7 is increased, or both. In some embodiments, MuSK splicing is altered in that level of MuSK transcripts including exons 3 and 4 is increased or level of MuSK protein forms including sequences encoded by exons 3 and 4 is increased, or both. In some embodiments, MuSK splicing is altered in that level of MuSK transcripts including exons 3 and 4 remains substantially unchanged and level of MuSK transcripts including exons 6 and 7 is decreased. In some embodiments, MuSK splicing is altered in that level of MuSK protein forms including sequences encoded by exons 3 and 4 remains substantially unchanged and I level of MuSK protein forms including sequences encoded by exons 6 and 7 is decreased. In some embodiments, MuSK splicing is altered in that total level of MuSK transcripts remained substantially unchanged and level of MuSK transcripts including exons 6 and 7 is decreased.

In some embodiments, MuSK splicing is altered in that ratio of MuSK transcripts including exons 3 and 4 to MuSK transcripts including exons 6 and 7 is increased. In some embodiments, MuSK splicing is altered in that ratio of MuSK transcripts including exons 3 and 4 to MuSK transcripts including exons 6 and 7 is increased by about 10%. In some embodiments, MuSK splicing is altered in that ratio of MuSK transcripts including exons 3 and 4 to MuSK transcripts including exons 6 and 7 is increased by about 50%. In some embodiments, MuSK splicing is altered in that ratio of MuSK transcripts including exons 3 and 4 to MuSK transcripts including exons 6 and 7 is increased by about 80%. In some embodiments, MuSK splicing is altered in that ratio of MuSK transcripts including exons 3 and 4 to MuSK transcripts including exons 6 and 7 is increased by about 100%. In some embodiments, MuSK splicing is altered in that ratio of MuSK transcripts including exons 3 and 4 to MuSK transcripts including exons 6 and 7 is increased by about 120%. In some embodiments, MuSK splicing is altered in that ratio of MuSK transcripts including exons 3 and 4 to MuSK transcripts including exons 6 and 7 is increased by about 150%. In some embodiments, MuSK splicing is altered in that ratio of MuSK transcripts including exons 3 and 4 to MuSK transcripts including exons 6 and 7 is increased by about 180%. In some embodiments, MuSK splicing is altered in that ratio of MuSK transcripts including exons 3 and 4 to MuSK transcripts including exons 6 and 7 is increased by about 200%. In some embodiments, MuSK splicing is altered in that ratio of MuSK transcripts including exons 3 and 4 to MuSK transcripts including exons 6 and 7 is increased by about 250) %. In some embodiments, MuSK splicing is altered in that ratio of MuSK transcripts including exons 3 and 4 to MuSK transcripts including exons 6 and 7 is increased by about 300%. In some embodiments, MuSK splicing is altered in that ratio of MuSK transcripts including exons 3 and 4 to MuSK transcripts including exons 6 and 7 is increased by about 350%. In some embodiments, MuSK splicing is altered in that ratio of MuSK transcripts including exons 3 and 4 to MuSK transcripts including exons 6 and 7 is increased by about 400%. In some embodiments, MuSK splicing is altered in that ratio of MuSK transcripts including exons 3 and 4 to MuSK transcripts including exons 6 and 7 is increased by about 450%. In some embodiments, MuSK splicing is altered in that ratio of MuSK transcripts including exons 3 and 4 to MuSK transcripts including exons 6 and 7 is increased by about 500%. In some embodiments, MuSK splicing is altered in that ratio of MuSK transcripts including exons 3 and 4 to MuSK transcripts including exons 6 and 7 is increased by about 600%. In some embodiments, MuSK splicing is altered in that ratio of MuSK transcripts including exons 3 and 4 to MuSK transcripts including exons 6 and 7 is increased by about 700%. In some embodiments, MuSK splicing is altered in that ratio of MuSK transcripts including exons 3 and 4 to MuSK transcripts including exons 6 and 7 is increased by about 800%. In some embodiments, MuSK splicing is altered in that ratio of MuSK transcripts including exons 3 and 4 to MuSK transcripts including exons 6 and 7 is increased by about 900%. In some embodiments, MuSK splicing is altered in that ratio of MuSK transcripts including exons 3 and 4 to MuSK transcripts including exons 6 and 7 is increased by about 1000%. In some embodiments, MuSK splicing is altered in that ratio of MuSK transcripts including exons 3 and 4 to MuSK transcripts including exons 6 and 7 is increased by at least 1000%.

In some embodiments, MuSK splicing is altered in that ratio of MuSK transcripts including exons 3 and 4 to MuSK transcripts including exons 6 and 7 is decreased. In some embodiments, MuSK splicing is altered in that ratio of MuSK transcripts including exons 3 and 4 to MuSK transcripts including exons 6 and 7 is decreased by about 10%. In some embodiments, MuSK splicing is altered in that ratio of MuSK transcripts including exons 3 and 4 to MuSK transcripts including exons 6 and 7 is decreased by about 50%. In some embodiments, MuSK splicing is altered in that ratio of MuSK transcripts including exons 3 and 4 to MuSK transcripts including exons 6 and 7 is decreased by about 80%. In some embodiments, MuSK splicing is altered in that ratio of MuSK transcripts including exons 3 and 4 to MuSK transcripts including exons 6 and 7 is decreased by about 100%. In some embodiments, MuSK splicing is altered in that ratio of MuSK transcripts including exons 3 and 4 to MuSK transcripts including exons 6 and 7 is decreased by about 120%. In some embodiments, MuSK splicing is altered in that ratio of MuSK transcripts including exons 3 and 4 to MuSK transcripts including exons 6 and 7 is decreased by about 150%. In some embodiments, MuSK splicing is altered in that ratio of MuSK transcripts including exons 3 and 4 to MuSK transcripts including exons 6 and 7 is decreased by about 180%. In some embodiments, MuSK splicing is altered in that ratio of MuSK transcripts including exons 3 and 4 to MuSK transcripts including exons 6 and 7 is decreased by about 200%. In some embodiments, MuSK splicing is altered in that ratio of MuSK transcripts including exons 3 and 4 to MuSK transcripts including exons 6 and 7 is decreased by about 250%. In some embodiments, MuSK splicing is altered in that ratio of MuSK transcripts including exons 3 and 4 to MuSK transcripts including exons 6 and 7 is decreased by about 300%. In some embodiments, MuSK splicing is altered in that ratio of MuSK transcripts including exons 3 and 4 to MuSK transcripts including exons 6 and 7 is decreased by about 350%. In some embodiments, MuSK splicing is altered in that ratio of MuSK transcripts including exons 3 and 4 to MuSK transcripts including exons 6 and 7 is decreased by about 400%. In some embodiments, MuSK splicing is altered in that ratio of MuSK transcripts including exons 3 and 4 to MuSK transcripts including exons 6 and 7 is decreased by about 450%. In some embodiments, MuSK splicing is altered in that ratio of MuSK transcripts including exons 3 and 4 to MuSK transcripts including exons 6 and 7 is decreased by about 500%. In some embodiments, MuSK splicing is altered in that ratio of MuSK transcripts including exons 3 and 4 to MuSK transcripts including exons 6 and 7 is decreased by about 600%. In some embodiments, MuSK splicing is altered in that ratio of MuSK transcripts including exons 3 and 4 to MuSK transcripts including exons 6 and 7 is decreased by about 700%. In some embodiments, MuSK splicing is altered in that ratio of MuSK transcripts including exons 3 and 4 to MuSK transcripts including exons 6 and 7 is decreased by about 800%. In some embodiments, MuSK splicing is altered in that ratio of MuSK transcripts including exons 3 and 4 to MuSK transcripts including exons 6 and 7 is decreased by about 900%. In some embodiments, MuSK splicing is altered in that ratio of MuSK transcripts including exons 3 and 4 to MuSK transcripts including exons 6 and 7 is decreased by about 1000%. In some embodiments, MuSK splicing is altered in that ratio of MuSK transcripts including exons 3 and 4 to MuSK transcripts including exons 6 and 7 is decreased by at least 1000%.

In some embodiments, prior to contact with one or more exon-skipping oligonucleotides as described herein, ratio of MuSK transcripts including exons 3 and 4 (i.e., total MuSK transcripts) to MuSK transcripts in a cell or system including exons 6 and 7 is between about 1:1 and 1:0.1. In some embodiments, prior to contact with one or more exon-skipping oligonucleotides as described herein, ratio of MuSK transcripts including exons 3 and 4 (i.e., total MuSK transcripts) to MuSK transcripts in a cell or system including exons 6 and 7 is about 1:1, 1:0.9, 1:0.8, 1:0.7, 1:0.6, 1:0.5, 1:0.4, 1:0.3, 1:0.2, or 1:0.1. In some embodiments, prior to contact with one or more exon-skipping oligonucleotides as described herein, ratio of MuSK transcripts including exons 3 and 4 (i.e., total MuSK transcripts) to MuSK transcripts in a cell or system including exons 6 and 7 is about 1:0.8.

In some embodiments, after contact with one or more exon-skipping oligonucleotides as described herein, ratio of MuSK transcripts including exons 3 and 4 (i.e., total MuSK transcripts) to MuSK transcripts in a cell or system including exons 6 and 7 is between about 1:0.9 and 1:0.0001. In some embodiments, after contact with one or more exon-skipping oligonucleotides as described herein, ratio of MuSK transcripts including exons 3 and 4 (i.e., total MuSK transcripts) to MuSK transcripts in a cell or system including exons 6 and 7 is about 1:0.9, 1:0.8, 1:0.7, 1:0.6, 1:0.5, 1:0.4, 1:0.3, 1:0.2, or 1:0.1, 1:0.05, 1:0.01, 1:0.001, or 1:0001. In some embodiments, after contact with one or more exon-skipping oligonucleotides as described herein, ratio of MuSK transcripts including exons 3 and 4 (i.e., total MuSK transcripts) to MuSK transcripts in a cell or system including exons 6 and 7 is between about 1:0.9 and 1:0.8, 1:08 and 1:0.7, 1:0.7 and 1:0.6, 1:0.6 and 1:0.5, 1:0.5 and 1:0.4, 1:0.4 and 1:0.3, 1:0.3 and 1:0.2, 1:0.2 and 1:0.1, 1:0.1 and 1:0.05, 1:0.05 and 1:0.01, 1:0.01 and 1:0.001, or 1:0.001 and 1:0001. In some embodiments, after contact with one or more exon-skipping oligonucleotides as described herein, ratio of MuSK transcripts including exons 3 and 4 (i.e., total MuSK transcripts) to MuSK transcripts in a cell or system including exons 6 and 7 is between about 1:0.9 and 1:0.5.

In some embodiments, the level of MuSK transcripts including exons 6 and 7 or the level of MuSK protein forms including sequences encoded by exons 6 and 7, or both, decreases at a level at least 2 fold greater than the decrease observed for the level of MuSK transcripts including exons 3 and 4 or the level of MuSK protein forms including sequences encoded by exons 3 and 4, or both.

In some embodiments, the level of MuSK transcripts including exons 6 and 7 or the level of MuSK protein forms including sequences encoded by exons 6 and 7, or both, decreases at a level at least 3 fold greater than the decrease observed for the level of MuSK transcripts including exons 3 and 4 or the level of MuSK protein forms including sequences encoded by exons 3 and 4, or both. In some embodiments, the level of MuSK transcripts including exons 6 and 7 or the level of MuSK protein forms including sequences encoded by exons 6 and 7, or both, decreases at a level at least 4 fold greater than the decrease observed for the level of MuSK transcripts including exons 3 and 4 or the level of MuSK protein forms including sequences encoded by exons 3 and 4, or both.

In some embodiments, the level of MuSK transcripts including exons 6 and 7 or the level of MuSK protein forms including sequences encoded by exons 6 and 7, or both, decreases at a level at least 5 fold greater than the decrease observed for the level of MuSK transcripts including exons 3 and 4 or the level of MuSK protein forms including sequences encoded by exons 3 and 4, or both. In some embodiments, the level of MuSK transcripts including exons 6 and 7 or the level of MuSK protein forms including sequences encoded by exons 6 and 7, or both, decreases at a level at least 10 fold greater than the decrease observed for the level of MuSK transcripts including exons 3 and 4 or the level of MuSK protein forms including sequences encoded by exons 3 and 4, or both.

In some embodiments, the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 40%.

In some embodiments, the level of MuSK transcripts including exons 6 and 7 or the level of MuSK protein forms including sequences encoded by exons 6 and 7, or both, decreases by greater than 70% and the level of MuSK transcripts including exons 3 and 4 or the level of MuSK protein forms including sequences encoded by exons 3 and 4, or both, decreases by less than 40%.

In some embodiments, the level of MuSK transcripts including exons 6 and 7 or the level of MuSK protein forms including sequences encoded by exons 6 and 7, or both, decreases by greater than 80% and the level of MuSK transcripts including exons 3 and 4 or the level of MuSK protein forms including sequences encoded by exons 3 and 4, or both, decreases by less than 40%.

In some embodiments, the level of MuSK transcripts including exons 6 and 7 or the level of MuSK protein forms including sequences encoded by exons 6 and 7, or both, decreases by greater than 90% and the level of MuSK transcripts including exons 3 and 4 or the level of MuSK protein forms including sequences encoded by exons 3 and 4, or both, decreases by less than 40%.

In some embodiments, the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 30%.

In some embodiments, the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 20%. In some embodiments, the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 10%. In some embodiments, the base sequence of the oligonucleotide comprises a sequence having no more than 5 mismatches from a 18-25 base long portion of the MuSK gene or its complement.

In some embodiments, the oligonucleotides correspond to positions 83776-83800 and on 83854-83878 of the MuSK gene sequence represented in SEQ ID NO: 77.

In some embodiments, the oligonucleotides described herein target a region on the MuSK genomic sequence within a region defined by nucleotides 83841-83905 and 83962-84032 on the MuSK gene sequence represented in SEQ ID NO: 77.

In some embodiments, oligonucleotides target a region on the MuSK genomic sequence within or comprising at least a portion of sequence ACTCTGTCAGGTTTCTTCTGGGTCCATTCAAGAGAGTGTGAAAGACCGAGTGATT GACTCAAGAC (region 1, SEQ ID: 126). In some embodiments, oligonucleotides target a portion of MuSK transcript comprising a sequence that is at least 10%, 15%, 20%, 25%, 30%, 35%, 40%, 45%, or 50% identical to region 1, SEQ ID: 126. In some embodiments, oligonucleotides target a portion of MuSK transcript comprising a sequence that is identical to at least 10, 15, 18, 19, 20, 21, 22, 23, 24, 25, 26, 27, 28, 29, or 30 consecutive bases of region 1, SEQ ID: 126. In some embodiments, oligonucleotides target a portion of MuSK transcript comprising a sequence that is identical to no more than 30 consecutive bases of region 1, SEQ ID: 126. In some embodiments, oligonucleotides target a portion of MUSK transcript comprising a sequence that is identical to at least 15 consecutive bases of region 1, SEQ ID: 126. In some embodiments, oligonucleotides target a portion of MuSK transcript comprising a sequence that is identical to 15-30 consecutive bases of region 1, SEQ ID: 126. In some embodiments, oligonucleotides target a portion of MUSK transcript comprising a sequence that is identical to 15, 16, 17, 18, 19, 20, 21, 22, 23, 24, 25, 26, 27, 28, 29, or 30 consecutive bases of region 1, SEQ ID: 126. In some embodiments, oligonucleotides target a portion of MuSK transcript comprising a sequence that is identical to 15 consecutive bases of region 1, SEQ ID: 126. In some embodiments, oligonucleotides target a portion of MuSK transcript comprising a sequence that is identical to 16 consecutive bases of region 1, SEQ ID: 126. In some embodiments, oligonucleotides target a portion of MuSK transcript comprising a sequence that is identical to 17 consecutive bases of region 1, SEQ ID: 126. In some embodiments, oligonucleotides target a portion of MuSK transcript comprising a sequence that is identical to 18 consecutive bases of region 1, SEQ ID: 126. In some embodiments, oligonucleotides target a portion of MuSK transcript comprising a sequence that is identical to 19 consecutive bases of region 1, SEQ ID: 126. In some embodiments, oligonucleotides target a portion of MuSK transcript comprising a sequence that is identical to 20 consecutive bases of region 1, SEQ ID: 126. In some embodiments, oligonucleotides target a portion of MuSK transcript comprising a sequence that is identical to 21 consecutive bases of region 1, SEQ ID: 126. In some embodiments, oligonucleotides target a portion of MuSK transcript comprising a sequence that is identical to 22 consecutive bases of region 1, SEQ ID: 126. In some embodiments, oligonucleotides target a portion of MuSK transcript comprising a sequence that is identical to 23 consecutive bases of region 1, SEQ ID: 126. In some embodiments, oligonucleotides target a portion of MuSK transcript comprising a sequence that is identical to 24 consecutive bases of region 1, SEQ ID: 126. In some embodiments, oligonucleotides target a portion of MuSK transcript comprising a sequence that is identical to 25 consecutive bases of region 1, SEQ ID: 126. In some embodiments, oligonucleotides target a portion of MuSK transcript comprising a sequence that is identical to 26 consecutive bases of region 1, SEQ ID: 126. In some embodiments, oligonucleotides target a portion of MuSK transcript comprising a sequence that is identical to 27 consecutive bases of region 1, SEQ ID: 126. In some embodiments, oligonucleotides target a portion of MuSK transcript comprising a sequence that is identical to 28 consecutive bases of region 1, SEQ ID: 126. In some embodiments, oligonucleotides target a portion of MuSK transcript comprising a sequence that is identical to 29 consecutive bases of region 1, SEQ ID: 126. In some embodiments, oligonucleotides target a portion of MuSK transcript comprising a sequence that is identical to 30 consecutive bases of region 1, SEQ ID: 126.

In some embodiments, oligonucleotides target a region on the MuSK genomic sequence within or comprising at least a portion of sequence GGGGAGAAGTTCAGTACTGCCAAGGCTGCAGCCACCATCAGCATAGCAGGTAGG ATGCCCCTTCACATTTG (region 2, SEQ ID 211). In some embodiments, oligonucleotides target a portion of MUSK transcript comprising a sequence that is at least 10%, 15%, 20%, 25%, 30%, 35%, 40%, 45%, or 50% identical to region 2, SEQ ID: 211. In some embodiments, oligonucleotides target a portion of MUSK transcript comprising a sequence that is identical to at least 10, 15, 18, 19, 20, 21, 22, 23, 24, 25, 26, 27, 28, 29, or 30 consecutive bases of region 2, SEQ ID: 211. In some embodiments, oligonucleotides target a portion of MUSK transcript comprising a sequence that is identical to no more than 30 consecutive bases of region 2, SEQ ID: 211. In some embodiments, oligonucleotides target a portion of MUSK transcript comprising a sequence that is identical to at least 15 consecutive bases of region 2, SEQ ID: 211. In some embodiments, oligonucleotides target a portion of MUSK transcript comprising a sequence that is identical to 15-30 consecutive bases of region 2, SEQ ID: 211. In some embodiments, oligonucleotides target a portion of MUSK transcript comprising a sequence that is identical to 15, 16, 17, 18, 19, 20, 21, 22, 23, 24, 25, 26, 27, 28, 29, or 30 consecutive bases of region 2, SEQ ID: 211. In some embodiments, oligonucleotides target a portion of MuSK transcript comprising a sequence that is identical to 15 consecutive bases of region 2, SEQ ID: 211. In some embodiments, oligonucleotides target a portion of MuSK transcript comprising a sequence that is identical to 16 consecutive bases of region 2, SEQ ID: 211. In some embodiments, oligonucleotides target a portion of MuSK transcript comprising a sequence that is identical to 17 consecutive bases of region 2, SEQ ID: 211. In some embodiments, oligonucleotides target a portion of MuSK transcript comprising a sequence that is identical to 18 consecutive bases of region 2, SEQ ID: 211. In some embodiments, oligonucleotides target a portion of MuSK transcript comprising a sequence that is identical to 19 consecutive bases of region 2, SEQ ID: 211. In some embodiments, oligonucleotides target a portion of MuSK transcript comprising a sequence that is identical to 20 consecutive bases of region 2, SEQ ID: 211. In some embodiments, oligonucleotides target a portion of MuSK transcript comprising a sequence that is identical to 21 consecutive bases of region 2, SEQ ID: 211. In some embodiments, oligonucleotides target a portion of MuSK transcript comprising a sequence that is identical to 22 consecutive bases of region 2, SEQ ID: 211. In some embodiments, oligonucleotides target a portion of MuSK transcript comprising a sequence that is identical to 23 consecutive bases of region 2, SEQ ID: 211. In some embodiments, oligonucleotides target a portion of MuSK transcript comprising a sequence that is identical to 24 consecutive bases of region 2. SEQ ID: 211. In some embodiments, oligonucleotides target a portion of MuSK transcript comprising a sequence that is identical to 25 consecutive bases of region 2, SEQ ID: 211. In some embodiments, oligonucleotides target a portion of MuSK transcript comprising a sequence that is identical to 26 consecutive bases of region 2, SEQ ID: 211. In some embodiments, oligonucleotides target a portion of MuSK transcript comprising a sequence that is identical to 27 consecutive bases of region 2, SEQ ID: 211. In some embodiments, oligonucleotides target a portion of MuSK transcript comprising a sequence that is identical to 28 consecutive bases of region 2, SEQ ID: 211. In some embodiments, oligonucleotides target a portion of MuSK transcript comprising a sequence that is identical to 29 consecutive bases of region 2, SEQ ID: 211. In some embodiments, oligonucleotides target a portion of MuSK transcript comprising a sequence that is identical to 30 consecutive bases of region 2, SEQ ID: 211.

In some embodiments, an oligonucleotide comprises a nucleic acid strand that is complementary to a sequence within or comprising at least a portion of ACTCTGTCAGGTTTCTTCTGGGTCCATTCAAGAGAGTGTGAAAGACCGAGTGATT GACTCAAGAC (region 1, SEQ ID: 126). In some embodiments, an oligonucleotide comprises a nucleic acid strand that is complementary to a sequence that is at least 10%, 15%, 20%, 25%, 30%, 35%, 40%, 45%, or 50% identical to region 1 (SEQ ID: 126). In some embodiments, an oligonucleotide comprises a sequence that is at least 90% identical to a portion of region 1 (SEQ ID: 126) that includes at least 10, 11, 12, 13, 14, 15, 16, 17, 18, 19, 20, 21, 22, 23, 24, 25, 26, 27, 28, 29, or 30 consecutive bases of region 1 (SEQ ID: 126). In some embodiments, an oligonucleotide comprises a sequence that is at least 95% identical to a portion of region 1 (SEQ ID: 126) that includes at least 10, 11, 12, 13, 14, 15, 16, 17, 18, 19, 20, 21, 22, 23, 24, 25, 26, 27, 28, 29, or 30 consecutive bases of region 1 (SEQ ID: 126). In some embodiments, an oligonucleotide comprises a sequence that is at least 90% identical to a portion of region 1 (SEQ ID: 126) that includes at least 10 consecutive bases of region 1 (SEQ ID: 126). In some embodiments, an oligonucleotide comprises a sequence that is at least 90% identical to a portion of region 1 (SEQ ID: 126) that includes at least 11 consecutive bases of region 1 (SEQ ID: 126). In some embodiments, an oligonucleotide comprises a sequence that is at least 90% identical to a portion of region 1 (SEQ ID: 126) that includes at least 12 consecutive bases of region 1 (SEQ ID: 126). In some embodiments, an oligonucleotide comprises a sequence that is at least 90% identical to a portion of region 1 (SEQ ID: 126) that includes at least 13 consecutive bases of region 1 (SEQ ID: 126). In some embodiments, an oligonucleotide comprises a sequence that is at least 90% identical to a portion of region 1 (SEQ ID: 126) that includes at least 14 consecutive bases of region 1 (SEQ ID: 126). In some embodiments, an oligonucleotide comprises a sequence that is at least 90% identical to a portion of region 1 (SEQ ID: 126) that includes at least 15 consecutive bases of region 1 (SEQ ID: 126). In some embodiments, an oligonucleotide comprises a sequence that is at least 90% identical to a portion of region 1 (SEQ ID: 126) that includes at least 16 consecutive bases of region 1 (SEQ ID: 126). In some embodiments, an oligonucleotide comprises a sequence that is at least 90% identical to a portion of region 1 (SEQ ID: 126) that includes at least 17 consecutive bases of region 1 (SEQ ID: 126). In some embodiments, an oligonucleotide comprises a sequence that is at least 90% identical to a portion of region 1 (SEQ ID: 126) that includes at least 18 consecutive bases of region 1 (SEQ ID: 126). In some embodiments, an oligonucleotide comprises a sequence that is at least 90% identical to a portion of region 1 (SEQ ID: 126) that includes at least 19 consecutive bases of region 1 (SEQ ID: 126). In some embodiments, an oligonucleotide comprises a sequence that is at least 90% identical to a portion of region 1 (SEQ ID: 126) that includes at least 20 consecutive bases of region 1 (SEQ ID: 126). In some embodiments, an oligonucleotide comprises a sequence that is at least 90% identical to a portion of region 1 (SEQ ID: 126) that includes at least 21 consecutive bases of region 1 (SEQ ID: 126). In some embodiments, an oligonucleotide comprises a sequence that is at least 90% identical to a portion of region 1 (SEQ ID: 126) that includes at least 22 consecutive bases of region 1 (SEQ ID: 126). In some embodiments, an oligonucleotide comprises a sequence that is at least 90% identical to a portion of region 1 (SEQ ID: 126) that includes at least 23 consecutive bases of region 1 (SEQ ID: 126). In some embodiments, an oligonucleotide comprises a sequence that is at least 90% identical to a portion of region 1 (SEQ ID: 126) that includes at least 24 consecutive bases of region 1 (SEQ ID: 126). In some embodiments, an oligonucleotide comprises a sequence that is at least 90% identical to a portion of region 1 (SEQ ID: 126) that includes at least 25 consecutive bases of region 1 (SEQ ID: 126). In some embodiments, an oligonucleotide comprises a sequence that is at least 90% identical to a portion of region 1 (SEQ ID: 126) that includes at least 26 consecutive bases of region 1 (SEQ ID: 126). In some embodiments, an oligonucleotide comprises a sequence that is at least 90% identical to a portion of region 1 (SEQ ID: 126) that includes at least 27 consecutive bases of region 1 (SEQ ID: 126). In some embodiments, an oligonucleotide comprises a sequence that is at least 90% identical to a portion of region 1 (SEQ ID: 126) that includes at least 28 consecutive bases of region 1 (SEQ ID: 126). In some embodiments, an oligonucleotide comprises a sequence that is at least 90% identical to a portion of region 1 (SEQ ID: 126) that includes at least 29 consecutive bases of region 1 (SEQ ID: 126). In some embodiments, an oligonucleotide comprises a sequence that is at least 90% identical to a portion of region 1 (SEQ ID: 126) that includes at least 30 consecutive bases of region 1 (SEQ ID: 126).

In some embodiments, an oligonucleotide comprises a nucleic acid strand that is complementary to at least 10, 15, 18, 19, 20, 21, 22, 23, 24, 25, 26, 27, 28, 29, or 30 consecutive bases of region 1, SEQ ID: 126. In some embodiments, an oligonucleotide comprises a nucleic acid strand that is complementary to no more than 30 consecutive bases of region 1, SEQ ID: 126. In some embodiments, an oligonucleotide comprises a nucleic acid strand that is complementary to 15-30 consecutive bases of region 1, SEQ ID: 126.

In some embodiments, an oligonucleotide comprises a nucleic acid strand that is complementary to 15, 16, 17, 18, 19, 20, 21, 22, 23, 24, 25, 26, 27, 28, 29, or 30 consecutive bases of region 1, SEQ ID: 126. In some embodiments, an oligonucleotide comprises a nucleic acid strand that is complementary to 15 consecutive bases of region 1, SEQ ID: 126. In some embodiments, an oligonucleotide comprises a nucleic acid strand that is complementary to 16 consecutive bases of region 1, SEQ ID: 126. In some embodiments, an oligonucleotide comprises a nucleic acid strand that is complementary to 17 consecutive bases of region 1, SEQ ID: 126. In some embodiments, an oligonucleotide comprises a nucleic acid strand that is complementary to 18 consecutive bases of region 1, SEQ ID: 126. In some embodiments, an oligonucleotide comprises a nucleic acid strand that is complementary to 19 consecutive bases of region 1, SEQ ID: 126. In some embodiments, an oligonucleotide comprises a nucleic acid strand that is complementary to 20 consecutive bases of region 1, SEQ ID: 126. In some embodiments, an oligonucleotide comprises a nucleic acid strand that is complementary to 21 consecutive bases of region 1, SEQ ID: 126. In some embodiments, an oligonucleotide comprises a nucleic acid strand that is complementary to 22 consecutive bases of region 1, SEQ ID: 126. In some embodiments, an oligonucleotide comprises a nucleic acid strand that is complementary to 23 consecutive bases of region 1, SEQ ID: 126. In some embodiments, an oligonucleotide comprises a nucleic acid strand that is complementary to 24 consecutive bases of region 1, SEQ ID: 126. In some embodiments, an oligonucleotide comprises a nucleic acid strand that is complementary to 25 consecutive bases of region 1, SEQ ID: 126. In some embodiments, an oligonucleotide comprises a nucleic acid strand that is complementary to 26 consecutive bases of region 1, SEQ ID: 126. In some embodiments, an oligonucleotide comprises a nucleic acid strand that is complementary to 27 consecutive bases of region 1, SEQ ID: 126. In some embodiments, an oligonucleotide comprises a nucleic acid strand that is complementary to 28 consecutive bases of region 1, SEQ ID: 126. In some embodiments, an oligonucleotide comprises a nucleic acid strand that is complementary to 29 consecutive bases of region 1, SEQ ID: 126. In some embodiments, an oligonucleotide comprises a nucleic acid strand that is complementary to 30 consecutive bases of region 1, SEQ ID: 126.

In some embodiments, an oligonucleotide has a length of 30 bases. In some embodiments, an oligonucleotide has a length of 29 bases. In some embodiments, an oligonucleotide has a length of 28 bases. In some embodiments, an oligonucleotide has a length of 27 bases. In some embodiments, an oligonucleotide has a length of 26 bases. In some embodiments, the oligonucleotide has a length of 25 bases. In some embodiments, the oligonucleotide has a length of 24 bases. In some embodiments, the oligonucleotide has a length of 23 bases. In some embodiments, the oligonucleotide has a length of 22 bases. In some embodiments, the oligonucleotide has a length of 21 bases. In some embodiments, the oligonucleotide has a length of 20 bases. In some embodiments, the oligonucleotide has a length of 19 bases. In some embodiments, the oligonucleotide has a length of 18 bases. In some embodiments, the oligonucleotide has a length of 17 bases. In some embodiments, the oligonucleotide has a length of 16 bases. In some embodiments, the oligonucleotide has a length of 15 bases.

In some embodiments, the oligonucleotide has a length of less than about 50 bases. In some embodiments, the oligonucleotide has a length of less than about 40 bases. In some embodiments, the oligonucleotide has a length of less than about 30 bases. In some embodiments, the oligonucleotide has a length of more than about 10 bases. In some embodiments, the oligonucleotide has a length of more than about 15 bases. In some embodiments, the oligonucleotide has a length of more than about 20 bases. In some embodiments, the base sequence of the oligonucleotide comprises from 5′ to 3″:

| SEQ ID | Oligo ID | 5′ to 3′ Sequence |
|---|---|---|
| 1 | Bld1 | GCTAGGGTGGTCTTTTAGAAATGCA |
| 2 | Bld2 | GGTCAAGCTAGGGTGGTCTTTTAGA |
| 3 | Bld3 | CTGCAGGAAATGGTCAAGCTAGGGT |
| 4 | Bld4 | GAAGTGGTGAGTGACGCTCCTGCAG |
| 5 | Bld5 | GTTAGGAAGACAGAAGTGGTGAGTG |
| 6 | Bld6 | ATCCTGGCAAAAACTGTTAGGAAGA |
| 7 | Bld7 | GTGGGATTCAGGAGCCCGCAGGATC |
| 8 | Bld8 | GGTGACATTGTGGGATTCAGGAGCC |
| 9 | Bld9 | GGAGCCAAAGGTGACATTGTGGGAT |
| 10 | Bld10 | GGTCACAAAGGAGCCAAAGGTGACA |
| 11 | Bld11 | ACAGTGCAGGGTCACAAAGGAGCCA |
| 12 | Bld12 | CTGTTGCTGTACAGTGCAGGGTCAC |
| 13 | Bld13 | GGGACAGGAATGCCTGTTGCTGTAC |
| 14 | Bld14 | CAGGTGATGGTGGGGACAGGAATGC |
| 15 | Bld15 | CCGTTTTCAATCCAGGTGATGGTGG |
| 16 | Bld16 | TGACACTCACAGCATTTCCGTTTTC |
| 17 | Bld17 | AAGTCCCCACACACATGACACTCAC |
| 18 | Bld18 | GGTCTTCCCCAGACAAGTCCCCACA |
| 19 | Bld19 | ACTATGTCAGTAGATTTGAAGGGAA |
| 20 | Bld20 | TCCCACTATACTATGTCAGTAGATT |
| 21 | Bld21 | TCAGTCAAGGATTTCCCACTATACT |
| 22 | Bld22 | AAAAGAACTCAGTCAAGGATTTCCC |
| 23 | Bld23 | GTAAAGGAAAATAAAAGAACTCAGT |
| 24 | Bld24 | AACCTGACAGAGTAAAGGAAAATAA |
| 25 | Bld25 | GGACCCAGAAGAAACCTGACAGAGT |
| 26 | Bld26 | CACTCTCTTGAATGGACCCAGAAGA |
| 27 | Bld27 | CACTCGGTCTTTCACACTCTCTTGA |
| 28 | Bld28 | GTCTTGAGTCAATCACTCGGTCTTT |
| 29 | Bld29 | GATAAACAGCTGCAGTCTTGAGTCA |
| 30 | Bld30 | AGTCCTGGCTTGGTGATAAACAGCT |
| 31 | Bld31 | ATGTGTAGAGTCCTGGCTTGGTGAT |
| 32 | Bld32 | GTAGCTATGCATGTGTAGAGTCCTG |
| 33 | Bld33 | TGCTTATTGGTAGCTATGCATGTGT |
| 34 | Bld34 | ACTTCTCCCCATGCTTATTGGTAGC |
| 35 | Bld35 | CCTTGGCAGTACTGAACTTCTCCCC |
| 36 | Bld36 | CCTGCTATGCTGATGGTGGCTGCAG |
| 37 | Bld37 | GGGCATCCTACCTGCTATGCTGATG |
| 38 | Bld38 | GCAAATGTGAAGGGGCATCCTACCT |
| 127 | Bld51 | TTGAATGGACCCAGAAGAAA |
| 128 | Bld52 | CTTGAATGGACCCAGAAGAA |
| 129 | Bld53 | TCTTGAATGGACCCAGAAGA |
| 130 | Bld54 | CTCTTGAATGGACCCAGAAG |
| 131 | Bld55 | TCTCTTGAATGGACCCAGAA |
| 132 | Bld56 | CTCTCTTGAATGGACCCAGA |
| 133 | Bld57 | ACTCTCTTGAATGGACCCAG |
| 134 | Bld58 | CACTCTCTTGAATGGACCCA |
| 135 | Bld59 | ACACTCTCTTGAATGGACCC |
| 136 | Bld60 | CACACTCTCTTGAATGGACC |
| 137 | Bld61 | TCACACTCTCTTGAATGGAC |
| 138 | Bld62 | TTCACACTCTCTTGAATGGA |
| 139 | Bld63 | TTTCACACTCTCTTGAATGG |
| 140 | Bld64 | CTTTCACACTCTCTTGAATG |
| 141 | Bld65 | TCTTTCACACTCTCTTGAAT |
| 142 | Bld66 | GTCTTTCACACTCTCTTGAA |
| 159 | Bld25-1 | CCAGAAGAAACCTGACAGAGTAAAG |
| 160 | Bld25-2 | ACCCAGAAGAAACCTGACAGAGTAA |
| 161 | Bld25-3 | ATGGACCCAGAAGAAACCTGACAGA |
| 162 | Bld25-4 | GAATGGACCCAGAAGAAACCTGACA |
| 163 | Bld25-5 | CTTGAATGGACCCAGAAGAAACCTG |
| 164 | Bld26-1 | CTCTTGAATGGACCCAGAAGAAACC |
| 165 | Bld26-2 | CTCTCTTGAATGGACCCAGAAGAAA |
| 166 | Bld26-3 | CACACTCTCTTGAATGGACCCAGAA |
| 167 | Bld26-4 | TTCACACTCTCTTGAATGGACCCAG |
| 177 | Bld25-A | ACCCAGAAGAAACCTGACAGAGT |
| 178 | Bld25-B | CCAGAAGAAACCTGACAGAGT |
| 179 | Bld25-C | GGACCCAGAAGAAACCTGACAGA |
| 180 | Bld25-D | ACCCAGAAGAAACCTGACAGA |
| 181 | Bld25-E | GGACCCAGAAGAAACCTGACA |
| 182 | Bld25-5-A | GAATGGACCCAGAAGAAACCTGA |
| 183 | Bld25-5-B | ATGGACCCAGAAGAAACCTGA |
| 184 | Bld25-5-C | TTGAATGGACCCAGAAGAAACCT |
| 185 | Bld25-5-D | GAATGGACCCAGAAGAAACCT |
| 186 | Bld25-5-E | TTGAATGGACCCAGAAGAAAC |
| 187 | Bld26-2-A | CTCTTGAATGGACCCAGAAGAAA |
| 188 | Bld26-2-B | CTTGAATGGACCCAGAAGAAA |
| 189 | Bld26-2-C | CTCTCTTGAATGGACCCAGAAGA |
| 190 | Bld26-2-D | CTCTCTTGAATGGACCCAGAA |
| 191 | Bld26-B | CTCTTGAATGGACCCAGAAGA |
| 192 | Bld26-C | CACTCTCTTGAATGGACCCAGAA, or |
| 193 | Bld26-D | CACTCTCTTGAATGGACCCAG |

In some embodiments, the oligonucleotide is complementary to a nucleotide sequence that is at least 90% identical to any one of SEQ ID NOs: 39-76 and 212-253.

In some embodiments, the oligonucleotides comprise one or more types of base modifications, sugar modification, and internucleotidic linkage modifications. In some embodiments, the oligonucleotides comprise non-natural sugar moieties, or non-natural internucleotidic linkages, or both.

In some embodiments, the oligonucleotides comprise internucleotidic linkage modifications. In some embodiments, the internucleotidic linkages of the oligonucleotide comprises natural phosphate, phosphorothioate, or phosphodithioate linkages. In some embodiments, each internucleotidic linkages of the oligonucleotide is a phosphorothioate linkage. In some embodiments, each internucleotidic linkage of the oligonucleotide is a natural phosphate linkage. In some embodiments, oligonucleotide comprises at least one natural phosphate linkage and at least one phosphodithioate linkage. In some embodiments, at least 50%, 60%, 70%, 80%, 90%, 94%, or 95% of internucleotidic linkages of an oligonucleotide are phosphodithioate linkages. In some embodiments, at least 50%, 60%, 70%, 80%, 90%, 94%, or 95% of internucleotidic linkages of an oligonucleotide are natural phosphate linkages.

In some embodiments, the oligonucleotides comprise sugar modification. In some embodiments, the modified sugar moiety has a 2′-modification. In some embodiments, the modified sugar moiety comprises a bicyclic sugar modification. In some embodiments, the modified sugar moiety comprises a 2′-modification, wherein a 2′-modification is 2′-OR¹, wherein R¹ is optionally substituted C₁₋₆ alkyl. In some embodiments, the modified sugar moiety comprises a 2′-modification, wherein a 2′-modification is 2′-MOE. In some embodiments, the modified sugar moiety comprises a 2′-modification, wherein a 2′-modification is 2′-OMe. In some embodiments, each sugar of the oligonucleotide is a 2′-MOE modified sugar. In some embodiments, an oligonucleotide comprises 2′-OH sugar (RNA sugar). In some embodiments, an oligonucleotide comprise 2′-H sugar (DNA sugar). In some embodiments, an oligonucleotide comprises 2′-MOE sugar. In some embodiments, an oligonucleotide comprises 2′-OMe sugar. In some embodiments, an oligonucleotide comprises 2′-MOE, 2′-OMe, 2′-OH, 2′-H sugar, or any combination thereof. In some embodiments, an oligonucleotide comprises at least one 2′-MOE sugar and at least one 2′-OH sugar (RNA sugar). In some embodiments, an oligonucleotide comprises at least one 2′-MOE sugar and at least one 2′-H sugar (DNA sugar).

In some embodiments, the oligonucleotide has the structure from 5′ to 3′ of:

| SEQ- | Oligo | |
|---|---|---|
| ID | ID | Oligonucleotide structure, 5′ to 3′ |
| 78 | Bld1 | G*C*T *A*G*G *G*T*G *G*T*C *T*T*T *T*A*G *A*A*A *T*G*C *A |
| 79 | Bld2 | G*G*T *C*A*A *G*C*T *A*G*G *G*T*G *G*T*C *T*T*T *T*A*G *A |
| 80 | Bld3 | C*T*G *C*A*G *G*A*A *A*T*G *G*T*C *A*A*G *C*T*A *G*G*G *T |
| 81 | Bld4 | G*A*A *G*T*G *G*T*G *A*G*T *G*A*C *G*C*T *C*C*T *G*C*A *G |
| 82 | Bld5 | G*T*T *A*G*G *A*A*G *A*C*A *G*A*A *G*T*G *G*T*G *A*G*T *G |
| 83 | Bld6 | A*T*C *C*T*G *G*C*A *A*A*A *A*C*T *G*T*T *A*G*G *A*A*G *A |
| 84 | Bld7 | G*T*G *G*G*A *T*T*C *A*G*G *A*G*C *C*C*G *C*A*G *G*A*T *C |
| 85 | Bld8 | G*G*T *G*A*C *A*T*T *G*T*G *G*G*A *T*T*C *A*G*G *A*G*C *C |
| 86 | Bld9 | G*G*A *G*C*C *A*A*A *G*G*T *G*A*C *A*T*T *G*T*G *G*G*A *T |
| 87 | Bld10 | G*G*T *C*A*C *A*A*A *G*G*A *G*C*C *A*A*A *G*G*T *G*A*C *A |
| 88 | Bld11 | A*C*A *G*T*G *C*A*G *G*G*T *C*A*C *A*A*A *G*G*A *G*C*C *A |
| 89 | Bld12 | C*T*G *T*T*G *C*T*G *T*A*C *A*G*T *G*C*A *G*G*G *T*C*A *C |
| 90 | Bld13 | G*G*G *A*C*A *G*G*A *A*T*G *C*C*T *G*T*T *G*C*T *G*T*A *C |
| 91 | Bld14 | C*A*G *G*T*G *A*T*G *G*T*G *G*G*G *A*C*A *G*G*A *A*T*G *C |
| 92 | Bld15 | C*C*G *T*T*T *T*C*A *A*T*C *C*A*G *G*T*G *A*T*G *G*T*G *G |
| 93 | Bld16 | T*G*A *C*A*C *T*C*A *C*A*G *C*A*T *T*T*C *C*G*T *T*T*T *C |
| 94 | Bld17 | A*A*G *T*C*C *C*C*A *C*A*C *A*C*A *T*G*A *C*A*C *T*C*A *C |
| 95 | Bld18 | G*G*T *C*T*T *C*C*C *C*A*G *A*C*A *A*G*T *C*C*C *C*A*C *A |
| 96 | Bld19 | A*C*T *A*T*G *T*C*A *G*T*A *G*A*T *T*T*G *A*A*G *G*G*A *A |
| 97 | Bld20 | T*C*C *C*A*C *T*A*T *A*C*T *A*T*G *T*C*A *G*T*A *G*A*T *T |
| 98 | Bld21 | T*C*A *G*T*C *A*A*G *G*A*T *T*T*C *C*C*A *C*T*A *T*A*C *T |
| 99 | Bld22 | A*A*A *A*G*A *A*C*T *C*A*G *T*C*A *A*G*G *A*T*T *T*C*C *C |
| 100 | Bld23 | G*T*A *A*A*G *G*A*A *A*A*T *A*A*A *A*G*A *A*C*T *C*A*G *T |
| 101 | Bld24 | A*A*C *C*T*G *A*C*A *G*A*G *T*A*A *A*G*G *A*A*A *A*T*A *A |
| 102 | Bld25 | G*G*A *C*C*C *A*G*A *A*G*A *A*A*C *C*T*G *A*C*A *G*A*G *T |
| 103 | Bld26 | C*A*C *T*C*T *C*T*T *G*A*A *T*G*G *A*C*C *C*A*G *A*A*G *A |
| 104 | Bld27 | C*A*C *T*C*G *G*T*C *T*T*T *C*A*C *A*C*T *C*T*C *T*T*G *A |
| 105 | Bld28 | G*T*C *T*T*G *A*G*T *C*A*A *T*C*A *C*T*C *G*G*T *C*T*T *T |
| 106 | Bld29 | G*A*T *A*A*A *C*A*G *C*T*G *C*A*G *T*C*T *T*G*A *G*T*C *A |
| 107 | Bld30 | A*G*T *C*C*T *G*G*C *T*T*G *G*T*G *A*T*A *A*A*C *A*G*C *T |
| 108 | Bld31 | A*T*G *T*G*T *A*G*A *G*T*C *C*T*G *G*C*T *T*G*G *T*G*A *T |
| 109 | Bld32 | G*T*A *G*C*T *A*T*G *C*A*T *G*T*G *T*A*G *A*G*T *C*C*T *G |
| 110 | Bld33 | T*G*C *T*T*A *T*T*G *G*T*A *G*C*T *A*T*G *C*A*T *G*T*G *T |
| 111 | Bld34 | A*C*T *T*C*T *C*C*C *C*A*T *G*C*T *T*A*T *T*G*G *T*A*G *C |
| 112 | Bld35 | C*C*T *T*G*G *C*A*G *T*A*C *T*G*A *A*C*T *T*C*T *C*C*C *C |
| 113 | Bld36 | C*C*T *G*C*T *A*T*G *C*T*G *A*T*G *G*T*G *G*C*T *G*C*A *G |
| 114 | Bld37 | G*G*G *C*A*T *C*C*T *A*C*C *T*G*C *T*A*T *G*C*T *G*A*T *G |
| 115 | Bld38 | G*C*A *A*A*T *G*T*G *A*A*G *G*G*G *C*A*T *C*C*T *A*C*C *T |
| 143 | Bld51 | T*T*G*A*A*T*G*G*A*C*C*C*A*G*A*A*G*A*A*A |
| 144 | Bld52 | C*T*T*G*A*A*T*G*G*A*C*C*C*A*G*A*A*G*A*A |
| 145 | Bld53 | T*C*T*T*G*A*A*T*G*G*A*C*C*C*A*G*A*A*G*A |
| 146 | Bld54 | C*T*C*T*T*G*A*A*T*G*G*A*C*C*C*A*G*A*A*G |
| 147 | Bld55 | T*C*T*C*T*T*G*A*A*T*G*G*A*C*C*C*A*G*A*A |
| 148 | Bld56 | C*T*C*T*C*T*T*G*A*A*T*G*G*A*C*C*C*A*G*A |
| 149 | Bld57 | A*C*T*C*T*C*T*T*G*A*A*T*G*G*A*C*C*C*A*G |
| 150 | Bld58 | C*A*C*T*C*T*C*T*T*G*A*A*T*G*G*A*C*C*C*A |
| 151 | Bld59 | A*C*A*C*T*C*T*C*T*T*G*A*A*T*G*G*A*C*C*C |
| 152 | Bld60 | C*A*C*A*C*T*C*T*C*T*T*G*A*A*T*G*G*A*C*C |
| 153 | Bld61 | T*C*A*C*A*C*T*C*T*C*T*T*G*A*A*T*G*G*A*C |
| 154 | Bld62 | T*T*C*A*C*A*C*T*C*T*C*T*T*G*A*A*T*G*G*A |
| 155 | Bld63 | T*T*T*C*A*C*A*C*T*C*T*C*T*T*G*A*A*T*G*G |
| 156 | Bld64 | C*T*T* *C*A*C*A*C*T*C*T*C*T*T*G*A*A*T*G |
| 157 | Bld65 | T*C*T*T*T*C*A*C*A*C*T*C*T*C*T*T*G*A*A*T |
| 158 | Bld66 | G*T*C*T*T*T*C*A*C*A*C*T*C*T*C*T*T*G*A*A |
| 168 | Bld25-1 | C*C*A*G*A*A*G*A*A*A*C*C*T*G*A*C*A*G*A*G*T*A*A*A*G* |
| 169 | Bld25-2 | A*C*C*C*A*G*A*A*G*A*A*A*C*C*T*G*A*C*A*G*A*G*T*A*A* |
| 170 | Bld25-3 | A*T*G*G*A*C*C*C*A*G*A*A*G*A*A*A*C*C*T*G*A*C*A*G*A* |
| 171 | Bld25-4 | G*A*A*T*G*G*A*C*C*C*A*G*A*A*G*A*A*A*C*C*T*G*A*C*A* |
| 172 | Bld25-5 | C*T*T*G*A*A*T*G*G*A*C*C*C*A*G*A*A*G*A*A*A*C*C*T*G* |
| 173 | Bld26-1 | C*T*C*T*T*G*A*A*T*G*G*A*C*C*C*A*G*A*A*G*A*A*A*C*C* |
| 174 | Bld26-2 | C*T*C*T*C*T*T*G*A*A*T*G*G*A*C*C*C*A*G*A*A*G*A*A*A* |
| 175 | Bld26-3 | C*A*C*A*C*T*C*T*C*T*T*G*A*A*T*G*G*A*C*C*C*A*G*A*A* |
| 176 | Bld26-4 | T*T*C*A*C*A*C*T*C*T*C*T*T*G*A*A*T*G*G*A*C*C*C*A*G* |
| 194 | Bld25-A | A*C*C*C*A*G*A*A*G*A*A*A*C*C*T*G*A*C*A*G*A*G*T |
| 195 | Bld25-B | C*C*A*G*A*A*G*A*A*A*C*C*T*G*A*C*A*G*A*G*T |
| 196 | Bld25-C | G*G*A*C*C*C*A*G*A*A*G*A*A*A*C*C*T*G*A*C*A*G*A |
| 197 | Bld25-D | A*C*C*C*A*G*A*A*G*A*A*A*C*C*T*G*A*C*A*G*A |
| 198 | Bld25-E | G*G*A*C*C*C*A*G*A*A*G*A*A*A*C*C*T*G*A*C*A |
| 199 | Bld25-5-A | G*A*A*T*G*G*A*C*C*C*A*G*A*A*G*A*A*A*C*C*T*G*A |
| 200 | Bld25-5-B | A*T*G*G*A*C*C*C*A*G*A*A*G*A*A*A*C*C*T*G*A |
| 201 | Bld25-5-C | T*T*G*A*A*T*G*G*A*C*C*C*A*G*A*A*G*A*A*A*C*C*T |
| 202 | Bld25-5-D | G*A*A*T*G*G*A*C*C*C*A*G*A*A*G*A*A*A*C*C*T |
| 203 | Bld25-5-E | T*T*G*A*A*T*G*G*A*C*C*C*A*G*A*A*G*A*A*A*C |
| 204 | Bld26-2-A | C*T*C*T*T*G*A*A*T*G*G*A*C*C*C*A*G*A*A*G*A*A*A |
| 205 | Bld26-2-B | C*T*T*G*A*A*T*G*G*A*C*C*C*A*G*A*A*G*A*A*A |
| 206 | Bld26-2-C | C*T*C*T*C*T*T*G*A*A*T*G*G*A*C*C*C*A*G*A*A*G*A |
| 207 | Bld26-2-D | C*T*C*T*C*T*T*G*A*A*T*G*G*A*C*C*C*A*G*A*A |
| 208 | Bld26-B | C*T*C*T*T*G*A*A*T*G*G*A*C*C*C*A*G*A*A*G*A |
| 209 | Bld26-C | C*A*C*T*C*T*C*T*T*G*A*A*T*G*G*A*C*C*C*A*G*A*A, or |
| 210 | Bld26-D | C*A*C*T*C*T*C*T*T*G*A*A*T*G*G*A*C*C*C*A*G. |

wherein * represents a phosphorothioate linkage, and each sugar of the oligonucleotides is a 2′-MOE modified sugar.

In some embodiments, an oligonucleotide composition comprising a mixture of two or more oligonucleotides according to any one of the embodiments.

In some embodiments, the composition comprises oligonucleotides that targets regions of the MuSK gene that correspond to positions 83776-83800 and on 83854-83878 of SEQ ID NO: 77.

In some embodiments, a pharmaceutical composition comprises a therapeutically effective amount of an oligonucleotide, and at least one pharmaceutically acceptable inactive ingredient selected from pharmaceutically acceptable diluents, pharmaceutically acceptable excipients, and pharmaceutically acceptable carriers, wherein the oligonucleotide is an oligonucleotide of any one of the embodiments.

In some embodiments, the oligonucleotides are formulated a nanocarrier. In some embodiments, the oligonucleotides are formulated in lipid nano-particles (LNPs). In some embodiments, the oligonucleotides are covalently conjugated to an additional moiety selected from lipids (for example, cholesterol), peptides, aptamers, antibodies, and sugars (for example, N-acetylgalactosamine (GalNAc). In some embodiments, the oligonucleotides are covalently conjugated to N-acetylgalactosamine (GalNAc).

In another aspect, the disclosure features, a method of altering relative amounts of MuSK spliced transcripts, the method comprising a step of administering to a subject a pharmaceutical composition that comprises or delivers a composition of any one of previous embodiments.

In some embodiments, the disclosure features the alteration of MuSK spliced transcripts being characterized in that, the ratio of MuSK transcripts containing Ig3 domain-encoding sequences to MuSK transcription containing no Ig3 domain-encoding sequences is increased. In some embodiments, the alteration of MuSK spliced transcripts being characterized in that, the ratio of MuSK transcripts containing Ig3 domain-encoding sequences to MuSK transcription containing no Ig3 domain-encoding sequences decreases. In some embodiments, the alteration of MuSK spliced transcripts being characterized in that, MuSK transcripts containing Ig3 domain-encoding sequences decreases and level of total MuSK transcripts remains substantially the same.

In some embodiments, the alteration of MuSK spliced transcripts is characterized in that, the level of MuSK transcripts including exons 6 and 7 decreases or level of MuSK protein forms including sequences encoded by exons 6 and 7 decreases, or both.

In some embodiments, the alteration of MuSK spliced transcripts being characterized in that, level of MuSK transcripts including exons 6 and 7 is increased or level of MuSK protein forms including sequences encoded by exons 6 and 7 is increased, or both. In some embodiments, the alteration of MuSK spliced transcripts is characterized in that, the level of MuSK transcripts including exons 3 and 4 decreases or level of MuSK protein forms including sequences encoded by exons 3 and 4 decreases, or both. In some embodiments, the alteration of MuSK spliced transcripts is characterized in that, the level of MuSK transcripts including exons 3 and 4 is increased or level of MuSK protein forms including sequences encoded by exons 3 and 4 is increased, or both.

In some embodiments, the alteration of MuSK spliced transcripts being characterized in that, the level of MuSK transcripts including exons 3 and 4 remains substantially the same, or level of MuSK protein forms including sequences encoded by exons 3 and 4 remains substantially the same, or both. In some embodiments, the alteration of MuSK spliced transcripts is characterized that, the level of MuSK transcripts including exons 3 and 4 remains substantially the same, or level of MuSK protein forms including sequences encoded by exons 3 and 4 remains substantially the same, or both; and level of MuSK transcripts including exons 6 and 7 decreases or level of MuSK protein forms including sequences encoded by exons 6 and 7 decreases, or both.

In another aspect, the disclosure features, a method of treating a subject suffering from one or more features of neurodegenerative diseases, the method comprising a step of administering to a subject a pharmaceutical composition that comprises or delivers a composition of any one of the previous embodiments.

In another aspect, the disclosure features, a method of increasing neurogenesis, the method comprising a step of administering to a subject a pharmaceutical composition that comprises or delivers a composition of any one of the previous embodiments.

In another aspect, the disclosure features, a method of treating a subject suffering from one or more features of neuromuscular dysfunction or a muscular dystrophy, the method comprising a step of administering to a subject a pharmaceutical composition that comprises or delivers a composition of any one of the previous embodiments.

In another aspect, the disclosure features, a method of increasing muscle regeneration and/or muscle growth, the method comprising a step of administering to a subject a pharmaceutical composition that comprises or delivers a composition of any one of the previous embodiments.

In another aspect, the disclosure features, a method of treating muscle fibrosis, the method comprising a step of administering to a subject a pharmaceutical composition that comprises or delivers a composition of any one of the previous embodiments.

In some embodiments, the subject is at risk of, or afflicted with, a disease or disorder selected from the group consisting of: neuromuscular dysfunction, neurodegenerative disorder, cardiac disorder, and diseases characterized by muscle wasting. In some embodiments, the neurodegenerative disorder is selected from the group consisting of Alzheimer's Disease (AD), Parkinson's disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy, Hippocampal Sclerosis, Niemann Pick Type C, Diabetes-mediated hippocampal neuronal loss, brain injury (e.g., traumatic and/or anoxic brain injury), and Huntington's disease. In some embodiments, the neurodegenerative disease is Alzheimer's Disease (AD).

In some embodiments, the neuromuscular dysfunction is a muscular dystrophy selected from the group consisting of: Becker, Congenital, Distal, Duchenne, Emery-Dreifuss, Facioscapulohumeral, Limb-girdle, Myotonic, and Oculo-pharyngeal muscular dystrophy. In another aspect, the disclosure features,

In some embodiments, the cardiac disorder is myocardial infarction or cardiomyopathy. In some embodiments, the subject is in need of enhanced muscle regeneration and/or growth following a condition selected from the group consisting of: surgery, trauma and prolonged immobilization. In some embodiments, the prolonged immobilization results from bed-rest or casting. In some embodiments, the subject is at risk of, or afflicted with, sarcopenia. In some embodiments, the subject is at risk of, or afflicted with, muscle fibrosis resulting from a disease or condition selected from the group consisting of: trauma, heritable disease, muscle disorder, and aging. In some embodiments, the trauma is the result of a condition selected from the group consisting of: radiation treatment, crush injury, laceration, and amputation.

In some embodiments, the heritable disease or muscle disorder selected from the group consisting of: Congenital Muscular Dystrophy, Duchenne Muscular Dystrophy, Becker's Muscular Dystrophy: Amyotrophic Lateral Sclerosis (ALS), and age-associate sarcopenia.

In some embodiments, the composition is delivered to the CNS. In some embodiments, the composition is delivered to the cerebrospinal fluid. In some embodiments, the composition is delivered to the muscle. In some embodiments, the composition is delivered to the liver. In some embodiments, the composition can be formulated for systemic or localized administration. In some embodiments, the composition is formulated for delivery by a route selected from intravenous injection, intravenous infusion, intramuscular injection, intrathecal administration, oral administration, buccal administration, inhalation, nasal administration, topical administration, ophthalmic administration or otic administration.

In some embodiments, the composition is formulated for delivery by intramuscular administration. In some embodiments, the composition is formulated for delivery by intravenous administration. In some embodiments, the composition is formulated for delivery by oral administration.

### BRIEF DESCRIPTION OF THE DRAWING

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1 is a schematic representation of an interaction between BMP and Ig3 domain of MuSK and its implications in BMP signaling and neurogenesis and cognition.

FIG. 2 is a schematic representation of the full-length and MuSKΔIg3 transcripts and encoded proteins and exemplary primer design to selectively detect total MuSK and full-length MuSK transcripts.

FIG. 3 shows relative MuSK expression measured by qPCR using Taqman or SYBR green technology in LHCN-M2 cells.

FIG. 4 is a schematic representation of “region 1” and “region 2” of exon 7 of MuSK transcript.

FIG. 5 shows an alignment of the ASOs Bld1-Bld18 on the genetic sequence of MuSK.

FIG. 6 shows microscopic acquisitions of LHCN-M2 24 h after being transfected with the ASOs Bld1-Bld4, Bld6, Bld7, Bld9, Bld11, Bld12, Bld13, Bld14, Bld15, Bld16, Bld17, and untreated control at 100 nM (scale 100 nm).

FIG. 7 shows relative MuSK expression measured by qPCR of LHCN-M2 cells transfected with ASOs Bld1-Bld18 at 50 and 100 nM. Panel A shows total MuSK expression (i.e., of MuSK34, a primer spanning exon/exon junction 3-4) and Panel B shows expression of MuSK exons 6-7 (i.e., using MuSK67, a primer spanning exon/exon junction 6-7). All samples are normalized to the housekeeping genes GAPDH and YWHAZ and to controls.

FIG. 8 shows alignment of the ASOs Bld1-Bld18 on the genetic sequence of MuSK and their effect on MuSK expression. Green: no effect or increase in MuSK expression (MuSK expression comprised between 90 and 300% of the untreated condition). Orange: moderate decrease in MuSK expression (MuSK expression comprised between 40 and 70% of the untreated condition). Red: high decrease in MuSK expression (MuSK expression below 40% of the untreated condition). Red areas indicate regions important for the expression of MuSK where ASOs induced a decrease of MuSK67>80%.

FIG. 9 shows an alignment of the ASOs Bld19-Bld38 on the genetic sequence of MuSK.

FIG. 10 shows microscopic acquisitions of LHCN-M2 24 h after being transfected with the ASOs Bld 19-22, Bld29-34, and untreated control at 100 nM (scale 100 nm).

FIG. 11 shows relative MuSK expression measured by qPCR of LHCN-M2 cells transfected with ASOs (control, Bld19-Bld28) at 50 and 100 nM. Panel A shows MuSK34 expression and panel B shows MuSK67 expression. All samples are normalized to the housekeeping genes GAPDH and YWHAZ and to controls.

FIG. 12 shows the alignment of the ASOs Bld19-Bld38 on the genetic sequences and their effect on MuSK expression. Green: no effect or increase in MuSK expression (MuSK expression comprised between 90 and 300% of the untreated condition). Orange: moderate decrease in MuSK expression (MuSK expression comprised between 40 and 70% of the untreated condition). Red: high decrease in MuSK expression (MuSK expression below 40% of the untreated condition). Red areas indicate regions important for the expression of MuSK where ASOs induced a decrease of MuSK67>80%. Purple areas indicate regions that can be targeted to selectively inhibit MuSK67 expression (with lower effect on other exons).

FIG. 13 shows relative MuSK expression in response to various doses of ASOs (Bld25 (panel A), Bld26 (panel B), Bld27 (panel C), Bld28 (panel D), Bld35 (panel E), Bld38 (panel F)). MuSK34 (in blue) and MuSK67 (in red) expressions were measured by qPCR and normalized to housekeeping genes and to the controls. The estimated IC is indicated on each graph. The 5 tested doses were 2.5, 5, 25, 125, and 400 nM.

FIG. 14 shows relative MuSK expression in response to various doses of ASOs (Bld25 (panel A), Bld26 (panel B)). MuSK34 (in blue) and MuSK67 (in red) expressions were measured by qPCR and normalized to housekeeping genes and to the controls. The 4 tested doses were 5, 7.5, 12.5 and 25 nM.

FIG. 15 shows relative MuSK expression in response to combination of ASOs Bld25 and Bld26 at 12.5 nM final concentration compared to an untreated control. MuSK34 (in blue) and MuSK67 (in red) expressions were measured by qPCR and normalized to housekeeping genes and to controls.

FIG. 16 shows migration of PCR products from exon 3 to exon 9 on gel electrophoresis. cDNA of ASO-treated cells (Bld25, i.e., “hu7-10 or Bld26, i.e., “hu73”) was amplified by PCR and deposited on gel electrophoresis to be migrated (panel A). Predicted products are presented in panel B and panel B′.

FIG. 17 shows an alignment of sequences of various target portion of “region 1”. ASOs Bld25, Bld26, and Bld51-Bld66 target various portions of “region 1.”

FIG. 18 shows the relative change of gene expression of total MuSK (MuSK34) and MuSK containing Ig3 domain (MuSK67) for ASOs Bld51-Bld66 at a concentration of 12.5 nM (Panel A) and at a concentration of 100 nM (Panel B).

FIG. 19 shows an alignment of ASOs Bld25-1, Bld25-2, Bld25, Bld25-3, Bld25-4, BLd25-5, Bld26-1, Bld26-2, Bld26, Bld26-3, Bld26-4, Bld27, Bld28, Bld35, Bld 38 on various target portions of “region 1” and “region 2” of exon 7 of human MuSK.

FIG. 20 shows MuSK gene expression analyzed in qPCR. Panel A shows relative gene expression of MuSKIg3 (MuSK67) and Panel B shows relative gene expression of total MuSK (MuSK34).

FIG. 21 shows MuSK gene expression from only the ASOs which showed relative expression of MuSK67 of less than 50% and total MuSK (MuSK34) of greater than 60% compared to the control. Panel A shows relative gene expression of MuSKIg3 (MuSK67) and Panel B shows relative gene expression of total MuSK (MuSK34).

FIG. 22 shows a comparison of Bld25-5 to Bld25 after transfection with ASO for 24 hours (Panels A and B) and 48 hours (Panels C and D).

FIG. 23 shows an alignment of ASOs Bld25, Bld25-A, Bld25-B, Bld25-C, Bld25-D, Bld25-E, Bld25-5, Bld25-5-A, Bld25-5-B, Bld25-5-C, Bld25-5-D, Bld25-5-E, Bld26-2, Bld26-2-A, Bld26-2-B, Bld26-2-C, Bld26-2-D, Bld26, Bld26-B, Bld26-C, Bld26-D, on various target portions of “region 1” of exon 7 of human MuSK.

FIG. 24 shows relative gene expression (MuSK34 and MuSK67) for ASOs Bld25-A, Bld25-B, Bld25-C, Bld25-D, and Bld25-E.

FIG. 25 shows relative gene expression (MuSK34 and MuSK67) for ASOs Bld25-5-A, Bld25-5-B, Bld25-5-C, Bld25-5-D, and Bld25-5-E.

FIG. 26 shows a graphical representation of the 3 variants of MuSK RNA. Full length and ΔIg3 indicate the lengths of the full-length sequence and the sequence comprising a deletion of exon 6, 7, respectively, amplified by PCR from exon 3 to exon 9.

FIG. 27 shows the gel migration of pcr products from cells treated with Bld25 and Bld25-5 and intensity of bands from Bld25 and Bld25-5 products.

FIG. 28 shows that the sequence of the band lower band positioned, where Δ6,7 variant 1 (687 bases) was expected, was indeed the sequence of this splice variant.

FIG. 29 shows images of HCN-M2 cells being treated for 48 h with 10 nM of siRNA against MuSK and were then stained for MuSK protein on days 2, 3, 4, and 5 following siRNA treatment.

### DEFINITIONS

For convenience, the meaning of some terms and phrases used in the specification, examples, and appended claims, are provided below. Unless stated otherwise, or implicit from context, the following terms and phrases include the meanings provided below.

These definitions are provided to aid in describing particular embodiments, and are not intended to limit the claimed invention, because the scope of the invention is limited only by the claims.

Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

About or Approximately: The term “about” or “approximately”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent (e.g., an agonizing agent) that is, or is included in or otherwise delivered by, the composition. Those of ordinary skill in the art will be aware of a variety of routes that may, in appropriate circumstances, be utilized for administration to a subject, for example a human. For example, in some embodiments, administration may be ocular, oral, buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, an agent (e.g., an agonizing agent) is delivered to the central nervous system (CNS), e.g., delivered via intracerebroventricular administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form: in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer: in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Agonist: Those skilled in the art will appreciate that the term “agonist” may be used to refer to an agent (i.e., an “agonizing agent”), condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity: In some embodiments, an agonist may be direct (in which case it exerts its influence directly upon its target): in some embodiments, an agonist may be indirect (in which case it exerts its influence by other than binding to its target: e.g., by interacting with a regulator of the target, so that level or activity of the target is altered). In some embodiments, an agonist is a binding agent that is a protein (e.g., an antibody) or a nucleic acid (e.g., an antisense oligonucleotide) that binds a target (e.g., a protein or nucleic acid) so that level, form, and/or or activity of the target is altered. In some embodiments, the altered level, form and/or activity is an increased level of altered protein expressed from the target nucleic acid sequence. Those skilled in the art, reading the present disclosure, will appreciate that, in some embodiments, an agonizing agent may bind to (and potentially agonize) a binding target, which binding causes an increase in level or activity of a further agonized target. To give a specific example, in some embodiments, an agonizing agent that binds to a nucleic acid target may alter level and/or activity of that target, and in some specific embodiments may agonize an activity of that nucleic acid target (e.g., by increasing its modification, splicing, 5′ cap formation, and/or 3′ end formation, transport, and/or translation, etc, so that a level of a desired product—e.g., mRNA, is increased) and/or may agonize a downstream target, such as a polypeptide encoded by such nucleic acid target. To give one particular such example, in some embodiments, an agonizing agent may be or comprise an oligonucleotide that binds to a primary transcript and alters its splicing pattern so that level and/or activity of a particular spliced form (e.g., mature mRNA) is increased, which may, in turn achieved increased level of a product (e.g., a polypeptide) that is or is encoded by such particular spliced form.

Antagonist: Those skilled in the art will appreciate that the term “antagonist”, as used herein, may be used to refer to an agent (i.e., an “antagonizing agent”), condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target): in some embodiments, an antagonist may be indirect (in which case it exerts its influence by other than binding to its target: e.g., by interacting with a regulator of the target, so that level or activity of the target is altered). In some embodiments, an antagonist is binding agent that is a protein (e.g., an antibody) or a nucleic acid (e.g., an antisense oligonucleotide) that binds a target (e.g., a protein or nucleic acid) so that the level, form, and/or activity of the target is altered. In some embodiments, the altered level, form and/or activity is a decreased level of altered protein expressed from the target nucleic acid sequence. Those skilled in the art, reading the present disclosure, will appreciate that, in some embodiments, an antagonizing agent may bind to (and potentially antagonize) a binding target, which binding causes a decrease in level or activity of a further antagonized target. To give a specific example, in some embodiments, an antagonizing agent that binds to a nucleic acid target may alter level and/or activity of that target, and in some specific embodiments may antagonize an activity of that nucleic acid target (e.g., by decreasing its modification, splicing, 5′ cap formation, and/or 3′ end formation, transport, and/or translation, etc, so that a level of an undesired product—e.g., mRNA, is suppressed) and/or may antagonize a downstream target, such as a polypeptide encoded by such nucleic acid target. To give one particular such example, in some embodiment, an antagonizing agent may be or comprise an oligonucleotide that binds to a primary transcript and alters its splicing pattern so that level and/or activity of a particular spliced form (e.g., mature mRNA) is suppressed, which may, in turn achieved decreased level of a product (e.g., a polypeptide) that is or is encoded by such particular spliced form.

Antibody agent: As used herein, the term “antibody agent” refers to an agent that specifically binds to a particular antigen (e.g., that may be or comprise an epitope of a protein of interest—e.g., a MuSK protein). In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies: bi- or multi-specific antibodies (e.g., Zybodies®, etc): antibody fragments such as Fab fragments, Fab′ fragments, F(ab)2 fragments, Fd′ fragments, Fd fragments, and isolated CDRs or sets thereof: single chain Fvs: polypeptide-Fc fusions: single domain antibodies (e.g., shark single domain antibodies such as IgNAR or fragments thereof): cameloid antibodies: masked antibodies (e.g., Probodies®): Small Modular ImmunoPharmaceuticals (“SMIPs™”); single chain or Tandem diabodies (TandAb®): VHHs: Anticalins®; Nanobodies R; minibodies: BiTE®s: ankyrin repeat proteins or DARPINs®; Avimers R: DARTs: TCR-like antibodies; Adnectins®; Affilins®; Trans-Bodies®; Affibodies®; TrimerX®; MicroProteins: Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR): in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody: As used herein, the term “antibody” refers to an immunoglobulin or a derivative thereof containing an immunoglobulin domain capable of binding to an antigen (e.g., that may be or comprise an epitope of a protein of interest—e.g., a MuSK protein). The antibody can be of any species, e.g., human, rodent, rabbit, goat, chicken, etc. The antibody may be a member of any immunoglobulin class, including any of the human classes: IgG. IgM, IgA. IgD, and IgE, or subclasses thereof such as IgG1, IgG2, etc. In various embodiments of the invention the antibody is a fragment such as an Fab′, F(ab)2, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16:535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis. In some embodiments, the antibody can act as an antagonist, e.g., by binding to a target antigen, resulting in a decreased level or activity of said antigen. In some embodiments, the antibody can act as an agonist, e.g., by binding to a target antigen, resulting in an increased level or activity of said antigen.

Antisense: The term “antisense” is used herein to refer to a nucleic acid whose nucleotide sequence is complementary to part or all of a sequence found in a coding strand nucleic acid. Typically, a “coding strand” nucleic acid is one whose sequence includes part or all of an open reading frame or other stretch of residues that encodes part or all of a polypeptide. In some embodiments, the term “antisense” may particularly be used herein in reference to an oligonucleotide that binds specifically to a coding strand (i.e., to a target sequence within such coding strand). In some embodiments, a coding strand may include both coding and non-coding sequences (e.g., to give but one example, may be a transcript, such as a primary transcript, that includes both intron and exon sequences). Those skilled in the art, reading the present disclosure, will appreciate that, in some embodiments, an oligonucleotide may be considered or referred to as an “antisense” oligonucleotide when some or all of its sequence is complementary to non-coding portion(s) of its target strand. In some embodiments, an antisense oligonucleotide binds to coding sequences in a target sense strand: in some embodiments, an antisense oligonucleotide binds to non-coding sequences in a target coding strand. In some embodiments, an antisense oligonucleotide binds to both coding and non-coding sequences in a target coding strand. In some embodiments, an antisense oligonucleotide is characterized in that, when bound to its target sequence in a coding strand (e.g., a transcript), it alters post-transcriptional processing (e.g., one or more of modification, splicing, 5′ cap formation, and/or 3′ end formation, 5′ cap formation, and/or 3′ end formation, transport, and/or translation) of such coding strand. In some particular embodiments, an antisense oligonucleotide alters splicing of its target coding strand, Alternatively or additionally, in some embodiments, an antisense-coding strand complex is or can be degraded, e.g., by RNase H.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or comprise polypeptides (including, e.g., antibodies or antibody fragments). In some embodiments, binding agents are or comprise small molecules. In some embodiments, binding agents are or comprise nucleic acids (e.g., antisense oligonucleotides). In some embodiments, binding agents are aptamers. In some embodiments, binding agents are polymers: in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA (e.g., antisense oligonucleotides).

Complementary: As used herein, in accordance with its art-accepted meaning. “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence when the strands are aligned in anti-parallel orientation, the nucleotides form a complementary base pair, and the nucleic acids are complementary at that position. The percent complementarity of a first nucleic acid to a second nucleic acid may be evaluated by aligning them in antiparallel orientation for maximum complementarity over a window of evaluation, determining the total number of nt in both strands that form complementary base pairs within the window; dividing by the total number of nt within the window; and multiplying by 100. For example, AAAAAAAA and TTTGTTAT are 75% complementary since there are 12 nt in complementary base pairs out of a total of 16 nt. When computing the number of complementary nt needed to achieve a particular % complementarity; fractions are rounded to the nearest whole number. A position occupied by non-complementary nucleotides constitutes a mismatch, i.e., the position is occupied by a non-complementary base pair. In certain embodiments a window of evaluation has the length described herein for duplex portions or target portions. Complementary sequences include base-pairing of a polynucleotide comprising a first nucleotide sequence to a polynucleotide comprising a second nucleotide sequence over the entire length of both nucleotide sequences (if the same length) or over the entire length of the shorter sequence (if different lengths). Such sequences can be referred to as “perfectly complementary.” (100% complementarity) with respect to each other herein. Nucleic acids that are at least 70% complementary over a window of evaluation are considered “substantially complementary” over that window: In certain embodiments complementary nucleic acids are at least 75%, at least 80%, at least 85%, at least 90%, or at least 95% complementary over the window of evaluation. Where a first sequence is referred to as “substantially complementary” with respect to a second sequence herein, the two sequences may be perfectly complementary or they may comprise one or more unmatched bases upon hybridization, e.g., up to about 5%, 10%, 15%, 20%, or 25% unmatched bases upon hybridization, e.g., 1, 2, 3, 4, 5, or 6 mismatched base pairs upon hybridization for a duplex up to 30 base pairs, while retaining the ability to hybridize under the conditions most relevant to their intended use. It should be understood that where two oligonucleotides are designed to form, upon hybridization, one or more single stranded overhangs, such overhangs are not regarded as mismatches or unpaired nucleotides with regard to the determination of percent complementarity: For example, the two strands of a dsRNA comprising one oligonucleotide 21 nucleotides in length and another oligonucleotide 23 nucleotides in length, wherein the longer oligonucleotide comprises a sequence of 21 nucleotides that is perfectly complementary to the shorter oligonucleotide and a 2 nucleotide overhang, may be referred to as “perfectly complementary” herein. “Complementary” sequences, as used herein may include one or more non-Watson-Crick base pairs and/or base pairs formed from non-natural and other modified nucleotides, in so far as the requirements with respect to their ability to hybridize are fulfilled. Such non-Watson-Crick base pairs include, but are not limited to, G: U Wobble or Hoogsteen base pairing. Those of ordinary skill in the art are aware that guanine, cytosine, adenine, and uracil can be replaced by other bases without substantially altering the base pairing properties of a polynucleotide comprising a nucleotide bearing such bases, according to the so-called “wobble” rules (see, e.g., Murphy, FV IV & V Ramakrishnan, V., Nature Structural and Molecular Biology 11:1251-1252 (2004)). For example, a nucleotide comprising inosine as its base can base pair with nucleotides containing adenine, cytosine, or uracil. Thus, nucleotides containing uracil, guanine, or adenine can be replaced in the nucleotide sequences of an Inhibitory RNA described herein by a nucleotide containing, for example, inosine. It will be understood that the terms “complementary”, “perfectly complementary”, and “substantially complementary.” can be used with respect to the base matching between any two nucleic acids, e.g., the base matching between the sense strand and the antisense strand of a double stranded nucleic acid, or portion thereof. “Hybridize”, as used herein, refers to the interaction between two nucleic acid sequences (which in some embodiments may be part of the same nucleic acid molecule and in other embodiments may be or include part(s) of different nucleic acid molecules) comprising or consisting of complementary portions such that a duplex structure (i.e., an intramolecular or intermolecular duplex) is formed that is stable under the particular conditions of interest, as will be understood by the ordinary skilled artisan.

Comprising: The term “comprising” means that other elements can also be present in addition to the defined elements presented. The use of “comprising” indicates inclusion rather than limitation.

Consisting of: The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of additional elements that do not materially affect the basic and novel or functional characteristic(s) of that embodiment of the invention.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously: in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Domain: The term “domain” as used herein refers to a section or portion of an entity: In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide (e.g., the Ig3 domain of a MuSK protein): in some such embodiments, a domain is characterized by a particular structural element (e.g., a particular amino acid sequence or sequence motif, a-helix character, b-sheet character, coiled-coil character, random coil character, etc.), and/or by a particular functional feature (e.g., binding activity, enzymatic activity, folding activity, signaling activity, etc.).

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length: in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation): (3) transport of an RNA transcript (e.g., from nucleus to cytoplasm; and/or (4) translation of an RNA into a polypeptide or protein; and/or (4) post-translational modification of a polypeptide or protein.

Isolated or Partially Purified: The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen-binding portion thereof, or CAR described herein is purified.

Engineered: As used herein, “engineered” refers to the aspect of having been manipulated by the hand of man. For example, an antibody, antibody reagent, antigen-binding portion thereof, CAR or bispecific antibody is considered to be “engineered” when the sequence of the antibody, antibody reagent, antigen-binding portion thereof, CAR or bispecific antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene. In some embodiments, a gene product may be or comprise a particular processed form of an RNA transcript (e.g., a particular edited form, a particular splice form, a particular capped form, etc).

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively; be determined using the GAP program in the GCG software package using an NWSgapdna.CMP matrix.

Improve, “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual, a single cell, or cell population) under otherwise comparable conditions absent presence of (e.g., prior to and/or after) a particular agent or treatment, or in presence of an appropriate reference agent (e.g., a positive control agent or a negative control agent). In some embodiments, an appropriate reference measurement may be or comprise a measurement in comparable system known or expected to respond in a particular way, in presence of the relevant agent or treatment. Those skilled in the art will appreciate that an “improvement”, “increase”, “reduction”, etc typically refers to a statistically significant change. Moreover, those skilled in the art will understand from context what magnitude of change may be relevant. For example, in some embodiments, a change may be a “fold” change—i.e., so that a “changed” value represents a 1.1, 1.2, 2, 3, 4, 5, 6, 7, 8, 9, 10, 15, 20, 30, 40, 50 or more (e.g., 500, 1000 times) (including all integers and decimal points in between and above 1), e.g., 1.5, 1.6, 1.7. 1.8, etc.)-fold difference relative to the relevant reference. Alternatively or additionally, in some embodiments, a “change” may be a “percentage” change, so that a “changed” value represents a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase or decrease, including all integers and decimal points in between), relative to the relevant reference.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Internucleotidic linkage: As used herein, the phrase “internucleotidic linkage” refers generally to the phosphorus-containing linkage between nucleotide units of an oligonucleotide, and is interchangeable with “inter-sugar linkage” and “phosphorus atom bridge,” as used above and herein. In some embodiments, an internucleotidic linkage is a phosphodiester linkage, as found in naturally occurring DNA and RNA molecules. In some embodiments, an internucleotidic linkage is a “modified internucleotidic linkage” wherein each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described below. In some embodiments, an internucleotidic linkage is a phosphotriester linkage, phosphorothioate diester linkage

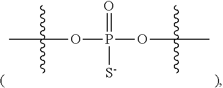

or modified phosphorothioate triester linkage. It is understood by a person of ordinary skill in the art that the internucleotidic linkage may exist as an anion or cation at a given pH due to the existence of acid or base moieties in the linkage. In some embodiments, an internucleotide linkage may be a chiral linkage.

Long-term Administration: As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

Moiety: Those skilled in the art will appreciate that a “moiety” is a defined chemical group or entity with a particular structure and/or or activity, as described herein.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside): in some embodiments, “nucleic acid” refers to an oligonucleotide chain comprising individual nucleic acid residues. In some embodiments, a “nucleic acid” is or comprises RNA: in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded: in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Oligonucleotide: As used herein, the term “oligonucleotide” refers to a polymer or oligomer of nucleotide monomers, containing any combination of nucleobases, modified nucleobases, sugars, modified sugars, phosphate bridges, or modified phosphorus atom bridges (also referred to herein as “internucleotidic linkage”, defined further herein). Oligonucleotides can be single-stranded or double-stranded. A single-stranded oligonucleotide can have double-stranded regions and a double-stranded oligonucleotide can have single-stranded regions. Example oligonucleotides include, but are not limited to structural genes, genes including control and termination regions, self-replicating systems such as viral or plasmid DNA, single-stranded and double-stranded siRNAs and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides. Double-stranded and single-stranded oligonucleotides that are effective in inducing RNA interference are also referred to as siRNA, RNAi agent, or iRNA agent, herein. In some embodiments, these RNA interference inducing oligonucleotides associate with a cytoplasmic multi-protein complex known as RNAi-induced silencing complex (RISC). In many embodiments, single-stranded and double-stranded RNAi agents are sufficiently long that they can be cleaved by an endogenous molecule, e.g., by Dicer, to produce smaller oligonucleotides that can enter the RISC machinery and participate in RISC mediated cleavage of a target sequence, e.g. a target mRNA.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements (e.g., promoters, enhancers, etc.) are contiguous (e.g., covalently linked) with the coding elements of interest: in some embodiments, control elements act in trans- or cis- with the coding functional element of interest.

Patient: As used herein, the term “patient” refers to any organism to which a provided composition (e.g., an agonizing agent such as an ASO) is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is Alzheimer's disease or other disease characterized by neurodegeneration. In some embodiments, the disorder or condition is muscular dystrophy or other disease characterized by neuromuscular dysfunction. In some embodiments, the patient is receiving or has received certain therapy to diagnose and/or to treat a disease, disorder, or condition.

Pharmaceutical composition: As used herein, the term “pharmaceutical composition” refers to an active agent (e.g., MuSK-targeting oligonucleotide), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue: parenteral administration, for example, by subcutaneous, intramuscular, intravenous, intraperitoneal, intrathecal, intravenous, intraventricular or epidural injection as, for example, a sterile solution or suspension, or sustained-release formulation: topical application, for example, as a cream, ointment, or a controlled-release patch or spray applied to the skin, lungs, or oral cavity: intravaginally or intrarectally, for example, as a pessary, cream, or foam: sublingually: ocularly: transdermally: or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups. e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each internucleotidic linkage, e.g., phosphate group, independently exists in its salt form (e.g., if sodium salt, —O—P(O)(ONa)—O—). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group, if any, exists as a salt form (all sodium salt).

Polypeptide: As used herein, the term “polypeptide,” which is interchangeably used herein with the term “protein,” refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure: in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known: in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class: in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity: often greater than 90% or even 95%, 96%, 97%, 98%, or 99%. Such a conserved region usually encompasses at least 3-4 and often up to 20 or more amino acids: in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein. In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan. In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell: polypeptides isolated from a recombinant, combinatorial human polypeptide library: polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Small molecule: As used herein, the term “small molecule” means a low molecular weight organic and/or inorganic compound. In general, a “small molecule” is a molecule that is less than about 5 kilodaltons (KD) in size. In some embodiments, a small molecule is less than about 4 kD, 3 kD, about 2 kD, or about 1 kD. In some embodiments, the small molecule is less than about 800 daltons (D), about 600 D, about 500 D, about 400) D, about 300 D, about 200 D. or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide: for example, in some embodiments, a small molecule is not a glycoprotein, proteoglycan, glycolipid, etc.). In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers: in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for 12C; ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound): in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound: a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond: a preparation in which one or more atoms is a different isotope than is present in a reference preparation may be considered to be a different form: etc.

Specific binding: As used herein, the term “specific binding” refers to an ability to discriminate between possible binding partners in the environment in which binding is to occur. A binding agent that interacts with one particular target when other potential targets are present is said to “bind specifically” to the target (e.g., a target amino acid or nucleic acid sequence on a target protein/gene of interest) with which it interacts. In some embodiments, specific binding is assessed by detecting or determining degree of association between the binding agent and its partner: in some embodiments, specific binding is assessed by detecting or determining degree of dissociation of a binding agent-partner complex: in some embodiments, specific binding is assessed by detecting or determining ability of the binding agent to compete an alternative interaction between its partner and another entity. In some embodiments, specific binding is assessed by performing such detections or determinations across a range of concentrations.

Specificity: As is known in the art, “specificity” is a measure of the ability of a particular ligand to distinguish its binding partner from other potential binding partners.

Subject: As used herein, the term “subject” refers an organism, typically a mammal (e.g., a human, in some embodiments including prenatal human forms). In some embodiments, a subject is suffering from a relevant disease, disorder or condition (e.g., Alzheimer's disease (AD), muscular dystrophy or other disease characterized by neurodegeneration or neuromuscular dysfunction). In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215 (3): 403-410, 1990; Altschul et al., Methods in Enzymology: Altschul et al., Nucleic Acids Res. 25:3389-3402, 1997: Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition (e.g., muscular dystrophy or other disease characterized by neuromuscular dysfunction) has been diagnosed with and/or displays one or more symptoms of a disease, disorder, and/or condition.

Susceptible to: An individual who is “susceptible to” a disease, disorder, and/or condition (e.g., Alzheimer's disease (AD), muscular dystrophy or other disease characterized by neurodegeneration or neuromuscular dysfunction) is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition (e.g., Alzheimer's disease (AD), muscular dystrophy or other disease characterized by neurodegeneration or neuromuscular dysfunction) is reduced in magnitude (e.g., intensity, severity, etc.) and/or frequency. For purposes of clarity, a delay in the onset of a particular symptom is considered one form of reducing the frequency of that symptom.

Target gene: A “target gene”, as used herein, refers to a gene whose expression is to be modulated, e.g., through modifying splice activity (e.g., by inducing exon-skipping). As used herein, the term “target portion” or “target region” refers to a contiguous portion of the nucleotide sequence of a target gene. In some embodiments, a target portion or target region is one or more exons within the target gene sequence. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition (e.g., one or more symptoms or features of Alzheimer's disease (AD), muscular dystrophy or other disease characterized by neurodegeneration or neuromuscular dysfunction).

Therapeutically effective amount: As used herein, the term “therapeutically effective amount” means an amount of a substance (e.g., a therapeutic agent, composition, and/or formulation) that elicits a desired biological response when administered as part of a therapeutic dosing regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. As used herein, the terms “effective amount” and “therapeutically-effective amount” include an amount sufficient to prevent or ameliorate a manifestation of disease or medical condition, such as Alzheimer's disease (AD), Parkinson's disease, or another disease characterized by neurodegeneration, reduced mobility, metabolism, and quality of life resulting from muscle wasting in cancer patients, elderly patients, and many others with no history of neuromuscular dysfunction, in addition to muscular dystrophies such as Becker, Congenital, Distal, Duchenne, Emery-Dreifuss, Facioscapulohumeral, Limb-girdle, Myotonic. Oculopharyngeal Muscular Dystrophy: It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In some embodiments, a therapeutically effective amount is administered in a single dose: in some embodiments, multiple unit doses are required to deliver a therapeutically effective amount.

Treating: As used herein, the term “treating” refers to providing treatment. i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. Beneficial or desired clinical results include, but are not limited to, alleviation of one or more symptom(s), diminishment of extent of the deficit, stabilized (i.e., not worsening) state of a muscular dystrophy, delay or slowing of muscle wasting, and an increased lifespan as compared to that expected in the absence of treatment. Treating can include administering an agent to the subject following the development of one or more symptoms or manifestations indicative of Alzheimer's disease (AD), muscular dystrophy or other disease characterized by neurodegeneration or neuromuscular dysfunction, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed Alzheimer's disease, muscular dystrophy or other disease characterized by neurodegeneration, neuromuscular dysfunction or is at increased risk of developing such a disorder relative to a member of the general population. A composition of the disclosure can be administered prophylactically, i.e., before development of any symptom or manifestation of the condition. Typically in this case the subject will be at risk of developing the condition.

Variant: As used herein in the context of molecules, e.g., nucleic acids (e.g., ASOs), proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function: a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

### Neurogenesis

Neurogenesis occurs in distinct regions of the adult mammalian brain. Neural stem cells (NSCs) are the endogenous source of new neurons and are active throughout life in virtually all mammals, including humans (Eriksson et al., 1998: Ernst et al., 2014; Moreno-Jiménez et al., 2019; Spalding et al., 2013). Extensive work in rodent models shows that neurogenesis supports learning and memory, sensory functions, and mood regulation (Enwere et al., 2004: Gage, 2019; Imayoshi et al., 2008: Zhang et al., 2008b). NSCs reside in two neurogenic niches: the subgranular zone (SGZ) in the dentate gyrus of the hippocampus and the subventricular zone (SVZ) lining the lateral ventricles. NSCs in the SVZ generate astrocytes and oligodendrocytes that support the existing circuitry as well as neurons in the olfactory bulb that are critical for olfactory discrimination. NSCs in the dentate gyrus give rise to granule neurons important for learning and memory. The majority of NSCs in the human brain are located in the hippocampus. Most hippocampal NSCs reside in a state of dormancy, termed quiescence. For neurogenesis to occur, quiescent NSCs must become activated in response to extrinsic or intrinsic cues. Newly born neurons functionally integrate into the local circuitry within the hippocampus and contribute to cognitive functions. The capacity of quiescent NSCs to activate declines during healthy and pathological aging and this loss precedes the decline in cognition (Enwere et al. 2004; Giachino et al. 2014; Capilla-Gonzalez et al. 2014).

Recent work indicates that endogenous or exogenous NSCs may be a valuable source of new neurons for the millions of individuals suffering from cognitive decline or brain injury. Activation of endogenous NSCs through exercise, re-feeding, or young blood improves age-related cognitive impairments in mice (Brandhorst et al., 2015: van Praag et al., 2005: Villeda et al., 2011, 2014). Accumulation of negative signals that degrade the neurogenic niche may contribute to reduction in newborn neurons in aging and AD. However, it has been difficult to harness the neurogenic potential of NSCs due to the lack of a specific therapeutic target that has the ability to overcome inhibitory signals. The present disclosure appreciates that recent mechanistic studies suggest that BMP signaling may represent a promising pathway to target in the context of AD and other diseases characterized by neurodegeneration. BMPs negatively regulate activation of NSCs (Mira et al., 2010) and are upregulated in AD and APP transgenic mice (Crews et al., 2010). The present disclosure provides technologies to specifically modulate BMP signaling in the neurogenic niche.

### Adult Hippocampal Neurogenesis

Adult Hippocampal Neurogenesis (AHN) is critical for normal learning and memory: AHN is abundant in healthy aged humans but is reduced from the earliest stages of Alzheimer's Disease (AD). AHN occurs throughout life in humans and is dramatically reduced in AD (Moreno-Jimenez et al., 2019; Steiner et al., 2019). Work in animal models has underscored the role of AHN in improving cognition in the face of AD pathology. Thus, restoring AHN may be an attractive target for an AD therapy: Interventions that promote adult hippocampal neurogenesis could enhance cognitive function and combat neurodegeneration.

AHN is critical for learning and memory. Newborn dentate granule cells are hyperexcitable and exhibit robust synaptic plasticity. Thus, dysregulation of the quiescent state and/or a failure to integrate into the mature circuitry are thought to contribute to the age-associated decline in neurogenesis and cognitive performance in aging and dementia.

AHN in Humans. Although AHN has been established in rodents and other species for decades, the existence of this process in human has been controversial until quite recently. Reports using BrdU incorporation (Eriksson et al., 1998), 14C dating (Ernst et al., 2014: Spalding et al., 2013) and markers of immature neuron (Boldrini et al., 2018; Moreno-Jiménez et al., 2019; Tobin et al., 2019) have provided independent lines of support from multiple labs supporting human AHN. Mathematical modeling of radiocarbon birth dating data yielded estimates that in humans 35% of hippocampal neurons are replaced by newborn neurons during adulthood at a rate of 1.75% per year (Spalding et al., 2013). On the other hand, another recent report using markers for immature neurons failed to detect significant levels of AHN in adult humans (Sorrells et al., 2018). In depth comparison of these reports have revealed several methodological and sample differences that seem likely to explain the failure to detect adult neurogenesis in the Sorrells et al., paper (Kempermann et al., 2018: Lucassen et al., 2019).

AHN and Alzheimer's Disease. Alzheimer's Disease is a devastating disorder. It is progressive, fatal and has an enormous societal and economic cost. 5.8 million Americans are living with AD. By 2050 this number is projected to rise to 14M. In 2019, AD and other dementias cost the nation $290 billion. By 2050, these costs could rise as high as $1.1 trillion. There are no effective treatments. There have been several recent high profile drug trials. Almost all of these trails have been based upon the ‘amyloid hypothesis’. There is an enormous unmet need for innovative and effective therapies for AD. Alzheimer's Disease devastates the hippocampus, a brain region necessary for encoding memories. The hippocampus is one of the two sites of adult neurogenesis in the brain. A large number of animal studies have shown that these adult-born neurons are necessary for learning and memory. A recent crucial study provided convincing evidence for robust neurogenesis in the adult human brain. Importantly, the level of adult neurogenesis in AD brain is greatly diminished compared to age-matched controls. (See E. P. Moreno-Jimenez et al. Nature Med. https://doi. org/10.1038/s41591-019-0375-9; 2019; See also related Editorial in Nature 567:433; 28 Mar. 2019). Thus, promoting adult neurogenesis is emerging as a highly attractive target in treating AD.

The hippocampus is one of the earliest and most affected brain regions in AD and its atrophy is a hallmark of disease progression (Allison et al., 2019). Moreover, work in both rodents and humans has demonstrated that hippocampal-dependent learning is impaired in the Alzheimer's setting (Crews et al., 2010). Notably, AHN levels in AD patients are only 30% of those observed in age-matched controls (Moreno-Jiménez et al., 2019). Critically, a recent mouse study using genetically diverse AD mouse models showed that the total number of hippocampal neurons (NeuN+cells) correlates with cognition (Neuner Neuron 2019). Finally, a recent study has shown that exercise-mediated rescue of pathology in AD mice requires AHN, and that AHN ablation alone exacerbates cognitive defects in these mice (Choi et al., 2018). Thus, strategies to compensate for the degeneration of hippocampal neurons through enhancing endogenous neurogenesis have the potential to open a new pathway for treating Alzheimer's disease.

Other diseases associated with impaired AHN include e.g., diseases and disorders associated with progressive memory loss, such as Frontotemporal Dementia (Terreros-Roncal et al., 2019), stroke (Lindvall et al., 2015). Impaired AHN is also associated with psychiatric disorders such as major depressive disorder (MDD), bipolar disorder, schizophrenia, post-traumatic stress disorder (PTSD), substance-related and addictive disorders (Yun et al., 2016), and other diseases such as Temporal-Lobe Epilepsy (Cook et al., 1992), Hippocampal Sclerosis (Tai et al. 2018), Niemann Pick Type C (Hong et al., 2015), and Diabetes-mediated hippocampal neuronal loss (Ho et al, 2013: Gold et al., 2007).

### Subventricular Zone Neurogenesis

In addition to the hippocampus (i.e., the subgranular zone (SGZ) in the dentate gyrus of the hippocampus), NSCs reside in the subventricular zone (SVZ) lining the lateral ventricles. NSCs in the SVZ generate astrocytes and oligodendrocytes that support the existing circuitry as well as neurons in the olfactory bulb that are critical for olfactory discrimination. Recent evidence suggests that SVZ NSCs can give rise to terminally differentiated neurons in the striatum in response to ischemic stroke or neurodegenerative diseases (Arvidsson et al. 2002: Parent et al. 2002: Thored et al. 2006; Ernst et al. 2014).

The present disclosure recognizes that strategies to compensate for the degeneration of neurons in the SVZ through enhancing endogenous neurogenesis have the potential to open a new pathway for treating diseases specifically associated with striatal neurogenesis such as Parkinson's disease (which could benefit both from increasing AHN and striatal neurogenesis in the SVZ: Pitcher et al. 2012: Sterling et al. 2013) and Huntington's disease (Sassone et al., 2018). Other diseases including addiction (e.g., chronic cocaine use and lifelong cigarette smoking) are also associated with reduced striatal volume (Barros-Loscertales et al. 2011; Das et al., 2012) and have the potential to be treated through enhancing endogenous neurogenesis in the SVZ.

### MuSK and Neurogenesis

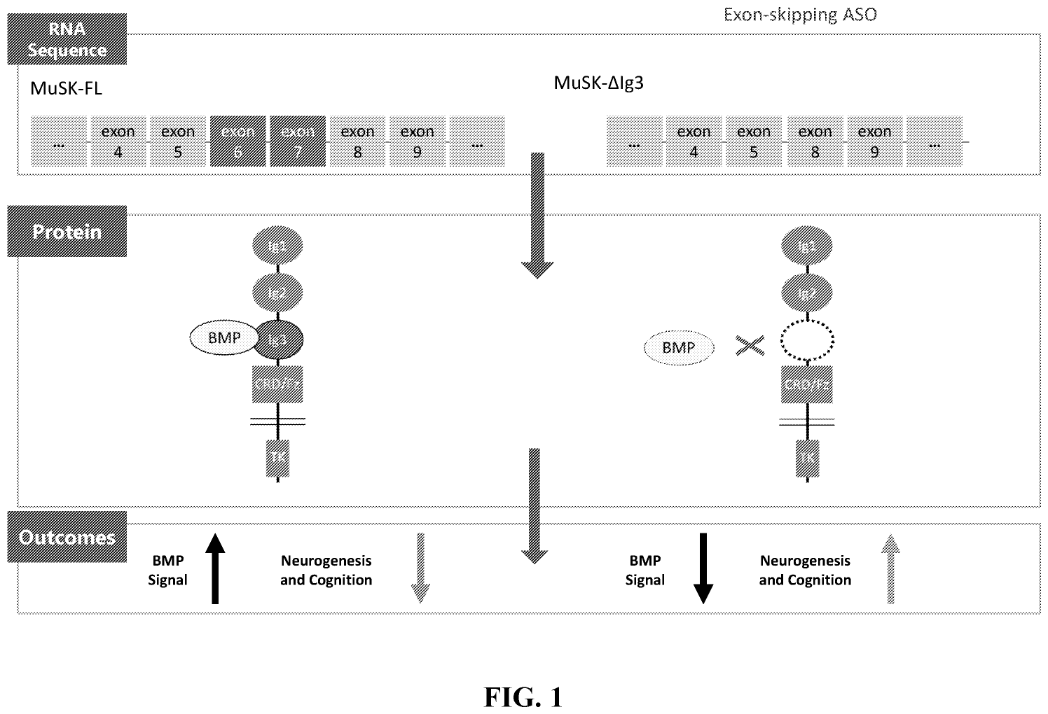

MuSK is a receptor tyrosine kinase comprised extracellularly of three Ig and one CRD/Fz domain and an intracellular tyrosine domain (TK: FIG. 1). The best understood function of MuSK is at the neuromuscular junction (NMJ) where agrin-LRP4 binding to the Ig1 domain triggers MuSK TK activity and synapse differentiation (Kim et al., 2008: Zhang et al., 2008a).

The MuSK-BMP Pathway. The brain harbors neural stem cells (NSCs) that generate neurons and glial cells throughout life (Moreno-Jiménez et al., 2019; Steiner et al., 2019). BMPs regulate at least two important NSC decision points: 1) quiescence, where proliferating stem cells exit the cell cycle and return to replenish a reserve pool that can supply fresh stem cells; and: 2) differentiation into mature progeny (Mira et al., 2010). The present disclosure contemplates that manipulating the BMP pathway in NSCs is an attractive target for regulating neurogenesis in the adult brain.

It was recently discovered that MuSK is also a BMP co-receptor that binds BMP and its receptors ALK3 and 6, upregulates BMP signaling and shapes the composition of the transcriptional response in myogenic cells (Yilmaz et al., 2016). This BMP signaling pathway neither regulates nor requires MuSK TK activity nor is it activated by agrin-LRP4. Importantly, the MuSK Ig3 domain is necessary for high affinity BMP binding but is dispensable for agrin-LRP4 TK activation. Moreover, the Ig3 domain is endogenously alternatively spliced, including in the brain (Garcia-Osta et al., 2006; Hesser et al., 1999). Since BMP signaling induces NSC quiescence and can inhibit integration of newborn neurons we have found that restraining BMP drive by reducing MuSK-BMP signaling could increase neurogenesis (FIG. 1).

Yilmaz et al. 2016 discloses that the ‘Ig3’ domain of MuSK is required for high affinity binding of BMPs. The major species of MuSK expressed endogenously is full length. This Ig3 domain can be alternatively spliced endogenously, creating an isoform termed “Δg3MuSK”. This splicing entails the coordinated removal of exons 6 and 7 from the MuSK pre-mRNA.

Exemplary amino acid sequences of human and mouse MuSK Ig3 domains (i.e., of MuSK Ig3 domain polypeptides) are as set out below:

MuSK HUMAN_Ig3_Domain:
(SEQ ID NO: 116)
ARILRAPESHNVTFGSFVTLHCTATGIPVPTITWIENGNAVSSGSIQES
VKDRVIDSRLQLFITKPGLYTCIATNKHGEKFSTAKAAATIS
MuSK MOUSE Ig3 Domain
(SEQ ID NO: 117)
ARILRAPESHNVTFGSFVTLRCTAIGIPVPTISWIENGNAVSSGSIQES
VKDRVIDSRLQLFITKPGLYTCIATNKHGEKFSTAKAAATVS

Among other things, the present disclosure provides compositions such as MuSK-targeting oligonucleotides that regulate MuSK alternative splicing as a strategy for increasing AHN in AD.

MuSK and Muscle Regeneration and/or Growth

Satellite cells account for about 5% of muscle nuclei and are distributed along mature, multinucleated myofibers, usually in a state of quiescence. When muscle is injured, satellite cells usually proliferate before either returning to quiescence or differentiating. Upon differentiation, satellite cells become committed myoblasts that fuse into myotubes, eventually forming mature myofibers in a process termed myogenesis. Bone morphogenetic protein (BMP) signaling regulates satellite cell dynamics and muscle regeneration both in vivo and in vitro by modulating transcriptional outputs. BMP signaling is not detectable in quiescent satellite cells, it is upregulated in proliferating satellite cells, and it is downregulated during differentiation. However, mediators regulating the balance between satellite cell proliferation and differentiation are unknown.

Muscle-Specific Kinase (MuSK), also known as Muscle-Associated Receptor Tyrosine Kinase, is a transmembrane protein that was first recognized for its essential role in the formation and maintenance of the neuromuscular junction (NMJ). MuSK has three extracellular Immunoglobulin (Ig)-like domains and a cysteine-rich frizzled (CRD/Fz) domain, as well as an intracellular tyrosine kinase (TK) domain. The Ig1, TK and potentially the CRD/Fz domains are required for NMJ formation and maintenance. The Ig1 and TK domains are essential for agrin-LRP4 signaling directing synaptic differentiation. For this reason, global deletion MuSK mice are neo-natal lethal. Two isoforms of MuSK that exist in vivo are: full-length (FL) MuSK and a naturally-occurring splice variant that lacks the Ig3 domain (ΔIg3-MuSK). FL MuSK mRNA levels are 10× higher than mRNA of ΔIg3-MuSK but the two are expressed coordinately.

Among other things, the present disclosure provides compositions such as MuSK-targeting oligonucleotides that regulate MuSK alternative splicing as a strategy for increasing muscle regeneration.

MuSK is activated by a nerve-derived proteoglycan called agrin. Agrin has been characterized for its role in the development of the neuromuscular junction during embryogenesis. Agrin is named based on its involvement in the aggregation of acetylcholine receptors during synaptogenesis. In humans, this protein is encoded by the AGRN gene. The agrin protein has nine domains homologous to protease inhibitors.

MuSK is expressed in muscle and is upregulated during muscle regeneration. Data suggest that MuSK is implicated in BMP signaling in myogenesis. MuSK can act as a BMP co-receptor that binds BMP2, BMP4, and BMP7 as well as the Type I BMP receptors ALK3 and ALK6. See, for example, Yilmaz et al., Sci. Signal. 9:ra87, doi: 10.1126/scisignal.aaf0890, 2016, incorporated herein by reference. The Ig3 domain of MuSK is required for high-affinity binding to BMP. MuSK upregulates BMP signaling as measured by BMP4-dependent phosphorylation of SMAD1/5/8. Importantly, MuSK-BMP signaling shapes the magnitude and composition of BMP-induced transcriptome in myoblasts and myotubes and this role is independent of any MuSK tyrosine kinase activity. MuSK is a BMP co-receptor that potentiates BMP signaling and regulates myogenic factors, such as myogenic factor 5 (Myf5), in immortalized myogenic cells.

Activated satellite cells express MuSK protein and disruption of MuSK-BMP signaling alters satellite cell proliferation in regenerating muscle in vivo. Additionally, previous studies have suggested the role of the MuSK-BMP pathway in satellite cells and muscle regeneration and that targeting MuSK-BMP pathway enhances muscle growth (see e.g., PCT Publication No. WO 2021/076883, which is incorporated by reference herein).

### MuSK-Targeting Oligonucleotides

As described herein, a strategy for regulating the MuSK-BMP pathway includes MuSK-targeting oligonucleotides (e.g., MuSK Ig3 targeting oligonucleotides). Specifically, the disclosure provides, among other things, oligonucleotides and compositions thereof that target regions spanning exon 6 and/or exon 7 of MuSK to induce exon-skipping of exon 6 and/or exon 7. Such alternative splicing activity leads to an increased expression of ΔIg3-MuSK.

The present disclosure also describes regions within the MuSK transcript at or near exon 6 and/or exon 7 that are particularly useful as target sequences for oligonucleotides in inducing exon skipping of exon 6 and/or exon 7, thereby generating and ΔIg3-MuSK transcripts.

In some embodiments, the present disclosure also provides specific oligonucleotides and combinations thereof that induce alternative splicing activity of MuSK and generate ΔIg3-MuSK transcripts.

In addition, the present disclosure provides compositions comprising one or more MuSK-targeting oligonucleotides that induce exon skipping of MuSK exons 6 and/or 7, and can be administered to a subject in a therapeutically effective amount to increase neurogenesis and/or muscle regeneration in the subject.

The disclosure includes compositions and methods related to one or more nucleotide sequences that are, comprise, or encode an oligonucleotide that binds to and inhibits expression of messenger RNA (mRNA) produced by a target gene (e.g., MuSK). Oligonucleotides can be single stranded (e.g., an antisense oligonucleotide) or double stranded nucleic acid. In some embodiments, an oligonucleotide comprises a double stranded RNA duplex such as microRNA (miRNA) or small interfering RNA (siRNA). In some embodiments, an oligonucleotide is an siRNA or miRNA, or a vector comprising a nucleotide sequence encoding an siRNA or miRNA. In some embodiments, an oligonucleotide is an antisense oligonucleotide (ASO), or a vector comprising a nucleotide sequence encoding an ASO.

In some embodiments, an oligonucleotide is capable of inhibiting expression of the full-length MuSK sequence comprises three extracellular Immunoglobulin (Ig)-like domains (Ig1, Ig2, and Ig3), a cysteine-rich frizzled (CRD/Fz) domain, as well as an intracellular tyrosine kinase (TK) domain.

As described herein, the oligonucleotides may target the MuSK sequence of human MuSK or one or more non-human species, e.g., a non-human primate MuSK, e.g., Macaca fascicularis MuSK (Gene ID 102127677), or e.g., chlorocebus sabaeus (Gene ID: 103219025), or murine MuSK (Gene ID: 18198). In some embodiments, a MuSK-targeting oligonucleotide comprises an antisense strand that is complementary to a target portion that is identical in the human and/or murine MuSK transcripts. In some embodiments, an oligonucleotide comprises a sequence that is complementary to a target portion of a human MuSK transcript that differs by 1, 2, or 3 nucleotides from a sequence in a murine or human MuSK transcript. It will be appreciated that an oligonucleotide that alters splicing of human MuSK may also alter splicing (i.e., induce exon skipping) of non-primate MuSK, e.g., rat or mouse MuSK, particularly if conserved regions of MuSK transcript are targeted.

The amino acid and nucleotide sequences of human MuSK are known in the art and can be found in publicly available databases, for example, the National Center for Biotechnology Information (NCBI) Reference Sequence (RefSeq) database, where the genomic nucleotide sequence is listed under RefSeq accession numbers NG_016016.2 (SEQ ID NO: 77) and forms of the mRNA/protein sequences are listed under NM_005592.4/NP_005583.1 (muscle, skeletal receptor tyrosine-protein kinase isoform 1), NM_001166280.2/NP_001159752.1 (muscle, skeletal receptor tyrosine-protein kinase isoform 2), NM_001166281.2/NP_001159753.1 (muscle, skeletal receptor tyrosine-protein kinase isoform 3) and NM_001369398.1/NP_001356327.1 (muscle, skeletal receptor tyrosine-protein kinase isoform 4). See www.ncbi.nlm.nih.gov/gene?Db-gene& Cmd=Details Search&Term=4593, which is incorporated herein by reference.

In some embodiments, an oligonucleotide comprises a nucleic acid strand that is complementary to a target portion of a MuSK transcript, e.g., MuSK mRNA (e.g., complementary to a nucleotide sequence that is at least 90%, 91%, 92%, 93%, 94%, 95%, 96%, 97%, 98%, 99%, or 100% identical to a target portion of SEQ ID NO: 77, e.g., Bld25/hu7-10 (SEQ ID NO: 63), Bld26/hu73 (SEQ ID NO: 64), etc.). In some embodiments, the target portion comprises a region that corresponds to positions 83776-83800 and/or 83854-83878 of SEQ ID No: 77 or a corresponding region of a different version of the genomic sequence MuSK. The target portion may be 15-30 nucleotides long, e.g., 15, 16, 17, 18, 19, 20, 21, 22, 23, 24, 25, 26, 27, 28, 29, or 30 nucleotides long, although shorter and longer target portions are also contemplated.

In some embodiments, a target portion of a MuSK transcript, e.g., MuSK mRNA, comprises a sequence of ACTCTGTCAGGTTTCTTCTGGGTCCATTCAAGAGAGTGTGAAAGACCGAGTGATT GACTCAAGAC (region 1, SEQ ID: 126), or a region within or a portion thereof. In some embodiments, a target portion comprises a sequence that is at least 10%, 15%, 20%, 25%, 30%, 35%, 40%, 45%, or 50% identical to region 1, SEQ ID: 126. In some embodiments, a target portion comprises a sequence that is identical to at least 10 consecutive bases of region 1, SEQ ID: 126. In some embodiments, a target portion comprises a sequence that is identical to at least 15 consecutive bases of region 1, SEQ ID: 126. In some embodiments, a target portion comprises a sequence that is identical to at least 18 consecutive bases of region 1, SEQ ID: 126. In some embodiments, a target portion comprises a sequence that is identical to at least 19 consecutive bases of region 1, SEQ ID: 126. In some embodiments, a target portion comprises a sequence that is identical to at least 20 consecutive bases of region 1, SEQ ID: 126. In some embodiments, a target portion comprises a sequence that is identical to at least 21 consecutive bases of region 1, SEQ ID: 126. In some embodiments, a target portion comprises a sequence that is identical to at least 22 consecutive bases of region 1, SEQ ID: 126. In some embodiments, a target portion comprises a sequence that is identical to at least 23 consecutive bases of region 1, SEQ ID: 126. In some embodiments, a target portion comprises a sequence that is identical to at least 24 consecutive bases of region 1, SEQ ID: 126. In some embodiments, a target portion comprises a sequence that is identical to at least 25 consecutive bases of region 1, SEQ ID: 126. In some embodiments, a target portion comprises a sequence that is identical to at least 30 consecutive bases of region 1, SEQ ID: 126. In some embodiments, a target portion comprises a sequence that is identical to at least 35 consecutive bases of region 1, SEQ ID: 126. In some embodiments, a target portion comprises a sequence that is identical to at least 15 consecutive bases and no more than 30 consecutive bases of region 1, SEQ ID: 126.

In some embodiments, a target portion comprises a sequence that is identical to 10 consecutive bases of region 1, SEQ ID: 126. In some embodiments, a target portion comprises a sequence that is identical to 11 consecutive bases of region 1, SEQ ID: 126. In some embodiments, a target portion comprises a sequence that is identical to 12 consecutive bases of region 1, SEQ ID: 126. In some embodiments, a target portion comprises a sequence that is identical to 13 consecutive bases of region 1, SEQ ID: 126. In some embodiments, a target portion comprises a sequence that is identical to 14 consecutive bases of region 1, SEQ ID: 126. In some embodiments, a target portion comprises a sequence that is identical to 15 consecutive bases of region 1, SEQ ID: 126. In some embodiments, a target portion comprises a sequence that is identical to 16 consecutive bases of region 1, SEQ ID: 126. In some embodiments, a target portion comprises a sequence that is identical to 17 consecutive bases of region 1, SEQ ID: 126. In some embodiments, a target portion comprises a sequence that is identical to 18 consecutive bases of region 1, SEQ ID: 126. In some embodiments, a target portion comprises a sequence that is identical to 19 consecutive bases of region 1, SEQ ID: 126. In some embodiments, a target portion comprises a sequence that is identical to 20 consecutive bases of region 1, SEQ ID: 126. In some embodiments, a target portion comprises a sequence that is identical to 21 consecutive bases of region 1, SEQ ID: 126. In some embodiments, a target portion comprises a sequence that is identical to 22 consecutive bases of region 1, SEQ ID: 126. In some embodiments, a target portion comprises a sequence that is identical to 23 consecutive bases of region 1, SEQ ID: 126. In some embodiments, a target portion comprises a sequence that is identical to 24 consecutive bases of region 1, SEQ ID: 126. In some embodiments, a target portion comprises a sequence that is identical to 25 consecutive bases of region 1, SEQ ID: 126. In some embodiments, a target portion comprises a sequence that is identical to 26 consecutive bases of region 1, SEQ ID: 126. In some embodiments, a target portion comprises a sequence that is identical to 27 consecutive bases of region 1, SEQ ID: 126. In some embodiments, a target portion comprises a sequence that is identical to 28 consecutive bases of region 1, SEQ ID: 126. In some embodiments, a target portion comprises a sequence that is identical to 29 consecutive bases of region 1, SEQ ID: 126. In some embodiments, a target portion comprises a sequence that is identical to 30 consecutive bases of region 1, SEQ ID: 126.

In some embodiments, a target portion of a MuSK transcript, e.g., MuSK mRNA, comprises a sequence of GGGGAGAAGTTCAGTACTGCCAAGGCTGCAGCCACCATCAGCATAGCAGGTAGG ATGCCCCTTCACATTTG (region 2, SEQ ID 211), or a region within or a portion thereof. In some embodiments, a target portion comprises a sequence that is at least 10%, 15%, 20%, 25%, 30%, 35%, 40%, 45%, or 50% identical to region 2, SEQ ID: 211. In some embodiments, a target portion comprises a sequence that is identical to at least 10 consecutive bases of region 2, SEQ ID: 211. In some embodiments, a target portion comprises a sequence that is identical to at least 15 consecutive bases of region 2, SEQ ID: 211. In some embodiments, a target portion comprises a sequence that is identical to at least 18 consecutive bases of region 2, SEQ ID: 211. In some embodiments, a target portion comprises a sequence that is identical to at least 19 consecutive bases of region 2, SEQ ID: 211. In some embodiments, a target portion comprises a sequence that is identical to at least 20 consecutive bases of region 2, SEQ ID: 211. In some embodiments, a target portion comprises a sequence that is identical to at least 21 consecutive bases of region 2, SEQ ID: 211. In some embodiments, a target portion comprises a sequence that is identical to at least 22 consecutive bases of region 2, SEQ ID: 211. In some embodiments, a target portion comprises a sequence that is identical to at least 23 consecutive bases of region 2, SEQ ID: 211. In some embodiments, a target portion comprises a sequence that is identical to at least 24 consecutive bases of region 2, SEQ ID: 211. In some embodiments, a target portion comprises a sequence that is identical to at least 25 consecutive bases of region 2, SEQ ID: 211. In some embodiments, a target portion comprises a sequence that is identical to at least 30 consecutive bases of region 2, SEQ ID: 211. In some embodiments, a target portion comprises a sequence that is identical to at least 35 consecutive bases of region 2, SEQ ID: 211.

In some embodiments, a target portion comprises a sequence that is identical to 10 consecutive bases of region 2, SEQ ID: 211. In some embodiments, a target portion comprises a sequence that is identical to 11 consecutive bases of region 2, SEQ ID: 211. In some embodiments, a target portion comprises a sequence that is identical to 12 consecutive bases of region 2, SEQ ID: 211. In some embodiments, a target portion comprises a sequence that is identical to 13 consecutive bases of region 2, SEQ ID: 211. In some embodiments, a target portion comprises a sequence that is identical to 14 consecutive bases of region 2, SEQ ID: 211. In some embodiments, a target portion comprises a sequence that is identical to 15 consecutive bases of region 2, SEQ ID: 211. In some embodiments, a target portion comprises a sequence that is identical to 16 consecutive bases of region 2, SEQ ID: 211. In some embodiments, a target portion comprises a sequence that is identical to 17 consecutive bases of region 2, SEQ ID: 211. In some embodiments, a target portion comprises a sequence that is identical to 18 consecutive bases of region 2, SEQ ID: 211. In some embodiments, a target portion comprises a sequence that is identical to 19 consecutive bases of region 2, SEQ ID: 211. In some embodiments, a target portion comprises a sequence that is identical to 20 consecutive bases of region 2, SEQ ID: 211. In some embodiments, a target portion comprises a sequence that is identical to 21 consecutive bases of region 2, SEQ ID: 211. In some embodiments, a target portion comprises a sequence that is identical to 22 consecutive bases of region 2, SEQ ID: 211. In some embodiments, a target portion comprises a sequence that is identical to 23 consecutive bases of region 2, SEQ ID: 211. In some embodiments, a target portion comprises a sequence that is identical to 24 consecutive bases of region 2, SEQ ID: 211. In some embodiments, a target portion comprises a sequence that is identical to 25 consecutive bases of region 2, SEQ ID: 211. In some embodiments, a target portion comprises a sequence that is identical to 26 consecutive bases of region 2, SEQ ID: 211. In some embodiments, a target portion comprises a sequence that is identical to 27 consecutive bases of region 2, SEQ ID: 211. In some embodiments, a target portion comprises a sequence that is identical to 28 consecutive bases of region 2, SEQ ID: 211. In some embodiments, a target portion comprises a sequence that is identical to 29 consecutive bases of region 2, SEQ ID: 211. In some embodiments, a target portion comprises a sequence that is identical to 30 consecutive bases of region 2, SEQ ID: 211.

In some embodiments, the target portion comprises a sequence that is at least 90%, 91%, 92%, 93%, 94%, 95%, 96%, 97%, 98%, 99%, or 100% identical to any one of the sequences listed below in Table 1.

TABLE 1

| Region | SEQ | |
|---|---|---|
| Description | ID | 5′ to 3′ Sequence |
| Exon 6 | 39 | TGCATTTCTAAAAGACCACCCTAGC |
| Exon 6 | 40 | TCTAAAAGACCACCCTAGCTTGACC |
| Exon 6 | 41 | ACCCTAGCTTGACCATTTCCTGCAG |
| Exon 6 | 42 | CTGCAGGAGCGTCACTCACCACTTC |
| Exon 6 | 43 | CACTCACCACTTCTGTCTTCCTAAC |
| Exon 6 | 44 | TCTTCCTAACAGTTTTTGCCAGGAT |
| Exon 6 | 45 | GATCCTGCGGGCTCCTGAATCCCAC |
| Exon 6 | 46 | GGCTCCTGAATCCCACAATGTCACC |
| Exon 6 | 47 | ATCCCACAATGTCACCTTTGGCTCC |
| Exon 6 | 48 | TGTCACCTTTGGCTCCTTTGTGACC |
| Exon 6 | 49 | TGGCTCCTTTGTGACCCTGCACTGT |
| Exon 6 | 50 | GTGACCCTGCACTGTACAGCAACAG |
| Exon 6 | 51 | GTACAGCAACAGGCATTCCTGTCCC |
| Exon 6 | 52 | GCATTCCTGTCCCCACCATCACCTG |
| Exon 6 | 53 | CCACCATCACCTGGATTGAAAACGG |
| Exon 6 | 54 | GAAAACGGAAATGCTGTGAGTGTCA |
| Exon 6 | 55 | GTGAGTGTCATGTGTGTGGGGACTT |
| Exon 6 | 56 | TGTGGGGACTTGTCTGGGGAAGACC |
| Exon 7 | 57 | TTCCCTTCAAATCTACTGACATAGT |
| Exon 7 | 58 | AATCTACTGACATAGTATAGTGGGA |
| Exon 7 | 59 | AGTATAGTGGGAAATCCTTGACTGA |
| Exon 7 | 60 | GGGAAATCCTTGACTGAGTTCTTTT |
| Exon 7 | 61 | ACTGAGTTCTTTTATTTTCCTTTAC |
| Exon 7 | 62 | TTATTTTCCTTTACTCTGTCAGGTT |
| Exon 7 | 63 | ACTCTGTCAGGTTTCTTCTGGGTCC |
| Exon 7 | 64 | TCTTCTGGGTCCATTCAAGAGAGTG |
| Exon 7 | 65 | TCAAGAGAGTGTGAAAGACCGAGTG |
| Exon 7 | 66 | AAAGACCGAGTGATTGACTCAAGAC |
| Exon 7 | 67 | TGACTCAAGACTGCAGCTGTTTATC |
| Exon 7 | 68 | AGCTGTTTATCACCAAGCCAGGACT |
| Exon 7 | 69 | ATCACCAAGCCAGGACTCTACACAT |
| Exon 7 | 70 | CAGGACTCTACACATGCATAGCTAC |
| Exon 7 | 71 | ACACATGCATAGCTACCAATAAGCA |
| Exon 7 | 72 | GCTACCAATAAGCATGGGGAGAAGT |
| Exon 7 | 73 | GGGGAGAAGTTCAGTACTGCCAAGG |
| Exon 7 | 74 | CTGCAGCCACCATCAGCATAGCAGG |
| Exon 7 | 75 | CATCAGCATAGCAGGTAGGATGCCC |
| Exon 7 | 76 | AGGTAGGATGCCCCTTCACATTTGC |
| Exon 7 | 212 | TTTCTTCTGGGTCCATTCAA |
| Exon 7 | 213 | TTCTTCTGGGTCCATTCAAG |
| Exon 7 | 214 | TCTTCTGGGTCCATTCAAGA |
| Exon 7 | 215 | CTTCTGGGTCCATTCAAGAG |
| Exon 7 | 216 | TTCTGGGTCCATTCAAGAGA |
| Exon 7 | 217 | TCTGGGTCCATTCAAGAGAG |
| Exon 7 | 218 | CTGGGTCCATTCAAGAGAGT |
| Exon 7 | 219 | TGGGTCCATTCAAGAGAGTG |
| Exon 7 | 220 | GGGTCCATTCAAGAGAGTGT |
| Exon 7 | 221 | GGTCCATTCAAGAGAGTGTG |
| Exon 7 | 222 | GTCCATTCAAGAGAGTGTGA |
| Exon 7 | 223 | TCCATTCAAGAGAGTGTGAA |
| Exon 7 | 224 | CCATTCAAGAGAGTGTGAAA |
| Exon 7 | 225 | CATTCAAGAGAGTGTGAAAG |
| Exon 7 | 226 | ATTCAAGAGAGTGTGAAAGA |
| Exon 7 | 227 | TTCAAGAGAGTGTGAAAGAC |
| Exon 7 | 228 | CTTTACTCTGTCAGGTTTCTTCTGG |
| Exon 7 | 229 | TTACTCTGTCAGGTTTCTTCTGGGT |
| Exon 7 | 230 | TCTGTCAGGTTTCTTCTGGGTCCAT |
| Exon 7 | 231 | TGTCAGGTTTCTTCTGGGTCCATTC |
| Exon 7 | 232 | CAGGTTTCTTCTGGGTCCATTCAAG |
| Exon 7 | 233 | GGTTTCTTCTGGGTCCATTCAAGAG |
| Exon 7 | 234 | TTTCTTCTGGGTCCATTCAAGAGAG |
| Exon 7 | 235 | TTCTGGGTCCATTCAAGAGAGTGTG |
| Exon 7 | 236 | CTGGGTCCATTCAAGAGAGTGTGAA |
| Exon 7 | 237 | ACTCTGTCAGGTTTCTTCTGGGT |
| Exon 7 | 238 | ACTCTGTCAGGTTTCTTCTGG |
| Exon 7 | 239 | TCTGTCAGGTTTCTTCTGGGTCC |
| Exon 7 | 240 | TCTGTCAGGTTTCTTCTGGGT |
| Exon 7 | 241 | TGTCAGGTTTCTTCTGGGTCC |
| Exon 7 | 242 | TCAGGTTTCTTCTGGGTCCATTC |
| Exon 7 | 243 | TCAGGTTTCTTCTGGGTCCAT |
| Exon 7 | 244 | AGGTTTCTTCTGGGTCCATTCAA |
| Exon 7 | 245 | AGGTTTCTTCTGGGTCCATTC |
| Exon 7 | 246 | GTTTCTTCTGGGTCCATTCAA |
| Exon 7 | 247 | TTTCTTCTGGGTCCATTCAAGAG |
| Exon 7 | 248 | TTTCTTCTGGGTCCATTCAAG |
| Exon 7 | 249 | TCTTCTGGGTCCATTCAAGAGAG |
| Exon 7 | 250 | TTCTGGGTCCATTCAAGAGAG |
| Exon 7 | 251 | TCTTCTGGGTCCATTCAAGAG |
| Exon 7 | 252 | TTCTGGGTCCATTCAAGAGAGTG |
| Exon 7 | 253 | CTGGGTCCATTCAAGAGAGTG |

Administration of MuSK-targeting oligonucleotide(s) as described herein can reduce the level of full-length MuSK transcript or full-length MuSK protein in a subject or in a biological sample (e.g., a blood, serum or plasma sample, or a sample comprising hepatocytes) compared to a level before the administration of the composition. In some embodiments, the level of full-length MuSK transcript or full-length MuSK protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration.

In some embodiments, an oligonucleotide comprises a nucleic acid strand that is complementary to a target portion of a MuSK transcript, e.g., MuSK mRNA. In some embodiments, an oligonucleotide comprises a nucleic acid strand that is complementary to a nucleotide sequence that is at least 90%, 91%, 92%, 93%, 94%, 95%, 96%, 97%, 98%, 99%, or 100% identical to a target portion of a MuSK transcript, e.g., MuSK mRNA.

In some embodiments, an oligonucleotide comprises a sequence that is at least 90% identical to a portion of ACTCTGTCAGGTTTCTTCTGGGTCCATTCAAGAGAGTGTGAAAGACCGAGTGATT GACTCAAGAC (region 1, SEQ ID: 126) that includes at least 10, 15, 18, 19, 20, 21, 22, 23, 24, 25, 26, 27, 28, 29, or 30 consecutive bases of region 1, SEQ ID: 126. In some embodiments, an oligonucleotide comprises a sequence that is at least 95% identical to a portion of SEQ ID: 126 that includes at least 10, 15, 18, 19, 20, 21, 22, 23, 24, 25, 26, 27, 28, 29, or 30 consecutive bases of region 1, SEQ ID: 126. In some embodiments, an oligonucleotide comprises a sequence that is at least 90% identical to a portion of SEQ ID: 126 that includes at least 10 consecutive bases of region 1, SEQ ID: 126. In some embodiments, an oligonucleotide comprises a sequence that is at least 90% identical to a portion of SEQ ID: 126 that includes at least 15 consecutive bases of region 1, SEQ ID: 126. In some embodiments, an oligonucleotide comprises a sequence that is at least 90% identical to a portion of SEQ ID: 126 that includes at least 18 consecutive bases of region 1, SEQ ID: 126. In some embodiments, an oligonucleotide comprises a sequence that is at least 90% identical to a portion of SEQ ID: 126 that includes at least 19 consecutive bases of region 1, SEQ ID: 126. In some embodiments, an oligonucleotide comprises a sequence that is at least 90% identical to a portion of SEQ ID: 126 that includes at least 20 consecutive bases of region 1, SEQ ID: 126. In some embodiments, an oligonucleotide comprises a sequence that is at least 90% identical to a portion of SEQ ID: 126 that includes at least 21 consecutive bases of region 1, SEQ ID: 126. In some embodiments, an oligonucleotide comprises a sequence that is at least 90% identical to a portion of SEQ ID: 126 that includes at least 22 consecutive bases of region 1, SEQ ID: 126. In some embodiments, an oligonucleotide comprises a sequence that is at least 90% identical to a portion of SEQ ID: 126 that includes at least 23 consecutive bases of region 1, SEQ ID: 126. In some embodiments, an oligonucleotide comprises a sequence that is at least 90% identical to a portion of SEQ ID: 126 that includes at least 24 consecutive bases of region 1, SEQ ID: 126. In some embodiments, an oligonucleotide comprises a sequence that is at least 90% identical to a portion of SEQ ID: 126 that includes at least 25 consecutive bases of region 1, SEQ ID: 126. In some embodiments, an oligonucleotide comprises a sequence that is at least 90% identical to a portion of SEQ ID: 126 that includes at least 26 consecutive bases of region 1, SEQ ID: 126. In some embodiments, an oligonucleotide comprises a sequence that is at least 90% identical to a portion of SEQ ID: 126 that includes at least 27 consecutive bases of region 1, SEQ ID: 126. In some embodiments, an oligonucleotide comprises a sequence that is at least 90% identical to a portion of SEQ ID: 126 that includes at least 28 consecutive bases of region 1, SEQ ID: 126. In some embodiments, an oligonucleotide comprises a sequence that is at least 90% identical to a portion of SEQ ID: 126 that includes at least 29 consecutive bases of region 1, SEQ ID: 126. In some embodiments, an oligonucleotide comprises a sequence that is at least 90% identical to a portion of SEQ ID: 126 that includes at least 30 consecutive bases of region 1, SEQ ID: 126.

In some embodiments, an oligonucleotide comprises a sequence that is at least 90% identical to a portion of ACTCTGTCAGGTTTCTTCTGGGTCCATTCAAGAGAGTGTGAAAGACCGAGTGATT GACTCAAGAC (region 1, SEQ ID: 126) that includes 10, 15, 18, 19, 20, 21, 22, 23, 24, 25, 26, 27, 28, 29, or 30 consecutive bases of region 1, SEQ ID: 126. In some embodiments, an oligonucleotide comprises a sequence that is at least 95% identical to a portion of SEQ ID: 126 that includes 10, 15, 18, 19, 20, 21, 22, 23, 24, 25, 26, 27, 28, 29, or 30 consecutive bases of region 1. SEQ ID: 126. In some embodiments, an oligonucleotide comprises a sequence that is at least 90% identical to a portion of SEQ ID: 126 that includes 10 consecutive bases of region 1, SEQ ID: 126. In some embodiments, an oligonucleotide comprises a sequence that is at least 90% identical to a portion of SEQ ID: 126 that includes 15 consecutive bases of region 1, SEQ ID: 126. In some embodiments, an oligonucleotide comprises a sequence that is at least 90% identical to a portion of SEQ ID: 126 that includes 18 consecutive bases of region 1, SEQ ID: 126. In some embodiments, an oligonucleotide comprises a sequence that is at least 90% identical to a portion of SEQ ID: 126 that includes 19 consecutive bases of region 1, SEQ ID: 126. In some embodiments, an oligonucleotide comprises a sequence that is at least 90% identical to a portion of SEQ ID: 126 that includes 20 consecutive bases of region 1, SEQ ID: 126. In some embodiments, an oligonucleotide comprises a sequence that is at least 90% identical to a portion of SEQ ID: 126 that includes 21 consecutive bases of region 1, SEQ ID: 126. In some embodiments, an oligonucleotide comprises a sequence that is at least 90% identical to a portion of SEQ ID: 126 that includes 22 consecutive bases of region 1, SEQ ID: 126. In some embodiments, an oligonucleotide comprises a sequence that is at least 90% identical to a portion of SEQ ID: 126 that includes 23 consecutive bases of region 1, SEQ ID: 126. In some embodiments, an oligonucleotide comprises a sequence that is at least 90% identical to a portion of SEQ ID: 126 that includes 24 consecutive bases of region 1, SEQ ID: 126. In some embodiments, an oligonucleotide comprises a sequence that is at least 90% identical to a portion of SEQ ID: 126 that includes 25 consecutive bases of region 1, SEQ ID: 126. In some embodiments, an oligonucleotide comprises a sequence that is at least 90% identical to a portion of SEQ ID: 126 that includes 26 consecutive bases of region 1, SEQ ID: 126. In some embodiments, an oligonucleotide comprises a sequence that is at least 90% identical to a portion of SEQ ID: 126 that includes 27 consecutive bases of region 1, SEQ ID: 126. In some embodiments, an oligonucleotide comprises a sequence that is at least 90% identical to a portion of SEQ ID: 126 that includes 28 consecutive bases of region 1, SEQ ID: 126. In some embodiments, an oligonucleotide comprises a sequence that is at least 90% identical to a portion of SEQ ID: 126 that includes 29 consecutive bases of region 1, SEQ ID: 126. In some embodiments, an oligonucleotide comprises a sequence that is at least 90% identical to a portion of SEQ ID: 126 that includes 30 consecutive bases of region 1, SEQ ID: 126.

In some embodiments, an oligonucleotide comprises a nucleic acid strand that is complementary to a sequence of ACTCTGTCAGGTTTCTTCTGGGTCCATTCAAGAGAGTGTGAAAGACCGAGTGATT GACTCAAGAC (region 1, SEQ ID: 126). In some embodiments, an oligonucleotide comprises a nucleic acid strand that is complementary to a sequence of that is at least 10%, 15%, 20%, 25%, 30%, 35%, 40%, 45%, or 50% identical to region 1, SEQ ID: 126. In some embodiments, an oligonucleotide comprises a nucleic acid strand that is complementary to at least 10 consecutive bases of region 1, SEQ ID: 126. In some embodiments, an oligonucleotide comprises a nucleic acid strand that is complementary to at least 15 consecutive bases of region 1, SEQ ID: 126. In some embodiments, an oligonucleotide comprises a nucleic acid strand that is complementary to at least 18 consecutive bases of region 1, SEQ ID: 126. In some embodiments, an oligonucleotide comprises a nucleic acid strand that is complementary to at least 19 consecutive bases of region 1, SEQ ID: 126. In some embodiments, an oligonucleotide comprises a nucleic acid strand that is complementary to at least 20 consecutive bases of region 1, SEQ ID: 126. In some embodiments, an oligonucleotide comprises a nucleic acid strand that is complementary to at least 21 consecutive bases of region 1, SEQ ID: 126. In some embodiments, an oligonucleotide comprises a nucleic acid strand that is complementary to at least 22 consecutive bases of region 1, SEQ ID: 126. In some embodiments, an oligonucleotide comprises a nucleic acid strand that is complementary to at least 23 consecutive bases of region 1, SEQ ID: 126. In some embodiments, an oligonucleotide comprises a nucleic acid strand that is complementary to at least 24 consecutive bases of region 1, SEQ ID: 126. In some embodiments, an oligonucleotide comprises a nucleic acid strand that is complementary to at least 25 consecutive bases of region 1, SEQ ID: 126. In some embodiments, an oligonucleotide comprises a nucleic acid strand that is complementary to at least 26 consecutive bases of region 1, SEQ ID: 126. In some embodiments, an oligonucleotide comprises a nucleic acid strand that is complementary to at least 27 consecutive bases of region 1, SEQ ID: 126. In some embodiments, an oligonucleotide comprises a nucleic acid strand that is complementary to at least 28 consecutive bases of region 1, SEQ ID: 126. In some embodiments, an oligonucleotide comprises a nucleic acid strand that is complementary to at least 29 consecutive bases of region 1, SEQ ID: 126. In some embodiments, an oligonucleotide comprises a nucleic acid strand that is complementary to at least 30 consecutive bases of region 1, SEQ ID: 126. In some embodiments, an oligonucleotide comprises a nucleic acid strand that is complementary to at least 35 consecutive bases of region 1, SEQ ID: 126.

In some embodiments, an oligonucleotide comprises a nucleic acid strand that is complementary to 10 consecutive bases of region 1, SEQ ID: 126. In some embodiments, an oligonucleotide comprises a nucleic acid strand that is complementary to 11 consecutive bases of region 1, SEQ ID: 126. In some embodiments, an oligonucleotide comprises a nucleic acid strand that is complementary to 12 consecutive bases of region 1. SEQ ID: 126. In some embodiments, an oligonucleotide comprises a nucleic acid strand that is complementary to 13 consecutive bases of region 1, SEQ ID: 126. In some embodiments, an oligonucleotide comprises a nucleic acid strand that is complementary to 14 consecutive bases of region 1, SEQ ID: 126. In some embodiments, an oligonucleotide comprises a nucleic acid strand that is complementary to 15 consecutive bases of region 1, SEQ ID: 126. In some embodiments, an oligonucleotide comprises a nucleic acid strand that is complementary to 16 consecutive bases of region 1, SEQ ID: 126. In some embodiments, an oligonucleotide comprises a nucleic acid strand that is complementary to 17 consecutive bases of region 1, SEQ ID: 126. In some embodiments, an oligonucleotide comprises a nucleic acid strand that is complementary to 18 consecutive bases of region 1, SEQ ID: 126. In some embodiments, an oligonucleotide comprises a nucleic acid strand that is complementary to 19 consecutive bases of region 1, SEQ ID: 126. In some embodiments, an oligonucleotide comprises a nucleic acid strand that is complementary to 20 consecutive bases of region 1, SEQ ID: 126. In some embodiments, an oligonucleotide comprises a nucleic acid strand that is complementary to 21 consecutive bases of region 1. SEQ ID: 126. In some embodiments, an oligonucleotide comprises a nucleic acid strand that is complementary to 22 consecutive bases of region 1, SEQ ID: 126. In some embodiments, an oligonucleotide comprises a nucleic acid strand that is complementary to 23 consecutive bases of region 1, SEQ ID: 126. In some embodiments, an oligonucleotide comprises a nucleic acid strand that is complementary to 24 consecutive bases of region 1. SEQ ID: 126. In some embodiments, an oligonucleotide comprises a nucleic acid strand that is complementary to 25 consecutive bases of region 1, SEQ ID: 126. In some embodiments, an oligonucleotide comprises a nucleic acid strand that is complementary to 26 consecutive bases of region 1, SEQ ID: 126. In some embodiments, an oligonucleotide comprises a nucleic acid strand that is complementary to 27 consecutive bases of region 1, SEQ ID: 126. In some embodiments, an oligonucleotide comprises a nucleic acid strand that is complementary to 28 consecutive bases of region 1, SEQ ID: 126. In some embodiments, an oligonucleotide comprises a nucleic acid strand that is complementary to 29 consecutive bases of region 1, SEQ ID: 126. In some embodiments, an oligonucleotide comprises a nucleic acid strand that is complementary to 30 consecutive bases of region 1, SEQ ID: 126.

In some embodiments, an oligonucleotide comprises a sequence that is at least 90% identical to a portion sequence GGGGAGAAGTTCAGTACTGCCAAGGCTGCAGCCACCATCAGCATAGCAGGTAGG ATGCCCCTTCACATTTG (region 2, SEQ ID 211) that includes at least 10, 15, 18, 19, 20, 21, 22, 23, 24, 25, 26, 27, 28, 29, or 30 consecutive bases of region 2, SEQ ID: 211. In some embodiments, an oligonucleotide comprises a sequence that is at least 95% identical to a portion of SEQ ID: 211 that includes at least 10, 15, 18, 19, 20, 21, 22, 23, 24, 25, 26, 27, 28, 29, or 30 consecutive bases of region 2, SEQ ID: 211. In some embodiments, an oligonucleotide comprises a sequence that is at least 90% identical to a portion of SEQ ID: 211 that includes 10, 15, 18, 19, 20, 21, 22, 23, 24, 25, 26, 27, 28, 29, or 30 consecutive bases of region 2, SEQ ID: 211. In some embodiments, an oligonucleotide comprises a sequence that is at least 95% identical to a portion of SEQ ID: 211 that includes 10, 15, 18, 19, 20, 21, 22, 23, 24, 25, 26, 27, 28, 29, or 30 consecutive bases of region 2, SEQ ID: 211.

In some embodiments, an oligonucleotide comprises a nucleic acid strand that is complementary to a portion of sequence GGGGAGAAGTTCAGTACTGCCAAGGCTGCAGCCACCATCAGCATAGCAGGTAGG ATGCCCCTTCACATTTG (region 2, SEQ ID 211). In some embodiments, an oligonucleotide comprises a nucleic acid strand that is complementary to at least 10 consecutive bases of region 2, SEQ ID: 211. In some embodiments, an oligonucleotide comprises a nucleic acid strand that is complementary to at least 15 consecutive bases of region 2, SEQ ID: 211. In some embodiments, an oligonucleotide comprises a nucleic acid strand that is complementary to at least 18 consecutive bases of region 2, SEQ ID: 211. In some embodiments, an oligonucleotide comprises a nucleic acid strand that is complementary to at least 19 consecutive bases of region 2, SEQ ID: 211. In some embodiments, an oligonucleotide comprises a nucleic acid strand that is complementary to at least 20 consecutive bases of region 2, SEQ ID: 211. In some embodiments, an oligonucleotide comprises a nucleic acid strand that is complementary to at least 21 consecutive bases of region 2, SEQ ID: 211. In some embodiments, an oligonucleotide comprises a nucleic acid strand that is complementary to at least 22 consecutive bases of region 2, SEQ ID: 211. In some embodiments, an oligonucleotide comprises a nucleic acid strand that is complementary to at least 23 consecutive bases of region 2, SEQ ID: 211. In some embodiments, an oligonucleotide comprises a nucleic acid strand that is complementary to at least 24 consecutive bases of region 2, SEQ ID: 211. In some embodiments, an oligonucleotide comprises a nucleic acid strand that is complementary to at least 25 consecutive bases of region 2, SEQ ID: 211. In some embodiments, an oligonucleotide comprises a nucleic acid strand that is complementary to at least 30 consecutive bases of region 2, SEQ ID: 211. In some embodiments, an oligonucleotide comprises a nucleic acid strand that is complementary to at least 35 consecutive bases of region 2, SEQ ID: 211.

In some embodiments, an oligonucleotide comprises a nucleic acid strand that is complementary to 10 consecutive bases of region 2, SEQ ID: 211. In some embodiments, an oligonucleotide comprises a nucleic acid strand that is complementary to 11 consecutive bases of region 2, SEQ ID: 211. In some embodiments, an oligonucleotide comprises a nucleic acid strand that is complementary to 12 consecutive bases of region 2, SEQ ID: 211. In some embodiments, an oligonucleotide comprises a nucleic acid strand that is complementary to 13 consecutive bases of region 2, SEQ ID: 211. In some embodiments, an oligonucleotide comprises a nucleic acid strand that is complementary to 14 consecutive bases of region 2, SEQ ID: 211. In some embodiments, an oligonucleotide comprises a nucleic acid strand that is complementary to 15 consecutive bases of region 2, SEQ ID: 211. In some embodiments, an oligonucleotide comprises a nucleic acid strand that is complementary to 16 consecutive bases of region 2, SEQ ID: 211. In some embodiments, an oligonucleotide comprises a nucleic acid strand that is complementary to 17 consecutive bases of region 2, SEQ ID: 211. In some embodiments, an oligonucleotide comprises a nucleic acid strand that is complementary to 18 consecutive bases of region 2, SEQ ID: 211. In some embodiments, an oligonucleotide comprises a nucleic acid strand that is complementary to 19 consecutive bases of region 2, SEQ ID: 211. In some embodiments, an oligonucleotide comprises a nucleic acid strand that is complementary to 20 consecutive bases of region 2, SEQ ID: 211. In some embodiments, an oligonucleotide comprises a nucleic acid strand that is complementary to 21 consecutive bases of region 2, SEQ ID: 211. In some embodiments, an oligonucleotide comprises a nucleic acid strand that is complementary to 22 consecutive bases of region 2, SEQ ID: 211. In some embodiments, an oligonucleotide comprises a nucleic acid strand that is complementary to 23 consecutive bases of region 2, SEQ ID: 211. In some embodiments, an oligonucleotide comprises a nucleic acid strand that is complementary to 24 consecutive bases of region 2, SEQ ID: 211. In some embodiments, an oligonucleotide comprises a nucleic acid strand that is complementary to 25 consecutive bases of region 2, SEQ ID: 211. In some embodiments, an oligonucleotide comprises a nucleic acid strand that is complementary to 26 consecutive bases of region 2, SEQ ID: 211. In some embodiments, an oligonucleotide comprises a nucleic acid strand that is complementary to 27 consecutive bases of region 2, SEQ ID: 211. In some embodiments, an oligonucleotide comprises a nucleic acid strand that is complementary to 28 consecutive bases of region 2, SEQ ID: 211. In some embodiments, an oligonucleotide comprises a nucleic acid strand that is complementary to 29 consecutive bases of region 2, SEQ ID: 211. In some embodiments, an oligonucleotide comprises a nucleic acid strand that is complementary to 30 consecutive bases of region 2, SEQ ID: 211.

In some embodiments, MuSK-targeting oligonucleotides of the disclosure are antisense oligonucleotides comprising a nucleotide sequence that is at least 90%, 91%, 92%, 93%, 94%, 95%, 96%, 97%, 98%, 99%, or 100% identical to any one of SEQ ID NOs: 1-38, 127-142, 159-167, 177-193 in the following Table 2:

TABLE 2

| | MuSK | | |
|---|---|---|---|
| SEQ | Targeting | | |
| ID | Region ID | Oligo ID | 5′ to 3′ Sequence |
| 1 | hu6-75 | Bld1 | GCTAGGGTGGTCTTTTAGAAATGCA |
| 2 | hu6-69 | Bld2 | GGTCAAGCTAGGGTGGTCTTTTAGA |
| 3 | hu6-58 | Bld3 | CTGCAGGAAATGGTCAAGCTAGGGT |
| 4 | hu6-39 | Bld4 | GAAGTGGTGAGTGACGCTCCTGCAG |
| 5 | hu6-27 | Bld5 | GTTAGGAAGACAGAAGTGGTGAGTG |
| 6 | hu6-12 | Bld6 | ATCCTGGCAAAAACTGTTAGGAAGA |
| 7 | hu610 | Bld7 | GTGGGATTCAGGAGCCCGCAGGATC |
| 8 | hu619 | Bld8 | GGTGACATTGTGGGATTCAGGAGCC |
| 9 | hu628 | Bld9 | GGAGCCAAAGGTGACATTGTGGGAT |
| 10 | hu637 | Bld10 | GGTCACAAAGGAGCCAAAGGTGACA |
| 11 | hu646 | Bld11 | ACAGTGCAGGGTCACAAAGGAGCCA |
| 12 | hu656 | Bld12 | CTGTTGCTGTACAGTGCAGGGTCAC |
| 13 | hu669 | Bld13 | GGGACAGGAATGCCTGTTGCTGTAC |
| 14 | hu681 | Bld14 | CAGGTGATGGTGGGGACAGGAATGC |
| 15 | hu693 | Bld15 | CCGTTTTCAATCCAGGTGATGGTGG |
| 16 | hu6110 | Bld16 | TGACACTCACAGCATTTCCGTTTTC |
| 17 | hu6125 | Bld17 | AAGTCCCCACACACATGACACTCAC |
| 18 | hu6139 | Bld18 | GGTCTTCCCCAGACAAGTCCCCACA |
| 19 | hu7-75 | Bld19 | ACTATGTCAGTAGATTTGAAGGGAA |
| 20 | hu7-66 | Bld20 | TCCCACTATACTATGTCAGTAGATT |
| 21 | hu7-53 | Bld21 | TCAGTCAAGGATTTCCCACTATACT |
| 22 | hu7-45 | Bld22 | AAAAGAACTCAGTCAAGGATTTCCC |
| 23 | hu7-33 | Bld23 | GTAAAGGAAAATAAAAGAACTCAGT |
| 24 | hu7-22 | Bld24 | AACCTGACAGAGTAAAGGAAAATAA |
| 25 | hu7-10 | Bld25 | GGACCCAGAAGAAACCTGACAGAGT |
| 26 | hu73 | Bld26 | CACTCTCTTGAATGGACCCAGAAGA |
| 27 | hu717 | Bld27 | CACTCGGTCTTTCACACTCTCTTGA |
| 28 | hu730 | Bld28 | GTCTTGAGTCAATCACTCGGTCTTT |
| 29 | hu744 | Bld29 | GATAAACAGCTGCAGTCTTGAGTCA |
| 30 | hu758 | Bld30 | AGTCCTGGCTTGGTGATAAACAGCT |
| 31 | hu766 | Bld31 | ATGTGTAGAGTCCTGGCTTGGTGAT |
| 32 | hu776 | Bld32 | GTAGCTATGCATGTGTAGAGTCCTG |
| 33 | hu785 | Bld33 | TGCTTATTGGTAGCTATGCATGTGT |
| 34 | hu796 | Bld34 | ACTTCTCCCCATGCTTATTGGTAGC |
| 35 | hu7111 | Bld35 | CCTTGGCAGTACTGAACTTCTCCCC |
| 36 | hu7136 | Bld36 | CCTGCTATGCTGATGGTGGCTGCAG |
| 37 | hu7146 | Bld37 | GGGCATCCTACCTGCTATGCTGATG |
| 38 | hu7158 | Bld38 | GCAAATGTGAAGGGGCATCCTACCT |
| 127 | | Bld51 | TTGAATGGACCCAGAAGAAA |
| 128 | | Bld52 | CTTGAATGGACCCAGAAGAA |
| 129 | | Bld53 | TCTTGAATGGACCCAGAAGA |
| 130 | | Bld54 | CTCTTGAATGGACCCAGAAG |
| 131 | | Bld55 | TCTCTTGAATGGACCCAGAA |
| 132 | | Bld56 | CTCTCTTGAATGGACCCAGA |
| 133 | | Bld57 | ACTCTCTTGAATGGACCCAG |
| 134 | | Bld58 | CACTCTCTTGAATGGACCCA |
| 135 | | Bld59 | ACACTCTCTTGAATGGACCC |
| 136 | | Bld60 | CACACTCTCTTGAATGGACC |
| 137 | | Bld61 | TCACACTCTCTTGAATGGAC |
| 138 | | Bld62 | TTCACACTCTCTTGAATGGA |
| 139 | | Bld63 | TTTCACACTCTCTTGAATGG |
| 140 | | Bld64 | CTTTCACACTCTCTTGAATG |
| 141 | | Bld65 | TCTTTCACACTCTCTTGAAT |
| 142 | | Bld66 | GTCTTTCACACTCTCTTGAA |
| 159 | | Bld25-1 | CCAGAAGAAACCTGACAGAGTAAAG |
| 160 | | Bld25-2 | ACCCAGAAGAAACCTGACAGAGTAA |
| 161 | | Bld25-3 | ATGGACCCAGAAGAAACCTGACAGA |
| 162 | | Bld25-4 | GAATGGACCCAGAAGAAACCTGACA |
| 163 | | Bld25-5 | CTTGAATGGACCCAGAAGAAACCTG |
| 164 | | Bld26-1 | CTCTTGAATGGACCCAGAAGAAACC |
| 165 | | Bld26-2 | CTCTCTTGAATGGACCCAGAAGAAA |
| 166 | | Bld26-3 | CACACTCTCTTGAATGGACCCAGAA |
| 167 | | Bld26-4 | TTCACACTCTCTTGAATGGACCCAG |
| 177 | | Bld25-A | ACCCAGAAGAAACCTGACAGAGT |
| 178 | | Bld25-B | CCAGAAGAAACCTGACAGAGT |
| 179 | | Bld25-C | GGACCCAGAAGAAACCTGACAGA |
| 180 | | Bld25-D | ACCCAGAAGAAACCTGACAGA |
| 181 | | Bld25-E | GGACCCAGAAGAAACCTGACA |
| 182 | | Bld25-5-A | GAATGGACCCAGAAGAAACCTGA |
| 183 | | Bld25-5-B | ATGGACCCAGAAGAAACCTGA |
| 184 | | Bld25-5-C | TTGAATGGACCCAGAAGAAACCT |
| 185 | | Bld25-5-D | GAATGGACCCAGAAGAAACCT |
| 186 | | Bld25-5-E | TTGAATGGACCCAGAAGAAAC |
| 187 | | Bld26-2-A | CTCTTGAATGGACCCAGAAGAAA |
| 188 | | Bld26-2-B | CTTGAATGGACCCAGAAGAAA |
| 189 | | Bld26-2-C | CTCTCTTGAATGGACCCAGAAGA |
| 190 | | Bld26-2-D | CTCTCTTGAATGGACCCAGAA |
| 191 | | Bld26-B | CTCTTGAATGGACCCAGAAGA |
| 192 | | Bld26-C | CACTCTCTTGAATGGACCCAGAA |
| 193 | | Bld26-D | CACTCTCTTGAATGGACCCAG |

In some embodiments, an oligonucleotide has a sequence that differs from that explicitly set forth in Table 2, for example by substitution of one or more residues, or types of residues, with an alternative residue or residue type—e.g., an analog or otherwise corresponding residue type. For example, in some embodiments, one or more “T” residues (or all “T” residues) of a sequence presented in Table 2 is a “U” residue or analog thereof.

In some embodiments, an oligonucleotide comprises mismatch(es) with the target. The base pair may be ranked on the basis of their propensity to promote dissociation or melting (e.g., on the free energy of association or dissociation of a particular pairing, the simplest approach is to examine the pairs on an individual pair basis, though next neighbour or similar analysis can also be used). In terms of promoting dissociation: A:U is preferred over G:C:G:U is preferred over G:C; and I:C is preferred over G:C (I=inosine).

In some embodiments, an oligonucleotide can include one or more (e.g., 2, 3, 4, or 5) nucleotides on the 3′ and/or 5′ end that is not complementary to the target sequence.

### Chemical Structures of MuSK-Targeting Oligonucleotides

Synthetic oligonucleotides provide useful molecular tools in a wide variety of applications. For example, oligonucleotides are useful in therapeutic, diagnostic, research, and new nanomaterials applications. The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modification, e.g., base modifications, sugar modifications, backbone modifications, etc., which, among other things, render these molecules less susceptible to degradation and improve other properties of oligonucleotides.

Among other things, the present disclosure encompasses the recognition that structural elements of oligonucleotides, such as base sequence, chemical modifications (e.g., modifications of sugar, base, and/or internucleotidic linkages, and patterns thereof), and/or stereochemistry (e.g., stereochemistry of backbone chiral centers (chiral internucleotidic linkages), and/or patterns thereof), can have significant impact on properties, e.g., stability, splicing-altering capabilities, etc. In some embodiments, oligonucleotide properties can be adjusted by optimizing chemical modifications (modifications of base, sugar, and/or internucleotidic linkage) and/or stereochemistry (pattern of backbone chiral centers).

In some embodiments, the present disclosure demonstrates that oligonucleotide compositions comprising oligonucleotides with controlled structural elements, e.g., controlled chemical modification, provide unexpected properties, including but not limited to those described herein. In some embodiments, provided compositions comprising oligonucleotides having chemical modifications (e.g., base modifications, sugar modification, internucleotidic linkage modifications, etc.) have improved properties, such as improved splicing-altering capabilities, or improved protein binding profile, and/or improved delivery, etc. Particularly, in some embodiments, the present disclosure provides compositions and methods for altering splicing of transcripts (e.g., MuSK transcripts). In some embodiments, the present disclosure provides compositions and methods for improving splicing of transcripts. In some embodiments, altered transcript splicing by provided compositions and methods include production of products having desired and/or improved biological functions, and/or knockdown of undesired product by, e.g., modifying splicing products so that undesired biological functions can be suppressed or removed.

In some embodiments, a splicing product is mRNA. In some embodiments, alteration comprises skipping one or more exons. In some embodiments, splicing of a transcript is improved in that exon skipping increases levels of mRNA and proteins that have improved beneficial activities compared with absence of exon skipping.

In some embodiments, splicing of a transcript is improved in that exon skipping lowers levels of mRNA and proteins that have undesired activities compared with absence of exon skipping. In some embodiments, a target is knocked down through exon skipping which, by skipping one or more exons, causes premature stop codon and/or frameshift mutations.

In some embodiments, an oligonucleotide of the disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products).

Modified nucleobases also include expanded-size nucleobases in which one or more aryl rings, such as phenyl rings, have been added.

In some embodiments, modified nucleobases are of any one of the following structures, optionally substituted:

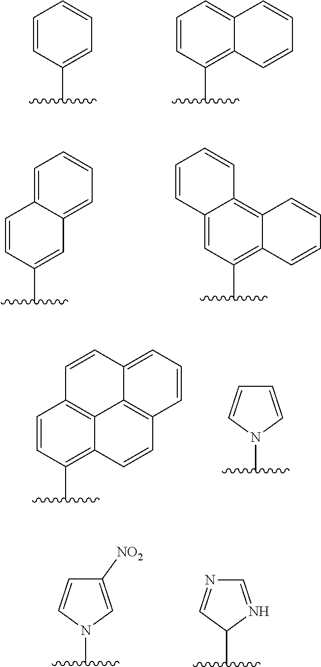

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, an oligonucleotide described herein includes nucleosides that incorporate modified nucleobases and/or nucleobases covalently bound to modified sugars. Some examples of nucleosides that incorporate modified nucleobases include 4-acetylcytidine; 5-(carboxyhydroxylmethyl) uridine; 2′-O-methylcytidine; 5-carboxymethylaminomethyl-2-thiouridine; 5-carboxy methylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-methylcytosine, 5-formylcytosine; 5-carboxylcytosine; N⁶-methyladenosine; 7-methylguanosine; 5-methylaminoethyluridine; 5-methoxyaminomethyl-2-thiouridine; beta, D-mannosylqueosine; 5-methoxy carbonylmethyluridine; 5-methoxyuridine; 2-methylthio-N⁶-isopentenyladenosine; N-((9-beta, D-ribofuranosyl-2-methylthiopurine-6-yl) carbamoyl) threonine; N-((9-beta,D-ribofuranosylpurine-6-yl)-N-methylcarbamoyl) threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or (S)-chirality at the 6′-position and include the analogs described in U.S. Pat. No. 7,399,845. In other embodiments, nucleosides include 5′-modified bicyclic nucleoside analogs that have either (R) or (S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

In some embodiments, an oligonucleotide described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context.

Other modified sugars can also be incorporated within an oligonucleotide molecule. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein: —O—(C₁-C₁₀ alkyl), —S(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH—(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), or salt thereof, wherein the alkyl, alkylene, alkenyl and alkynyl may be substituted or unsubstituted. Examples of substituents include, and are not limited to, —O(CH₂)ₙOCH₃, and —O(CH₂)ₙNH₂ or salt thereof, wherein n is from 1 to about 10, —OCH₂CH₂OMe (MOE) or salt thereof, —OCH₂CH₂N(CH₃)₂ (DMAOE) or salt thereof, —OCH₂CH₂OCH₂CH₂N(CH₃)₂ (DMAEOE) or salt thereof.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein: —O—(C₁-C₁₀ alkyl), —S(C₁-C₁₀ alkyl), —NH—(C₁-C₁₀ alkyl), or —N(C₁-C₁₀ alkyl)₂; —O—(C₂-C₁₀ alkenyl), —S(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C10 alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe (MOE).

Modified sugars also include locked nucleic acids (LNAs). In some embodiments, the locked nucleic acid has the structure indicated below. A locked nucleic acid of the structure below is indicated, wherein Ba represents a nucleobase or modified nucleobase as described herein, and wherein R^{2s} is —OCH₂C4′-

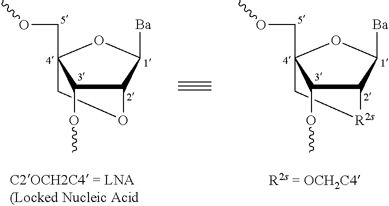

In some embodiments, each sugar of the oligonucleotide is or comprises a modified sugar moiety. In some embodiments, each sugar of the oligonucleotide is or comprises a 2′-MOE modified sugar. In some embodiments, each sugar of the oligonucleotide is or comprises a 2′-OMe modified sugar. In some embodiments, each sugar of the oligonucleotide is or comprises a 2′-OH modified sugar. In some embodiments, each sugar of the oligonucleotide is or comprises a 2′-H modified sugar.

In some embodiments, the present disclosure provides an oligonucleotide comprising 2′-MOE modified sugar, 2′-OMe modified sugar, 2′-OH modified sugar, 2′-H modified sugar, or combinations thereof. In some embodiments, a provided oligonucleotide comprises at least one 2′-MOE sugar and at least one 2′-OH sugar (RNA sugar). In some embodiments, a provided oligonucleotide comprises at least one 2′-MOE sugar and at least one 2′-H sugar (DNA sugar).

In some embodiments, the present invention provides an oligonucleotide comprising one or more modified internucleotidic linkages independently having the structure of formula I:

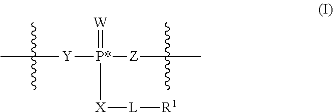

wherein:
P* is an asymmetric phosphorus atom and is either Rp or Sp configuration:
W is O, S or Se:
each of X, Y and Z is independently-O—, —S—, —N(-L-R¹)—, or L;

L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;

R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC (O)N(R′)—, —S(O)—, —S(O)₂—, —S(O)₂N(R′)—, —N(R′)S(O)₂—, —SC(O)—, —C(O)S—, —OC (O)—, or —C(O)O—;
each R′ is independently-R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring:
—Cy— is an optionally substituted bivalent ring selected from phenylene, carbocyclylene, arylene, heteroarylene, or heterocyclylene:
each R is independently hydrogen, or an optionally substituted group selected from C₁-C₆ aliphatic, phenyl, carbocyclyl, aryl, heteroaryl, or heterocyclyl; and
each

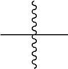

independently represents a connection to a nucleoside.

In some embodiments, the internucleotidic linkage having the structure of formula I is

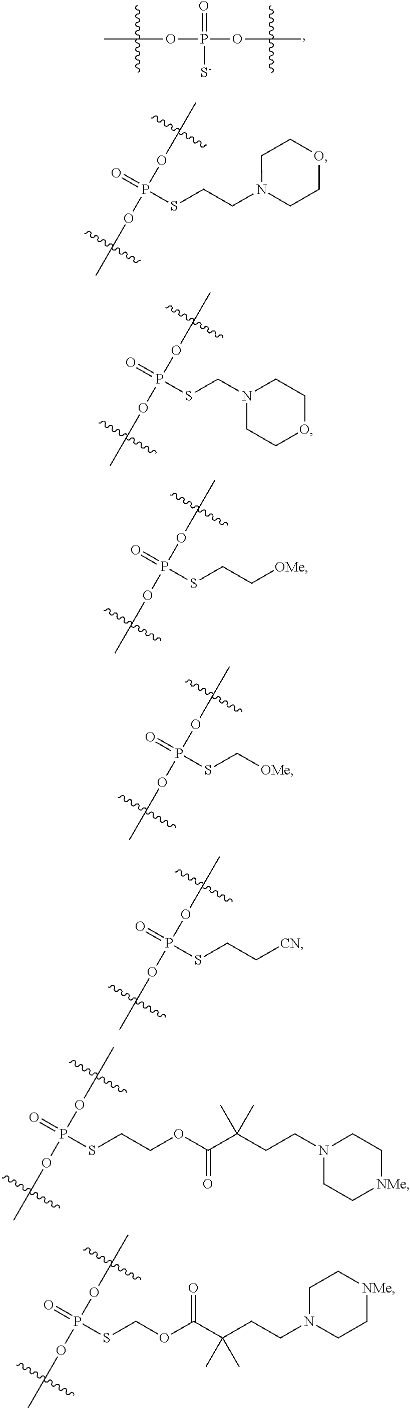

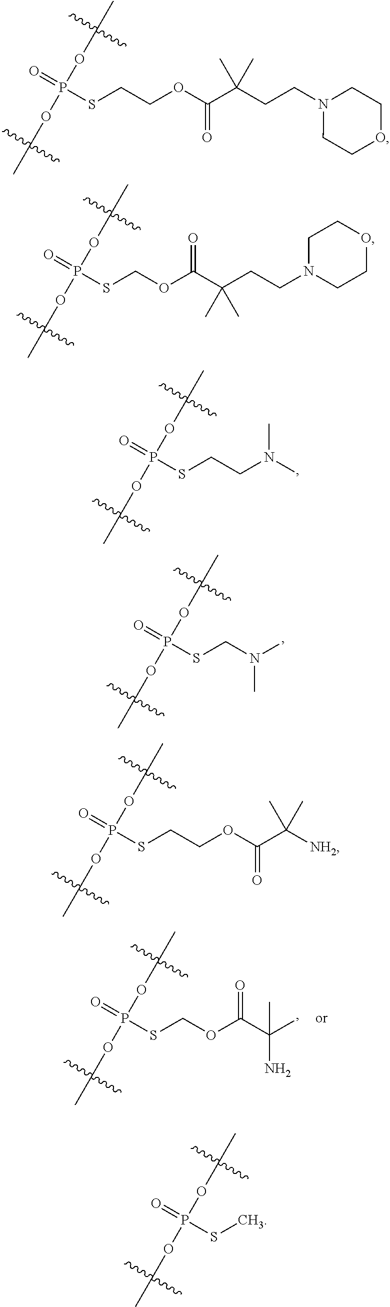

In some embodiments, an oligonucleotide comprises both non-natural internucleotidic linkage as described herein and natural phosphate linkage. In some embodiments, each internucleotidic linkages of the oligonucleotide is a non-natural internucleotidic linkage. In some embodiments, each internucleotidic linkages of the oligonucleotide is a chiral internucleotidic linkage. In some embodiments, each internucleotidic linkages of the oligonucleotide is a phosphorothioate linkage

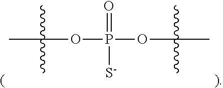

In some embodiments, each internucleotidic linkages of an oligonucleotide is a natural phosphate linkage. In some embodiments, an oligonucleotide comprises at least one natural phosphate linkage and at least one phosphodithioate linkage. In some embodiments, at least 50% internucleotidic linkages of an oligonucleotide are phosphodithioate linkages. In some embodiments, at least 60% internucleotidic linkages of an oligonucleotide are phosphodithioate linkages. In some embodiments, at least 70% internucleotidic linkages of an oligonucleotide are phosphodithioate linkages. In some embodiments, at least 80% internucleotidic linkages of an oligonucleotide are phosphodithioate linkages. In some embodiments, at least 90% internucleotidic linkages of an oligonucleotide are phosphodithioate linkages. In some embodiments, at least 94% internucleotidic linkages of an oligonucleotide are phosphodithioate linkages. In some embodiments, at least 95% internucleotidic linkages of an oligonucleotide are phosphodithioate linkages.

In some embodiments, at least 50% internucleotidic linkages of an oligonucleotide are natural phosphate linkages. In some embodiments, at least 60% internucleotidic linkages of an oligonucleotide are natural phosphate linkages. In some embodiments, at least 70% internucleotidic linkages of an oligonucleotide are natural phosphate linkages. In some embodiments, at least 80% internucleotidic linkages of an oligonucleotide are natural phosphate linkages. In some embodiments, at least 90% internucleotidic linkages of an oligonucleotide are natural phosphate linkages. In some embodiments, at least 94% internucleotidic linkages of an oligonucleotide are natural phosphate linkages. In some embodiments, at least 95% internucleotidic linkages of an oligonucleotide are natural phosphate linkages.

Among other things, the present disclosure provides oligonucleotides of various designs, which may comprises various nucleobases and patterns thereof, sugars and patterns thereof, internucleotidic linkages and patterns thereof, and/or additional chemical moieties and patterns thereof as described in the present disclosure. In some embodiments, provided oligonucleotides can downregulate the MuSK Ig3 domain protein expression, the MuSK Ig3 domain gene expression, and/or the MuSK Ig3 activation of BMP signaling level, thereby increasing adult hippocampal neurogenesis (AHN) and improving cognition in AD. In some embodiments, provided oligonucleotides can downregulate the MuSK Ig3 domain protein expression, the MuSK Ig3 domain gene expression, and/or the MuSK Ig3 activation of BMP signaling level, thereby increasing muscle regeneration. In some embodiments, provided oligonucleotides can direct a decrease in the expression, level and/or activity of MuSK Ig3 domain and/or one or more of its products in a cell of a subject or patient. In some embodiments, provided oligonucleotides can direct a decrease in the expression, level and/or activity of MuSK Ig3 domain and/or one or more of its products in a cell of a subject or patient, while the expression, level, and/or activity of all forms of MuSK remains substantially the same. In some embodiments, a cell normally expresses or produces protein encoded by MuSK Ig3 domain. In some embodiments, provided MuSK-targeting oligonucleotides can direct a decrease in the expression, level and/or activity of MuSK Ig3 domain gene or a gene product and have a base sequence which consists of, comprises, or comprises a portion (e.g., a span of 10, 11, 12, 13, 14, 15, 16, 17, 18, 19, 20 or more contiguous bases) of the base sequence of a oligonucleotide disclosed herein, wherein each T can be independently substituted with U and vice versa, and the oligonucleotide comprises at least one non-naturally-occurring modification of a base, sugar and/or internucleotidic linkage.

As described herein, the naturally highly abundant full length MuSK harbors the BMP-binding Ig3 domain and potentiates BMP signaling and thus restrains neurogenesis. In contrast, ΔIg3-MuSK has lower BMP signaling and promotes AHN and improves cognition. In some embodiments, the present disclosure provides exon-skipping MuSK-targeting oligonucleotides that switch MuSK from the AHN restraining full length MuSK to AHN permissive ΔIg3-MuSK splice form.

As described herein, the highly abundant full length MuSK harbors the BMP-binding Ig3 domain and potentiates BMP signaling and affects muscle regeneration. In contrast, ΔIg3-MuSK has lower BMP signaling and promotes muscle regeneration and/or prevents muscle fibrosis. In some embodiments, the present disclosure provides exon-skipping MuSK-targeting oligonucleotides that switch MuSK from the full length MuSK to the muscle-promoting ΔIg3-MuSK splice form.

In some embodiments, one or more skipped exons are selected from exon 6 and/or 7 of the MuSK gene. In some embodiments, exon 6 of MuSK is skipped. In some embodiments, exon 7 of MuSK is skipped. In some embodiments, both exons 6 and 7 of MuSK are skipped.

In some embodiments, a MuSK-targeting oligonucleotide described herein can provide exon-skipping of exon 6 and/or 7, but does not provide exon-skipping of exon 3 and/or 4.

In some embodiments, a MuSK-targeting oligonucleotide described herein can provide exon-skipping of exon 6 and/or 7 at a greater level than it provides exon-skipping of exon 3 and/or 4.

In some embodiments, a MuSK-targeting oligonucleotide described herein provides exon-skipping such that the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 40%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 40%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 70% and the level of MuSK transcripts including exons 3 and 4 or the level of MuSK protein forms including sequences encoded by exons 3 and 4, or both, decreases by less than 40%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 80% and the level of MuSK transcripts including exons 3 and 4 or the level of MuSK protein forms including sequences encoded by exons 3 and 4, or both, decreases by less than 40%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 90% and the level of MuSK transcripts including exons 3 and 4 or the level of MuSK protein forms including sequences encoded by exons 3 and 4, or both, decreases by less than 40%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 30%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 20%.

In some embodiments, a MuSK-targeting oligonucleotide alters the splicing of MuSK transcripts such that the level of MuSK transcripts including exons 6 and 7 or the level of MuSK protein forms including sequences encoded by exons 6 and 7, or both, decreases by greater than 60% and the level of MuSK transcripts including exons 3 and 4 or the level of MuSK protein forms including sequences encoded by exons 3 and 4, or both, decreases by less than 10%.

In various embodiments, an active compound is an oligonucleotide that directs skipping of one or more exons in a MuSK gene. In various embodiments, an active compound is an oligonucleotide that directs skipping of multiple exons in a MuSK gene. In some embodiments, an active compound is an oligonucleotide that directs skipping of exon 6, exon 7, or both in a MuSK gene. In some embodiments, an active compound is an oligonucleotide that directs skipping of exon 6 in a MuSK gene. In some embodiments, an active compound is an oligonucleotide that directs skipping of exon 7 in a MuSK gene. In some embodiments, an active compound is an oligonucleotide that directs skipping of exons 6 and 7 in a MuSK gene. In some embodiments, a plurality of oligonucleotides may be used together. In some such embodiments, two or more different exon skipping oligonucleotides (e.g., at least one that directs skipping of exon 6 and one that directs skipping of exon 7) may be used in combination. Alternatively or additionally, in some embodiments, at least one exon skipping oligonucleotide may be used in combination with at least one degrading oligonucleotide (e.g., that targets a transcript for RNase H degradation) which, for example, may target MuSK transcript(s) that include a functional Ig3 domain, or portion thereof.

In some embodiments, oligonucleotides are provided and/or utilized in salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H+) are of the same constitution and/or structure, such individual oligonucleotides may properly be considered to be of the same constitution and/or structure.

In some embodiments, an oligonucleotide composition comprises two or more oligonucleotides. In some embodiments, an oligonucleotide composition comprises two or more pluralities of oligonucleotides, wherein each plurality is independently a plurality of oligonucleotides as described herein. For example, in some embodiments, each plurality independently shares a same base sequence and the same internucleotidic linkages. In some embodiments, at least two pluralities or each plurality independently targets the same exon(s) of the same transcript (e.g., exons 6 and/or 7 of MuSK). In some embodiments, at least two pluralities or each plurality independently targets different exons of the same transcript (e.g., exons 3, 4, 6, and/or 7 of MuSK). In some embodiments, at least two pluralities or each plurality independently targets a different transcript of the same or different nucleic acids. In some embodiments, at least two pluralities or each plurality independently targets transcripts of a different gene. In some embodiments, at least two pluralities or each plurality independently targets different regions on the MuSK transcript. Among other things, such compositions may be utilized to target two or more targets, in some embodiments, simultaneously and in the same system.

### Characterization of MuSK-Targeting Oligonucleotides

MuSK-targeting oligonucleotides provided herein may be identified, assessed and/or characterized for one or more their physical/chemical properties and/or biological activities. Those skilled in the art will be aware of a variety of approaches, including particular assays, that may be utilized for such identification, assessment, and/or characterization.

In some embodiments, a MuSK-targeting oligonucleotide as described herein is characterized in that, for example, the MuSK-targeting oligonucleotide, when contacted with a cell expressing MuSK, will increase the level or activity of MuSK ΔIg3 mRNA and/or protein, e.g., relative to another MuSK form or other appropriate reference. In some embodiments, achieving such increase may be considered to represent “agonizing” MuSK ΔIg3.

In some embodiments, a MuSK-targeting oligonucleotide is characterized by its ability to alter splicing activity of MuSK pre-mRNA in a cell. For example, a cell may be transfected with a MuSK-targeting oligonucleotide, and after a period of incubation, expression of an alternative form of processed form of a MuSK RNA transcript (e.g., where exons 6 and 7 have been skipped), can be measured by RT-PCR. For example, the efficiency of MuSK exon skipping in cultured cells greater than 10%, 20%, 30%, 40%, 50%, 60%, 70%, 80%, 90%, 95%, or 95%.

In some aspects, a MuSK-targeting oligonucleotide increases of MuSK ΔIg3 mRNA. In some aspects, a MuSK-targeting oligonucleotide alters splicing of MuSK pre-mRNA. In some aspects, a MuSK-targeting oligonucleotide promotes the skipping of exon 6 and/or exon 7.

Modulation of expression of MuSK ΔIg3 can be measured in a bodily fluid of a subject treated with MuSK MR-targeting oligonucleotide, which may or may not contain cells: tissue: or organ of the animal. Methods of obtaining samples for analysis, such as body fluids (e.g., sputum, serum, CSF), tissues (e.g., biopsy), or organs, and methods of preparation of the samples to allow for analysis are well known to those skilled in the art. The effects of treatment on a subject can be assessed by measuring biomarkers associated with the target gene expression in one or more biological fluids, tissues or organs, collected from an animal contacted with one or more compositions described in this application.

In some embodiments, an increase in MuSK ΔIg3 mRNA means that the intracellular level of MuSK ΔIg3 mRNA is higher than a reference level, such as the level of MuSK ΔIg3 mRNA in a control (for example in a subject that is not being administered an MuSK-targeting oligonucleotide). An increase in intracellular MuSK ΔIg3 mRNA can be measured as an increase in the level of MuSK ΔIg3 protein and/or mRNA produced. In some embodiments, an increase in MuSK ΔIg3 mRNA can be determined by e.g., methods as described below in the examples, and/or by assay techniques such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect the presence of MuSK ΔIg3 mRNA or protein (e.g., in a subject or a sample obtained from a subject).

In some embodiments, by comparing the level of MuSK ΔIg3 mRNA in a sample obtained from a subject receiving a MuSK-targeting oligonucleotide treatment to a level of MuSK ΔIg3 mRNA in a subject not treated with a MuSK-targeting oligonucleotide, the extent to which the MuSK-targeting oligonucleotide treatment increased MuSK ΔIg3 mRNA can be determined. In some embodiments, the reference level of MuSK ΔIg3 mRNA is obtained from the same subject prior to receiving MuSK-targeting oligonucleotide treatment. In some embodiments, the reference level of MuSK ΔIg3 mRNA is a range determined by a population of subjects not receiving MuSK-targeting oligonucleotide treatment. In some embodiments, the level of full-length MuSK mRNA is compared to the level of MuSK ΔIg3 mRNA. In some embodiments, the ratio of the MuSK ΔIg3 mRNA to a full length MuSK mRNA (e.g., MuSK mRNA without exons 6 and 7) in a subject receiving a MuSK-targeting oligonucleotide treatment, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference ratio.

In some embodiments, an increased level of MuSK ΔIg3 mRNA is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference value.

In some embodiments, the increase of MuSK ΔIg3 mRNA in a subject can be indicated by the increase of MuSK ΔIg3 protein as compared to a reference level. In some embodiments, the reference level of MuSK ΔIg3 protein is the MuSK ΔIg3 protein level obtained from a subject having or at risk of having e.g., AD or a disease characterized by neurodegeneration. In some embodiments, the reference level of MuSK ΔIg3 protein is the MuSK ΔIg3 protein level obtained from a subject having or at risk of having e.g., neuromuscular dysfunction, a neurodegenerative disorder, a cardiac disorder (e.g., myocardial infarction, cardiomyopathy), or genetic diseases characterized by muscle wasting prior to treatment. Methods whereby bodily fluids, organs or tissues are contacted with an effective amount of one or more compositions described herein are also contemplated. Bodily fluids, organs or tissues can be contacted with one or more compositions comprising MuSK-targeting oligonucleotides, resulting in expression of MuSK ΔIg3 and modulation of MuSK expression in the cells of bodily fluids, organs or tissues. An effective amount of can be determined by monitoring the effect on functional MuSK ΔIg3 protein expression of MuSK-targeting oligonucleotides that are administered to a subject or contacted to a cell.

In some embodiments, a MuSK-targeting oligonucleotide or composition comprising the same, when administered to a population of cells, (e.g., comprising NSCs and/or neural progenitor cells (MPCs)), increases the number of cells that are in an activated state (e.g., active proliferation). Cells within a population can be assessed for whether they are in an activated state by known methods in the art, including e.g., an EdU assay, where EdU+ cycling cells are compared with total cell counts. In some embodiments, a MuSK-targeting oligonucleotide or composition comprising the same, when administered to a population of cells comprising NSCs, decreases the number of quiescent NSCs in the population and/or increases the number of activated NSCs.

In some embodiments, a MuSK-targeting oligonucleotide or composition comprising the same, when administered to a population of cells, (e.g., comprising satellite cells (SCs), myoblasts, myogenic progenitor cells (MPCs)), increases the number of cells that are in an activated state (e.g., active proliferation). Cells within a population can be assessed for whether they are in an activated state by known methods in the art, including e.g., an EdU assay, where EdU+cycling cells are compared with total cell counts. In some embodiments, a MuSK-targeting oligonucleotide or composition comprising the same, when administered to a population of cells comprising satellite cells, decreases the number of quiescent satellite cells in the population and/or increases the number of activated satellite cells.

In some embodiments, a MuSK-targeting oligonucleotide or composition comprising the same, when administered to a population of cells comprising NSCs and/or NPCs, increases the number of cells expressing genes associated with early neurons (e.g., Dex) and/or decreases the number of cells expressing genes associated with mature neurons (e.g., Map2), astrocytes (e.g., GFAP and S100b), and/or oligodendrocytes (e.g., CNPase and O4). In some embodiments, a MuSK-targeting oligonucleotide, when administered to a population of cells comprising NSCs and/or NPCs, increases the level of expression of genes associated with early neurons (e.g., Dex) and/or decreases the level of expression of genes associated with mature neurons (e.g., Map2), astrocytes (e.g., GFAP and S100b), and/or oligodendrocytes (e.g., CNPase and O4) in the population of cells.

In some embodiments, a population of cells comprises NSCs that have been induced to be NSCs (e.g., from stem cells such as embryonic stems cells or pluripotent stem cells).

In some embodiments, a MuSK-targeting oligonucleotide or composition comprising the same, when administered to a population of cells comprising SCs, MPCs, and/or myoblasts, increases the number of cells expressing genes or myogenic factors (e.g., Pax7, MyoD, myogenin, and MERGE) and/or decreases the number of cells expressing genes associated with the MuSK-BMP signaling pathway (e.g., RGS4, Msx2, Myf5, Ptx3, Id1). In some embodiments, a MuSK-targeting oligonucleotide or composition comprising the same, when administered to a population of cells comprising satellite cells and/or myoblasts, increases the level of expression of genes associated with myogenic factors (e.g., Pax7, MyoD, myogenin, and MERGE) and/or decreases the level of expression of genes associated the MuSK-BMP signaling pathway (e.g., RGS4, Msx2, Myf5, Ptx3, Id1) in the population of cells.

In some embodiments, a population of cells comprises satellite cells and/or myoblasts that have been induced to be satellite cells and/or myoblasts (e.g., from stem cells such as embryonic stems cells or pluripotent stem cells).

In some embodiments, a population of cells is obtained from a healthy subject. In some embodiments, a population of cells is obtained from a subject having or at risk of having e.g., AD or a disease characterized by neurodegeneration or a subject suffering from a disease or disorder such as a neuromuscular dysfunction, a neurodegenerative disorder, a cardiac disorder (e.g., myocardial infarction, cardiomyopathy), or genetic diseases characterized by muscle wasting.

In some embodiments, a MuSK-targeting oligonucleotide or composition comprising the same, when contacted with a population of cells from a subject, increases neurogenesis in a subject. In some embodiments, a MuSK-targeting oligonucleotide or composition comprising the same is contacted with the population of cells in vivo, for example, by injection into a subject. In some embodiments, a MuSK-targeting oligonucleotide or composition comprising the same is contacted with the population of cells ex vivo by obtaining a population of cells from a subject, and neurogenesis is increased when the treated cells are re-introduced into the subject.

In some embodiments, a MuSK-targeting oligonucleotide or composition comprising the same, when contacted with a population of cells from a subject, increases muscle regeneration and/or growth in a subject. In some embodiments, a MuSK-targeting oligonucleotide or composition comprising the same is contacted with the population of cells in vivo, for example, by injection into a subject. In some embodiments, a MuSK-targeting oligonucleotide or composition comprising the same is contacted with the population of cells ex vivo by obtaining a population of cells from a subject, and muscle regeneration is increased when the treated cells are re-introduced into the subject.

In some embodiments, a MuSK-targeting oligonucleotide or composition comprising the same, when administered to a subject, will increase neurogenesis and/or growth, and/or improve cognition. Examples of methods to assess these biological effects are detailed, e.g., in the below examples.

In some embodiments, a MuSK-targeting oligonucleotide or composition comprising the same, when administered to a subject, will increase muscle regeneration and/or growth, and/or neuromuscular function, and/or myogenesis. Examples of methods to assess these biological effects are detailed, e.g., in the below examples.

### Production of MuSK-Targeting Oliogonucleotides

A MuSK-targeting oligonucleotide described herein can be synthesized by standard methods known in the art, e.g., by use of an automated synthesizer. Following chemical synthesis (e.g., solid-phase synthesis using phosphoramidite method), oligonucleotide molecules can be deprotected, annealed to ds molecules, and purified (e.g., by gel electrophoresis or HPLC). Protocols for preparation of MuSK-targeting oligonucleotide oligonucleotides are known in the art.

In some embodiments, the present disclosure provides technologies for preparing chirally controlled oligonucleotides and compositions thereof. In some embodiments, the present disclosure provides technologies for preparing stereopure oligonucleotides and compositions thereof. In some embodiments, provided oligonucleotides and compositions thereof are of high purity. In some embodiments, oligonucleotides of the present disclosure are at least 50%, 60%, 70%, 75%, 80%, 85%, 90%, 95%, or 99% stereochemically pure at linkage phosphorus of chiral internucleotidic linkages. In some embodiments, oligonucleotides of the present disclosure are prepared stereoselectively and are substantially free of stereoisomers. In some embodiments, in provided compositions comprising a plurality of oligonucleotides which share the same base sequence of the same pattern of chiral linkage phosphorus stereochemistry (e.g., comprising one or more of Rp and/or Sp, wherein each chiral linkage phosphorus is independently Rp or Sp), at least 50%, 60%, 70%, 75%, 80%, 85%, 90%, 95%, or 99% of all oligonucleotides in the composition that share the same base sequence as oligonucleotides of the plurality share the same pattern of chiral linkage phosphorus stereochemistry or are oligonucleotides of the plurality. In some embodiments, in provided compositions comprising a plurality of oligonucleotides which share the same base sequence of the same pattern of chiral linkage phosphorus stereochemistry, at least 50%, 60%, 70%, 75%, 80%, 85%, 90%, 95%, or 99% of all oligonucleotides in the composition that share the same constitution as oligonucleotides of the plurality share the same pattern of chiral linkage phosphorus stereochemistry or are oligonucleotides of the plurality.

MuSK-targeting oligonucleotides can also be formed within a cell by transcription of RNA from an expression construct introduced into the cell (see, e.g., Yu et al., Proc. Natl. Acad. Sci. USA 2002:99:6047-6052). An expression construct for in vivo production of oligonucleotide molecules can include one or more antisense encoding sequences operably linked to elements necessary for the proper transcription of the antisense encoding sequence(s), including, e.g., promoter elements and transcription termination signals. Preferred promoters for use in such expression constructs include the polymerase-III HI-RNA promoter (see, e.g., Brummelkamp et al., Science 2002; 296:550-553) and the U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002 99:6047-6052). A MuSK-targeting oligonucleotide expression construct can further comprise one or more vector sequences that facilitate the cloning of the expression construct. Standard vectors that can be used include, e.g., pSilencer 2.0-U6 vector (Ambion Inc., Austin, Tex.).

### Pharmaceutical Compositions

The present disclosure provides pharmaceutical compositions that comprise and/or deliver MuSK-targeting oligonucleotides as described herein. The present disclosure also provides pharmaceutical compositions that are or comprise cell populations that have been exposed to MuSK-targeting oligonucleotides as described herein.

For example, in some embodiments, a provided pharmaceutical composition may comprise and/or deliver MuSK-targeting oligonucleotides that, when administered, achieves an increase in level and/or activity of a MuSK polypeptide (e.g., a MuSK ΔIg3 polypeptide, or another MuSK variant polypeptide with disrupted Ig3) that lacks an Ig3 domain functional for interaction with BMP. Alternatively or additionally, in some embodiments, a provided pharmaceutical composition may comprise and/or deliver a population of cells that has been exposed to a MuSK-targeting oligonucleotide, so that neuronal cell number and/or activity is increased in the population.

In many embodiments, a pharmaceutical composition will be or comprise an active agent (e.g., a MuSK-targeting oligonucleotide as described herein or a precursor thereof) in combination with one or more pharmaceutically acceptable excipients. Those skilled in the art will appreciate that components of a particular pharmaceutical composition may be influenced by route of administration of the pharmaceutical composition.

The compositions of the disclosure can be formulated for a variety of modes of administration, including systemic and topical or localized administration. Techniques and formulations generally may be found in Remington, The Science and Practice of Pharmacy, (20th ed. 2000).

Compositions of the present invention can be prepared and administered in a wide variety of oral, parenteral, and topical dosage forms. Thus, the compositions of the present invention can be administered by injection (e.g. intravenously, intramuscularly, intracutaneously, subcutaneously, intraduodenally, or intraperitoneally). Also, the compositions described herein can be administered by inhalation, for example, intranasally. Additionally, the composition of the present invention can be administered transdermally. It is also envisioned that multiple routes of administration (e.g., intramuscular, oral, transdermal) can be used to administer the compositions of the invention.

In some embodiments, a pharmaceutical composition as described herein may be formulated for delivery by a route selected from intravenous injection, intrathecal administration, oral administration, buccal administration, inhalation, nasal administration, topical administration, ophthalmic administration or otic administration. In some embodiments, a pharmaceutical composition may be formulated for delivery by intrathecal administration. In some embodiments, a pharmaceutical composition may be formulated for delivery by intravenous administration. In some embodiments, a pharmaceutical composition may be formulated for delivery by oral administration.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions, described herein, within the central nervous system may be achieved with intraparenchymal administration, intrathecal administration, or intracerebroventricular administration.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC50) by a factor of 20, 25, 30, 35, 40, 45 or 50. In certain embodiments, the pharmaceutical agent in an antisense compound as further described herein. In certain embodiments, the targeted tissue is brain tissue. In certain embodiments the targeted tissue is hippocampus tissue. In certain embodiments, decreasing EC50 is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

In addition to the active ingredients, these pharmaceutical compositions may contain suitable pharmaceutically acceptable carriers comprising excipients and auxiliaries, which facilitate processing of an active compound into preparations which can be used pharmaceutically: The preparations formulated for oral administration may be in the form of tablets, dragees, capsules, or solutions.

Pharmaceutical preparations for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol: cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, an active compound may be dissolved or suspended in suitable liquids, such as fatty oils, liquid paraffin, or liquid polyethylene glycols (PEGs). In addition, stabilizers may be added.

In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Depending on the specific conditions being treated, pharmaceutical composition of the present disclosure may be formulated into liquid or solid dosage forms and administered systemically or locally. The pharmaceutical composition may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration: parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the pharmaceutical composition of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compositions herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

In some embodiments, compositions as described herein can be formulated using pharmaceutically acceptable carriers available in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, one or more solubilizing, diluting, or dispersing substances such as, saline, preservatives, such as benzyl alcohol, absorption promoters, and fluorocarbons, may be employed.

In some embodiments, a provided composition may comprise and/or deliver a precursor of an active agent, wherein the precursor becomes or releases active therapeutic agent upon administration. In some embodiments, for example, a precursor may be or comprise a prodrug of a small molecule agonizing agent, or a nucleic acid that encodes a protein agonizing agent, etc.

In some particular embodiments, a provided pharmaceutical composition comprises or delivers a therapeutically effective amount (e.g., an amount that is effective when administered according to an established protocol) of a provided oligonucleotide (which may, as described herein, be provided in a pharmaceutically acceptable salt form, e.g., as a sodium salt, ammonium salt, etc.); in some embodiments, such a provided pharmaceutical composition includes a relevant oligonucleotide and at least one pharmaceutically acceptable inactive ingredient selected from pharmaceutically acceptable diluents, pharmaceutically acceptable excipients, and pharmaceutically acceptable carriers. In some embodiments, a salt form of a provided oligonucleotide comprises two or more cations, for example, in some embodiments, up to the number of negatively charged acidic groups (e.g., phosphate, phosphorothioate, etc.) in an oligonucleotide.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

As appreciated by a person having ordinary skill in the art, oligonucleotides may be formulated as a number of salts for, e.g., pharmaceutical uses. In some embodiments, a salt is a metal cation salt and/or ammonium salt. In some embodiments, a salt is a metal cation salt of an oligonucleotide. In some embodiments, a salt is an ammonium salt of an oligonucleotide. Representative alkali or alkaline earth metal salts include sodium, lithium, potassium, calcium, magnesium, and the like. In some embodiments, a salt is a sodium salt of an oligonucleotide. In some embodiments, pharmaceutically acceptable salts include, when appropriate, nontoxic ammonium, quaternary ammonium, and amine cations formed with counterions such as hydroxide, carboxylate, sulfate, phosphate, nitrate, sulfonate, phosphorothioate, etc. that may be within provided oligonucleotides. As appreciated by a person having ordinary skill in the art, a salt of an oligonucleotide may contain more than one cations, e.g., sodium ions, as there may be more than one anions within an oligonucleotide.

In some embodiments, provided oligonucleotides, and compositions thereof, may be effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

In some embodiments, the present disclosure provides technologies (e.g., compositions, methods, etc.) for combination therapy, for example, with other therapeutic agents and/or medical procedures. In some embodiments, provided oligonucleotides and/or compositions may be used together with one or more other therapeutic agents. In some embodiments, provided compositions comprise provided oligonucleotides, and one or more other therapeutic agents. In some embodiments, the one or more other therapeutic agents may have one or more different targets, and/or one or more different mechanisms toward targets, when compared to provided oligonucleotides in the composition. In some embodiments, a therapeutic agent is an oligonucleotide. In some embodiments, a therapeutic agent is a small molecule drug. In some embodiments, a therapeutic agent is a protein. In some embodiments, a therapeutic agent is an antibody. A number of a therapeutic agent may be utilized in accordance with the present disclosure. In some embodiments, provided oligonucleotides or compositions thereof are administered prior to, concurrently with, or subsequent to one or more other therapeutic agents and/or medical procedures. In some embodiments, provided oligonucleotides or compositions thereof are administered concurrently with one or more other therapeutic agents and/or medical procedures. In some embodiments, provided oligonucleotides or compositions thereof are administered prior to one or more other therapeutic agents and/or medical procedures. In some embodiments, provided oligonucleotides or compositions thereof are administered subsequent to one or more other therapeutic agents and/or medical procedures. In some embodiments, provide compositions comprise one or more other therapeutic agents.

### Production of Pharmaceutical Compositions

For preparing pharmaceutical compositions from the compositions of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the therapeutic agent. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active therapeutic agent with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for compositions of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compositions of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, which is herein incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

### Patient Populations

In some embodiments, an appropriate patient or population is one suffering from and/or susceptible to a disease, disorder or a condition associated with neurodegeneration (e.g., AD) or that otherwise would benefit from increased neurogenesis. In some embodiments, an appropriate patient or population is one suffering from and/or susceptible to a disease, disorder such as neuromuscular dysfunction, a cardiac disorder (e.g., myocardial infarction, cardiomyopathy), or genetic diseases characterized by muscle wasting or that otherwise would benefit from increased muscle regeneration).

In some embodiments, such neurodegenerative disease, disorder, or condition is one or more of Alzheimer's Disease (AD), Parkinson's disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy, Hippocampal Sclerosis, Niemann Pick Type C, Diabetes-mediated hippocampal neuronal loss, brain injury (e.g., traumatic and/or anoxic brain injury), and Huntington's disease.

In some embodiments, population may additionally or alternatively be suffering from and/or susceptible to a disease or disorder of the lung. In some embodiments, such a disease or disorder is one or more of idiopathic pulmonary fibrosis (IPF), acute respiratory distress syndrome (ARDS), pneumonia, and lung complications due to viral infections.

In some embodiments, an appropriate patient or population is model organisms. In some embodiments, an appropriate patient or population is humans. In some embodiments, a human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, a human is a human infant. In some embodiments, a human is a human toddler. In some embodiments, a human is a human child. In some embodiments, a human is a human adult. In yet other embodiments, a human is an elderly human.

In some embodiments, an appropriate patient or population may be characterized by one or more criterion such as age group, gender, genetic background, preexisting clinical conditions, prior exposure to therapy.

In some embodiments, an appropriate patient or population is one suffering from e.g., neuromuscular dysfunction, a neurodegenerative disorder, a cardiac disorder (e.g., myocardial infarction, cardiomyopathy), or genetic diseases characterized by muscle wasting. In some embodiments, an appropriate patient or population is one suffering that has received surgery or experienced injury, trauma and/or prolonged immobilization (e.g., from bed-rest or casting). In some embodiments, an appropriate patient or population is one suffering from sarcopenia. In some embodiments, an appropriate patient or population is one suffering from or at risk of muscle fibrosis resulting from a disease or condition including, but not limited to, trauma, heritable disease, muscle disorder, and aging. Trauma can result from, for example, radiation treatment, crush injury, laceration, and amputation. In some embodiments, an appropriate patient or population is one suffering from or at risk of heritable disease associated with muscle fibrosis such as Congenital Muscular Dystrophy, Duchenne Muscular Dystrophy, Becker's Muscular Dystrophy; Amyotrophic Lateral Sclerosis (ALS), age-associated sarcopenia, Distal muscular dystrophy, Emery-Dreifuss muscular dystrophy, Facioscapulohumeral muscular dystrophy, Limb-girdle muscular dystrophy, Myotonic muscular dystrophy, and Oculo-pharyngeal muscular dystrophy.

In some embodiments, an appropriate patient or population maybe defined by those in accordance with the screening tools for diseases or disorders associated with Alzheimer's disease. In some embodiments, an appropriate patient or population maybe defined by those in accordance with the screening tools for other diseases characterized by neurodegeneration, e.g., Parkinson's disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy, Hippocampal Sclerosis, Niemann Pick Type C, Diabetes-mediated hippocampal neuronal loss, brain injury (e.g., traumatic and/or anoxic brain injury), and Huntington's disease.

In some embodiments, an appropriate patient or population maybe defined by those in accordance with the screening tools for diseases or disorders associated with muscle fibrosis and/or muscle wasting. In some embodiments, an appropriate patient or population may be defined by those in accordance with the screening tools and methods for diagnosing a disease associated with muscle fibrosis and/or muscle wasting.

In some embodiments, an appropriate patient or population may be defined according to the results obtained in structural imaging (e.g., magnetic resonance imaging (MRI), computed tomography (CT), ultrasound etc.). In some embodiments, an appropriate patient or population may be defined according to the results of cognitive tests. In some embodiments, an appropriate patient or population may be defined according to the results of neurological tests. In some embodiments, the cognitive tests involve one or more tests of Motor Screening Task (MOT), Reaction Time (RTI), Paired Associates Learning (PAL), Spatial Working Memory (SWM), Pattern Recognition Memory (PRM), Delayed Matching to Sample (DMS), Rapid Visual Information Processing (RVP). Rapid Visual Information Processing (RVP), Delayed Matching to Sample (DMS), Match to Sample Visual Search (MTS). In some embodiments, an appropriate patient or population may be defined according to the results of assessments such as measuring muscle enzymes, EMG, muscle biopsy, genetic testing, heart testing (e.g., ECG), assessments of strength and respiratory function.

### Administration

Those skilled in the art will appreciate that, in some embodiments, dosage administered to a subject, particularly a human, may vary, for example depending on the particular therapeutic and/or formulation employed, the method of administration, the dosing regimen, one or more characteristics of the particular subject being treated, etc. In some embodiments, a clinician skilled in the art will determine the therapeutically effective amount of a therapeutic to be administered to a human or other subject in order to treat or prevent a particular medical condition. The precise amount of the therapeutic required to be therapeutically effective will depend upon numerous factors, e.g., such as the specific activity of the therapeutic, and the route of administration, in addition to many subject-specific considerations, which are within those of skill in the art.

In some embodiments, administration may be ocular, oral, buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc.

Those skilled in the art, reading the present disclosure will appreciate that, in some embodiments, it may be desirable to achieve delivery of a MuSK-targeting oligonucleotide composition to muscle. Alternatively or additionally, in some embodiments, it may be desirable to achieve delivery of a MuSK-targeting oligonucleotide composition to the CNS (e.g., the brain, such as the hippocampus and/or the subventricular region) and/or to the lung.

In some embodiments, a MuSK-targeting oligonucleotide composition is delivered via systemic delivery and/or local delivery to muscle (e.g., via intramuscular injection).

In some embodiments, a MuSK-targeting oligonucleotide composition is administered using a viral vector to effectively deliver a MuSK-targeting oligonucleotide composition in the form of a nucleic acid payload. In some embodiments, a viral vector targets certain cell types (e.g., myoblasts, myocytes, myotubes, satellite cells and myofibers). AAV1, AAV6, and AAV9 vectors have been used to target different muscle cell types (See. for example, Arnett et al., Mol Ther Methods Clin Dev. 1. pii: 14038, 2014 and Riaz et al., Skeletal Muscle 5 (37)₂₀₁₅).

Those skilled in the art, reading the present disclosure will appreciate that, in some embodiments, it may be desirable to achieve delivery of a MuSK-targeting oligonucleotide composition to the CNS, and, in some embodiments to the brain.

In some embodiments systemic administration achieves delivery to CNS (e.g., brain e.g., hippocampus and/or subventricular zone). In some embodiments, an agent (e.g., an agonizing agent or MuSK-targeting oligonucleotide) is delivered to the central nervous system (CNS), via intracerebroventricular administration.

Additionally, certain viral vectors are known to selectively target neurons, and to effectively deliver genetic payloads to the brain. For example, AAV2/1 vectors have been established to effectively deliver nucleic acid payloads (e.g., gene therapy, encoded RNAs, etc) to neuronal cells in the hippocampus. See, for example, Hammond et al PLOS One 12:e0188830, 2017: Guggenhuber et al PLOS One 5:e15707, 2010; Lawlor et al Mol. Neurodeg. 2:11, 2007). Analogously, certain AAV vectors (e.g., AAV2/1 and/or AAV4 vectors) have been established to target and effectively deliver nucleic acid payloads to certain cells in the subventricular zone cells. See, for example, Liu et al Gene Thep 12:1503, 2005; Bockstael et al Hum Gene Therap 23:doi.org/10.1089/hum.2011.216, 2012).

For subjects suffering from or susceptible to a disease, disorder or condition associated with neurodegeneration, administration that achieves delivery to the CNS, e.g., to the brain (e.g., to the hippocampus and/or the subventricular region) may be desirable.

In some embodiments, effective delivery may be achieved by systemic administration of a composition as described herein. Alternatively or additionally, in some embodiments, effective delivery may be achieved by local administration to the CNS and/or to the brain, for example by intrathecal and/or intracavitary (e.g., intracerebroventricular) delivery.

Technologies for local administration to the CNS and/or to the brain have been developed and demonstrated to be effective, for example, for various protein therapeutics (see, for example Calias et al., Pharmacol. & Therap. 144:122, 2014), for small molecules (see, for example, Dodou Pharm. J. 289:501, 2012), for cell compositions (see, for example, Eftekharzadeh et al., Iran J Basic Med Sci 18:520, 2015); and nucleic acid therapeutics (see, for example, Otsuka et al, J. Neurotrauma 28:1063, 2011: see also prescribing information for onasemnogene abeparvovec-xioi [sold under the brand name Zolgensma™] and that for nusinersen [sold under the brand name Spinraza™]).

Those skilled in the art will be aware that intrathecal delivery may be particularly effective to achieve delivery to the hippocampus, including for cellular, protein, and nucleic acid therapeutics.

Systemic administration technologies (including, e.g., oral, parenteral, mucosal, etc) are well established for a wide variety of agents. Systemic administration that achieves CNS and/or brain delivery, in some embodiments, may depend on ability to cross the blood brain barrier (BBB).

Certain active agents and/or delivery systems are known to cross the BBB. Recent technologies have been shown to achieve CNS and/or brain delivery even of agents, such as oligonucleotides, that had historically been considered to be particularly challenging in that regard. To give but one example, Min et al. Angew Chem Int Ed Engl doi: 10.1002/anie.201914751, 2020, incorporated herein by reference, describes glucose-coated polymeric nanocarriers that transport oligonucleotides across the BBB.

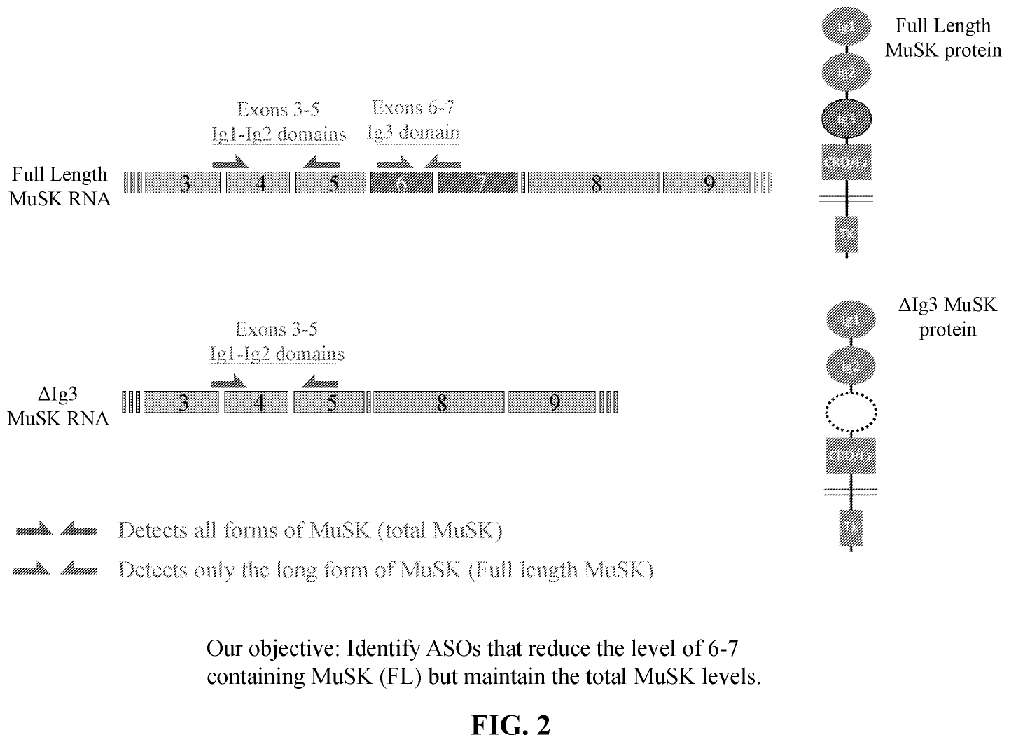

It has also been reported that incorporation of certain particular chemistries into oligonucleotide therapeutics can facilitate their travel across the BBB. For example, Khorkova et al (Nature Biotech 35:249, 2017, incorporated herein by reference) have described that:
“2′-modified phosphorothioate oligonucleotides . . . may be particularly adaptable for CNS disorders, given their long half-life, with effects in the brain lasting up to 6 months following a single injection. In another type of sugar moiety modification, locked nucleic acids (LNAs), a bridge is introduced that connects the 2′ oxygen and 4′ carbon. This modification substantially elevates the melting temperature of the LNA-DNA and LNA-RNA hybrids, thus allowing the creation of shorter ODN-based compounds with increased bioavailability and reduced manufacturing costs. A recently proposed tricyclo-DNA, a conformationally constrained oligonucleotide analog, has three additional C-atoms between C (5′) and C (3′) of the sugar (FIG. 2). This modification increases stability, hydrophobicity and RNA affinity, and improves tissue uptake and BBB permeability”.

### (Citations Omitted).

For subjects suffering from or susceptible to a disease or disorder such as idiopathic pulmonary fibrosis (IPF), acute respiratory distress syndrome (ARDS), pneumonia, and lung complications due to viral infections, administration that achieves delivery to the lungs may be desirable.

In some embodiments, oligonucleotides (e.g., antisense oligonucleotides) are developed to enhance their delivery to target site(s). As described in the art, oligonucleotide is covalently or non-covalently bound to additional chemical moieties (e.g., a carrier or ligand) to enhance the delivery. See Thomas C. Roberts et al. Nature Reviews Drug Discovery volume 19, pages 673-694 (2020), the entirety of which is incorporated herein by reference).

As appreciated by those skilled in the art, various technologies of bioconjugation can be utilized to enhance the delivery oligonucleotides to target site(s). For example, oligonucleotides can be covalently conjugated to lipids (e.g., cholesterol that facilitates interactions with lipoprotein particles in the circulation), peptides (for cell targeting and/or cell penetration), aptamers, antibodies and sugars (e.g., N-acetyl galactosamine to enhance safer delivery to the target site (See Verma, Ann Indian Acad Neurol. 2018 21 (1): 3-8. doi: 10.4103/aian.AIAN_298_17), N-acetylgalactosamine (GalNAc)).

As appreciated by those skilled in the art, lipid conjugates include, e.g., oligonucleotides bound to Cholesterol, α-tocopherol (vitamin E), long-chain (>C₁₈) fatty acids, lipoprotein particles (for example, HDL and LDL), etc.

As appreciated by those skilled in the art, conjugation of N-acetylgalactosamine (GalNAc) can enhance the uptake of oligonucleotides into target sites (e.g., hepatocytes).

As appreciated by those skilled in the art, antibody and aptamer conjugates can be used to enhance oligonucleotide delivery. Various receptors have been successfully targeted for oligonucleotide delivery, including, e.g., the HIV gp160 protein, HER2, CD7 (T cell marker), CD71 (transferrin receptor, highly expressed in cardiac and skeletal muscle) and TMEFF2. Similarly, oligonucleotides have also been conjugated with antibodies against CD44 (a neural stem cell marker), EPHA2 and EGFR193. Additionally, aptamers can be conjugated to oligonucleotide to enhance its delivery.

As appreciated by those skilled in the art, various nanocarriers can be used to enhance oligonucleotide delivery. For example, oligonucleotide can form non-covalent complex with cationic polymers (for example, polyethylenimine), dendrimers, CPPs (for example, MPG-8, PepFect6, RVG-9R228, and Xentry-KALA229) and inorganic methods (for example, calcium phosphate nanoparticles).

As appreciated by those skilled in the art, various lipoplexes and liposomes (e.g., lipid nano-particles (LNPs)) can be used to enhance oligonucleotide delivery.

In some embodiments, a MuSK-targeting oligonucleotide is modified to form a bioconjugate (e.g., conjugated with sugar, peptide, antibody, aptamer, lipid, etc.) to enhance its delivery to target site(s). In some embodiments, a MuSK-targeting oligonucleotide is formulated as lipoplexes and liposomes (e.g., lipid nano-particles (LNPs)) to enhance its delivery to target site(s).

Certain technologies have been developed to improve the efficiency of cellular delivery of ASOs to target site, e.g., muscle. For example, aminoglycosides (AGs) are shown to improve the delivery of antisense phosphorodiamidate morpholino oligomer (PMO) both in vitro and in vivo (See Wang, et al., Mol Ther Nucleic Acids. 2019; 16: 663-674, doi: 10.1016/j. omtn.2019.04.023). Short cell-penetrating peptides (CPPs) that can be either directly attached to oligonucleotides through covalent linkages or through the formation of noncovalent nanoparticle complexes can facilitate cellular uptake (See McClorey et al.: Biomedicines 2018, 6 (2), 51). ASO fatty acid conjugates are also reported to enhance the functional uptake of antisense oligonucleotide (ASO) in the muscle (See Prakash et al.: Nucleic Acids Research, 47, 2019, 6029-6044).

Those skilled in the art will be familiar with eteplirsen (ExonDys 51), an approved treatment for Duchenne muscular dystrophy (DMD), which is a third-generation phosphorodiamidate morpholino ASO.

Eteplirsen, sold under the brand name Exondys 51™, (Sarepta Therapeutics') causes exon 51 to be spliced out in pre-mRNA, restoring the reading frame in the 13% of patients with amenable frame-shifting mutations (See Crudele et al. Human Molecular Generics, Volume 28, Issue R1, pp. R102-R107, 2019).

Eteplirsen is administered via intravenous infusion over 35 to 60 minutes. In particular, its recommended dosage is 30 mg/kg body weight weekly. In a single-dose vial, the pharmaceutical composition is formulated as a 100 mg/2 ml or 500 mg/mL (50 mg/mL) solution.

Those skilled in the art will further be familiar with other delivery systems used with oligonucleotide therapeutics. For example, the first approved RNAi oligonucleotide therapeutic, Patisiran (Onpattro), which is developed by Alnylam Pharma for treatment for TTR (hereditary transthyretin amyloidosis, polyneuropathy), utilizes a nanoparticle delivery system (i.e., lipid nano-particles, LNP formulation); it also includes co-treatment with steroids and antihistamines.

Patisiran is administered via intravenous infusion. In particular, for patients weighing less than 100 kg, its recommended dosage is 0.3 mg/kg once every 3 weeks. For patients weighing 100 kg or more, the recommended dosage is 30 mg once every 3 weeks.

Other approved oligonucleotide therapeutics are typically administered via intravenous infusion to various organs, e.g., eyes, liver, skeletal muscle, spinal cord, etc.

In some embodiments, an oligonucleotide therapeutic as described herein may be administered intravenously. In some such embodiments, such oligonucleotide therapeutic may be administered according to a regimen reasonably comparable to that used for eteplirsen [sold under the brand name Exondys 51™]. In some such embodiments, such oligonucleotide therapeutic may be administered according to a regimen reasonably comparable to that used for Patisiran [sold under the brand name Onpattro].

In some embodiments a lower dose of an MuSK-targeting oligonucleotide oligonucleotide as described herein is 12 mg. In some embodiments, a total of 5 mg to 60 mg per dose of MuSK-targeting oligonucleotide is administered to a subject. In some embodiments, a total of 12 mg to 48 mg per dose of MuSK-targeting oligonucleotide is administered to a subject. In some aspects, a total of 12 mg to 36 mg per dose of MuSK-targeting oligonucleotide is administered to a subject. In some aspects, a total of 12 mg per dose of MuSK-targeting oligonucleotide is administered to a subject.

Those skilled in the art will be familiar with nusinersen [sold under the brand name Spinraza™], an antisense oligonucleotide therapeutic that targets the survival motor neuron-2 (SMN2)-directed gene transcript and is indicated for the treatment of spinal muscular atrophy (SMA) in pediatric and adult patients. Spinraza is administered intrathecally: In particular, its recommended dosage is 12 mg/5 ml (2.4 mg/mL) in a single-dose vial per administration, according to a regiment that involves four loading doses: the first three of which are administered at 14-day intervals, and the fourth of which is administered 30 days after the 3rd dose: a maintenance dose is administered once every 4 months thereafter. It is recommended that platelet count, coagulation laboratory testing, and quantitative spot urine protein testing is done at baseline, and prior to each dose.

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

An oligonucleotide therapeutic as described herein may be administered according to any of the dosing regimens described herein.

### Cell Therapy

In light of the ability of MuSK-targeting oligonucleotide compositions, as described herein, to promote neurogenesis (e.g., in cell populations that are or comprise neural progenitor cells), those skilled in the art reading the present disclosure will appreciate that, among other things, the present disclosure provides technologies for enhancing level of neural cells present in a cell population. That is, contacting an original cell population with a MuSK-targeting oligonucleotide composition as described herein can generate a resulting population with an increased level and/or percentage of neural cells as compared with that in the original population: administration of such MuSK-targeting oligonucleotide composition as described herein can achieve such increase.

Additionally, MuSK-targeting oligonucleotide compositions, as described herein, promote muscle regeneration (e.g., in cell populations that are or comprise SCs, MPCs, and/or myoblasts), and those skilled in the art reading the present disclosure will appreciate that, among other things, the present disclosure provides technologies for enhancing level of SCs, MPCs, and/or myoblasts present in a cell population. That is, contacting an original cell population with a MuSK-targeting oligonucleotide composition as described herein can generate a resulting population with an increased level and/or percentage of SCs, MPCs, and/or myoblasts as compared with that in the original population: administration of such MuSK-targeting oligonucleotide composition as described herein can achieve such increase.

In some embodiments, an original cell population may be or comprise NSCs and/or NPCs. In some embodiments, an original cell population may be or comprise SCs, MPCs, and/or myoblasts. In some embodiments, an original cell population is or comprises embryonic stems cells and/or pluripotent stem cells. In some embodiments, embryonic stems cells and/or pluripotent stem cells are or have been differentiated into neural or neural precursor cells, for example using techniques known in the art (see e.g., U.S. Pat. No. 9,631,175). In some embodiments, embryonic stems cells and/or pluripotent stem cells are or have been differentiated into myogenic progenitor cells, for example using techniques known in the art (See e.g., Miyagoe-Suzuki et al . . . , Stem Cells Int. 7824614 2017).

In some embodiments, as discussed above, administration to a cell population delivers the MuSK-targeting oligonucleotide composition such that it is exposed to (i.e., contacts) a relevant original cell population in vivo (e.g., in a human, and in particular in an adult human, for example into in the brain—e.g., the hippocampus and/or subventricular region of the brain, of such human).

In some embodiments, as discussed above, administration delivers the MuSK-targeting oligonucleotide composition such that it is exposed to (i.e., contacts) a relevant original cell population in vivo (e.g., in a human, and in particular in an adult human, for example into muscle tissue, of such human).

In some embodiments, administration in accordance with the present disclosure contacts a MuSK-targeting oligonucleotide composition with a population of cells (e.g., an original population of cells), that for example, may be or comprise neural progenitor cells, SCs, MPCs, and/or myoblasts, ex vivo. For example, in some embodiments, a MuSK-targeting oligonucleotide composition is administered ex vivo (e.g., in vitro) to a population of cells from a subject. In some embodiments, a population of cells obtained from a subject.

Oligonucleotides that direct exon skipping of MuSK transcript(s) to favor forms that lack functional Ig3, and/or that direct degradation (and/or block translation) of forms that include functional Ig3, may be utilized.

In some embodiments, a population of cells is contacted with a MuSK-targeting oligonucleotide composition and simultaneously or subsequently stimulated and/or expanded. Alternatively or additionally, a population of cells is enriched and/or selected for cells exhibiting characteristics of activated NSCs (e.g., expression of Dex) or satellite cells or for expression of myogenic factors (e.g., Pax7, MyoD, myogenin, and MERGE) or for decreased/lack of expression of genes associated with the MuSK-BMP signaling pathway (e.g., RGS4, Msx2, Myf5, Ptx3, Id1).

In some embodiments, a resulting population of cells, achieved by contacting an original population of cells with a MuSK-targeting oligonucleotide composition ex vivo is then administered to a subject. In some embodiments, a resulting population of cells is administered to a subject suffering from or susceptible to a disease or disorder such as a neuromuscular dysfunction, a neurodegenerative disorder (e.g., AD), a cardiac disorder (e.g., myocardial infarction, cardiomyopathy), or genetic diseases characterized by muscle wasting. In some embodiments, a resulting population of cells is administered to the subject from whom the original population of cells was obtained. In some embodiments, a resulting population of cells is administered to a different subject than the one from which the original population of cells was obtained: in some such embodiments, the original population was obtained from a healthy subject and the resulting population is administered to a subject suffering from or susceptible to a disease or disorder such as a neurodegenerative disorder (e.g., AD), a neuromuscular dysfunction, a cardiac disorder (e.g., myocardial infarction, cardiomyopathy), or genetic diseases characterized by muscle wasting.

In some embodiments, administering a population of cells, contacted with a MuSK-targeting oligonucleotide composition effectively treats a disease or disorder such as a neuromuscular dysfunction, a neurodegenerative disorder (e.g., AD), a cardiac disorder (e.g., myocardial infarction, cardiomyopathy), or genetic diseases characterized by muscle wasting in the subject.

In some embodiments, a population of stimulated and/or expanded NSCs, SCs, MPCs, and/or myoblasts described herein can be formulated into a cellular therapeutic. In some embodiments, a cellular therapeutic includes a pharmaceutically acceptable carrier, diluent, and/or excipient. Pharmaceutically acceptable carriers described herein, for example, vehicles, adjuvants, excipients, and diluents, are well known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

In some embodiments, a cellular therapeutic can be formulated for administration by any suitable route, such as, for example, intravenous, intratumoral, intraarterial, intramuscular, intraperitoneal, intrathecal, epidural, and/or subcutaneous administration routes. Preferably, the cellular therapeutic is formulated for a parenteral route of administration. In some embodiments, a cellular therapeutic is administered to a subject via an infusion.

In some embodiments, a cellular therapeutic suitable for parenteral administration can be an aqueous or non-aqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

In some embodiments, a single therapeutic cell described herein is capable of expanding and providing a therapeutic benefit. In some embodiments, 10² or more, e.g., 10³ or more, 10⁴ or more, 10⁵ or more, or 10⁸ or more, therapeutic cells are administered as a cellular therapeutic. Alternatively, or additionally 10¹² or less, e.g., 10¹¹ or less, 10⁹ or less, 10⁷ or less, or 10⁵ or less, therapeutic cells described herein are administered to a subject as a cellular therapeutic. In some embodiments, 10²-10⁵, 10⁴-10⁷, 10³-10⁹, or 10⁵-10¹⁰ therapeutic cells described herein are administered as a cellular therapeutic.

A dose of a cellular therapeutic described herein can be administered to a subject at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed. In some embodiments, a cellular therapeutic is administered in combination with another therapy.

### Combination Therapy

In some embodiments, a MuSK-targeting oligonucleotide therapy as described herein is administered in combination with another therapy—e.g., so that a subject is simultaneously or sequentially exposed to both therapies.

The dosage of the MuSK-targeting oligonucleotide therapy as described herein and the dosage of another therapy administered in combination, as well as the dosing schedule can depend on various parameters, including, but not limited to, the disease being treated (e.g., a neuromuscular dysfunction, a neurodegenerative disorder, a cardiac disorder, or a genetic disease characterized by muscle wasting), the subject's general health, and the administering physician's discretion.

MuSK-targeting oligonucleotide therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other therapy, to a subject in need thereof. In various embodiments MuSK-targeting oligonucleotide therapy and the other therapy are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, MuSK-targeting oligonucleotide therapy and the other therapy are administered within 3 hours. In another embodiment, MuSK-targeting oligonucleotide therapy and the other therapy are administered at 1 minute to 24 hours apart.

A synergistic combination of MuSK-targeting oligonucleotide therapy and the other therapy, might allow the use of lower dosages of one or both of these agents and/or less frequent administration of the therapies to a subject suffering from neuromuscular dysfunction, a neurodegenerative disorder, a cardiac disorder, or genetic diseases characterized by muscle wasting. A synergistic effect might result in the improved efficacy of these agents and/or the reduction of any adverse or unwanted side effects associated with the use of either agent alone.

In some embodiments, MuSK-targeting oligonucleotide therapy is administered in combination with a standard of care treatment for a relevant disease, disorder, or condition (e.g., a neuromuscular dysfunction, a neurodegenerative disorder, a cardiac disorder, or genetic diseases characterized by muscle wasting).

Therapies for DMD include deflazacort (Emflaza; PTC Therapeutics) eteplirsen (Exondys 51; Sarepta Therapeutics), Ataluren (Translarna; PTC Therapeutics), and glucocorticoids such as prednisone. In some embodiments, MuSK-targeting oligonucleotide therapy is administered in combination with one or more therapies for DMD.

Approved therapies for ALS include Radicava, Rilutek, Tiglutik, and Nuedexta. In some embodiments, MuSK-targeting oligonucleotide therapy is administered in combination with one or more therapies for ALS.

Approved therapies for cardiomyopathy include but are not limited to angiotensin II-converting enzyme (ACE) inhibitors, angiotensin II receptor blockers (ARBs) and spironolactone. In some embodiments, MuSK-targeting oligonucleotide therapy is administered in combination with one or more therapies for cardiomyopathy.

In some embodiments, MuSK-targeting oligonucleotide therapy is administered in combination with one or more therapies that relieves a symptom or characteristic of a relevant disease, disorder or condition, or of a therapy therefor. In some embodiments, MuSK-targeting oligonucleotide therapy is administered in combination with one or more other therapies that relieves a symptom or characteristic so that the side effects associated with said other therapies are relieved. In some embodiments, the side effect associated with therapy is characterized by one or more of nausea, vomiting, loss of appetite, muscle cramps and spasms, increased frequency of bowel movements, headache, confusion and dizziness, constipation, fatigue, excessive saliva and phlegm, pain, depression, sleep problems, and uncontrolled outbursts of laughing or crying.

Any therapy which is known to be useful, or which has been used, will be used or is currently being used for the treatment or prevention of neuromuscular dysfunction, a neurodegenerative disorder, a cardiac disorder, or genetic diseases characterized by muscle wasting, can be used in combination with the MuSK-targeting oligonucleotide therapy in accordance with the invention described herein.

### EXEMPLIFICATION

### Example 1: Design and Screening of MuSK-Targeting Oligonucleotides

In this Example, human MuSK-targeting oligonucleotides were designed and tested for their ability to induce exon skipping of the MuSK exon 6 and/or exon 7.

### Methods

### A. Cell Types

The choice of cell type was based on the expression of MuSK and the relevance of the cell type for the study. Two cell types were retained and tested:
1. HEK293 (ATCC, CRL-1573) is a cell line commonly used in neuroscience studies because of its ability to differentiate to neurogenic lineage (Shaw et al., 2002).
2. LHCN-M2 (Evercyte, CKHT-040-231-2) is a cell line that was shown to highly express MuSK (19.8NX according to ProteinAtlas). In HEK293, MuSK expression was variable, and sometimes not high enough to be detected with qPCR. In LHCN-M2, MuSK expression was more consistent and detectable. Therefore, LHCN-M2 was chosen.

### B. Cell Culture

LHCN-M2 were obtained from Evercyte (CKHT-040-231-2) and were cultured according to their protocol.

The cell vessels were pre-coated with 80 μl/cm² of 0.1% porcine gelatin (Sigma-Aldrich, Cat #G1890) in water for at least 4 h in 37° C. and up to one week. Before plating the cells, excess gelatin was removed.

For detachment, cells were rinsed twice with PBS (Thermofisher, 14190144) and were incubated with Trypsin-EDTA solution (Sigma, T3924-100ML) (room-temperature: 20 μl/cm²) for 2 minutes.

Once the cells are detached, about 160 μl/cm² of growth medium was added to the trypsin. Cells were detached by pipetting and diluted at the appropriate density (between ½ to ⅙). Final volume of growth medium was 240 μl/cm².

LHCN-M2 cells were grown in MyoUp medium at 37° C. in a humidified atmosphere with 5% CO₂. Cells were passaged twice a week when having reached about 30-40% confluence.

### MyoUp Medium:

DMEM (Thermofisher Cat #10566016)/M199 (Gibco, Cat #31150022) (4+1)
15% Fetal bovine serum (FBS) (Hyclone, SH30071.03)
20 mM Hepes (Sigma Aldrich, Cat #H0887)
3 μg/ml Zinc sulfate (Sigma, Cat #Z0251)
1.4 μg/ml Vitamin B12 (Sigma, Cat #V2876)
0.055 μg/ml Dexamethasone (Sigma Aldrich, Cat #D4902)
2.5 ng/ml HGF (Merck Millipore, Cat #GF116)
10 ng/ml bFGF (Peprotech, Cat #100-18C)

### C. ASO Transfection

ASOs were diluted in cell culture grade water to reach a concentration of 100 UM and were preserved at −20° C.

LHCN-M2 cells were plated in gelatin-coated 6 well plates at 35,000 cells/well in 2.25 mL. After 2 days, cells reached 60% confluency and were ready to be transfected with the ASOs and lipofectamine RNAiMAX (Invitrogen, 13778150) according to the manufacturer's protocol:

| | | Volume (μL) | |
|---|---|---|---|
| | | Oligos | |
| | | 50 nM | 100 nM |
| | | final | final |
| 1- Dilution of Lipofectamine in | Opti-MEM | 400 | |
| Opti-MEM | Lipofectamine | 24 | |
| 2- Dilution of siRNA in | Opti-MEM | 400 | 400 |
| OptiMEM | siRNA | 8 of | 8 of |
| | | 50 μM | 100 μM |
| 3- Add siRNA/Opti-MEM in | siRNA/Opti-MEM | 400 | |
| lipo-OptiMEM | Lipo/Opti-MEM | 400 | |
| 4- Incubate 5 min at RT | | | |
| 5- Add siRNA-lipo to cells | mix | 250 μL per well | |
| | siRNA/Lipo/Opti- | | |
| | MEM | | |

24 hours after transfection, cells were rinsed with PBS and the RNA was extracted.

### D. RNA Extraction

To obtain the best RNA extraction ratio, two kits were tested: Zymo Research Quick-RNA™ Miniprep Kit (cat #50444597) and Qiagen RNeasy Plus Mini Kit (cat #74136).

Cells were seeded in 6-well plates at a density of 45 000 cells/well. The RNA was extracted 2 days after seeding. The RNA concentration was measured using a nanodrop (Thermofisher, NanoDrop™ 8000 Spectrophotometer) and the results are given in Table 3.

TABLE 3
Table 3: Yield of RNA extraction from LHCN-M2
cells with ZYMO or QIAGEN extraction kits.

| ZYMO | | | QIAGEN | | |
|---|---|---|---|---|---|
| | Total | | | Total | |
| | (ug) | A260/A280 | | (ug) | A260/A280 |
| Control1 | 4.14 | 1.99 | Control1 | 2.56 | 2.08 |
| Control2 | 3.51 | 1.98 | Control2 | 2.86 | 1.98 |
| Control3 | 3.81 | 2.00 | Control3 | 3.27 | 1.9 |

With the Zymo kit, a yield of RNA extraction of 3.82 μg/well was obtained, while 2.9 μg/well was obtained with the Qiagen kit. Therefore, the Zymo kit was chosen for the study.

The Zymo Research Quick-RNA™ Miniprep Kit is used for RNA extraction (cat #50444597).

### Zymo Kit Protocol:

All steps at room temperature and centrifugation at 13,000×g for 30 seconds, unless specified.
1. Cells are lysed in 400 μL of RNA Lysis buffer using a cell scraper.
2. Lysates are vortexed for 20 s to be homogenized and centrifuged for 30 s at 13,000×g to remove the cell debris.
3. The supernatants are transferred into a Spin-Away™ Filter (yellow) in a Collection Tube and centrifuged to remove the majority of genomic DNA.
4. 0.5 volume of ethanol (95-100%) is added to the flow-through and well mixed. The samples are transferred into a Zymo-Spin™ IIICG Column (green) in a Collection Tube and centrifuged.
5. The RNA, attached to the column, is DNase treated.
   (D1) Wash the column with 400 μl RNA Wash Buffer and centrifuge. Discard the flow-through.
   (D2) In a nuclease-free tube, 5 μl DNase I (1 U/μl) is added to 75 μl DNA Digestion Buffer
   and mix. Mixture is directly added into the column matrix.
   (D3) The column incubates at room temperature (20-30° C.) for 15 minutes.
6. 400 μl of RNA Prep Buffer is added to the column and centrifuged. The flow-through is discarded.
7. 700 μl of RNA Wash Buffer is added to the column and centrifuged. The flow-through is discarded.
8. 400 μl of RNA Wash Buffer is added to the column and centrifuged for 1 min to ensure complete removal of the wash buffer. Then, the column is transferred into a 1.5 mL nuclease-free tube.
9. 100 μl of DNase/RNase-Free Water is added directly to the column matrix and centrifuged.
10. Extracted RNA is usually immediately transcripted to cDNA, or stored at −80° C. if necessary.
   cDNA Transcription:

The reverse transcriptase SuperScript™ IV VILO™ Master Mix from Thermofisher (11756050) is used 4 μL of the enzyme is added to 16 al of RNA (corresponding to ˜550-650 ng).

According to the manufacturer's protocol, the reverse transcription is obtained by the following steps:
10 min at 25° C. to anneal the primers
10 min at 50° C. to perform the reverse transcription
5 min at 85° C. to inactivate the enzyme

The cDNA is then diluted by 2 in DNase RNase free water and conserved at −20° C. until the qPCR is done.

For MuSK analyses, the cDNA was used at this concentration.

For housekeeping gene analyses, the cDNA was further diluted by 5.

E. qPCR

Two qPCR technologies were tested, TaqMan and SYBR Green.

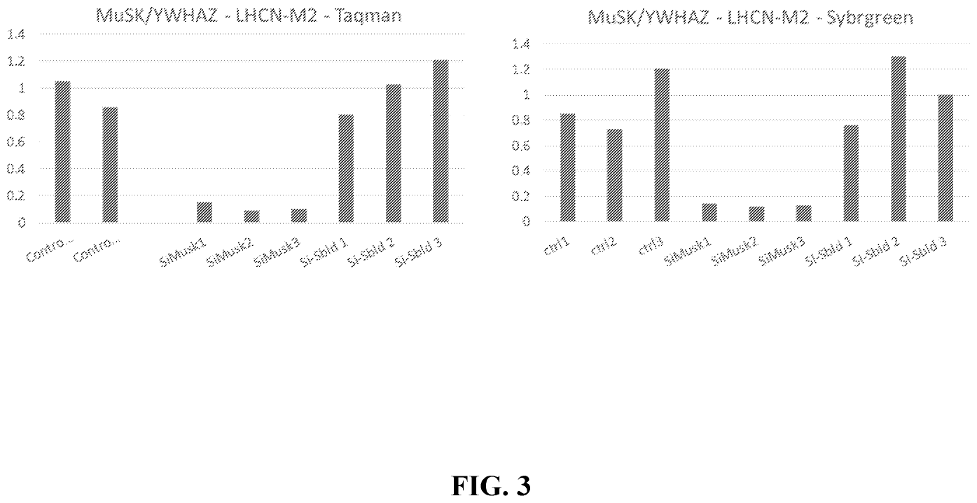

Relative MuSK expression was measured by qPCR using Taqman or SYBR green technology in LHCN-M2 cells. Both gave similar results regarding MuSK inhibition by siRNA (Thermofisher, 4392420) (FIG. 3). However, the Cq, related to the level of detection, was lower with SYBR green than for Taqman-around 22 and 27 respectively for the controls, indicating better detection with SYBR green technology. Since the level of detection is important to discriminate the level of MuSK inhibition between conditions, SYBR Green technology was chosen for the study.

SYBR Green qPCR Protocol:

To analyze the gene expression, SYBR Green technology was used. Results were normalized to two housekeeping genes (GAPDH and YWHAZ). qPCR was performed in 384-well plates. cDNA was used at ½ to analyze MuSK expression and at 1/10 to analyze HKG expressions. Measures were performed in duplicates or triplicates.

The primer sequences are shown below in Table 4:

TABLE 4

| Gene | Forward | Reverse |
|---|---|---|
| MuSK 34 (spanning | CCTGCAAGTGAAGATGAAACCTAAA | ATGAATCCTCAAGCTCCCAGA |
| the exon/exon | (SEQ ID NO: 118) | (SEQ ID NO: 119) |
| junction 3-4) | | |
| MuSK 67 (spanning | GGCTCCTGAATCCCACAATG | GAATGGACCCAGAAGAAACAGCA |
| the exon/exon | (SEQ ID NO: 120) | (SEQ ID NO: 121) |
| junction 6-7) | | |
| GAPDH (HKG) | CCTCAACGACCACTTTGTCA | TTACTCCTTGGAGGCCATGT |
| | (SEQ ID NO: 122) | (SEQ ID NO: 123) |
| YWHAZ (HKG) | CGAAGCTGAAGCAGGAGAAG | TTTGTGGGACAGCATGGATG |
| | (SEQ ID NO: 124) | (SEQ ID NO: 125) |

In each well, 9 μL of the premix [3.5 μL RNAse/DNAse free water, 5 μL SYBR Green (Biorad, 1725120), 0.5 μL primer at 10 μM] is deposited and 1 μL of the cDNA at the appropriate dilution was added. After being sealed, the plate was centrifuged, briefly vortexed and centrifuged a second time. The Thermocycler QuantStudio 6 or 7 Pro (Thermofisher) was used to perform the qPCR. The steps are:
30 s at 95° C.
PCR (X40 cycles):
   3 s at 95° C.
   30 s at 60° C.
Melting curve (step and hold, 4 s):
   20 s at 65° C.
   15 s at 95° C.

### Design and Screen of Human ASOs

A first batch of 38 human antisense oligonucleotides (ASOs) were designed and then subsequently produced by Microsynth (Switzerland). The ASO sequences are shown below in Table 5.

TABLE 5

| Oligo ID | SEQ ID | 5′ to 3′ Sequence |
|---|---|---|
| Bld1 | 1 | GCTAGGGTGGTCTTTTAGAAATGCA |
| Bld2 | 2 | GGTCAAGCTAGGGTGGTCTTTTAGA |
| Bld3 | 3 | CTGCAGGAAATGGTCAAGCTAGGGT |
| Bld4 | 4 | GAAGTGGTGAGTGACGCTCCTGCAG |
| Bld5 | 5 | GTTAGGAAGACAGAAGTGGTGAGTG |
| Bld6 | 6 | ATCCTGGCAAAAACTGTTAGGAAGA |
| Bld7 | 7 | GTGGGATTCAGGAGCCCGCAGGATC |
| Bld8 | 8 | GGTGACATTGTGGGATTCAGGAGCC |
| Bld9 | 9 | GGAGCCAAAGGTGACATTGTGGGAT |
| Bld10 | 10 | GGTCACAAAGGAGCCAAAGGTGACA |
| Bld11 | 11 | ACAGTGCAGGGTCACAAAGGAGCCA |
| Bld12 | 12 | CTGTTGCTGTACAGTGCAGGGTCAC |
| Bld13 | 13 | GGGACAGGAATGCCTGTTGCTGTAC |
| Bld14 | 14 | CAGGTGATGGTGGGGACAGGAATGC |
| Bld15 | 15 | CCGTTTTCAATCCAGGTGATGGTGG |
| Bld16 | 16 | TGACACTCACAGCATTTCCGTTTTC |
| Bld17 | 17 | AAGTCCCCACACACATGACACTCAC |
| Bld18 | 18 | GGTCTTCCCCAGACAAGTCCCCACA |
| Bld19 | 19 | ACTATGTCAGTAGATTTGAAGGGAA |
| Bld20 | 20 | TCCCACTATACTATGTCAGTAGATT |
| Bld21 | 21 | TCAGTCAAGGATTTCCCACTATACT |
| Bld22 | 22 | AAAAGAACTCAGTCAAGGATTTCCC |
| Bld23 | 23 | GTAAAGGAAAATAAAAGAACTCAGT |
| Bld24 | 24 | AACCTGACAGAGTAAAGGAAAATAA |
| Bld25 | 25 | GGACCCAGAAGAAACCTGACAGAGT |
| Bld26 | 26 | CACTCTCTTGAATGGACCCAGAAGA |
| Bld27 | 27 | CACTCGGTCTTTCACACTCTCTTGA |
| Bld28 | 28 | GTCTTGAGTCAATCACTCGGTCTTT |
| Bld29 | 29 | GATAAACAGCTGCAGTCTTGAGTCA |
| Bld30 | 30 | AGTCCTGGCTTGGTGATAAACAGCT |
| Bld31 | 31 | ATGTGTAGAGTCCTGGCTTGGTGAT |
| Bld32 | 32 | GTAGCTATGCATGTGTAGAGTCCTG |
| Bld33 | 33 | TGCTTATTGGTAGCTATGCATGTGT |
| Bld34 | 34 | ACTTCTCCCCATGCTTATTGGTAGC |
| Bld35 | 35 | CCTTGGCAGTACTGAACTTCTCCCC |
| Bld36 | 36 | CCTGCTATGCTGATGGTGGCTGCAG |
| Bld37 | 37 | GGGCATCCTACCTGCTATGCTGATG |
| Bld38 | 38 | GCAAATGTGAAGGGGCATCCTACCT |

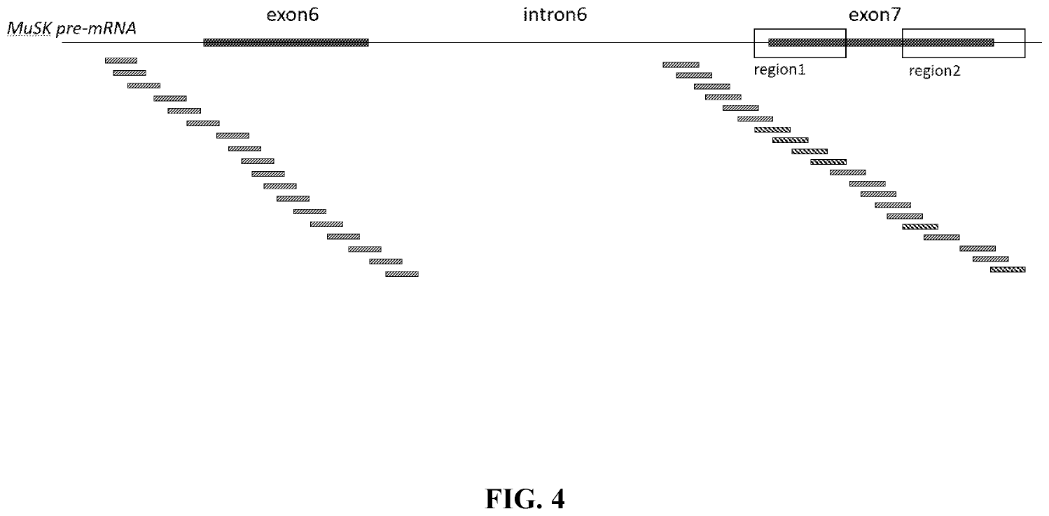

All ASOs Bld1-Bld38 were designed to include 2′-MOE modification on each sugar and where there is a phosphorothioate internucleotidic linkage between each nucleotide. Sequences were designed so that they would avoid activation of RNase H. All ASOs were 25 nucleotides in length. Alignment of ASOs Bld1-Bld38 to the MuSK genomic sequence along regions including exons 6 and 7 is shown in FIG. 4.

### Antisense Oligonucleotides Targeting Exon6

i. Alignment to the Genetic Sequence

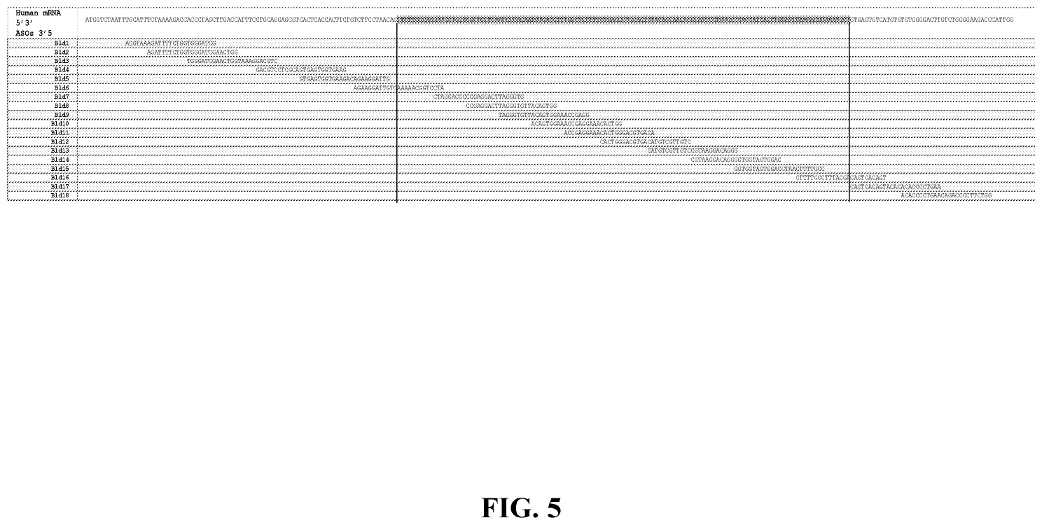

The ASOs were designed to be complementary to regions of the MuSK Exon6 genetic sequence, presented in FIG. 5.

ii. Cell Observations

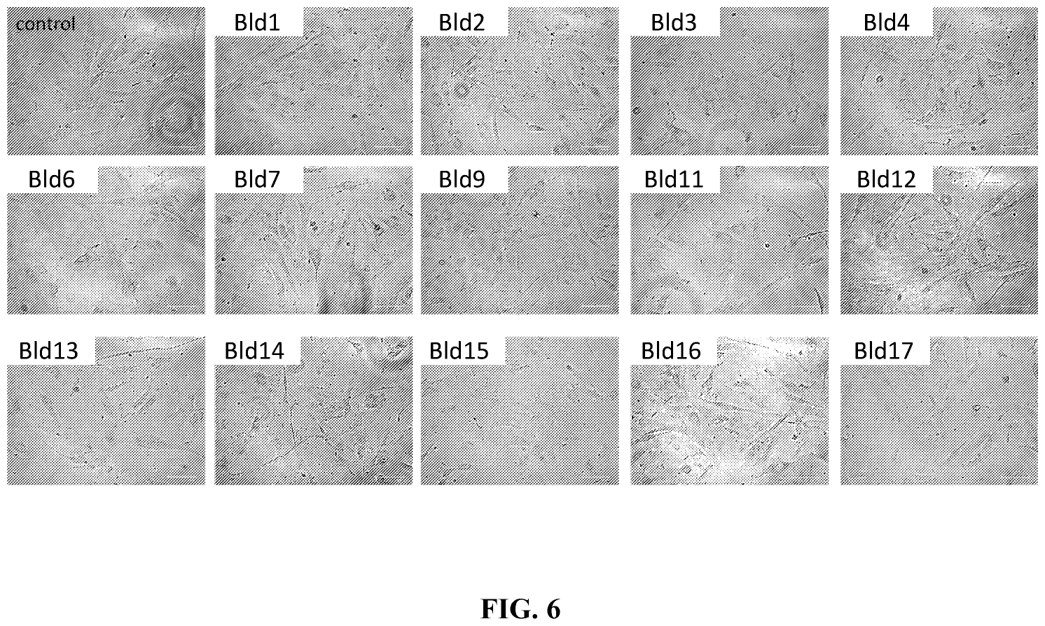

Cells were plated at 30,000 cells/well in gelatin pre-coated 6-well plates and were transfected 48 h after seeding with ASO at 50 nM and 100 nM. 24 h after transfection, cells were observed (FIG. 4) and the RNA was extracted and analyzed. FIG. 6 shows microscopic acquisitions of LHCN-M2 cells 24 h after being transfected with the ASO at 100 nM (Scale=100 nm). The data of Bld5, Bld8, Bld10, and Bld18 are not shown.

Some ASOs induced a visible effect to the cells after 24 h: Bld8 (data not shown), Bld5 (data not shown), Bld10 (data not shown), Bld126 and Bld14 appeared to have a negative effect on the cell health. Other ASOs induced small differences. For example, cells transfected with the ASOs Bld11, Bld13, and Bld15 were shorter. Otherwise, cells in the other treatment groups were healthy and similar to the control, including Bld18, the data of which are not shown in FIG. 6 and Bld9.

iii. MuSK Expression Analyses

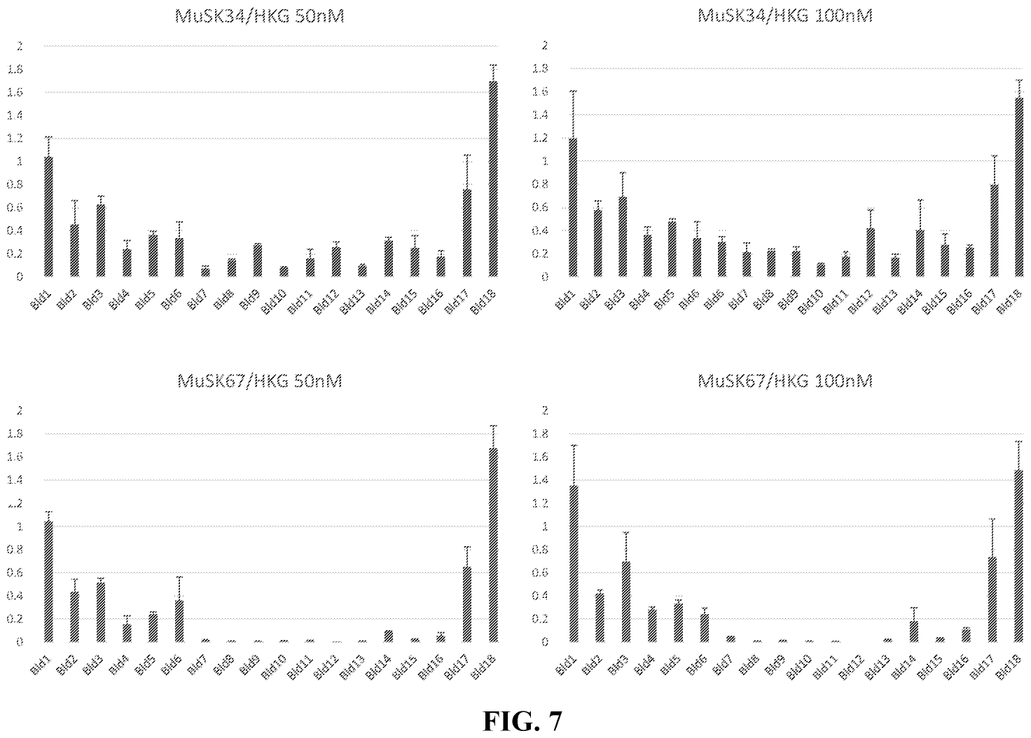

24 h after transfection, RNA was extracted and MuSK expression was measured by qPCR. The primers were designed to span either the 3-4 exon/exon junction (named MuSK34) or the 6-7 exon/exon junction (named MuSK67). The MuSK34 primer therefor detects all forms of the MuSK transcript (both the long form (i.e., containing the Ig3 domain and the short form (i.e., MuSKΔIg3)) while the MuSK67 primer detects only the long form of MuSK and not MuSKΔIg3. The primer design rationale is shown in FIG. 2. Results were normalized to two housekeeping genes, YWHAZ and GAPDH, whose stabilities were assessed using the software BestKeeper and are shown in FIG. 7.

The qPCR results show that the ASOs Bld7, Bld8, Bld9, Bld11, Bld13, Bld16, Bld15, and Bld18 induced a decrease in the expression of MuSK34 (>75%) and of MuSK67 (>90%). The ASOs Bld13, Bld4, Bld5, Bld6, Bld12 and Bld14 and Bld17 induced a decrease of MuSK34 (between 50-75%) and a higher decrease of MuSK67 (70-85%). The ASOs Bld3 and Bld17 induced slighter effects on MuSK34 and MuSK67 expression (20-50%). Finally, the two ASOs Bld1 and Bld18 induced an increase in the expression of MuSK34 and 67.

The goal of the screen of ASOs was to identify an ASO that would induce a moderate/important decrease of MuSK67 with no effect on MuSK34 (i.e., would induce an increase in MuSKΔIg3). The results shown in FIG. 7 indicate that the ASOs tested targeting Exon 6 may not be targeting the appropriate region to obtain a good skipping of Exons 6 and 7.

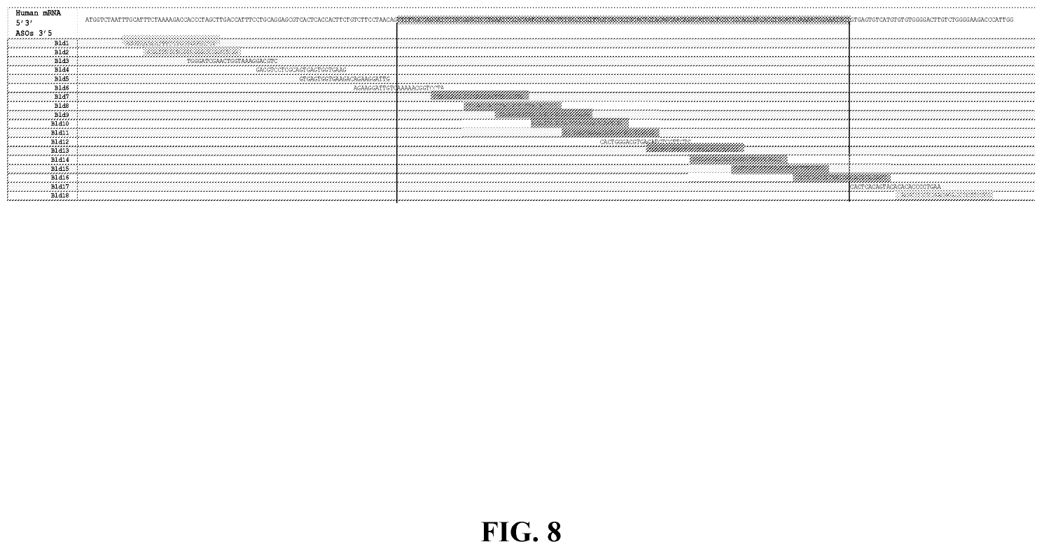

The results were correlated with the ASO alignment (FIG. 8) in order to identify key regions on the sequence important for MuSK expression. In FIG. 8, Green indicates a slight decrease or an increase in MuSK expression (between 90-300% of the control). Orange indicates a moderate decrease in MuSK expression (MuSK34 between 40 and 70% of the control, MuSK67<45% of the control). Red indicates high decrease in MuSK expression (MuSK34<40% of the control, MuSK67<20% of the control). Red areas indicate key regions for the expression of MuSK where ASOs induced a decrease of MuSK34 by >60% and MuSK67 by >80%.

The relative expression of MuSK 67 for each of the ASOs Bld1-Bld18 at 50 nM are shown in Table 6 below:

TABLE 6

| | | | |
|---|---|---|---|
| | | | Relative |
| Oligo | SEQ | | MuSK 67 |
| ID | ID NO: | Sequence-(5′ to 3′)^{a} | expression^{b} |

ASOs targeting exon 6

| | | | |
|---|---|---|---|
| Bld1 | 78 | G*C*T *A*G*G *G*T*G *G*T*C *T*T*T *T*A*G *A*A*A | 1.0449 |
| | | *T*G*C *A | |
| Bld2 | 79 | G*G*T *C*A*A *G*C*T *A*G*G *G*T*G *G*T*C *T*T*T | 0.4366 |
| | | *T*A*G *A | |
| Bld3 | 80 | C*T*G *C*A*G *G*A*A *A*T*G *G*T*C *A*A*G *C*T*A | 0.5139 |
| | | *G*G*G *T | |
| Bld4 | 81 | G*A*A *G*T*G *G*T*G *A*G*T *G*A*C *G*C*T *C*C*T | 0.1545 |
| | | *G*C*A *G | |
| Bld5 | 82 | G*T*T *A*G*G *A*A*G *A*C*A *G*A*A *G*T*G *G*T*G | 0.2419 |
| | | *A*G*T *G | |
| Bld6 | 83 | A*T*C *C*T*G *G*C*A *A*A*A *A*C*T *G*T*T *A*G*G | 0.3612 |
| | | *A*A*G *A | |
| Bld7 | 84 | G*T*G *G*G*A *T*T*C *A*G*G *A*G*C *C*C*G *C*A*G | 0.0180 |
| | | *G*A*T *C | |
| Bld8 | 85 | G*G*T *G*A*C *A*T*T *G*T*G *G*G*A *T*T*C *A*G*G | 0.0098 |
| | | *A*G*C *C | |
| Bld9 | 86 | G*G*A *G*C*C *A*A*A *G*G*T *G*A*C *A*T*T *G*T*G | 0.0091 |
| | | *G*G*A *T | |
| Bld10 | 87 | G*G*T *C*A*C *A*A*A *G*G*A *G*C*C *A*A*A *G*G*T | 0.0099 |
| | | *G*A*C *A | |
| Bld11 | 88 | A*C*A *G*T*G *C*A*G *G*G*T *C*A*C *A*A*A *G*G*A | 0.0103 |
| | | *G*C*C *A | |
| Bld12 | 89 | C*T*G *T*T*G *C*T*G *T*A*C *A*G*T *G*C*A *G*G*G | 0.0053 |
| | | *T*C*A *C | |
| Bld13 | 90 | G*G*G *A*C*A *G*G*A *A*T*G *C*C*T *G*T*T *G*C*T | 0.0088 |
| | | *G*T*A *C | |
| Bld14 | 91 | C*A*G *G*T*G *A*T*G *G*T*G *G*G*G *A*C*A *G*G*A | 0.0911 |
| | | *A*T*G *C | |
| Bld15 | 92 | C*C*G *T*T*T *T*C*A *A*T*C *C*A*G *G*T*G *A*T*G *G*T*G | 0.0222 |
| | | *G | |
| Bld16 | 93 | T*G*A *C*A*C *T*C*A *C*A*G *C*A*T *T*T*C *C*G*T *T*T*T | 0.0558 |
| | | *C | |
| Bld17 | 94 | A*A*G *T*C*C *C*C*A *C*A*C *A*C*A *T*G*A *C*A*C | 0.6501 |
| | | *T*C*A *C | |
| Bld18 | 95 | G*G*T *C*T*T *C*C*C *C*A*G *A*C*A *A*G*T *C*C*C | 1.6763 |
| | | *C*A*C *A | |

^{a}* represents a phosphorothioate linkage, and each sugar of the oligonucleotides is a 2′-MOE modified sugar.
^{b}a value of 1.0 represents the level of MuSK67 expression for untreated cells.

### Antisense Oligonucleotides Targeting Exon7

i. Alignment to the Genetic Sequence

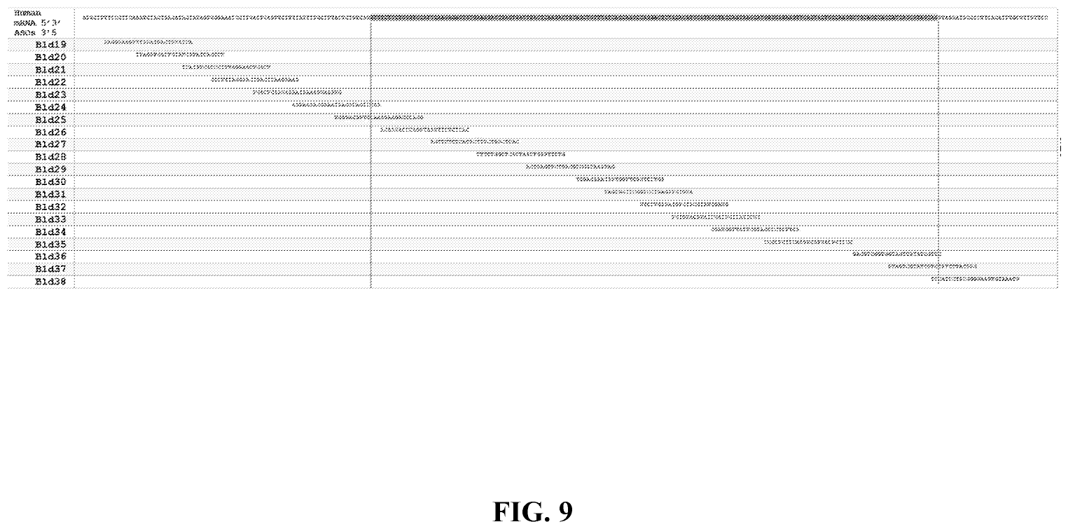

The alignment of the ASOs Bld19-Bld38 on the genetic sequence of Exon7 of MuSK is presented in FIG. 9.

ii. Cell Observations

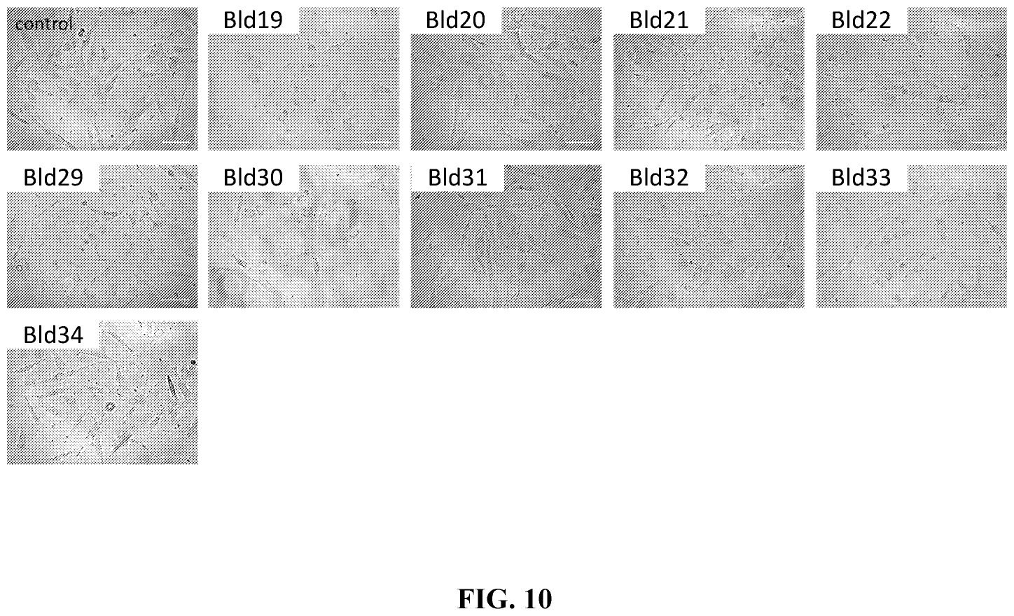

Cells were plated at 30,000 cells/well in gelatin pre-coated 6-well plates and were transfected 48 h after seeding with ASO at 50 nM and 100 nM. 24 h after transfection, cells were observed (FIG. 10) and the RNA was extracted and analyzed. FIG. 10 shows microscopic acquisitions of LHCN-M2 cells 24 h after being transfected with the ASO at 100 nM (Scale=100 nm).

After 24 h of transfection, cells looked healthy in all conditions. Cells transfected with the ASOs Bld19 and Bld30 were shorter than the control, while cells transfected with the ASOs Bld32 and Bld34 were more elongated. Cell transfected with the ASOs Bld23, Bld24, Bld25, Bld26, Bld27, Bld28, Bld35, Bld36, Bld37, Bld38 did not present any significant differences compared to the control (data not shown).

iii. MuSK Expression Analyses

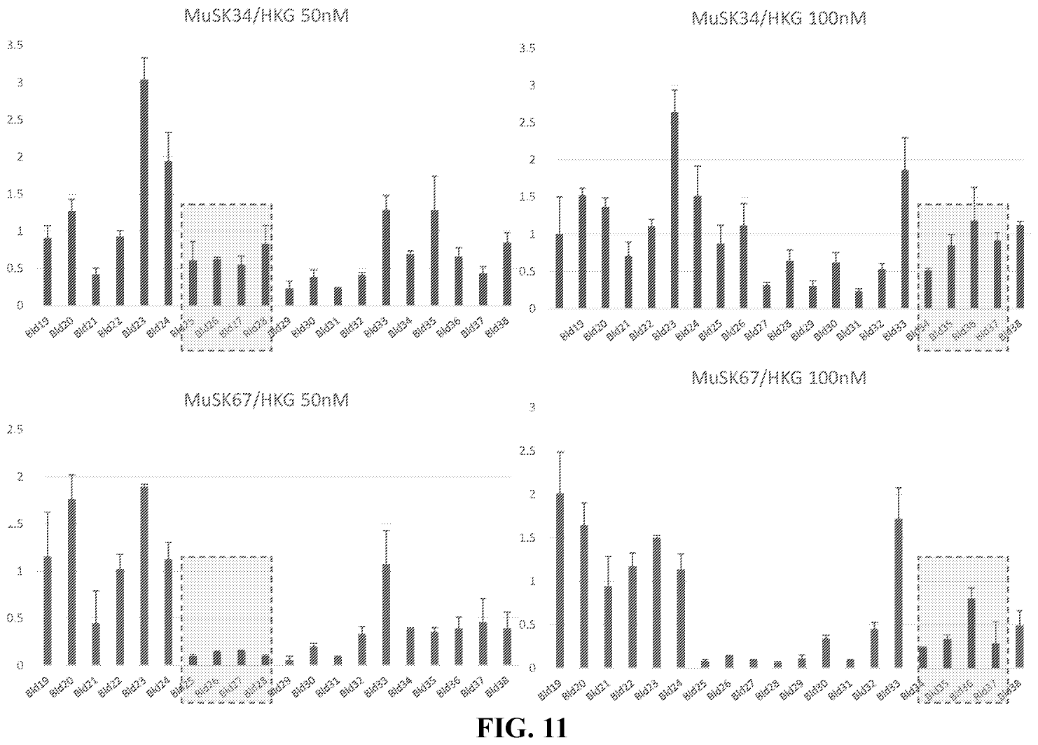

24 h after transfection, RNA was extracted, and MuSK gene expression was analyzed in qPCR (FIG. 11).Methods described for screen of ASOs to Exon6 were used.

Relative expression of each ASO at 50 nM is shown in Table 7 below:

TABLE 7

| | | | |
|---|---|---|---|
| | | | Relative |
| Oligo | SEQ | | MuSK 67 |
| ID | ID NO: | Sequence-(5′ to 3′)^{a} | expression^{b} |

ASOs targeting exon 7

| | | | |
|---|---|---|---|
| Bld19 | 96 | A*C*T *A*T*G *T*C*A *G*T*A *G*A*T *T*T*G *A*A*G | 1.1548 |
| | | *G*G*A *A | |
| Bld20 | 97 | T*C*C *C*A*C *T*A*T *A*C*T *A*T*G *T*C*A *G*T*A | 1.7641 |
| | | *G*A*T *T | |
| Bld21 | 98 | T*C*A *G*T*C *A*A*G *G*A*T *T*T*C *C*C*A *C*T*A | 0.4483 |
| | | *T*A*C *T | |
| Bld22 | 99 | A*A*A *A*G*A *A*C*T *C*A*G *T*C*A *A*G*G *A*T*T | 1.0207 |
| | | *T*C*C *C | |
| Bld23 | 100 | G*T*A *A*A*G *G*A*A *A*A*T *A*A*A *A*G*A *A*C*T | 1.8924 |
| | | *C*A*G *T | |
| Bld24 | 101 | A*A*C *C*T*G *A*C*A *G*A*G *T*A*A *A*G*G *A*A*A | 1.1254 |
| | | *A*T*A *A | |
| Bld25 | 102 | G*G*A *C*C*C *A*G*A *A*G*A *A*A*C *C*T*G *A*C*A | 0.1010 |
| | | *G*A*G *T | |
| Bld26 | 103 | C*A*C *T*C*T *C*T*T *G*A*A *T*G*G *A*C*C *C*A*G | 0.1450 |
| | | A*A*G *A | |
| Bld27 | 104 | C*A*C *T*C*G *G*T*C *T*T*T *C*A*C *A*C*T *C*T*C | 0.1570 |
| | | *T*T*G *A | |
| Bld28 | 105 | G*T*C *T*T*G *A*G*T *C*A*A *T*C*A *C*T*C *G*G*T | 0.1024 |
| | | *C*T*T *T | |
| Bld29 | 106 | G*A*T *A*A*A *C*A*G *C*T*G *C*A*G *T*C*T *T*G*A | 0.0555 |
| | | *G*T*C *A | |
| Bld30 | 107 | A*G*T *C*C*T *G*G*C *T*T*G *G*T*G *A*T*A *A*A*C | 0.1978 |
| | | *A*G*C *T | |
| Bld31 | 108 | A*T*G *T*G*T *A*G*A *G*T*C *C*T*G *G*C*T *T*G*G | 0.0871 |
| | | *T*G*A *T | |
| Bld32 | 109 | G*T*A *G*C*T *A*T*G *C*A*T *G*T*G *T*A*G* A*G*T | 0.3343 |
| | | *C*C*T *G | |
| Bld33 | 110 | T*G*C *T*T*A *T*T*G *G*T*A *G*C*T *A*T*G *C*A*T | 1.0718 |
| | | *G*T*G *T | |
| Bld34 | 111 | A*C*T *T*C*T *C*C*C *C*A*T *G*C*T *T*A*T *T*G*G | 0.3860 |
| | | *T*A*G *C | |
| Bld35 | 112 | C*C*T *T*G*G *C*A*G *T*A*C *T*G*A *A*C*T *T*C*T | 0.3559 |
| | | *C*C*C *C | |
| Bld36 | 113 | C*C*T *G*C*T *A*T*G *C*T*G *A*T*G *G*T*G *G*C*T | 0.3926 |
| | | *G*C*A *G | |
| Bld37 | 114 | G*G*G *C*A*T *C*C*T *A*C*C *T*G*C *T*A*T *G*C*T | 0.4601 |
| | | *G*A*T *G | |
| Bld38 | 115 | G*C*A *A*A*T *G*T*G *A*A*G *G*G*G *C*A*T *C*C*T | 0.3908 |
| | | *A*C*C *T | |

^{a}*represents a phosphorothioate linkage, and each sugar of the oligonucleotides is
a 2′-MOE modified sugar.
^{b}a value of 1.0 represents the level of MuSK67 expression for untreated cells.

ASOs Bld29 and Bld31 induced a decrease in the expression of MuSK34 (>75%) and of MuSK67 (>90%). ASOs Bld30, Bld32 and Bld34 induced a decrease of MuSK34 (between 40-60%) and a higher decrease of MuSK67 (55-85%). ASO Bld19 induced slighter effects on MuSK34 and MuSK67 expression (<20%). ASOs Bld19, Bld20, Bld24, Bld23 and Bld33 induced an increase in expression of MuSK34 and 67.

ASOs Bld25, Bld26, Bld27, Bld28, Bld35 and Bld38 induced a decrease of MuSK67>60% at 50 nM and a decrease of MuSK34<40% for both concentrations. Therefore, these ASOs were considered good candidates to be tested at additional doses.

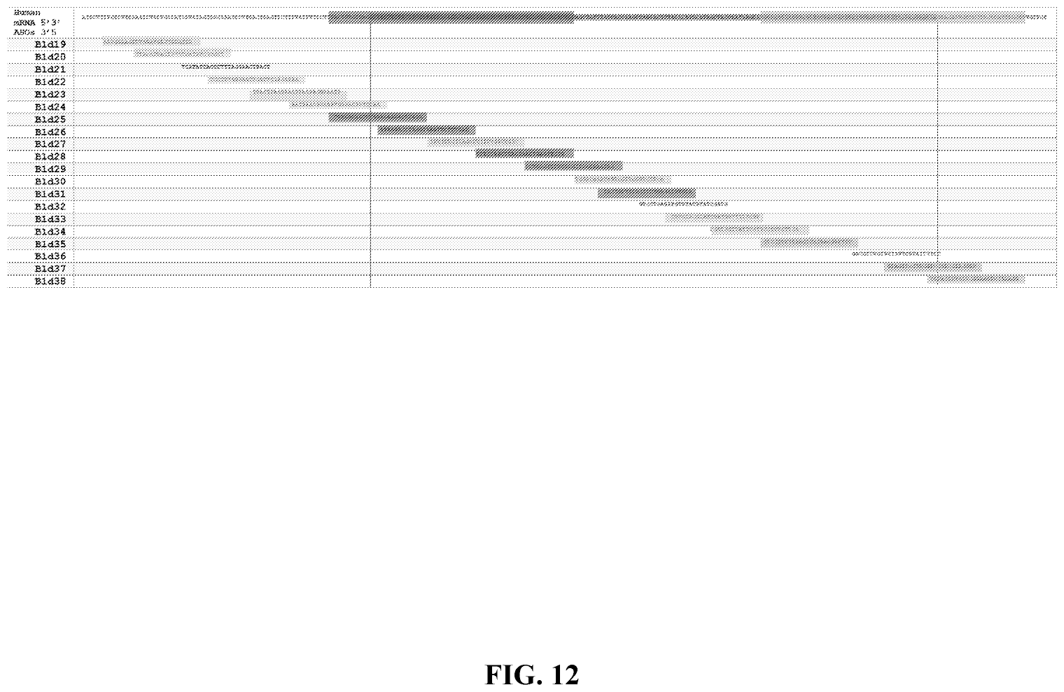

The alignment of these results to the genetic sequences indicated that one region is key region to regulate the splicing of the exons 6 and 7 without affecting the transcription of the other exons (indicated in purple in FIG. 12).

The alignment of these results to the MuSK exon7 genetic sequence indicated that some regions may be key regions to regulate the splicing of the exons 6 and 7 without affecting the transcription of the other exons (indicated in purple in FIG. 12). In FIG. 12, green indicates a slight decrease or an increase in MuSK expression (between 90-300% of the control). Orange indicates a moderate decrease in MuSK expression (MuSK34 between 40 and 70% of the control, MuSK67<45% of the control). Red indicates high decrease in MuSK expression. MuSK34<40% of the control, MuSK67<20% of the control Purple and grey areas indicate regions that may be targeted to obtain an efficient skipping of exons 6/7 with lower effect on other exons based on the results shown in FIG. 11 (i.e., the regions targeted by ASOs hu7-10, hu73, hu717, hu730, hu711 and hu7158). These regions “region 1” and “region 2” are shown in FIG. 4.

### Dose Response of Exemplary ASO Candidates

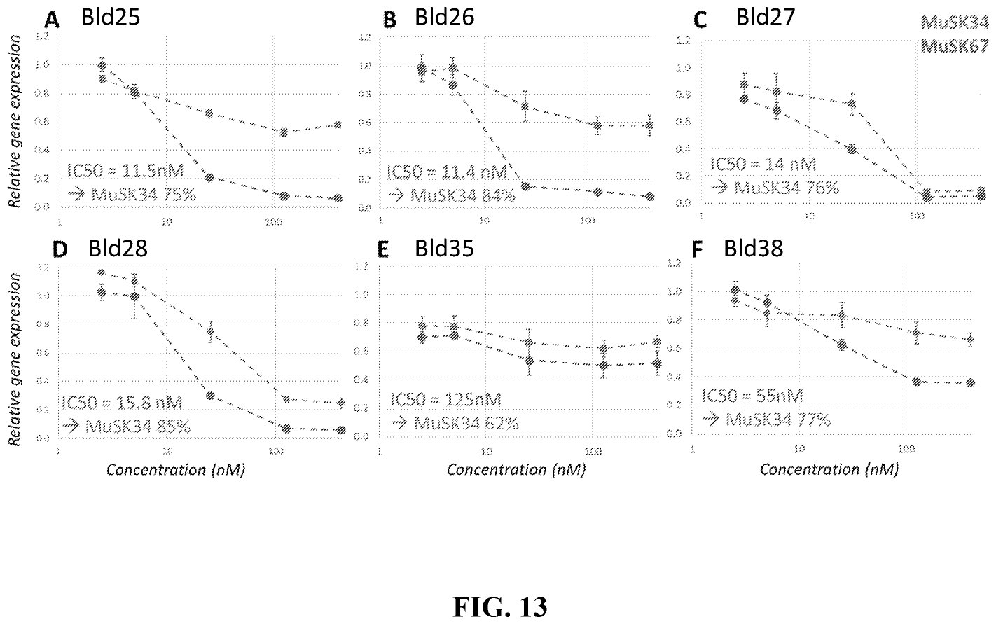

The first ASO screen at 50 and 100 nM indicated that Bld25, Bld26, Bld27, Bld28, Bld35 and Bld38 were potential candidates that could induce efficient skipping of exons 6/7 to generate MuSKΔIg3 transcript expression. These ASOs were tested at different concentrations: 2.5, 5, 25, 125 and 400 nM to examine the dose response. Results are shown in FIG. 13 (panels A-F).

Specifically, FIG. 13 shows relative MuSK expression in response to various doses of the 6 candidate ASOs (Bld25 (A), Bld26 (B), Bld27 (C), Bld28 (D), Bld35 (E), Bld38 (F)). MuSK34 (in blue) and MuSK67 (in red) expression was measured by qPCR and each was normalized to the housekeeping genes and to the values obtained for untreated cells. The estimated IC is indicated on each graph. The 5 tested doses were 2.5, 5, 25, 125, 400 nM.

The results from the dose response study indicated that ASO Bld35 induced both a decrease of MuSK34 and of MuSK67 of 20-40% and 50-70%, respectively. Therefore, the difference between the expression of MuSK34 and MuSK67 is too low to obtain a high ratio of ΔIg3 MuSK without affecting the transcription of the other exons.

For the ASOs that Bld25, Bld26, Bld27, Bld28 and Bld38, MuSK34 expression was between 75 and 85% of the control level while MuSK67 was at 50% of the control level, making them potential candidates.

For ASO Bld38, the inhibition of MuSK67 was limited at 60% of the control level, even at high concentrations (400 nM).

For ASOs Bld27 and Bld28, a concentration higher than 125 nM induced a decrease in MuSK34 expression >75%.

For ASOs Bld25 and Bld26, MuSK34 expression reached a plateau of 60% of the control level when the ASO concentration was between 25 and 400 nM, while MuSK67 expression decreased to 10% of the control. These results indicate that ASOs Bld25 and Bld26 result in a targeted decrease in expression of MuSK67, with less of an effect on MuSK34 expression, making them the 2 best candidates of this study. The estimated IC50 values were 11.5 nM and 11.4 nM for Bld25 and Bld26, respectively.

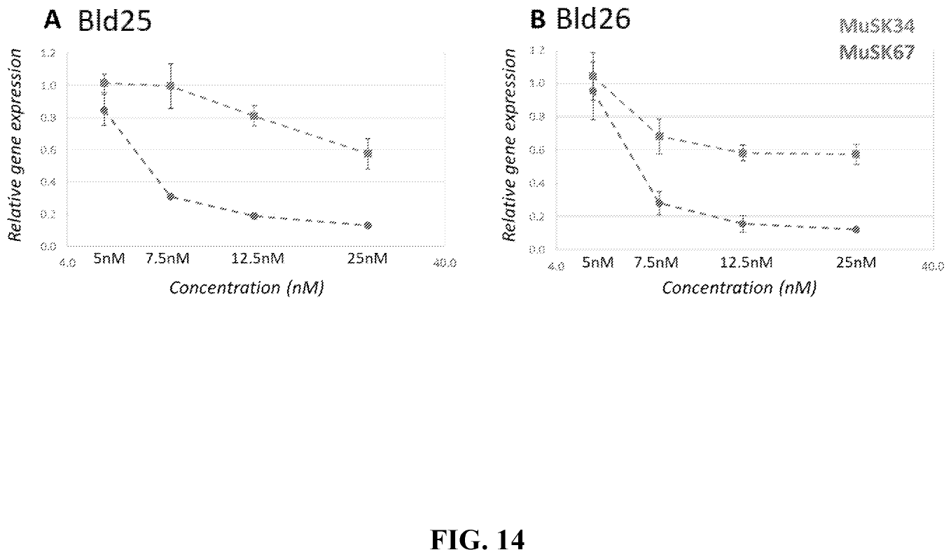

A more targeted dose response was performed in this area to refine these results (FIG. 14). These additional data indicated that the IC50 is actually between 5 and 7.5 nM for both Bld25 and Bld26. With ASO Bld26, MuSK34 expression dropped at the same ASO concentration as MuSK67 (7.5 nM). With ASO Bld25, at a concentration of 7.5 nM, MuSK67 expression decreased by 70% while MuSK34 expression was at 94% of the control. Therefore, Bld25 may be a better candidate than Bld26 to obtain a selective skipping of exons 6 and 7.

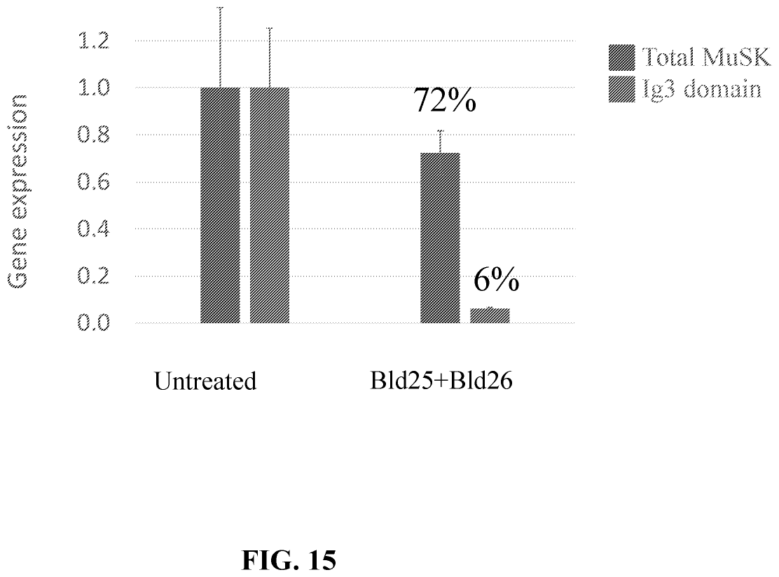

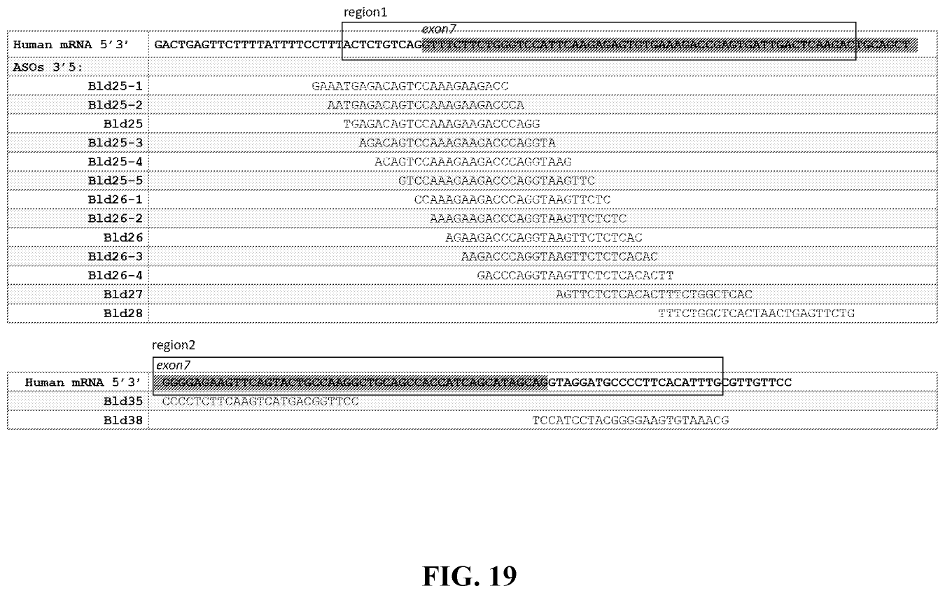

An additional test was done by combining both Bld25 and Bld26 at a final concentration of 12.5 nM. Results are shown in FIG. 15. Specifically, FIG. 19 shows MuSK34 expression in green and MuSK67 expression in red measured by qPCR and normalized to housekeeping genes and to the controls.

The Bld25/Bld26 mix resulted in MuSK34 expression at a level of 72% of the control level, while MuSK67 expression was at a level of 6% of the control level. Of note, MuSK67 expression was at 8% when inhibited by siRNA (Thermofisher, cat #4392420, Assay Id s224071) against MuSK at 10 nM (data not shown). These results suggest that combining several oligos (e.g., Bld25 and Bld26) may be a good strategy to obtain a selective skipping of exons 6 and 7.

### Confirmation of Exon Skipping by Gel Migration

To verify the ASO-induced splicing of the RNA, we amplified the genetic sequence from exon 3 to exon 9 by PCR and the size of the PCR product was measured by migrating it on gel electrophoresis.

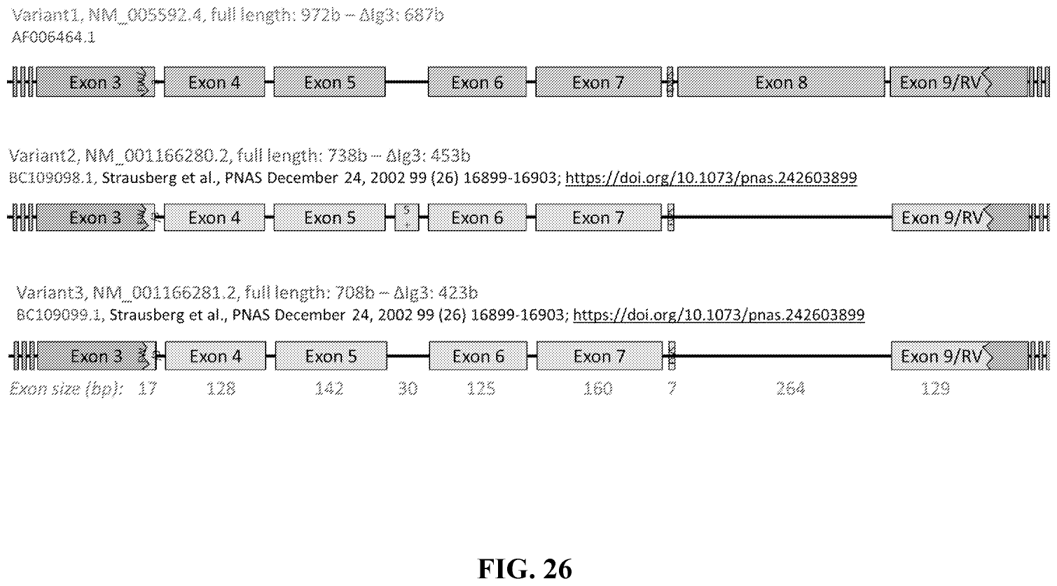

Several forms of MuSK RNA exist naturally in human. 3 variants were identified in the literature (FIG. 26):
Variant 1, NM_005592.4 (full length: 972 bp and ΔIg3: 687 bp)
Variant 2, NM_001166280.2 (full length: 783 bp and ΔIg3: 453 bp)
Variant 3, NM_001166281.2 (full length: 708 bp and ΔIg3: 423 bp)

Full length and ΔIg3 indicate the lengths of the full-length sequence and ΔIg3 (6,7) sequence amplified by PCR from exon 3 to exon 9. Compared to variant 1, the exon 8 is missing in variant 2 and variant 3. In addition, a small exon of 30 bases is present only in variant 2. Therefore, skipping exons 6 and 7 would produce 3 additional MuSK RNA forms.

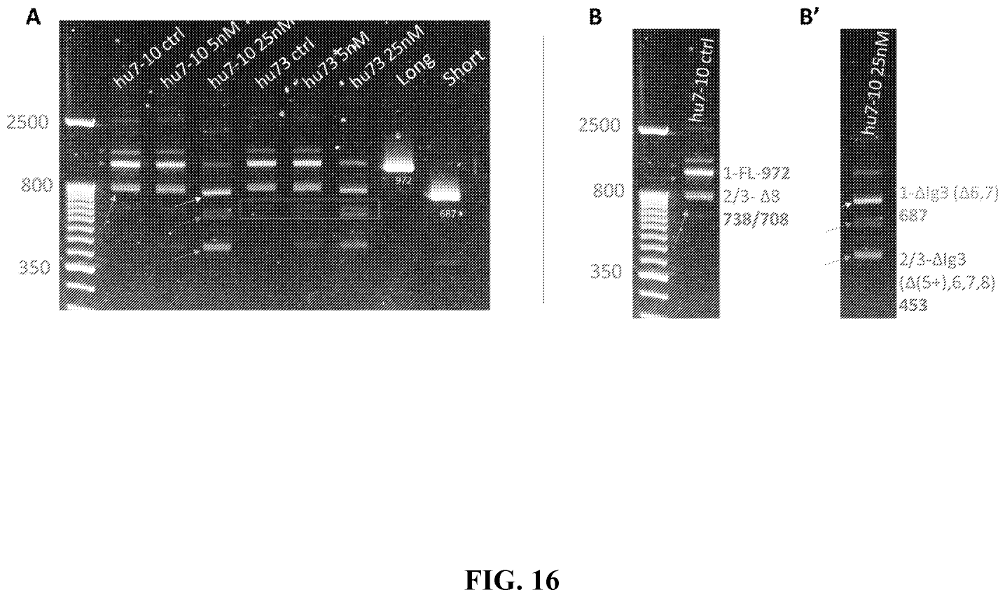

The sequence of exon3-9 of the cDNA from the dose response presented in FIG. 10 was amplified by PCR in the conditions without or with hu7-10 (Bld25) and hu73 (Bld26) at 5 and 25 nM. Results from the gel electrophoresis are shown in FIG. 16. In FIG. 16, “Long” (with exon6-7) and “Short” (without exon 6-7, i.e., ΔIg3) designed synthetic sequences were based on variant 1.

As shown in FIG. 16, several bands were detected. In the control conditions, major bands were visible at approximately 1 kb, 970b (red arrow), and a double band at 700-750b (green arrows) (see FIG. 16, panel B). In conditions where splicing of the exons 6/7 occurred (confirmed previously by qPCR), major bands were observed at approximately 690b (yellow arrow) and 450b (blue arrow) (FIG. 16, panel B′), and a lighter band at 970b. An additional double band was detected at 600b (orange arrow).

Based on the sequences presented in FIG. 16 it appears that:

The red arrow indicated the cDNA of variant 1 full-length (972 bases), while the double bands highlighted by the green arrow correspond to the variant 2 and 3 full-length (738 and 708 bases respectively). This would explain why we observed a small fraction of the red band in the Bld25 at 25 nM condition (see FIG. 16 B′) and the fraction of the green band was too light to be detected.

For the ASO hu7-10 (Bld25) at 25 nM, the yellow band corresponded to Δ6,7 variant 1 (687 bases) and the blue band corresponded to Δ5+,6,7 variant 2 and Δ6,7 variant 3 (453 bases).

Next the bands were sequenced to confirm these bands correspond to the indicated variants.

### CONCLUSIONS

This study aimed to find ASOs that would induce the skipping of the exons 6 and 7 in MuSK protein without affecting the other exons. 38 ASOs were designed and manufactured to be tested. LHCN-M2 cell line was chosen for its qPCR detectable MuSK expression.

In the first screening, we tested each of the 38 ASOs at concentrations of 50 and 100 nM. From this screen, the 6 best candidates in a key region near/on the exon 7 were selected or further testing. These 6 candidates were further tested at different doses: 2.5, 5, 25, 125 and 400 nM. The results indicated that a dose below 25 nM would be enough to obtain the desired splicing without affecting the transcription of the other exons, and that 2 ASOs, Bld25 and Bld26, were better to obtain the desired splicing, while maintaining stability of the expression of the other exons at higher doses. These 2 candidates were tested at additional doses, 5, 7.5, 12.5 and 25 nM. These results indicated that the IC50 would be between 5 and 7.5 nM for both ASOs. Additionally, Bld25 was considered the best candidate since there is a dose (7.5 nM) where we obtained an efficient skipping of exons 6/7 (by 70%) with little effect on the other exons (expression was 94% of the control level).

Additionally, a combination of these 2 candidates (Bld25 and Bld26) at 12.5 nM was also shown to be efficient in reducing the level of expression of full-length MuSK transcripts by 94% (indicated by MuSK67 in FIG. 15), where the total MuSK expression (indicated by MuSK34 in FIG. 15) was only reduced by 28%. Migration on gel electrophoresis and further sequence confirmed this was due to exon skipping.

### Example 2: In Vivo Evaluation of Exon-Skipping Induced by MuSK-Targeting Oligonucleotides

Preliminary confirmation of activities for oligonucleotides provided herein have been confirmed by testing corresponding murine oligonucleotide sequences in an established mouse model. See, e.g., Renault et al., 2009, the entirety of which is incorporated herein by reference.

In this experiment, the corresponding murine oligonucleotides are further evaluated in vivo in wild-type mice at ages where AHN has declined. Further evaluations such as pharmacodynamic and safety studies are conducted to establish optimal conditions for ASO-mediated MuSK exon skipping in aging wild type mice.

### Experiment #1: Tolerability, Exon Skipping and PK

ASOs are delivered by intracerebroventricular (ICV) stereotactic injection in 4 doses ranging from 5-100 μg, n=6/group. A scrambled ASO (with the same number of bases) will serve as a control.

Mice are monitored for potential immediate safety signals (e.g. seizures, hindlimb weakness, prolonged lethargy) and animals where these issues do not resolve within the first hours after dosing are euthanized.

Mice receiving doses that do not show tolerability signals are monitored daily.

Unfixed brains are harvested at 4 weeks. One-half of the brains are sectioned and analyzed by histology for signs of inflammation or other toxicity by a board-certified veterinary pathologist.

The hippocampus and cortex from the other half are dissected and further divided for RNA isolation and PK analysis. The level of MuSK alternative splicing (i.e., skipping of exons 6-7) are assessed by TaqMan assays and gel-based PCR as described in Examples 1-2. For the latter, gel bands are excised and sequenced to confirm correct skipping. The remaining portion for of cortex are used for HPLC quantification of the ASO (PK).

### Experiment #2. Neurogenesis (Fixed Brains)

Neurogenesis Group size: To determine the number of animals required to assess increased neurogenesis, a power analysis using G*Power version 3.1 was performed (Faul et al., 2007). To allow for possible loss of animals during the experiment 6 animals/group are allocated. Analyses is performed using either GraphPad Prism or R.

In this experiment, ASOs are delivered to a second set of animals with at least three non-toxic doses (as determined in Experiment 1).

For these animals EdU is delivered from 3 to 4 weeks post-injection. Animals are perfused, fixed and immunohistochemistry is performed against EdU and DCX to assess the numbers of new neurons (EdU+/DCX+ cells).

Maturation of the newborn cells is assessed by performing a 30-day chase after administration of the EdU and measuring Edu+/NeuN+ cells.

### Experiment #3. Cognition

Cognition group size: An analysis using G*Power 3.1.9.4. (Faul et al., 2007) was performed to determine the group size of cognition experiment. Based on the prior studies, 18 animals per group is sufficient to observe an effect size (d) of 1 (a=0.05: power=0.80).

Aged wild-type mice are dosed by ICV at 11 months of age and are assessed for cognition at 12 months of age. A single dose is performed to promote AHN as determined from Experiment #2 (18 animals per group).

The mice are evaluated using tests including Novel Object Location task, a hippocampal-dependent spatial learning task, and a Conditioned Fear and a Y-maze to measure spatial working memory (Gotz and Ittner, 2008), two tasks commonly used to test cognition.

Pairwise comparisons will be analyzed by using the Student's t test. Experiments with more than two groups will be analyzed using a Bonferroni correction for multiple comparisons.

Female mice. Efficacy of ASO treatment on promoting neurogenesis using the optimal dose determined above in female mice is also assessed.

Discussion Experiments 1-3 are designed such that they will relate the dose delivered to safety, PK, level of MuSK splicing, degree of AHN promotion and cognitive improvement.

A 4 week period of exposure was utilized since this is the time course over which NSCs are born and mature (Babcock et al., 2021). However, additionally studies with longer exposures, with and without repeat dosing will also be performed.

In addition to the HPLC measurement of ASO in the brain hybridization-based method will also be utilized.

### Example 3: In Vivo Evaluation of Exon-Skipping Induced by MuSK-Targeting Oligonucleotides in FAD Model

In this experiment, the corresponding murine oligonucleotides from Example 2 are evaluated in Familial Alzheimer's Disease (FAD) mouse model where plaque burden is well established and tangles are present. This experiments aims to determine whether corresponding murine oligonucleotides and the exon-skipping approach is effective in the hostile, inflammatory milieu along with a high plaque burden and neurofibrillary tangles that characterize the AD brain, and whether corresponding oligonucleotides promote AHN in an FAD mouse model.

FAD Mouse Model. 3×Tg is used in this experiment, which harbors transgenes for Tau, PSEN1 and APP FAD alleles (Oakley et al., 2006; Kimura and Ohno, 2009; Belfiore et al., 2019). This model has been characterized and develops both amyloid plaques at 6 months and neurofibrillary tangles by 12 months. AHN is decreased as early as 3 months of age and is essentially undetectable by 12 months. We have confirmed a reduction AHN at 6 months in these mice. This decrease in AHN is strongly correlated with the increase in amyloid burden (Rodríguez et al., 2008). Animals are dosed with ASO at 11 months and analyzed at 12 months of age, when AHN and cognitive phenotypes are evident. Female 3×Tg mice are used since they show an earlier and more consistent pathological profile (Belfiore et al., 2019).

Experimental Approach. The dosing strategy (level, duration) is based on the results from Example 3. Three groups are dosed: scrambled ASO, and two doses of MuSK exon-skipping ASOs. Group sizes will be n=18 in order to assess both AHN and cognition. 6 animals from each group are also treated with EdU once a day for 7 days for neurogenesis analysis. In order to control for potential effects of handling, the remaining animals are injected with saline. Neurogenesis and cognition analyses are performed as described in Example 3. The effects of ASO treatment on AD histopathology is also assessed.

### Example 4: Formulation and Administration of MuSK-Targeting Oligonucleotides

### A. Formulation

In this Example, human MuSK-targeting oligonucleotides as described herein are formulated into suitable forms to enhance the delivery to target site(s) and are administered to human subject via various means.

In some embodiments, human MuSK-targeting oligonucleotides are formulated into lipid complex, as a homogeneous solution in a single-dose vial. In some embodiments, single-dose vial is 10 mg/5 ml (2 mg/mL).

### B. Administration

### 1. Indication

MuSK-targeting oligonucleotides are indicated for the treatment of the AD in adults.

### 2. Dosage and Administration

### 2.1 Dosing Information

MuSK-targeting oligonucleotides are administered by a healthcare professional.

MuSK-targeting oligonucleotides are administered via intravenous (IV) infusion. Dosing is based on actual body weight. For patients weighing less than 100 kg, the recommended dosage is 0.3 mg/kg once every 3 weeks. For patients weighing 100 kg or more, the recommended dosage is 30 mg once every 3 weeks.

Missed Dose: If a dose is missed, administer MuSK-targeting oligonucleotides as soon as possible. If MuSK-targeting oligonucleotides are administered within 3 days of the missed dose, continue dosing according to the patient's original schedule. If MuSK-targeting oligonucleotides are administered more than 3 days after the missed dose, continue dosing every 3 weeks thereafter.

### 2.2 Preparation

MuSK-targeting oligonucleotides are filtered and diluted prior to intravenous infusion. The diluted solution for infusion is prepared by a healthcare professional using aseptic technique as follows:
1. Remove MuSK-targeting oligonucleotides vial from the refrigerator and allow to warm to room temperature. Do not shake or vortex.
2. Inspect visually for particulate matter and discoloration. Do not use if discoloration or foreign particles are present.
3. Calculate the required dose of MuSK-targeting oligonucleotides based on the recommended weight-based dosage [see Dosage and Administration (2.1)].
4. Withdraw the entire contents of one or more vials into a single sterile syringe.
5. Filter MuSK-targeting oligonucleotides through a sterile 0.45 micron polyethersulfone (PES) syringe filter into a sterile container.
6. Withdraw the required volume of filtered MuSK-targeting oligonucleotides from the sterile container using a sterile syringe.
7. Dilute the required volume of filtered MuSK-targeting oligonucleotides into an infusion bag containing 0.9% Sodium Chloride Injection, USP for a total volume of 200 mL. Use infusion bags that are di(2-ethylhexyl) phthalate-free (DEHPfree).
8. Gently invert the bag to mix the solution. Do not shake. Do not mix or dilute with other drugs.
9. Discard any unused portion of MuSK-targeting oligonucleotides.
10. The diluted solution should be administered immediately after preparation. If not used immediately, store in the infusion bag at room temperature (up to 30° C. [86° F.]) for up to 16 hours (including infusion time). Do not freeze.

### 2.3 Infusion

MuSK-targeting oligonucleotides are infused as follows:
1. Use a dedicated line with an infusion set containing a 1.2 micron polyethersulfone (PES) in-line infusion filter. Use infusion sets and lines that are DEHP-free.
2. Infuse the diluted solution of MuSK-targeting oligonucleotides intravenously, via an ambulatory infusion pump, over approximately 80 minutes, at an initial infusion rate of approximately 1 mL/min for the first 15 minutes, then increase to approximately 3 mL/min for the remainder of the infusion.
3. Administer only through a free-flowing venous access line. Monitor the infusion site for possible infiltration during drug administration. Suspected extravasation should be managed according to local standard practice for non-vesicants.
4. Observe the patient during the infusion.
5. After completion of the infusion, flush the intravenous administration set with 0.9% Sodium Chloride Injection, USP to ensure that all ONPATTRO has been administered.

### REFERENCES

Shaw, G., Morse, S., Ararat, M., & Graham, F. L. (2002). Preferential transformation of human neuronal cells by human adenoviruses and the origin of HEK 293 cells. The FASEB Journal: Official Publication of the Federation of American Societies for Experimental Biology, 16(8), 869-871

### Example 5: Design and Screening of 20-Mer MuSK-Targeting Oligonucleotides

In this Example, additional human MuSK-targeting oligonucleotides were designed based on the lead candidates from Example I targeting exon 7 of MuSK. The screen in Example 1 identified 6 lead ASOs that targeted two regions within the MuSK exon 7. Upon further dose response experiments and analysis of the resulting MuSK transcripts, one of these regions was identified as a key region to modulate the splicing of Ig3 domain. This region, or “region 1” corresponds to the core sequence SEQ ID NO: 126 (ACTCTGTCAGGTTTCTTCTGGGTCCATTCAAGAGAGTGTGAAAGACCGAGTGATTGACTCA AGAC) which is located at positions 83841-83905 of the MuSK transcript (SEQ ID NO: 77) (or corresponding regions within other MuSKvariant sequences). In this Example, additional ASOs are designed to target the region corresponding to SEQ ID NO: 126 (“region 1”) of exon 7 and specifically to include different portions of the sequences of ASOs Bld25 and Bld26. These ASOs were designed to test whether shorter ASOs of 20 nucleotides, rather then 25 nucleotides (as tested in Example 1) would have increased activity.

The additional oligonucleotides were tested for their ability to induce exon skipping of the MuSK exon 6 and/or exon 7.

### Design of Human ASOs

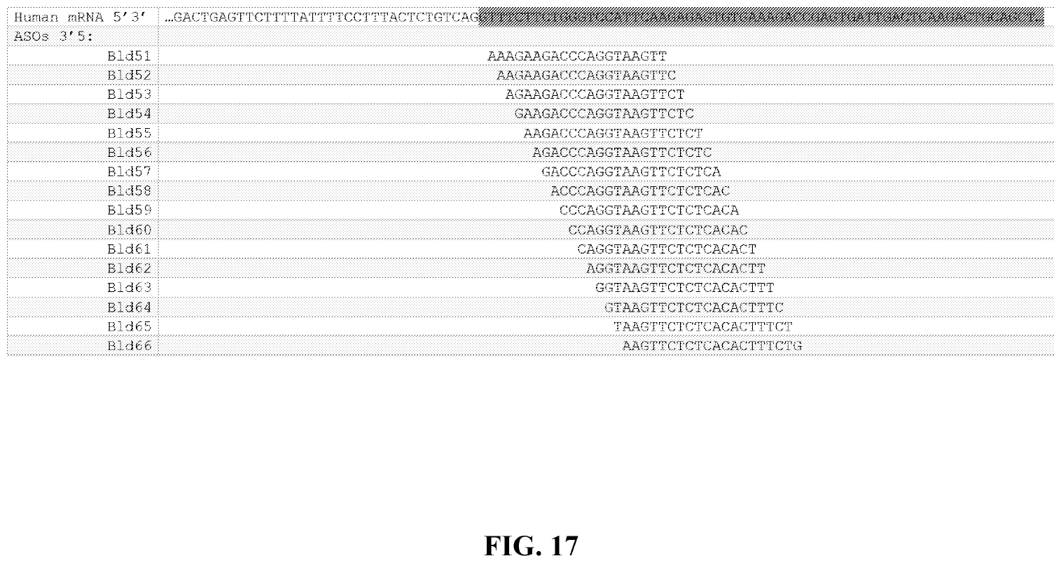

Human antisense oligonucleotides (ASOs) were designed as described above and as shown in FIG. 17, and produced by Microsynth (Switzerland). The ASO sequences are shown below in Table 8.

TABLE 8

| Oligo ID | SEQ ID | 5′ to 3′ Sequence |
|---|---|---|
| Bld51 | 127 | TTGAATGGACCCAGAAGAAA |
| Bld52 | 128 | CTTGAATGGACCCAGAAGAA |
| Bld53 | 129 | TCTTGAATGGACCCAGAAGA |
| Bld54 | 130 | CTCTTGAATGGACCCAGAAG |
| Bld55 | 131 | TCTCTTGAATGGACCCAGAA |
| Bld56 | 132 | CTCTCTTGAATGGACCCAGA |
| Bld57 | 133 | ACTCTCTTGAATGGACCCAG |
| Bld58 | 134 | CACTCTCTTGAATGGACCCA |
| Bld59 | 135 | ACACTCTCTTGAATGGACCC |
| Bld60 | 136 | CACACTCTCTTGAATGGACC |
| Bld61 | 137 | TCACACTCTCTTGAATGGAC |
| Bld62 | 138 | TTCACACTCTCTTGAATGGA |
| Bld63 | 139 | TTTCACACTCTCTTGAATGG |
| Bld64 | 140 | CTTTCACACTCTCTTGAATG |
| Bld65 | 141 | TCTTTCACACTCTCTTGAAT |
| Bld66 | 142 | GTCTTTCACACTCTCTTGAA |

All ASOs in Table 8 were designed to include 2′-MOE modification on each sugar and where there is a phosphorothioate internucleotidic linkage between each nucleotide. All ASOs in Table 8 were 20 nucleotides in length.

### Screen of Shorter Antisense Oligonucleotides Targeting Exon7 Region 1

ASO transfection and cell culture, RNA extraction, cDNA transcription, qPCR were performed according to the methods described in Example 1, unless indicated otherwise.

i. Alignment to the Genetic Sequence

The alignment of the ASOs in Table 8 on the genetic sequence of Exon7 of MuSK is presented in FIG. 17.

ii. Cell Observations

LHCN-M2 cells were plated at 30,000 cells/well in gelatin pre-coated 6-well plates and were transfected 48 h after seeding with ASO at 12.5 nM and 100 nM. 24 h after transfection, cells were observed (data not shown) and the RNA was extracted and analyzed.

After 24 h of transfection, cells looked healthy in all conditions except for cells treated with ASOs Bld54, Bld55, and Bld56 at the 100 nM dose (data not shown).

iii. MuSK Expression Analyses

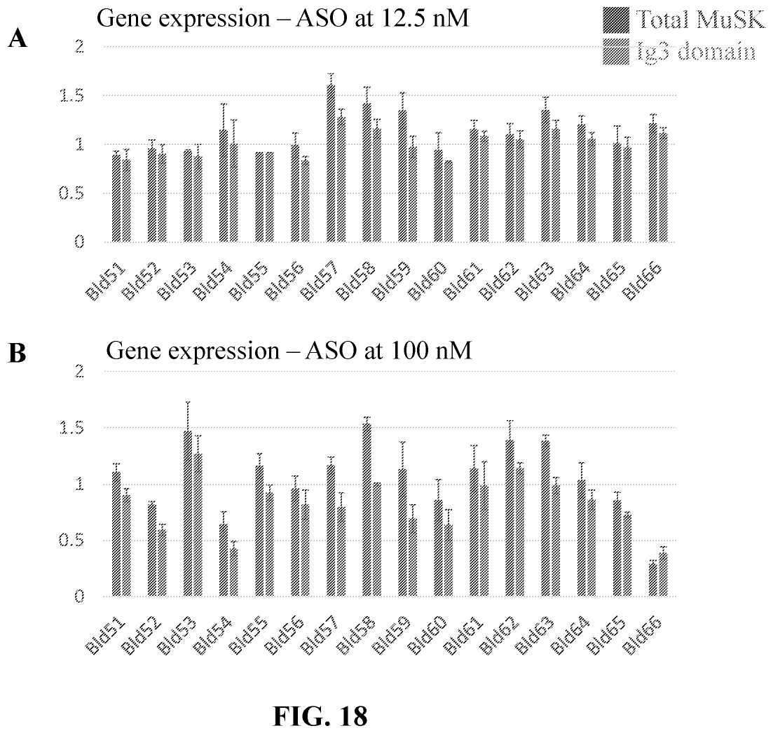

24 h after transfection, RNA was extracted, and MuSK gene expression was analyzed in qPCR. Relative gene expression of total MuSK (MuSK34) and MuSKIg3 domain (MuSK67) is shown in FIG. 18. Methods described for screening of ASOs in Example 1 were used.

ASOs with the modifications used in this Example are shown in Table 9 below.

TABLE 9

| | SEQ | |
|---|---|---|
| Oligo | ID | |
| ID | NO: | Sequence-(5′ to 3′)ª |
| Bld51 | 143 | T*T*G*A*A*T*G*G*A*C*C*C*A*G*A*A*G*A*A*A |
| Bld52 | 144 | C*T*T*G*A*A*T*G*G*A*C*C*C*A*G*A*A*G*A*A |
| Bld53 | 145 | T*C*T*T*G*A*A*T*G*G*A*C*C*C*A*G*A*A*G*A |
| Bld54 | 146 | C*T*C*T*T*G*A*A*T*G*G*A*C*C*C*A*G*A*A*G |
| Bld55 | 147 | T*C*T*C*T*T*G*A*A*T*G*G*A*C*C*C*A*G*A*A |
| Bld56 | 148 | C*T*C*T*C*T*T*G*A*A*T*G*G*A*C*C*C*A*G*A |
| Bld57 | 149 | A*C*T*C*T*C*T*T*G*A*A*T*G*G*A*C*C*C*A*G |
| Bld58 | 150 | C*A*C*T*C*T*C*T*T*G*A*A*T*G*G*A*C*C*C*A |
| Bld59 | 151 | A*C*A*C*T*C*T*C*T*T*G*A*A*T*G*G*A*C*C*C |
| Bld60 | 152 | C*A*C*A*C*T*C*T*C*T*T*G*A*A*T*G*G*A*C*C |
| Bld61 | 153 | T*C*A*C*A*C*T*C*T*C*T*T*G*A*A*T*G*G*A*C |
| Bld62 | 154 | T*T*C*A*C*A*C*T*C*T*C*T*T*G*A*A*T*G*G*A |
| Bld63 | 155 | T*T*T*C*A*C*A*C*T*C*T*C*T*T*G*A*A*T*G*G |
| Bld64 | 156 | C*T*T*T*C*A*C*A*C*T*C*T*C*T*T*G*A*A*T*G |
| Bld65 | 157 | T*C*T*T*T*C*A*C*A*C*T*C*T*C*T*T*G*A*A*T |
| Bld66 | 158 | G*T*C*T*T*T*C*A*C*A*C*T*C*T*C*T*T*G*A*A |

ª*represents a phosphorothioate linkage, and each sugar
of the oligonucleotides is a 2′-MOE modified sugar.
^{b}a value of 1.0 represents the level of MuSK67
expression for untreated cells.

Results from the screen showed that these shorter ASOs (20-mers) targeting region 1 of Exon 7 resulted in less potent activity than the ASOs Bld25 and Bld26 (25mers) and had either little/no effect on MuSKdelIg3 level or inhibited levels of total MuSK.

### Example 6: Design and Screening of Additional MuSK-Targeting Oligonucleotides Targeting Region 1 of Exon 7

In this Example, additional human MuSK-targeting oligonucleotides were designed based on the lead candidates from Example 1 targeting region 1 of exon 7 of MuSK SEQ ID NO: 126 (CTCTGTCAGGTTTCTTCTGGGTCCATTCAAGAGAGTGTGAAAGACCGAGTGATTGACTCAA GAC), which is located at positions 83841-83905 of the MuSK transcript (SEQ ID NO: 77) (or corresponding regions within other MuSK variant sequences).

Sequences were designed to specifically to include different portions of the sequences of ASOs Bld25 and Bld26. Because shortening the length of the ASOs targeting this region was showed less activity, ASOs designed in this Example were produced to be 25 nucleotides in length.

The additional oligonucleotides were tested for their ability to induce exon skipping of the MuSK exon 6 and/or exon 7.

### Design of Human ASOs

Human antisense oligonucleotides (ASOs) were designed as described above and are shown in FIG. 19. These ASOs were produced by Microsynth (Switzerland). The ASO sequences are shown below in Table 10.

TABLE 10

| Oligo ID | SEQ ID | 5′ to 3′ Sequence |
|---|---|---|
| Bld25-1 | 159 | CCAGAAGAAACCTGACAGAGTAAAG |
| Bld25-2 | 160 | ACCCAGAAGAAACCTGACAGAGTAA |
| Bld25-3 | 161 | ATGGACCCAGAAGAAACCTGACAGA |
| Bld25-4 | 162 | GAATGGACCCAGAAGAAACCTGACA |
| Bld25-5 | 163 | CTTGAATGGACCCAGAAGAAACCTG |
| Bld26-1 | 164 | CTCTTGAATGGACCCAGAAGAAACC |
| Bld26-2 | 165 | CTCTCTTGAATGGACCCAGAAGAAA |
| Bld26-3 | 166 | CACACTCTCTTGAATGGACCCAGAA |
| Bld26-4 | 167 | TTCACACTCTCTTGAATGGACCCAG |

All ASOs in Table 10 were designed to include 2′-MOE modification on each sugar and where there is a phosphorothioate internucleotidic linkage between each nucleotide. All ASOs in Table 10 were 25 nucleotides in length.

### Screen of Antisense Oligonucleotides Targeting Exon7 Region 1

ASO transfection and cell culture, RNA extraction, cDNA transcription, qPCR were performed according to the methods described in Example 1, except as where indicated otherwise.

i. Alignment to the Genetic Sequence

The alignment of the ASOs in Table 10 on the genetic sequence of Exon7 of MuSK is presented in FIG. 19.

ii. Cell Observations

LHCN-M2 cells were plated at 35,000 cells/well in gelatin pre-coated 6-well plates and were transfected 48 h after seeding with ASO at 5 nM, 12.5 nM, 25 nM, 50 nM, and 125 nM. Each of the ASOs in Table 10 were tested with Bld25, Bld26, Bld27, Bld28, Bld35 and Bld38 (leads identified in Example 1). 24 h after transfection, cells were observed (data not shown) and the RNA was extracted and analyzed.

After 24 h of transfection, cells looked healthy in all conditions except for cells treated with ASOs Bld25-3 and Bld25-4 at the 140 nM dose (data not shown).

iii. MuSK Expression Analyses

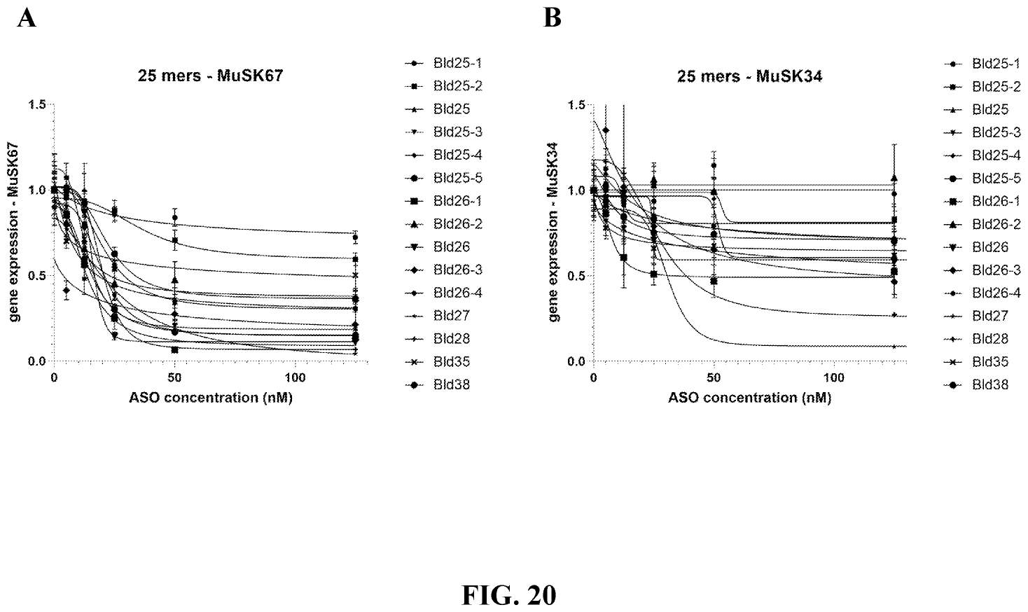

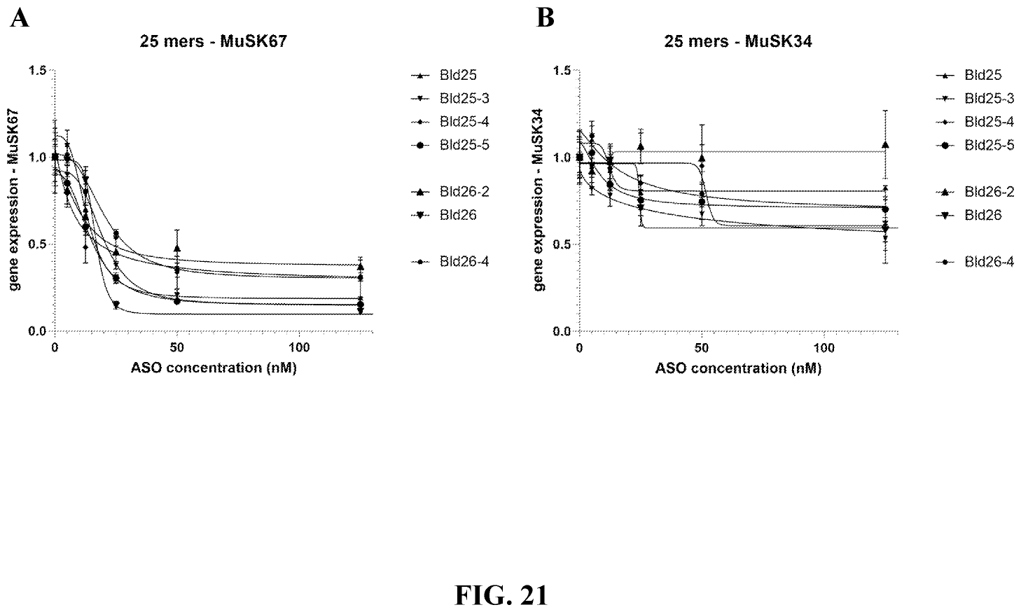

24 h after transfection, RNA was extracted, and MuSK gene expression was analyzed in qPCR (FIG. 20). Panel A shows relative gene expression of MuSK67 and Panel B shows relative gene expression of total MuSK (MuSK34). The new sequences were compared to Bld25 and Bld26 from Example 1. FIG. 21 shows the same data from only the ASOs which showed relative expression of MuSK67 of less than 50% and total MuSK (MuSK34) of greater than 60% compared to the untreated control.

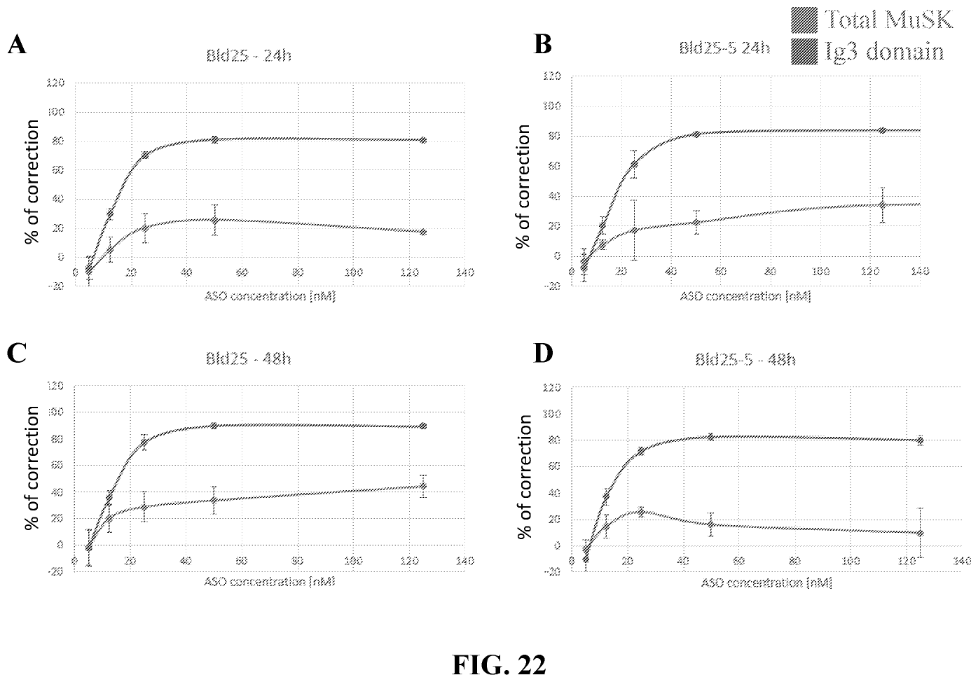

FIG. 22 shows a comparison of Bld25-5 to Bld25 after transfection with ASO for 24 hours (Panels A and B) and 48 hours (Panels C and D). Methods described for screen of ASOs in Example 1 were used.

ASOs with the modifications used in this Example are shown in Table 11 below:

TABLE 11

| Oligo ID | SEQ ID NO: | Sequence-(5′ to 3′)^{a} |
|---|---|---|
| Bld25-1 | 168 | C*C*A*G*A*A*G*A*A*A*C*C*T*G*A*C*A*G*A*G*T*A*A*A*G* |
| Bld25-2 | 169 | A*C*C*C*A*G*A*A*G*A*A*A*C*C*T*G*A*C*A*G*A*G*T*A*A* |
| Bld25-3 | 170 | A*T*G*G*A*C*C*C*A*G*A*A*G*A*A*A*C*C*T*G*A*C*A*G*A* |
| Bld25-4 | 171 | G*A*A*T*G*G*A*C*C*C*A*G*A*A*G*A*A*A*C*C*T*G*A*C*A* |
| Bld25-5 | 172 | C*T*T*G*A*A*T*G*G*A*C*C*C*A*G*A*A*G*A*A*A*C*C*T*G |
| Bld26-1 | 173 | C*T*C*T*T*G*A*A*T*G*G*A*C*C*C*A*G*A*A*G*A*A*A*C*C* |
| Bld26-2 | 174 | C*T*C*T*C*T*T*G*A*A*T*G*G*A*C*C*C*A*G*A*A*G*A*A*A |
| Bld26-3 | 175 | C*A*C*A*C*T*C*T*C*T*T*G*A*A*T*G*G*A*C*C*C*A*G*A*A* |
| Bld26-4 | 176 | T*T*C*A*C*A*C*T*C*T*C*T*T*G*A*A*T*G*G*A*C*C*C*A*G |

^{a}*represents a phosphorothioate linkage, and each sugar of the oligonucleotides is
a 2′-MOE modified sugar.

TABLE 12
Potency

MuSK 67 (IC in nM)

| | IC50 | IC75 |
|---|---|---|
| Bld25-5 | 15.6 | 30.2 |
| Bld25 | 16.5 | 30.4 |
| Bld26 | 17.0 | 20.8 |
| Bld25-4 | 18.0 | n/a |
| Bld25-3 | 20.8 | 34.4 |
| Bld26-2 | 22.8 | n/a |
| Bld26-4 | 28.8 | n/a |

TABLE 13
Selectivity

MuSK 34 (IC in nM)

| | IC50 | IC75 |
|---|---|---|
| Bld26-2 | n/a | n/a |
| Bld25-4 | 49.7 | 52.4 |
| Bld26 | 23.7 | 24.5 |
| Bld26-4 | 19.2 | 69.5 |
| Bld25 | 13.3 | n/a |
| Bld25-5 | 8.4 | 30.4 |
| Bld25-3 | 0.9 | 15.0 |

Table 12 shows the concentration at which there is either 50% (IC50) or 75% (IC75) inhibition in total MuSK67 expression. Table 13 shows the IC10 and IC25 values of MuSK34 (nM) of each ASO, and represents concentration in which there is either 10% (IC10) or 25% (IC25) inhibition in total MuSK expression (MuSK34). ASOs performing exon skipping of exon 6 and/or 7, are expected to show lower IC50/IC75 values of MuSK67 and higher IC10/25 values of MuSK34.

Results from this screen showed that ASOs Bld25, Bld25-5, Bld26-2, and Bld26 showed the best potency and selectivity (see Tables 12 and 13).

### Confirmation of Exon Skipping by Gel Migration

To verify the ASO-induced splicing of the RNA, the genetic sequence from exon 3 to exon 9 was amplified by PCR and the size of the PCR product was measured by migrating it on gel electrophoresis.

Several forms of MuSK RNA exist naturally in human. 3 variants were identified in the literature (FIG. 26):
Variant 1, NM_005592.4 (full length: 972 bp and ΔIg3: 687 bp)
Variant 2, NM_001166280.2 (full length: 783 bp and ΔIg3: 453 bp)
Variant 3, NM_001166281.2 (full length: 708 bp and ΔIg3: 423 bp)

Full length and ΔIg3 indicate the lengths of the full-length sequence and ΔIg3 (6,7) sequence amplified by PCR from exon 3 to exon 9. Compared to variant 1, the exon 8 is missing in variant 2 and variant 3. In addition, a small exon of 30 bases is present only in variant 2. Therefore, skipping exons 6 and 7 would produce 3 additional MuSK RNA forms.

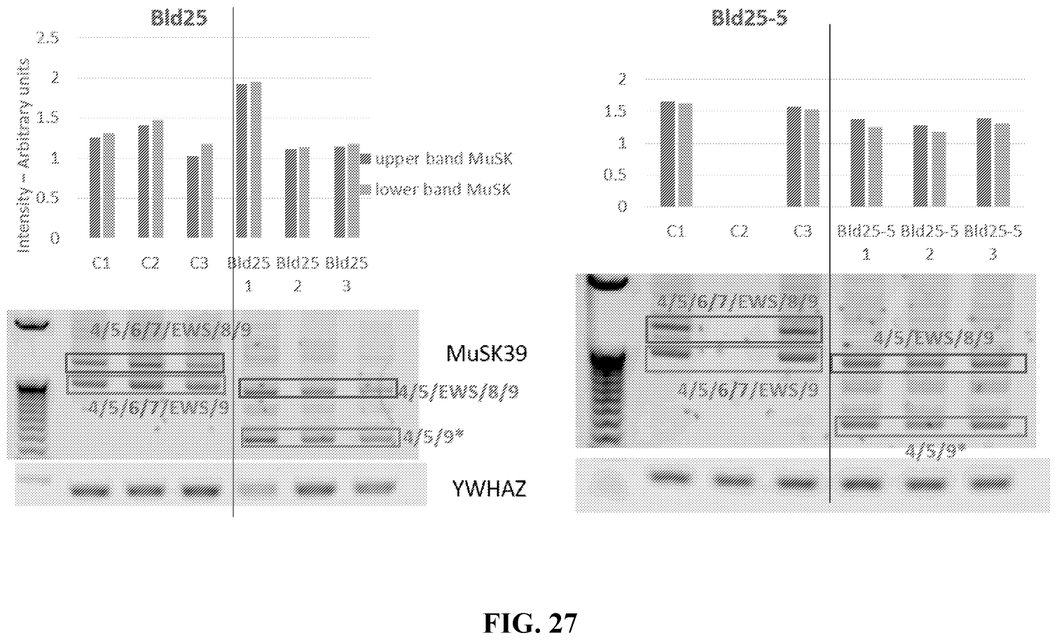

The sequence of exon3-9 of the cDNA from the dose response presented in this Example was amplified by PCR in the conditions without or with Bld25 and Bld25-5 at 50 nM. Results from the gel electrophoresis are shown in FIG. 27. In FIG. 27, “4/5/6/7/EWS/8/9” indicates full length variant 1, “4/5/5+/6/7/EWS/9” indicates full length variant 2, and “4/5/6/7/EWS/9” indicates full length variant 3. Similarly, “4/5/EWS/8/9” indicates ΔIg3 (6,7) in variant 1, “4/5/9” indicates A5+/Ig3 (6,7) of variant 2, and indicates the ΔIg3 (6,7) of variant 3.

As shown in FIG. 27, several bands were detected. In the control conditions, major bands were visible where the full length variants would be expected. In conditions where splicing of the exons 6/7 occurred (confirmed previously by qPCR), major bands were observed at for the spliced versions of the variants.

Intensity of the bands was also examined. Intensity of bands from Bld25 and Bld25-5 products are also shown above the gel images in FIG. 27. These results show that amount of MuSK PCR product with ASO is about 80-90% of the amount of MuSK PCR product in untreated conditions (controls). Therefore, total MuSK is decreased by only a small amount.

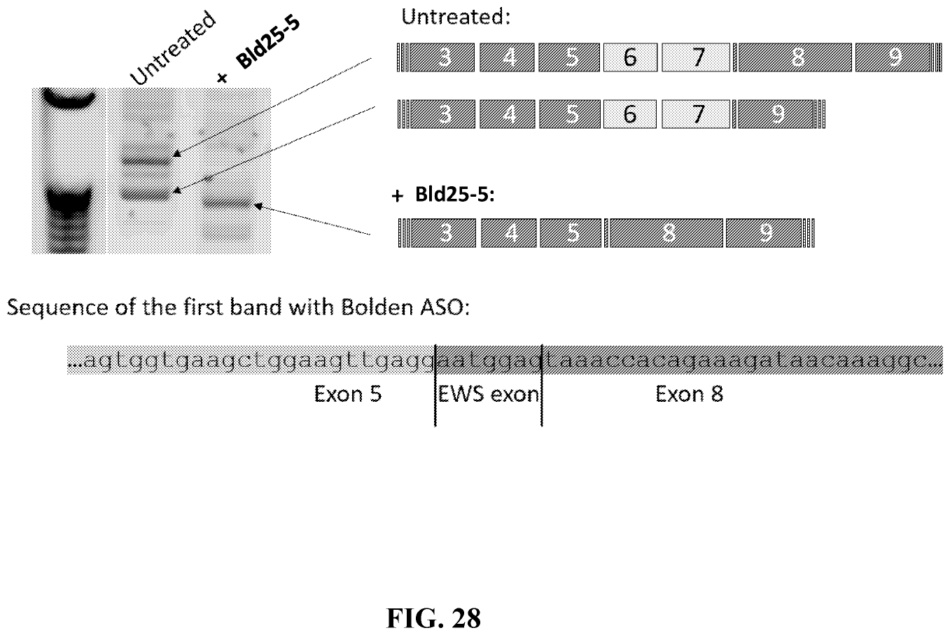

Next, the bands of pcr products from cells treated with ASO 25-5 were sequenced to confirm these bands correspond to the indicated variants. FIG. 28 shows that the sequence of the band lower band positioned where Δ6,7 variant 1 (687 bases) was indeed the sequence of this splice variant.

### Example 7: Design and Screening of Shortened MuSK-Targeting Oligonucleotides Targeting Region 1 of Exon 7

In this Example, additional human MuSK-targeting oligonucleotides were designed by reducing the size of the lead ASOs identified in Example 1 and Example 6 (Bld25, Bld25-5, Bld26, and Bld26-2). These ASOs target region 1 of exon 7 of MuSK SEQ ID NO: 126 (ACTCTGTCAGGTTTCTTCTGGGTCCATTCAAGAGAGTGTGAAAGACCGAGTGATTGACTCA AGAC) which is located at positions 83841-83905 of the MuSK transcript (SEQ ID NO: 77) (or a corresponding regions within other MuSK variant sequences).

Sequences were designed by shortening the sequences of ASOs Bld25, Bld25-5, Bld26- and Bld26-2 by different amounts. ASOs designed in this Example were produced to be 21 and 23 nucleotides in length.

These ASOs were then tested for their ability to induce exon skipping of the MuSK exon 6 and/or exon 7.

### Design of Human ASOs

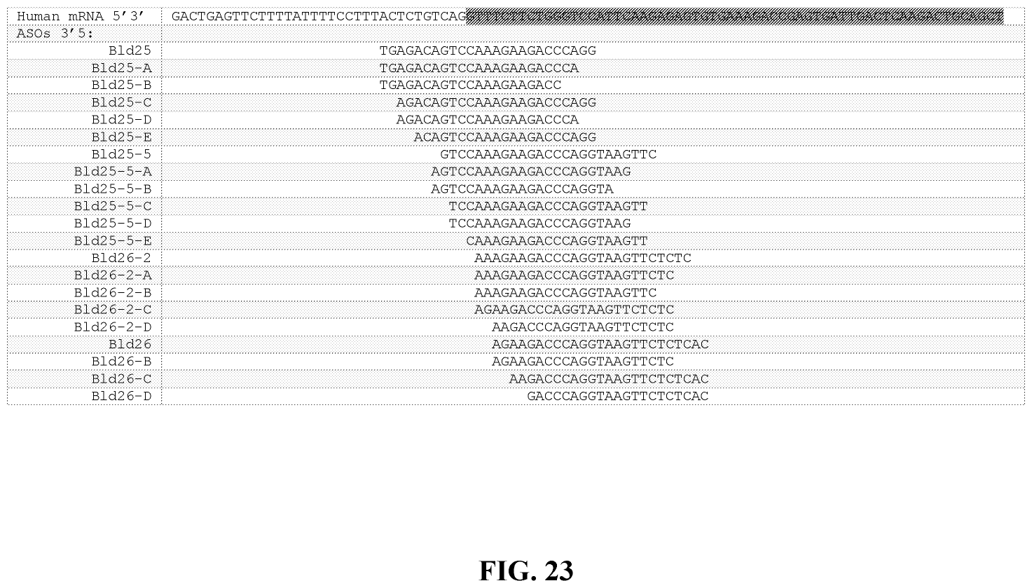

Human antisense oligonucleotides (ASOs) were designed as described above and as shown in FIG. 23. The ASOs were produced by Microsynth (Switzerland). The ASO sequences are shown below in Table 14.

TABLE 14

| Oligo ID | SEQ ID | 5′ to 3′ Sequence |
|---|---|---|
| Bld25-A | 177 | ACCCAGAAGAAACCTGACAGAGT |
| Bld25-B | 178 | CCAGAAGAAACCTGACAGAGT |
| Bld25-C | 179 | GGACCCAGAAGAAACCTGACAGA |
| Bld25-D | 180 | ACCCAGAAGAAACCTGACAGA |
| Bld25-E | 181 | GGACCCAGAAGAAACCTGACA |
| Bld25-5-A | 182 | GAATGGACCCAGAAGAAACCTGA |
| Bld25-5-B | 183 | ATGGACCCAGAAGAAACCTGA |
| Bld25-5-C | 184 | TTGAATGGACCCAGAAGAAACCT |
| Bld25-5-D | 185 | GAATGGACCCAGAAGAAACCT |
| Bld25-5-E | 186 | TTGAATGGACCCAGAAGAAAC |
| Bld26-2-A | 187 | CTCTTGAATGGACCCAGAAGAAA |
| Bld26-2-B | 188 | CTTGAATGGACCCAGAAGAAA |
| Bld26-2-C | 189 | CTCTCTTGAATGGACCCAGAAGA |
| Bld26-2-D | 190 | CTCTCTTGAATGGACCCAGAA |
| Bld26-B | 191 | CTCTTGAATGGACCCAGAAGA |
| Bld26-C | 192 | CACTCTCTTGAATGGACCCAGAA |
| Bld26-D | 193 | CACTCTCTTGAATGGACCCAG |

All ASOs in Table 14 were designed to include 2′-MOE modification on each sugar and where there is a phosphorothioate internucleotidic linkage between each nucleotide. All ASOs in Table 14 were 21 or 23 nucleotides in length.

### Screen of 21-Mer and 23-Mer Antisense Oligonucleotides Targeting Exon7 Region 1

ASO transfection and cell culture, RNA extraction, cDNA transcription, qPCR were performed according to the methods described in Example 1, except as where indicated otherwise.

i. Alignment to the Genetic Sequence

The alignment of the ASOs in Table 14 on the genetic sequence of Exon7 of MuSK is presented in FIG. 23.

ii. Cell Observations

LHCN-M2 cells were plated at 35,000 cells/well in gelatin pre-coated 6-well plates and were transfected 48 h after seeding with ASO at 5 nM, 12.5 nM, 25 nM, 50 nM, and 125 nM. Each of the ASOs in Table 14 were tested in addition to Bld25, Bld25-5, Bld26, and Bld26-2 (leads identified in Examples 1 and 6). 24 h after transfection, cells were observed (data not shown) and the RNA was extracted and analyzed.

After 24 h of transfection, cells looked healthy in all conditions (data not shown).

iii. MuSK Expression Analyses

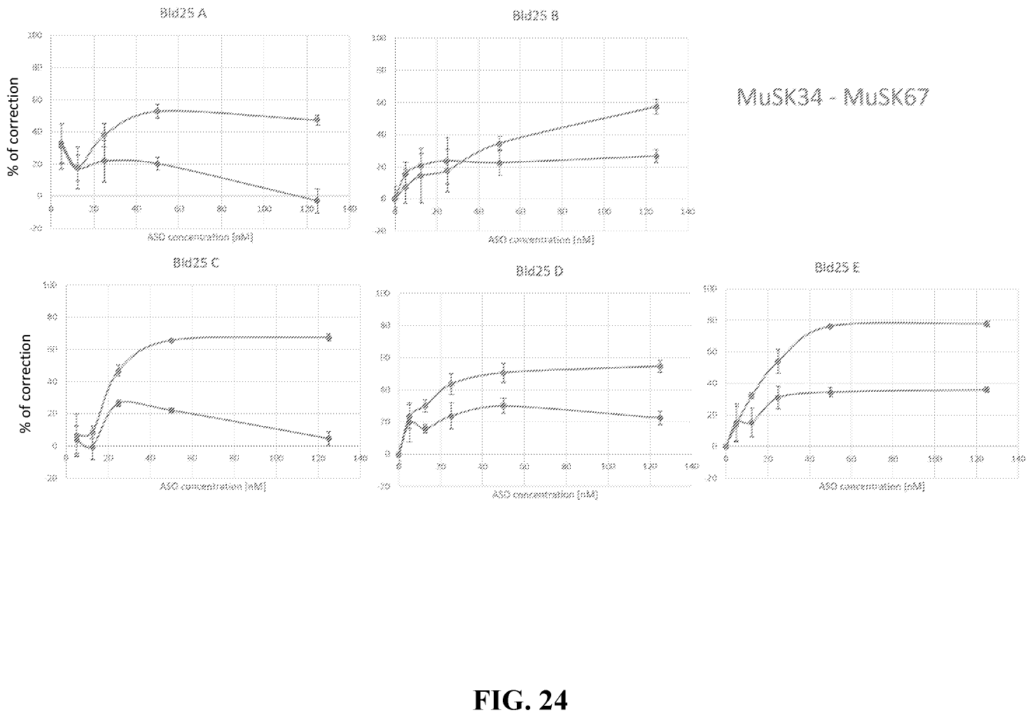

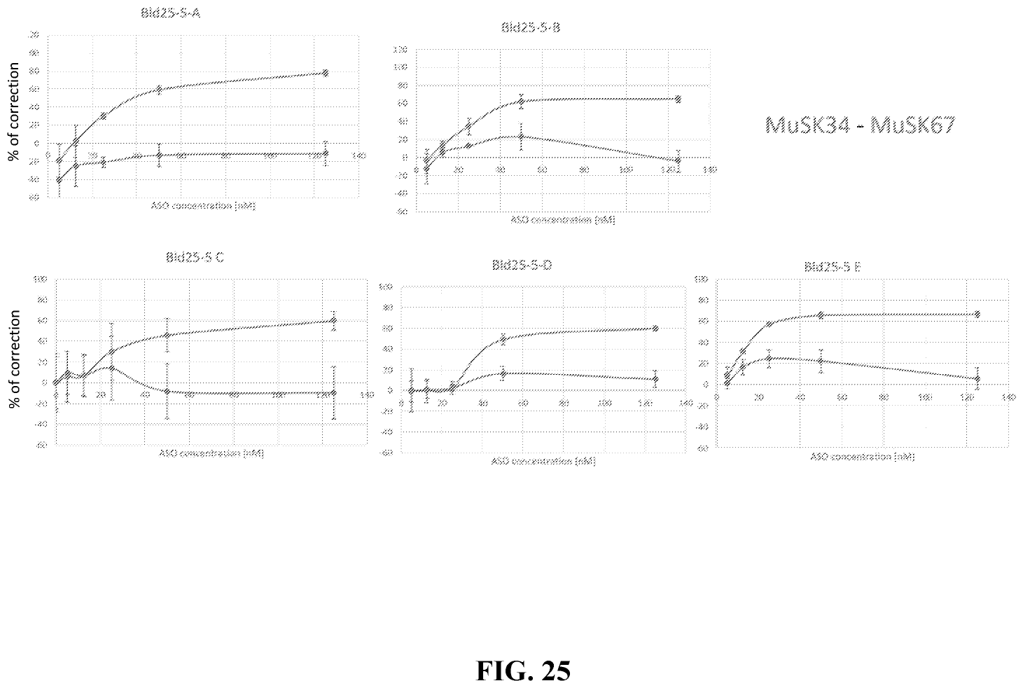

24 h after transfection, RNA was extracted, and MuSK gene expression was analyzed in qPCR (FIGS. 24-25).

ASOs with the modifications used in this Example are shown in Table 15 below.

TABLE 15

| Oligo ID | SEQ ID NO: | Sequence-(5′ to 3′)^{a} |
|---|---|---|
| Bld25-A | 194 | A*C*C*C*A*G*A*A*G*A*A*A*C*C*T*G*A*C*A*G*A*G*T |
| Bld25-B | 195 | C*C*A*G*A*A*G*A*A*A*C*C*T*G*A*C*A*G*A*G*T |
| Bld25-C | 196 | G*G*A*C*C*C*A*G*A*A*G*A*A*A*C*C*T*G*A*C*A*G*A |
| Bld25-D | 197 | A*C*C*C*A*G*A*A*G*A*A*A*C*C*T*G*A*C*A*G*A |
| Bld25-E | 198 | G*G*A*C*C*C*A*G*A*A*G*A*A*A*C*C*T*G*A*C*A |
| Bld25-5-A | 199 | G*A*A*T*G*G*A*C*C*C*A*G*A*A*G*A*A*A*C*C*T*G*A |
| Bld25-5-B | 200 | A*T*G*G*A*C*C*C*A*G*A*A*G*A*A*A*C*C*T*G*A |
| Bld25-5-C | 201 | T*T*G*A*A*T*G*G*A*C*C*C*A*G*A*A*G*A*A*A*C*C*T |
| Bld25-5-D | 202 | G*A*A*T*G*G*A*C*C*C*A*G*A*A*G*A*A*A*C*C*T |
| Bld25-5-E | 203 | T*T*G*A*A*T*G*G*A*C*C*C*A*G*A*A*G*A*A*A*C |
| Bld26-2-A | 204 | C*T*C*T*T*G*A*A*T*G*G*A*C*C*C*A*G*A*A*G*A*A*A |
| Bld26-2-B | 205 | C*T*T*G*A*A*T*G*G*A*C*C*C*A*G*A*A*G*A*A*A |
| Bld26-2-C | 206 | C*T*C*T*C*T*T*G*A*A*T*G*G*A*C*C*C*A*G*A*A*G*A |
| Bld26-2-D | 207 | C*T*C*T*C*T*T*G*A*A*T*G*G*A*C*C*C*A*G*A*A |
| Bld26-B | 208 | C*T*C*T*T*G*A*A*T*G*G*A*C*C*C*A*G*A*A*G*A |
| Bld26-C | 209 | C*A*C*T*C*T*C*T*T*G*A*A*T*G*G*A*C*C*C*A*G*A*A |
| Bld26-D | 210 | C*A*C*T*C*T*C*T*T*G*A*A*T*G*G*A*C*C*C*A*G |

^{a}*represents a phosphorothioate linkage, and each sugar of the oligonucleotides is
a 2′-MOE modified sugar.

Results from this screen showed that ASOs Bld25-E and Bld25-5-A showed the best activity (see FIG. 24 and FIG. 25). Bld25-E showed MuSK67 maximum correction of 80%, with less of an effect on total MuSK (MuSK34), and an IC50 of MuSK67 of about 21 nM. Bld25-5-A showed MuSK67 maximum correction of 80%, with less of an effect on total MuSK (MuSK34) and an IC50 of MuSK67 about 35 nM. These results indicate that ASOs Bld25-E and Bld25-5-A are additional candidates to effectuate exon6-7 skipping in MuSK and have shorter lengths (21-mer and 23-mer) than the lead ASOs identified from Examples 1 and 6.

### Example 8: Analysis of MuSK Protein

This Example aims to examine the resulting MuSK protein after treatments with the ASOs described herein.

### Timing of Effect on MuSK Protein

In order to examine resulting MuSK protein produced from ASO-treated cells, it is important to understand the relative timing of the effect of targeting MuSK with RNA.

Length of time from RNA treatment was examined to determine how long after treatment will there be an effect on MuSK protein (i.e., production of MuSKΔIg3).

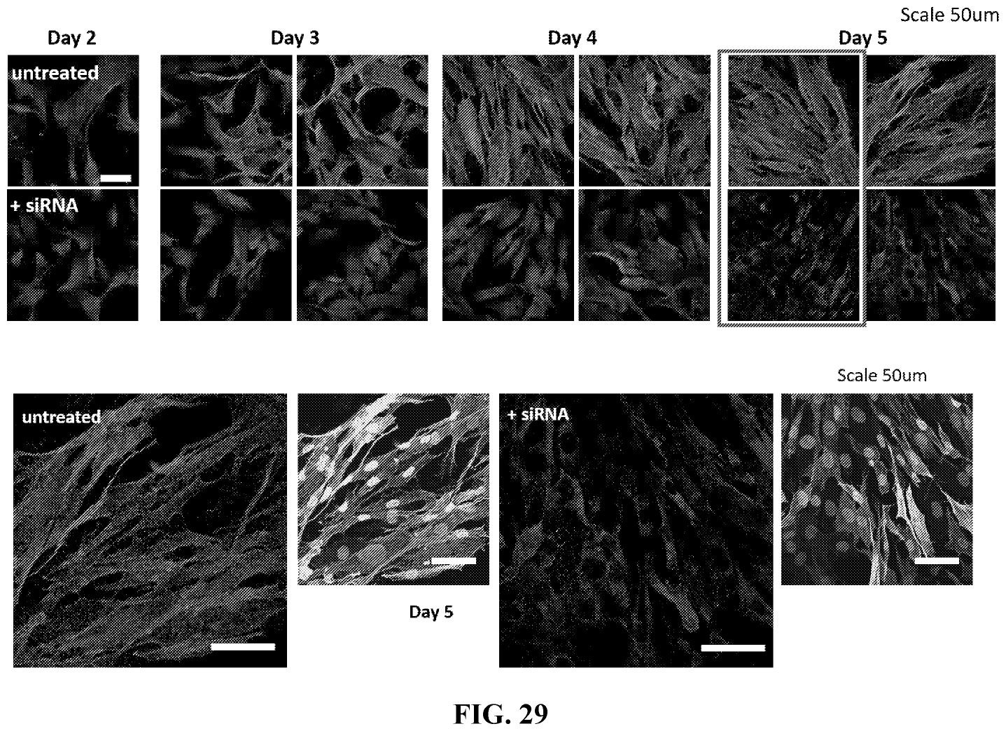

In this experiment, LHCN-M2 cells were treated for 48 h with 10 nM of siRNA (Thermofisher, 4392420, assay ID s224071) against MuSK and were then stained for MuSK protein on days 2, 3, 4, and 5 (with MuSK antibodies isolated from patients with a disease involving the natural production of MuSK antibodies followed by goat anti-human (A-2133)) following siRNA treatment. Results for each timepoint in treated and untreated cells are shown in FIG. 29. Results show that in the untreated wells, MuSK protein amount increases while cells start to be confluent. In wells treated with siRNA, MuSK protein amount decreases, especially 5 days after the transfection. Overall, these results show that MuSK protein amount can be modulated, and the best timing to observe an effect is 5 days after modulating the RNA.

FIG. 29 shows the cells at Day 5 magnified and stained for MuSK (red) (with MuSK antibodies isolated from patients with a disease involving the natural production of MuSK antibodies followed by goat anti-human (A-2133)) and actin phalloidin (green) (ab ab176743 at 1:1000). These results confirm that (i) the MuSK antibody used to stain for MuSK protein works in immunofluorescence, (ii) MuSK protein level increases over time, probably along with cell differentiation, and (iii) MuSK siRNA (“Si-MuSK”) induces a reduction of MuSK protein, especially after 5 days of exposure.

### Measuring Removal of Ig3 Domain of MuSK

Since ASOs as described herein are targeting the Ig3 domain and inducing exon skipping (6,7) to produce MuSKΔIg3, there needs to be a way to detect MuSKΔIg3 protein. Timing of the effects of the ASOs/RNA on MuSK protein has been established. An assessment (e.g., Western blot analysis) will be performed. Such an assessment includes assessment of MuSKΔIg3 protein produced from cells treated with ASOs described herein. A MuSK antibody that targets the MuSK cytoplasmic domain will be utilized for identification of MuSKΔIg3 protein. Antibodies are produced and tested using the recombinant MuSK cytoplasmic domain.

Since human and murine MuSK protein sequences vary, human MuSK controls should be used for assessment (e.g., by Western Blot). Positive controls (i.e., MuSKΔIg3 protein and human FL MuSK protein) are used to confirm the forms of MuSK that are present. Plasmids coding for the different WT and D3 (Ig3) isoforms of MuSK protein are obtained. Cells are transfected with the plasmids and positive control isoforms are expressed and obtained.

Human MuSK antibodies targeting the cytoplasmic domain of MuSK and human MuSK positive controls (i.e., MuSKΔIg3 protein and human FL MuSK protein) are obtained, and such reagents are used in a assessment (e.g., Western Blot) to characterize the MuSK protein produced from cells treated with ASOs described herein.

I. LISTING OF SEQUENCES

Oligonucleotide (SEQ ID NO: 1)
GCTAGGGTGGTCTTTTAGAAATGCA
Oligonucleotide (SEQ ID NO: 2)
GGTCAAGCTAGGGTGGTCTTTTAGA
Oligonucleotide (SEQ ID NO: 3)
CTGCAGGAAATGGTCAAGCTAGGGT
Oligonucleotide (SEQ ID NO: 4)
GAAGTGGTGAGTGACGCTCCTGCAG
Oligonucleotide (SEQ ID NO: 5)
GTTAGGAAGACAGAAGTGGTGAGTG
Oligonucleotide (SEQ ID NO: 6)
ATCCTGGCAAAAACTGTTAGGAAGA
Oligonucleotide (SEQ ID NO: 7)
GTGGGATTCAGGAGCCCGCAGGATC
Oligonucleotide (SEQ ID NO: 8)
GGTGACATTGTGGGATTCAGGAGCC
Oligonucleotide (SEQ ID NO: 9)
GGAGCCAAAGGTGACATTGTGGGAT
Oligonucleotide (SEQ ID NO: 10)
GGTCACAAAGGAGCCAAAGGTGACA
Oligonucleotide (SEQ ID NO: 11)
ACAGTGCAGGGTCACAAAGGAGCCA
Oligonucleotide (SEQ ID NO: 12)
CTGTTGCTGTACAGTGCAGGGTCAC
Oligonucleotide (SEQ ID NO: 13)
GGGACAGGAATGCCTGTTGCTGTAC
Oligonucleotide (SEQ ID NO: 14)
CAGGTGATGGTGGGGACAGGAATGC
Oligonucleotide (SEQ ID NO: 15)
CCGTTTTCAATCCAGGTGATGGTGG
Oligonucleotide (SEQ ID NO: 16)
TGACACTCACAGCATTTCCGTTTTC
Oligonucleotide (SEQ ID NO: 17)
AAGTCCCCACACACATGACACTCAC
Oligonucleotide (SEQ ID NO: 18)
GGTCTTCCCCAGACAAGTCCCCACA
Oligonucleotide (SEQ ID NO: 19)
ACTATGTCAGTAGATTTGAAGGGAA
Oligonucleotide (SEQ ID NO: 20)
TCCCACTATACTATGTCAGTAGATT
Oligonucleotide (SEQ ID NO: 21)
TCAGTCAAGGATTTCCCACTATACT
Oligonucleotide (SEQ ID NO: 22)
AAAAGAACTCAGTCAAGGATTTCCC
Oligonucleotide (SEQ ID NO: 23)
GTAAAGGAAAATAAAAGAACTCAGT
Oligonucleotide (SEQ ID NO: 24)
AACCTGACAGAGTAAAGGAAAATAA
Oligonucleotide (SEQ ID NO: 25)
GGACCCAGAAGAAACCTGACAGAGT
Oligonucleotide (SEQ ID NO: 26)
CACTCTCTTGAATGGACCCAGAAGA
Oligonucleotide (SEQ ID NO: 27)
CACTCGGTCTTTCACACTCTCTTGA
Oligonucleotide (SEQ ID NO: 28)
GTCTTGAGTCAATCACTCGGTCTTT
Oligonucleotide (SEQ ID NO: 29)
GATAAACAGCTGCAGTCTTGAGTCA
Oligonucleotide (SEQ ID NO: 30)
AGTCCTGGCTTGGTGATAAACAGCT
Oligonucleotide (SEQ ID NO: 31)
ATGTGTAGAGTCCTGGCTTGGTGAT
Oligonucleotide (SEQ ID NO: 32)
GTAGCTATGCATGTGTAGAGTCCTG
Oligonucleotide (SEQ ID NO: 33)
TGCTTATTGGTAGCTATGCATGTGT
Oligonucleotide (SEQ ID NO: 34)
ACTTCTCCCCATGCTTATTGGTAGC
Oligonucleotide (SEQ ID NO: 35)
CCTTGGCAGTACTGAACTTCTCCCC
Oligonucleotide (SEQ ID NO: 36)
CCTGCTATGCTGATGGTGGCTGCAG
Oligonucleotide (SEQ ID NO: 37)
GGGCATCCTACCTGCTATGCTGATG
Oligonucleotide (SEQ ID NO: 38)
GCAAATGTGAAGGGGCATCCTACCT
hu6-75; SEQ ID NO: 39
TGCATTTCTAAAAGACCACCCTAGC
hu6-69; SEQ ID NO: 40
TCTAAAAGACCACCCTAGCTTGACC
hu6-58; SEQ ID NO: 41
ACCCTAGCTTGACCATTTCCTGCAG
hu6-39; SEQ ID NO: 42
CTGCAGGAGCGTCACTCACCACTTC
hu6-27; SEQ ID NO: 43
CACTCACCACTTCTGTCTTCCTAAC
hu6-12; SEQ ID NO: 44
TCTTCCTAACAGTTTTTGCCAGGAT
hu610; SEQ ID NO: 45
GATCCTGCGGGCTCCTGAATCCCAC
hu619; SEQ ID NO: 46
GGCTCCTGAATCCCACAATGTCACC
hu628; SEQ ID NO: 47
ATCCCACAATGTCACCTTTGGCTCC
hu637; SEQ ID NO: 48
TGTCACCTTTGGCTCCTTTGTGACC
hu646; SEQ ID NO: 49
TGGCTCCTTTGTGACCCTGCACTGT
hu656; SEQ ID NO: 50
GTGACCCTGCACTGTACAGCAACAG
hu669; SEQ ID NO: 51
GTACAGCAACAGGCATTCCTGTCCC
hu681; SEQ ID NO: 52
GCATTCCTGTCCCCACCATCACCTG
hu693; SEQ ID NO: 53
CCACCATCACCTGGATTGAAAACGG
hu6110; SEQ ID NO: 54
GAAAACGGAAATGCTGTGAGTGTCA
hu6125; SEQ ID NO: 55
GTGAGTGTCATGTGTGTGGGGACTT
hu6139; SEQ ID NO: 56
TGTGGGGACTTGTCTGGGGAAGACC
hu7-75; SEQ ID NO: 57
TTCCCTTCAAATCTACTGACATAGT
hu7-66; SEQ ID NO: 58
AATCTACTGACATAGTATAGTGGGA
hu7-53; SEQ ID NO: 59
AGTATAGTGGGAAATCCTTGACTGA
hu7-45; SEQ ID NO: 60
GGGAAATCCTTGACTGAGTTCTTTT
hu7-33; SEQ ID NO: 61
ACTGAGTTCTTTTATTTTCCTTTAC
hu7-22; SEQ ID NO: 62
TTATTTTCCTTTACTCTGTCAGGTT
hu7-10; SEQ ID NO: 63
ACTCTGTCAGGTTTCTTCTGGGTCC
hu73; SEQ ID NO: 64
TCTTCTGGGTCCATTCAAGAGAGTG
hu717; SEQ ID NO: 65
TCAAGAGAGTGTGAAAGACCGAGTG
hu730; SEQ ID NO: 66
AAAGACCGAGTGATTGACTCAAGAC
hu744; SEQ ID NO: 67
TGACTCAAGACTGCAGCTGTTTATC
hu758 ; SEQ ID NO: 68
AGCTGTTTATCACCAAGCCAGGACT
hu766; SEQ ID NO: 69
ATCACCAAGCCAGGACTCTACACAT
hu776; SEQ ID NO: 70
CAGGACTCTACACATGCATAGCTAC
hu785; SEQ ID NO: 71
ACACATGCATAGCTACCAATAAGCA
hu796; SEQ ID NO: 72
GCTACCAATAAGCATGGGGAGAAGT
hu7111; SEQ ID NO: 73
GGGGAGAAGTTCAGTACTGCCAAGG
hu7136; SEQ ID NO: 74
CTGCAGCCACCATCAGCATAGCAGG
hu7146; SEQ ID NO: 75
CATCAGCATAGCAGGTAGGATGCCC
hu7158; SEQ ID NO: 76
AGGTAGGATGCCCCTTCACATTTGC
MuSK Genomic Sequence (SEQ ID NO: 77)

| | |
|---|---|
| 1 | tatacagtca tttatcactt aacatcaggg atatgttctg agaaatgcat ccatagttca |
| 61 | ttttgttatt gtgtgaacat catagagtat acttacagaa accttgatgg tatagcccac |
| 121 | tgcacaccta ggctgtatgg atagcctatt gcccgtaggc tacaaacctg tcagcatgtt |
| 181 | actacactga atactgtagg caattgtagc acaatggtac gtatttgtgt atctaaacat |
| 241 | agaaaacata cagtaaaaat acagtataaa agataaaaat ggtacacctg cattgggcac |
| 301 | ttaccatgaa cgaagatttc agcactgaag tttgctctgg gtaagtcagt gagtgagtga |
| 361 | tgtgtgaatg tgaaggccta ggacattact gtacactgct gtaaacttta taaatactgt |
| 421 | acacttagac tacgctaaat ttatttttaa aatatttttc tttctttaat aataaattaa |
| 481 | ccttagctta ccacaactat tttattttat aaacttttaa aatttgtaat ctttttgact |
| 541 | cttttgtaat aatactctgt ttgaaacaca aatatattgt acagttatac aaaaatagtt |
| 601 | tttcttcata tccttaagct cttttctatc ttaaaatttt atttttattt tttactttct |
| 661 | aaactttttt tgttaaacac taagacaaaa acacacaata gccttggcct acacagggtc |
| 721 | aggatcaaaa catcactgcc tttcaacagc ttgtcctact ggaaggtcct caggggcaat |
| 781 | aacacacatg gagctgccat ctcctatggt aacaatgtat tcttctagaa tacctgcctg |
| 841 | aggctgtttt acaatcaatt ttttatttat ttttattttt tatgttttta aataagtaca |
| 901 | gggtatacac agtaaaataa taataaaaat atagtacatt aaaaaaacca gcaacatggt |
| 961 | catttattat cattatcaac tattatgtac tctacataat tgtatgtgct tttttattca |
| 1021 | aaaagaaaat cattaagaac acagcaatat taatatttat tctttttttt agaagaacct |
| 1081 | gatttattta tttaattttt ttattatact ttaagttctg ggatacatgt gcaaaacgtg |
| 1141 | taggtttgtt acacaggtat acatgtgcca tggtggtttg ctgtatccat caacccatca |
| 1201 | tctacattag gcatttctcc taatgctatc cctcccctag ccccccaccc cttgacaggc |
| 1261 | cccagtatgt gattctcccc tccctgtgtc catgtgttct cattgttagc tcccacttat |
| 1321 | gagtgagaac atgtggtgtt tggttttctg ttcctgtatt agtttgctga gaatgatggt |
| 1381 | ttccagcttc atccatgtcc ctgcaaagga tataaactca ttgtttttta tggctgcata |
| 1441 | gtattccgta gtgtatatgt gccacatttt ctttatctag tctatcattg attggcattt |
| 1501 | gggttggttc caagtctttg ccattgtgaa tagtgctgaa ataaacatat atgtgcatgt |
| 1561 | gtctttatag tagaatgatt tatagtcctt tgggtatata cccagtaatg agattgctgg |
| 1621 | gtcaaatggt atttctggtt ctaatccttg aagaattgcc acactgtctc ccacaatggt |
| 1681 | tgaactaatt tacactccca caaacagtgt aaaagcattc ctatttttcc acattctctc |
| 1741 | cagcatcggt tgcttcctga ctttttaatg attgccactc taacaggcat gagatggtat |
| 1801 | ctcattgtgg ttttgatttg catttctcta atgaccaata atgatgagct ttatttcata |
| 1861 | tgtttgttgg ctgcataaat gtcttatttt gagaagtgtc tgttcatatc cttcgcccac |
| 1921 | tttttgatgg ggttgtttgt ttttttcttg taaatttgtt taagttcttt gtagattctg |
| 1981 | gatattagcc ctttgtcaga tggatagatt gcaaaaattt tctcccactc tgtaggttgc |
| 2041 | ctgctcactc taatgatagt tccttttgct gagcagatgg tctttagttt aattaggtcc |
| 2101 | catttgtcaa ttttgacttt tgttgccatt gcttttgggg ttttagtcat gaagtctttg |
| 2161 | cccatgccta tgtcctgaat ggtattgcct aggttttctt ctagggtttt tatggtttta |
| 2221 | ggttttacat ttaagtattt aatccatctt gagttaattt ttgtatgaga tataaggaag |
| 2281 | gggtccagtt ccagttttcc gcatatggct agccagtttt cccaacacca tttattaaat |
| 2341 | agggaatcct ttccctactg cttgtttttg tcaggtttgt caaagatcag atggttgtag |
| 2401 | atgtgtggca ttatttctta ggcctctgtt ctgttccatt ggtctatata tctgttttgg |
| 2461 | taccagtacc atgctgtttt ggcaaaccag gaagaagctg aatccctgaa tataccaata |
| 2521 | agaagttctg aaattgaggc agtaattaat agcctaccga gcaaaaaaaa aaaaaaaaaa |
| 2581 | aaaaaaatcc aggaccagac ggattcacag ccgaattcta ccagaggtac aaagaggagc |
| 2641 | tggtaccatt ccttctgaaa ctattccaat caatagaaaa agagggaatc ctctctaact |
| 2701 | aattttatga gtccagcatc atcctgatac caaaacctgg cagaggcaca accaaaaaca |
| 2761 | gaaaatttca ggccaatatc cctgatgaat attgatgtga aaattctcaa taaaatactg |
| 2821 | gcaaactgaa tccaacagca catcaaaaag cttacccacc acgatcaagt tggcttcatc |
| 2881 | cctgggatgc aaggctggtt aaacatatgc aaatcaataa atgtaatcca tcacataaac |
| 2941 | agaaccagtg acaaaaacca catgattatc tcaatagatg cagaaaaggc tttcaataaa |
| 3001 | atttaacacc cctttatgct aaaaactctc aataaactag gtattgatgg aacccatcga |
| 3061 | aaaaaaatga gctatttatg acaaacccac agccaatgtc acactgaatg agaaaaagct |
| 3121 | ggaagtattc cctttgaaaa ctggcacgag acaaggatgc cctctctcac cactcgtatt |
| 3181 | caacctacta ttggaagttc tggccagggc aatcaggcaa gagaaagaaa taaagcgtat |
| 3241 | tcaaatagga agagagaaag tcaaattgtc tctgtttgca gatgacatga ttgtatattt |
| 3301 | agaaaacccc atcatctcag ccccaaatct ccttaagctg ataagcaact tcagcaaagt |
| 3361 | cccaggatac aaaatcaatg tgcaaagatc acaagattcc tatataccaa taacagacaa |
| 3421 | acagagagcc aaatcatgag tgaactccca ttcacaattg ctacaaagag aataaaatac |
| 3481 | ctaggaatcc aacttacaag ggatatgaag gacctcttca aggagaacta caaaccactg |
| 3541 | ctcaagggaa taaaagagga cacaaataaa tggaaaaaca ttccatgctc atgataggaa |
| 3601 | gaatcaatat tgtgaaaatg gctgtactgc ccaaggtaat ttatagattc aatgctatcc |
| 3661 | ccatcaagct accattgact ttcttcacag aattagaaaa aactacttta aatttcatat |
| 3721 | ggaaccaaaa aagagcccat atagccaagg caatcctaag caaaaagaac aaagctggcg |
| 3781 | gcatcacact acctgactct aaagttctat aaatgaactc acgaggccaa agaatgaaga |
| 3841 | agacaccaac acaaagggga aaaaatcaaa acaatatggt cattgcaatg gtagagatat |
| 3901 | gcacagtata ttatggaaac ccagagaaga caaacgtaac acagctttgg gaagatatgg |
| 3961 | aatttttctc ccaggaggag aggatgatca agctgagaga tgtgtgaact gagtggaaag |
| 4021 | gaggggagta gtattccaag agaagcaaac agtatgagca attcatggag gtaggaaagc |
| 4081 | tgtatacagg gaaccatcag caggttgttg aataaaggaa gctgaatgag gctgggcaca |
| 4141 | gtagctcacg cctgcattcc cagcactttg agaggccgag gtggaatcat catttgaggc |
| 4201 | cagcagtttg agaccagcct gggtaacata gcctgaccac atctctccaa aaaataaaaa |
| 4261 | taaattagcc tgctgtagtg gcagtgcctg tagtcctagc tactctgaaa ggatcacttg |
| 4321 | agtccaggag gctgaggttg cagtgagcta tgattacccc attgcactcc agcctgggca |
| 4381 | acagagcaag accctgactc tgaaaaaaaa aaaaaacaaa aaacaaaaac aggatcgtaa |
| 4441 | aagacaatac taacatacaa caaagcatgc agattccagt aaagcctgtc atattaggca |
| 4501 | gattaggcag gtttaaatat atttttatgt gatgatgagc ccctaaaggc ttttgagcag |
| 4561 | gagggactat ctgaaaaaag atatagaatg gttaagaccc aagacagaga gaaggtacaa |
| 4621 | ggactatttg gttgataaaa tctgtaggaa ttggaatggg ctgaatgtgg gagtgggggt |
| 4681 | ggggtgagag atggggagga ctgaagactg aatagaccca gatttctggt ttgagtccgg |
| 4741 | gagaggtggg gtgctgaatt cgaaggtcag gacacctata cctctgggaa tacagcgatg |
| 4801 | gttgcaaaag gcatctggta ttgtccttct gccactgtct taaaagggat tctaaaggga |
| 4861 | ttttcttgcc cttgttcctc cagccctaca cttgcattac tggcctacgt ggatgctcac |
| 4921 | cagctggctg tgtccatcat tgcagccaag tctgagaatt cctgattata cttagagcct |
| 4981 | gtggagctgc tgacacaaac agtcattagc agacaaccct tttgcaacaa agtatgcttt |
| 5041 | aaaatgtaaa ctgtggagcc attttccttg cgttgtccag aaggaacttc gtcctgcgtg |
| 5101 | agcctggatt aatcatgaga gagctcgtca acattccact ggtacatatt cttactctgg |
| 5161 | ttgccttcag cggaactgag aaacttccaa aaggttggtt tgagcaatcg tgtcttcttg |
| 5221 | ttgtcttgtc atggttgtaa agtgtgtgta tatgagatat gaccaagact gatttatagt |
| 5281 | atggtgggct tgggttttac ttgaagaagt aagggtgaaa tgtatgatga gggaggaaat |
| 5341 | tatatacata agcaattggt tgatggtctc tctaccaata ttttactccg aagttctgca |
| 5401 | ataaaaggct tagccacata gtgcatgcag agctctgact tctagggcaa aacataagca |
| 5461 | tgtacactgc aagttatcaa ggtctatttt gtttatgcga atgtcaaatt tcagggtggt |
| 5521 | ctaagtggat gatgacaacc ttttcgcatc aggtttgcca ggaatagaat caaccccatt |
| 5581 | ctcagacatc cagagcattt gcttctgaat aaaaatcact tttattttca gttttagctt |
| 5641 | tgccaaactc ttaataaata ttttagttgc taaaaattgg aaatacagaa agacgtacac |
| 5701 | aatttgcctg tgtaacaaac ccggaagaca atggatttta aataactttg aggctggagt |
| 5761 | catgatttgg cttggcaaga gactgcaaaa gtttttttca gagcctttgg aatgtaccgg |
| 5821 | aaataacacc acctcagggg aaaggaaagc aacttaagaa actgacaaac acctgttgca |
| 5881 | gtagcaagaa tgtccagtga tatagtttct ggccagtgta ttatgaaact gggactagcg |
| 5941 | tatttgaaat cagaaagact ggaaagtctg tgatttgcaa agtgactaag ggtatcactg |
| 6001 | aaatcttttt ttaaaaagac atgagaaagc agatcgagtg gtatcaatac aagagttttc |
| 6061 | aaatttaatg ttataccata gagtaaaaag agatttttgc ttcaggacat gttcaaatta |
| 6121 | atcattttcc ctttatatta atcaaagcac tgaaacagtt tgtattgtgt agataaatga |
| 6181 | aggtggtatc tgtatataga tgcaaaatgt atctttagat tttctggttc actaaaaatt |
| 6241 | ctggagccat aaggtcaaag ttcacatgca aataagatgc gggtttcttc acaaacaatc |
| 6301 | tgatttcaca gatttttttg tttcattttg ttgcttctct ttttccattt tacttgagta |
| 6361 | taatggtatc ttcaaatgta tttaaacatt taatttctaa tgaatacttt aaggcatcta |
| 6421 | cagataaaag agactctaga tcttttcaaa cagctaataa tagtcagaaa gacttaacat |
| 6481 | tggcttcaga ttattttaat acttcctgat caaacaacgg atgtccggta aggccccacc |
| 6541 | tggatgtttg aacgtatggg ttgtcttaag tattatttgt atatttctaa accacgcagt |
| 6601 | tacataaaaa tgatagggtt aaatactaaa atacagtctt ataaagagca ggacaattga |
| 6661 | ttttgatgat tcaaataagg atttgttgta cacacacaca aaacccccaa aatctctcta |
| 6721 | tctgaataag tcttaattta tattctagaa agaattgatt ttcatttcca aactttacaa |
| 6781 | ataaagccct taattcaaat gcacactatc tctattttga aaatattgaa atatagtggc |
| 6841 | atagagataa caaagccaaa caccctgact tttaaataaa ctaaccagat cattcaccca |
| 6901 | atttcatttc ttataattta tttactacta gaattaggtt ttaaaatcaa aaaatttaag |
| 6961 | caattttttc tcagtctatt gtactgtaaa atctaaatta gtatgagttt aacatcactc |
| 7021 | ttagccaaaa taccatgtct aggagaacaa acatttattt actacataca gcagttctga |
| 7081 | gaataactaa tatcatgaaa gcataatcct taaaatttta aatgttgaag ggtgaggaat |
| 7141 | acattggtag aaaagaacca caataaaatt ttattatttt cattattatt attattattt |
| 7201 | gagacagagt ctcgttctgt tgccaggctg cagtgcagtg gtgtgatctc agctcactgc |
| 7261 | aacctcctcc tcccaggttc aagctatact cctgcctcag actcgcaaat acctgggact |
| 7321 | acaggtgcaa gccaccacac tcggctaatt tttatatttt tagtagagac agggtttcac |
| 7381 | catgttggcc aagatggtct ccatctcttg acctcgtgat ctgcctgcct cggcctccca |
| 7441 | aagtgctggg attacaggca tgagccactg cgccagccat tatactactg aaactaataa |
| 7501 | aaatggccca cccaaactat ttctactatt tctagaaaac tactgttttc cctattcttg |
| 7561 | gcagacggta ataacaaaaa gagtttttta gataatatac ttcttcctaa gtcctttatc |
| 7621 | atgaatgcat aaaggaaatt gtacaatatg tattagctaa taaattgaca tatttaaaat |
| 7681 | aacacaaaag tattcagtgg attattttct gactttctgc acatcatttt ttgataaaaa |
| 7741 | ttaatatcca actttgtaga attaattgtt catagttttc attttagttt aattaaggct |
| 7801 | actaatgccc aatgataagg ggaagaatca ttgtctttct gaatcatttc aactagtgat |
| 7861 | attttcaatg atgaattcag tgccataacc caagtagtag aatttcacaa actgtcattt |
| 7921 | atgaaagtag ccaatgttcc actggataat tggcttaact acatacatca ataacattta |
| 7981 | atgctttatt agggagtatt aaatgtatgg tgcaatgtta gattctgcag gagcataaga |
| 8041 | gttcatgtaa gcagctctgg atttgcttaa tatgcacaaa gaactatcaa aagtctcact |
| 8101 | ttcaaaacac ccgacttact acaatacgtt gaagtgtttg agcgtacgtg attttatctt |
| 8161 | tagatagatt tgaataacct cctcttttaa attgtatgta tataaggata ggattcacag |
| 8221 | atttgacata tgactaagtc atgactgaca cagctgtcat tgaaatccag gacacacagt |
| 8281 | cctggatgaa tattttatat tgacatgcat tgaaagagtt actcattgca tgtttgagta |
| 8341 | aacagagttg cttcaaatat tattgacctt aaagaaaaga aggaaggagc agaaatttca |
| 8401 | agatctcttt aaactctacc ttatttccaa aggtaaatga agtaggcagc ttctcagtga |
| 8461 | aataaaacct gatgtcgttg aaggagagta acaagtgagg ggtggaaaga gggcctgaaa |
| 8521 | ggagtggctc tattcaggaa gatgagagga tcacatgggc catgctgaag acccagctgg |
| 8581 | ggctaggaac tgcagctttc tttatagtgc ccctgacctg aacatttact gcactatacc |
| 8641 | cagcagtgct atgtgaacat agggtaggga ggacagatgg ctggattaat tcaggattgg |
| 8701 | cactttccag ggtcagtaca gctaaagaac tagagagtaa aggagttgga gttatatggc |
| 8761 | agcgtaccat gagatacaga ttggatcagg aagaaagtaa aacaaggata tttaatgctt |
| 8821 | ggaagagaag gttaaaaagt ttaaaccaac cccaaagtgt tttgttgtac taaaaaaaaa |
| 8881 | atactgcatt cataatgtta gagaaacatg aatattttcc aagtcagagc cctaatttta |
| 8941 | ctaaaataga aaggagcaaa tacaccttgt ttctttatgt acccaatcat tgggtcatgt |
| 9001 | caatccaaaa ataatcaatc aaaagttggt aagtcaatag tcatatgcga gagcccataa |
| 9061 | aatacaaagg aaaggtaatg tcatgtcctt ctcatcaagc agttgatatc caagtcaagc |
| 9121 | ttttcttcgg gtccagaaaa ggattgtgtt cattttcatg tatttgttat tcatgtagtc |
| 9181 | cttggattga gagaagaaat agtgccctag tttaaacgag ctctggctga gatgaaagct |
| 9241 | ggatagtaga atgaaagtaa acaagagaca gtgtgctgag tcaacaaata tcattaaata |
| 9301 | caactgatcc atgtgattgt gtcagtgaaa accggaaagc agatttggat aactggaaat |
| 9361 | atggacaatg tggacttggc attctgtgtg tagaaactga tattgatgta catcttctct |
| 9421 | tatccatctg tgcataatca gtatgatctt gaagatgcag tatgtcttag agaaaggaag |
| 9481 | tataaaccaa ctttaaccct gaagttcagc tagttcagct ctatcacttc ctatttttgt |
| 9541 | gatatgtaga caagttgtgc aaccattttt agtccccatt tctggtctgc aagtggaaat |
| 9601 | attaatagta tctacctttt ttgtgttaaa taaaaagata atgaataaaa agtataatgc |
| 9661 | atggcacata ccagattctt aatcactttt agatgctaat attagtaaaa acagtcaaac |
| 9721 | ttcacacaag actgttatgt cactgaatct tatgggaagg tgttaactta ataaagttct |
| 9781 | tagtaacacc ttccatattt ctccatgtct attttccaag acattctaag actcacttct |
| 9841 | ttgatcttaa ctgatctcaa cagttcatgt gacatctcca gtgtgtcacc tcattggaaa |
| 9901 | gataacatta atagtaagtc tattacctct gccacttctg catctgtgta ttgagtgaaa |
| 9961 | ataacactag tgcataaagt ttctctctta taaaagtagc acatggtatt taagattgat |
| 10021 | tttgaaaatg caaaatacat aaagtttaag acagaagaag tcatccatac ttccaggact |
| 10081 | attaacacat tggcatattt tcttccattt ttttctacat atttttaaag tccttattaa |
| 10141 | cattgtgctg cctgctattt ttacttagca ctaaagcatg agcatattct atattattag |
| 10201 | taatcttcca aacgtcatgt gtggtgacta aataatattc tgtttagata acagtgtgct |
| 10261 | agtatatgtt taatattcac ctcactgaga aaaaaaaaga cgtggatttg ttgctcttgt |
| 10321 | ttatttctgc agtaaaatac tctatagtaa aaaactctat tgtgtctgtg gccagtctca |
| 10381 | agctactatc aaataatgag cactgagttg ggaaatagca tgtgtagtag catccttcat |
| 10441 | atattatttc taccacacag acacattaca gataacctca agaacttata taataataaa |
| 10501 | atgtagttaa atcattggga atttcaagta ttaatacttt gtttttaata tatttgatta |
| 10561 | taaatgtata caacttagtt tttaataatg attgtatata acaatcacct ctcaaaattc |
| 10621 | ctaaaaaatt accaagtggc tcttgcaaac ctgcggaaga caaaacttgt gtgcctcaat |
| 10681 | cttctcccac ttgactacga ttaatagaat ttagtacaat tgatctgttt tctgcagtaa |
| 10741 | aagtcaccta tatgcctttc tatatgtgag attatttcct taggatagat tcccagagat |
| 10801 | gatgatgatg atgatgatga tattattatt attttagaga tagagtcttg ctctgtcaca |
| 10861 | caggctggag tacagaggtg caatcacagc tcactgaaac tttgaactcc taggctcagt |
| 10921 | tgatcctccc acctcagctt cttcccaagt agctgggatt acaaagtgtg tgccaccatg |
| 10981 | ccggctaatt tttaaatttt ttgtagagat gtggtttctc tgtgctgccc agcctggtct |
| 11041 | caaactccta ggctcaagtg atcctcccac ctcggcctca aaagtgctgg gattacagac |
| 11101 | atgagccacc aaccacaccc agcctgagat aaaatattaa cggtataata catttgaatg |
| 11161 | taataaagac aaggaaaaca atcaaacagc ctgggtttct ttttctaaat tatagaatca |
| 11221 | agtaacattt gcacagtgct ccatgtggac caaggacaat tttaaatggt ttccatttat |
| 11281 | ttattccttg tgacaagtta tgtggaggct atacattatc tccgtctata gctgagacac |
| 11341 | tgaggttaag tgtggttaag tgacttgcag agtcggtaag tgatggagct gggatgtgaa |
| 11401 | ttcaagtcct gtgccacatt gcctcttttt tttttttttt tttttttttg agacagagtc |
| 11461 | ttgcactgtc gcccaggctg gagtgcagtg tcgcgatctc agctcattac aagctccgcc |
| 11521 | tccctggttc acgccattct cctgcctcag cctcccgagt tgctgggact acaggcaccc |
| 11581 | gccaccacgc ccggctaatt ttttgtattt ttagtagaga tggggtttca ccctgttagc |
| 11641 | caggatggtc tcgatctcct gacctcgtga tccgcccgcc tcggcctctc aaagtgctag |
| 11701 | gattacaggc gtgagccacc gtgcctggcc gctacattgc ctcttaatgt gtagttgctc |
| 11761 | aatagtcttc cctttttttt tttttttttt tttttttggg actgagtctc actctgttgc |
| 11821 | ccaggctgga gtgcagcggt gcggtctcgg ctcactgcaa tctccacctc ctgggttcaa |
| 11881 | gtgattctcc tgcctcagcc tcccgagtag ctgggattac aggcctgcac caccacaccc |
| 11941 | agctaatttt tgtattttta gtagagatgg gatttcacca tgttggccag gaatctcttg |
| 12001 | aactcgtggt cctccccgcc tcggcctccc aaagtgctcg gattacaggc gtgagccacc |
| 12061 | gcacctggcc tagtctttct ttttaaacag taaaagcatt caacatcatc aattaacctt |
| 12121 | tgggctcatt gttaaccaca ctccacaagc ttaataaaat attgtttttg ttctttatct |
| 12181 | ctcaatatct agatgattat actattaatt gcttatttca ttcattcgtt gtttagaaaa |
| 12241 | taacttttta ttgagattga ctttctcaaa ctttatgaat tataagtttg cctccattgc |
| 12301 | attttgaaaa ttaacacttc tttggcattc tatctgctac aatttttttc tattatgaaa |
| 12361 | gcaatatgta ttcatttttt taaaaaatcc tgtaagatac agaaaaacaa aaatataaaa |
| 12421 | ttacacataa tgctcatagc aagggataag tgctcttagc attttgctat tcattctttc |
| 12481 | agtctctttc tctcatatat gtgtgtgtgt atatgtaagt gtatatatgt tgtatatata |
| 12541 | cacatacaca cacatatatt atatattata tattatatat gtgtgtgtat gtgtatgtaa |
| 12601 | ttttattttg taaattttaa gttccggggt acaagtgcag gatgtgcagt tttgttacat |
| 12661 | aggtaaacgt gtgccatggt ggtttgctgc acctgtcacc catcacctaa ataactgggc |
| 12721 | tcagcatgta ttagctattt ttcataatgc tctccctccc ccaatcccac ctgctgacag |
| 12781 | gccccagtgt gtgttgttcc cttccctgtg tccatgtgtt ctcattgttc agttcccact |
| 12841 | tataagtgaa aacgtggtgt ttgcttttct gttcctgcat tagtttgctg aagagaatgg |
| 12901 | cttccagctc tatccatgtc cctgcaaagg acataatctc attccttttt atggctgcat |
| 12961 | agtattccat ggtgtatatg tatcacattt tctttatcca gtctatcatt gatggtcact |
| 13021 | tgggttgctt ccatgtcttt gctattgtga atagtgctgc agtgaacata ggtgtgcatg |
| 13081 | tatctttaca aatttcagag gctgttattt cattgaggaa agagaagcat ttaaataaga |
| 13141 | agctctctct tcagctgcct accagcaaag gggaaaaacc tacctctctg catccatctt |
| 13201 | gtcctccttc tccctcactg tgctttaaga ggactttctc ttcctatccg tggttaaccc |
| 13261 | gtctttcttc ctctgagccc cacataaatc tgctttctca gggacctatt tccctcaatt |
| 13321 | atctcctgtc tttctccaat gtcctacatt tttgcttcta ctaggtgctt ccagtcagca |
| 13381 | tttaaaccca ctcttgtcac catactcacc tccagcacca tgcagcccct ttcttccctt |
| 13441 | ttattgttaa actctgctgc aaaaagatgt cttccctctc ctcttagttt ccacttcctt |
| 13501 | atcacaacct aacccccacc actgcaatct ggcttccgcc aaatctgcaa gcaccaaaat |
| 13561 | caacatgacc tcctcatcag gaaactcagt gggcactttt aggtccttat ttaacttaac |
| 13621 | gtctcggcag catttgattc tccctctttt gtgaaacgtg tgctactctt gggctctgcc |
| 13681 | aatagcctcc actggctttg cttacaactc tgatttcttc ctctcagtct cttttctaaa |
| 13741 | ctcttcattc tcccacccat cttttataaa tgtttgtgtt ttcttctaat tggcttaggg |
| 13801 | caccagcctc cctccgtctt caggctttca cgggtaccgt ttcctctgcc tggaacactc |
| 13861 | ttctctctcc actgcaccaa gccaaatttg aatcatcctg tgaatgtgtg tgaatgtatt |
| 13921 | gtgtagtttt tttttattcc tagttttatc aagttcagtg gttgagaaac attatttatc |
| 13981 | atgttaaaga aatactatta tattccgact ctactaatgg ttttcaagta gggaatggat |
| 14041 | agttaactct gttactgcct ttttgactgt gataggtatc ttttaaacta tgactatagt |
| 14101 | aaatcctgtt taaaggactt ttctattaaa ttatccttat atttttgcaa ggattttttt |
| 14161 | tctgcctaaa tgcatagtaa taattttgaa atttatgatt taatttaatt tgctttttaa |
| 14221 | aattttactt tagggcttta catcctgctt gacttacatt agcattgttt tattccaaag |
| 14281 | attgatttga gaagctttct atactttata ttccctatag cttttgtttg tttgtttttg |
| 14341 | agacaaggtc tttctctgtc acccaggctg gagtgcagtg gtgcaatcac agctcactgc |
| 14401 | agccttgacc tcctgggctc aagtgatcct cctgcctcag gctcccaagt agctgggact |
| 14461 | acagatgtgt gccacctgta cccggctaat ttttttattt ttgtattttt tgtagagatg |
| 14521 | cagtcttgcc atgttgccca ggctggtctc aaaacacctg gatccttcag tggatccttc |
| 14581 | tgggctcaat ggatcatcct gcctcagcct cccaaagtgt tgagattaca ggtatgagcc |
| 14641 | actgcacctg gctccttgta gcttttatgt aactggagac ttaattcttt cttaagagtt |
| 14701 | ttttaaaact tatctgaaaa actttggggc attatatttt tttcagaagt gttctttgct |
| 14761 | aatctaatct ttttcttctg tgagtattgt tgatgatttt accccaccct ttactacatg |
| 14821 | tgaacaattt tgtttttaaa attcttaatt ctttagattt tcatctttaa atattcatta |
| 14881 | ctgtttagct ctgttgaatt gtggtccaag aatgtgacat ataatctgtt tttaaaattt |
| 14941 | atttaagctt taattttcat gggcatttga aaatatgtgt catctatcag ggcttacagt |
| 15001 | tgatgtgcat atctgtttaa ttaattttac taattctgct attctgtttt actacatata |
| 15061 | taattatatt tttgtctttt gatctataaa caggattggg attttttgtt tttttgctat |
| 15121 | ctttttattg tctatttctt acagatttta ttatggttag aaaatataga ctatataaaa |
| 15181 | tcaattcttt gagctgggtt aaaatctcct atgtagcttg gtatattttc tttatgtttg |
| 15241 | aatgtttcat ctatgcatca tttacaaaat gtattctgta attgtttcat tgttttataa |
| 15301 | atgtacatta tatctagtgt taattttgat gtttatatct tctatatctt tactgtagtt |
| 15361 | ctgtttgata tattagtgtc tgaaggagat ctcttaaagg gttacactat gttaaaacat |
| 15421 | ttctccatgt actcttgtga taattttact tttgggtttg agtccatatt tttaaataat |
| 15481 | taagtttcat gattgtcata tctttttagg gcttgttcct tttatcatta agcaatgttt |
| 15541 | ctttttttct tataaatgca tatgccccta tattacattt ttcttatatt actattgcta |
| 15601 | tgtcactttc tttgggatat cgtttgcttt atacatattt tccctcccct ttatttttaa |
| 15661 | catttctgtt ttattttatt taagtgtgtc ttttgtgttt gatagcatct gtcatttcat |
| 15721 | aggtcagctt ataccattgg cacttactgt gatttctggt atatttggac ttgtttcttc |
| 15781 | catattattt tatgtcttct gttaaccatg ctttttcttg gcttatttat tcctcctctt |
| 15841 | cttcctgttt gtttttgctt ttgtttttga ttgataaagc ctttctggta cctctttttt |
| 15901 | tctctgttgt tttggaattt ttgaatttta tttatttttt agtagtaacc attaataagg |
| 15961 | ttttaacata atcatttaac catagatgct tctttctgtt tgaagttatt caatatttct |
| 16021 | gtcctcttgt ttatggcaaa gagcttagca ttttttaatt acttagcatt ttaaacttct |
| 16081 | ttcctgaact ttgatcttat agttgtttgt ctagaattta attccactct ttttaaacac |
| 16141 | aaaaatttgt gattttttaa tattccatag tgtgttaaaa ttgcaatggt atttcaccac |
| 16201 | catttatgct attgtttttt ataactcacc cctttctttc tggtataaat tattccttag |
| 16261 | tgagtttatt tactctagta actatttcag agaaagtctg tgagtggtaa acactctcaa |
| 16321 | tatttgttta taagtgttct gagtttaaaa ttattaccta agctagctgt gtaattctaa |
| 16381 | gctaacagtt atttttacgc agtcatgtaa aggtattagc ctattgcctt ttggtatcaa |
| 16441 | ttgttgttgc tgaataatct caggttaccc aaaattggca ttcctattaa ggaattaaac |
| 16501 | tttttctttc tggtagagtt taagatttta tttatcttta atttttacag ttttaattca |
| 16561 | atgtgtccaa gtatgaattt ctttcttttt ttcttttttt ttttttcttc tgagacagag |
| 16621 | tctccctcta tcactcaggc tggagtgcag tggtgcgatc tcagctcact gcagcctcca |
| 16681 | cctcctgggt tcaagcgatt atagtacttc agcctcccac atagctggga ctacaggtgt |
| 16741 | gcaccaccat gtccagctaa attttttgta tttttagtag agacagggtt tcaccatgtt |
| 16801 | agccaggctg gtcttgaact cctgagctca agtgatctgc cctcctcggc ctcccaaagt |
| 16861 | gctgggatga caggtgtgag ccaccatgcc cagctgtatt tcttttttaa atctgtcttg |
| 16921 | tcatttggtg ggtactttcg accttaatat tccagggttt gtttttcagt tttgaaaaat |
| 16981 | tatcagccat ttatctcttt atatattgct tctaggctgc tcccctcttt tatttttctc |
| 17041 | aaactcctat cagaggtgta ttagtgcctg tcactgtata ctttatatct cctaactatt |
| 17101 | ctttaatagt gtccatgtct gtatttcttt gtattatgtt ctgtatatta taatgatcct |
| 17161 | tatgatatat atatcatata tatgatatat ataatgatcc ttataatata ttatattata |
| 17221 | tataatatat attatatatt atatattata taatatatat tatatattat atatataata |
| 17281 | ttatatatat tatataatat atattatata atatatatta tatataatat atattatata |
| 17341 | tattataagg atcgttataa tatatattat aaaaattatt ataatatata ttataacaat |
| 17401 | ccttataata tatattataa tgattattct gttgaaaagc tgggaagaaa gtggccctga |
| 17461 | tgactgtgta taagatgtca ttttacataa aaattctgct ttcttatttc aactactatg |
| 17521 | tttcaacttt tttatttact ccttttgtga aggccttata aatacagttt tgttcattta |
| 17581 | aacagtacat cagtcactgt gctcaccctg ggaattaaat gatgtctaaa acatgatccc |
| 17641 | tgacctaaag ctcaaggttt taataattga gtgtgggtca aagacgtaaa cagatcattt |
| 17701 | taatagtatc tgtgtgacaa gtggcattat aagaatgaca tttcaggata acataaggtt |
| 17761 | aaggaaagag gcatttgggt ctgcctagca tgtcagggga agtggaagga gatggatgct |
| 17821 | taaactgaat cgtaaaagac aaaataacat tagcaaggga gatatggggc aagtaaaacg |
| 17881 | ctcccactca ccattttttt tcactctcag cagatgagct tgcctttgac tttaccaaaa |
| 17941 | aataataata ataataaggt ttattcagaa agtggatgta tcttttatgg ccaaacaacg |
| 18001 | tgccagtctg ttctagatgc tctataagtc tgtcactgac tatttcactt ctaaatgtca |
| 18061 | atcatactac ttatttggct ttgatgtata ccttgccctt taactgaagg gaccatataa |
| 18121 | tttactgtat aaatgcaggt agccctcatt tcacagagga gtgagaaaaa agaaaggacg |
| 18181 | atgtctatag cctttcctcc tgttgtctta aaaaaaataa aatagaacaa aacagctttg |
| 18241 | agatctaatt cacataccgt gcgctttacc cattaaagtg tacaattcac aaagtggtgt |
| 18301 | ctcttcattt tctagttgta tgcccaagtg aattctctat gaatctgatg aagcttaagc |
| 18361 | ttcagattct ctcatttgca ggagcccttt ctaagaccct cagtgggctt tagcaatgtg |
| 18421 | cttttatgag tgtctatttt tataaaatta ggggaaaatt gttcttaaaa aacaattttt |
| 18481 | taaattatta tccttttcca ccctgacttc ccttcagact cacttccctt tatgctgatg |
| 18541 | atattggggt ggacacagaa attttgtatt cataatttta tattttttct ttaaaaagta |
| 18601 | caaattgcac aaacttgggc cccataaaac ttggatctgc ctctcctcgt atatcataaa |
| 18661 | ctctaagccc tgctctcctg agagacttct cttttgattt tcaaatgcct agtttggaag |
| 18721 | tagaaacaac attctttaca taagctcttc ctttcttttg gcaaatttct actctgggga |
| 18781 | aacagaattc tcattcaggt atgtaatggc ttctgactgc ttaaggaagg gttagtaaac |
| 18841 | aaaattctag aatgttctct tttgatttct cctttccttt cagctcctgt catcaccact |
| 18901 | cctcttgaaa cagtggatgc cttagttgaa gaagtggcta ctttcatgtg tgcagtggaa |
| 18961 | tcctaccccc agcctgagat ttcctggact agaaataaaa ttctcattaa gtaagtattc |
| 19021 | cacattttaa tttttttaaa tttttgtggg tatatagtag gtgtatatat atgtatatat |
| 19081 | ttatgaagta gatgagatgt tttgatacag gcatgcaatg taaaataagt acctcatgga |
| 19141 | gaatggggta tccatcccct caagcattaa tcatttgaat tacaaacaat ccaattacac |
| 19201 | actttaagtt attttaaaat gtacaactaa gttattgttg actatagtca ctctgttgtc |
| 19261 | ttatcaaata gtaggacttt ttcattcttt ctattttttt tgcacccact aaacatcccc |
| 19321 | tactcccccc gatccctgta ctaccctttc cagcctctgg taaccatcct tttacgctcc |
| 19381 | atgtccatga gttcaattgt tttgatgttt agatcccaca aataagtgag aacatgcaat |
| 19441 | gtttgtcttt ctgtgcctgg ctaatttcat ttaacataat gatctccagt tccatccatg |
| 19501 | tctttgcaaa tgactggatc tcattctttt tatggctgaa tagtactcca ttgtgtatat |
| 19561 | gtaccacatt ttctttatcc attcatctgt taatgaacac ataggttgct ttcaaatctt |
| 19621 | agttactgaa aacagtgctg caacaaacat aggagtatag atatctcttt gatatactga |
| 19681 | tttcctttca tttgcttata tatgcagcag tggggttgct ggatcatatg gtagctcaat |
| 19741 | ttttagtttt ttgaggaaac ttcaaactat tctccatagt ggttgtacca atttacattc |
| 19801 | ccacatcaac agtgtacaag gattccctta tctacacatc cttgcgagca tttgttattg |
| 19861 | cctgtctttt ggctctaagc cattttcact gaggtgagat gatatcttat tgtagttttt |
| 19921 | atttgcattt ctctgatgat cagtgatgtt gagcactttt tcatatgcct gtttgccatc |
| 19981 | tgcatgtctt ctttcgagaa attgttcaaa cttttttgcc tatttttgat tggattaata |
| 20041 | tattttttac tatagagttg tttgagctcc atatatattc tggttattaa tcctttgtca |
| 20101 | gatgggtagt ttgcaaatat ttcctcccat tctatttgtt gtctcttcac tttgtttatt |
| 20161 | gtatcctttg ctgtgcagaa ggccttttaa cttgatgtga tcccatttgt ccatttttgt |
| 20221 | tttggttgcc tatgcctgtg ggtatctctc aagaaatttt tccccagacc aatgtcctgg |
| 20281 | agagtttccc caatgttttc ttggagtagt ttcatagatt gaagtcttag atttaaatct |
| 20341 | ttaatccatt ttgatttgat ttttgtatat ggagagagat aggggtctag ttttattctt |
| 20401 | ctgtacatgg atatccagtt ttccacacat catttactga agagactgtc tttttcccag |
| 20461 | tgtatgttct tggtacctct gtcaaaaatg aaatgagttc actgcaggtg catgaattta |
| 20521 | tttctgggtt ctctgttttt tcccattggt ctatgtgtct cttttgatga aagtactgtg |
| 20581 | ctgttttggt tactctagct ctgtagtatg atttgaagta aggcaatgtg attcctccag |
| 20641 | tttttttctt ttttcttttg gctattctgg gcctttcgtg gtttcacata aattttagga |
| 20701 | tttttttttt ctatttctgt gaagaatgtc atcggtattt tgataggaat tgcatggaat |
| 20761 | ctatagactg cttttggtag tatgaacatt ttaataatat tgattcttcc aatccattaa |
| 20821 | tatggaatat ttttccattt ttttggtgtc ctcatgaatt tctttcattg atgtcttaca |
| 20881 | gttttcatta tagagatctt tcacttcttt gattaattcc tagctattta attttatgtg |
| 20941 | tggctattgt aaataggatt atttttaaat ttatttttca tattgttcat tgttggtata |
| 21001 | tacaaatgtt actgattttt gtacattgat tttgtatcct gcaacttcat tgaatttatc |
| 21061 | agttctaata gttttctgtg gagtatttaa gtttttccaa atataagagc atatcatttg |
| 21121 | taaacaaaga taattcttcc cttccaattt ggatggccct taatatcttt ctcttgtctg |
| 21181 | attgctctag gtaggacttc caggactgtg ttgaattaac agtggtgaca gtgagcatcc |
| 21241 | ttgttgcatt ccagactaga ggaaaggctt tcagtttttc cccattcagt atgatactac |
| 21301 | tagctgtggg tctgtcatat atggctttta ttattttgag gcattttcca tctatttttt |
| 21361 | gagggttttt atcacaaagg gatgctgaat ttttatcaaa tgctttttca gcatatattg |
| 21421 | aaataatcgt atgtttctta cccttcatct gttgatatga tgtatcacat tgattgattt |
| 21481 | gcatatgttg aactatcctt gtatcccagg gataagtttc acttggtgat gatatatgat |
| 21541 | ctttctaatg tattgttgaa tttggtttgc tagtattttg tgcagtctgg ataggccaat |
| 21601 | aatcccaaat catcaagtcc tggttccttt tttgcttagc agtttttccc acttatcttt |
| 21661 | tcctcttgaa ttttactata accagaaaaa agaaatgagg ccgcagcttc gacacatttc |
| 21721 | ttagaaattt cctcagctaa atatctgagt tcatcattca caagctttgt tttccacata |
| 21781 | actgcaggac accattcagc taagttctct gtcctgcata acaaggatcc ccctttcttc |
| 21841 | ttttcccagt agcatgctcc tcatttccac tcaagtcctc accagtagtg ctttgaacat |
| 21901 | acatatttct acgaatagtc tgttcatgat aatttaggta ttctttaaga tgatgtagat |
| 21961 | tttctctacc atggaactct cttccttcca attcctcagg agcagaatca ttaacacacg |
| 22021 | tatttctatt aacagtctct ttgaggaaat acaggctttt tctagcatgc ttttcaaatt |
| 22081 | tttttcggtt ctaactattc atcaattgca aagccacatc cacatgttta ggtatttgtt |
| 22141 | gcagcagcac cacacttcca ggtaccaaaa cctgtattta tttattatcc ttgctgtaac |
| 22201 | aaattaccac aaacttagtg gcctaaaaca acataaacgt actatgttac agttctggag |
| 22261 | ttcacacgta tgaaataaat ctctctgggc taaaatcaaa gtgttggcag ggctgagttc |
| 22321 | cttctggagg ctctagggaa gaatctgatt tcttgccttt cctagcttct agaggccacc |
| 22381 | tgcgttcctt ggccccctac tccacttata agtctagcaa tggcactact ctgacctctg |
| 22441 | cctccattgt catacctccc tttatcaatc tgcctctctc ttccatcttt taaggatact |
| 22501 | tattatcaca cagggaccat tcgaatagtc cagaacactc cattttaaga ttcttaacat |
| 22561 | ctgcaaaata aggtaacata ttgacagatt ttggtgatta aaacaagaac ctctgtggga |
| 22621 | gtgttattct gcttaccaca cgcatttggc ataaaaaaac tcttatttgg ttaaaagaaa |
| 22681 | ctctctttta tattgctgct tctcttccta tttctttcta ctgccctaat tcccttctca |
| 22741 | acaaatggtt tgcatataat taaataggta atatatgtaa agcatatatt tttatagttg |
| 22801 | gaaattgttt tcctccctta tgtatcccaa gtaactagaa gtgtgcctgg cacttgttag |
| 22861 | caatcagtaa gtaattgttt aatgaaagaa taaatattca actaatcatt agttacttct |
| 22921 | ataagtaatt aaccctttct gttcatcgta tttatacata tgcatgttct ttatctatta |
| 22981 | atagctgcta cttcccagaa cttattggga gaagacacct atgtcttggt tttatatgct |
| 23041 | tttctgaata aagtaaactt tacattttgt cttatcagtg ggcatcaccc tttgaaagca |
| 23101 | tttttgaagg actatggtac tttgactatt ggagcctaag aaagatgaga atataatgtt |
| 23161 | agctttatat tatcaagtaa ggttattcta ttattcagaa gtcactcaag tttcctctca |
| 23221 | cctttattct acttcctcat taacaagtca tcggtttgat acattaatca tggcaaaaaa |
| 23281 | atcacagagg aagcactaat ctgtcatttt tttctgtgac ctgcagactc tttgacaccc |
| 23341 | ggtacagcat ccgggagaat gggcagctcc tcaccatcct gagtgtggaa gacagtgatg |
| 23401 | atggcattta ctgctgcacg gccaacaatg gtgtgggagg agctgtggag agttgtggag |
| 23461 | ccctgcaagt gaagatgagt gagtgggaaa cagattcgtc tattgatttg aaagttgact |
| 23521 | tggtacactt agttttgtat ttatttttta catttttttt ttgagacaga gtctcactct |
| 23581 | gttgctcagg ctggagtgca gtggcatgat ctcggttcac tgcaacctcc acctcctggg |
| 23641 | ttcaagtgat tctcctgcct caggctcctg agtagctggg actacaggtg cgtgccacca |
| 23701 | agctcagcta atttttctat tttcagaata tacagggttt cactatgttg gacaggctgg |
| 23761 | tctcgaactc ctgacctctg gtgatccgcc caccttggca tcctgaagtg ctggaattac |
| 23821 | aggcgtgagc cactgtgccc ccgctgactt ggtacacttt aaaacagtca gtaaatagag |
| 23881 | attttctcag gcctggtgat tcagcactga gacaagttca agttcaagca caaccctgag |
| 23941 | gccaattaat cccacacctc ttatgcctgc tatcaaccac taccctaagg caagcctctc |
| 24001 | aattctctat cctctcccat tttcttacct tcactcatct cctgacttct tccaccctga |
| 24061 | attctttcac caggaaaact tcctctgaaa catttaccct acacctgtga aacacaacca |
| 24121 | aacacacttt ttatttctaa cctctttgca gacacttttg ccttatttca aatttgatat |
| 24181 | tcttctgagt gattttctgc ttcagtggaa gctgttctat tttctccact tcatgtctac |
| 24241 | aagactgcca aacactagta ggtaatctcc cagccctcag tgttgctttt aaaccattct |
| 24301 | attttgtatt cctatacaaa atctttcctc tgatgagggc ttatgccatt ctgacctatc |
| 24361 | ttttctctaa ctgtattcat catgtcatct gctcttctag gcacttcccc atatttatta |
| 24421 | aaaactttga ttccaggctt actatctttc tatccatctc tactcctgca attaccctaa |
| 24481 | gaaattttaa cagccatgaa atagaaccac tagccactta attctcataa ttccttgacc |
| 24541 | tgcagaaatc cagtgatgtt tgtctttttt tttcaatttt aaggctcata cttttaatct |
| 24601 | ctatgtacac acgtatgtag tcgttaaggt aagtctgcca ctgtgaggaa aggtagtttc |
| 24661 | attttatttt tttaactttc attttgagtt caggggtaca tctgcagaat gtgcagttgt |
| 24721 | tttacatagg taaacatgtg ccatggtggt atactgcaca gatcatccca tcacctaggt |
| 24781 | attaagccta gcatccattc tattcttctt gatgttctcc ctccccacac caacaccccc |
| 24841 | gacaggcccc gtgtgtgctg tttcccccca tgtgttcatg tgttcccatc gttagctccc |
| 24901 | acttctaagt gagaacatgc agtgtttggt tatctgttcc tgcattagtt tactgaggat |
| 24961 | aatggcttcc aactccatcc atgtccctgc aaaggacatg atttcattcc attttatggc |
| 25021 | tctgtagtat tccatggtgt ttaggtacca cattttcttt atccagtcca tcattaatgg |
| 25081 | gcatttaggt tgtttgtgtt aactgtttta ttgttttttt gttatatata tttatatata |
| 25141 | aatatatgta aacatatatt taaatataaa tacatacata tatttaaata tatacaaata |
| 25201 | tatacttaaa tatataaaaa catatttaaa tataaatacg tataactata tatttaaata |
| 25261 | tataaatata tagctatagt tatatatagt tatatattta catttaaata tatagtttta |
| 25321 | taatttattt atataaacta cataaataaa ctatatattt atataaaata taaataaact |
| 25381 | atatatttat ataaatatat catatatata ttcatatttt atataatata taatatataa |
| 25441 | atatataaat atataaacta tatatttata tattgtatag ttatatttat atataaatat |
| 25501 | atagctatat atttatattt aaatatatat taaatatata tttaaatata aatatataaa |
| 25561 | tatatataaa tacatattta tatatatgta aaaaatataa aaatatgtga aaaatacata |
| 25621 | tttatatata atatatgtaa aaaatattaa aatatgtaaa aaatacatat ttatatatat |
| 25681 | gtaaaaaata taaaaatatg taaaaaatac atatttatat ataaatatat acaactatat |
| 25741 | ataaatatat agttatatat atttatatat tatatatatt tatatatttt ttactacata |
| 25801 | atatataaca tattatatat tttagtatat atattatata ttagtatata ttagtaatat |
| 25861 | accatataat atacatagta tattatatat aactatatat agttatatat aaatatataa |
| 25921 | ctatatatgt tatatatagt ttatatataa tattatatat tatatataaa ctatatatat |
| 25981 | cacatatagt ttatatattt tttaatatat aatatataac tatatattta aatatataaa |
| 26041 | tatgtaacta tatatttata tataactata tatttaaata taaatatata tttaaatata |
| 26101 | aattatataa atatacataa aaatacatat ttaaatataa tatataaata tatatttaaa |
| 26161 | tataaatata tatttatata ttatattata aatattataa ttatatatta tatattataa |
| 26221 | atatataata tatattatat attaatataa gtataaatat ataaatatat attatgtaag |
| 26281 | tataaatata taaatatata ttatataagt ataaatatag aaatatatat tatataagta |
| 26341 | taaatataga aatatatatt taagtataaa tatataaata tataaatata tatttaagta |
| 26401 | tatataaata tatataaata tatatttaag tataaatata tatttaagta tatataaata |
| 26461 | tatataaata tatatttaag tacaaatata taaatatata taaatatata tttaagtata |
| 26521 | aatatagaaa tatatattta aatataagta tatatatatt taaatataag tatatacata |
| 26581 | aatatatata tttaaatata agtatataca taaatatata tatttaaata taaatatata |
| 26641 | tttcaatata agtatatata aatatatatt taaatataag tatatatata aatatatatt |
| 26701 | taaatataag tatatatata aatatatatt taaatataag tatatatata aatatatatt |
| 26761 | taaatataag tatatatata aatatatata tttaaatata agtatatata taaatatata |
| 26821 | tatttaaata taagtatata tataaatata tatatttaaa tataagtata tatataaata |
| 26881 | tatatattta aatataagta tatatataaa tatatatatt taaatataag tatatatata |
| 26941 | aatatatata tttaaatata agtatatata taaatatata tatttaaata taagtatata |
| 27001 | tataaatata tatatttaaa tataagtata tatataaata tatataaata taagtatata |
| 27061 | tataaatata tatatttaaa taaatatata tatatttttt gagacggagt ctcgctcagg |
| 27121 | ctggagtgca gtggcatgat catggctcac tgcaacctct gcctcctgag ttcaagcaat |
| 27181 | tctcctgcct cagcctccca agtagctgag attacaggca tgtgccaccg gaccctgcta |
| 27241 | atttttgtat ttttggtaga gtcagagttt cgccaagttg gccaggccag tctcaaactc |
| 27301 | ctaaccttga gtaatctgcc cactttggcc tcccaaagtt ctgggattac aggcatgagc |
| 27361 | ctgtttgtgt taactcttta tcagcgtagc caatcgatga ctcactcttg gccttgtcac |
| 27421 | ccactggaag tagtctgcat ttaaaatctt cagcttcagt atgtcactct catcatggac |
| 27481 | attttttggt acagccttgt cagatgagca aaacagtttc ccatactcat gatttgcctc |
| 27541 | caagtagagt ttgtgggcac aaacttccat tgtctccttc tcagctaaca acagtgtctc |
| 27601 | agtctctctc attctcctac tcttgctata caacctcaac atcaccagca tcatcatgtt |
| 27661 | ctcttacctt gattcgtctc tgttctcctt tccattgatt ccccaatggg gcttttattt |
| 27721 | ccttccatac ttagttcttt cttctacctg gtgatgtatc atcaaacact gccttaccag |
| 27781 | catctttaaa tctcatggcc tcctgtcctg taagcatctg ctacttgcaa actgtcaatc |
| 27841 | ttacaatgat gtaaccttca tcttctcttc tctgacatgc ttataaccat acatacgggc |
| 27901 | accacacaaa ttagaggaat ttaggtctac ttgggacatt tatttttagt ttttgtgttg |
| 27961 | ttgtctttcc tcattatttc ttatcttttg ttattttgct ggaggaaatc attaggtact |
| 28021 | tttttcagag tgtgaagatg taaaatattt cctgaatttg tgcacgattg gaagtcttca |
| 28081 | ttttgctcgc attgttggat aataattttg ctgcatgcag aatgagagtt ttaaaatccc |
| 28141 | ttttctttag ttttagtaga tatcctctgt tgcagatgag aagtcttttt cttatttctt |
| 28201 | tacaagtaaa tctgatgtct ctcttaaaaa gtttaggatt tttctttttt ttctagagtt |
| 28261 | cataaatttc acaaattgac aagaatatat taggtgtatt tttaagaatt attgctactc |
| 28321 | agttttaggt gggccttgtt tttttgtttg tttggttggt tggtttttgt tttgttttgg |
| 28381 | tttttttgag acaagatctc actctgtaac tcaggctgta gtgcagttgt gtgaccatgg |
| 28441 | ctcactgcag ccttgactgc ttgggctaaa gcaatcctcc tgcttcagcc tcccaagtag |
| 28501 | ctaggactac aggcatgagc caccatacca ggctactttt atttatttat ttactttttt |
| 28561 | ttggtagaga taagttctca gtgtgttgca catactgagt cttgaagtcc tgtgctcaag |
| 28621 | tgatcctccc accttggcct tccaaagtat cgagattata ggagtgagcc actgcaccca |
| 28681 | gcctaggtgg gcctttttga tctaaagacc aaagcttttt agaaaggtac attttctcta |
| 28741 | ttttttttct tttttctttc tctatgactt caaatttttc ttctcatatt ttctgcatgt |
| 28801 | cttaatattc tactctcttg aagattcctt ttactttttc ttcatattat gaatgtggtc |
| 28861 | tccagtatga ctattctgtc attctgagtg ttttggtctt aaaaactata tttttaattt |
| 28921 | ctaagaattt ccttgatgtc tctgttgtca taacaaactc acttgtatga tacaatattc |
| 28981 | acttaagctt ctaaggatat cactagtgca tcttttaaaa agacattttc ttttttcccc |
| 29041 | tgcattactt ttatttcctt cagaaaaaaa aaaattgatt ttgtttgggt tccatccttt |
| 29101 | tatgtttaca atttttctca aattgatctt ttattttttg atcataataa atatacatgg |
| 29161 | agtgttcagt taattaggat aaaaagctgg tgggaatagc atgactttat ggaatactga |
| 29221 | tttggtactt actgtgagta agtaggtggg gatctgccta tatgcaggct tcccttcagt |
| 29281 | atgcagagaa gtaagaaatc aaataaggaa gttggaatgc tctcatttgc taaataatga |
| 29341 | agacttcatt ctgaggatga aacacattca agtatttcct tactttgtga cactaccttt |
| 29401 | cttttgaata tttttgcata ttctctctgg cccaattgtt cttgatttgg gttctatact |
| 29461 | aaatcccctt ttcttttctt gccttctttt ttccaacctc tcctttggag atctcatcca |
| 29521 | ttcatgtgga tatacaccgg tgtggccatt ccaattgcta aaattttgga atgcttccat |
| 29581 | acttcttgga aagatccgtt ttataaggct cccgagatcc cccaacccct atcactactc |
| 29641 | cagctctctt aagccctccc ccaagagctc cctcccctaa attcctaatg gtccatcagc |
| 29701 | taaagtctca atgctggcac agcgggatac ttccagcatc ctgctttaag gtgaccagtt |
| 29761 | ctgattggtc agtacttatg actccaccct gaatcatgtt cccttacagt ggggatccat |
| 29821 | ggctatgcgt caatacccta gtcattccta gttgacttta cattcaattt cccaaaaggg |
| 29881 | aattcctcac ccctcaccta acagatgacc aagacatcaa gtcactgaga agataggaga |
| 29941 | tttttgcttt aagcaaaggt ctttacctaa gacattatct gtgcctcagc tttaccatag |
| 30001 | agatcttgtt cagggaacct atggaaggca tagataacac tttatttgac tgtcatagga |
| 30061 | atatcaagtg ctcagctaag tttttcccag caactgtatg acaatacaac tcacaattga |
| 30121 | ggaacctggc cagtactgta tttacatgta ggtatttaca gtttattaga aggagagttg |
| 30181 | tatgctatag atagcaggca aagtaataaa gttgttattt tcctatattt taaagccttt |
| 30241 | cttgtatttt tgctataaca cagggccgca ggaactctaa atgacagtga atgcttagat |
| 30301 | taaaaaccaa caggtctcac gcctgtaatc ccggcacttt gggaggccga ggcgggcaga |
| 30361 | acacgaggtc aggagatcaa gatcatcctg acaacagggt gaaaccccgt ctccactaaa |
| 30421 | aatacaaaaa aaaaaaaaaa aaaaaattag ccgggtgtgg tggtgggcgc ctgtagtccc |
| 30481 | agctactcgg gaagctgagg caggacaatg gcatgaaccc gggaggcgga acttgcagtg |
| 30541 | ggccgagatc gcgccactgc actccagcct gggcgacaga gcgagactcc gtctcaaaaa |
| 30601 | aaaaaaaaaa aaaaaacaaa aaaacaaaac ccaacaggtc atgaaggctt tgcaagtatc |
| 30661 | tcttaccacc aacaacacca aagcccccat tcctctccct cacattgaag tagaagaaaa |
| 30721 | caaagtccat atttcatgcg ttcctttgtc aaaataacaa attggttacg gcacatgtcc |
| 30781 | tgcaagaaat aaaatgtgtc cattgctcag tagtctctct tgcgaagtcc ccctctgtat |
| 30841 | ctgtaacgta aaattcctaa ggacataaat gtagttttaa ctattttacg aatagtacta |
| 30901 | aaataactgg catcctgaac ttatcaattc ttttgtatat tagatttaat aactttcttt |
| 30961 | gaaagtttca tatttgagaa taaaaattaa ctggcataaa ctatttcttc tgaaagatta |
| 31021 | aaagtgtttg aacagtaaat tacatgtttg agtcccccca aattgtatga cagaatttct |
| 31081 | tctgattgtt aatataataa tatctggttt actttctaga gaatgaatgt atttaattaa |
| 31141 | taatcataaa atttaatagt caaattaaga tggaagctaa agggaaaata ggtcataatt |
| 31201 | tctaactagg taagaatata gttatatcaa aattttcttt tcaaatgttt tatgtctatc |
| 31261 | agtatgaatt aaataatgtt gaattgctat ggtgacagtc tatgttaatc agcttttatt |
| 31321 | aactatcttt taaaatgaag aatgctatgg taacttcaca ctgttaatat ttttcataaa |
| 31381 | tttgtgtttt gctagtaaga ttctaataaa gcagagacaa ccaagtgttt ctaaactgat |
| 31441 | ttctgtttga gatctactga ggaaaactgt ctttgcattt ggtgaatttt gtaatgtaac |
| 31501 | atgcctcaaa tgattctcaa aacagatgtt tagaattaaa ttgaaagtta gaaactctag |
| 31561 | gtttaataaa gccatattgc cttatttatt ttgaattttc atttcttttt agaacctaaa |
| 31621 | ataactcgtc ctcccataaa tgtgaaaata atagagggat taaaagcagt cctaccatgt |
| 31681 | actacaatgg gtaatcccaa accatcagtg tcttggataa agggagacag ccctctcagg |
| 31741 | gtaagtggtt atgatgttaa aacacatata taaaatgtat tttaaaatat aacatttctt |
| 31801 | tacacactca gttacacact tacaaacttc tagtataaag tagtttccat gtacatttta |
| 31861 | accaaatgta aatatttctg tttgcatata tgcaaactgc atggcatttc atttgggtct |
| 31921 | ggctagggaa aaaagagatg ggaaccaata atgttaaaga tagaaaagga ggcaatgaaa |
| 31981 | aagcaaacct ggaatccttt gtatttgttg gctggctaaa tcatttattt ttttttaatg |
| 32041 | ttgaatctga gacttagggg atgaaaaaga aaaactaaaa cccaggcttt agaactgaca |
| 32101 | gtttctggaa ggagggaggg agacaaggtt cggaagaaaa gaaatgagat ctctggagag |
| 32161 | aagttaggcg aaaagactca agactcatac acaaatacct tggcaaatcc ctcctgagtc |
| 32221 | atgaagagta gtcacgcctg taattccagc acttcaggat gccaaggcct gaggtcagaa |
| 32281 | gttcaagacc agcctggcca acatggcgaa atcctgtctc tactaaaaat acaaaaatta |
| 32341 | gctgggtgtg gtggtgtgtg cctataatcc cagctactca ggaggctgag gcaggagaat |
| 32401 | aacttgaacc cgagaggcag aggttgcaag tgagccaaga tctcaccact gcactccagc |
| 32461 | ctgggcaaca gaatgagact ccatctcaaa aaaaaaaaaa gttattatcc atatgcttat |
| 32521 | tttgtactgc ctctgtatct tatccccaca tttattgata aattattatg aggcatcatt |
| 32581 | attgtctttc catctttata tcacctaaaa gattacagac tatgtttttc atttaacaat |
| 32641 | gcatagtata ttttaagagg aatacaaagg tgtcacttta tgtcaattaa ctgaactaag |
| 32701 | gtataaaatt gctcagcttt gctttattta aaataaaata aatattgcta atttttaatg |
| 32761 | attaattatc tggctttcct cttgttataa atcttggtgc atttcctaca atgacaaaca |
| 32821 | agggtggttg tacttatgtt actgaattta gggttcaaaa cagaaaagac agatgtaata |
| 32881 | tttaaactcc aaagagtaat tttaatatta tgtgtgaaat tttctagtat ttcaaaggac |
| 32941 | atgtactctt ctaaagatac catctagggt gcaaggtaaa cctgatagtt aaaagcaatg |
| 33001 | tgtttattta ttcatatttc aatagctttt ggggtactag tggtttttgg ttgcaaggat |
| 33061 | gaactgtata gtggccaagt ctgagatttt agcgtgccta ttacctgagt gctgtacatt |
| 33121 | atacccagta tatagttttg tatgtctcac caccccactc tgcaacattt cccccttcta |
| 33181 | aaagcaatgc ttttaatcag atttagctgt gactttgggg aagcaaaata agcccatata |
| 33241 | catatatata ttatacatat acatattata tatataatat atatatacac acactcatgg |
| 33301 | gatgaaagaa aataaaaata aactatacct cataggaccc tggtgaagat taatttaggt |
| 33361 | actcttgcac tatttcattt aatattgtat gttttacata ataagtggcc aataaaggtc |
| 33421 | aaagcgatat ctttgatgat gataatagtg atgacaataa gttgatgata tttggcaaat |
| 33481 | gtatccttga tagacccata aacatttttg aattcacgtc cctatctctg gcaggaaaat |
| 33541 | tcccgaattg cagttcttga atctgggagc ttgaggattc ataacgtaca aaaggaagat |
| 33601 | gcaggacagt atcgatgtgt ggcaaaaaac agcctcggga cagcatattc caaagtggtg |
| 33661 | aagctggaag ttgagggtaa ggagctgcat ttcttcccct gactgtgtga ccaggggcct |
| 33721 | cactgtctac tgtgaaggct gcacctgggc ttgggagaga agagatgtgg gcagcccact |
| 33781 | tccctcagtt gttccagata ttaggccata agttcaagcg tatgataaca gaaaaggaaa |
| 33841 | actgaaaaaa gaaagaaatt ttcactagta taaaaggtca tattccattg attactcaca |
| 33901 | atcactggta atttttacca aaacagacta aatttactgt aacagtaaaa taatagaaac |
| 33961 | ccattttaga atgccttcct ggtttggaca attcttccct catttaaaga cttgggcata |
| 34021 | ataattttaa tctttttaat catacagaac aactttaaaa agatgtgata aggaggaaaa |
| 34081 | agtagctatt ttcagatagt gttctatcac aaagcttgaa aaaaaaaaca ttaactcaaa |
| 34141 | tcgtaaagcc aaaaataatc ccagttctgt gagttatgtt atcaaaatcg tttttctttt |
| 34201 | ctttctgaaa atatttttta atcagctaat tcaataactt ggtgtgaaag gtcttcactg |
| 34261 | agaaatcaga atccaaatag gtctaggtgt tctatttgct tgttagtcgg gtccttcttg |
| 34321 | acctaaaatt gcaccacatt ttgcttaaaa agaactatca gcacacctct attaggcgaa |
| 34381 | taaactgagt acaaaccctt cctagctctt cctgttgtat gccagagaat taacattttt |
| 34441 | ggctcctagc tttctcacct gttaagtggg gtttgctcta tttaatgaag ttaggtgttg |
| 34501 | atagtgttta gcagagcatc tggtcctttg tgcaccctca ataaatgttg gctataagaa |
| 34561 | ttatcatcta tttttctgct tttctcttat ctgattgata agtgttagag tatcacatac |
| 34621 | tggcacaaat tacagttcaa tcaggaaaag acttgaagag ttctccatat tttactttga |
| 34681 | ttctgtccta ctttccttag gccaaaatct atctttttct gcatatttcc ttctcattca |
| 34741 | tatagttttc tattaactcc tttaagatta tttttatttc cttgatggac attcctgtca |
| 34801 | cagctttaat gatctcgttt cccacgtttg cctccaattg aggtaacatc tttattatgt |
| 34861 | cattgtacca attgctcata aaattttcat taattcctca tggaaaataa tgaggttggt |
| 34921 | gggaatagtg gctgtcagaa tataaaaata cattttcatc aagttgctgt aatttaatat |
| 34981 | cttctaaata atcagtgcta tttgaagctc cgtattcaac aaccagttga tatgatttat |
| 35041 | tcagtaaaaa ctaaataaac ctacaaaccc acccagacac acattcagac tcactgaata |
| 35101 | tctgtctgcc tagatccagt gttttcttgt tctgtctaag gaatgatgca gaattaatgc |
| 35161 | tatccatttg tataaaatat cacaaagagt ttttctcttt aagatattaa aaataagctt |
| 35221 | tctgtattat aatatttaat actgatcatt taggtcacta gcttcataga tattaaaaca |
| 35281 | acttgcttta cctttgacct agagaaaatt atcaccgaac tccattagtt ggagttatta |
| 35341 | tacagtttgg ttttgtgtta ccatttaatt ttgtatcatg cctgtcagta acattgaata |
| 35401 | ctgcctcttt tctcgtaacc tttaaatggt catgcttagt gctttaaaag ttaatatatg |
| 35461 | ctagagatag tattaaattt gctagaactg agagctcata atatcattta aagttattct |
| 35521 | aactgtaggt aagatgattt tcgcacacat ttagcacaca tgcctatttc taccctcatc |
| 35581 | ttagtcccta ttaaaatagc caaaatatgt gacatgctta gcacaattgc tggtacataa |
| 35641 | taagcattta ataaatggaa tctactatac ttattatgat taaaacataa aattgtaaca |
| 35701 | aatttgcttt ggctctggaa gctctgaaag gttgacttcc acttgaataa atagttgagg |
| 35761 | tattttcata agatataatg tagacgtgac ctatttagag gtaaggctaa ctcaacctac |
| 35821 | aactcagaac aagttttttt ttaaaaaaaa gcaactgaga aatgactgga aaaacctcaa |
| 35881 | aagtacatgc agatgcctgc tagtttggca atgatgagga ctgaagatga tgaggcaggg |
| 35941 | ccatcgtcag ttggggcaga cgagcatatg tatgtatcaa gtcgtaactc tgcaaattag |
| 36001 | ttatgcagtg actatatcga atcagaactc tatggtgcca tttgatgctg ggctacatgt |
| 36061 | aacacagatg ggcatacagg aaaagataga gttttgtcat tgatagcctc taataaagat |
| 36121 | aaacatgcca ggccatggat tgggtaccat gtgttaacta aatctttgtt gttacaactg |
| 36181 | tgaaagaaac accctaccac acagaagtaa agctcccttt ccagattgcc attctcagac |
| 36241 | tgctggacct ctgcaatgga aatgtaaagg tatcctatac tttggtcttg ccttaaaatg |
| 36301 | ttcaaacggt gacatacata attattgatg gaaccatcta actcaagcgt ggtgcagcat |
| 36361 | ggatgtcttt ctgaaaggat gtccaatggg aaggctaaca ttttaaaaag ttccattgag |
| 36421 | gctaaatgtt ttatgtggat tatcttgttt aattctcact aaaagtctgt aacaagggcc |
| 36481 | ctatattctg ccattttagg aaacagagga atagagagga tagctaactt acacaaggct |
| 36541 | gcacagatag ctgtgtctaa gaatttgagt caggctgaat actgtctgtt ctattaaaga |
| 36601 | atgagatagt cctgtgtgac attgtcaaga gaagtatcta gatgaaaatg aggggaaaac |
| 36661 | cagaattctg caaatgaagg agaggatggg agctgaaaaa aaatagacag gaggtattga |
| 36721 | caaatttttt gataaatttt tcagagaaaa gggaggagtc aaaaaaggaa gcagtagctg |
| 36781 | gagctatgtt tggtgtgtag atcacgttgc attgcaagat ttctcttcga aaaatattga |
| 36841 | aatgtaaaaa aatttaaatg ttaaaatatt aatgacactg agctcagaga gaagaaaaat |
| 36901 | ttgaacatac ataagagaac aaaatactca atgaaatgtg gtgctggata atgcacaagg |
| 36961 | gttgtatttg ggggccaagt agaatgataa actgtataca gagggtgacc acatgttctg |
| 37021 | gttttcccag gacagttctg gtttacacct gttgtcctgg aatcattatt aatagagtct |
| 37081 | ctcttttact ttcagaagta tccaagcttc cacgataaat tatatggtta ctctccttag |
| 37141 | acaggaagac agaatgtcct tgtaatagga agaaaagaga agagggaagt tttcagtata |
| 37201 | aatcagttca taagtatgat ggatggaatt tgagaaaatt ctgttcttat ggtctctatt |
| 37261 | tcctctagga aggagaaatt agggttatct tttgaaaaca cttttgttaa agggtcaggg |
| 37321 | aagggggtga ggatgaagtg agttctaaaa tagctgtgga atgggagaga tgactcacaa |
| 37381 | agtaaaccag gattgtaggg cagcattaaa gttttagtag agagcaggac cgtgaatatg |
| 37441 | gagtagcatc aatccacatg gttgcagggt ttttccctcc agtgttgctc tgctgtacag |
| 37501 | gtgtaagttc agaaaatagc agaaagctaa attcaaaggc aagacataaa tgttggagtt |
| 37561 | ttgtcaggaa ggaaaatagg gcaaaggatt tgagactatt gcaaaagaga gattgaagca |
| 37621 | tgaatcatgg aatctttagg taccaaatac acctataatt tctctttctt ttcttttctt |
| 37681 | tctttccgtc tttcttgttt gtttgttttg ttttgtgaca aagtctcact ctgttgctca |
| 37741 | ggctggagtg caatggcaca atcatagctt actatagcct caaactccta ggcttaagtg |
| 37801 | attctcccac ctcagcttct tgattagctg gggctacagg tgtgcactac tgtgctcagc |
| 37861 | tatttatttt atttatttta ttttattttt tttactttac tttattttat tttattttat |
| 37921 | tttattttat tttattttat tttattttat tttattttat tttattttat tttattttat |
| 37981 | tttattttat tttattttat tttattttat tttattttat tttattttat tttattttat |
| 38041 | tttattttat tttattttat tttattttat tttattttat tttattttat tttattttat |
| 38101 | tttattttat tttattttat tttattttat tttatttcat agagacaggg tcttgctatg |
| 38161 | ttgcccaggc tggtctcaaa ctcctcaagc aatcctccag ccttgacctc ccaaaatgtt |
| 38221 | gggattatag gcatgagcta ccatgcccag ccatacctat gatttaatat ccaaatatga |
| 38281 | aatatcctcg gatatgagcc taggattaaa cactatagct ccagcaatat aacagacaag |
| 38341 | ataatatgaa aatcttccaa ctagcccgga aatgctgggc tagttgtata actgataaga |
| 38401 | ataaaatata aatctctgga agctcaaaat ggacaggaac tgcaaaaacc ataagctcaa |
| 38461 | tgagctggtt tcttattgtt ctaaaggtaa tgggacaggt gctcatctcc ataacctagg |
| 38521 | agattggttt taagaccctt aaaggggcag gagacagggt cttggcccat gtgaaatgga |
| 38581 | ggttagcatt caaaaccttg tataaagctg agaaccacaa aagtagaaag atgggccaga |
| 38641 | aattccatct tcaggcacaa aaacacaaca caatggcttg tctgtcttta gtcgtgaggg |
| 38701 | agaaaaaacc aggtcatctt aggagtgaat attcaacact caggactata gcttccatag |
| 38761 | gcttgatatt gacttaatac catcagcaca gtctgggaat tctcagctgg aaaaatgaac |
| 38821 | atacaaactg gtttcaggct gggcacagtg gctcacccct gtaatcccag cacattggga |
| 38881 | ggctgatggc ttgagcactg gagaccaacc tgggcgacat ggcaaaactc catctctaca |
| 38941 | aaaaatataa aaattagcca ggtgtggtgg tacatgcctc tagtctcaac tatttgggag |
| 39001 | cctgaagtgg gaggatggct tgaacctggg aggtggaggc tgcagtaagc caagattgtg |
| 39061 | ccactgcact tcagcctggg caacagagtg agaccctgtc tcaaaataaa aaaataataa |
| 39121 | taaaaaacct acatacacat aaaacattgg ttccaggcca aaaaacccac acacacacaa |
| 39181 | aacattggtt ccaggccaaa aatttaacaa gaaaaaaaag ctaaaagaag aagcacttta |
| 39241 | cccaagttac ataggctttt catggatata cctacactga ggatgagttc acaatgcaaa |
| 39301 | attgtcacac ctacagaaaa caacctacct tgagtgagtg ttgtccaaca taatatacaa |
| 39361 | agaaaattca gttaaccaca taacatcata gaaattataa aataagtatg cttaaagtaa |
| 39421 | ttaaaaatgc aaaaggagga ataaggaaaa caaaaataca ttagggccag gcacagtgac |
| 39481 | tcacgcctgt aatcccagaa ctttgggagg ccaaggcagg cagattactt gaggtcagga |
| 39541 | gttcaagacc agcctggcca acattgtgaa accctgtctc tatacaaaaa ttagccgggc |
| 39601 | gtggtggtgc acgcctgtag ttccagctac tcagaaggct gagacaaaag gatctcttaa |
| 39661 | acccaggagg cagaggttgc agtgagccaa gattgcacca ctacactcca gcctgggcaa |
| 39721 | cagagtgaga ctttgtctaa aaaaaaaaat taaataaata aataaattag gaagacaggc |
| 39781 | ggttttgata aataattaaa cacaatttta acacataaaa atataattaa aattaaaatt |
| 39841 | caatggatga gtaatagtat attagagata aagagataat tcagccatta gaaatcagct |
| 39901 | cttcagaatt tagaattcag tgcagaaaga ttttgaaagc atagaaaaga tgaaattgaa |
| 39961 | tttaagaaat gataaaagat acattacaga aagagaaaat acagaaaata tgagaggcaa |
| 40021 | tactcaaaaa catgatgact aagaactttc cagcattgat gaaagactta aatcttcaag |
| 40081 | tacgtgaagc ataccaagtc ccaatcggta aaaatgaaga gcagagaaat aagcatttca |
| 40141 | cacccagtca aatcataagg aaacatcaaa gacaaagaga ttatcttaaa atattgaaga |
| 40201 | cagattattt ataagggaca acaattagga taacaataga gtgtggggaa actatcactt |
| 40261 | tcatctatcc ttacaatttg gtatagccgc atgaaggttt atgaaaaata aaaatcaaac |
| 40321 | tttaaaatgt atatagattt gtctcagcaa tttaacttct gacaatttat ctaaaagaat |
| 40381 | atttgcacaa gtgtatgttc aagattattt gttacaaagt tacttagtaa aaagttgtaa |
| 40441 | agaacttaaa tttgcttcaa taggacacca tctaaatatg tgatgtaatt ccatgcagcc |
| 40501 | attaaaaaga atgagatgga tttatataca ttaacttcta aaattatcta ccgtatatgt |
| 40561 | gacttgggtc tgctttccat ctctgagcct cagcttcctc ttctctaaaa tagagataaa |
| 40621 | agcagaacct acctcttagg atgcttgatt attagttatt atagacacat agtgtgtact |
| 40681 | atataattat ttgctgtttt attatttatt tcatgtattt aataagtact tgtatagtgc |
| 40741 | ttaccatgtg caaggtactg ttctaagtgc tttagaatat taattcatta tcaatataat |
| 40801 | atgttcataa gtgaaaaagt aagtaaagaa cagtatatct tattattata tattagagta |
| 40861 | catgtatatc gaactcttaa gtcatcatct ctgcatagag aattatatgg ggggtcccgg |
| 40921 | cacgatggct cacgcctata atcccagcac tttgggaggc cgaggtgggc gaatcacgag |
| 40981 | gttaggagtt tgagaccagc ctggccaaca tggtgaaacc ccatctctac taaaaataca |
| 41041 | aaaaattatc tgggcatggt ggcaggcacc tgaaattcta gctactcagg aggctgaggc |
| 41101 | aggaaaatca cttgaaccca ggaggcagag gttgcagtga gcagagattg tgccactgca |
| 41161 | ctccagccca ggcaacagtg caagattcca tctcaaaaaa aaaaaaaaga aagaaagaaa |
| 41221 | attatatggg gggtaacttc tattttatac tttatgtagg cctatacttt ttgcatcttt |
| 41281 | ataaagagaa ctttataatt aggaagaatt aatacatgta tttatgcttt ggaaaaaaaa |
| 41341 | cacttcttcc aaatattata ctatttttga taaagtaaca tgaatctttc tgttcattag |
| 41401 | tttgatattt ttgcagttct gttgctccat tgatctccaa gacaaactca gggatgggaa |
| 41461 | aagttttgtc aaaggtaatt gattcctatg ttgcttaggt tttcacaact cttggatatt |
| 41521 | ggctgaagaa ttttgaaggt ggaggttctg atgaacagga catagaacat gccatttctt |
| 41581 | tagggaagga atcaatccat aaagctgcta agcttgagac ctacaagagg aagctggcag |
| 41641 | agtcacctag atggaggaag tgaagggggt cagagaacag cagctgctgc tgaagtctcc |
| 41701 | actcatctga ctcttctttt ctatgtatag atggaaatct gaaatttatg attttatagt |
| 41761 | ctggaatagt ttgcaggatc cagcacaaaa gttttgttag ggttctgatt tctcagtgaa |
| 41821 | cattttccac actttgttgg gtgttactga cttacagcat aaagggaaaa gtaaaacaaa |
| 41881 | gggaagttcc tataggaaag ggaagcaccg ctaggtaaga actatttagg tatactgatt |
| 41941 | tgacattgac agggaagcct cagtcatctg ggaggcagaa gaaagttttc agtctcacaa |
| 42001 | ctgtggaggc tcttctggaa aggttaagcc ctgtctcagg cccccatgtg ccaaagggct |
| 42061 | ctaggagagg attcacggca ggatgtgacc agggaacatc ttattgtctg atgtcaccag |
| 42121 | agaaatctac atatcatatt tcatacattc taagatgctc attatttcat attttggcat |
| 42181 | atctgggtta catctcatca ttaatccctt acaatcacaa ttgaataaat tttagtttct |
| 42241 | ttgttgcaca ttttaatatc tctgaaattg agatttttct tacaggtgct aaaggcattc |
| 42301 | acacccagtt tgtggcttct gccctccagc taagatcgtt ccttagctgt ccccaaatat |
| 42361 | gagaatgaat atacaatgat aaaacaaaaa taaatcaacc atattggctc ataaagcttt |
| 42421 | ctttattttt aactagaaat tttccttctg cttgagatca tttcatttgt tcctcacaga |
| 42481 | aatgaaagca ttattttcag tgagatgatg ccacctagcc tttttccctc aaaacctttt |
| 42541 | atttgctgcc tattcatttt attaggttga aatagagact aagtggcttt tcagggccat |
| 42601 | atagaggaaa ttatctttta agagacaatt ttgtctgctt ctctaatcca cttcacattc |
| 42661 | tactttaggt atggtgtgta gccaaagagt gatctatgtt gtttttctta tatatctgaa |
| 42721 | accattttct cctgagtacc tctaaaggac aggaagacag atactcaggg gcagagggat |
| 42781 | atggcagtgg aacatgctga tttgaatggc agggctggaa ttttcagtga gggtttgcta |
| 42841 | tcatgattgc ttgggacaac ttttgaaatt ctgattttat ttccctgttt tgaccagtgg |
| 42901 | catatgcccc ctttaaaacg ctagaccaga gggccaacca tagtggctca cacctgtcat |
| 42961 | ctcggcactt tgggaggtcg aggcaggcgg atcacttgag gtcaggagtt agagaccagc |
| 43021 | ctgggcaaca tggtgaagcc acgtctctac taaaaataca aaaattagcc gggcatggtg |
| 43081 | gtgcacacct gtaatcccag ctactcagga ggctgaggca ggagaactgc ttgagcctgg |
| 43141 | gaggtggagg ttgcagtgag ctgagatgga gccactgaac tccagcctgg gcaacagatt |
| 43201 | gagaccctgt ctcaaaaaaa tagagagaga gagagagaaa caaataaaga gagagagaga |
| 43261 | gagagaatta actcctctga ccagtgcagg ggctcatgcc tgtaatccca atacattgga |
| 43321 | aggccaaggt aggtgaacct cttgaatcca ggagttcgag acaagcctgg gtgacatggc |
| 43381 | aaaaacctca tctctacact gaagtaaaat aaagtaaaat aaaaataaaa taccagacca |
| 43441 | tacaaatcaa atcttttaag gtgataacat aattactgtt ttttatagct ggggggtgtc |
| 43501 | tttgttataa gattttttaa acattgcaaa agactcattt aaaatttata aatatctaat |
| 43561 | tttctttttt cccctcacat tctctgcctt aactaagatt ttttaaaatg cacttattta |
| 43621 | tttgaggctg aatgtggctc ttactacatt ctgtgaatat cacattcaga tgaatactgc |
| 43681 | tttgtatata gaaactggct gaatgtagtg tacttgagac ccaaattaaa atagatggat |
| 43741 | gctggagtgt atcaagctct tgttaattgc attttctatt attatcttgt ttacccttga |
| 43801 | agctttaagc cattaaggaa aatattgggc agaagctgga atcaagagag ggctgtcagt |
| 43861 | cagcaattgc tggtgggaag aggtgtcttg gcaccaatac aaaactgcaa ataagagcca |
| 43921 | gggagggtct agaagcagaa caaaaagaag agagtccttc tgggtacagg gttattgtag |
| 43981 | ggctccttca tgtttaaaca actccttatt tcctcagggc tttgaaaagc agcctcttgt |
| 44041 | caaaacgtgg agaggcattt tggggagtag caagggaagg caagtcatat ggagaattta |
| 44101 | agtggcttca gtgcatgagt ctccatcact tgtcaggaca cctgcgtgct gccagcccag |
| 44161 | tttctttgca atctgaaagg ctgcactatc tacccttaag gccaacaaat ctattcagaa |
| 44221 | actttttatc tgaagatatt tttgcagaca tatgaaatag gtaagacaag tagaattcca |
| 44281 | aaagatgaaa ataaataatt cctatctatc tatctatcta tctatctatc tatctatcta |
| 44341 | tctatctatc atgtcacaga agcagatgtc aatagacata actataaaag atcagcattg |
| 44401 | tcttcaaaat tttagacttc tggctgcctt ctttcctaga gatcaaattt acttgttgca |
| 44461 | tctacaaaag tggaagcaat catgtaatgt ttataccaca tttgatttgc tttttccttt |
| 44521 | gtttatgctg cagctcttaa tatcacaaat gccacagaga gaggtattta aaaccccacc |
| 44581 | gtgtgctgtg ctctcagtga ttgcagtttt aataaaattt gcagtggtcc tgttcccggc |
| 44641 | atcagtccta attgtttgat gccactaaca ggtttgattt gctgtcatga gtacattgtg |
| 44701 | catttgaaaa ccaaacaaag aaaaaaatca caaacctctg atatccctgg aggctatcat |
| 44761 | tgggaaaatt aaatctgcat gatttaatag gatgcatgtt ctaaaagagg agcttgaccc |
| 44821 | attgattttg actttagggg atattatggt atattatggt atggtttcat ctaaaaagtt |
| 44881 | ctataaggct ttcagagaag aatggtaaag tggtccttta gttgaacagt tgtcactgca |
| 44941 | tttctctaag aaaaagtcac ttgtataata atgatagcac tgaatagata atacataatg |
| 45001 | cttcaagtca acacatagtt aggccctctt aaagtgcagt gttaaagaaa atgtacttta |
| 45061 | aagtaattaa attgtttagt ataatgttga cattatatta tttgaaatga aaagttatcg |
| 45121 | tgacctgaaa tacatatact ctaattggag tgagttttta atatcagttt tcagatatta |
| 45181 | aaatgagatg cataagttca agcatacata cacagtaaag gaaaattgct ttctattcaa |
| 45241 | tgcattagtt catccaacaa agacacactg agcaatgttc caggtgcgac atgagacaga |
| 45301 | gaaggccatg cctccactgg gcttaatttc aaaatgtgac tattttgatt catgcaaaga |
| 45361 | tatcatggaa agctattaga attctatggc aagaaggagt tcaagtaatt ttatattcag |
| 45421 | ttattttctt cagatatcaa atacatgact ctgagactgt aattgttata tttatttccg |
| 45481 | tgaacaacaa actatacaag gcattatttt tggagaggac ttaatatagt ggttaataac |
| 45541 | atgggtggta ggatctcaag aaaactgcat gggctctaga gtctcaggta aacttcttga |
| 45601 | gtccaaatcc tggttctacc acctatcaca tgtgtaactt ccagtaggtt acttatctct |
| 45661 | ttgtgcttca gtttccctac ttgtaaaatt aggttaataa tactatctac ttcataaatt |
| 45721 | gaagaatgta gaagtagatg atatatttat agctcttaca agagtgactg acacagagta |
| 45781 | aacactcaaa tagaaattat caatgttagt tctagggcta atataacttc tgtgcaacag |
| 45841 | aagcagtgat ttacgcactt gtgcagacaa ttatagctcc cttttccatg tgtcatcttt |
| 45901 | atctctgagg tacaagcaat aaagtccaat ggcaaaaact aaatttttat ttttaatagt |
| 45961 | ttatttgtat taattttggt tctagcaaaa gaataataac caactttaaa tctgcaagga |
| 46021 | gaccccaaag tcatatagtt caattcctta gtttgtagat aaagaaattt gagactaggg |
| 46081 | gcagtggctc acgcctgtaa tcccagccct ttgggaggct gaggcaggag gattgcttga |
| 46141 | ggccaggagt tcgaggccag cctgggaaac atagtgagac ccccctctct aaaaaaagta |
| 46201 | tttatttaaa aatatttaaa aaaaataaat ttgagaccgg aaacatcttt tattttatag |
| 46261 | ataaagacac ttgagaccaa acaaattgtg actttgccca ggttggcaaa ggaacttgag |
| 46321 | tgtcttattc ctagtctagg acatgttcca ctacctattc cgattctttc ctctttttaa |
| 46381 | tatttatgtt gtgtagataa tactctgcag agttcacatg ctttagtatc agacagacct |
| 46441 | gggtgttaat cctgactgtg ctgcttacca gttatgtgag gtaggataat ttacttgacc |
| 46501 | tctgcaagtc tatttcctca tttgtgaaat ggggggtaat attatctaga acacagagtt |
| 46561 | cttaaaatgg actcagatag catattcaca gtgtctatgc agtagctctt tgataagtat |
| 46621 | ttgctgttgt tggtattgtt gtctgttgtt tacattgtta taaagctgga tgacatctat |
| 46681 | ccagagaata ccactagtca agcttcttga atttcataaa tagaatgtca acattaacaa |
| 46741 | ctgctcatct ggtctccaaa gaacataaat ctactttcaa tcagcaatgc tttgtacctg |
| 46801 | cattcttgtt acttgttaga gattataact ttggtgtggc ttcaccgtga caagtgataa |
| 46861 | agggaagctt atctaagaag ctgtgtaaag aggtgccttt aatggctaac aggagagagg |
| 46921 | ctatttggag tcaacctgtt aaaaaaaaaa atagtacaac aacataaaag tgaaggcggc |
| 46981 | cttcgtgtgt atcctgagct tatatcatat tttattcact atagaaatag atatttgcca |
| 47041 | aaaatcttgt tgaaaatact gatccattct agcactcact gctcccaagt aaatcccaag |
| 47101 | tgagaaatca ctttaaaaat ttagtgccat aaaaaatgat gagttcatgt cctttgtagg |
| 47161 | gacatagatg aagctggaaa ccatcattct cagcaaacta ttgcaaggac aaaaaaccaa |
| 47221 | acaccgcatg ttctcactca taggtgggaa ttgaacaatg agaacacttg gacacaggaa |
| 47281 | gggggacatc acacaccgga gcctgttgtg gggtcggggg agaggggagg gatagatagc |
| 47341 | attaggagat atacctaatg taaatgatga gttaatgggt gcagcacacc aacatggcac |
| 47401 | atgtatacat atgtaacaaa cctgcacgtt gtgcacaggt accctagaac ttaaagtata |
| 47461 | ataataataa aaaaaattta gtgcctggct gaagctctgt gaacaatggc tgcagaggag |
| 47521 | cttcctgcca gcctggggaa gccatagcct gcacatgatc tgtccctttt agtgaactaa |
| 47581 | ggttgacttg atggagccaa tgctggcaaa ggtctctgag gaaaactgac ctggatttat |
| 47641 | acaaggtcca gccttgcagg ttgtaaggga ggtcactttc atggcaggcc aagatccttg |
| 47701 | agatgttttg aaaatttgaa ggagaaagga attaggccaa aatgaataga tactgcagac |
| 47761 | gaaatcttat ggagaaattt ccttggactt acatccttag cctcagcaca gcaaattaat |
| 47821 | agaagcttgc agaagaccaa gctgagattt cttatagaga cctccagtgg aacaaactta |
| 47881 | aagaaggccc atatggttaa cgttcttgat gcccatatgg ttaacattgt tgatgcacct |
| 47941 | gtgtaaagaa ccaggctaaa tctaataagg ccatactcac tttgtcgtca agaatctttg |
| 48001 | agattctttt taatcaaaag tggggagact ataaggaaca atttgtgctt aaatggaaaa |
| 48061 | ttgtgtgttg tcattctaga gcacacctgt gttgtgtgat tccaattctg tgggagctgt |
| 48121 | tttaaagctt tataaattgt agataactgg cagcaatgct aaatcgttct cctccataga |
| 48181 | cttgcaaatg aattctgtcc agtggaaaag caaccaaaaa acccaatctg gctctgtttg |
| 48241 | cacagtcatt ttaatattcc tgatctataa atgggtctgt ccttgggatt ctcttttcac |
| 48301 | ctggttgcaa cacttcactg gtttccttgt gaatgagtta aataaaacct ttaactcatg |
| 48361 | tttattctga aaaaaaaaaa aaaaatcagt gcctgggttg gggagaaggg tctctgctga |
| 48421 | gaaactgaca ttttatccat tcctatagta agtactgtgt actttatttt cttttacttt |
| 48481 | attttttgct cggcctggca tgtatcttag aatccaattt gaagcttagt catagtatct |
| 48541 | gttacaccaa accatccaga ggttttctcc ctgatcctaa tatattgtta ttcatttaat |
| 48601 | ttatggccat atcaatatac cttccttaag atctctatgg aaagagaaag aaataactaa |
| 48661 | gttgacggat tcatggaact tctcctccaa gtggttctca aatgccagtg cacatcagaa |
| 48721 | gcatctggag atcttactaa atgacacagc ttcaggatgc attccccagt tattcatatt |
| 48781 | tgataggttt atgttagtgc tttttaacaa caccccaaag gatccttaat gcagtgaccc |
| 48841 | atgaaccaca ctttgaaaaa cactgacttc aggttttcta cttagaggga ctttattcca |
| 48901 | gatgaatcta aacaattcaa ggagaaagct tatctgcaaa tagtaagata tcaatgttag |
| 48961 | gatagcttca aattcatcca tttgtggaat gcctactatg ttctatgcaa tgaggtaatt |
| 49021 | cttaaggata cagacgaaag agagatgtgg ttcatttctt gaaaagtgtt caaattcagt |
| 49081 | gaggaagatg gatatgcaaa caagtaaaat gcaatatatt tacaatgata gaagtttgtg |
| 49141 | tagggttata tacacaggac agaggagata gggaaaggtc agggaaatct tcagagaggt |
| 49201 | gaaggccaac agggctttga ttgatgaaga gtgaggagct cttggtgagg atcatatcac |
| 49261 | attgagggag gaaaaagaaa atagaggaaa aaattcagat ttaccatgca agcacagcct |
| 49321 | gacactgact cataaccagg gagtaaaaga aggattagag agtttgggac agtatctatt |
| 49381 | agtggtgcta taaaaacaga ataaaccatg atttgactca caggctaaca tggagatcat |
| 49441 | cttcttggta tcttctcttg gacctttttt tctttttttt tttttttgag acagagtctc |
| 49501 | tctctgtcat tcaggctgaa gtgcagtggt gcaatctctg ctcactgcaa cctccgcctc |
| 49561 | ccaggttcaa gcgattctta tgcttcagcc tcctgagtag ctaggattac aggcatgtgc |
| 49621 | caccatgccc agctaattct tgtattttta gtggagatgg ggcttcacca tgttggccag |
| 49681 | tctgctctca agctcctggc ctcaagtgat ccacctgcct cagtcttcca aagtgctagg |
| 49741 | attacaggtg tgagccacca tgcctagccc tctcttagac ataattttaa acgggtagat |
| 49801 | acctaacatc ctgacaatgt gggaacctat ggtttggagg catatggcct tatctggcat |
| 49861 | ccagaattcc atctaaaagg ttacagaaat gttcccaacc caagccactt taggcaactg |
| 49921 | ggatactgtg gtatgattta agattaagtt tatggcttct atttattttt ccaactaaaa |
| 49981 | tttccggttg gctaaaacaa aaatcttatt cttaatagac ctcagctcgt ttaacgcatg |
| 50041 | tcagagtgca ggcatcttcc agagcttaga tcaaaaaagt tatttaaaag taaactaaga |
| 50101 | ttttgataaa cgtaccatgg ttatacagga tgctaacatt aggggaagtt gggtgaacag |
| 50161 | tacatggaaa ccttctatac tatctttgca actcttatgt aaatctaaaa ttatttcaaa |
| 50221 | ataaaaatgt tttaaaaagg taaactaaga aatacttctt tgtcctggta attaagcagt |
| 50281 | ttagcatctc ctcccatcaa aaacagaaaa tgaactttct ttaagaagaa aaaagtgaat |
| 50341 | caggaatgaa gtcacatatt ttattggctg atgattaaac ttctaatata ttttcagaac |
| 50401 | cagtttaaca aaagtgcggt tgctttatat acacctaaga ctgaaacaaa aacaagtaaa |
| 50461 | ctttgtgtag ctcttgtctg atttacatgg taaacatttc ttgctcgtac ccactattca |
| 50521 | ccatccatat ttacaagtga gaaaaacgtt accatctcaa aggccacaga aatgttccca |
| 50581 | acccaagcca tgggagggaa tcccacatca ttgaggcagg cagcccttat tttgtcctgg |
| 50641 | agaagagagt cactgtattt gaggatgatg tgtatgtttt ctaaaggagc ttgccttctg |
| 50701 | actccttcag agttggagcc acagtgggta aacctcttca ggcattgccc tgcttcaaag |
| 50761 | cagacagaaa cgtaaacagt ggcagtacac caggatcggg tttctttgct ttagctacca |
| 50821 | agagtcagga aagagaaacc aggtatttat ttaggtttgt cattattgct aggttttctt |
| 50881 | ttaatgataa gggtttgaga aaagaagtat ctgttcagaa acaaagtatc acagagcctc |
| 50941 | aataggatta ctttttcaac cacccagggc cacgtgtttt ttaagtcaaa tataaaaaca |
| 51001 | atttaaccca ttttgttctt tgtagtcctt gccttctaac aggcatctcc aactcagtta |
| 51061 | ttcttaaaat atgatgctag atgagacaaa tagtctattt aagagcagat tcaaataatc |
| 51121 | tgtatagatg agcatctatg ttttaataac agggtcgaaa gaatttctct ctctacagag |
| 51181 | tgatttctct gtctcaaata attcagttta tgaaaggagt taaggatatg ctttttaaca |
| 51241 | ccggaaaagg gtacttaaca tgatcaatat ctaggataca gccagctaaa gcagattgac |
| 51301 | gttacaaaat aaaataaagc cttctccctt tgaagtccgc taaaggaaca gctttcatga |
| 51361 | ctcagaaaga tatcctagag atcaaaaaag ctctggagaa agaaaaagaa ctagaagacc |
| 51421 | tggcttcaaa atttacctct agtacttccc agttatgagt tagctgctat attaattaat |
| 51481 | tgctttatat aaatcatgtc atttaatctt ctccacaatt ctatgagttg aaagttaata |
| 51541 | actacgtttt ctggataaaa aacctgaagt tcagagaagt tgaacagcaa ctcagaaaaa |
| 51601 | acagctagtg agagtgaagt gaggatccaa ttctaccacc tggattgctg ttagggtatt |
| 51661 | gtacatgagt gtaaggaaag tatcctacta ctaagatttt gacttcccaa cctctgagtt |
| 51721 | tgtcctttga aaaagccctg caatggtgtt cttatttacc aaatggtcag agattttctc |
| 51781 | aagatattga ctataaggaa aatgataaac aacttaatga aatggcacta tgaaaataaa |
| 51841 | ataatctagg aacaaatatt attttatttt catagtgcca tttcattaaa ttgcagcact |
| 51901 | attcacaata gcaaagactt ggaaccaacc taaaagccca tcaatgatag actggataaa |
| 51961 | gaaaatgtgg tacatatatg ccatggaata ctatgcagcc ataaaaagga aagagatcat |
| 52021 | gccctttgaa gggacataga tggagctgga agccattatc ctcagcaaac taacacagga |
| 52081 | acacaaaacc aaacaccaca tgttgtcact tataagtggg agctgaacga tgagaacaca |
| 52141 | tggacacatg gggggaacaa tacacactgg ggcctgttgg aggctgggag tgggaggaag |
| 52201 | gagagcttca ggaagagtag ctaacggatg ctaggcttat tacctaagtg atgggctgat |
| 52261 | ctgtgcagca aaccaccgtg gcacacgttt acctatgtaa caaacctgca catcctgcac |
| 52321 | ttgtacccct gaacttaaaa tagaagttgg gggaaaaaaa agaaaaaaga aaatattagc |
| 52381 | ttggtgcaaa agaaataatg gcaaaaactg caattacttt ttttgcacta acctaataaa |
| 52441 | gtgatcttca gaaaacagtc tactttttta aaaggcaaaa aacagagtgg tttatataat |
| 52501 | tggaataatt tttacatctc tttttgatac acacttctta ttttctaaaa ttttcacaaa |
| 52561 | tcatcttaaa cattaatgct gtcaccaaat aaattctcac aaagtaatag aaagctcagg |
| 52621 | aaattaatat ttgagaagta aatttagagg caaggataac ttaaactgag aagcatttat |
| 52681 | aattttccat tttgagaaat actctgtagc atttataaac attatatttt aattttctat |
| 52741 | ttcaataaat taatattgta ccagaatacc aaactgcatt tggaatcata ggcaactgtt |
| 52801 | atgtttgtcc tttcttgata tttgaaaaaa atgaccttcc ttttgaatgc tgccctcata |
| 52861 | atttaaaatt ggccttctcc ttcacagggg acattcacga taaactctca ataggcttta |
| 52921 | atttggagaa ttttagtttt gccttagcag cttttcttat atcttctctt aattctataa |
| 52981 | gtttaggctt tatatgtttt gaaattatat tgttaagtgc atacacatta attattgttg |
| 53041 | taattcctcc tctgcagatt gcatttttaa cctttggatg atgttcctct tcatcccttt |
| 53101 | tttataatac cttttagcat aattctgtat tgtttgatat taatatcatt accacatttt |
| 53161 | tcctttgttt tgtgttttcc tgtgctcttt gtttggtatt cttctatccc tttcttccaa |
| 53221 | cctccccaca atcttgtaag cagaatatag ataaatactt ttgtaattca atccaaaaga |
| 53281 | ctttattttt agtagataag tgtaggtctt tgtatttatt ctgaaaattt atttctatta |
| 53341 | ttctttagac gatttctatc atgtattatg ctgtttcttt tctctgcttc tttttttaat |
| 53401 | tccttttctg ttttctggta gatcattata tattttttta attctccact ttaacccaca |
| 53461 | ccacaaccac tttgctgatt ttgaaattgt ggatcacaat gttatatttc agtgattact |
| 53521 | tttacatttt aatatacata tatgatgcta taattctaac agtttaagat catgtaggat |
| 53581 | cttctccagt ctctttaact aaagcaagca tcttatcatt ctttaaaatc ttatctaaca |
| 53641 | gcaccttccc tcatcctctg tcactttcct tggcattatc caaaatttta tttatcttaa |
| 53701 | aacactgaat tattttatgg tccataaata gttctatcaa tatatgccac caattgctgt |
| 53761 | gttcatcatt caattttcat gtgccttttt tcttctgttt ccaatttctt atttctgaaa |
| 53821 | catattattc attagttcat tcaattagat ctgtgagcag atatctttgt ctttgtcttt |
| 53881 | gtatgtttga aaatgccact attttaccct gatcattcag gtgaacattg aaatctaagt |
| 53941 | tgacagatat tttccctcag tgttgtccta tgtcttctta catcaattga tgagaagtgt |
| 54001 | tctgtcattg taactataat tctttggtag gtaacagagt tcttctatct ggtcttttta |
| 54061 | cctttgatta ccagtacttt taccataatt tgtcatagaa tctgtattag ttatctgttt |
| 54121 | ctctgtaacg aattacacag aaactcaatg ttttaaaaca ataaacgatt atcacttcat |
| 54181 | agtttctata actcaggaag tacagagtga ctttcccagg ccaatgtatg gattatgttt |
| 54241 | gcagtaaaga tgttggccac ttctgcagtc atctgaaagc ttgaatgggc tgaaggatct |
| 54301 | aattcaaatg tggttgactc ataagttggt ataaattgat gctggccagt gcagagcctc |
| 54361 | cctttgtccc catgtgggtc tctcctcagg ctgcttgagt gtccccacac tatggtgcct |
| 54421 | tccttcccta agagtagatg atctaataga gaacaaggag gagttagtaa tgtttttcat |
| 54481 | ggtctagctt caaaagccac acacagtcac ttctgccaca tcctatttgt tagaagtgag |
| 54541 | tctaagtcca gcctacttcc aaggagagca gaggaactat actctacttt tccaagggag |
| 54601 | aatttgtgga caggttttaa aactacaata gtgtcaatgc aaaatgcacc agaaagtctc |
| 54661 | agtaacagaa tcacacaagc agaagaaaga acttcagagc ttgaagacaa ggcttgaact |
| 54721 | aaccaaatcc atcaaagaca aagaaaaaat aatttttaaa aatggacaaa gcctccaaga |
| 54781 | agtttaggac tatgttaaaa gtcaaaacct aagaatagtt ggaaggaaga agagaaagaa |
| 54841 | gataaatcta aaagtctgga aaacatattt gaggaaataa tcaaggaaaa cttccctggc |
| 54901 | ctcgctagag atctagacat ccgaatacaa gaagctcaaa gaacacctgg gaaattcatt |
| 54961 | gcaaaaaaat cattgcctag acacatagtt atcaggttat ctagaatcaa gacaaagaaa |
| 55021 | agaatcttga gagctgtgag gcaaaagcat caggcatctt ataaaggaaa atctatcaga |
| 55081 | gtaacagcag atttctcagc agaaacccta caagctagaa gaaattgggg tcctattttt |
| 55141 | agcctcctaa aacaaagcaa ttatcagcca agaattttgt atccagtaaa actaagcttc |
| 55201 | ataaatgaag gaaagataca gtctttccag acaaatgctg aattagccac cacaaagcca |
| 55261 | gcactacaat ctcaaatgaa ctctaaatct tgaaacagat tctggaaata caccaaaata |
| 55321 | gaacctcttt aaagtttaca tctcacagga cctatataac agtaacacaa tgaaaacaac |
| 55381 | aacaacaaca acaacaaggt attcaggcaa caaatagcac aatgaataga atagtacctc |
| 55441 | tcatctcaat actaacgtgg aatgtaaatg gcctaagtgc tccacttaaa agatacagaa |
| 55501 | tagcagaatg gataagaatt tcaccaacca agtttctact gtcttcaaga gactcaccta |
| 55561 | acacataagg actaacataa acttaaggta aaggggtaga aaaacatatt ccatacaaac |
| 55621 | agacaccaaa agcgagcagg agtcgctatt cttatatcag acaaaacaaa ctatagagca |
| 55681 | atagcagtta agaaagacaa agagggagat tatataatga taaaaacact agttcaacag |
| 55741 | gaaaatatca caatcctaaa tatatataca ccttaacact ggagctccca aatttataaa |
| 55801 | acaattatta ctagacctta aaaataagat agatggcaac acactaacag tgagggactt |
| 55861 | taatactaca ctgatagcac tagacagatc atcaggacag aaagtcaaca aataaacaat |
| 55921 | ggaattaaac tataccctac aacaaatgga cctaacagat atttacagaa cattctaccc |
| 55981 | aacaaatgca gaatatacat tgtaatcatc agcacatgga acattctcca agacagacca |
| 56041 | tatgataggc cacaaaacaa gtctcagtga atttgagaaa atagaaatta tatcaaatat |
| 56101 | tctctcacac cacagtgaaa taaaactgga aatcaactcc aaaaggaacc ctcaaaacca |
| 56161 | tgaaaataca tggaaattaa atgacctgct cctgaatgat tgttgggtca acaatgaagt |
| 56221 | caagatggaa atttaaaaat tatttggact gaacgataat agtaacaaat agtgagacaa |
| 56281 | cctatcaaaa cctctgggat acagcaaaaa cggtactaag aggaaaattc atagcattat |
| 56341 | ctgaaaaagc acatatagac aatccaaggc cacacctcca attctccatg caattggtga |
| 56401 | aacaagaaca atctaaacac aaatgcagaa gaagaaaaga aatataaaag atgagagcag |
| 56461 | aactaaatga aattgaaatg gaaaaaaata caaaagataa atgaaacaaa aagctggttc |
| 56521 | tttgaaaaga taaataaaat cgatagacca ttagctagat taaccaagaa aagaagacag |
| 56581 | aaaattcaaa taagctttat tagaaatgaa atggaaggta ttacaactga tatcacagaa |
| 56641 | atatgaaaca ttattcaagg ctaatatgaa cacctttatg gacataaact agaaaaccta |
| 56701 | gaggagatgg ataaactcct ggaagtatac aaccctccta gattaaacca ggaagatgta |
| 56761 | gaaactctga gcagaccaat aacaagcaga aagattgaaa tggtaatttt aaaattgcca |
| 56821 | agaaaaagca gtccaggacc agacagattc atagctgaat tgtatcagac attcaaagaa |
| 56881 | gaattggtac caatcctatt gacactattc cacaagatag acaaagaggg aatcctccct |
| 56941 | aaattattct atgaaggcag tatcacccta atactaaacc aggaaagagc ataaccaaaa |
| 57001 | aagaaaacta cagaccaata cacctgatga acacagatgc aaaaattctc aacaaagtac |
| 57061 | tagtgaactg aatccaacaa cataccaaaa atataatcta ccatgatcaa gtgggtttca |
| 57121 | taccagggat gcagggatgg tttaacatat gtaactcaat aaatgtgata caccacataa |
| 57181 | acagaatcaa aaacaaaaat cacgatcatc tcagtagaca cagaaaaagc atttgacaaa |
| 57241 | attaagcatc ccattatgat taaaaccctc agcaaaatca gcatagaaaa gacatacctt |
| 57301 | aaggtaataa aagccatcta tgacaaaccc acagccaacg ttatactgaa tggggaaaag |
| 57361 | ctgaaagtgt tacccctgag aactgaaaca agacaaggat gcccactttc accacttcta |
| 57421 | ttcaacatag tactggaagt cctagccaga gcaatcagac aagagaaaga aataaagggc |
| 57481 | atccaaatca gtaaagagga agtcagaatg tcactgtttg ctgatgatat aatcttatac |
| 57541 | ctagaaaacc ctaaagactc atccaaaaaa ctcttagaac tggtagatga attcagcaaa |
| 57601 | gtttcaggat acaaaattaa tgtacacaaa tcagtagttc tcctatacag caacagtgac |
| 57661 | caagctgaaa atcaaatcaa gaactcaact gcttttacaa tagctacaaa aataaaataa |
| 57721 | aatacttaag aatatatcta accaaggagg tgaaagacct ctacaaggaa aactacaaaa |
| 57781 | tactgctgaa agaaatcata gatggcacaa agaaatggaa acacatcccg tgctcataga |
| 57841 | taggtagaat caatattgtg aaaatgacca tactgcctaa agcaatctat aattcaatgc |
| 57901 | aattcccatg gaaataccac gattactctt tacagaacta gaaaaagcaa tcctaaaatt |
| 57961 | catatggaac ctaaaaagag cccacatggc caaagcaaga ctaagcaaaa agaacaaatc |
| 58021 | tggatgcatt accttaccca acttcaaacc atactacaac gccacagaca ccaaaacagc |
| 58081 | atggtactgg tataaaaatg ggcacagaga tcagtggaac tgaattgtga acccagaaat |
| 58141 | gaagccaaat acttacagtt aaccgatctt tgacaaagca aacaaaagta taaagtgggg |
| 58201 | aaaggacacc ctattcaata aatggtgctg ggataattgg caagccacat gtagaagaat |
| 58261 | gaaactggat cctcacctct caccttttac aaaaatcaac tcaagataga tcaaggactt |
| 58321 | aaattgaaga cctggaacca taaaaattct agaagataac atcagaaaaa ctcttctaga |
| 58381 | cattggctta ggcaaagact tcataaccaa gaactaaaag caaatgcaac aaaaacaaag |
| 58441 | aaaaatagat gggacttggc cgggtgcatt ggctcatgcc tgtaatccct gcactttggg |
| 58501 | aggccaaggc tggcagatca cttaaggtca ggagttgaag accagcctgg ccaatatggt |
| 58561 | gaaaccccat ctctactaaa aatacaaaaa ttagctgggt gtggtggtac gtgcctgtag |
| 58621 | tctcagctgc cctggaggct gaggcagagg aatcacttga aactgggagg tggaggttgc |
| 58681 | agtgatccta gatagtgcaa ctgcactcca gcctgggcaa cagagcgaga caccatcatg |
| 58741 | ggaagaaaaa aaaaaacatg ggacttaatt aaactaaaaa gcttctgtac agcaaaagaa |
| 58801 | ataatcagca gagttaacag acaaccccca gagtgggaga aaattttgca aataatggat |
| 58861 | ctgaccaagg agtaatattc agaatctaca aagaactgaa acagcaagaa aaaaaaaatc |
| 58921 | ccatcaaaaa gtgggctaag gacatgaaca gacaatctca aaagaagata tacaaatggc |
| 58981 | caacaagcat ggaaaaatgc tccacatcac taattatcag ggaaatgcaa atcaaaacca |
| 59041 | caacgtgata tcaccttact cctgcaagaa tggctataat aaaaaataat agatgttggc |
| 59101 | atgggatgtg gtgaaaaggg aacactttta tattgttgtt gggaacgtaa actagtacaa |
| 59161 | ccactatgga aaacagtgtg gtgattcttt aaagaactaa aagtagatct accatttgat |
| 59221 | ccagcaatcc cactactagg catgtaccca gaggaaaaga agtgattata caaaaaagat |
| 59281 | acctgcacat gcatgttcat agcagcacaa tttgcaattg caaaaatata gaaccagccc |
| 59341 | aaatgctcat caatcaacca gtggataaag aaaatgtgag acatataaat acacacacat |
| 59401 | acatacatat acacacacac catggaatac tactacatat acatacacac acacacacac |
| 59461 | acacacacac acatacatac catggaatac tacttatcca taaaaaggaa tgaaataatg |
| 59521 | gcattcacag caacctggtt ggaattggag atcattattc taagtgaagt aactcaggaa |
| 59581 | tggaaaacca aacattgtat gttttcatgc ataagtggga gctaagataa tgaggatgca |
| 59641 | aaggcataag aacgacacat tagactttgg ggactcaggg gaaaagagtg gaagggggat |
| 59701 | gagggataaa agactatata ttacgtacag tttaccctgc tcaggtgatg ggtgcaccaa |
| 59761 | aatctcagaa atgaaaaaat tttaaaaact acaacaatgt caaattgttt gtattaatcc |
| 59821 | tcctatgcaa tcccagtgcc acttgaaagg cagtcttcaa gtacttctga aaaatactca |
| 59881 | tccccaaaac acaggcaaaa aattttgatt cagtaaccta aattgtgtgt ctggcattta |
| 59941 | attgtttgtt tgtttgcttg cttaaacttc ataagtgatt caatgaattt cgagttttaa |
| 60001 | aagccaccac tttaactgat ctggggtttc atgtattgct tcaagcctag aaaatctcca |
| 60061 | ccactatgtc ttcaaatata acctctctgc tcttccttct cttctctact gctcacactc |
| 60121 | ctgtaagcat atattagagc tctaaatctg ttcttcccat cttttagtta atctttcaca |
| 60181 | tttctgtcca tctttctctc tggcctcagg gctgtatgat ggcttaagtg ccatcttctg |
| 60241 | attcattaat tcttcacttg accttgtcca agcttgagtt tatcatatct tttgtgtctc |
| 60301 | tgcttgttat tatcaatttc tagcatttct actattatat gtatttcaat tttgcctgtt |
| 60361 | tgttttataa tttattttta tacatatact attatttaag gttcttaaac tccttttgaa |
| 60421 | cttctttgtt ttcttccaga gtaaattaat cccccagtta ctgaattcat taagttgaaa |
| 60481 | gtcaatttat tattgcttag caattgtttt cctaatgtgc tttgaaatta tgatttgcca |
| 60541 | ctgtaaatct tatcatcatc tctctgaaat gatttattcc ttttccagta attttggggg |
| 60601 | ttgccctcta cttccttcat tcatataaga tatttcaggg agcaatttta ggactctatt |
| 60661 | ctgtaatgtt ttgacattca atccagaccc agtcactaaa ccgtcaaaag cataacacat |
| 60721 | gttctgacta cgatactatg actgctttgg tttcaagcag cgagcctcca atcccccatc |
| 60781 | tcatatagta cacttctaga gaaatcaaac ttcaagcccc tttctctgtt tatagaattg |
| 60841 | gaactcagca gtgactactt atctgttgtg ttttgtctac tttcttatac actgagatat |
| 60901 | tcatcttgtt ttttaatatg gctttgtctt tttaggtagc aaaaaattat atcttcatta |
| 60961 | ttgctatgta cttatattct acctagatga ctgaattgta atatcataac accaacttga |
| 61021 | gtagtactct ttgtgctata attattatat atcatcatat aaattctatc cagctgaata |
| 61081 | tataaaaata aaaatttata ttattgtttg aaagaggtca aaagtattga tgtttaaaag |
| 61141 | tgatattttt atccatctag aaaacacaag agtatcaact ccaaagctac tttaactaat |
| 61201 | atgataatta atcctattta caaataaaat gcacttatgt acacatatat taacagtttt |
| 61261 | cctatatagc agaaataacg cattagaaat taattgaaat aatggcattc acagcaacct |
| 61321 | ggttggaatt ggagatcatt attctaagtg aagtaactca ggaatggaaa accaaacatt |
| 61381 | gtatgttttc atgcataagt gggagctaag ataatgagga tgcaaaggca taagaatgac |
| 61441 | acattagact ttggggactc aggggaaaag agtggaaggg ggatgaggga taaaagacta |
| 61501 | catattacgt acagtttcat tcttgttagg tggatagaag tgggaaaagg gtcttataga |
| 61561 | tagtaagtat aaagagattg gttttggttt gattttgatt tacagatatt ctctctcctt |
| 61621 | tacaacacag ttgatcaaaa tggattgcta ctatggtact gagtttctca taaataaaac |
| 61681 | ttttcaagaa aattatggat tactatttac cttggatgtt gaagtgaagt gaataaatta |
| 61741 | tatctcagat tagtccaata agagcagtca ttgattattc taaataccct attggacagt |
| 61801 | tccaacaaaa taggatgaaa atgccccact aatgcaactg gcaaataact tgtttaacaa |
| 61861 | taaatgtgtt cttctttcta caagagactg tgctggactt tagaggaaaa taaagcagca |
| 61921 | acaaaagtct gatactaatc ccagtttaat tctagcccta acctccctaa agaagattac |
| 61981 | aaatcaattt ggaagacaag aacaatgcat tacatataaa gcagttaaat aaaaatttag |
| 62041 | taattaaatg ggaataaaga ctatgaagca gaatatttta aaaatgataa gtggtaaaac |
| 62101 | aataagttca gaagtcaaga gtgatcctag taggctaggc gagcaggata tgtggaaaaa |
| 62161 | ctaggactac tgtttgagct cgaaatgacc aaaaaagtca tacagcgaat aactatacct |
| 62221 | gagcttaaat aggcctatag acttttcttg cataattttc tctaatttaa agtaacctca |
| 62281 | attgtgtgtg catgcatgta aatgtgtgta tgttaatgca taaacatata taaatttgta |
| 62341 | tctataaaga ggtataatat atttgcatta gaaagaaatt ttgatagtct ataactgaaa |
| 62401 | ctataatttc tggtagaact tctaagcttc ctcccaggaa aatgacaaat cttagacatt |
| 62461 | ctttcatgtc ttgagttcat ccactgttta cttacagtgg atgaaatagg tgtattcttt |
| 62521 | caaaattttg cactgcttcc aatcccaaat aaggcaaaat gaacaatagt agcaacaata |
| 62581 | acatcaaact gaaaaatttc aaaggaaaat taatattatc taaaattgag cctacgctca |
| 62641 | aagtagagtt caagtatagt cctgtataga tcatggagtg aaactgaaac gagactcaga |
| 62701 | attgacctgc agttgccctt gaaccctgac aaagaacact ctaagagaag aaaatgacaa |
| 62761 | gtgaatataa ttctcaaaat ttactaatgg aaaaagttta ctgatactct ttaactttag |
| 62821 | tggaataaag ttttggagat accacagttg cagaataaag acacttaata tccctataaa |
| 62881 | aacaccatgg ccatcccaac cactctaagt tacttggctg ataaaggaaa tagaaaaaga |
| 62941 | gagagaggga gagagttttt gttttgtttt gttttgtttg tttgtttttc caaacttaga |
| 63001 | aacaaacaca ttgttctggg gctctagaaa gatgtgtcca gcccgaaggc attcaaatgc |
| 63061 | aatttttcaa gtctaatctg gcccagtcat ggaccttctt tcatttctcc tgattgaggt |
| 63121 | atgtgataca gacagggaat ctgcagtttc ctaatgtcca gatgaccagt tttaaatatg |
| 63181 | aaagaaacta atatttatcc aatacctatg ctttaccagg aaccgttacg aatattgtaa |
| 63241 | aaaaaagatt tcttcatttg aaggtacatt tgacagacga cctctaaatc tccttttaac |
| 63301 | gataaatata ttcaattcag aagttttaaa tagtttttga acttgatgct tataaacaaa |
| 63361 | aaccaagaat gtaccactga gttgatggag gtagggaaag ttcagacagt agaatctaaa |
| 63421 | tgaggaatca caaattcact taaatgtaag gggattgggt gttaagtgta aatatgagac |
| 63481 | cttctcattg ggaagactca atgtctgaaa agggggttga atttctggct ctgcacctgc |
| 63541 | agcaaatctc caagacttag atcctttgtc cggaaaatgg gctaattttg gcggggagga |
| 63601 | agtgaagggt ttcactattt atttatgaca aatattcaac actcacaatt ctctccaatc |
| 63661 | agaagttctt tgagatgctg ttggtcttga ccagtcagag aatggagtca tctgactcac |
| 63721 | ccattctgag aatggtccta cagacactat aggttttaaa ttggccatta aacctgccta |
| 63781 | taaccttgtc aaccttggct aagttcatct ggatggatgc atggtccttg gcacccatga |
| 63841 | tgcggttgct ggcggaacat ttccactgca tgttcaggtc cacaaactca ggctggataa |
| 63901 | tatcaattat gttcaattac agagacatgg ggagagtcaa atgagatcga gtaaaaaatg |
| 63961 | tgcatgaaaa tatccacaat ctgtgaaaga ctatgcaagt gtattattgt cattgaatgt |
| 64021 | tgttagcaat agcatggcca ttttttactt acaaataaat attgcagttg acatattact |
| 64081 | aactctaaga aaggttcaaa tttacatgaa caaataaaac ttcaggaaag aaaactagag |
| 64141 | ttccacttgg ggactacgtc tgacatggtt atattaaaca ttttggaaca attaatttgg |
| 64201 | ttttgttgtc tctcttctag gatttttaaa catttataaa ttctcaattc tttctattca |
| 64261 | atctaaacat tttacaatat cctgtggacc ttacttcctt agtagattcc cagaaggtaa |
| 64321 | tgatttgctc ctttctgctc tccaagcagc ttctgtgttg gtgcagagtt gcagatctct |
| 64381 | gttggtggta ggttctattc tgcagaaacc ccctaatcct ctgtttacca caacatgagt |
| 64441 | ctattgccat agaaatgata gctgagatcc cagattgaac accactgact tcacagcagg |
| 64501 | gcctggtaag aggagactct aagctacaag gggggaacgc aatgtatgag aatagtaggc |
| 64561 | actctgtagc cttgaaagaa agggtaactg tcaacattag aataaaaatt catgaaagag |
| 64621 | ctacattgtt aggtatgctg tagtttattt tctgtataat actgcctggc taaaagtaat |
| 64681 | cattctgaaa tcttatgtca tttaacagtt tgctttcaaa aggtctgcag ttttccttga |
| 64741 | ttgcatttgt ttcttattga aataagaaac aagcagcttc taattaatct tgttgcacaa |
| 64801 | ctgatgtttc tgtggtcaaa gatttccctt ttcatgatgt gttgttttgt ttgcgtgttt |
| 64861 | attgtgtgtg tttgttttgt cttgttttta ttaacagaag aaagtgaacc cgaacaagat |
| 64921 | actaaaggta tttttttttc tttctgcatg gcttcttttt aattgtgtag ctcttttcaa |
| 64981 | ttgtttattt ttgttgacct tataaacagc tctaagtgcc atttttaaag aaataacaaa |
| 65041 | caatgtatgg ggaatattaa gtcacagcac ttaacggtgt tattagatag gcttctttat |
| 65101 | tatataatat gcttactgac catttgcatt tcttaagcaa gattgagtta aaagtaatgt |
| 65161 | ttaaatttgg tgtctctgga aatacaaaca aaatgaaatg aaataaatca gcatgagagt |
| 65221 | ataaatccca catagattgg aaaagaaaga taccctttga ctaaaagact tttggtgggg |
| 65281 | gttggggggc tttacagtct ttggataaat gttaggatgt caaggtgcca atttggtttt |
| 65341 | tattgtgatg atttcaacaa gatcaggagt attatgcaat tgctccaaat caataaaaat |
| 65401 | taagttattt taattgcaca gagacatatg tttgtagtgg aatatgttta gaggaaaata |
| 65461 | gctggaaatt aaatttgtat gggaaaaatt ctactttttc cccaacagtt ttctgtcaac |
| 65521 | agtttctgtt ttctgtaaaa aaaaaaaaaa aaaagaaaaa agaaaaaaaa aaaaggtacc |
| 65581 | ttgcattctg attaagcccc aaactttgtg ctcttctaac ctaaaatttg atatttgggg |
| 65641 | gatgaaattt gtgagttctc atgtaataga gttgactgta ggaattaatc tcaaactggt |
| 65701 | attatttaaa acagtacctc cttatcttag tatctcatat aaagtacaga taatgttttc |
| 65761 | ttatttttaa ataccaggtt gcttacaatt aaatacaggt aaaaaagcat tttacctggt |
| 65821 | cctatgtgaa ttttttttct taatttattc ctatcagtca ttgctggttt gaatcatgta |
| 65881 | acctaaaaac ccttttcaaa ggaagctaga aaataacaaa agaggaaggg aaaataaaat |
| 65941 | ccatgggagc tccttgtgtc ctttgaaata actgctcaga aatagaaatt gataagagac |
| 66001 | ttaagtattc ctctggcact gctcccttac caagaaaaga gaggtagttc atctccagcc |
| 66061 | aaaccactca tttatttcag gacttaacat ggatgccctg tcccttgggc tttttattac |
| 66121 | actccagtgc aatttcagag tgaattatgc ctttatcttt ttactatacc ttactcctta |
| 66181 | agatgacaat caataataaa actgattaca tatctgaaag tgctttgaaa taaggagaga |
| 66241 | gagagaatat aatgtgggta tgttataaaa gattttagaa ttgctggctt cttgagccca |
| 66301 | attttgaata taggattctc cacttgcagt tagcctgcaa gtctgctcaa gagacattaa |
| 66361 | agccccaaac tcatgaatag atcccatgag agaaaaccca gagttaaata tattaccttg |
| 66421 | gtctattttc catgttgcag atgcacttgc ttacccacag gaaaccaagt ggtttagggc |
| 66481 | tcacagtgaa ttggagaagg atggataaga ctatgtacag aaagaggggt ttgatgattt |
| 66541 | ttttgtttaa aaatccatat caaaacagtt ataaatgctt tccaacttac taagtaattt |
| 66601 | aatgttcctt ctattaagtg gtttaattgt ttatgtaaca aaaacacttt tatattcttt |
| 66661 | tatgcagata aaacacaaaa tagggatatt ctatgcccat tttatttaat aagaaacatg |
| 66721 | tcatataact ttttaaattg atgacctaga atgcattttc agattgtgac taccactgaa |
| 66781 | ataatgggat ttactgttga cttgtgtcca agtttgttat tgccggtgta ttattattca |
| 66841 | tggaggttaa tctggcattt atttttaatt gcaactgact ttattgttta caatgcttga |
| 66901 | aaaacatgtc aaaatttccc tcatcacctt tttacctttc atatttgaaa aatggtaaat |
| 66961 | ttgagtatta tgcaattgct cagatagagt tgggtaaaaa catatcagct ttattgctga |
| 67021 | taaggctgga atgaaaaata aagatttcta ttccatgtga ggattagact aacataactt |
| 67081 | atgtttcctc atcctcttcc tccccacccc actctttccc cttgactctt agttgtagga |
| 67141 | tgaagaagcc ctgacctggt aaacttactt acacacatca accctatatc cttcttagat |
| 67201 | gcaagagagc tgggagactg atagctcaga ataggattga gatacacata acatatccaa |
| 67261 | gtcccttccc ccaaccttac cagagaagtc atcatgttcg ttcatttatt tattcgttca |
| 67321 | ctcagcaaat attcattgag tgcctaccat atatatatac catgcagggt tctagaaata |
| 67381 | gagacagata tgtagataga tatatagatt gtatcagtca gctcatgtta aaaagcaaag |
| 67441 | caaaccttgc ccttgtgatg cttacattct agagaggaga gactgacaaa aaacaaacaa |
| 67501 | gcaaacaaat aaatcagtaa ttaccaaatg attcagatag tggtgatatg atacaatata |
| 67561 | aacaagggta aggggaatgg ggatgagaaa gtactatgat aaggaaacat ttgagtagaa |
| 67621 | acccagaaga catctgctat ctcctttgag aagggaggaa gactgggagg aagggtggaa |
| 67681 | actaactttc aatactgaaa aaagggggtt gagtttctat tttatcattt agagagcatg |
| 67741 | tgagagtgca ggagaaagca gcttcccttt aatccctggc cttattgtcc tcttaaccta |
| 67801 | atccactgca cttaatgaaa cccatggtca gccatagcct atctgccccc ttcagggaga |
| 67861 | agtttatcag cagtctcata ttcatgaagc tacctataat gtcagcagta ctcctgtacc |
| 67921 | tgcctgtgcc acactggagc ccccagaaag ttcccatgga gggcagagta ttacattagt |
| 67981 | taaattaatt ccaaagtcac gttgaattga ctctgtttta tttaccatac cacactatgc |
| 68041 | ttagttgtgt tgtgcaatgt ggataatcag aggtttcaca ataagcccaa atgatattcc |
| 68101 | aaatataata acagccaaaa gaaaaaaaac tgctataaat taggacaaaa taaaaccttt |
| 68161 | ccaagaagaa tttaaagtcc ccaaattaaa actcttggct tcataaatta aagcagcaaa |
| 68221 | acataatcga aattacatta ttctccaatt ttttattctg ctcatcttca gccagaaggc |
| 68281 | tttcattcaa aaggcgtttt tgattcaaaa agttcaaggg tatagtcaat gttactagaa |
| 68341 | cagttttcta gcacatgtga ggttgaaatg tgctagattt tccaaatctc tcaaagatcc |
| 68401 | tgcttgtttc agattctcgg gcagtgggat ccatagatgg tcatgcagta gtcccacact |
| 68461 | caaaaaattt aggaaacaca agcatagccc caacccctac aagagttatt ttagccagat |
| 68521 | gatgcacatg gactctaagt ctccattatt gtaaccagaa aacattagaa gagtacctgg |
| 68581 | acgcaccctc actacgatgc ctgtcagtgt tagttgtcaa gagtctgtaa cgtgtgaatt |
| 68641 | tagattaagt tgggagaata agaatccaag cagaggcaca cattgcattt gggtgatatg |
| 68701 | tctcttaagt ctcttttaat ctataatgga tcactctttc ttttttaccc cctaatgcta |
| 68761 | atgattttcc ctgttaaata tctgggtttg attgaattgc atctccatag tgttctttga |
| 68821 | aatgttcctc aaccctttaa tttttgtaga ttgttaattg gaccccagga cttacttaaa |
| 68881 | atcaggctca attttgcttc tgggggacag attcagcttc attttaaaca tttgatagca |
| 68941 | ttctttgtta ttttttaata atctcttttt gccttgaaga cagcaccttc cttgcatttc |
| 69001 | ttttctcctg gttttgggac ccacatttct atatctaaaa tatcaatatc aaaagatagg |
| 69061 | aagtcaaatt aatttaaatc tgcaaaaagc ccccaatagt caaacaaatc aaatgttcct |
| 69121 | taatctcttt aaattatgca atgcttttta agaggtactc aacaatgaga ccaactggaa |
| 69181 | acactactct ggacctgttc tatcattgtc ctgcgtatct ccttatgtag cacatgtggt |
| 69241 | ctcccatact ttagtttaga atccaatatt cacaaatttc aatttaacta gaattaagtt |
| 69301 | tcaaagcatt gtgctaattg gggcttcata ctctaatatt atattctgtg cttaagtcct |
| 69361 | agaaatcaaa aagtaggagc ataaaatata cttacagtat aataggccat ccaaaagatt |
| 69421 | ccgtaatagt ttaaacaaac actgaatttg aagtcagaag acttgagtcc aggtttctca |
| 69481 | ctcatcattc ttgttacctt gggaaaccaa cttacttaaa ttaacaaaga tttggttttc |
| 69541 | actgctataa gataagaaca ggaatctagc caatctctgg agtctcttcc aagtttaatg |
| 69601 | attctgtgaa tctaagtacc ctccaatttg taatgcattg tgagcctcaa tcccactgtt |
| 69661 | tagttaaagg taaaatttct tcctacatca aaataacctc ctcttactta atcagtgggg |
| 69721 | attcagggtc agaaaggaat agaaattact cattctgctg cctacacttt atctaaaata |
| 69781 | tccttttttt tttttgccca atatatcatc tgaaaagttt gacacaattt gtctgccctc |
| 69841 | ccactatgat acagttgagg gtgtaaagtg taattatttg attaattcaa taaaatacta |
| 69901 | tcctgtgctt gctctggtca ggaactctgc ctgaaacaat acagcagtca acgaaaggca |
| 69961 | aggcttttgg cttcaaacaa tctaatggga aaggggaata atagacaagt aaaaaataaa |
| 70021 | taaggtaatt tcagatagtt gtaaggtctg taaagacaat aatgtaggct gatgaaaagt |
| 70081 | gaatgggggg aggattacac ttgatgttgt gtgagtaaga aggacctttc tgagaaaatg |
| 70141 | tcacttaaac agagacttga atgattcaag gaacctacta cttgaggatc tgggggatga |
| 70201 | gtggtctagg cagaggaaga acaagggcaa gtttccatgt taagaacaag ctcagtgtgt |
| 70261 | ccttgagtgg tgagaaatat agggttataa cataaagggc ttggaggctc ggccatggca |
| 70321 | tgtggtatta tccttaaaac agagcaaaca ctgaagaact gcacacaggg gaacatggtc |
| 70381 | taatttgcat ttctaaaaga ccaccctagc ttgaccattt cctgcaggag cgtcactcac |
| 70441 | cacttctgtc ttcctaacag tttttgccag gatcctgcgg gctcctgaat cccacaatgt |
| 70501 | cacctttggc tcctttgtga ccctgcactg tacagcaaca ggcattcctg tccccaccat |
| 70561 | cacctggatt gaaaacggaa atgctgtgag tgtcatgtgt gtggggactt gtctggggaa |
| 70621 | gacccattgg tggtgaactt caggatagac catatagttg tagttaccca gatctctgtc |
| 70681 | attggtctca cctacaccac ttttcaaagc ctcccaatat ctttgtccta gaagccattg |
| 70741 | ccatagaaat caaactccaa gagtttgctt ccttccatgt atcatgagga atactagaaa |
| 70801 | accaatttct ttctttgtgc ctttgtgtaa atgggaagtg gtaataatat cttcccatat |
| 70861 | aagtaaaata attttcatta aactcatcac cgaaagatta gttccctgct agaggctact |
| 70921 | ttgatggaaa gtaatccgga gttagaatca gtgtcagttc agcattcttt tcttcatttt |
| 70981 | caatagtggt aataataact gatgtatatt aagtagttat tttcagccag gctctattcc |
| 71041 | aagcacttta cctacataca tttatttatt tatttattca ttctacaaat atgtattgag |
| 71101 | cttctgaaca tcaaacacta ttctaggccc tgtggataca ggagtaaaca aaacaggcaa |
| 71161 | agttctctgc cctcaaagag cttaccttct catggggaaa tacgggaaaa tatatgttgt |
| 71221 | atgtgtatgg tgtcaaatgc catgcagaaa aataaatcag gaaaaggaga cagaaagttc |
| 71281 | aaccttagga gaaatcagaa ctctcctaca ctgttcatgg gaatgggaat tactacagtc |
| 71341 | actatggaaa acagtatggc ggttactcaa aaagctaaaa atggaattgc catatgatcc |
| 71401 | accaatccac tactgggtat acagtcaaaa gaaagaaaat cagtatatct aagaggtatc |
| 71461 | tgcactccca tgtttattgc ggcactattc acaataacca agatatagaa tcaaactaag |
| 71521 | tatccatcaa tggatgaatg gatacagaaa atgtggtaca tatacacaat gaaagaaata |
| 71581 | ttactaatcc ataagaagaa taaaatcctg ttatttgcaa caacatggat ggaaccgcag |
| 71641 | gacattatgt taagtgaaat aaaccagaca cagcaagaaa agtatttcat gttctctttc |
| 71701 | ataactaaaa aaattgatct catggagata gagagtagaa tgatggatat cagaggctgg |
| 71761 | gaaaggtagt ggggaaggga ggataaagag gggttcgttc atgggtacat aagttagaag |
| 71821 | gaacaatatt gagtgcttag tagctttatt agtcagttct cgcatcgcga taaagaaata |
| 71881 | cctgagactg ggtaatttat aaagaaaaga ggtttaattg gctcatggtt ctgcaggcta |
| 71941 | ttcaggaagc ataatggctt ctgcttctgg ggaggcctcc agaaacttac aatcatggca |
| 72001 | gaaagcaaag tgggagcaag acatctcaca tggccagagc aggaaggaga ggaaaggtgc |
| 72061 | cacacacgtt taaacaacca gatctcacaa ttcactcact cactatcatg agaacagcac |
| 72121 | tgaggggatg gtgttaaccc attcatgaga cttctgtccc catgatccaa tcaccccacg |
| 72181 | ccaggcccaa ctccaacact gggaattaca attcgacttg agatttggtg gggacacaga |
| 72241 | tctaaaccat agcaatagca caacaggaca actatacata gttcacgata ttgaactaca |
| 72301 | tagcacaata caatatttaa gtatttcaaa atatcaagaa gagtggaatt ggaatgttcc |
| 72361 | tagcacaaaa aaatgacaaa ttacttaggt gatggatatc ccaattatcc taatttgatc |
| 72421 | attacacatt gtatgcttat atcaaaattt cacatgtacc ccataaatat gtacaactat |
| 72481 | tatatatcca taaaatttta aattaagaaa cattttaaac agaaagttag acctcaggag |
| 72541 | gaagattata tttttaaaag aatgatcagt gaaggctcat ttggacaaag acatttgaac |
| 72601 | aaagatctga aggagttgaa gggtgaagtt acatagataa ctgggggaac ataactagac |
| 72661 | aaagtctctg agataatggt atacatggca tgtcagaaaa acagcaagga gaccaatgtg |
| 72721 | gttagtgggg agtaagcaaa gggtcaagag atgagaggca ggcattgtgg atcctatagg |
| 72781 | ccgtgtggtc atttagacaa ctttgctttt atttgagtga gatgaggtgc cactgtaggt |
| 72841 | ctgtttctgt tgtctgtttt cccactgatt cttgttcatt ttgtgttgtt tccttgtgtg |
| 72901 | cctagctatc tttgattacc tgctgtgcat tgttttgtat aaaaatatca ggaactattt |
| 72961 | tgaggtctag atgaaggtat tttcctccaa agagaatttt gcttgtttct gccagggaca |
| 73021 | agaggttact gctaatcttg taccaccttc aactaagctc aaggtatgaa gctccttggg |
| 73081 | catcccagat aattataaca tgaccagtaa ttcattatgt ggactattta acttgtagtt |
| 73141 | cacactgata gctccctgat atctagttct tttatatctt aatttattat atctcttatt |
| 73201 | agactcttaa gggcatgaag ttttgattta tctcctgact ccctaaggcc atccaaagga |
| 73261 | aagttcaaat tttctgggtt tggaaaactc tctaagggta aataagattt tcttagtttg |
| 73321 | ttacatctgt taaaaatttt tttttcaaaa ttttaatttt ctcactgtgt agctttgtct |
| 73381 | gaataaccta acatcaacat ctgagacaga aacctaccat ggatttttat tgtcattata |
| 73441 | tagactttaa ccactttggt ttgcaccgag tctttagttt ctgatactct ttatgaggtt |
| 73501 | aagaaaatgt atttttattc ttaatttgat aattattttt aatataaata tattttaaaa |
| 73561 | tcaataaaac acttcatcat caattacatc ctctttttga tgtcattttt cactttaaat |
| 73621 | tgcaaagttt cccttatatt gaaccattct ttcatttctg aatacattgc ttttttgttt |
| 73681 | aattttttaa ttgtgttgtt tcaattttct gtatttcttt caggattatt tgcatctatt |
| 73741 | ttcaataaca gaaatggttt tgtcttttag taggctatca tgatcaggct ttgttaatat |
| 73801 | ttttccaatc ttatagaata gactgccaag tttacatagt ttcctttgat acagagcttg |
| 73861 | aggtgagaaa gttgagatgc aagtctttct tatactccaa gacattttcc tctactatac |
| 73921 | caataattac tgcttctctc ttctccacat cctttctatg tagactccat tgaaatgaat |
| 73981 | ttctttcaaa acttcaatct ttctcttctt tatgctctgc atatgtttgt ggaggaaata |
| 74041 | gggggcccta ttttatctgt caggttattc tcaagctcta gtgtccacca tgattgtcaa |
| 74101 | ctataggaac ccacaacaga aaatctcatt cgcaatctct atctcacagg tagatgcctc |
| 74161 | agcctggtgc catatgcata cattatagct cccagagttc ttcaattact ttgctgactt |
| 74221 | tcttaaagtt ggctcctgct tacagcccct gctatctgaa gtttgaaatt ttccatgact |
| 74281 | ttgtagggaa aatgtatcca ttctcctctc agtaaacctc ttcttttgtg ctaggtttca |
| 74341 | gctacttagt tgatgttaat ttttctatct atcaaatcac ctctttttat cttccagaat |
| 74401 | ttgctggaaa tatctgatcc tttgattata ataccatctt tctatatatt tacttttctt |
| 74461 | aattttgtat ttatctttta ggatttcagg tccataaaca agggttaaac atgtgagcta |
| 74521 | agattattat tactgtaact ctgatgttaa tgaacatgac ttgcatagag cactatctta |
| 74581 | ttattttctt tgtcatttaa aaatattcag cagagcaagt tcttttgcta ctatctaagc |
| 74641 | ctaccttaag caggctctgt gatttttctg gttactatta gatgatttga aatcattaac |
| 74701 | attatcccaa cttagtcatt taagtgtcta gtcctaagta ataaccaaag catccaatgg |
| 74761 | cattcacagt tcctcccttc tcagggatat ttcagcttaa ctttcatata agaaaaaatt |
| 74821 | gcagttggct tcttacctca tgctcactca ttcttccccc atttcctagc tgctctttat |
| 74881 | gaccccagaa ggggtgggtg ggaggtaggg aggagcaata actggtgcaa gagagctcta |
| 74941 | attcaactga tgcacttgta agttggcttt ataccttctg tgcctgccag atatttaaaa |
| 75001 | ctgactctta ccctcataga cctattcatg attcttcaga aatcagggaa cattaagatg |
| 75061 | ataccagtga tgcattttcc tcaggctatc catctttggc cacattagca agtccccaat |
| 75121 | ttgttagttg atggaaagtc tctttgtgtt ggggtctaat cagtccccca aaaagtcatc |
| 75181 | ttgggaggac tgaaaatgac tcccaatcag atcttctata aaaccttttt attgacaaac |
| 75241 | tctggaagcc cagaccattg gactctgtct tctttgttcc ttccattgga gctgccagac |
| 75301 | atcctttaga ttttacaggt gggaattaca tacttgtcta atgtgtctcc aaattgcaag |
| 75361 | tgtgttagag tccccaagat cacattcatg tctgatgatt catgagaagg actcacagat |
| 75421 | tttagataag ctgttatgat tatggcttat tgcaggaaaa ggatatagat taaaatcatc |
| 75481 | aaagaggcat cagttttcct cttctagtga agttgcatgg acagcacttc attctcccag |
| 75541 | caacaatgtg tgacagcatg catgaagtat tgtcaaccaa ggaggctcac cctaaccttg |
| 75601 | gtgtccagag tttttactgg agctcagtta catagatgtg gagtgctcac ataactagtc |
| 75661 | ttagctagtt tccagcccct ccaaaggtca aactaataca acgtggccta ggattccaag |
| 75721 | tattcactat cattcacatt attatcataa accatccagc attgcccaaa atctcagata |
| 75781 | tataaagata ttcttattac acaggatatt ccaaggggca gaggtcatct ctcaggagcc |
| 75841 | aaaggccaga cctttctttg gaatgtgtag ggtttgagca cctcgagccc attgagttag |
| 75901 | cccttcaact ttttactgca cagcagtaaa acacatttat aactctccgg gtggtcccat |
| 75961 | ggaggatact tttatccctt aaggagatgg gtgcatgtcc aacccagtcc tttgggtgga |
| 76021 | ttgagaatgg ctttcaatat ggctgaacaa cccaaacctt tataactcat ctaactggtg |
| 76081 | gctattttca gttttcattt cctccaggac aggctgtcca actttttggt atcttggctg |
| 76141 | tagtgaacat atattatcat attacaatat attaatggta ccatttggag atcaaagaag |
| 76201 | tgtcccaaat cgggacactt taatatccac atcatcccaa attgtgttta atgcaagcat |
| 76261 | cacaacaaga aaatggggga cttcttttta aggaaggcaa aaccttatgg tataattcaa |
| 76321 | atgatattaa gtataatttc tacccaatcc aaggcacatt ccttcatgga cttgaactat |
| 76381 | ttcatgtctt catgactttc tcttctccag gattacatta gtatttaggg ctgccataat |
| 76441 | agaataccac agatagggtg tcttaaaaaa cagaattttt tttcctacag ttctaaaagc |
| 76501 | tggaagtcca aggtcaaggt gctatcaggg ttggtttctg gtaaggcctc tcttcctggc |
| 76561 | ttgcagatgg cttcacatgg ttatctccct gtattctcac agggctttct ctccttgtgt |
| 76621 | gtgcacttct ggtgtctgtg cctacaatga tgagcgccaa aaatcccaga gtgttttttc |
| 76681 | ataacacagt tctcattcca aaatagtttt tattcctctc ttttatagac cattataagg |
| 76741 | tttcttcctc taaattaaat aacagtgact aaaactaagg caaatactct ggagcaaaat |
| 76801 | aaaaactaaa aaacagatag ccttaaagca ggaagttacc ctgtcactag ggcttggcat |
| 76861 | actgtaggct ttagaagcat ttcgcggcat gaaagaatgg taaccctgag gcatctccat |
| 76921 | gtaataagtt aatttgtata ctgcttttaa ataaagttaa acaacttaca gtttcagaaa |
| 76981 | gatagaaggg aagagcattc ggaagactgg gagtgaagtt tcccgggcct gaacccttat |
| 77041 | tcaaaggcgt ctcgacatgg ataacctcga gaacattatg ctaagtgaaa taagccagat |
| 77101 | acagaaaggc aaataccaaa tgatctcact tatatgtgga atctaagagt tgaactcata |
| 77161 | gaagcacaga atagaatagt ggctgtcagg gatgggagga tggggagatg atcatcaaag |
| 77221 | ggtacaaagt ttcagtcatg caagatgaat taagtactgt agatgtaatg tacaatatgg |
| 77281 | tgactacagc taacaacact gtattgcgca cttgaaattt gctaagaggg tagatcctaa |
| 77341 | gtgttctcaa aacacacaca caaaggtaac catgtgagat gatacttcac tagcttgatt |
| 77401 | gcagtaatca tttcataata tacatatata tctcaaaaca tcaccttgtt caccttaaat |
| 77461 | atatacaatt tctatttgtg agttatacca ccaataagct aaaaaaaatt aaaagtctaa |
| 77521 | atttttttaa aggagtttca aaaactaatg ctttgctcta tacttgtacc tggccccacc |
| 77581 | cttgtctctc taaagcaggg tgccctagga agactgattg gctgtagccc caaaaggtga |
| 77641 | gaactaggac ataagataag aaagggaaga agggaaaggg agagaaggaa gtggggaaca |
| 77701 | aagtgcgtca atgtgtgaaa ttttagttgg atgttgtaaa acaggaaaaa aaatgccaaa |
| 77761 | ttctatggct accccaataa caaagaatgt cttatggcaa tggcaccctc caagtcatat |
| 77821 | aattcattta ttattgtcac tttcactatt gttttcgctg aagtcttttt tctgttactt |
| 77881 | gaacaaaatc atttttttcg taatttgtta ataaaattgt ttcattttat ctgtctatcc |
| 77941 | tacatccctt caaatacagt acacaaacaa atgaacgtgc ttttccagct tgtaacttga |
| 78001 | ggcatggtag aatgagaatt agatggggag ttggaagact gaggttgtaa atgcccctct |
| 78061 | ttagttaatg tgattttggg gtccgtttac aactcagaca ctgattttct catatctatc |
| 78121 | tacactaaca cagacgtcag gtggctgtca aagatgatct ctaatggtat aattagagac |
| 78181 | atcagggcta atgttctcta attttgtcca gttatgataa agtaacttgg agtgcctgtt |
| 78241 | ctttacctct ctaagaacgt ttcaagtgtc atttaaaatt tgattcttat ttgtcaatgt |
| 78301 | gttttttggc tcctctgtgg aaactgttat agtcttaagc ctgtcaaggg agtataaaga |
| 78361 | catcctaaga aaatttggag actgggccgg gtgcggtgac tcactcctgt aatcccagca |
| 78421 | cttttggagg ccaaggtggg aggattatga ggtcaggatg gagaccatcc tggctaacac |
| 78481 | ggtgaaaacc tcgtatctac taaaaataca aaaaatttag tcaggcgtgg tggcacatgt |
| 78541 | ctgtagtccc agctacttgg gaggctgagg cagaagaatc acttgaatcc aggaggtgga |
| 78601 | ggttacagtg agccaagatt gcgccactgc actccagtct gggcaacaga gagaaactcc |
| 78661 | atctcaaaaa ataaacaaaa taaaattggg agactgacaa tataacttca ctaatttgtc |
| 78721 | tatgtaaaaa tagtaataaa gaaaatggaa aggtatatac tttttaaatt tctagtattt |
| 78781 | tcaaaagatt catatgaggt tataaatgga agataaatga ttattctctg tcgaagttta |
| 78841 | ggaataacaa gagtaggata gtttaagata gttgaggcct tattgagacc ccaacttatt |
| 78901 | cattcaacat gtgactcaca aaaagtgaaa ggcaaaatag atttatcacc tcagattcta |
| 78961 | aaacaggaat aaaacaaaca aagtgacttt catccaggaa gaaagcaaaa gtacatccgc |
| 79021 | agttcttacg gtcagagagt gtgtgctgct cttagagcca agtttagttc ttggtaattc |
| 79081 | tacccaagtc tgaaaggaaa tcaagatgac tgtccacatt gacgcaaatc acaggacatg |
| 79141 | gaggcaatgt caggcattta atgttgtaaa agttgcaaac cagaaatccc atggaggaga |
| 79201 | acacagagaa tcctatagtc ctgatatcat ttttggtgga atgaacaaag acgaacgcag |
| 79261 | agctgggaga tatttggcac agaatgaaac agagtttgaa ctgtggctct tcactcacta |
| 79321 | cgagttagta aactaatgcc atagcacgct gctcagtaca atgggtactc tgaaagaagt |
| 79381 | gagtgctccc tttctcccct gctgtgaacc tgccagtgaa ccaaaaccct taacagttag |
| 79441 | agccaaagag caggagaaag tccagcaacg tgtcccaaag tgattagaaa ctatcttgct |
| 79501 | ttcctcctat ggaaatacca agcatttttt gtggaaatct gaattcacag atgaagttca |
| 79561 | gagtgaatag taacattcct tttgttttac aatcttggaa atagtagttt ggcatggaaa |
| 79621 | gagcagagct tactttttaa aaagaaaatt atttgtattt ttataaccag atttatttat |
| 79681 | agttatacca gtaatatctc tgttattttg gcatgcaact aaaaaatata gaaaaatttc |
| 79741 | atgtccctct tgctttgtcc aataaatata atttctaaaa tattgtagaa gttgaataat |
| 79801 | tttaacttgt tagaagaatt ttataaaata aacagccaaa tcacctaggt tgaaggttgt |
| 79861 | cttttagaat tttcttaaag ggaggcatat ctatatttag acatattttt aagaggcagt |
| 79921 | agttttgctt taaaatatat gacctgtgaa gtccttttat cttactagct gtgtaaacca |
| 79981 | aaataattac atgtaacaag tcttatgtgg aataattcag cagccagttt gaaagaggct |
| 80041 | aattaatatt tgtggctaca ttaatcagaa aaactatgga aatgtctctt ggtttattat |
| 80101 | ccctttttct atgattcata aacagacatt attacaactg cataaacatg aaccctactg |
| 80161 | ggcaacatcc tttttttttt ttttattttt ttccccaaga tggagtcttg ctctgtcatg |
| 80221 | aggctggagt gcagtggcgc gatctcagct cactgcaacc tccacctcct gggttcaagc |
| 80281 | aatgctcctg cctcagcctc tggagtagct gggactacag gtgggtgcca tcaagcccag |
| 80341 | ctaacttttt gtattcttag tagagacagg gtttcaccat gtgagccagg atggtcttga |
| 80401 | tctcctaacc tcgtgatctg cctgccttgg tcttccaaag cgctgggatt acaggcatga |
| 80461 | gccactgcac ccggccctgg gccatattct aacagttcca caggatattt ccagccagct |
| 80521 | ggaactatct gcaagacagg gccattgagg ccagaatacc tttcagaggt cttccctagc |
| 80581 | taaggtcaaa tcataaaatt atcggattat ccatagctgg gggtggattc tctcaagacc |
| 80641 | gcattagtaa tgtattaatg atacattaag aagtactcca caggtctata actgctcaat |
| 80701 | caccttcaaa gagtttttac acacattatg tcaggcatga attattatgc ctatttaata |
| 80761 | gagcttccta tcctactgcc tctttttaga aaaaaaagtt ttattgtgat ctttattgta |
| 80821 | tacatttaag gtacaaaaca tgatgtttag ataaacatat acagtgaaat gattactata |
| 80881 | gtcaagcaaa ttaacaagtc catctcctca catagttaca gctaacttag aaagacagaa |
| 80941 | catcacaccc agagcaggga gctgtgtctc cagctccctc aggtccatat acctgagtga |
| 81001 | cttggagaaa atcatgcatt tcctgtaact accagacgta gtcacctcaa ttaagtaatc |
| 81061 | tgttttgctt aggcatacct tggtaaaagc tcacagtact aatctggacc cacccctttg |
| 81121 | atctttgaaa cctgaggtgc gaaaaaatat caaaatggag ggcagaggga tttgttttta |
| 81181 | ttttcagatc atcaaaaata ctaaactgtt gatcagaacc atccatgaag aaagaaaaac |
| 81241 | aaattcaaac tatgactaat ggggcctgct gtgcgtgagt cacaaactga atcaagcaag |
| 81301 | gcctctggaa gcagggggaa aagcattcag gagtcacttt gtactcaagc atgaacctcc |
| 81361 | taaagtgttg gtctctttgc tgaactattt caacacctgg atgaagtcca ggagaagaga |
| 81421 | cgtcaggcag tttaaaaatt gcttctcttt cctactaagg atgttttcag tgaccttgag |
| 81481 | gaggtgttta agaatgactt tcttaggtgt gaacatgaaa acagtgggac ttagggcaac |
| 81541 | atttggggag attccttgct tcaaagttgt ctctaaggca ttgcacattc ctacttcttg |
| 81601 | caggagggaa ctgtctttcc aaaggatttt gcaaactgtt ctgcttttgc ttatctaaac |
| 81661 | tggaaagaga tatataaaca aaatataaaa tacaaaataa cataaagtct gttgattgag |
| 81721 | catagaggct tggggtcttt agcttttttt cttattgttg ctttgtatta aaattcaagg |
| 81781 | gagtccaaat attttcagtc cccttgatct aactgggata ctcatacaat gagacattgc |
| 81841 | acttctgata aatatgataa tgatgagttt agtttgaatt cccaactgag tgcatgtctc |
| 81901 | ttaaaaacat ttttctcttc actttgtaaa tttcttatag agtctaggtt gctcatctgg |
| 81961 | ggaaaatgcc acaatacttg atctgacctg gaaagattcc cagtgaaggt aaattgatac |
| 82021 | tcacataagc cagtttttat cataagaatt gagactagta ctgaaggtaa agaataaaaa |
| 82081 | gctagttgga caaacgttca ctgcatgatt gctacacagt gcttggtgcc agggatagcc |
| 82141 | agccacctgg cagctaggaa gaaagctggc cttacctcag agctttcttc ttcctggcta |
| 82201 | gtcaaactgt gaaaatcctt tctcatgcaa cacaataaaa taattggctc caaaatgtct |
| 82261 | cattctcagc ctcattccct aatactggac acaggaatct ctttcagcca agcagccaag |
| 82321 | ctcaggccaa catgggacgt acctccactc ctttaagata aagtcaaagc ataacaccag |
| 82381 | ctgattcctt aactgtcatt tcactccagt ttccctcaat tggcatcatg tttccctgat |
| 82441 | ataagcagcg ttaattaaag attagttatt gaaaaggtgg tattgtgctc aagaaaacag |
| 82501 | tgaaaaaaga accaccattt aaaaaaaatg ccaccacctt aacaatgaca tttgtcagat |
| 82561 | atgtattttg ttcatggttg gttgatttgt taaggtagtt gttactcttg ttttctaatg |
| 82621 | actgctcatt tgttctctct tctaggacta gggaaggcac tgtaacataa tgattaaaag |
| 82681 | cataggcttt ggtgtcagac acgctaggat gcaaattcta actatgcaac cttgtaaaat |
| 82741 | gtgctgggca gctatctttc ctacagagtt ggagtaatgt tgactaatat atatgcattt |
| 82801 | gtataaatta agctgttaat tgtgtgtcaa tattgcattg atatcttggc tctttaagga |
| 82861 | catttgattt tctggtttaa gtagtacctc ctgcatgaac ttcctgacgt gttgaactat |
| 82921 | ttcaacacct ggatgaagtc caggagaaga gaggtcaggc agtttaaaaa ctgcttctgt |
| 82981 | ttcctactaa ggatgttttc ggtgaccttg aggtattatg tatgtattat gtatgtcatg |
| 83041 | cctcagagac agacttggct tcacaaccat ttttcctgac aaccatccac ctatctgaca |
| 83101 | tgcatttatg gggaactgaa caggagttat gaaaataagt ccttaagtct gcaacctctc |
| 83161 | atacttaact gtcatctgta ggacactgaa gacatatatc agaggatcca caccgtatat |
| 83221 | aattcatgcc ttgtattggt tatctatggt tgtatcacaa atcagcccaa agcttagtga |
| 83281 | cttaaaccaa taactattag tgctcacaag tctgtggttc aactgggtgg ttttgctgat |
| 83341 | ttggttttgc tgctgggcag gactgcctga tcttggctgg tcaagttcct gtgatcggca |
| 83401 | gattgaaaag gtggtagttg gttcagaata gccttatgcg taccttggat ggttgttgtt |
| 83461 | ggttcccacc tggggcagag gggataaacg ggccaggtgt gtttcatctt ctaccatgct |
| 83521 | ggctcttgct tgtttatagg cagtcccagg ttccataaac aagaaagcaa gccacaatgt |
| 83581 | gcaagaacgt tagtttctgc tcattcattc caaaacacat cacaaggcta atctagattc |
| 83641 | aaattatgga ggaaaatgct acttcttgag ggaagagcta caaagtcaca ttgcaagggc |
| 83701 | atggacacag ggagggaaaa tcttttggct atgtttgcag tgtaccacat gcacttgatt |
| 83761 | ataatcttaa tgcttttccc ttcaaatcta ctgacatagt atagtgggaa atccttgact |
| 83821 | gagttctttt attttccttt actctgtcag gtttcttctg ggtccattca agagagtgtg |
| 83881 | aaagaccgag tgattgactc aagactgcag ctgtttatca ccaagccagg actctacaca |
| 83941 | tgcatagcta ccaataagca tggggagaag ttcagtactg ccaaggctgc agccaccatc |
| 84001 | agcatagcag gtaggatgcc ccttcacatt tgcgttgttc caggagaggg gagagttgat |
| 84061 | actattctac aatatcgagc attggagaaa atctcccttt tcttttatct cttatttcag |
| 84121 | ttgcatttgg gctatttcct cccccatggg gatactccgg aggtgaccta gatatggatt |
| 84181 | ttggcagatt tgctgcctgg aaaactaaat cctcaatacc tacccaagtg gtaggtaaca |
| 84241 | gcttccagga actcacttct tccttccttc ttgtttttct tatctttttt ccttttctcc |
| 84301 | atttctcttt cctccctccc tccttccctc cctcgctccc ttttttcctt ccttctttcc |
| 84361 | ttccttccct tattccctcc ttcattcctc cctccttccc tccctccctc ttcccttccc |
| 84421 | tccctccctc ttttcctttt ctttaatttc ctttcctttt ccccactcct cgttctctct |
| 84481 | tttggttgat caaactttcc aagtctctca tggagaaagt tggcagacat tcaacatggt |
| 84541 | catgccaatt tctgcatggc tatttcttgc gggcattttt acctgtgagc atgatccctg |
| 84601 | tggtctttcc cttccctgct tcagcaagcc acagcaggtc ttacaggtgt tttcaagaaa |
| 84661 | aaggctgaca cacggatttc taggatgttt gtgttgagtt ctaaaggttt atttttaaaa |
| 84721 | tgtaaatgtc ccaaaggctt ctgagaacat ttatagaatt cctagagatg gaaataatcc |
| 84781 | ctgtatcagg aaatgccagg catattagat tcatagcgtg cctagggctc aagggtcagg |
| 84841 | agagaggtgc ttggcctgac atgtaaggca agcacggata gagcttgtgc ctagggccaa |
| 84901 | ctccccacag cctgctgttt cttattacag tgccacctct ttgatcttcc ctgatgggga |
| 84961 | actttccagt aatgtctgtg atttgtaata tgaggtgaat aaaaaccaca gataaatgat |
| 85021 | aaggagcact ggacaaggct ggccctctgg gactgcctat gaaggaaaag gaagacttca |
| 85081 | aaattaaggt tccctctcct ttctcccccc agtgatatct ctgcctacca tgtcttcaga |
| 85141 | caccatatca gttattctag catttccaca ggtgggaaat tatttctccc agaaaatgaa |
| 85201 | aagtcttcta aaagtaaatt caactcaatt taacaaataa taataatatc acataataag |
| 85261 | taatagtata atcatataat ataaatagta ataagaatta ctatttacat agcactttgc |
| 85321 | tgtttataaa gcacttttac atgcatttat caaatactct agatgacgcc cattgtgttg |
| 85381 | ggtagagtaa gagatgaaaa gttaatttaa aaatgggtcc tgccttccag ggactctgca |
| 85441 | tctagaagga agtcagtcag tcacgtatat tatggcactg gctgtattct attgcagaag |
| 85501 | catacacaaa atacaacagc agaacaagag aggcaggatg tcagggaatc cttggaaaag |
| 85561 | aggtatcatt ggagcagaag atgtggatat gagatgacta ggttgaagtc aaacctttca |
| 85621 | aacctgttca aaaaacagca aatcagtggt caaggctaga ccacgagaga gacagcaggg |
| 85681 | aggtggaaag tggaaacaaa agttgaaaca tgggctctag ccaggttatg aagagcctgc |
| 85741 | ataggtttag tatattattt cattgatgag aagtcattta atgatttaga agaaataagt |
| 85801 | gcaatagata tatcctctag caaattaaca gatgctgagg tgttaggagg ttgagaagtg |
| 85861 | cggcaagctg tcaggaggct cttccaggtg tctcaaaaag tcatgaaggt ctgaacagca |
| 85921 | gcagcagcat tggttcaaag ggaagaggag agatgcacag gacaaatcag gatagaaata |
| 85981 | aggtttgcaa atgcttgaaa atggcagtgg gctagaagga tgagttcaga tgtgtctgat |
| 86041 | tttgaaaggt gcccaggaca agcaataata tataaacaca aagcatatga acaggagaag |
| 86101 | gtccaggaga taatagaata ctcaaaaatg ttgatgcctt tgaattacct ggaattattc |
| 86161 | tggtctcact gtctttgtat tatttgacat ttgatcaaga cctactgtta aataaacata |
| 86221 | tatttataaa aatctatcaa tatatattta tataacatat atgtatgtat gtgtgtttat |
| 86281 | atacacacac atggagagag agagagaatc acatttaaat atgaatactc catccaagaa |
| 86341 | aataaactga tctactttaa tttaacctgc actttcactg gcagaagaaa aatactaatg |
| 86401 | ttactaatct taggagagtc tcatttcagg tcattattca gtcactcaaa gatacccgtg |
| 86461 | ccagagctct ttctcccttt tcttccttct ttctctttcc ctagatggca aaggagaaga |
| 86521 | ggatttgtat ttaggcagtg gtgtcttctg gacacccccc acaccactgc taacccccgg |
| 86581 | atctctagtg aagtctgcag ctgaacttgt gattttatct cttggcttgg ttagggccca |
| 86641 | ggggacctcc ctggctggct tagcctgttt gagacatctt ttggagcagc acagccctct |
| 86701 | gcatctcagc caaatccttc atcatgccat tgtctacact ctgctctgca acctcagaag |
| 86761 | ctaggtctcc ctgtctgtcc ctactgtggc cctgtggctg gcctgctgaa ttccaactca |
| 86821 | gctccttcag tactcctcac agggcctgaa gcaacttcta gatcactttc aaggcataca |
| 86881 | gaattcagga gagatttggg cttgctaact cagttctgct gtctgcccaa ccttgtcatg |
| 86941 | ctaccatttt cacattaatg ttgccatcca tgttggagta agggatccct gtggggtgtt |
| 87001 | ttacttatag catcctaaat aagaagttag aaaaactcca tctgcttttg gctttttcct |
| 87061 | taattgatca atacacctct attctaggat ttgcattcag aaaagggatc tataaaatct |
| 87121 | ataaaatatc caaattttta atctccctac cttcctcctt ttcacagcat atgaagctgg |
| 87181 | aagagcaggt ggtcaggaat ctcccccaac cttcagtgca gtaatcttta gagcctactc |
| 87241 | atcataagta caaaggtggt tgtgaaggca gtgctataac cactgagggg aacctaataa |
| 87301 | aatgggtata tctactccac acaagggaag attcatccca tggacacaat ctttaagatg |
| 87361 | ccgagaggac tacagggcat gattgccacc atactatatg cagcaacatc atgacttcat |
| 87421 | tcctgtccta gcccagatga gaacctgatc caactccata aacggaacac caacagggaa |
| 87481 | atctgtaacc ttaaaaaaac ggcatcttcc agtggtagaa agagcctaaa ggtctgagac |
| 87541 | aggcagtata ggaaggccat gtcctagggt gtagggagag gtcatcttag atatcagtgt |
| 87601 | tgagaaagac cagaatgagg atggggacaa tcagcaatgg ccacccagga aaaataacag |
| 87661 | tggaatttgc atgggggaag ggatggtggg agcaatgcaa agattggcat gtggcagcag |
| 87721 | agagcaactt gcaatctgct gcagcagcag aggtgagtgc ctatgaagag cagttatctc |
| 87781 | tgtaggaata atggttgtgt agactagaac cagctatgac ttttcatgag cttgacaatg |
| 87841 | gcagtgcgat ggaggtcagc tttagagtgg ggagaagttg gggaccctgg aagtgaggct |
| 87901 | tgaagccact ctggacaggg gtatgtttac agtagctacc caaggaactt gggtagaggt |
| 87961 | agagaaggca gcataaggag agggttaacc tataactggg acctgggact ttgctcttct |
| 88021 | gaaccacaaa ggcaacaatt aaatctgacc atatacagaa tcatagtgct gcacgatgtt |
| 88081 | cggagtcaag taaagaaatg agagttgtta ctcgaaaact gaaaaatgga aaagactaaa |
| 88141 | aaggtctaga gtggatatga taaatgagaa acatgcctat taattaaaaa attgctacac |
| 88201 | atctagtgtt attccctgga ataagaccct tttgtccaac ccctgatttg tttgtctggt |
| 88261 | tgattctcac tgattattag gggacttgac actcctcgac ttttcccaaa atttcccgag |
| 88321 | gcagacctaa taagggtgtg tcatgagtgt gattgaataa gacttctgaa ctcccatgag |
| 88381 | acaacttcca ttttctcttc ctctttgacc tgctcttgtg agttagacct gctagaagcc |
| 88441 | ccatgaaggt ccctctggaa tcactgagga aactacctcc caacactggg gtgctccgtg |
| 88501 | gactgatatt ttagggaaat gaggctactt cgatgaaaaa gggatccttg gagcctcttt |
| 88561 | cattcagggt catttgaaag aagcatggcc ttatgctccc cagatctccc agaactcaag |
| 88621 | tatggtatct gctttctcca tttctgcttt tggcccctct ctcggcatcc gatgaggctg |
| 88681 | gccagaagtc cagaacttca agtcagacat ggcgcctctt ttgccattct ggcaatgctt |
| 88741 | ccttaatgcc aaactctgct gtctcattta accatctcca tcctccccta tgtgactagt |
| 88801 | acccctagta gtcaacagcc ccctctgtca tgttcctcgt gtttcccttt gaaatttcta |
| 88861 | ctccatctgt ttatttgctc ttcccagcag gaaaactgga aatggaaaaa gctgatctta |
| 88921 | tgcttatgct taggattatg tctaatgata tttctagtgt ttgaaggaca acaaaaaata |
| 88981 | aagatcaggc ttcatttttc caaaaccctt ccatgtgtct gcccaaatcc ttatgcattg |
| 89041 | ttttagaaat gtgatatatt tctttctgac tcatttacat ttactgttag tcatcaaaag |
| 89101 | gagtcagaaa aacttttgga acctctccct atgaaccagg aagttgcatt tcccctccag |
| 89161 | tcacaacccc ataaagcctc aagttgcaaa gtaaacttgc caggatgctg ggcatttggc |
| 89221 | aagatgcttc tctttgtcat cagaaccctc agccctgtgg aagaaagcaa gacagcctat |
| 89281 | gataccaata gtgagcatag tcctaaaaca gccggagacc atgaaaatgg acacagggat |
| 89341 | tcagaagaga aaaatacagg ccgggtgtgg tgactcacac ctgtaacccc aacactttgg |
| 89401 | gagtccgagg tgggcagatc acctgaagtc aggagttcga gaccagcttg ggcaacatgg |
| 89461 | tgaaactcca tctctactaa aaaatacaaa aattagccag gtatggtgac gcatgcctgt |
| 89521 | aatcccagct acttgggagg ccgaggcatg agagtcactt gcacctggga gatggaggct |
| 89581 | gcaatgagct gaaattgcac cactgcacca cggcactcca gcctgagcaa tagagtgaga |
| 89641 | cttcatctca aaaaggaaaa aaaaaaaaaa gagagagaga gagagagaga aataaaatac |
| 89701 | atatttgtat ttcccattct caatggcacc aacaggtcct tcatcagaga gtgcagtcca |
| 89761 | tggttctatc catgtttatc cagtctcaga gtacagggac gagcctatgg tcacacaccc |
| 89821 | ctctaatgta agccgctccc cattctacca acaaccccca ctttctatat tgccccttct |
| 89881 | ttgaatcgac ctcagggtct aagccttttg gtcactaaat ttaaaagtca catgaataca |
| 89941 | agtagtatca aaactgagca ggagggaaaa atctgatgtg gtaatactgg agtctcccaa |
| 90001 | actccccaga tgaaccacat ggttttattt ttcttatttc aaaagaggtg atgtttcaca |
| 90061 | ctactacaga aaaaaaaaat ggaactttga acactatttc ataatcagta tagctgattg |
| 90121 | aatgtactga catgtttcat gctctttctc atgtctgata gccacactcc tgtttcctcc |
| 90181 | caatgttgga gataattctt gagacttggt aacagagcag tagcacgtaa tttgctttac |
| 90241 | cactcagtgc ttcaaccatc ataggtaaag agtgttctta tcaacaaaac agacaccatc |
| 90301 | cctggcattc catgctggct ttctctggtt ttaaattaat taaagtcatt ttgtttttgt |
| 90361 | tcttaataaa agggaaaagg ataaatgagt aacagccagt agccacatta aattgctacc |
| 90421 | agatgttctg cagttaggca taaaaggtaa gcatcctttt acaatcaggc agctttctta |
| 90481 | tattagaaca gttctgtttc agaagagagc caatgttttt gctgacttct agataatttt |
| 90541 | aaagatacta taatattggt ggatatttta agaattttta aatactacaa atattggtgg |
| 90601 | gatattttga gaatttttta aaattgaatt ctgagcttaa agcactttat agttgcaagg |
| 90661 | ttcttggacc ccttcctcat ctcctagcca tacaccccca caaaaataaa agaaaatgca |
| 90721 | agtggagaca tttcctatag aagctcatta tcccctccca ggtactatgg ctggattctc |
| 90781 | ttttgcattg gcttatctcc atgctccttc cacattacaa aatgagggct ctatcattta |
| 90841 | cataaccttg gcccccttgc ttttcaatag atcttcctgc tcagctgcta ttagaatctg |
| 90901 | cctttgtatt tccaaattat atgccatttt ctctggaaga attttccagt gttccactgt |
| 90961 | ctttcttccc actattgtat ttgtataatg gcagaactgc catgagattt gggggcatga |
| 91021 | aggcacttct ttcctgagtt cagcagtgac atagagttct cttcattctc tttcatagcc |
| 91081 | tttggtcatt ttaattaaga gactcttgca ggcctctagc ccggttcact aaaatttaaa |
| 91141 | ttctctctaa tttataattt gttgacatca ttggcatcac ctcttagatg atcaaaatga |
| 91201 | caatcctata acccagcctg cctatctcag taaaacagaa agaatctact agatgttttt |
| 91261 | cagaatgtaa atagatataa catttataat gattttccta ttgtccagca aattatctcg |
| 91321 | cagttggagt cctgcaaatg ttagagacat cttgatgttt ccttcatagt cactccagag |
| 91381 | tctgttagtt cttatgtata aagaaatttc acaaagaaat ccagcttttc tcactgctaa |
| 91441 | aaggatgtgg ccttctgtcc ctaagcaaag tgacccattt aggggaggga taaaatttta |
| 91501 | gagcttatta ttggctaaaa ccctactctg gatcaaaagt ttgtttttaa agtgcattgt |
| 91561 | agattgcaga acaactatga atcactcata aagagaggta acaaagacct gaggctatat |
| 91621 | gtgtaaccag tgtattgaac aatttaaacc tgacaggtac acagagaaaa tggtgttttc |
| 91681 | tccaaccaat ggggactgag gaattatgcc ttttagaatc tccttgccct tctcaagagc |
| 91741 | aaacccttta caaactttca ggcagattcc ataatttcca ggtaatgttt agagattaaa |
| 91801 | aacccagcct gatctagttt aaaactctcc tcttacataa tttaaacctc atctgcttca |
| 91861 | ctctctccac tgccagccaa tcttccattc aggaacatgg agagtagaca aaatgggtct |
| 91921 | gctactatta tacacccttc ccttcttccc tttcctcctg ttcctcttca tctctggcat |
| 91981 | atttaaggag aagaggattt acaaggaagg cggcaaatgt gttttcctaa atggaatatg |
| 92041 | ttttagttct ttctctgtta attgttattg cttctctgac taataataat aatgttaaca |
| 92101 | attgcaaggt acattctcag tgcccagtgc catatatata tacatatata tatatatata |
| 92161 | cacatatata tatacatata tatatataca tatatatata tatatatgaa tttatttaac |
| 92221 | acccacaact caatgaaatg gatactttta ttatccccat ttcacagatg agcaaactaa |
| 92281 | tgctcagaaa ggtgcccagt ctgacacagc tagtaaatgg cagaaccggt attgaaaccc |
| 92341 | agcaggtgca gcatccaggc ttttaatgcc ttgatgggtt attaatgtct ctctattgca |
| 92401 | ggtggtacct gaatactgac tgtctcctag gacatttggg ttctttggag ggtccttcgg |
| 92461 | aagactgtgt attgcacagt gccctgatgt gggagattct ttactggttc aacctctcat |
| 92521 | aatccccagt ccttgatctc aatccctatt gaggtaacct gcaattgctt atgactgggg |
| 92581 | ttctttgtct ggtgtccagt cttcttggta acttagctac cttcatagca tccactgaac |
| 92641 | acacaggaat gtcccatgtc cctaccatcc caatggtggg gagttctatt cctggaccct |
| 92701 | ctcactctgc cctcagattg ttgcaattct ttttagacag taatattatt aaaaaaaaaa |
| 92761 | aaagacaaaa caaatgcttt ccttgtctct gagtcctagt ctcttaattc ttaggcagtt |
| 92821 | gaaaaatgac tccatgagat gtgttgttac catttacatt gtatcattca agaatgagaa |
| 92881 | gatctaggga aataattcat cccctttctc cttatatttg ctgagatgcc cttcatcttt |
| 92941 | acttgaaggc tgagaaccaa agctatttgt ctggttatag ggcctacaaa ttgcaacaaa |
| 93001 | ggttggggtg attgtgcctt ttagtaaatt ccaaggaaaa atgtcaagtt gtatgtgtgt |
| 93061 | gtgtgtctgt gtatgtttac atatttccta cgtgtgtgtg tctgtgtgtg tgtgtttaca |
| 93121 | tgtttcctat aatcatttgt cttgcttttt gagggcattc caactactgg tggaaaaaat |
| 93181 | aaataaatac aagggggtcc atgctgtgag tagttgaaat gaaaatactg gtgttttgat |
| 93241 | gggattaatt cagtacattt ataatagact aattcaccca aaatcaactt ctaaaaccag |
| 93301 | tttgccttaa ctcacttaac tcaatttcag ttagcctgat gactattgca tctaatgttg |
| 93361 | aaaattcaga attaaaaatg aaatattggc tggatacagt ggctcatgcc tgtatcccag |
| 93421 | cactttggga ggctgaggcg ggtggataat ttgaggttgg gagttcgaga ctagcctggc |
| 93481 | caacatggtg ataccccatc tctactaaaa atacaaaaat tagccaggca gggcagcagg |
| 93541 | tgcctgtagt cccagctact caggaggctg aagcaggata atcgcttgaa cctgggaggc |
| 93601 | aaaggttaca gtgagccaag attgtgccac tgagctccag cctgggcaac agagtgagac |
| 93661 | tctgtcttga aaaaaaaaaa aaagaaaaag aaagaaagaa acatttacca aataatatgg |
| 93721 | ctgagtattt atgactaatt tttgtattgc taatacttta tcacatatag ggaatcatga |
| 93781 | gattgttaca tattgttgtg actgtctttt tctgttccag tcattttgaa cacttgcatt |
| 93841 | attctttagc caatttgtca aaaaaaaaaa gtctttttct ttacaatttt gaatttctgc |
| 93901 | taaaaatatt tagtggaatg aaatttattc cattaaaaaa acgaaccttt tgattattat |
| 93961 | tgtctccttg ctactttcct ttattataag agtctatgaa ctctacatgt atgtgtatat |
| 94021 | atatacatat gtatgcatta accaaattag catactctca gagctcattt ctgtttagtg |
| 94081 | aggtattcgt attttaaaag aataaagaaa aatgggtccc tcttttgatc accattccat |
| 94141 | gttatttatc aagagatgta attgataaag gttttatttt gttttgtttt gagacagagt |
| 94201 | ctctctctct tcctcccaag ttggaatgca gtggtgtgtg atctcggctc attgcagcct |
| 94261 | ctgcctgagc tcaagagatc ctcctgccac agccttccaa atagctggga ctacaggtgt |
| 94321 | gtgccataac gcccagctaa tttttgtatt ttttgtaaag aaggagtttc accatgttgc |
| 94381 | tcagactggt cttgaattcc tgggctcaaa tgatcctcct gcctgggtct cccaaagtgc |
| 94441 | tggaattaca ggcatgagcc accatgcctg gccctaattg ataaagtttt aaaatgcaaa |
| 94501 | atgtgcatca tttccaaatg aatttatagt gagaatcata acattttgaa aaacagtttt |
| 94561 | caaaatgcca ctggagtgtt aacaaattaa taccatctgt gatgactttt ccattctgcc |
| 94621 | aaaacctgat aaagctctgc tgaagtaaag aaattggttt cacagagcca gcacactgaa |
| 94681 | tgggcaaaag ctggaagtat tccccttgaa aaccagaata agacaaggat gcccactctc |
| 94741 | accactccta ttcaacatag tactagaagc cctggccata gcaatcagat aagggaaaga |
| 94801 | agtaaaaggc atccaaatag gaagagggga actcaaagta tccctgtttg cagataacat |
| 94861 | gattctatat ctagaaaacc ccatagtctc tgcccaaaag ctccttgagc tgttaagcaa |
| 94921 | catcagcaaa gtttcagggt acaaaattaa tgtacaaaag tcagtaaatt cctatatacc |
| 94981 | aacaatcacc aagccaaatc agaaacacaa tcccattcac gattgccaca aaagaataaa |
| 95041 | taccgaggaa tacagctaac caggaaggtg aaagatctct gcaataagaa ttacaaaaca |
| 95101 | ctgctcaatg tgcttaaagc aatttataga ttcaatgcta ctgctatcaa acaaccaacg |
| 95161 | acattcttca cagaactaga aacaaaacta ttctaaaatt catatagaat caaaaaaggg |
| 95221 | cccaaacagc caaggcaacc ctaaacaaaa agaacaaagc tggaggaatc atgctatctt |
| 95281 | acttcaaact atactacagg actgcagtaa ccaaaacagc atggtactgg tacaaaaaca |
| 95341 | gacatataga ccactggaag agaatagaga gctcagaaat aaaaccacac acttacaacc |
| 95401 | atctagtctt aggcaaagct ggcaaaaaca ggcaatgggg aaagaatccc tatttaataa |
| 95461 | atggtgctgt gatagcaggc tagccatatg taaaagattg aagctggatc ccttccttac |
| 95521 | accatataca aaaatcaact caagatggat tatagaccta ataaaaccta aaacagtaaa |
| 95581 | cctgaaagat aacttaggaa atacattttg gccataggaa ctggcaaaaa tttcatgatg |
| 95641 | aaaatgccaa aagcacttgc agccaaagca aatctgacaa atggaactta attaaactta |
| 95701 | agagcttcta catagcaaaa gaaactatca acagagtaaa caggcaactt acagagtgga |
| 95761 | gaaaatattt acaaaccata catctgacaa aggtctaata tttcagaatc tacagggaaa |
| 95821 | ttatacatac aaggaaaaaa acaaacaacc tcatttaaaa gtgagcaaag gacataaaca |
| 95881 | gacacttttg aaaagaagac atacatgcag ccaacaagca tatgaaaaaa tgctcaatat |
| 95941 | cactaatcat tagagaaatg caaatcaaaa ccacaatgag ataccatgtc actccagtta |
| 96001 | gagtggctat tattaaaaag tcaaaacata acagatgctg tctgagacca gcctggacaa |
| 96061 | catggcaaaa cctcgtctct actagaaatc caaaagttag ccaggtgtgg tggcacatgc |
| 96121 | ctgtaatccc agctgcttgg gaggctgagg caggaaaata acttgaaccc aggaggcaga |
| 96181 | ggctgcagtg agccaagatc acaccactgc actccctcct gggccacaca gtgagactcc |
| 96241 | acctcaaaag aaataaaaaa ttaaaaaata acagacactg gcgaggttgc tgagaaaagg |
| 96301 | gaactcttat acactgctgc tggtggtgta aattagttca accactgtgg aaagcagtgt |
| 96361 | ggtaagtcct aagataacaa aaacagaact accattcgac ccagcaattc cgctactgag |
| 96421 | tatatattca aaggaatata aatcagtcta ccataaagac acatgcatgc aaatgttcgt |
| 96481 | tgcagcacta ttcataatag ccaagatgtg gaatcaacct aaatgcctat caatgacaga |
| 96541 | ttggacaaag gaaatgtggc acatatacac catggaatac tacgtggtca taaaaaataa |
| 96601 | taagatcatg tcctttgcag gaacatgtat ggaactggag gccactatcc ttagcaaact |
| 96661 | aatgcaggaa caggaaacta aatactgcat gttcccactt ataagtggga gctaaataat |
| 96721 | gagaacacat ggacagaaag aggggaacaa cagacactgg gatctacctg agggtagagg |
| 96781 | gttggagaaa ggagaggatt aggaaaaaaa aaaatagaac aactttcggg cactatggtt |
| 96841 | agtacgcggg tgatgaaata atctatacac caaactcgca agtcacaagt ttacctacac |
| 96901 | aacaaacctg aattatgtac ccccgaacct aaaatcaaag ttaaaatatt cttcaggatc |
| 96961 | actgaataaa aagctcataa agattcaaaa aagtcataca ctcaaagtta taaaaacctt |
| 97021 | ctagttacca accctcacca atctcaaaga gaatgattag aaatacaagc tcattgtatt |
| 97081 | ttaagaagat tgacctctca gagaaattca tgttacagcc cttaaaaatc ccccccaaaa |
| 97141 | taacacccaa atggatctaa aaccttcaaa acggagttag acctcaacat taggagaaat |
| 97201 | tttaatatgg caaattggtt ttagacaaag catatgtttt actgtttatt ttcgtaatga |
| 97261 | gggttgagag gacaggacga agtaaccata aagcacttgg taaagagtgt gaagttttta |
| 97321 | atttacaaga aagaaaatgg aataattcag aaatgctggt tatatcacac ccttgccggg |
| 97381 | attctcatgc cttggcagag tctgagttgg ggctgtttgt cctgggaatc acataccacc |
| 97441 | agaggagagg cgtattctcc tcagagccct gagtttttac atccgtgagc atgggcagct |
| 97501 | acataatttg cagggctagg tgttccaaaa acggtaagga tttcaaggta gggagagcag |
| 97561 | aacattaaat taagcccgag gcccttctga gtgcagatgc catgcagcta cccagggcac |
| 97621 | acagccttga ggcagccagg tctatgagaa caaccattgc ctactttctc ttctaccctc |
| 97681 | tctcctctgc atgttctttt cttttctcct tctccttctt ttttcttttt ttccaagtct |
| 97741 | gctttatgca tcttctctgt taacctccac atcttgcttt tttttttttt tttttttttt |
| 97801 | tttgagacgg agtctcgctg tcgcccaggc tggagtgcac tggcgcgatc ttggttcact |
| 97861 | gcaggctccg ccccccgggg gttcacgcca ttctcctgcc tcagcctccc acgtagcggg |
| 97921 | attacaggcg cccgccacct cgcccggcta attttttgta tttttagtag agatggggtt |
| 97981 | tcaccgtgtt agccaggatg gtctcgatct cctgacctca tgatccgccc acctcggcct |
| 98041 | tccaaagtgc tgggattaca ggcgtgagcc accgcgcccg gcccacatct tgatttctta |
| 98101 | atgtaaagtc cctgggtagc taatggaggg aaattctgag cttagatatt gccttggtgg |
| 98161 | atgttaccca tgattgactc agtttgaaaa gaacctgctt cctaaagata ctcaatattt |
| 98221 | tcttcatttc ctccgaatct gcccattaat gcagcatctg gcctcctagc agaagcggag |
| 98281 | aacttttcag aaacttagag atcagtagta tcaaaacaat ctcagccaaa gcataagatg |
| 98341 | aaaatgtact aacgttcttt cttttctcct ttaatttgac ttcctgtggg aacttccttt |
| 98401 | cctgttaata gaatggaggt aagaaactgt tattgtaaca attgtttcca atgttttgtt |
| 98461 | ttgtagggat ttcgtttgtt tgtttttgtc tgtgagaggg tcttgtgttg cccaggctgg |
| 98521 | agtgcggtgg agtatgtttt gtttttgtta tttgacatgc catttattga gtgtttacta |
| 98581 | tttgctgagc actgtaacat tttagaaact ttctaatttc taatcctttt agtaagtatg |
| 98641 | atgaagtgaa agacaggtaa cctctactga atacttacta tggatcgggc actgtgctga |
| 98701 | gcttcacatg cattatttaa atctatcttc aagacatccc catggaatgg gtattattgt |
| 98761 | tatctctatt ttgcagaaaa aaaaaataaa aaaacctaag aaagtttctt tgaactggag |
| 98821 | agttcaagtg gcttacacag ccagtaaatg ataaagttag aaatggaacc cagaactgcc |
| 98881 | aaactccccc ttatagctct aacctatata taagaaggta tcccttgcaa tataaccttt |
| 98941 | tgcaacttac agtgttttat ttgcagtact tttaaaactg accttcataa aactctatgg |
| 99001 | agtgtaatta attttaagac agcattctta gcacatagcc tacgtttaaa aacaggcagc |
| 99061 | aggatacaaa attttggtta gatagcaaaa ataagttcaa gagatttaga gtacaataca |
| 99121 | gtgactatca ttaataacaa tgtattgtat tcttgacaac cactgagaat agattttaag |
| 99181 | tgttctcatc atcagaaaat ggtaagtatg tgaggtaatg catttgttaa ttagctcaat |
| 99241 | ttagtcatcc cacaatgcgt acatgtttta aaacatcatg tggtacacaa taaaaaaaac |
| 99301 | agtttttttt aactaaaaac accttttaaa gttttactgt taaaaaggtc ttaagaaata |
| 99361 | ccatttggat gtatatattg ctctgaaaag cagtgaaaat catgatacac attatatgaa |
| 99421 | aaattaaact agttttaagt atataggata gtgatttgtc ataattgact gcattccagt |
| 99481 | ttgttaatgt ggtatcaaaa attctcagtt caaaaaaaag aaaaaattct gagtccattt |
| 99541 | tttaatctaa atattgcttt aataattttt attaacaatt ttggagctac tgccttagca |
| 99601 | gagtaaagtt tatctaaact aaaattaata aaagtgccat ttaataaccc tccttctgaa |
| 99661 | aaaaacaggc agcaggccaa atttgtcctg tgggttgtac ttgctgaccc ctgccttgga |
| 99721 | gaataaatta tatacaaaga cactagacgc gaaagaatag aaacagttcc cgtttgcttt |
| 99781 | gaaggttgga agtagcacag aatctgtcat tacctttgtt gggaatgttc aaggagagat |
| 99841 | aaggcagttg ctaaaaaagg gaagatttgt ctgaagcaga ggagaggtac agctgtcaag |
| 99901 | tgtgcctgtg cagggaggga gggaagtaag gtgtggagtt tccactagag tcttggggtt |
| 99961 | gtcataacac aaacatgcca tggctggtca tctggaatgc ccttgggaca aaatattatg |
| 100021 | tgttcaaagg gagtagacat ctccctcaga gaaacttgaa gataacattt taaaatgttc |
| 100081 | tattgaatgt gattcatgcg tttcaaagta gagagaaata ggagtaagag gtgcagtgag |
| 100141 | aattctttac ctactccctt tgaccacata gggttgtctg ttataaaaga agttgaaaag |
| 100201 | gagatccgag atcaaatatc cattattgcc tctacaagaa agggaacttt tctgaaagag |
| 100261 | aagtcaactg agtttatatt gtaaggaggc aagagaggga ctgtttccat gtgcaatatt |
| 100321 | aactagcatt attagaagct agtctggctg atgattccga aataaccagc attctgtgaa |
| 100381 | tggatccctg tgacctctgt cccacggttc taatgagcaa caggaccagc tgcttctagt |
| 100441 | gttgccccat cagcagtacc acgaactgca ggcagagaaa cacaacttta gctggacatg |
| 100501 | gaggggtctt gtcacttaca aaagacaaca gaagacacca atggaaatta tttgcattta |
| 100561 | tctcattccc cagttcttgc tgtgagctcc ttttaaaaat gtttgaagtg atagatttag |
| 100621 | gggtttgagg gacaaaatgt tccagttggc ctctgtgtct tgcatctaga atttgatcac |
| 100681 | tgtgtaaaat tgaaggctta tatttctgca caagaggaga ggaaaaggaa gtggttgaaa |
| 100741 | cactgaaact ataggtagtt gatagataaa caaattcaga tatatataaa tttagataga |
| 100801 | tagatagata gattagatag atagatagat agatagatag atagataggt aggtagatca |
| 100861 | tcggtcttgt gtgtgcctta ccttttagag ctaatccaag cactgatata ataccaaagc |
| 100921 | caagtcctta attattccag gaacatcata gaaatgaaat gtagaaaact aataataatc |
| 100981 | atatactttg tatgtgtatg ttttttccca gcacataaag ggtttgtata agctagtggt |
| 101041 | catcaagctg ttaggaagaa aaagcaaaat taaaactaag atcttctcat ttctattcaa |
| 101101 | aacctattgt ctttttttct ttttcatttt tattttgcat tatatactta atgctgtaaa |
| 101161 | actaatgagg attcaatact agcagaaatt cagacagagg ggttcattat tagttgattg |
| 101221 | ttagtaatcc ttatctctga agttattttc attaattttt cttttgtgta ctttttctta |
| 101281 | gtttccagtt ttttaaaaaa gtgtgttttg atgtataatt aaaggaaaca tcatgaaatg |
| 101341 | tctattattt ttaaaaatac tcatggaaaa tgagtaacag catcattgaa tagttaacac |
| 101401 | atgacagcaa aacctgaatg aataattctc agctcggaga cactgagaaa tatttaattt |
| 101461 | ttacatggtg gagttttttc tgttaagcgt aaagagtcac acatgctctt attcatcccc |
| 101521 | ctttatgccg agatattacg tcaacatgtg agaccatttc gtaaaccagt ttgagatatt |
| 101581 | tctggttact ccttattata tagtttctta aagcacagtg tagaagctgg ttagggatta |
| 101641 | attcctttcc ttcccatcct aacagtcatg gctgacacct atataacaat gacaggttaa |
| 101701 | caagagaaga gcataacaaa tatgtttgat cacagtttta cataacatgg gacccttcag |
| 101761 | aataaagacc ccagatacag ggaaagcaat cctttttttc tgagacagag tctcgctctg |
| 101821 | tcacccaggc cggagtgcag aggtgcaatc tcagctcact gcaacctctg ccccccagat |
| 101881 | tcaagtgatt ctcctgcctc agcccctcga gtagctggga ttacagatgt ccgccaccac |
| 101941 | gcccggctaa tttttatgtt tttagtagag actgggtttc tcgatgtcgg ccaggctggt |
| 102001 | ctcaaactcc tgacctcagg tgatccaccc atctcggcct cccaaagtgc tgggattaca |
| 102061 | ggtgtgagcc accacactca gccaaagcaa tccattttta tgcttaggtt caatgaagta |
| 102121 | tgaacagctg tgtagaaata tgattagaca caaagggtat gatctaatgc taacaggctg |
| 102181 | agtggggaaa ctagcccatc ctgcttgttc agatttttct cagcccctct gtgcagcact |
| 102241 | cactcctccc aggcaagagg taggatccct ctggaatgag ggtctgaatt tctttatggc |
| 102301 | caggtgttac acagaaaagc tgggggtggg tagagtaaat gtttaggttt tatggctggc |
| 102361 | cttgaggaaa aggcgttctg gtgtctgatc tgacttggag aagagggatt ctagtgtcta |
| 102421 | aggtttgcct gagggaagaa agagaggaga gacaggaggg cagaaggtct gtttctgagg |
| 102481 | ctgcttctta ggccttcacc ttggggtgtc gtttactgag cctcaacaac agcatccttg |
| 102541 | tttctgtcta attttctctg ggggttttct cacctttacc ttgagaaagt ttttgtggag |
| 102601 | ttttgtgctt tcttattcaa ggccttcatt ttaaatattc ccaacaaaca aagggataca |
| 102661 | gtaaaactca tgttggctaa ttagaaaaag gccagtatga attaaaggaa gactctatat |
| 102721 | tacaaaatat tgttttaaag tatagctgtg tttcttggaa gttcatcatt ttacctgaat |
| 102781 | gtattatatc tcattccaat gaatttgctt taaagacctg ccagtggtgg tttgtaatta |
| 102841 | tagtattaat tttttaaatt caattggggt cacgtaacaa ttttaaaatt tgacacaaat |
| 102901 | tctaatttgt ttaattttct tactttaaat tctttatata aagacaatga aaaactaaat |
| 102961 | tcgatagttg tacaaatcta aatgagaata taacttaaga gttttcttaa gtcattacta |
| 103021 | acactatgga agatctgata gaacacataa gaatatactg agaccatcac agtgcctaca |
| 103081 | cataataagt attcaatata tgtttgtcag gtgagaatat gagttgttat aattcccagc |
| 103141 | attatattaa gggaacaagt agatgtgtga agagagtacc aatagcattt tccattctcc |
| 103201 | caattgtctt cagttattat agcagataag tttttaatcc tatgtttgtg ctagcactag |
| 103261 | catttacagg tttctccctg atatatggcc cattaacaca caataatccg agttcagagt |
| 103321 | aattacttac atcattcaac agaatagatc actgaggcac tgggtgacca gttgactttc |
| 103381 | catgtgatga gacatgatga gaaaacagca aagctgaagg tgctacttgg ctcctctgga |
| 103441 | agccatattt ctgctttctc ctccctacat cactttcttt ttctttaatg gaattctagg |
| 103501 | aagatgtgtg agtgggagaa tctgagtgtg gaaggtgttg caaagatgta tatgttgtca |
| 103561 | aaaattaagg taaactatca taacacataa ataaactatg ttcctaaagc tgcaaacttt |
| 103621 | ttaggtgaga gcaattattc ttatgctcaa ttatttgtta accatcatga tgaaatgtct |
| 103681 | tccagatgca tctttgctta tatcagctta ttaactaaga gtagtcagat tctatttata |
| 103741 | atgtaaaata ttaaaaatac ccaaatctat tcagtccttc atagttccca aagctttcca |
| 103801 | catctatcgt cacatttggc cttctcaata gccctgtgga gtagctagag atgatataat |
| 103861 | tgcagctgga aatgaagaca atattcaaat ggagcgtgtc aaccaattta ctagtacaat |
| 103921 | tttctcctat ttctgagaca cagatgtgaa aaccaaaaaa aaaaagaaaa gcagaatgaa |
| 103981 | tgccaagaaa tagcatgtga ttagaaatgt tgttcatttc ttctttcagt aaaccacaga |
| 104041 | aagataacaa aggctactgc gcccagtaca gaggggaggt gtgtaatgca gtcctggcaa |
| 104101 | aagatgctct tgtttttctc aacacctcct atgcggaccc tgaggaggcc caagagctac |
| 104161 | tggtccacac ggcctggaat gaactgaaag tagtgagccc agtctgccgg ccagctgctg |
| 104221 | aggctttgtt gtgtaaccac atcttccagg agtgcagtcc tggagtagtg cctactccta |
| 104281 | ttcccatttg caggtaaaat ctcaaaaata agtcaaagga aaaattccat ttttctatct |
| 104341 | gcacaacaga aggagttttt gaaaaatgtt gtaatatgca acagtaaaag gaaaagctgg |
| 104401 | ttttcatcca aaataggagg ttaatgcctg ggtgcagtgg ctcatgcctg taatcccagc |
| 104461 | acttcgggag gccgaagtgg gtggatcact tgaggtcagg agtttaacac cagcctgtcc |
| 104521 | aacatggtga aaccccgtct ctactaaaca tacaaaaatt agccaggtgt ggtggtgcac |
| 104581 | ttgtaatccc agctactcgg gaggctgagg caggagaatc acttgaactg gggaggcaga |
| 104641 | ggttgcagtg agccgagatt gcaccactgc actccagcct gagtgttgag actctgtctc |
| 104701 | aaacaaacaa acaaatagga ggtaattaat cagacaatat ttctaccata taaatcacta |
| 104761 | aagataacct aaaatacaag tacttagaac ctctagaaaa tatcataaat ggtgtaatat |
| 104821 | tccattactc ttgttattgt tcacattatt atcatatcca taaatatccc agatttctat |
| 104881 | cagatgaaag gagaaatctt aaacatggac ctcattagta agccaagata gagaaaataa |
| 104941 | aaactgtaca ttatttgtca ataaatgaga tcatgccaag catctctgaa tgttatttta |
| 105001 | aagtacattc catccaaaat tctaagttct gatgctgttg cctcatttgg gtggttatag |
| 105061 | gaaggaatta gattataaat agatattttg ttaaactccc atacttttta aatattgaaa |
| 105121 | ttgataaatt tatgttcaat agaattatct tcatctttaa ttacttctta tgtagaaaaa |
| 105181 | aaagaatcta gtttttgagc taaatagaac aaagttcaaa tactgttagt tctgctttct |
| 105241 | cgatgtgtgt ggtcttggct aaacttcagt ttcctcatct gcacggtaga attgagagga |
| 105301 | ttaaaggaaa aatcacactc atggaatatg tcagaaacta gatagacatt gttattccta |
| 105361 | aattttctgt tgaggaaacc ccatgacaac aaccaacaca gcacatttcc acttctattt |
| 105421 | tgataaaacc agaaactatt taaatattgc aataatttga ggacactctc tatttagcat |
| 105481 | tcaataacaa cagtatggtc attaaattag agttttgtat tcctcaattt actgcaaagt |
| 105541 | ttgagggttt ggagacacta taagagtaat ataatttgtt tattcatcta agaagattgt |
| 105601 | attgtgatca ggcatgttat ctaaaaatct ctagggactt cagcattgac agaataagaa |
| 105661 | ggggaaagag aaggagagaa aaagtaaaat tactgcaaaa aaaaatggga agagtgggtg |
| 105721 | aggggcagta aaatttgttt taatagaact taagaaatac tgatgctatt tcaactgtcc |
| 105781 | ttgcagttat ttgaaaaaat agtgtctctg ttaattatca cttctttagt ttagatttct |
| 105841 | ctttcttggg cttctttata ggattctcca aaattgttta tttgacgtac tgctcaacag |
| 105901 | tgaaaatcct tttgctagaa agtagcacgt gtataataaa cagaatgtaa ccagcaaaat |
| 105961 | aaagtgacag tagattctat cagcttttcc tttagtacta ctaacataat ttgtggtggc |
| 106021 | cacgggatag ttatcatact tacacagtga cactcaggca aatcagctca acaccatgga |
| 106081 | tagatgcttg agagaaataa taattagatt agaaaaattc tctgtgtgtg tgacagccat |
| 106141 | tgtggttttc tgattacaaa acccacaaga ctccattgta taaaaaaaga acacagtact |
| 106201 | ttcattgcta aaacccagaa agttggtcac tactgaggtg tgtgtttact tatatttact |
| 106261 | tatatctata tttctgtact ctgaattcta tttcatggat ggtttctcta tttttgcatt |
| 106321 | agagactttg ggtttaatta ctgtctttat aattaatttc aatttgtgct acgaatttcg |
| 106381 | caggttttca tcttttaaaa aattattgtt tactcattat ataattattt ttattacatg |
| 106441 | ttgacattgg caaaatgttt ctaaatttca tatttaaaaa taattatata agttatataa |
| 106501 | tacaattata tattaactta acaataatta tcaataataa ttaataatta ttatattaat |
| 106561 | aataaataat acttaatatg atataactaa ttataagcta tataattata tattatagta |
| 106621 | tataacttat ataattgata taatttacaa ttatatatta attatataac atataattaa |
| 106681 | tatataatta taattatagt ttataataat ataattatat attatatagc tcactatata |
| 106741 | attattttta aatataaaat ttagaaacat tttgccaatg tccacatgta aataccattt |
| 106801 | agaatttgat cagaatgatg ctaagtttat acaattaact tagggcacag agctatattt |
| 106861 | acattactga gtattctcat ttaggagtat ggtatgccat tctttttatg tagatcttgt |
| 106921 | attatgcctg ctagtaaaat ttgatgattt ctttcttact tttttttaat taaatttatg |
| 106981 | actaggtgtg ttttatagtc attgctgttg ttgcaagagt attaattata atatagttat |
| 107041 | tacaggaggc tagaatgtcc ataaaacata gagatgcaat taattccctt gttccttatt |
| 107101 | ttaataaaac tgccattaat ttctttagaa tttgtacatt ttagagttga actatgtaat |
| 107161 | tttttggatc atattaatca gatttctaaa aatgtattct tttttttgag gtagttagtt |
| 107221 | gatagaagta taatttatat acaagaaact gtgccccttt taaccatata gttcagagtt |
| 107281 | tgacaaacgt atacaacaat gtaaccacta ccccaatcaa gacatagaat gttttcacca |
| 107341 | cccataaatt tctcttatgc tcatttcctt cttttttttt tttttttttt tttgtccctg |
| 107401 | ggttcaagca attctcctgt ctcagcctcc caagtagctg ggactacagg cacctgtcac |
| 107461 | caggtatttt tgtattttta gtagagatgg ggtttcacct tgttggtcag gctggtctcg |
| 107521 | aactcctgac ctcaggtgat tcacctgcct cggtctccca aagtgctgag attacaggcg |
| 107581 | tgagccaccg cgcccagccc tcttatgctc atttctaatc aacttcccat tatctctaaa |
| 107641 | ggcatcgtcg ttctgatttc tctgacaact agttttgctt gttcctgaat ctcatactca |
| 107701 | tgaaattgta taatatatac tgctttgtgt ctgacttctt ttgttcaaca tgtttttgat |
| 107761 | attcatccac attgctacat gtaacagtgg tttgttcctt ttcattgcag cttttatttg |
| 107821 | tgtggttgag atggaaatag ttatgtgaag aagtaggtag ggtcaagctc cccttcatgc |
| 107881 | tgcctaagaa ttataaagca attaatttga acaactgcag tatcaaaagg aaagttagta |
| 107941 | tagaaatgtt aggccactca agatgacaga ctcatcagcc acaacaaaaa agtcagatac |
| 108001 | cagctttaga cacaaaggat caaccaaata atttattcct gcaatttgta gtttattcca |
| 108061 | attttgtttg gtattatacc tcctaattat tgagaggaat caagtctcat caatcataaa |
| 108121 | tcattaatcc ttgccaacaa atgtgtatta ttttccattt tgttaacgag ttgttactac |
| 108181 | tatataataa agccatttca ttttattata tttatataca acaaccgctt tacagtattc |
| 108241 | tattagattt ttgattgata gtctatggtt cctctgatgg aaaaatatgg tctataaatt |
| 108301 | attatatata gtttatatat aatataaaac tataaatctt aattagattc tactctttct |
| 108361 | taattgaaat ttcagaaatt tttactagaa ttaaaattaa attttatctt catgtgctag |
| 108421 | cacaaaatac atttattaac tttccagcat ttatgtccca gatcagttta catgcctgag |
| 108481 | ccacagatgt tctttctaat cttgaaaata attcaccttt ttgaaagaaa aaaatccctt |
| 108541 | tcattgaaat gttaaattta ttagaaaatc atagttattt cctaaatcta tgtcattcac |
| 108601 | agtatacatt taaatttttt ttctaattcc cattgttatt taaataagta gttttattgg |
| 108661 | gaaaatgtta ttttttaaaa tcctcatcat caaatatttt gttcccattg aagtaagaaa |
| 108721 | atataatcta aaaattttta cattttaata ttttatagag gttttttctc tgatatataa |
| 108781 | taataaaaat tggcatttat aaagacttag tctcttccat taacatacaa aattcatata |
| 108841 | tttatgttca atcctttctg ctcttaaatg agtattagct atttggtcca atagtttaat |
| 108901 | agtgatgcgt caaaattttc acaattgtgt ttccaagaat gttttcttgc aattctgact |
| 108961 | cctgcttttt gcaccaagaa atattaaact aaaattgaat ttactgctga taactggtaa |
| 109021 | gtttgatctt catttttttg ttatatcttt tgaaaaagtc tctaccttta ttcatcaaac |
| 109081 | tagttcagaa gttgtcaacc attttatatt ctacttctac ttgcttgtga tatttttaaa |
| 109141 | agcgtgctat cactcatggt tttctaatta ttaggttggt acaaaagtaa ttgtgttttt |
| 109201 | tttccattaa aagtaatggc aagaaccgca attacttttg ccccaaccta acataagcag |
| 109261 | tcattcaatg atgtgtgcct cttttcatga acgacatgga attttattgt ctaacaaata |
| 109321 | tttattgatc tccttctggg ctccaagtac tacactgagc atttgggata tattaatgaa |
| 109381 | cataataaac aacttatata taactttaga aggtgttaaa tgtcatggaa aaagaaaaaa |
| 109441 | ggaatgcttt aaggaagacc agaaataaca aacggagagg agaaagttgt aactttaaaa |
| 109501 | ataaggtgtt cagggtagac ttaattaagt tagcataatt gtactgaatc ttccacactt |
| 109561 | gttctttgaa actttacctg aaattatcaa ccgtatttgt attgttcaat tatattttat |
| 109621 | ttgcatatgc tcaatttttg gaacattatt cttagctttt acatttgatt tcataatcat |
| 109681 | gtataagtag atacttggag gagcttatac tatgttactt atcattgata ctcattgtca |
| 109741 | ttcatttcaa ataaaatatt tctttttctt aaattcctaa tttaaattca cttcttgttc |
| 109801 | attcatctca tctttactca tctctcattc agctggagta ggtgtttttc acatgtaaaa |
| 109861 | aaaaattctg tcattgtaca tatgtgtttt tgactgggta tttaagtctg tgttaacaca |
| 109921 | agtgttagct tttgcgttag ttctcctcac ttgtgttact tatcctcaaa actgtggact |
| 109981 | ctgaccaatt attttctggc catgttgtaa taaagaggaa gaagaaagca atcctgactt |
| 110041 | ttgttccttt gtaaggaaca ttttcctctc tccagattgt agaaagatta tttctttaat |
| 110101 | tgttgagatt caaaatattt gtaaaattca tttggggata gatttattct cattgttttc |
| 110161 | tttcatgtaa tataaataat ataaaaataa tgggtaatat gccctaaaca ttgtgccctt |
| 110221 | ttcatcttaa aaatgttttc ttataccaca tctttgatta ttttctattt tttatgtgct |
| 110281 | ctggttctct tgctaagaaa cactattatt tgattttgaa cttcttactt gctccacctt |
| 110341 | atcagttatt tcacacatta ttttcatctg tttagccctt cttgcattca gttaggtcct |
| 110401 | ttctttctgc attcaattga atatttaatt caaatttgtt tatttcttgc agtgccattt |
| 110461 | gttttctatt ttcaatgaag attgttaagt gttggtttta ttttttgtgg tttctatttt |
| 110521 | ctgtagttac ttccatgttc aaattatcct actttttagt ctatagttca tcttgatttg |
| 110581 | ttttttcttg tgaaaaactc tgctatagag ggcatgcttt agtgcatggc aatgaaaaca |
| 110641 | aagcaaacca aaagccattc attaatgtgt tcttctgttt agaataaaaa aatttcccag |
| 110701 | gtatttgctt cttctgccta atgtggagta tggagttgtt ttcatatgtc ccatattgat |
| 110761 | ttacacttta actatgctgt ctgcgtgcaa aaaaatgtgc ttatacaacc caggggtgga |
| 110821 | accaagctaa gcaactctcc tcaggtcctt tacctgctca gaagtcttga aagccccatt |
| 110881 | atactccctt ctcccttacg cacaccaaga attataaatc ccattgtttt cttcatataa |
| 110941 | gaatatattt gcatctcatc acattcatat gcacagcctt ttattctatg catactcaat |
| 111001 | tagcacatat tggcaagtta ggaaagtagg agtgtgggaa taagtatgaa aaaagacacc |
| 111061 | attgtggcat atatatacac acacacacac acacacacac actcttacaa tttcctcctc |
| 111121 | tctgaatcct tcagatactg aataaaggac atctgctatt attatgttta atttatttaa |
| 111181 | ttatctttaa tttcctctta ttattaaccc caaatacctt aatttgaaat taaccaggtt |
| 111241 | tttcctatgc agtttgtcct attttctatc aggcttaaac aaatactaat ctgcaaaatt |
| 111301 | ccaagtagtc caattagtgt ttcttcagcc ccttcactca taatacaaca tgtagatgaa |
| 111361 | cataacagct tcatattcaa acagataata ctttggcttt ctcagatgga agcacgcaga |
| 111421 | gcaatgggac aggattccgt gcctcaaagt aagggctgag taaggaccca gatgccctcc |
| 111481 | tataagctgc cagggaggtg gtggtgttct gtttcagagg acaaagcaca cagaggccca |
| 111541 | gactgtgttc ctcgagccct acccattagc cccaagatca tagaaaaatc ttccaagctg |
| 111601 | gaaactggtt gacaatgatc ctgactttca gtaggtgtgt tttttaaact ctgtatacct |
| 111661 | ttttcctcta cctgtgttgt tatttgaata tgaagttggg agaagacata tcagagattc |
| 111721 | ccaatctaat acgttttaaa tttgacgttt ttttccctta gactagcatt aattgtgttt |
| 111781 | cacaacaaaa cgagatagca tttcatctca cgggacagta cactagattt catgaggtct |
| 111841 | catattattt acagtaaaaa gtttctttca taaacgcgct tttttcaaga acaatttacc |
| 111901 | atgatgatgt cttgaaaaca cagaatttag gctctgccac aaatttgctt taacatgtaa |
| 111961 | tgattcatca agttttgttc tccatatact attttagaga gtactgcttg gcagtaaagg |
| 112021 | agctcttctg cgcaaaagaa tggctggtaa tggaagagaa gacccacaga ggactctaca |
| 112081 | gatccgagat gcatttgctg tccgtgccag aatgcagcaa gcttcccagc atgcattggg |
| 112141 | accccacggc ctgtgccaga ctgccacatc taggtaacac agagttctcc caagactttg |
| 112201 | gaggttaaga gaaatttact attttgaaga aactaaggtg tgaacttaaa gcttctaata |
| 112261 | tttctaggca tttctaattt tttttttttg agttctgcct tccttagata cctactagcg |
| 112321 | gaatcctaat gctatcctaa ttgtacagat tagcgtaaag atttatttca tttgctctaa |
| 112381 | aataaatgaa acagcacttt taattactgc ttgacatttt agattctttc atagggaatc |
| 112441 | ttatgagagc ttgtaacatt ttctggagtg tgtagaaaaa tggctaagaa acatcttata |
| 112501 | ttttgtcagc agagtggaat aaaggaataa agtccttttt cttagccaag aaagcatgct |
| 112561 | aaaataagac acatgtttga aaatgctgat ctcacaaaac aaaattgaat tgcttcatgt |
| 112621 | tgctttaatg aactttgttt gtaaggaact atgttgttat ccctaacagc tggtttaatt |
| 112681 | tcaaagaccc aattctttca cattcgaatg caaagaatgt tttccagttg tatattaaaa |
| 112741 | agctgagaga gaacttgcat ttcttagcac tcactctctc acagaatatg tgagatatct |
| 112801 | ggatgctcac ttaaaacaaa tttttatcct ttccccttca gattataaca aagaaaacct |
| 112861 | aaaaagtaag taattgtgtt tgtgcccttg aaaccttatg tgtatgtaat tggattcatg |
| 112921 | catgtgtgtt tacacttata tttcctgatg cccagaacag aatgtacagc cgctctcaaa |
| 112981 | gtcttctcac catactcatc cagggtataa catgagggaa aatcatggaa ggtctcagtt |
| 113041 | tataactttt aattagaccg tagagagtac catatttagt gagtcataca aatgctatct |
| 113101 | cccacctcaa ggtttttagt ctgttttatt tcttatatgg ctataagtaa gccaaattta |
| 113161 | aaatgtcacc ttttgatttt atattcttga ttcagtataa tctaattatg tatagatgaa |
| 113221 | acccaacatt tatttcttca ttgagccaat tcatgatctt catttgtgac aatagatccc |
| 113281 | tcttttggaa gttaggtatt tttttaagac agctgatttc cagtataaag tttataggat |
| 113341 | caaatcaaat ggctttaaaa aggtcatcac aaaaatctct gggaaagaat tcaagaaggt |
| 113401 | tgcttgaaaa gggatgtcgt acaaaataac ttgtatcaga tagtgcttta acctgtcaga |
| 113461 | atgcagaaat cctcaggatg aaattagaaa agcttttcat gtcaactaac ttcctctggt |
| 113521 | ctcagcgttc taaggcttaa atcacactta cttcctgtaa tacatattcc agccctaatc |
| 113581 | tcctctgcat ttgactaggt gccaggactt gattttacca gcttgcttaa aggaccaaca |
| 113641 | cttaaaagta ttaaaagcaa gaatgctact ggtcctagtt gaattactca gaggtattaa |
| 113701 | acaagaagag ttatggaaac cttaaaaatc tattattctg gtagcctgtc aaattgttat |
| 113761 | tcccaaatcg aaggaataaa aaaaaatttc aattctttgt tatatgagca gccaaagttt |
| 113821 | actttgattg gactttgaat tgattactgt gctctttgtt atccatcgat tgctcactca |
| 113881 | tccattcaaa cattcattga gggtttgctg tgtggaagga ccagaccatt catcttaaat |
| 113941 | tagacaggaa caatacacaa aaaagcatcc acatttctgc ctttcacact aattataaag |
| 114001 | gaattttgag cagcacaaac tagctacaat aataaatttt ctagtcttta aaaaacagct |
| 114061 | ttatgaaatc ttcgatacat gtaagaaaat gagctcctaa tgtcatgaac taaccattta |
| 114121 | aactgaaagt agggcaggga atatctgaaa gtatgctcct tgagttaagg caattattat |
| 114181 | taaaagagtt ggctttagca ggtcaatagt ggagcaaaga aaacatcctt attaggcagt |
| 114241 | gtcccatatt ttataggggt gtgtaaagac cttaagtatt ttccaaagag tgtcttgatt |
| 114301 | tctgagtaaa ctctaaagaa tatcaccagc tcatttggag gggtcacaaa agagctgtta |
| 114361 | atcatttgtt tatctttgca ttattttctt cttccttgag catagggact agaatttcag |
| 114421 | tagtttttct tttttataat ttctttggct gaagtgaaac atctttttga gcaggcaatg |
| 114481 | cctttgtgct atttacatta tttgtaagca aggattacac tattcctctc ttccgtgaga |
| 114541 | cttcacatac tttattacag aaacactcag cagtgaatct gtcttctatt tcagagcttc |
| 114601 | tgtggagaaa aggttaattt gtcttaatca ttgaaataat tacttttctc ttgaattctc |
| 114661 | tttttccttc catctttgct ctttctatac atatacatgg agaataaagg acccaatgaa |
| 114721 | tgtacccaaa tccaaactga ctagtgtgat acatacatta tctctcttaa tactcccaat |
| 114781 | agttctacaa aattaagtac tatgaacgtc ctccatttta cttacaaccc aacatgagaa |
| 114841 | agagagatta agcaatgtgt ccaattttac ccatctagta aggtgataag ccagaatttg |
| 114901 | aactgaaatc tatctgattc tggctctgtc ttcagagcca gaatgttata cctgatgcat |
| 114961 | ttctgttatt actaatgttt cttaaaaaat tccaagacct catgtgtaat aaagcttaac |
| 115021 | tgattgttta actgattcat tcttgaataa attccactac cttttcacat ttgttataat |
| 115081 | tgattattct taagaagctc taattgataa aaattttatt gttcacaatt ttaaatcacc |
| 115141 | caggccactt gatgatttga ctttttgtta acatttaaat gaatacatgc tttgtgtccc |
| 115201 | agaccatttt tccataatga tcagctacta ttttgcccac gaatgaaatc atgaagcgtc |
| 115261 | agtgtctctg tgtgtgtgtg tgtgtgtgtg tgtctgtgta aaactcatga attatttcat |
| 115321 | aatttgaaat gtggtttggg taatttttgt ggtaatatta aagttacaag ttcttagaaa |
| 115381 | attggagttt aaagtaactt taacagttat ttaatctaaa gtctcccaca atgaaagaat |
| 115441 | cttttctaga gcatctctga cagaaacatt ccattttccc tcctgaattt atttctcaat |
| 115501 | tcctttgtag aatacattaa ttatttcaac aatttagcta tgtagaacta aattctggtt |
| 115561 | ctctagaact gtcaacaact gatcttcact ctatttcttt gatttattta taatctgtat |
| 115621 | gatagtcctt aaaatatttg gagacaggca ctcaatctct ctgtagtctt ctcttcttca |
| 115681 | cattaaatat ctccaatttt taactttttc ttttgcaact gacttccaaa atcctcacct |
| 115741 | ccatgtttac tttttactgg ctgccacaag cttgagaata tccagaactg aacacaaagc |
| 115801 | acagaacatg atctgatcag aattgcaatg caaccatatt ttctgtgata aggccttgtt |
| 115861 | cttttatgag tatagattaa actcatgcta gctgtctaat aatttcatga cattaatagc |
| 115921 | ccttctatac acatatgggt ttactgattt ccttgatttt ctaataaatt ttctactaga |
| 115981 | ccaatccaat tcataattat ttttattaca ttcttctagc atttataata atttgtactt |
| 116041 | tacatgttta gttactatgt tctatgaggc atagtgatcc aagacacttt tctttttata |
| 116101 | tgaagctctt taccaatgta aaatgtcact cttgtcttgt tttagtgctt ttagctttaa |
| 116161 | atttctagta ttaataatgg taattttttc cttgtattta tgttaaccta gcacctcttt |
| 116221 | gttcctcttt tatttttctt ttcttataaa caacatatac ctgggtttac tccaatcaga |
| 116281 | tagccatttt ctattaatag gaggatttag cctgttcata ttttatatca caactgatat |
| 116341 | actctatttt atccctacta tcttaactgt atattttcta ttcgctttac gtttttgctt |
| 116401 | cttctttttc tttttcttta cttgttactg gttatgtttt ctccactgct aagtgaaagt |
| 116461 | cagacaccaa ttctttctac acatcgcagt gctctattta ttcttcattt ctgcccaccc |
| 116521 | catgggactt tcagaatctc caggagatcc tcagactgct tggatcttct tccttacctc |
| 116581 | ccctggtact cttaggtttc tctttgcaga gtgattctca atattgagtc ttacctagcg |
| 116641 | aattagccaa gactccaccc atcacagccc cttcacatgt atcctgaagt tattttctga |
| 116701 | ttcaattgtt attaagagtc tcctcatgta tttccttaga agggaaagtg tcttgggaga |
| 116761 | cagaatccct gaagccttgt gtgttatcaa attgttttct tttatttggg tagttgaaaa |
| 116821 | gtgtattaac tgggtatgga actcctgagt gacatttctt tgggtggttt atagatgtga |
| 116881 | tttcactctt ttctagcttc tagtgttgga agcaggtagt ctggtgtgaa tttatttttt |
| 116941 | tcctgtaagt aatttattct ttctatctag gcagaaactt tttgattttt ccctttatcc |
| 117001 | gtgatatctg tatgataaaa attgtttaat catgtgctat tgaacatctc tttccaactc |
| 117061 | catgataggg cttcatgttg ggagcttgag ataagctatg atgggaatct ttatcccatt |
| 117121 | gaaatcagga tggtagacag acctttctaa tgcaaaaagt tgatcatgct attccacctc |
| 117181 | gtaaaacact cctataagac cctccatttg ctgcagggca tagtccaaat tacttaaatt |
| 117241 | ggtatacaag gacttttatc actaaacctg atatctgcag ctttacctat agatactccc |
| 117301 | caactccaac aatgaaactt ctaggtgcac ttttagaaag gcatcacatc tgcttagttc |
| 117361 | agatcttggt atatgctatt acttctttat agaatactat ctctctaccc tgactccatt |
| 117421 | ctactctctt aattccccca tcccacacac acgtttgtgt gtgttttttt gttttgtttt |
| 117481 | gttttgtttt gttttgtttt gtttgagacg gagtctcact ctgtcgccca ggctggagtg |
| 117541 | cagtggcaca atcttggttc actgcaagct ccgcctcccg ggttcacgcc atcctcttgc |
| 117601 | ctcagcctcc cgagtagctg ggactacagg cgcccaccac catgcccagc taattttttg |
| 117661 | tatttttagt agagatgggg attcaccgtg ttagccagga tggtctcgat ctcctgacct |
| 117721 | cgtgattcac ccgccttagc ctcccaaagt gctgggatta caggcgtgag ctactgctcc |
| 117781 | cagcccacac cttttcctta gtctgttcag gttgctataa caaaatgcca cagactgggt |
| 117841 | ggcttataca cagaaattta tttctcacag ttctggagtc caagaagtcc aagatcaagg |
| 117901 | tgccagcaga tttggtatct ggtgaggacc cattcctcat agacatagct gtcttctccc |
| 117961 | tgtgtcctca catggcagaa cgggcgaggg agctttcttg gacctctttt ataagggcat |
| 118021 | taattccatt tatcaggact ctacccttat tcatttcaac acatgaatgt gggggaaaga |
| 118081 | cacaaagact cagatcatca taaccttcaa ctggttactg gctactcacc atttatgttt |
| 118141 | aacttacagt tactcactct agaaaacctt ctcagtatgt ctaaacctga gttagtgcca |
| 118201 | gccctgaacg agactcacaa gcatcgtgtt cttactactc ctataatgct tcttatattt |
| 118261 | tgttttattt ctccatttat tcacttgtgt ctcatgacaa gcaaagactt ctcatgttca |
| 118321 | ccacagtgtt ctggatacct aacagtcact attgatttag tgaatgcatt tacatatgga |
| 118381 | tgaatgagtg tataaactcc cgtagtgttc agaggaaaag aatcaagaca gaaaattggt |
| 118441 | gagagaaaag ataaaaagga aaaagaatga cgattcaaag tgatccttta gacccaaaga |
| 118501 | gatcattgat gtagttcaag ggaagatcag atcaaatcag aagcagtcat caaggaataa |
| 118561 | caaaacaaaa ctatcttgaa ggaaagaaac atttaaatcc ataaagcaaa attattgttc |
| 118621 | caactatgtc atttcttgca ttttaaaaaa actcactaaa tcatcataga tttttctttt |
| 118681 | aacatgacag taaattctac tttctgattc tttttagaaa aactaacctt atatttacca |
| 118741 | gtgaaattac actaccaact ttactaatta cattatattc cctcacatgt tatatatggt |
| 118801 | gtgagatgct ctgtaaactc caaatacaaa aatagttatt tctccctatc ttttaataat |
| 118861 | tgttccaact ttattattag cagaaagcaa agctcgtcaa tttaagccac cccctaccac |
| 118921 | caccacccac ccatcttaga tcgagaaaac ttgggatgac tcagtccaaa gagaatcaga |
| 118981 | ctagctttca agtcagtcag agaggaacct ggtcagtctg ttgagtccac cactcctgtg |
| 119041 | gtgccggatg aggcatgctg tcactgatct tgcgcagatg ttttattgct gtgctgatgt |
| 119101 | tcttccagtt ccaggtgcct gcctgagtca cagtggtgac caggcctggc atccgcaggc |
| 119161 | ttcagaatca cttgcttagc cattggtact cagggcataa tcactgggtc cacttcatgt |
| 119221 | atgactaaga agtcatacat gagcgtctct tgtagcctcc aattttgcaa gtgccctatt |
| 119281 | tcaaatgctt taggcatcta ctgccataaa ggttttattt tgactttgta atatgttata |
| 119341 | ttaatatgtc cccttctatt aaatcattat tctctgacta tgatgtagtc agttgctaat |
| 119401 | atttcgttta ggatcttcta aacttaggtt gtataatcta tgtatgctct tttcttctgt |
| 119461 | gctaacattg ccaggttgtg atattggtat tataataatt tctaaaataa tttgaaagaa |
| 119521 | ttttctctta ctggaacata ttataaatat ggaaatttta tctctttcaa gactaaaaga |
| 119581 | atgtctttta taatgtgatg acttaaaaat ctagtttcct gcctatggta atatttatat |
| 119641 | tttctaatgc ttcaaatttg gggattaata tgttcctatc attttcctta aggtgtgaaa |
| 119701 | tataataatt acacattatt ttaattaact ttgaatctgt tataactttt ttatcatttc |
| 119761 | taattttctg tttgtacctt ttctctttaa ttaaaattcc attgattaga ctactatatg |
| 119821 | aatatttata aaaccaacta ttgatctttt aattctatca tttgtgttac gtgtattcta |
| 119881 | acttattaac ttctgcttgt gtatggctgt tactttatta cctattatct tttgttttct |
| 119941 | gtagttattg tttcttaccc aaagtcttgg gttaaatgtt cagttcattt actttcattc |
| 120001 | ttttttatct agtggaagga gataacacta tgactttatc tttgagcaca actttggcta |
| 120061 | aataaaatac acactattaa gaggcttgtg tctaatctat aactatagtt actctctagt |
| 120121 | ttttgatcaa aaagtcattg atttttttct ttttaaaaat gtcccattgg attttattta |
| 120181 | atctattatt aattgcactt tggccattaa gtagatatgc ataatttctg cctttaaaat |
| 120241 | ttattgagat taatccacaa atatttgaaa ggaaggttta ttctctattt ttgggtacaa |
| 120301 | aggtcattct tagctattaa aacattgttg ctgattattt taaaatactc tgtatccctg |
| 120361 | tttttgtctt tactcaatct gttaagagga tttaaatgtg attaataagg ttcttttaat |
| 120421 | acatatacaa tggactttgt actctacaaa tagtggtatg agtatggcat cttttggttt |
| 120481 | ccaacagtaa tgctgatttt actttataca tatcacatcg gccttttaaa ttgaagtttg |
| 120541 | acaggagttc agtttttaga tgaattaact tctcatttgt ctcctaatat tttcattttt |
| 120601 | catcttcttt catgattttt cttctgtttg taaactgaca gtaaatgtat ataaaagctc |
| 120661 | agctctagac aatcctgatg tcaagtgtaa ttatgtgaaa atattttatt tgataatttc |
| 120721 | catgatttat gtcaggtcat agataatatg attcagtata aatggcacca gcccaagatc |
| 120781 | tacatttatc tcagaaaaaa aaaacctatt tcctctctct gagtctcatc tttaaaattc |
| 120841 | tattattcta taagctggat ttacttacaa ctagacctta ctgaacttta taaatatata |
| 120901 | accagggaga acctgataca taaatatatt attatcatga aatttatttt actgattgct |
| 120961 | taaatacaaa aaattgcttc ttaaaggttt gagaatgaat gaaataatta ttctttactt |
| 121021 | acagcattcc caccaatgac gtcctcaaag ccaagtgtgg acattccaaa tctgccttcc |
| 121081 | tcctcctctt cttccttctc tgtctcacct acatactcca tgactgtaat aatctccatc |
| 121141 | atgtccagct ttgcaatatt tgtgcttctt accataacta ctctctattg ctgccgaaga |
| 121201 | agaaaacaat ggaaaaataa gaaaaggtga gattctagtt tcagctaacc atgtgtagta |
| 121261 | atattttaaa gccttgtgtt ttatggagtt ggtatgacgt gaggaatata tatgacactg |
| 121321 | caactctcta aatatatccg tagccctgct ttaaaattaa gcacccccat catgatcttt |
| 121381 | acaaatgtca atgacttttt ggcatgggat caaatcaaat gccctagggt tactgtttca |
| 121441 | caaaaggtaa aatctaaaca ataatttgga ggaaaatggc tattcctgct ccttgtcttc |
| 121501 | tcctcaggcc tgttgccatt gctggcttgg agagctaaac atttctgaga aaatgtgcct |
| 121561 | tttgactcta gaccaaacaa aatattttat tgctctcaaa acgaaggatc tttggctgcc |
| 121621 | ttagtataac ttgttttcat tactcttaaa tgccatattt cttagaatct aagtctaagt |
| 121681 | acaaagatct aacctgcttc tgagctcatt cctgatcttg cctgtcttgc ctgcagagaa |
| 121741 | tcagcagcag taaccctcac cacactgcct tctgagctct tactagatag acttcatccc |
| 121801 | aaccccatgt accagaggat gccgctcctt ctgaacccca aattgctcag cctggagtat |
| 121861 | ccaaggaata acattgaata tgtgagagac atcggagagg gagcgtttgg aagggtgttt |
| 121921 | caagcaaggt aaagttacct atggaaaaaa aaactccatt gaaatatgtt atgtctgaaa |
| 121981 | agctaccaaa ctattagctt ggtatatata taatattagc tttatatata tatacacaca |
| 122041 | catatatata tatatatcaa gctaatgata tagtacatat atatgtacac tgtgtgtgta |
| 122101 | tatatatata cacacacaca tatacatata tacacacagt gtaatataca gtattatata |
| 122161 | gtattaatac aatattatat atgtattata tggtaataat atactatata aaatagtttt |
| 122221 | tatcaagtta gttaattctg gccaggtgcg gtggctcgtg cctgtaatcc cagcactttg |
| 122281 | ggaggctgag gcgggaggat caccttagtc aggaattcaa gaccagcctg gccaacatgg |
| 122341 | cgaaaccccg tctctactaa aaatacaaaa ttgagccagg catggtggtg cacaactgta |
| 122401 | gtcctagcta ctcgggagtc tgaggcagaa gaattgcttg aagccggaag acagaggttg |
| 122461 | cagtgagcca agatcgcgcc actgcactcc agcctgggtg acagagcaag actctgtctc |
| 122521 | aaaaaaaaaa aaaaaaagaa aagaaaaact agttaattct ttacatcatt aaccttattt |
| 122581 | gtattgcata atacacttca aatcttaatt tcccctcttt gctggaaaac agtagcctat |
| 122641 | atggagataa tatattttac tgcacttaaa ataggtaatt tattggagat gtaaatgtgt |
| 122701 | cttcacttct acgattttta aattataaga aattagtaat atagttcaat aatatttcct |
| 122761 | tttcaggaaa gaatttacca tatattttta atcataattt cttacattgt tttaaataac |
| 122821 | tccttacatt ataatataat gtatagatat taagtagatg ttcatgaaat gacaaacaga |
| 122881 | aaaggaataa aaaggcaaaa atttataaaa aatgagctca agaaaggagt atgtattagg |
| 122941 | gccaatatta gcctatgtat cacctttgtg cgaattggaa taaaataccc tttctgctgc |
| 123001 | atggatatgg ctccacagac acacagctga gccagagaat atgaatttgg ttttaatctc |
| 123061 | caactggcac ttcggcctgg tgctcttggg caagacacaa cttaatctcc acccatggtg |
| 123121 | gccctggcgt agaggttaca tgaatagtga aaatctcaca caaacatgtc ttggaaacca |
| 123181 | aaatttgcaa ggcaccactt taggctcaat ggagggtaca aagatgagta agccatagtt |
| 123241 | cctacgtcat ggaatttaga ttctggacaa agattaaaag gttaggatgt ggctgggcgc |
| 123301 | gatggctcac acctgtaatc cccgcagttt gggaggccga ggtggtcata tcacgaggtc |
| 123361 | aggagattga gaccatcctg gctaacacag tgaaaccctg tctctactaa aaatacaaaa |
| 123421 | aattagccag gtgtggtggc gggtgcctgt agacccagct actcaggagg ctgaggcagg |
| 123481 | agaatggtgt gaacccagta tgtggagctt gcagtgagcc tagatcatgc cactgcactc |
| 123541 | cagcctggat gacagagtga gactctgtct caaaaaaaaa aaaaaaaaaa aaggttagga |
| 123601 | tggcaatgat aatcctattg ggggtgtata cttcagaatt cctataattt gtaaataatg |
| 123661 | catctttcaa acttcattgt ttgaaattgt tgaaaataac accagtgatt tttattagtt |
| 123721 | gacatcaagg agttttaagt atgttgtctt tctttgattc tgtggctctg ggaaaggctg |
| 123781 | cgtgagactt aaccagcctt ttacacaggg gaggtgggag gatatgtata aatgtgggta |
| 123841 | ggtataaaga tatgatgtcc atgatctttg gtttcttttt caataaatgt atctctcagg |
| 123901 | gcaccaggct tacttcccta tgaacctttc actatggtgg cagtaaagat gctcaaagaa |
| 123961 | gaagcctcgg cagatatgca agcggacttt cagagggagg cagccctcat ggcagaattt |
| 124021 | gacaacccta acattgtgaa gctattaggt atgaaaatac aagtgaggat atgtatttca |
| 124081 | tcagaaaaca gaggctttcc aagtttttct ccccttgttc gtgctttttc cttttctcct |
| 124141 | tgatcagtga gacgtttcag ttccttttca aatttagctt cttcaccttg gtctatccca |
| 124201 | accctatagg acattcatgg cttaatggat tatccctctt taaattcatc acttcaattt |
| 124261 | gtctctgtct ttgctgcctc ctatttataa aaatgtttaa aatgtcccca ttgtaaaaaa |
| 124321 | ttaatactgc ttctccatca caaaatttat caatattgaa aagtctatat attttactta |
| 124381 | ttttgtttct cctctttctt ccctaactag aatgtcatga gcagcgattt gtatcttttc |
| 124441 | tgttcattgt tgagttccag tttattccca gattcataaa ttcaacacaa attttacaaa |
| 124501 | tatttagttt gtattaatta cacagcaatt aataaaactt ttcaaaaatc tgtgctctcg |
| 124561 | gctgggagca gtggctcatg cctataatct cagcacttta ggaggccgag gcaggcagat |
| 124621 | cacttgaggt caggagtttg acaccagcct ggcgaacaag gtgaaatccc atctctacta |
| 124681 | aaaatagaaa aattagctgg gtgtggtggt gtgggcctgt aatcctagct actcgggagg |
| 124741 | ctgaggcatg agaatcgctt acaccggggg ggcagaggtt gcagtaagcc aagattgcac |
| 124801 | cactgcacca cagcctgggt gacagagtga gattccatct ttaaaaaagg aagaaaaaaa |
| 124861 | aaatctgtgc tctcatagag catacattca ctctatatga ggagacagat aataaaaaaa |
| 124921 | aaacaaaaat atgggatgct ggatattaag ttctacagac tttttaaagt gttaaagcag |
| 124981 | agaagttgga atgtgagcta caaagtttga agacttcagt gaaaatatta catttatata |
| 125041 | aagacctggc agaggcaagg gaataacagt gtggatatct tgggaaagaa cattccaaaa |
| 125101 | agaatgagaa gcatttgcaa aggtactgtg gcaagagcag atgtggcatg cttgaggaat |
| 125161 | agcaaggaga tgaatgtggc tggagcagag tgaaagatgg ggagagttat caaagatgag |
| 125221 | atcagagagg taatggggga ggtaggcagg tcctgtcagg ccttattaaa aacattggct |
| 125281 | tttaatctaa gttgggaaac aaatggagag tttcagccaa gacatgacat gacacaactc |
| 125341 | acattttttc agatcttctc tggctgcttt cttgagcatt gcatgaaggg aagcaatgaa |
| 125401 | atcaggaaga cagtaaacag gtattgcaat gatgcagcaa gagaagatga tggcatgggc |
| 125461 | caggtggtgg tagtgaagat cgtgagaagt gattctcttc catgtgtaat gtgaaagtag |
| 125521 | agccaaataa tttgctgaca gaccaaatat gagatgtgca aaaaagggaa aagtcaaaga |
| 125581 | tagtatcgaa gtatttggct gtgcagatga gtgaatggag gtgccattgg ctgagttgag |
| 125641 | gaatactcag aaggaacagg ttcctttagt ggagagagtg ggaattcatt taaagatgtg |
| 125701 | gtttttgtcc aggcactgtg gctcacgcct gtaatttcaa cactttgtga ggccgaggcg |
| 125761 | agcagatcac ctgaggtcag gaattcgaga ctagcctggc caacatggtg aaaccccatc |
| 125821 | tctactaaaa atacaaaaat tagccgggtg tggtggcaca cgcttgtaat cccagctgct |
| 125881 | tgagaggctg aagcaggaga attgcttgaa cctgggaggc ggaggttgca gtgagccgag |
| 125941 | attgtgccat tgcactccag cctgtgtgac agagtgagac tccatctggg aaaaaaaaaa |
| 126001 | gacgtgtttt ttggcgatgc ttattcaaca tccacatgat ccacatgaag ttaaggatgc |
| 126061 | atgtggatat ggaggcctag agttctggta aggatgatct ggttagacat gaatccaagg |
| 126121 | ctattgcagt acacagagta tttaaagcca tgaggttgag taagacacct aagtatggag |
| 126181 | aaagcaagcc agaacagaga agaggtccaa ggaccaaggc ctggagcatt ccagtattta |
| 126241 | gtgtgagaaa gaggagatga acaagaaaaa caaactaaaa acgagcaacc aatgaaatag |
| 126301 | aagtaaatca agagagtgtg gtgtcctgga tgtcaaatga agtattttaa ataggtgaaa |
| 126361 | gtgagcatgt catatgcatt aagtcaagtg agaggaggac tataaagtga ccttgaattt |
| 126421 | gtaaacatgg atgttcactg gtaaccccga gaagagaggt ttcttaggaa tgttgggggc |
| 126481 | agaggaaatg taagtgagct caaaagaaga ggaagagaaa atctggagaa aaagtataaa |
| 126541 | caacttgttt tgccataaag gtgaaccaag gaacatggat agagctgaag ggggcagaaa |
| 126601 | gaagcatgct tcctttcatg tggaagtaat agcacatttg taaattaatg agaatgatcc |
| 126661 | cgtaaagaga aaaattgatg acagggttgg caaatagtgg aagtaacatc tttgaacagt |
| 126721 | gagagggaat ggaatcttgg cactgttgga tggattggcc tttgtaaaga gaatgctcag |
| 126781 | atcaaccata gtaataagag agaattggct ggagattgta gccagtctct tctgattgat |
| 126841 | ttagttttct ctgtgaggta ggaatcaaga tctcatctga gggtgaggaa tgggagaaga |
| 126901 | gttgaagatt tgaggaaaga gaagaaataa attgtcatct aagaaaatcc atttttacat |
| 126961 | aaaatataaa atggactaga gaaacataat atgattggca agaacattaa aagcttactt |
| 127021 | gaaattagag attacacatt taacatgaga ctggtcagca tggttgtgta tttatccaaa |
| 127081 | tgctgccagc tgggcataga ctcgttaaga ggagttaaat tttaccagaa ttagcatttt |
| 127141 | gccacacaag taagttgaag tcaaattgag gcaaataaga gaggtgtatg caaaggagtg |
| 127201 | attattatga ttgatcaagg aacttaagct gtttaaggaa ggaagacaaa acgtgaagaa |
| 127261 | agtagggatc attaaaaggg ggtaggatca atagattaaa agtttcaggg ggaggagcca |
| 127321 | agatggccga ataggaacag ctccggtcta cagctcccag cgtgagcgac gcagaagacg |
| 127381 | ggtgatttct gcatttccat ctgaggtacc gggttcatct cactagggag tgccagacag |
| 127441 | tgggcgcagg ccagtgtgtg tgcgcaccgt gcgcgagccg aagcagggcg aggcattgcc |
| 127501 | tcacctggga agcgcaaggg gtcagggagt tccctttctg agtcaaagaa aggggtgacg |
| 127561 | gacgcacctg gaaaatcggg tcactcccac ccgaatattg cgcttttcag accggcttaa |
| 127621 | gaaacggcgc accacgagac tatatcccac acctggctcg gagggtccta cgcccacgga |
| 127681 | atctcgctga ttgctagcac agcagtctga gaccaaactg caaggcggca acggggctgg |
| 127741 | gggaggggcg cccgccattg cccaggcttg cttaggtaaa caaagcagcc aggaagctcg |
| 127801 | aactgggtgg agcccaccac agctcaagga ggcctgcctg cctctgtagg ctccacctct |
| 127861 | gggggcaggg cacagacaaa caaaaagaca gcagtaacct ctgcagactt aagtgtccct |
| 127921 | gtctgacagc tttgaagaga gcagtggttc tcccagcacg cagctggaga tctgagaacg |
| 127981 | ggcagactgc ctcctcaagt gggtccctga cccctgaccc ccgagcagcc taactgggag |
| 128041 | gcacccccca gcaggggcac actgacacct cacacagcag ggtattccaa cagacctgca |
| 128101 | gctgagggtc ctgtctgtta gaaggaaaac taacaaccag aaaggacatc tacaccgaaa |
| 128161 | acccatctgt acatcaccat catcaaagac caaaagtaga taaaaccaca aagatgggga |
| 128221 | aaaaacagaa cagaaaaact ggaaactcta aaacgcagag cgcctctcct cctccaaagg |
| 128281 | aacgcagttc ctcaccagca acagaacaaa gctggatgga gaatgatttt gacgagctga |
| 128341 | gagaagaagg cttcagacga tcaaattact ctgagctacg ggaggacatt caaaccaaag |
| 128401 | gcaaagaagt tgaaaacttt gaaaaaaatt tagaagaatg tataactaga ataaccaata |
| 128461 | cagagaagtg cttaaaggag ctgatggagc tgaaaaccaa ggctcgagaa ctacgtgaag |
| 128521 | aatgcagaag cctcaggagc cgatgcgatc aactggaaga aagggtatca gcaatggaag |
| 128581 | atgaaatgaa tgaaatgaag cgagaaggga agtttagaga aaaaagaata aaaagaaatg |
| 128641 | agcaaagcct ccaagaaata tgggactatg tgaaaagacc aaatctacgt ctgattggtg |
| 128701 | tacctgaaag tgatgtggag aatggaacca agttggaaaa cactctgcag gatattatcc |
| 128761 | aggagaactt ccccaatcta gcaaggcagg ccaacgttca gattcaggaa atacagagaa |
| 128821 | tgccacaaag atactcctcg agaagagcaa ctccaagaca cataattgtc agattcacca |
| 128881 | aagttgaaat gaaggaaaaa atgttaaggg cagccagaga gaaaggtcgg gttaccctca |
| 128941 | aaggaaagcc catcagacta acagcggatc tctcggcaga aaccctacaa gccagaagag |
| 129001 | agtgggggcc aatattcaac attcttaaag aaaagaattt tcaacccaga atttcatatc |
| 129061 | cagccaaact aagcttcata agtgaaggag aaataaaata ctttatagac aagcaaatgc |
| 129121 | tgagagattt tgtcaccacc aggcctgccc taaaagagct cctgaaggaa gcactaaaca |
| 129181 | tggaaaggaa caaccggtac cagccgctgc aaaatcatgc caaaatgtaa agaccatcga |
| 129241 | gactaggaag aaactgcatc aactaatgag caaaatcacc agctaacatc ataatgacag |
| 129301 | gatcaaattc acacataaca atattaactt taaatataaa tggactaaat tctgcaatta |
| 129361 | aaagacacag actggcaagt tggataaaga gtcaagaccc atcagtgtgc tgtattcagg |
| 129421 | aaacccatct catgtgcaga gacacacata ggctcaaaat aaaaggatgg aggaagatct |
| 129481 | accaagccaa tggaaaacaa aaaaaggcag gggttgcaat cctagtctct gataaaacag |
| 129541 | actttaaacc aacaaagatc aaaagagaca aagaaggcca ttacataatg gtaaagggat |
| 129601 | caattcaaca agaggagcta actatcctaa atatttatgc acccaataca ggagcaccca |
| 129661 | gattcataaa gcaagtcctg agtgacctac aaagagactt agactcccac acattaataa |
| 129721 | tgggagactt taacacccca ctgtcaacat tagacagatc aacaagacag aaagtcaaca |
| 129781 | aggataccca ggaattgaac tcagctctgc accaagcaga cctaatagac atctacagaa |
| 129841 | ctctccaccc caaatcaaca gaatatacat ttttttcagc accacaccac acctattcca |
| 129901 | aaattgacca catagttgga agtaaagctc tcctcagcaa atgtaaaaga acagaaatta |
| 129961 | taacaaacta tctctcagac cacagtgcaa tcaaactaga actcaggatt aagaatctca |
| 130021 | ctcaaagcca ctcaactaca tggaaactga acaacctgct cctgaatgac tactgggtat |
| 130081 | agaacgaaat gaaggcagaa ataaagatgt tctttgaaac caacgagaac aaagacacca |
| 130141 | cataccagaa tctctgggac gcattcaaag cagtgtgtag agggaaattt atagcactaa |
| 130201 | atgcctacaa gagaaagcag gaaagatcca aaattgacac cctaacatca caattaaaag |
| 130261 | aactagaaaa gcaagagcaa acacattcaa aagctagcag aaggcaagaa ataactaaaa |
| 130321 | tcagagcaga actgaaggaa atagagacac aaaaaaccct tcaaaaaatc aatgaatcca |
| 130381 | ggagctggtt ttttgaaagg atcaacaaaa ttgatagacc gctagcaaga ctaataaaga |
| 130441 | aaaaaagaga gaagaatcaa atagacacaa taaaaaatga taaaggggat atcaccaccg |
| 130501 | atcccacaga aatacaaact accatcagag aatactacaa acacctctac gcaaataaac |
| 130561 | tagaaaatct agaagaaatg gatacattcc tcgacacata cactctccca agactaaacc |
| 130621 | aggaagaagt tgaatctctg aatagaccaa taacaggctc tgaaattgtg gcaataatca |
| 130681 | atagtttacc aaccaaaaag agtccaggac cagatggatt cacagctgaa ttctaccaga |
| 130741 | ggtacaagga ggaactggta ccattccttc tgaaactatt ccaatcaata gaaaaagagg |
| 130801 | gaatcctccc taactcattt tatgaggcca gcatcattct gataccaaag ccgggcagag |
| 130861 | acacaaccaa aaaagagaat tttagaccaa tatccttgat gaacattgat gcaaaaatcc |
| 130921 | tcaataaaat actggcaaac cgaatccagc agcacatcaa aaagcttatc caccatgatc |
| 130981 | aagtgggctt catccctggg atgcaaggct ggttcaatat acgcaaatca ataaatgtaa |
| 131041 | tccagcatat aaacagagcc aaagacaaaa accacatgat tatctcaata gatgcagaaa |
| 131101 | aagcctttga caaaattcaa caacccttca tgctaaaaac tctcaataaa ttaggtattg |
| 131161 | atgggacgta tttcaaaata ataagagcta tctatgacaa acccacagcc aatatcatac |
| 131221 | tgaatgggca aaaactggaa gcattccctt tgaaaattgg cacaagacag ggatgccctc |
| 131281 | tctcaccgct cctattcaac atagtgttgg aagttctggc cagggcaatc aggcaggaga |
| 131341 | aggaaataaa gggtattcaa ttaggaaaag aggaagtcaa attgtccctg tttgcagacg |
| 131401 | acatgattgt ttatctagaa aaccccatcg tctcagccca aaatctcctt aagctgataa |
| 131461 | gcaacttcag caaagtctca ggatacaaaa tcaatgtaca aaaatcacaa gcattcttat |
| 131521 | acaccaacaa cagacaaaca gagagccaaa tcatgagtga acttccattc acaattgctt |
| 131581 | caaagagaat aaaataccta ggaatccaac ttacaaggga tgtgaaggac ctcttcaagg |
| 131641 | agaactacaa accactgctc aaggaaataa aagaggacac aaacaaatgg aagaacattc |
| 131701 | catgctcatg ggtaggaaga atcaatatcg tgaaaatggc catactgccc aaggtaattt |
| 131761 | acagattcaa tgccatcccc atcaagctac caatgacttt cttcacagaa ttggaaaaaa |
| 131821 | ctactttaaa gttcatatgg aaccaaaaaa gagcccgcat cgccaagtca atcctaagcc |
| 131881 | aaaagaacaa agctggaggc atcacactac ctgacttcaa actatactac aaggctacag |
| 131941 | taaccaaaac agcatggtac tggtaccaaa acagagatat agatcaatgg aagagaacag |
| 132001 | agccctcaga aataatgccg catatctaca actatctgat ctttgacaaa cctgagaaaa |
| 132061 | acaagcaatg gggaaaggat tccctattta ataaatggtg ctgggaaaac tggctagcca |
| 132121 | tatgtagaaa gctgaaactg gatcccttcc ttacacctta tacaaaaatc aattcaagat |
| 132181 | ggattaaaga tttaaacgtt agacctaaaa ccataaaaac cctagaagaa aacctaggca |
| 132241 | ttaccattca ggacataggc gtgggcaagg acttcatgtc caaaacacca aaagcaatgg |
| 132301 | caacaaaagc caaaattgac aaatgggatc taattaaact caagagcttc tgcacagcaa |
| 132361 | aagaaactac catcagagtg aacaggcaac ctacaaaatg ggagaaaatt ttcgcaacct |
| 132421 | actcatctga caaagggcta atatccagaa tctacaatga actcaaacaa atttacaaga |
| 132481 | aaaaaacaaa caaccccatc aaaaagtggg cgaaggacat gaacagacac ttctcaaaag |
| 132541 | aagacattta tgcagccaaa aaacacatga agaaatgctc atcatcactg gccatcagag |
| 132601 | aaatgcaaat caaaaccact atgagatatc atctcacacc agttagaatg gcaatcatta |
| 132661 | aaaagtcagg aaacaacagg tgctggagag gatgcggaga aataggaaca cttttacact |
| 132721 | gttggtggga ctgtaaacta gttcaaccat tgtggaagtc agtgtggcga ttcctcaggg |
| 132781 | atctagaact agaaatacca tttgacccag ccatcccatt actgggtata tacccaaagg |
| 132841 | actataaatc atgctgctat aaagacacat gcacacgtat gtttattgcg gcactattca |
| 132901 | caatagcaaa gacttggaac caacccaaat gtccaacaat gatagactgg attaagaaaa |
| 132961 | tgtggcacat atacaccatg gaatactatg cagccataaa aaatgatgag ttcatgtcct |
| 133021 | ttgtagggac atggatgaaa ttggaaacca tcattctcag taaactatcg caagaacaaa |
| 133081 | aaaccaaaca ccgcatattc tcactcatag gtgggaattg aacaatgaga tcacatggac |
| 133141 | acaggaaggg gaatatcaca ctctggggac tgtggtgggg tcaggggagg ggggagggat |
| 133201 | agcattggga gatataccta atgctagatg acacgttagt gggtgcagcg caccagcatg |
| 133261 | gcacatgtat acatatgtaa ctaacctgca caatgtgcac atgtacccta aaacttagag |
| 133321 | tataacaaaa aaataaaaaa ataaaaaata aaaaataaat gcatgaatac ccaaaaaaaa |
| 133381 | aaaaaaaaaa aaaaaaaaga aggcatttca gcatgcaaac aagtaatact tttaggaatt |
| 133441 | acactgactt cattgtaatg cataggaaaa ataaataatt cacaaataaa aaacaaacaa |
| 133501 | acaaaaaaaa agtttcagtg gggtcaaagg atttttagag ttagggcagt cgaggcagtg |
| 133561 | aactagggtg aaaaagggag gaagtgatga acaacaggta gagtaactga aaatgagatt |
| 133621 | atgggaggta tacaaatgta tggaatgaca aagtcagggg tatgacaatg tgagtgatta |
| 133681 | gctgaagttg ggcagaggtc taaggaaagg aagtaaagta atttagtggt caggatattg |
| 133741 | gaaggaatct agataaatat taaaagcaag aatttctata gtagtatctc gaatagtttc |
| 133801 | tggcacattg taggcactct ataaatattt gttcaataaa tgaataaacg atattaagta |
| 133861 | cataacaatt catcttgaaa acacttcttc catttcctca actcctaaac attcatttat |
| 133921 | tcattcttca tcacacaaaa atctgaatct ggcttctgcc ccctaccctc cttatttact |
| 133981 | ttctcaaata gtatacattt agtcattaaa caaacataca tacatgcata ttttcaagat |
| 134041 | tatcaagcaa ttcctgtttc aacttttctt aaaatgttga ctctgtattc gtatacagtt |
| 134101 | tataacatcc tgaattcctg aaattgattc aaacagaaac tatgtacagt tttccaattt |
| 134161 | aaccacagta gcatgcttat acatcactac ttggagtttt ctctcatttt tattgtccaa |
| 134221 | caagaactga aacttatgtt gccgtttgtt aaatgctata tctaaattcc atttggatat |
| 134281 | ccaatgaaca caggttctat tttttatcaa gtatacataa gcagtaatca ttcatgcaag |
| 134341 | tgattccact actctcatag tttcttgtaa gttaatccca gtgaaataag atgacaatag |
| 134401 | tggaagaaag gggaattggg aaaatccata aaattcactt ctatcaataa aagaaatcta |
| 134461 | ctttgtttta tatatttgat acatcagcca cttattgaac atgttcgtat cttaaaaatt |
| 134521 | gtttcccttt tattgaaatc ctagtggata atggttttgt agtcataaat tgggaaaaat |
| 134581 | ggtataacta aagaaatatg cagaatttta gaatgggagt aggcattgaa tattatagac |
| 134641 | tcttacggaa tattctggca gtggagcaaa cagtagaaca atcaggtgag attattcatt |
| 134701 | gaagatgtca tgctaaagtt taaagacatg gcttgggcta ggcatgaggt tttcttatgc |
| 134761 | ccatccatct atgcatccat ccttaaatct atccatgcat ccacctatgc attaattcat |
| 134821 | ccatccaccc atcaacatat attgaaaacc catagtgagc catgtgtagt attagacctg |
| 134881 | aggttacaag gatgaataaa atacagtctt cctctcaata attattatat tttgaagtcc |
| 134941 | cacctaagag ttgggttgga gtaattacaa tatagaaaac attttgaggt cttaaataaa |
| 135001 | tgtatataca tatataattg taaattatat ctatacacat gtatatataa ttgttcataa |
| 135061 | taatcacaaa gtagaaatct tttgtatagt attttcttta tcagagtgga aaatttcatt |
| 135121 | aaatcaattt agttcaaaac tttatttctc agagaaatat tttgtcattt ttttccagta |
| 135181 | taagaaatta tttttaaatc aactcaacca gcataacaat ggtaaagcca gttgatgaga |
| 135241 | taggtaagtc cctgttaact aaagattatc caaaaaggat cccacagaag aaacttctaa |
| 135301 | gtattatcca gttggtattt ctttattttt tccctttagt ttacagaaat gtgctaattg |
| 135361 | aggtccttgg aattaaaaat taatgccaaa ataaataagc caagatagca gtattcctca |
| 135421 | tttctcctct ttttcaaaca gatactttta acttttgcct gaaaaaaccc aatagatcta |
| 135481 | ataatctatt tttaagatga tgtgaaatta tgcccatctt atctaatatt ccaagttctc |
| 135541 | cagtttccct ctcaaatata tcaagacttt aagatttgaa aatgatcttc tggctttaaa |
| 135601 | tctaagactt tatttatatt aacttttttg gtagcatgaa cattgtaaaa cctaaatttc |
| 135661 | aaaatgtttt actgcagcaa cttaaatact tcatctaaga cacttgaatt gtatttgtta |
| 135721 | ttgtttccaa acttgataca tctttcttat taacatcctt catagaagta aacagaaaca |
| 135781 | gacagcaatt acattacata ctcagagcaa gcatccattc ataaattaac tcaccaaaca |
| 135841 | ttatgaggca tctgctctgc cagacacgtg gtaggtgtgg ggaattttta aagaagaata |
| 135901 | aggttcaatc tctcctttca aagtgctaaa acgttgggga gaaacctcaa atagtcaaat |
| 135961 | gattataaaa taggatggta ggttctctgt gaaaggatga cagtaggaaa atctaacagt |
| 136021 | tactggaagg atcaggagct gaggcaccct agggagagtg ataaactgaa tcttaaaggc |
| 136081 | aaagaggaga tgtttaggca gagaaatagg gtcagggttc aaccatggtg ttgtcaacaa |
| 136141 | aaaaccaggg cctccagatt tcagagagaa gagccactcc agagaactga tacaccatgt |
| 136201 | acttatatgc accatgaact tgatgcacca tgtacgatgt acttatgaca agtgcccagg |
| 136261 | acaacagcgc tctgctggaa gtcctcaaaa actttagcca agggacagaa accccactac |
| 136321 | ccagagtgca tttcctagct gagactcctt ttgtgttgtg attaaaagga tacaaatatt |
| 136381 | aaaaaacaaa catacgacaa caacaacaaa aaacagggct tcatatgttc tgacatggtc |
| 136441 | gtttgctttt ggagtgctct ttccattgtt tcattgtaga aactgagact aacagggatg |
| 136501 | gtcttttggt tccaggagtg tgtgctgtcg ggaagccaat gtgcctgctc tttgaataca |
| 136561 | tggcctatgg tgacctcaat gagttcctcc gcagcatgtc ccctcacacc gtgtgcagcc |
| 136621 | tcagtcacag tgacttgtct atgagggctc aggtctccag ccctgggccc ccacccctct |
| 136681 | cctgtgctga gcagctttgc attgccaggc aggtggcagc tggcatggct tacctctcag |
| 136741 | aacgtaagtt tgttcaccga gatttagcca ccaggaactg cctggtgggc gagaacatgg |
| 136801 | tggtgaaaat tgccgacttt ggcctctcca ggaacatcta ctcagcagac tactacaaag |
| 136861 | ctaatgaaaa cgacgctatc cctatccgtt ggatgccacc agagtccatt ttttataacc |
| 136921 | gctacactac agagtctgat gtgtgggcct atggcgtggt cctctgggag atcttctcct |
| 136981 | atggcctgca gccctactat gggatggccc atgaggaggt catttactac gtgcgagatg |
| 137041 | gcaacatcct ctcctgccct gagaactgcc ccgtggagct gtacaatctc atgcgtctat |
| 137101 | gttggagcaa gctgcctgca gacagaccca gtttcaccag tattcaccga attctggaac |
| 137161 | gcatgtgtga gagggcagag ggaactgtga gtgtctaagg ttgaagacgt tcaaataaaa |
| 137221 | tgctgcagtt tcctctcaga ctctgtgagc caggggaatc ctacaccaga ggcccaacaa |
| 137281 | gacccacata ggaaagagta agagtaaaca tgagtagtgt gttgtttgct tcccagggag |
| 137341 | agcaaagaca gtgcaaaacc catgtggtag acggacccat tgaaagccag tgattggaaa |
| 137401 | cacaggctag gaaatgtgtc agataatgga gacaactatt cttcttcatg aaggtttgta |
| 137461 | atacacactg cacagggaag aatgtcatcc tgttcagttt ccaaagtagc tggtcacaga |
| 137521 | gtaaagctct gctgtattaa attttaatat gaaatgggat tcagagcttc attttttaat |
| 137581 | tgagttaatt cctgtccatt gtgaaagtag cttaatcgtc atgtaagaca cttaggtgaa |
| 137641 | gtacagaaaa ctaaaaagaa ggaaaagcca ctcattctgt ctccccccaa agacgagtct |
| 137701 | tagtgtttta ttaaatcttt cttgcattta atgcataatt ttatatttgt ttgtattctg |
| 137761 | cagagttgtg gttacatttt attgatagag ggtttctaag gaatgaaaca gcaaatcagc |
| 137821 | aagcctgtaa ctctgagttc ccttttctgt cctgtgcccc tccttttttc ctttctgtgc |
| 137881 | atttgtcatg aattaagtct gcttatttta ttcctccagt tttcttgaaa caagtttttg |
| 137941 | tatcttactg atttaccttt tcctttaaaa attgtattgg tatcttttcc attgctttaa |
| 138001 | tctttatttc ttcttctttc caggacttct gtcctttttt agttattaag ctattttttt |
| 138061 | cttttatttc attcatgtaa taaacactct ggcagaaata ataacatttt aaataccatt |
| 138121 | gtagattgat atgtatatgc cttttgcaaa gtattactat ttttcctgca tttgcctttg |
| 138181 | aagccacaca tttcaacatt ctcacctatt aatattatta ctagagtcta tttaaatctc |
| 138241 | ttaaaccata atttataata tgagatttca caagatacac ttgtggctct gagttatttc |
| 138301 | aaaactgata tttctgtgca ttctgacatt ttattccttt tagaaacaaa ttcagttggc |
| 138361 | tgtagaatta atatcgttct gcttaacaac tttgtgcatg acatattaac tgattttgtt |
| 138421 | gtatttcatg atttaaaaaa aaaagcagca gctctggaac tcagattcct gggggttcag |
| 138481 | attttttctc agtggatcct cacacaatac atctttatct aagtccatac atgatctgag |
| 138541 | tttatttctg atatagcagg aacccaaatt ctcctgaaaa cgaacaggga agagaaaata |
| 138601 | acagtctact ctataccagc ttatcttaat tcagactaat tggtagttag ggcattggag |
| 138661 | gaatggtgtg aattcttgaa agtagatttt cgctgtctgc tggaaaactc tgcctgaaat |
| 138721 | ccccaaatct gcctcatgta acagcttgac atctttggtg ggaaacctgc catttcttat |
| 138781 | tcatagtgtc tccaggcaac caagacaaaa atgtagaaat tttcctagac tttgccctct |
| 138841 | ctttcatcat cagttttcta ttagttttac ttcctaaaat cctcctgctg acatcctctt |
| 138901 | cccttcaacc caagagccaa tgactcaacc tctaccgttg cccaccctgt ctattagctg |
| 138961 | gccttcctct catttcagac ctgaacagat tctgtaggat acaacgacag taaggcggtc |
| 139021 | cttagtcaca gttctggttc ataaatagcg aggtcagcat ttaggactga gacctaaaga |
| 139081 | agtgcttcag ataaaaatca tgagtctaag tccaaaattg atactgtaat ccaaatgact |
| 139141 | gaataaagaa taaagttgca gcgtgtgtct aaagcagaaa gtcaagatgc caacaaagca |
| 139201 | gaaaagttgg agagggccac cagcaagtct agaacttgag catagatatc catggcttat |
| 139261 | tttagatggt tcatgtatgc tctgcaaaat tcaaagacag cttcgggctt tctccgctga |
| 139321 | taaagttttg ttgtaaaaga cagacataga agtcaggctg acagaaaata gagtaacagt |
| 139381 | aatcataaaa attataatat cagctagcat ttattgaaat cttaccatgt gtattctgag |
| 139441 | gcctttaaat atatgaactt aaacctcaca acatccctgt aatgttggta ctatcattat |
| 139501 | atacatttta gttttgcttt gttttgtttt gtttttgaga cggagtctcg ctctgtcgcc |
| 139561 | caggctggag tgcagtggca cgatcttggc tcactgcaag ctcagcctcc aacagtcaca |
| 139621 | gagagattgt gcaactggcc cgagtgcaca cagcaacaga gcctactttt tgaccaaaag |
| 139681 | aggccgaatt ctcaaccact atactgtaga gcctctgtac atgatagaag caataactga |
| 139741 | acctcagtaa ctctgtggaa gtgatttgta atttgcatgt cgtttcaagc acactctata |
| 139801 | tctatatttt attctattaa cttcctaagt ctttttaatg aatagcaaaa aaagtatatt |
| 139861 | tcagcattat caatttaagt taatcataac atctaatagg tgacatgttt attttttcat |
| 139921 | gagttctaat tacattttca agttaagatc actgacttat tccctaagtg ctcgatctaa |
| 139981 | tgttctgaat cacaggagta tagatgcatt attcaccaat attgtttact tttttaaata |
| 140041 | agattttttt tcatggtaga atcatcatat acattcaaaa aggtagacaa aagagagaca |
| 140101 | ctcacagtgt cactgttacc aagtgccagg acagatatca gtaaggcacc agcagtcatt |
| 140161 | tttagtgatc aaataccaaa catggtaaga taccagagta tttttcacat gacacccaaa |
| 140221 | aacatattct ctgctatcgt ggtagagcag aaaaagaaca ctttcttttc attttacagt |
| 140281 | atattacgaa cctgttccac gtcattaaat attcttctaa aaacccaatt ttggaataat |
| 140341 | attctatttt attcatatac tgtaatttag ctaatcaatc ttgtattttt gaacatttag |
| 140401 | gttttttcca atttttatca ttataaaaaa tgctgtaatt aacatcctta tttgttcatg |
| 140461 | tccacatttc tttttatgaa aattcagaga aaggaaatgt ctggaccaaa tggcatgtac |
| 140521 | aatattaaca cttctgttat gtggtttcat attctgcaga aatattactg ccacttctac |
| 140581 | attgtacaag agatctcata gccctaaaca tttgctttca tagataattt tctgtttttt |
| 140641 | agaaatcttt tgtctctttg gttatagaaa aagagaacct ctttttaatt ttatttttta |
| 140701 | aattactagt caggttgaat gtttcttgtt cctaggtcat taacagtttt tattttggca |
| 140761 | attgtctaca gcccattttt ctgctgtgtt cattttctaa tcgaattatg ggatttatct |
| 140821 | tgtaaaaagc ctattaagcc tttttcatac tttacacaag agatcacatc tacttacttt |
| 140881 | accttaaaat aacatttact tattttctta taaaagtaat atattatgtt tgaaaagaaa |
| 140941 | aataatatgc aataatatac aataatatgt ataatcataa tagacaataa taaaaatgaa |
| 141001 | aaaacaccaa ccagaggtaa ttaacattaa catcataata ttcttccagg ctttttcaga |
| 141061 | tagacaggta ggtaagtagg ttggtaggta aatagataga tagatgatac atagagagag |
| 141121 | atagatagat aatttaaaca aaaataagat catactgcta cattacttca agccttgctt |
| 141181 | ttatcactta atatatctta aacatcttct acatgaacaa gtattcatgt gcaacattaa |
| 141241 | ttttgatggt tgaatagtat tatacagatt tatcgtaatt gacataacta ttctactgtt |
| 141301 | agacatttgg tttatttcta tttttccatt ataaaatgta atgtttggat catcctgttg |
| 141361 | ctaaattttg taagcattta tcttaagtga tttctttagc tataactcct agaagtgaaa |
| 141421 | tttctagatt aaaggataaa tgcactttta atacttttta aatatattaa aaaattacct |
| 141481 | tcctgaatta ttttattaac atgtacctct cctagtcaga taagacagcc catttatttc |
| 141541 | agtctcacca atactgagta tagcatttta atcattgcca atataatagc taatagctta |
| 141601 | aagttatttc atagcttata tttacacttc tttaaatact aataaaattg aacattgttt |
| 141661 | aatgttttga ggatacttga ttggtgattt gcctcctact attatttttc tactgggtat |
| 141721 | atctttttct tgttgctttg taagagcact ttatatatta aggagttaaa ttttgtgata |
| 141781 | tatgtttcaa ctacttttat catgttaaaa atatagactt ctatttgtct tttattgtag |
| 141841 | gttttatcaa tcagaaaaga atgttgactt ttccgtccat tcagatgatt gtggcttttt |
| 141901 | tccctaatgg atttgttaat atgttcaatt gtatgaattt atttcctaat gtagcgatat |
| 141961 | ccttgtcttc taggaataaa ctttttgggc gaatcatatc aatcttctac ttaactacca |
| 142021 | aattaattta catatatttg catcaatatt aagttcataa aattaaaagt ttatcatttt |
| 142081 | catctctgtt agagttttgt attggcatta gcatttgtat tagcattgta ttaggaaaaa |
| 142141 | ctcaggagtt tttctctata atcttggtta gtttattttt aaataattgt tatttatgaa |
| 142201 | agtaatacat aaaaatgttg tttaaaaaaa ctcacaagag ggtaaaaatt aaaaaaataa |
| 142261 | taaaagttcc cttcttcctt ttcatctcta gtcccagcct aagtgctaac cacttcaccc |
| 142321 | atttatagtg ttagttcttc tggtgattac cagtgcaatt ctaacaaata ggtttgcatt |
| 142381 | tctctttctg aattgatcaa ttttttaaaa atatgcagca ctctgactca aactcccttt |
| 142441 | ctttttcctg agatatgtta acttctaatg ttatattgtt tatcacaact ttcacaaata |
| 142501 | tttctaaata aaggttacct actgattctg gattgtgcta cataaaatta gagcttccaa |
| 142561 | atttccctcc atcacttctc cctgctctct actctcacaa tcagtcaact atacacctat |
| 142621 | ttctacgctg tcaactatat gttcatttct atgaatctgt aatatttgca ttttgttctg |
| 142681 | gaacaaatta agatttaggt gctttgacta gattgattct aaacatcaaa aaacaaaaat |
| 142741 | aaaatatctg caattatgtg atcatgtatt tatgcattgc caaataatgg tattccatta |
| 142801 | ttttaatttg ataagataaa tttaatatac attaattctt atttcctcta ttttatggaa |
| 142861 | gaaatatttt taaaaatcac aaaactttta cattcgcttc tcttcctgat atttattttt |
| 142921 | cctttttctt ctgcctctct tctttcgtgc ccttttttcc gaaaattatt ttgatgattt |
| 142981 | tttttattca ccatacataa ttacaaatga agagttgctc tggttcgtat tggtagtaga |
| 143041 | agtggggtcc ctttgctgtt ttgttggtat attcacccag tgtgacctct tttccgtggg |
| 143101 | atggctacct ctgggctctg attatgtggc tcatagggaa gtataggcag cctgagtcag |
| 143161 | aatccccaca gttccaaagt gatggaagac tgtactctga gatacaatat tttgacgtat |
| 143221 | ttcactccca gggagagcta cttgctattc tttcactgct tccattaccc aaaagagtat |
| 143281 | gatattctcc ccattttctg tattgcctca cacctaggac ccttcaaacc cacaccaacg |
| 143341 | acctctcagg caaattgatc tattcagata gcagtttaca gggatagaca tgaggcctct |
| 143401 | tggcccacct gctctataca caatcttcta gtcgaccatc ctgattgaca acccacagcc |
| 143461 | cacccctgtc tctgtatttg ctgactcaga gcatggagac tttatacatg tgaaatcaca |
| 143521 | tctcccctcc cctcacgctt ggcttgtgtg tattgcaaaa gctgcaattt agtttttcct |
| 143581 | gtgccttgtc ttacccatgg ataagaacac taagatactt tccgcttttt gaatatttgt |
| 143641 | caaagtgatg gcacttggat tggtttcagc atgtacatag attgattctt attcatatac |
| 143701 | atttactgtc atattgggga aatcttgagg ggaaaagaaa agcctcttac taccatgttc |
| 143761 | atcaagagtc ctctctggaa tagttgaaat agtttaggag ttatcggttc actccttcgt |
| 143821 | cttgtttgcc tccagatttc ttttttcctt cttttcctgt ttcttaatgt tggaacagcc |
| 143881 | cacacctcaa tccttggatg acatctccct tttatctgta ttcatcccta gtgatctctt |
| 143941 | ctggttgcaa ggctataaat gccatctgta tgctgagatt tttttatttt tagttcaaac |
| 144001 | ctctctcccc aactttacac tcacatttct aactgtgtgt tatagatgat agctttagat |
| 144061 | tctcctcctc aggactaggc tttctgcttg atctaggagc aaagctgaaa actgctatgg |
| 144121 | agcttggtgt tgccttgata ccgaggagga gtctcagagc ctctccagtg caaatacaca |
| 144181 | cttgcagtgt cctagtgctg ctgacttacg tactcattct aaagagggta gattgtatcc |
| 144241 | attttaatag ctctcaatac ccttgcccat tcccattaca gctggcagaa attccccagc |
| 144301 | tgcaaatggg ttgcatttat ctgttaccca ttcctgattt gcagcacgat tgactgtttc |
| 144361 | cttttctgcg tggtctatgt gtcactccat gagaatctgg gagaggagaa ctcgatatgg |
| 144421 | tgctcattcc atcattttaa ccatagtgtt ctttattgtg aagaggtatc ccggttagag |
| 144481 | tggagtcaaa gctttatagc aagtgtattt caggtaacta ttcttttact tgtttcagat |
| 144541 | ctatttcagt agcacagcca ccaagataga acacttataa gcccttattt tggtatagat |
| 144601 | caccagagaa accactcacc actcgcactt ggccttcagt actgagtcat tacatattgg |
| 144661 | aaggtcagaa actacatcaa caggaaagac ccataggtgt gggggtctga tatatttgcc |
| 144721 | agtacatttc ttatcattac aaacaaagat ttatgtgtgt ctagatgt |

hu6-75; SEQ ID NO: 78
G*C*T *A*G*G *G*T*G *G*T*C *T*T*T *T*A*G *A*A*A *T*G*C *A
*represents a phosphorothioate linkage, and each sugar of the
oligonucleotides is a 2′ -MOE modified sugar.
hu6-69; SEQ ID NO: 79
G*G*T *C*A*A *G*C*T *A*G*G *G*T*G *G*T*C *T*T*T *T*A*G *A
hu6-58; SEQ ID NO: 80
C*T*G *C*A*G *G*A*A *A*T*G *G*T*C *A*A*G *C*T*A *G*G*G *T
hu6-39; SEQ ID NO: 81
G*A*A *G*T*G *G*T*G *A*G*T *G*A*C *G*C*T *C*C*T *G*C*A *G
hu6-27; SEQ ID NO: 82
G*T*T *A*G*G *A*A*G *A*C*A *G*A*A *G*T*G *G*T*G *A*G*T *G
hu6-12; SEQ ID NO: 83
A*T*C *C*T*G *G*C*A *A*A*A *A*C*T *G*T*T *A*G*G *A*A*G *A
hu610; SEQ ID NO: 84
G*T*G *G*G*A *T*T*C *A*G*G *A*G*C *C*C*G *C*A*G *G*A*T *C
hu619; SEQ ID NO: 85
G*G*T *G*A*C *A*T*T *G*T*G *G*G*A *T*T*C *A*G*G *A*G*C *C
hu628; SEQ ID NO: 86
G*G*A *G*C*C *A*A*A *G*G*T *G*A*C *A*T*T *G*T*G *G*G*A *T
hu637; SEQ ID NO: 87
G*G*T *C*A*C *A*A*A *G*G*A *G*C*C *A*A*A *G*G*T *G*A*C *A
hu646; SEQ ID NO: 88
A*C*A *G*T*G *C*A*G *G*G*T *C*A*C *A*A*A *G*G*A *G*C*C *A
hu656; SEQ ID NO: 89
C*T*G *T*T*G *C*T*G *T*A*C *A*G*T *G*C*A *G*G*G *T*C*A *C
hu669; SEQ ID NO: 90
G*G*G *A*C*A *G*G*A *A*T*G *C*C*T *G*T*T *G*C*T *G*T*A *C
hu681; SEQ ID NO: 91
C*A*G *G*T*G *A*T*G *G*T*G *G*G*G *A*C*A *G*G*A *A*T*G *C
hu693; SEQ ID NO: 92
C*C*G *T*T*T *T*C*A *A*T*C *C*A*G *G*T*G *A*T*G *G*T*G *G
hu6110; SEQ ID NO: 93
T*G*A *C*A*C *T*C*A *C*A*G *C*A*T *T*T*C *C*G*T *T*T*T *C
hu6125; SEQ ID NO: 94
A*A*G *T*C*C *C*C*A *C*A*C *A*C*A *T*G*A *C*A*C *T*C*A *C
hu6139; SEQ ID NO: 95
G*G*T *C*T*T *C*C*C *C*A*G *A*C*A *A*G*T *C*C*C *C*A*C *A
hu7-75; SEQ ID NO: 96
A*C*T *A*T*G *T*C*A *G*T*A *G*A*T *T*T*G *A*A*G *G*G*A *A
hu7-66; SEQ ID NO: 97
T*C*C *C*A*C *T*A*T *A*C*T *A*T*G *T*C*A *G*T*A *G*A*T *T
hu7-53; SEQ ID NO: 98
T*C*A *G*T*C *A*A*G *G*A*T *T*T*C *C*C*A *C*T*A *T*A*C *T
hu7-45; SEQ ID NO: 99
A*A*A *A*G*A *A*C*T *C*A*G *T*C*A *A*G*G *A*T*T *T*C*C *C
hu7-33; SEQ ID NO: 100
G*T*A *A*A*G *G*A*A *A*A*T *A*A*A *A*G*A *A*C*T *C*A*G *T
hu7-22; SEQ ID NO: 101
A*A*C *C*T*G *A*C*A *G*A*G *T*A*A *A*G*G *A*A*A *A*T*A *A
hu7-10; SEQ ID NO: 102
G*G*A *C*C*C *A*G*A *A*G*A *A*A*C *C*T*G *A*C*A *G*A*G *T
hu73; SEQ ID NO: 103
C*A*C *T*C*T *C*T*T *G*A*A *T*G*G *A*C*C *C*A*G *A*A*G *A
hu717; SEQ ID NO: 104
C*A*C *T*C*G *G*T*C *T*T*T *C*A*C *A*C*T *C*T*C *T*T*G *A
hu730; SEQ ID NO: 105
G*T*C *T*T*G *A*G*T *C*A*A *T*C*A *C*T*C *G*G*T *C*T*T *T
hu744; SEQ ID NO: 106
G*A*T *A*A*A *C*A*G *C*T*G *C*A*G *T*C*T *T*G*A *G*T*C *A
hu758; SEQ ID NO: 107
A*G*T *C*C*T *G*G*C *T*T*G *G*T*G *A*T*A *A*A*C *A*G*C *T
hu766; SEQ ID NO: 108
A*T*G *T*G*T *A*G*A *G*T*C *C*T*G *G*C*T *T*G*G *T*G*A *T
hu776; SEQ ID NO: 109
G*T*A *G*C*T *A*T*G *C*A*T *G*T*G *T*A*G *A*G*T *C*C*T *G
hu785; SEQ ID NO: 110
T*G*C *T*T*A *T*T*G *G*T*A *G*C*T *A*T*G *C*A*T *G*T*G *T
hu796; SEQ ID NO: 111
A*C*T *T*C*T *C*C*C *C*A*T *G*C*T *T*A*T *T*G*G *T*A*G *C
hu7111; SEQ ID NO: 112
C*C*T *T*G*G *C*A*G *T*A*C *T*G*A *A*C*T *T*C*T *C*C*C *C
hu7136; SEQ ID NO: 113
C*C*T *G*C*T *A*T*G *C*T*G *A*T*G *G*T*G *G*C*T *G*C*A *G
hu7146; SEQ ID NO: 114
G*G*G *C*A*T *C*C*T *A*C*C *T*G*C *T*A*T *G*C*T *G*A*T *G
hu7158; SEQ ID NO: 115
G*C*A *A*A*T *G*T*G *A*A*G *G*G*G *C*A*T *C*C*T *A*C*C *T
MuSK HUMAN_Ig3_Domain (SEQ ID NO: 116)
ARILRAPESHNVTFGSFVTLHCTATGIPVPTITWIENGNAVSSGSIQESVKDRVIDSRLQLFITKPGLYTCIAT
NKHGEKFSTAKAAATIS
MuSK MOUSE Ig3 Domain (SEQ ID NO: 117)
ARILRAPESHNVTFGSFVTLRCTAIGIPVPTISWIENGNAVSSGSIQESVKDRVIDSRLQLFITKPGLYTCIAT
NKHGEKEFTAKAAATVS
MuSK 34 (spanning the exon/exon junction 3-4) Forward
CCTGCAAGTGAAGATGAAACCTAAA (SEQ ID NO: 118)
MuSK 34 (spanning the exon/exon junction 3-4) Reverse
ATGAATCCTCAAGCTCCCAGA (SEQ ID NO: 119)
MuSK 67 (spanning the exon/exon junction 6-7) Forward
GGCTCCTGAATCCCACAATG (SEQ ID NO: 120)
MuSK 67 (spanning the exon/exon junction 6-7) Reverse
GAATGGACCCAGAAGAAACAGCA (SEQ ID NO: 121)
GAPDH (HKG) Forward
CCTCAACGACCACTTTGTCA (SEQ ID NO: 122)
GAPDH (HKG) Reverse
TTACTCCTTGGAGGCCATGT (SEQ ID NO: 123)
YWHAZ (HKG) Forward
CGAAGCTGAAGCAGGAGAAG (SEQ ID NO: 124)
YWHAZ (HKG) Reverse
TTTGTGGGACAGCATGGATG (SEQ ID NO: 125)
Region 1 - MuSK Exon 7
ACTCTGTCAGGTTTCTTCTGGGTCCATTCAAGAGAGTGTGAAAGACCGAGTGATTGACTCA
AGAC (SEQ ID NO: 126)
Oligonucleotide Bld51 (SEQ ID NO: 127)
TTGAATGGACCCAGAAGAAA
Oligonucleotide Bld52 (SEQ ID NO: 128)
CTTGAATGGACCCAGAAGAA
Oligonucleotide Bld53 (SEQ ID NO: 129)
TCTTGAATGGACCCAGAAGA
Oligonucleotide Bld54 (SEQ ID NO: 130)
CTCTTGAATGGACCCAGAAG
Oligonucleotide Bld55 (SEQ ID NO: 131)
TCTCTTGAATGGACCCAGAA
Oligonucleotide Bld56 (SEQ ID NO: 132)
CTCTCTTGAATGGACCCAGA
Oligonucleotide Bld57 (SEQ ID NO: 133)
ACTCTCTTGAATGGACCCAG
Oligonucleotide Bld58 (SEQ ID NO: 134)
CACTCTCTTGAATGGACCCA
Oligonucleotide Bld59 (SEQ ID NO: 135)
ACACTCTCTTGAATGGACCC
Oligonucleotide Bld60 (SEQ ID NO: 136)
CACACTCTCTTGAATGGACC
Oligonucleotide Bld61 (SEQ ID NO: 137)
TCACACTCTCTTGAATGGAC
Oligonucleotide Bld62 (SEQ ID NO: 138)
TTCACACTCTCTTGAATGGA
Oligonucleotide Bld63 (SEQ ID NO: 139)
TTTCACACTCTCTTGAATGG
Oligonucleotide Bld64 (SEQ ID NO: 140)
CTTTCACACTCTCTTGAATG
Oligonucleotide Bld65 (SEQ ID NO: 141)
TCTTTCACACTCTCTTGAAT
Oligonucleotide Bld66 (SEQ ID NO: 142)
GTCTTTCACACTCTCTTGAA
Bld51; SEQ ID NO: 143
T*T*G*A*A*T*G*G*A*C*C*C*A*G*A*A*G*A*A*A
Bld52; SEQ ID NO: 144
C*T*T*G*A*A*T*G*G*A*C*C*C*A*G*A*A*G*A*A
Bld53; SEQ ID NO: 145
T*C*T*T*G*A*A*T*G*G*A*C*C*C*A*G*A*A*G*A
Bld54; SEQ ID NO: 146
C*T*C*T*T*G*A*A*T*G*G*A*C*C*C*A*G*A*A*G
Bld55; SEQ ID NO: 147
T*C*T*C*T*T*G*A*A*T*G*G*A*C*C*C*A*G*A*A
Bld56; SEQ ID NO: 148
C*T*C*T*C*T*T*G*A*A*T*G*G*A*C*C*C*A*G*A
Bld57; SEQ ID NO: 149
A*C*T*C*T*C*T*T*G*A*A*T*G*G*A*C*C*C*A*G
Bld58; SEQ ID NO: 150
C*A*C*T*C*T*C*T*T*G*A*A*T*G*G*A*C*C*C*A
Bld59; SEQ ID NO: 151
A*C*A*C*T*C*T*C*T*T*G*A*A*T*G*G*A*C*C*C
Bld60; SEQ ID NO: 152
C*A*C*A*C*T*C*T*C*T*T*G*A*A*T*G*G*A*C*C
Bld61; SEQ ID NO: 153
T*C*A*C*A*C*T*C*T*C*T*T*G*A*A*T*G*G*A*C
Bld62; SEQ ID NO: 154
T*T*C*A*C*A*C*T*C*T*C*T*T*G*A*A*T*G*G*A
Bld63; SEQ ID NO: 155
T*T*T*C*A*C*A*C*T*C*T*C*T*T*G*A*A*T*G*G
Bld64; SEQ ID NO: 156
C*T*T*T*C*A*C*A*C*T*C*T*C&T*T*G*A*A*T*G
Bld65; SEQ ID NO: 157
T*C*T*T*T*C*A*C*A*C*T*C*T*C*T*T*G*A*A*T
Bld66; SEQ ID NO: 158
G*T*C*T*T*T*C*A*C*A*C*T*C*T*C*T*T*G*A*A
Oligonucleotide Bld25-1 (SEQ ID NO: 159)
CCAGAAGAAACCTGACAGAGTAAAG
Oligonucleotide Bld25-2 (SEQ ID NO: 160)
ACCCAGAAGAAACCTGACAGAGTAA
Oligonucleotide Bld25-3 (SEQ ID NO: 161)
ATGGACCCAGAAGAAACCTGACAGA
Oligonucleotide Bld25-4 (SEQ ID NO: 162)
GAATGGACCCAGAAGAAACCTGACA
Oligonucleotide Bld25-5 (SEQ ID NO: 163)
CTTGAATGGACCCAGAAGAAACCTG
Oligonucleotide Bld26-1 (SEQ ID NO: 164)
CTCTTGAATGGACCCAGAAGAAACC
Oligonucleotide Bld26-2 (SEQ ID NO: 165)
CTCTCTTGAATGGACCCAGAAGAAA
Oligonucleotide Bld26-3 (SEQ ID NO: 166)
CACACTCTCTTGAATGGACCCAGAA
Oligonucleotide Bld26-4 (SEQ ID NO: 167)
TTCACACTCTCTTGAATGGACCCAG
Bld25-1; SEQ ID NO: 168
C*C*A*G*A*A*G*A*A*A*C*C*T*G*A*C*A*G*A*G*T*A*A*A*G*
Bld25-2; SEQ ID NO: 169
A*C*C*C*A*G*A*A*G*A*A*A*C*C*T*G*A*C*A*G*A*G*T*A*A*
Bld25-3; SEQ ID NO: 170
A*T*G*G*A*C*C*C*A*G*A*A*G*A*A*A*C*C*T*G*A*C*A*G*A*
Bld25-4; SEQ ID NO: 171
G*A*A*T*G*G*A*C*C*C*A*G*A*A*G*A*A*A*C*C*T*G*A*C*A*
Bld25-5; SEQ ID NO: 172
C*T*T*G*A*A*T*G*G*A*C*C*C*A*G*A*A*G*A*A*A*C*C*T*G*
Bld26-1; SEQ ID NO: 173
C*T*C*T*T*G*A*A*T*G*G*A*C*C*C*A*G*A*A*G*A*A*A*C*C*
Bld26-2; SEQ ID NO: 174
C*T*C*T*C*T*T*G*A*A*T*G*G*A*C*C*C*A*G*A*A*G*A*A*A*
Bld26-3; SEQ ID NO: 175
C*A*C*A*C*T*C*T*C*T*T*G*A*A*T*G*G*A*C*C*C*A*G*A*A*
Bld26-4; SEQ ID NO: 176
T*T*C*A*C*A*C*T*C*T*C*T*T*G*A*A*T*G*G*A*C*C*C*A*G*
Oligonucleotide Bld25-A (SEQ ID NO: 177)
ACCCAGAAGAAACCTGACAGAGT
Oligonucleotide Bld25-B (SEQ ID NO: 178)
CCAGAAGAAACCTGACAGAGT
Oligonucleotide Bld25-C (SEQ ID NO: 179)
GGACCCAGAAGAAACCTGACAGA
Oligonucleotide Bld25-D (SEQ ID NO: 180)
ACCCAGAAGAAACCTGACAGA
Oligonucleotide Bld25-E (SEQ ID NO: 181)
GGACCCAGAAGAAACCTGACA
Oligonucleotide Bld25-5-A (SEQ ID NO: 182)
GAATGGACCCAGAAGAAACCTGA
Oligonucleotide Bld25-5-B (SEQ ID NO: 183)
ATGGACCCAGAAGAAACCTGA
Oligonucleotide Bld25-5-C (SEQ ID NO: 184)
TTGAATGGACCCAGAAGAAACCT
Oligonucleotide Bld25-5-D (SEQ ID NO: 185)
GAATGGACCCAGAAGAAACCT
Oligonucleotide Bld25-5-E (SEQ ID NO: 186)
TTGAATGGACCCAGAAGAAAC
Oligonucleotide Bld26-2-A (SEQ ID NO: 187)
CTCTTGAATGGACCCAGAAGAAA
Oligonucleotide Bld26-2-B (SEQ ID NO: 188)
CTTGAATGGACCCAGAAGAAA
Oligonucleotide Bld26-2-C (SEQ ID NO: 189)
CTCTCTTGAATGGACCCAGAAGA
Oligonucleotide Bld26-2-D (SEQ ID NO: 190)
CTCTCTTGAATGGACCCAGAA
Oligonucleotide Bld26-B (SEQ ID NO: 191)
CTCTTGAATGGACCCAGAAGA
Oligonucleotide Bld26-C (SEQ ID NO: 192)
CACTCTCTTGAATGGACCCAGAA
Oligonucleotide Bld26-D (SEQ ID NO: 193)
CACTCTCTTGAATGGACCCAG
Bld25-A; SEQ ID NO: 194
A*C*C*C*A*G*A*A*G*A*A*A*C*C*T*G*A*C*A*G*A*G*T
Bld25-B; SEQ ID NO: 195
C*C*A*G*A*A*G*A*A*A*C*C*T*G*A*C*A*G*A*G*T
Bld25-C; SEQ ID NO: 196
G*G*A*C*C*C*A*G*A*A*G*A*A*A*C*C*T*G*A*C*A*G*A
Bld25-D; SEQ ID NO: 197
A*C*C*C*A*G*A*A*G*A*A*A*C*C*T*G*A*C*A*G*A
Bld25-E; SEQ ID NO: 198
G*G*A*C*C*C*A*G*A*A*G*A*A*A*C*C*T*G*A*C*A
Bld25-5-A; SEQ ID NO: 199
G*A*A*T*G*G*A*C*C*C*A*G*A*A*G*A*A*A*C*C*T*G*A
Bld25-5-B; SEQ ID NO: 200
A*T*G*G*A*C*C*C*A*G*A*A*G*A*A*A*C*C*T*G*A
Bld25-5-C; SEQ ID NO: 201
T*T*G*A*A*T*G*G*A*C*C*C*A*G*A*A*G*A*A*A*C*C*T
Bld25-5-D; SEQ ID NO: 202
G*A*A*T*G*G*A*C*C*C*A*G*A*A*G*A*A*A*C*C*T
Bld25-5-E; SEQ ID NO: 203
T*T*G*A*A*T*G*G*A*C*C*C*A*G*A*A*G*A*A*A*C
Bld26-2-A; SEQ ID NO: 204
C*T*C*T*T*G*A*A*T*G*G*A*C*C*C*A*G*A*A*G*A*A*A
Bld26-2-B; SEQ ID NO: 205
C*T*T*G*A*A*T*G*G*A*C*C*C*A*G*A*A*G*A*A*A
Bld26-2-C; SEQ ID NO: 206
C*T*C*T*C*T*T*G*A*A*T*G*G*A*C*C*C*A*G*A*A*G*A
Bld26-2-D; SEQ ID NO: 207
C*T*C*T*C*T*T*G*A*A*T*G*G*A*C*C*C*A*G*A*A
Bld26-B; SEQ ID NO: 208
C*T*C*T*T*G*A*A*T*G*G*A*C*C*C*A*G*A*A*G*A
Bld26-C; SEQ ID NO: 209
C*A*C*T*C*T*C*T*T*G*A*A*T*G*G*A*C*C*C*A*G*A*A
Bld26-D; SEQ ID NO: 210
C*A*C*T*C*T*C*T*T*G*A*A*T*G*G*A*C*C*C*A*G
Region 2 - MuSK Exon 7
GGGGAGAAGTTCAGTACTGCCAAGGCTGCAGCCACCATCAGCATAGCAGGTAGGATGCCCC
TTCACATTTG (SEQ ID NO: 211)
SEQ ID NO: 212
TTTCTTCTGGGTCCATTCAA
SEQ ID NO: 213
TTCTTCTGGGTCCATTCAAG
SEQ ID NO: 214
TCTTCTGGGTCCATTCAAGA
SEQ ID NO: 215
CTTCTGGGTCCATTCAAGAG
SEQ ID NO: 216
TTCTGGGTCCATTCAAGAGA
SEQ ID NO: 217
TCTGGGTCCATTCAAGAGAG
SEQ ID NO: 218
CTGGGTCCATTCAAGAGAGT
SEQ ID NO: 219
TGGGTCCATTCAAGAGAGTG
SEQ ID NO: 220
GGGTCCATTCAAGAGAGTGT
SEQ ID NO: 221
GGTCCATTCAAGAGAGTGTG
SEQ ID NO: 222
GTCCATTCAAGAGAGTGTGA
SEQ ID NO: 223
TCCATTCAAGAGAGTGTGAA
SEQ ID NO: 224
CCATTCAAGAGAGTGTGAAA
SEQ ID NO: 225
CATTCAAGAGAGTGTGAAAG
SEQ ID NO: 226
ATTCAAGAGAGTGTGAAAGA
SEQ ID NO: 227
TTCAAGAGAGTGTGAAAGAC
SEQ ID NO: 228
CTTTACTCTGTCAGGTTTCTTCTGG
SEQ ID NO: 229
TTACTCTGTCAGGTTTCTTCTGGGT
SEQ ID NO: 230
TCTGTCAGGTTTCTTCTGGGTCCAT
SEQ ID NO: 231
TGTCAGGTTTCTTCTGGGTCCATTC
SEQ ID NO: 232
CAGGTTTCTTCTGGGTCCATTCAAG
SEQ ID NO: 233
GGTTTCTTCTGGGTCCATTCAAGAG
SEQ ID NO: 234
TTTCTTCTGGGTCCATTCAAGAGAG
SEQ ID NO: 235
TTCTGGGTCCATTCAAGAGAGTGTG
SEQ ID NO: 236
CTGGGTCCATTCAAGAGAGTGTGAA
SEQ ID NO: 237
ACTCTGTCAGGTTTCTTCTGGGT
SEQ ID NO: 238
ACTCTGTCAGGTTTCTTCTGG
SEQ ID NO: 239
TCTGTCAGGTTTCTTCTGGGTCC
SEQ ID NO: 240
TCTGTCAGGTTTCTTCTGGGT
SEQ ID NO: 241
TGTCAGGTTTCTTCTGGGTCC
SEQ ID NO: 242
TCAGGTTTCTTCTGGGTCCATTC
SEQ ID NO: 243
TCAGGTTTCTTCTGGGTCCAT
SEQ ID NO: 244
AGGTTTCTTCTGGGTCCATTCAA
SEQ ID NO: 245
AGGTTTCTTCTGGGTCCATTC
SEQ ID NO: 246
GTTTCTTCTGGGTCCATTCAA
SEQ ID NO: 247
TTTCTTCTGGGTCCATTCAAGAG
SEQ ID NO: 248
TTTCTTCTGGGTCCATTCAAG
SEQ ID NO: 249
TCTTCTGGGTCCATTCAAGAGAG
SEQ ID NO: 250
TTCTGGGTCCATTCAAGAGAG
SEQ ID NO: 251
TCTTCTGGGTCCATTCAAGAG
SEQ ID NO: 252
TTCTGGGTCCATTCAAGAGAGTG
SEQ ID NO: 253
CTGGGTCCATTCAAGAGAGTG

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An oligonucleotide composition, comprising plurality of oligonucleotides:
the oligonucleotide composition being characterized in that, when it is contacted with a MuSK transcript in a transcript splicing system, relative amounts of transcripts that do not include Ig3 domain-encoding sequences are decreased as compared with such relative amounts observed under reference conditions selected from the group consisting of absence of the composition, presence of a reference composition, and combinations thereof;
wherein the oligonucleotides mediate skipping of exon 6 and/or exon 7 of a MuSK gene and MuSK splicing is altered in that level of MuSK transcripts including exons 6 and 7 is decreased or level of MuSK protein forms including sequences encoded by exons 6 and 7 is decreased, or both; and
wherein the base sequence of the oligonucleotides comprise a sequence having no more than 5 mismatches from a 18-25 base long portion of the MuSK gene or its complement.

2. (canceled)

3. The composition of claim 1, wherein the exon skipping lowers levels of mRNAs encoding MuSK protein form that participate in BMP signaling compared with levels observed absent the exon skipping.

4-15. (canceled)

16. The composition of claim 1, wherein MuSK splicing is altered in that level of MuSK transcripts including exons 3 and 4 remains substantially unchanged and level of MuSK transcripts including exons 6 and 7 is decreased.

17. (canceled)

18. The composition of claim 1, wherein MuSK splicing is altered in that total level of MuSK transcripts remained substantially unchanged and level of MuSK transcripts including exons 6 and 7 is decreased.

19-23. (canceled)

24. The composition of claim 1, wherein the level of MuSK transcripts including exons 6 and 7 or the level of MuSK protein forms including sequences encoded by exons 6 and 7, or both, decreases by greater than 60%, greater than 70%, greater than 80%, or greater than 90% and the level of MuSK transcripts including exons 3 and 4 or the level of MuSK protein forms including sequences encoded by exons 3 and 4, or both, decreases by less than 40%, less than 30%, less than 20%, or less than 10%.

25-31. (canceled)

32. The composition of claim 1, wherein the oligonucleotides target a region on the MuSK genomic sequence corresponding to positions 83776-83800 and/or 83854-83878 of SEQ ID NO: 77.

33. The composition of claim 1, wherein the oligonucleotides target a region on the MuSK genomic sequence within or comprising at least a portion of sequence ACTCTGTCAGGTTTCTTCTGGGTCCATTCAAGAGAGTGTGAAAGACCGAGTGATTGA CTCAAGAC (region 1, SEQ ID: 126).

34-36. (canceled)

37. The composition of claim 1, wherein the oligonucleotides target a portion of MuSK transcript comprising a sequence that is identical to 15, 16, 17, 18, 19, 20, 21, 22, 23, 24, 25, 26, 27, 28, 29, or 30 consecutive bases of region 1, SEQ ID: 126.

38. The composition of claim 1, wherein the oligonucleotides target a portion of MuSK transcript comprising a sequence that is identical to 19 21-25 consecutive bases of region 1, SEQ ID: 126.

39-49. (canceled)

50. The composition of claim 1, wherein the oligonucleotides target a region on the MUSK genomic sequence within or comprising at least a portion of sequence GGGGAGAAGTTCAGTACTGCCAAGGCTGCAGCCACCATCAGCATAGCAGGTAGGAT GCCCCTTCACATTTG (region 2, SEQ ID 211).

51-53. (canceled)

54. The composition of claim 1, wherein the oligonucleotides target a portion of MuSK transcript comprising a sequence that is identical to 15, 16, 17, 18, 19, 20, 21, 22, 23, 24, 25, 26, 27, 28, 29, or 30 consecutive bases of region 2, SEQ ID: 211.

55-97. (canceled)

98. The composition of claim 1, wherein the base sequence of the oligonucleotide comprises from 5′ to 3′:

| SEQ | Oligo | |
|---|---|---|
| ID | ID | 5′ to 3′ Sequence |
| 1 | Bld1 | GCTAGGGTGGTCTTTTAGAAATGCA |
| 2 | Bld2 | GGTCAAGCTAGGGTGGTCTTTTAGA |
| 3 | Bld3 | CTGCAGGAAATGGTCAAGCTAGGGT |
| 4 | Bld4 | GAAGTGGTGAGTGACGCTCCTGCAG |
| 5 | Bld5 | GTTAGGAAGACAGAAGTGGTGAGTG |
| 6 | Bld6 | ATCCTGGCAAAAACTGTTAGGAAGA |
| 7 | Bld7 | GTGGGATTCAGGAGCCCGCAGGATC |
| 8 | Bld8 | GGTGACATTGTGGGATTCAGGAGCC |
| 9 | Bld9 | GGAGCCAAAGGTGACATTGTGGGAT |
| 10 | Bld10 | GGTCACAAAGGAGCCAAAGGTGACA |
| 11 | Bld11 | ACAGTGCAGGGTCACAAAGGAGCCA |
| 12 | Bld12 | CTGTTGCTGTACAGTGCAGGGTCAC |
| 13 | Bld13 | GGGACAGGAATGCCTGTTGCTGTAC |
| 14 | Bld14 | CAGGTGATGGTGGGGACAGGAATGC |
| 15 | Bld15 | CCGTTTTCAATCCAGGTGATGGTGG |
| 16 | Bld16 | TGACACTCACAGCATTTCCGTTTTC |
| 17 | Bld17 | AAGTCCCCACACACATGACACTCAC |
| 18 | Bld18 | GGTCTTCCCCAGACAAGTCCCCACA |
| 19 | Bld19 | ACTATGTCAGTAGATTTGAAGGGAA |
| 20 | Bld20 | TCCCACTATACTATGTCAGTAGATT |
| 21 | Bld21 | TCAGTCAAGGATTTCCCACTATACT |
| 22 | Bld22 | AAAAGAACTCAGTCAAGGATTTCCC |
| 23 | Bld23 | GTAAAGGAAAATAAAAGAACTCAGT |
| 24 | Bld24 | AACCTGACAGAGTAAAGGAAAATAA |
| 25 | B1d25 | GGACCCAGAAGAAACCTGACAGAGT |
| 26 | Bld26 | CACTCTCTTGAATGGACCCAGAAGA |
| 27 | Bld27 | CACTCGGTCTTTCACACTCTCTTGA |
| 28 | Bld28 | GTCTTGAGTCAATCACTCGGTCTTT |
| 29 | Bld29 | GATAAACAGCTGCAGTCTTGAGTCA |
| 30 | Bld30 | AGTCCTGGCTTGGTGATAAACAGCT |
| 31 | Bld31 | ATGTGTAGAGTCCTGGCTTGGTGAT |
| 32 | Bld32 | GTAGCTATGCATGTGTAGAGTCCTG |
| 33 | Bld33 | TGCTTATTGGTAGCTATGCATGTGT |
| 34 | Bld34 | ACTTCTCCCCATGCTTATTGGTAGC |
| 35 | Bld35 | CCTTGGCAGTACTGAACTTCTCCCC |
| 36 | Bld36 | CCTGCTATGCTGATGGTGGCTGCAG |
| 37 | Bld37 | GGGCATCCTACCTGCTATGCTGATG |
| 38 | Bld38 | GCAAATGTGAAGGGGCATCCTACCT |
| 127 | Bld51 | TTGAATGGACCCAGAAGAAA |
| 128 | Bld52 | CTTGAATGGACCCAGAAGAA |
| 129 | Bld53 | TCTTGAATGGACCCAGAAGA |
| 130 | Bld54 | CTCTTGAATGGACCCAGAAG |
| 131 | Bld55 | TCTCTTGAATGGACCCAGAA |
| 132 | Bld56 | CTCTCTTGAATGGACCCAGA |
| 133 | Bld57 | ACTCTCTTGAATGGACCCAG |
| 134 | Bld58 | CACTCTCTTGAATGGACCCA |
| 135 | Bld59 | ACACTCTCTTGAATGGACCC |
| 136 | Bld60 | CACACTCTCTTGAATGGACC |
| 137 | Bld61 | TCACACTCTCTTGAATGGAC |
| 138 | Bld62 | TTCACACTCTCTTGAATGGA |
| 139 | Bld63 | TTTCACACTCTCTTGAATGG |
| 140 | Bld64 | CTTTCACACTCTCTTGAATG |
| 141 | Bld65 | TCTTTCACACTCTCTTGAAT |
| 142 | Bld66 | GTCTTTCACACTCTCTTGAA |
| 159 | Bld25-1 | CCAGAAGAAACCTGACAGAGTAAAG |
| 160 | Bld25-2 | ACCCAGAAGAAACCTGACAGAGTAA |
| 161 | Bld25-3 | ATGGACCCAGAAGAAACCTGACAGA |
| 162 | Bld25-4 | GAATGGACCCAGAAGAAACCTGACA |
| 163 | Bld25-5 | CTTGAATGGACCCAGAAGAAACCTG |
| 164 | Bld26-1 | CTCTTGAATGGACCCAGAAGAAACC |
| 165 | Bld26-2 | CTCTCTTGAATGGACCCAGAAGAAA |
| 166 | Bld26-3 | CACACTCTCTTGAATGGACCCAGAA |
| 167 | Bld26-4 | TTCACACTCTCTTGAATGGACCCAG |
| 177 | Bld25-A | ACCCAGAAGAAACCTGACAGAGT |
| 178 | Bld25-B | CCAGAAGAAACCTGACAGAGT |
| 179 | Bld25-C | GGACCCAGAAGAAACCTGACAGA |
| 180 | Bld25-D | ACCCAGAAGAAACCTGACAGA |
| 181 | Bld25-E | GGACCCAGAAGAAACCTGACA |
| 182 | Bld25-5-A | GAATGGACCCAGAAGAAACCTGA |
| 183 | Bld25-5-B | ATGGACCCAGAAGAAACCTGA |
| 184 | Bld25-5-C | TTGAATGGACCCAGAAGAAACCT |
| 185 | Bld25-5-D | GAATGGACCCAGAAGAAACCT |
| 186 | Bld25-5-E | TTGAATGGACCCAGAAGAAAC |
| 187 | Bld26-2-A | CTCTTGAATGGACCCAGAAGAAA |
| 188 | Bld26-2-B | CTTGAATGGACCCAGAAGAAA |
| 189 | Bld26-2-C | CTCTCTTGAATGGACCCAGAAGA |
| 190 | Bld26-2-D | CTCTCTTGAATGGACCCAGAA |
| 191 | Bld26-B | CTCTTGAATGGACCCAGAAGA |
| 192 | Bld26-C | CACTCTCTTGAATGGACCCAGAA, or |
| 193 | Bld26-D | CACTCTCTTGAATGGACCCAG. |

99. The composition of claim 1, wherein the oligonucleotide is complementary to a nucleotide sequence that is at least 90% identical to any one of SEQ ID NOs: 39-76 and 212-253.

100. The composition of claim 98, wherein the oligonucleotides comprise one or more types of base modifications, sugar modification, and internucleotidic linkage modifications.

101-102. (canceled)

103. The composition of claim 100, wherein the internucleotidic linkages of the oligonucleotide comprise natural phosphate, phosphorothioate, or phosphodithioate linkages.

104. The composition of claim 103, wherein each internucleotidic linkage of the oligonucleotide is a phosphorothioate linkage.

105-108. (canceled)

109. The composition of claim 100, wherein the oligonucleotides comprise one or more sugar modifications.

110. The composition of claim 105, wherein the sugar modification is moiety has a 2′-modification.

111-112. (canceled)

113. The composition of claim 110, wherein the 2′-modification is 2′-MOE.

114-118. (canceled)

119. The composition of claim 113, wherein each sugar of the oligonucleotide is a 2′-MOE modified sugar.

120-124. (canceled)

125. The composition of claim 1, wherein the oligonucleotide has the structure from 5′ to 3′ of:

| Oligo | SEQ ID | |
|---|---|---|
| ID | NO: | 5′ to 3′ Sequence |
| Bld1 | 78 | G*C*T*A*G*G*G*T*G*G*T*C*T*T*T*T*A*G*A*A*A*T*G*C*A |
| Bld2 | 79 | G*G*T*C*A*A*G*C*T*A*G*G*G*T*G*G*T*C*T*T*T*T*A*G*A |
| Bld3 | 80 | C*T*G*C*A*G*G*A*A*A*T*G*G*T*C*A*A*G*C*T*A*G*G*G*T |
| Bld4 | 81 | G*A*A*G*T*G*G*T*G*A*G*T*G*A*C*G*C*T*C*C*T*G*C*A*G |
| Bld5 | 82 | G*T*T*A*G*G*A*A*G*A*C*A*G*A*A*G*T*G*G*T*G*A*G*T*G |
| Bld6 | 83 | A*T*C*C*T*G*G*C*A*A*A*A*A*C*T*G*T*T*A*G*G*A*A*G*A |
| Bld7 | 84 | G*T*G*G*G*A*T*T*C*A*G*G*A*G*C*C*C*G*C*A*G*G*A*T*C |
| Bld8 | 85 | G*G*T*G*A*C*A*T*T*G*T*G*G*G*A*T*T*C*A*G*G*A*G*C*C |
| Bld9 | 86 | G*G*A*G*C*C*A*A*A*G*G*T*G*A*C*A*T*T*G*T*G*G*G*A*T |
| Bld10 | 87 | G*G*T*C*A*C*A*A*A*G*G*A*G*C*C*A*A*A*G*G*T*G*A*C*A |
| Bld11 | 88 | A*C*A*G*T*G*C*A*G*G*G*T*C*A*C*A*A*A*G*G*A*G*C*C*A |
| Bld12 | 89 | C*T*G*T*T*G*C*T*G*T*A*C*A*G*T*G*C*A*G*G*G*T*C*A*C |
| Bld13 | 90 | G*G*G*A*C*A*G*G*A*A*T*G*C*C*T*G*T*T*G*C*T*G*T*A*C |
| Bld14 | 91 | C*A*G*G*T*G*A*T*G*G*T*G*G*G*G*A*C*A*G*G*A*A*T*G*C |
| Bld15 | 92 | C*C*G*T*T*T*T*C*A*A*T*C*C*A*G*G*T*G*A*T*G*G*T*G*G |
| Bld16 | 93 | T*G*A*C*A*C*T*C*A*C*A*G*C*A*T*T*T*C*C*G*T*T*T*T*C |
| Bld17 | 94 | A*A*G*T*C*C*C*C*A*C*A*C*A*C*A*T*G*A*C*A*C*T*C*A*C |
| Bld18 | 95 | G*G*T*C*T*T*C*C*C*C*A*G*A*C*A*A*G*T*C*C*C*C*A*C*A |
| Bld19 | 96 | A*C*T*A*T*G*T*C*A*G*T*A*G*A*T*T*T*G*A*A*G*G*G*A*A |
| Bld20 | 97 | T*C*C*C*A*C*T*A*T*A*C*T*A*T*G*T*C*A*G*T*A*G*A*T*T |
| Bld21 | 98 | T*C*A*G*T*C*A*A*G*G*A*T*T*T*C*C*C*A*C*T*A*T*A*C*T |
| Bld22 | 99 | A*A*A*A*G*A*A*C*T*C*A*G*T*C*A*A*G*G*A*T*T*T*C*C*C |
| Bld23 | 100 | G*T*A*A*A*G*G*A*A*A*A*T*A*A*A*A*G*A*A*C*T*C*A*G*T |
| Bld24 | 101 | A*A*C*C*T*G*A*C*A*G*A*G*T*A*A*A*G*G*A*A*A*A*T*A*A |
| Bld25 | 102 | G*G*A*C*C*C*A*G*A*A*G*A*A*A*C*C*T*G*A*C*A*G*A*G*T |
| Bld26 | 103 | C*A*C*T*C*T*C*T*T*G*A*A*T*G*G*A*C*C*C*A*G*A*A*G*A |
| Bld27 | 104 | C*A*C*T*C*G*G*T*C*T*T*T*C*A*C*A*C*T*C*T*C*T*T*G*A |
| Bld28 | 105 | G*T*C*T*T*G*A*G*T*C*A*A*T*C*A*C*T*C*G*G*T*C*T*T*T |
| Bld29 | 106 | G*A*T*A*A*A*C*A*G*C*T*G*C*A*G*T*C*T*T*G*A*G*T*C*A |
| Bld30 | 107 | A*G*T*C*C*T*G*G*C*T*T*G*G*T*G*A*T*A*A*A*C*A*G*C*T |
| Bld31 | 108 | A*T*G*T*G*T*A*G*A*G*T*C*C*T*G*G*C*T*T*G*G*T*G*A*T |
| Bld32 | 109 | G*T*A*G*C*T*A*T*G*C*A*T*G*T*G*T*A*G*A*G*T*C*C*T*G |
| Bld33 | 110 | T*G*C*T*T*A*T*T*G*G*T*A*G*C*T*A*T*G*C*A*T*G*T*G*T |
| Bld34 | 111 | A*C*T*T*C*T*C*C*C*C*A*T*G*C*T*T*A*T*T*G*G*T*A*G*C |
| Bld35 | 112 | C*C*T*T*G*G*C*A*G*T*A*C*T*G*A*A*C*T*T*C*T*C*C*C*C |
| Bld36 | 113 | C*C*T*G*C*T*A*T*G*C*T*G*A*T*G*G*T*G*G*C*T*G*C*A*G |
| Bld37 | 114 | G*G*G*C*A*T*C*C*T*A*C*C*T*G*C*T*A*T*G*C*T*G*A*T*G |
| Bld38 | 115 | G*C*A*A*A*T*G*T*G*A*A*G*G*G*G*C*A*T*C*C*T*A*C*C*T, |
| Bld51 | 143 | T*T*G*A*A*T*G*G*A*C*C*C*A*G*A*A*G*A*A*A |
| Bld52 | 144 | C*T*T*G*A*A*T*G*G*A*C*C*C*A*G*A*A*G*A*A |
| Bld53 | 145 | T*C*T*T*G*A*A*T*G*G*A*C*C*C*A*G*A*A*G*A |
| Bld54 | 146 | C*T*C*T*T*G*A*A*T*G*G*A*C*C*C*A*G*A*A*G |
| Bld55 | 147 | T*C*T*C*T*T*G*A*A*T*G*G*A*C*C*C*A*G*A*A |
| Bld56 | 148 | C*T*C*T*C*T*T*G*A*A*T*G*G*A*C*C*C*A*G*A |
| Bld57 | 149 | A*C*T*C*T*C*T*T*G*A*A*T*G*G*A*C*C*C*A*G |
| Bld58 | 150 | C*A*C*T*C*T*C*T*T*G*A*A*T*G*G*A*C*C*C*A |
| Bld59 | 151 | A*C*A*C*T*C*T*C*T*T*G*A*A*T*G*G*A*C*C*C |
| Bld60 | 152 | C*A*C*A*C*T*C*T*C*T*T*G*A*A*T*G*G*A*C*C |
| Bld61 | 153 | T*C*A*C*A*C*T*C*T*C*T*T*G*A*A*T*G*G*A*C |
| Bld62 | 154 | T*T*C*A*C*A*C*T*C*T*C*T*T*G*A*A*T*G*G*A |
| Bld63 | 155 | T*T*T*C*A*C*A*C*T*C*T*C*T*T*G*A*A*T*G*G |
| Bld64 | 156 | C*T*T*T*C*A*C*A*C*T*C*T*C*T*T*G*A*A*T*G |
| Bld65 | 157 | T*C*T*T*T*C*A*C*A*C*T*C*T*C*T*T*G*A*A*T |
| Bld66 | 158 | G*T*C*T*T*T*C*A*C*A*C*T*C*T*C*T*T*G*A*A |
| Bld25-1 | 168 | C*C*A*G*A*A*G*A*A*A*C*C*T*G*A*C*A*G*A*G*T*A*A*A*G* |
| Bld25-2 | 169 | A*C*C*C*A*G*A*A*G*A*A*A*C*C*T*G*A*C*A*G*A*G*T*A*A* |
| Bld25-3 | 170 | A*T*G*G*A*C*C*C*A*G*A*A*G*A*A*A*C*C*T*G*A*C*A*G*A* |
| Bld25-4 | 171 | G*A*A*T*G*G*A*C*C*C*A*G*A*A*G*A*A*A*C*C*T*G*A*C*A* |
| Bld25-5 | 172 | C*T*T*G*A*A*T*G*G*A*C*C*C*A*G*A*A*G*A*A*A*C*C*T*G* |
| Bld26-1 | 173 | C*T*C*T*T*G*A*A*T*G*G*A*C*C*C*A*G*A*A*G*A*A*A*C*C* |
| Bld26-2 | 174 | C*T*C*T*C*T*T*G*A*A*T*G*G*A*C*C*C*A*G*A*A*G*A*A*A* |
| Bld26-3 | 175 | C*A*C*A*C*T*C*T*C*T*T*G*A*A*T*G*G*A*C*C*C*A*G*A*A* |
| Bld26-4 | 176 | T*T*C*A*C*A*C*T*C*T*C*T*T*G*A*A*T*G*G*A*C*C*C*A*G* |
| Bld25-A | 194 | A*C*C*C*A*G*A*A*G*A*A*A*C*C*T*G*A*C*A*G*A*G*T |
| Bld25-B | 195 | C*C*A*G*A*A*G*A*A*A*C*C*T*G*A*C*A*G*A*G*T |
| Bld25-C | 196 | G*G*A*C*C*C*A*G*A*A*G*A*A*A*C*C*T*G*A*C*A*G*A |
| Bld25-D | 197 | A*C*C*C*A*G*A*A*G*A*A*A*C*C*T*G*A*C*A*G*A |
| Bld25-E | 198 | G*G*A*C*C*C*A*G*A*A*G*A*A*A*C*C*T*G*A*C*A |
| Bld25-5-A | 199 | G*A*A*T*G*G*A*C*C*C*A*G*A*A*G*A*A*A*C*C*T*G*A |
| Bld25-5-B | 200 | A*T*G*G*A*C*C*C*A*G*A*A*G*A*A*A*C*C*T*G*A |
| Bld25-5-C | 201 | T*T*G*A*A*T*G*G*A*C*C*C*A*G*A*A*G*A*A*A*C*C*T |
| Bld25-5-D | 202 | G*A*A*T*G*G*A*C*C*C*A*G*A*A*G*A*A*A*C*C*T |
| Bld25-5-E | 203 | T*T*G*A*A*T*G*G*A*C*C*C*A*G*A*A*G*A*A*A*C |
| Bld26-2-A | 204 | C*T*C*T*T*G*A*A*T*G*G*A*C*C*C*A*G*A*A*G*A*A*A |
| Bld26-2-B | 205 | C*T*T*G*A*A*T*G*G*A*C*C*C*A*G*A*A*G*A*A*A |
| Bld26-2-C | 206 | C*T*C*T*C*T*T*G*A*A*T*G*G*A*C*C*C*A*G*A*A*G*A |
| Bld26-2-D | 207 | C*T*C*T*C*T*T*G*A*A*T*G*G*A*C*C*C*A*G*A*A |
| Bld26-B | 208 | C*T*C*T*T*G*A*A*T*G*G*A*C*C*C*A*G*A*A*G*A |
| Bld26-C | 209 | C*A*C*T*C*T*C*T*T*G*A*A*T*G*G*A*C*C*C*A*G*A*A, or |
| Bld26-D | 210 | C*A*C*T*C*T*C*T*T*G*A*A*T*G*G*A*C*C*C*A*G. |

wherein * represents a phosphorothioate linkage, and each sugar of the oligonucleotides is a 2′-MOE modified sugar.

126. An oligonucleotide composition comprising a mixture of two or more oligonucleotides according to claim 125.

127. (canceled)

128. A pharmaceutical composition comprising a therapeutically effective amount of an oligonucleotide, and at least one pharmaceutically acceptable inactive ingredient selected from pharmaceutically acceptable diluents, pharmaceutically acceptable excipients, and pharmaceutically acceptable carriers, wherein the oligonucleotide comprises an oligonucleotide of claim 1.

129-142. (canceled)

143. A method of treating a subject suffering from one or more features of neurodegenerative diseases, the method comprising a step of administering to a subject a pharmaceutical composition that comprises or delivers a composition of claim 1.

144. (canceled)

145. A method of treating a subject suffering from one or more features of neuromuscular dysfunction or a muscular dystrophy, the method comprising a step of administering to a subject a pharmaceutical composition that comprises or delivers a composition of claim 1.

146. (canceled)

147. A method of treating muscle fibrosis, the method comprising a step of administering to a subject a pharmaceutical composition that comprises or delivers a composition of claim 1.

148-167. (canceled)
